(19) United States
(12) Patent Application Publication
(10) Pub. No.: US 2022/0290121 A1
(43) Pub. Date: Sep. 15, 2022

(54) Combinatorial Adenine and Cytosine DNA Base Editors

(71) Applicant: The General Hospital Corporation, Boston, MA (US)

(72) Inventors: J. Keith Joung, Winchester, MA (US); Ronghao Zhou, Charlestown, MA (US); Julian Grunewald, Charlestown, MA (US)

(21) Appl. No.: 17/638,133

(22) PCT Filed: Aug. 31, 2020
(86) PCT No.: PCT/US2020/048825

§ 371 Date: Feb. 24, 2022

Related U.S. Application Data
(60) Provisional application No. 63/023,192, filed on May 11, 2020.
(60) Provisional application No. 62/894,612, filed on Aug. 30, 2019.

(51) Int. Cl. C12N 15/10; C12P 21/02; C12P 19/34; C12N 9/78; C12N 9/22

(52) U.S. Cl. CPC C12N 15/102; C12P 21/02; C12P 19/34; C12N 9/78; C12N 9/22; C12Y 305/04004; C12Y 305/04005; C07K 2319/09; C07K 2319/80; C12N 2310/20

(57) ABSTRACT

Engineered bifunctional adenine and cytosine base editor (BACE) variants that enable expanded amino acid modifications and methods of using the same. Described herein are fusion proteins containing adenosine deaminases, cytidine deaminases, catalytically impaired CRISPR-Cas proteins (e.g., Cas9, CasX or Cas12 nucleases), linkers, nuclear localization signals (NLSs) and uracil-n-glycosylase inhibitors (UGIs) that enable the CRISPR-guided programmable introduction of simultaneous A-to-G (T-to-C) and C-to-T (G-to-A) substitutions in DNA.

### CLAIM OF PRIORITY

This application claims the benefit of U.S. Provisional Patent Application Ser. Nos. 62/894,612, filed on 30 Aug. 2019, and 63/023,192, filed on 11 May 2020. The entire contents of the foregoing are hereby incorporated by reference.

### FEDERALLY SPONSORED RESEARCH OR DEVELOPMENT

This invention was made with Government support under Grant No. HG009490 awarded by the National Institutes of Health and contract HR0011-17-2-0042 awarded by the Defense Advanced Research Projects Agency of the Department of Defense. The Government has certain rights in the invention.

### TECHNICAL FIELD

Described herein are fusion proteins containing adenosine deaminases, cytidine deaminases, catalytically impaired CRISPR-Cas proteins (e.g., Cas9, CasX or Cas12 nucleases), linkers, nuclear localization signals (NLSs) and uracil-n-glycosylase inhibitors (UGIs) that enable the CRISPR-guided programmable introduction of simultaneous A-to-G (T-to-C) and C-to-T (G-to-A) substitutions in DNA.

### BACKGROUND

DNA base editors represent a new class of genome editing tools that enable the programmable installation of single or multiple base substitutions. Current cytosine (CBE) and adenine (ABE) generations of base editors allow for the targeted deamination of cytosines and adenines that get exposed on ssDNA by RNA-guided CRISPR-Cas proteins¹⁻⁴. The majority of disease-associated genetic perturbations known to date are point mutations, also known as single nucleotide variants (SNVs). Current iterations of CBEs and ABEs can target disease-relevant transition mutations and revert them to the original genotype, e.g., correcting G-to-A (C-to-T) mutations using ABE. However, if the experimental or therapeutic goal of base editing is to modify amino acid (AA) residues, both CBEs and ABEs are limited.

### SUMMARY

Fusion proteins that contain both adenine and cytidine deaminases expand the potential for AA modification by enabling the programmable alteration of one to three neighboring codons by installing both A-to-G and C-to-T mutations side-by-side. These bifunctional adenine and cytosine editors (BACE) also allow for the correction of double or triple nucleotide variants (DNVs or TNVs) that are associated with disease.

Described herein are CRISPR-guided bifunctional adenine and cytosine base editors (BACEs) that enable the simultaneous installation of adenine-to-guanine and cytosine-to-thymine base edits within the same editing window at the ssDNA bubble generated by RNA-guided fusion proteins that contain both adenine (e.g., E. coli TadA) and cytosine (e.g., pmCDA1 or rAPOBEC1) deaminases as well as CRISPR-Cas proteins (e.g., S. pyogenes Cas9). The exemplary SpCas9-based synchronous programmable adenine and cytosine base editor (SPACE) fusion protein system described herein comprises a programmable DNA-binding domain fused to an adenosine deaminase, e.g., E. coli TadA or previously described engineered TadA variants to decrease RNA editing activity while still preserving DNA editing activity (SECURE or RRE variants) as well as to a cytidine deaminase, e.g., pmCDA1 or rAPOBEC1 or human APOBEC3A or human AID or human APOBEC3G or previously described engineered variants of these deaminases (e.g., rAPOBEC1 mutations from SECURE-BE3) with reduced RNA editing activity and preserved DNA editing capabilities⁵⁻⁹.

Thus, provided herein are bifunctional adenine and cytosine base editor (BACEs) comprising:

(i) an adenosine deaminase domain, e.g., a wild type (SEQ ID NO: 98) and/or engineered adenosine deaminase TadA monomer or dimer (e.g., homodimeris or heterodimeric TadA domains from ABEmax (SEQ ID NO:226), ABE7.10 (SEQ ID NO:227), or ABE8e (SEQ ID NO: 145); other options include monomer or dimer TadAs from ABEs 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10 (SEQ ID NO: 139), or ABEmax (SEQ ID NO: 228), or ABE8.8 (SEQ ID NO: 148), ABE8.13 (SEQ ID NO: 149), ABE8.17 (SEQ ID NO: 150), ABE8.20 (SEQ ID NO: 151), ABE8e (SEQ ID NO: 145)—as well as K20A/R21A, V82G, or V106W variants thereof), E. coli TadA monomer, or homo- or heterodimers thereof fused to the N or C terminus, bearing one or more mutations in either or both monomers (e.g., the TadA mutant used in miniABEmax-V82G (SEQ ID 223), miniABEmax-K20A/R21A (SEQ ID 224), miniABEmax-V106W (SEQ ID 225) or any other variant from Tables C, N, and O), that decrease RNA editing activity while preserving DNA editing activity;

(ii) a cytidine deaminase from Tables A and B (e.g., pmCDA1, rat APOBEC1, human APOBEC3A, or human AID) or variations thereof with reduced RNA off-target editing, one or multiple uracil-n-clycosalyse inhibitors (UGIs); and

(iii) a programmable DNA binding domain (e.g., Cas9-D10A); and (iv) optionally further comprising one or more nuclear localization sequences (e.g., NLSs such as a bipartite NLS comprising the sequence KRTADGSEFEPKKKRKV (SEQ ID NO:229); an SV40 large T antigen NLS (PKKKRRV (SEQ ID NO:221)); or a nucleoplasmin NLS (KRPAATKKAGQAKKKK (SEQ ID NO:222)).

Exemplary BACEs include those provided in SEQ ID NOs:140-144.

In some embodiments, the adenosine deaminase comprises one or more mutations corresponding to E. coli TadA mutations in one or more TadA monomers shown in Table N, or an homologue or orthologue thereof (e.g., a TadA protein in Table C).

In some embodiments, the cytidine deaminase rat APOBEC1 (or any one of its ortho- or paralogues listed in Tables A or B) bears one or more mutations that decrease RNA editing activity while preserving DNA editing activity, wherein the mutations are at amino acid positions that correspond to residues P29, R33, K34, E181, and/or L182 of rat apolipoprotein B mRNA editing enzyme, catalytic polypeptide-like 1 (rAPOBEC1, SEQ ID NO:67).

In some embodiments, the cytidine deaminase rat APOBEC1 (e.g., or any one of its ortho- or paralogues listed in Tables A or B) bears one or more mutations at positions: P29F, P29T, R33A, K34A, R33A+K34A (double mutant), E181Q and/or L182A of SEQ ID NO:67 (rAPOBEC1, Rattus norvegicus APOBEC1).

In some embodiments, the BACE further include one or more mutations at its cytidine deaminase rat APOBEC1 (or any one of its ortho- or paralogues listed in Tables A or B) residues corresponding to E24, V25; R118, Y120, H121, R126; W224-K229; P168-1186; L173+L180; R15, R16, R17, to K15-17 & A15-17; Deletion E181-L210; P190+P191; Deletion L210-K229 (C-terminal); and/or Deletion S2-L14 (N-terminal) of SEQ ID NO:67, Table O.

In some embodiments, the BACE includes a linker between the adenosine deaminase monomer and/or between the adenosine deaminase monomer or single-chain dimers and the programmable DNA binding domain.

In some embodiments, the BACE includes a linker between the programmable DNA binding domain and the cytidine deaminase monomer (e.g., pmCDA1 or rAPOBEC1 or hA3A or hAID) or dimer.

In some embodiments, the BACE includes an N-terminal adenosine deaminase fusion (e.g., mutant TadA* monomer or dimer) and a C-terminal cytidine deaminase fusion (e.g., pmCDA1 or rAPOBEC1 or hA3A or AID).

In some embodiments, the BACE includes an N-terminal cytidine deaminase fusion (e.g., pmCDA1 or rAPOBEC1 or hA3A or AID) and a C-terminal adenosine deaminase fusion (e.g., mutant TadA* monomer or dimer).

In some embodiments, the BACE includes a heterodimeric combined N-terminal adenosine and cytidine deaminase fusion (e.g., pmCDA1 or rAPOBEC1 or hA3A or AID fused to TadA monomers or dimers with a linker) or a heterodimeric combined C-terminal adenosine and cytidine deaminase fusion (e.g., pmCDA1 or rAPOBEC1 or hA3A or AID fused to TadA monomers or dimers with a linker). In both N- and C-terminal positions of these “hybrid fusion deaminase designs” the deaminases can be fused in either of these two orders: NH2-cytidine deaminase-linker-adenosine deaminase or NH2-adenosine deaminase-linker-cytidine deaminase.

In some embodiments, the programmable DNA binding domain is selected from the group consisting of engineered C2H2 zinc-fingers, transcription activator effector-like effectors (TALEs), and Clustered Regularly Interspaced Short Palindromic Repeats (CRISPR) Cas RNA-guided nucleases (RGNs) and variants thereof (e.g., Tables L and M) (e.g., a “SPACE” as described herein).

In some embodiments, the CRISPR RGN is an ssDNA nickase or is catalytically inactive, e.g., a Cas9, CasX or Cas12a that has ssDNA nickase activity or is catalytically inactive.

Further provided herein are base editing systems comprising: (i) a BACE as described herein, wherein the programmable DNA binding domain is a CRISPR Cas RGN or a variant thereof; and (ii) at least one guide RNA compatible with the base editor that directs the base editor to a target sequence.

Also described herein are isolated nucleic acids encoding any of the BACEs or base editing systems described herein, as well as vectors comprising an isolated nucleic acid described herein.

Also provided are isolated host cells, preferably mammalian (e.g., human) host cells, comprising any of the nucleic acids described herein. In some embodiments, the isolated host cells described herein express a BACE as described herein.

Further, provided herein are methods for deaminating a selected adenine and/or cytosine in a nucleic acid; the methods include contacting the nucleic acid with a BACE, a base editing system, an isolated nucleic acid, a vector, or an isolated host cell described herein.

Additionally provided herein are compositions comprising a purified BACE, a base editing system, an isolated nucleic acid, a vector, and/or an isolated host cell described herein. In some embodiments, the composition includes one or more ribonucleoprotein (RNP) complexes, e.g., comprising a BACE and compatible gRNA.

Also provided herein are methods of inducing an amino acid change in a polypeptide. The methods include contacting a nucleotide that encodes the polypeptide the amino acid sequence of which is to be changed with a BACE, a base editing system, an isolated nucleic acid, a vector, or an isolated host cell described herein, preferably wherein the amino acid change comprises one of the amino acid changes listed in Table D, and optionally wherein the amino acid change is one that can or cannot be targeted by CBE and/or ABE.

In some embodiments, the BACE is used to correct or model (create) specific disease-related mutations as shown in Table E-K.

In some embodiments, the BACE or SPACE comprises one or more uracil-N-glycosylase inhibitors (UGIs). In some embodiments, the BACE or SPACE comprise a linker between the adenosine deaminase and the programmable DNA binding domain as well as the cytidine deaminase and the DNA binding domain. In some embodiments, the TadA domain can be monomeric, homodimeric or heterodimeric and contain all combinations of wild type (WT) E. coli TadA, or mutant variants of TadA.

In some embodiments the two deaminase domains can be located at the C-terminus (e.g., pmCDA1) and N-terminus (TadA) or vice versa or they can both be located at the C- or N-terminus.

In some embodiments, the programmable DNA binding domain is selected from the group consisting of engineered C2H2 zinc-fingers, transcription activator effector-like effectors (TALEs), and Clustered Regularly Interspaced Short Palindromic Repeats (CRISPR)-Cas RNA-guided nucleases (RGNs) and variants thereof.

In some embodiments, the CRISPR-Cas RGN is an ssDNA nickase or is catalytically inactive, e.g., a Cas9 or Cas12a that is catalytically inactive or has ssDNA nickase activity.

Also provided herein are base editing systems comprising (i) the adenine base editors described herein, wherein the programmable DNA binding domain is a CRISPR Cas RGN or a variant thereof; and (ii) at least one guide RNA compatible with the base editor that directs the base editor to a target sequence.

Also provided are isolated nucleic acids encoding the adenine base editors; vectors comprising the isolated nucleic acids; and isolated host cells, preferably mammalian host cells, comprising the nucleic acids. In some embodiments, the isolated host cell expresses an adenine base editor.

Further, provided herein are methods for deaminating a selected adenine in a nucleic acid, the method comprising contacting the nucleic acid with an adenine base editor or base editing system as described herein.

Also provided are compositions comprising a purified adenine base editor or base editing system as described herein. In some embodiments, the composition comprises one or more ribonucleoprotein (RNP) complexes, e.g., comprising a SPACE and compatible guide RNA.

These methods can be used for generating two or more sets of nucleic acids, each set comprising a plurality of sequences, wherein each set comprises one or more nucleic acids having the same sequence, and wherein each set differs from each of the other sets by at least one nucleotide. These methods include (i) providing a first nucleic acid comprising a first sequence, e.g., a reference or wild type sequence; (ii) contacting the first nucleic acids with a BACE as described herein, wherein the programmable DNA binding domain is a CRISPR Cas RGN or a variant thereof; and a least one guide RNA compatible with the base editor that directs the base editor to modify a selected nucleotide in the first sequence; and (iii) isolating a second nucleic acid comprising a sequence comprising the selected modification in the nucleotide sequence, to provide a second set of nucleic acids. The methods can include amplifying the second nucleic acids. Steps (i)-(iii) can optionally be repeated until a desired number of sets is obtained, e.g., until enough sets are obtained to include at least one set with a mutation at each position in a selection region of the sequence of the nucleic acid.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Schematic illustrating the architecture of SPACE. Schematic illustrating a SECURE miniABEmax-V82G or -K20A/R21A derived TadA* monomer fused to a nickase Cas9 (nCas9)-gRNA complex that directs A to I deamination in the ssDNA generated by nCas9-induced R-loop formation. On the C-terminus, pmCDA1 (or other cytidine deaminase with low RNA off-target editing, e.g., SECURE-BE3 rAPOBEC1 R33A or R33A/K34A and one or more UGI domains were fused to the DNA targeting protein.

FIG. 2. SPACE induces concurrent A-to-G and C-to-T base edits in human HEK293T cells at 28 sites. Heat maps showing on-target DNA A-to-G and C-to-T editing frequencies with 28 gRNAs (n=4 independent replicates). Numbering at the bottom represents the position of the respective base in the protospacer sequence with 1 being the most PAM-distal.

FIGS. 3A-C. A-to-G or C-to-T editing distributions of SPACE, miniABEmax-V82G, and Target-AID. Box and dot plots indicating the aggregate distribution of A-to-G and C-to-T edits across the entire protospacer with SPACE (A), miniABEmax-V82G (B), and Target-AID (C) using 28 gRNAs. In box plots, the box spans the interquartile range (IQR) (first to third quartiles), the horizontal line shows the median (second quartile) and whiskers extend to ±1.5×IQR. Single dots represent individual replicates. Graph shows the same data as shown in FIG. 2 (n=4).

FIG. 4. Allele frequency tables of DNA on-target editing by SPACE. Composition of alleles with frequencies of 1% or higher that result from SPACE editing with 28 gRNAs. Data are taken from the first replicate obtained for each gRNA on-target experiment shown in FIG. 2.

FIG. 5. On-target C-to-R (A/G) and A-to-Y (C/T) editing of miniABEmax-V82G, Target-AID, and SPACE. Box and dot plots showing on-target DNA C-to-R (A/G) and A-to-Y (C/T) editing frequencies with 28 gRNAs (n=4). In box plots, the box spans the interquartile range (IQR) (first to third quartiles), the horizontal line shows the median (second quartile) and whiskers extend to ±1.5×IQR. Single dots represent all Cs or As across the entire protospacer for all four replicates at each genomic site. Data from the same experiment as shown in FIG. 2.

FIG. 6. On-target indel frequencies induced by miniABEmax-V82G, Target-AID, and SPACE. Dot plots showing on-target DNA indel frequencies with 28 gRNAs (n=4). Single dots represent individual replicates. Data from the same experiment as shown in FIG. 2.

FIG. 7. DNA off-target editing by SPACE. Heat maps showing A-to-G and C-to-T DNA off-target editing frequencies with gRNAs targeted to HEK sites 2-4, EMX1 site 1, or FANCF site 1 (n=4 independent replicates). Location in the protospacer is indicated at the bottom with 1 being the most PAM-distal position.

FIG. 8. On-target DNA editing of SPACE compared to co-expression of miniABEmax-V82G and Target-AID (ABE & CBE mix) at 28 genomic sites. Heat maps showing on-target DNA A-to-G and C-to-T editing frequencies with 28 gRNAs (n=4 independent replicates). Numbering at the bottom represents the position of the respective base in the protospacer sequence with 1 being the most PAM-distal location.

FIGS. 9A-B. On-target C-to-T, A-to-G and dual editing, and indel frequencies induced by co-expression of miniABEmax-V82G and Target-AID compared with SPACE. A, Bar and dot plots showing mean sum of allele frequencies of all edited alleles with A-to-G only, C-to-T only, and concurrent A-to-G and C-to-T editing with 28 gRNAs (n=4). Single dots represent individual replicates. Error bars represent the standard deviation (SD). Data are from the same experiment as shown in FIG. 8. B, Dot plots showing on-target DNA indel frequencies with 28 gRNAs (n=4). Single dots represent individual replicates. Data are from the same experiment as shown in FIG. 8.

FIG. 10. Allele frequency tables of DNA on-target editing by co-expression of miniABEmax-V82G and Target-AID (ABE & CBE mix) compared to expression of SPACE. Composition of alleles with frequencies of 1% or higher that result from co-expression of miniABEmax-V82G and Target-AID (ABE & CBE mix) or SPACE editing with 28 gRNAs. Data are taken from the first replicate obtained for each target site shown in FIG. 8. Numbering indicates the position in the spacer with 1 being the most PAM-distal location.

FIGS. 11A-B. RNA off-target editing profile of SPACE. A, Jitter plots show transcriptomic A-to-I and C-to-U mutations detected in RNA-seq experiments from HEK293T cells. GFP control cells express no gRNA. Data are shown from three independent replicates. n=number of combined adenines and cytosines modified. B, Histograms showing the total number of RNA A-to-1 or C-to-U edits observed (y-axis) with different editing efficiencies (x-axis). n=number of modified adenines and cytosines. Experiments were performed in triplicate (data are from the same experiments shown in A).

FIGS. 12A-B. Potential amino acid and codon modifications induced by SPACE. A, Circos plot showing amino acid changes that can be induced by SPACE (gray), including amino acid changes in specific codon context, that are uniquely enabled (black). B, Circos plot showing 60 unique codon changes (with respect to the start codon) that can be induced by dual editing of adenines and cytosines by SPACE (grey), 18 of which (black) lead to unique SPACE-inducible amino acid changes with respect to the original codon (also see Table D).

FIG. 13. SPACE can introduce saturated mutagenesis across a region of interest. Schematic illustration showing an example wherein SPACE can introduce A>G, T>C, C>T, and G>A for every nucleotide available across a coding or non-coding region to introduce saturation mutagenesis, e.g. in the context of a SPACE tiling screen.

### DETAILED DESCRIPTION

ABEs install A-to-G transitions in DNA while CBEs allow for the installation of C-to-T mutations. However, a certain subset of mutations (double or triple nucleotide variants), as well as amino acid mutations that cannot be targeted with available CBEs and ABEs (e.g., transversion mutations) evade current base editing strategies.

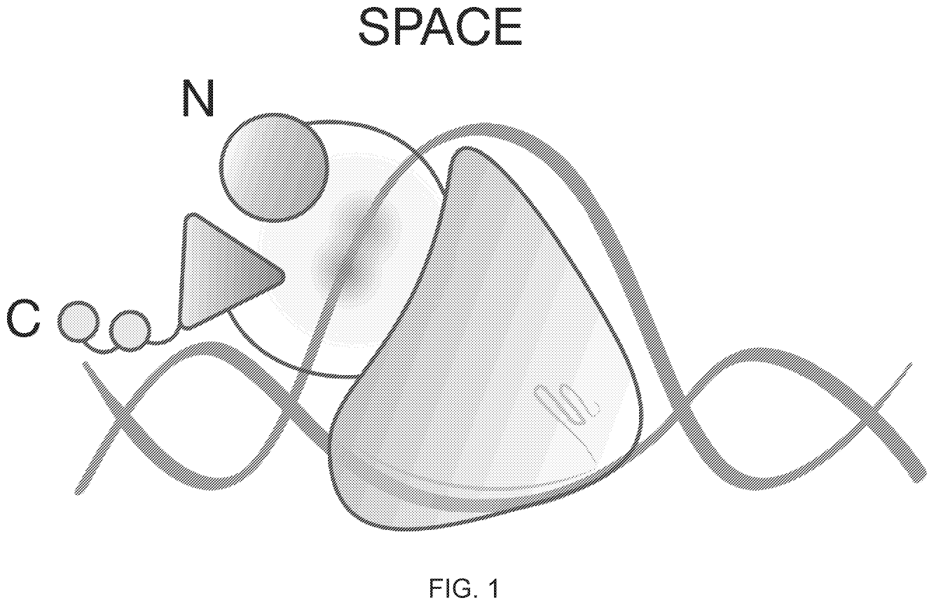

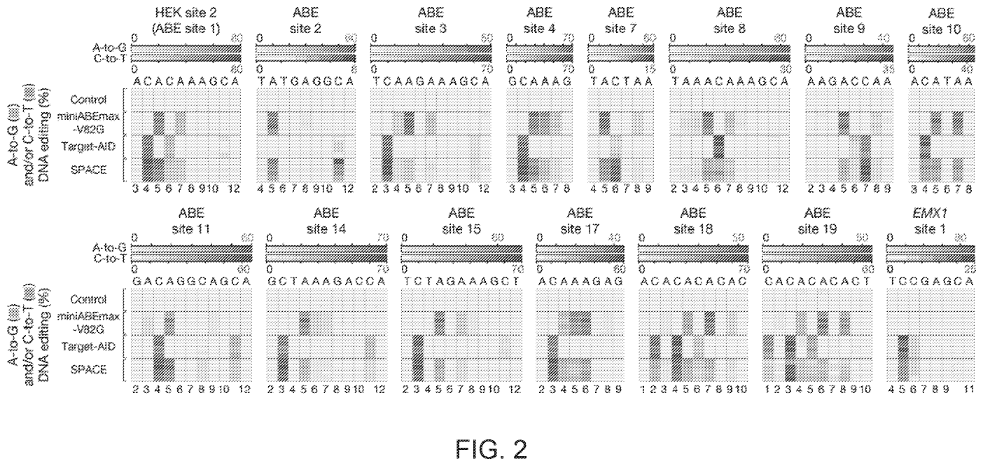

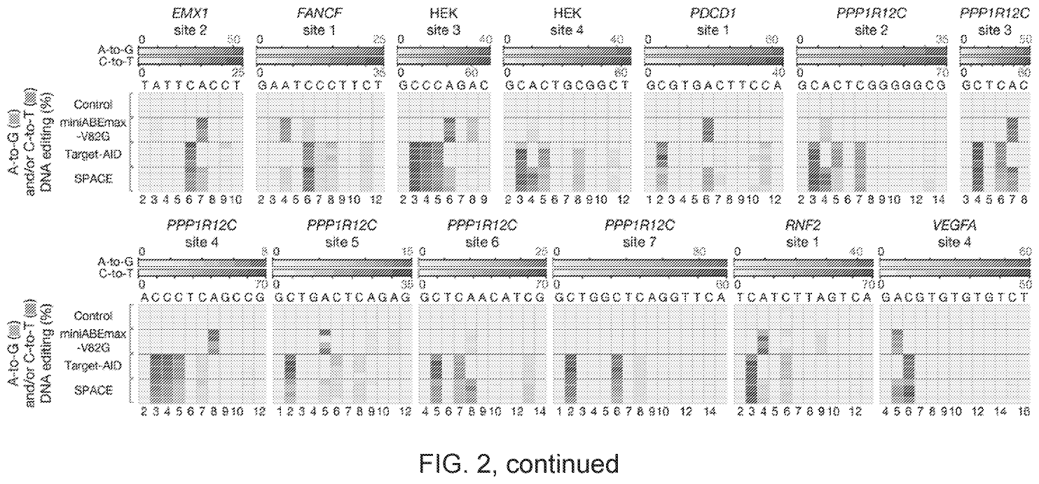

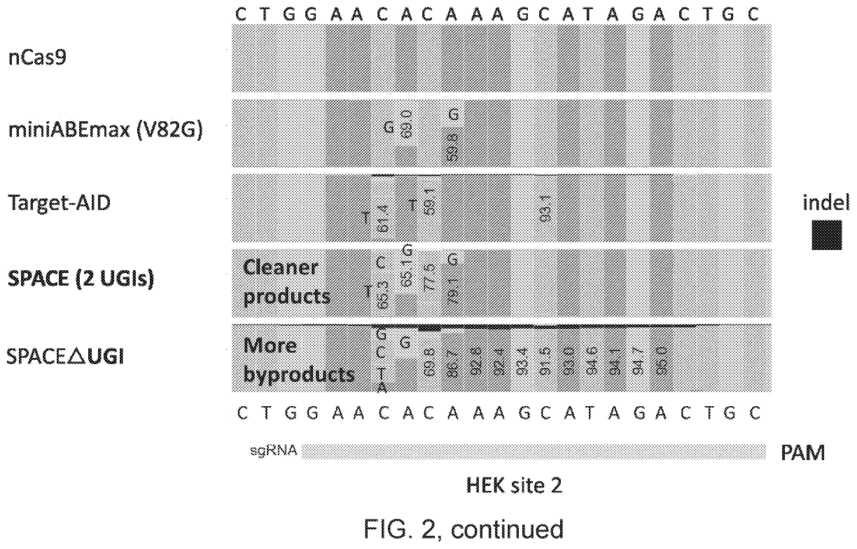

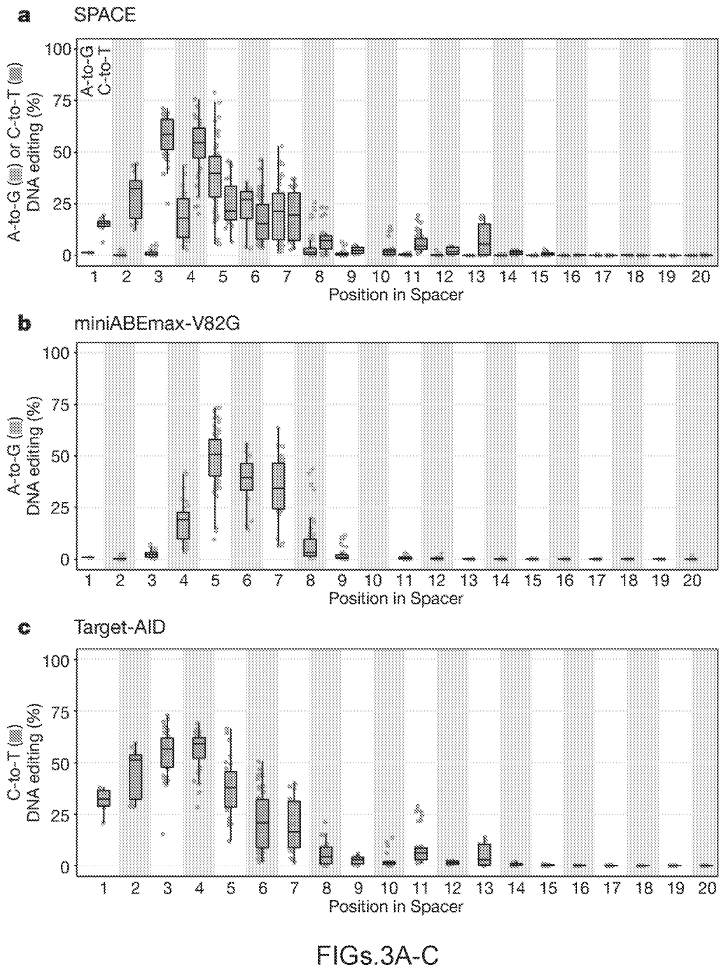

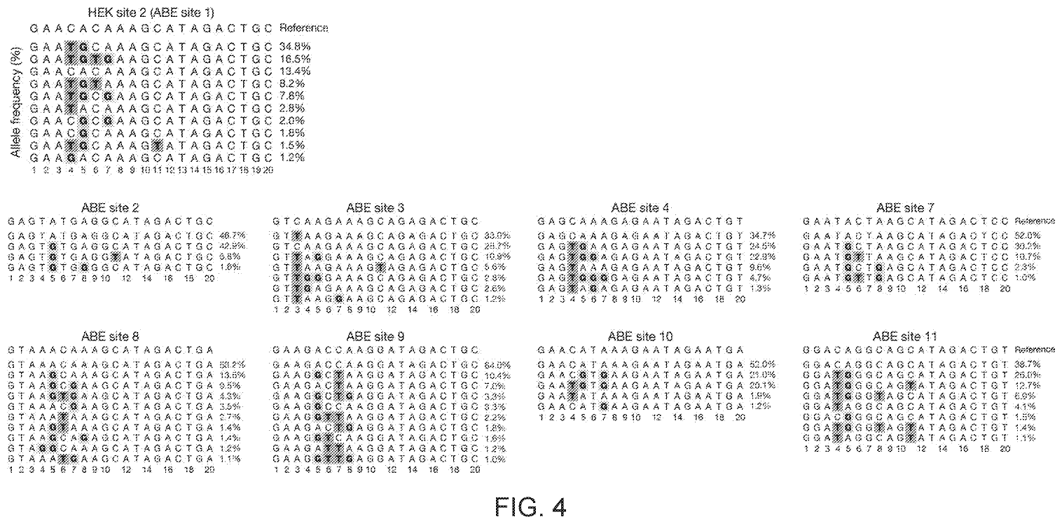

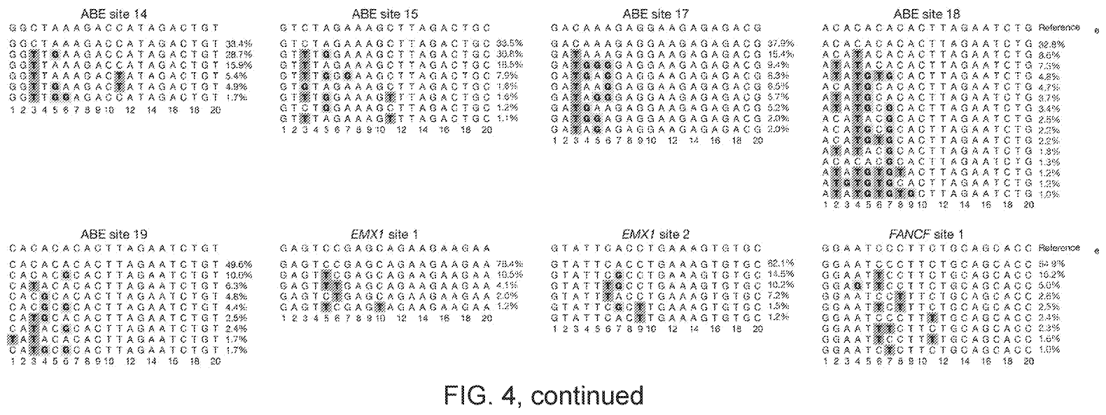

We sought to investigate if the combination of two separate deaminases in one BE architecture would allow for efficient parallel A-to-G and C-to-T editing in one editing window in cells treated with this bifunctional adenine and cytosine editor (BACE). In its first iteration, we fused a mutant E. coli TadA monomer (adenosine deaminase), previously described in the context of miniABEmax-V82G, as well as pmCDA1 (cytidine deaminase) and two UGIs to a catalytically impaired Cas9 (D10A) which resulted in the engineering of the SpCas9-based adenine and cytosine editor (SPACE, FIG. 1). Transfection of this editor into human HEK293T cells yielded efficient simultaneous deamination of cytosines and adenines in the same editing window at multiple sites that we tested in quadruplicate experiments (FIG. 2). The distribution of dual base edits across the protospacer induced by SPACE resembles the respective editing distributions of miniABEmax-V82G and Target-AID (FIG. 3). To test if the edits are present in the same allele, we analyzed the frequency of alleles that show both edits and saw that, e.g., for HEK site 2, there is a substantial fraction of alleles that show both edits (FIG. 4).

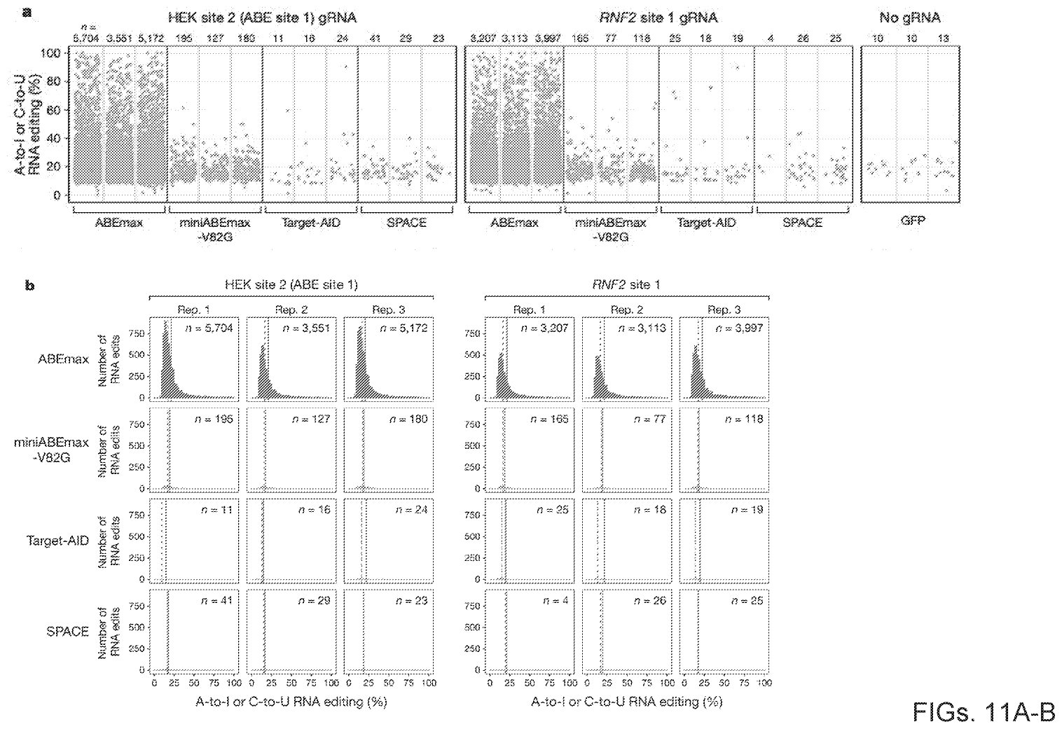

Both the adenosine deaminase and the cytidine deaminase domain used in the exemplary versions of SPACE described herein were chosen due to their relatively small size (e.g., TadA*-V82G monomer instead of TadA-TadA* heterodimer used in WT-ABE) and their dramatically reduced RNA off-target editing (Grunewald et al, Nature Biotechnology 2019; FIGS. 11A-B).

Thus, described herein are variants of base editor fusion proteins that enable the parallel installation of two distinct transition mutations (C-to-T and A-to-G) simultaneously in one editing window, by fusing two deaminase domains to one DNA-binding protein (Cas9 nickase) and one or multiple uracil-glycosylase inhibitor (UGI) proteins. BACE editing with SPACE enables this combinatorial editing without introducing two separate ABE and CBE base editors into the cell, enabling easier stochiometric delivery of the two deaminases, as well as Cas9 and UGI components as well as a smaller packaging size compared to the delivery of two separate BEs. Additionally, the possibility to install two substitutions side-by-side in one or multiple codons will enable more expanded in vitro and in vivo amino acid and protein modifications. A table of potentially targetable amino acid changes are shown in Table D and a list of potential disease targets (using Cas proteins compatible with NGG, NG, NAA, and NGA PAMs) is shown in Table E-K.

### Exemplary Adenosine Deaminase Domains Used for SPACE and BACE

In some embodiments, the adenosine deaminase is TadA from E. coli, or an orthologue from a different prokaryote, e.g., S. aureus, or a homologue from the eukaryotic domain, such as yeast TAD1/2 or a mammalian species such as human (e.g., ADAT2; Table C). The tRNA-specific adenosine deaminase family members have high sequence homology and many of these orthologues may be compatible with one or more of the amino acid substitutions in E. coli TadA expected to cause an RRE phenotype and would be desirable in a SPACE or BACE architecture.

The wild type sequence of wild type E. coli TadA, available in uniprot at P68398, is as follows:

(SEQ ID NO: 1)
MSEVEFSHEYWMRHALTLAKRAWDEREVPVGAVINHNNRVIGEGWNRPI
GRHDPTAHAEIMALRQGGLVMQNYRLIDATLYVTLEPCVMCAGAMIHSR
IGRVVFGARDAKTGAAGSLMDVLHHPGMNHRVEITEGILADECAALLSD
FFRMRRQEIKAQKKAQSSTD.

The engineered E. coli TadA sequence present in ABE7.10 and ABEmax is as follows (SEQ ID: 226):

SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD.

In the most commonly used ABEs (ABE7.10 and ABEmax), these two proteins are fused using a 32 amino acid linker (bolded in sequence below), forming a heterodimer, the sequence of which is as follows (SEQ ID: 227):

MSEVEFSHEYWMRHALTLAKRAWDEREVPVGAVELVHNNRVIGEGWNRP
IGRHDPTAHAEIMALRQGGLVMQNYRLIDATLYVTLEPCVMCAGAMIHS
RIGRVVFGARDAKTGAAGSLMDVLHHPGNANHRVEITEGILADECAALL
SDFFRMRRQEIKAQKKAQSSTDSGGSSGGSSGSETPGTSESATPESSGG
SSGGSSEVEFSHEYWMRHALTLAKRARDEREVPVGAVLVLNNRVIGEGW
NRAIGLHDPTAHAEIMALRQGGLVMQNYRLIDATLYVTFEPCVMCAGAM
IHSRIGRVVFGVRNAKTGAAGSLMDVLHYPGMNHRVEITEGILADECAA
LLCYFFRMPRQVFNAQKKAQSSTD.

Other exemplary sequences are shown in Table C. These tRNA-specific adenosine deaminase orthologues and homologues also represent candidates for inclusion of the mutations previously described at analogous positions in these proteins.

In some embodiments, the base editors include catalytically dead adenine deaminase variants, e.g., E59A. (Gaudelli et al, 2017, PMID: 29160308) as part of a heterodimer.

In some embodiments, the adenosine deaminase domain (monomeric or dimeric) from ABE8s (also with additional V106W mutation) could be used (Gaudelli et al, Nature Biotechnology 2020, PMID: 32284586; SEQ-IDs: 148-151).

In some embodiments, the adenosine deaminase domain (monomeric or dimeric) from ABE8e (with V82G, K20A/R21A, or V106W mutations) could be used (Richter et al, Nature Biotechnology 2020, DOI: https://doi.org/10.1038/s41587-020-0453-z; SEQ-IDs: 145-147).

### Exemplary Cytidine Deaminase Domains Used for SPACE and BACE

A number of cytidine deaminase domains that can be used in the proteins described herein are known in the art. In some embodiments, the cytidine deaminase is pmCDA1 (sea lamprey) or APOBEC1 from rat, or from a different species (Table A), e.g., a different mammalian species such as H. sapiens. The APOBEC, AICDA (AID) and CDA1 family members have high sequence homology and represent potential candidates for BACE and SPACE BE architectures (Table B)^{2, 10-13}.

Specifically, rAPOBEC1, enhanced human A3A, and human AID^{6, 12, 14} or pmCDA1² (Grünewald et al, Nature Biotechnology, in press, preprint on bioRxiv doi: https://doi.org/10.1101/631721) are candidates for inclusion into the SPACE and BACE architectures. Alternatively, the CBE variants FERNY and evoFERNY (Thuronyi et al, Nature Biotechnology 2019, PMID 31332326; SEQ-IDs: 152-153) as well as the CBE variants YE1, YE2, and YEE (Kim et al, Nature Biotechnology 2017, PMID 28191901; SEQ-IDs: 154-156) could be used to e.g., achieve increased cytosine deamination in a 5′G context (Thuronyi et al) or to achieve less DNA and RNA off-targets (Kim et al).

### Programmable DNA Binding Domains

In some embodiments, the base editors include programmable DNA binding domains such as engineered C2H2 zinc-fingers, transcription activator effector-like effectors (TALEs), and Clustered Regularly Interspaced Short Palindromic Repeats (CRISPR) Cas RNA-guided nucleases (RGNs) and their variants, including ssDNA nickases (nCas9) or their analogs and catalytically inactive dead Cas9 (dCas9) and its analogs (e.g., as shown in Table L), and any engineered protospacer-adjacent motif (PAM) or high-fidelity variants (e.g., as shown in Table M). A programmable DNA binding domain is one that can be engineered to bind to a selected target sequence. CRISPR-Cas Nucleases Although herein we refer to Cas9, in general any Cas9-like nickase could be used (including the related Cpf1/Cas12a enzyme classes), unless specifically indicated.

See, e.g., Tables L and M. These orthologs, and mutants and variants thereof as known in the art, can be used in any of the fusion proteins described herein. See, e.g., WO 2017/040348 (which describes variants of SaCas9 and SpCas 9 with increased specificity) and WO 2016/141224 (which describes variants of SaCas9 and SpCas 9 with altered PAM specificity).

The Cas9 nuclease from S. pyogenes (hereafter simply Cas9) can be guided via simple base pair complementarity between 17-20 nucleotides of an engineered guide RNA (gRNA), e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1, also known as Cas12a) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerdund et al., Genome Biol 16, 251 (2015). Unlike SpCas9, Cpf1/Cas12a requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCp1 recognize TTTN PAMs that are found 5′ of the protospacer (Id.).

In some embodiments, the present system utilizes a wild type or variant Cas9 protein from S. pyogenes or Staphylococcus aureus, or a wild type or variant Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., Nat Biotechnol. 2016 August; 34(8):869-74; Tsai and Joung, Nat Rev Genet. 2016 May; 17(5):300-12; Kleinstiver et al., Nature. 2016 Jan. 28; 529(7587):490-5; Shmakov et al., Mol Cell. 2015 Nov. 5; 60(3):385-97; Kleinstiver et al., Nat Biotechnol. 2015 December; 33(12):1293-1298; Dahlman et al., Nat Biotechnol. 2015 November; 33(11):1159-61; Kleinstiver et al., Nature. 2015 Jul. 23; 523(7561):481-5; Wyvekens et al., Hum Gene Ther. 2015 July; 26(7):425-31; Hwang et al., Methods Mol Biol. 2015; 1311:317-34; Osbom et al., Hum Gene Ther. 2015 February; 26(2):114-26; Konermann et al., Nature. 2015 Jan. 29; 517(7536):583-8; Fu et al., Methods Enzymol. 2014; 546:21-45; and Tsai et al., Nat Biotechnol. 2014 June; 32(6):569-76, inter alia. Concerning rAPOBEC1 itself, a number of variants have been described, e.g., Chen et al, RNA. 2010 May; 16(5):1040-52; Chester et al, EMBO J. 2003 Aug. 1; 22(15):3971-82: Teng et al, J Lipid Res. 1999 April; 40(4):623-35; Navaratnam et al, Cell. 1995 Apr. 21; 81(2):187-95; MacGinnitie et al, J Biol Chem. 1995 Jun. 16; 270(24):14768-75; Yamanaka et al, J Biol Chem. 1994 Aug. 26; 269(34):21725-34. The guide RNA is expressed or present in the cell together with the Cas9 or Cpf1. Either the guide RNA or the nuclease, or both, can be expressed transiently or stably in the cell or introduced as a purified protein or nucleic acid.

In some embodiments, the Cas9 also includes one of the following mutations, which reduce nuclease activity of the Cas9; e.g., for SpCas9, mutations at D10A or H840A (which creates a single-strand nickase).

In some embodiments, the SpCas9 variants also include mutations at one of each of the two sets of the following amino acid positions, which together destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432).

In some embodiments, the Cas9 is fused to one or more SV40 or bipartite (bp) nuclear localization sequences (NLSs) protein sequences; an exemplary (bp) NLS sequence is as follows: (KRTADGSEFES)PKKKRKV (SEQ ID NO: 24); others are known in the art and provided herein. Typically, the NLSs are at the N- and C-termini of a fusion protein, but can also be positioned at the N- or C-terminus, or between the DNA binding domain and the deaminase domain. Linkers as known in the art can be used to separate domains.

### TAL Effector Repeat Arrays

Transcription activator like effectors (TALEs) of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity may be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

TALE proteins may be useful in research and biotechnology as targeted chimeric nucleases that can facilitate homologous recombination in genome engineering (e.g., to add or enhance traits useful for biofuels or biorenewables in plants). These proteins also may be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in U.S. Ser. No. 61/610,212, and Reyon et al., Nature Biotechnology 30, 460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

### Zinc Fingers

Zinc finger (ZF) proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

One existing method for engineering zinc finger arrays, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

### Uracil Glycosylase Inhibitor (UGI)

In some embodiments, the DBD is fused to one or more Uracil glycosylase inhibitor (UGI) protein sequences; an exemplary UGI sequence is as follows:

(SEQ ID NO: 230; Uniprot: P14739)
TNLSDIIEKETGKQLVIQESILMLPEEVEEVIGNKPESDILVHTAYDES
TDENVMLLTSDAPEYKPWALVIQDSNGENKIKML,

Typically, the UGIs are at the C-terminus of a fusion protein as described herein, but can also be positioned at the N-terminus, or between the DNA binding domain and a deaminase domain. Linkers as known in the art can be used to separate domains.

### Variants

In some embodiments, the components of the fusion proteins are at least 80%, e.g., at least 85%, 90%, 95%, 97%, or 99% identical to the amino acid sequence of a exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains a desired activity of the parent, e.g., deaminase activity, and/or the ability to interact with a guide RNA and/or target DNA, optionally with improved specificity or altered substrate specificity.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™ Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Also provided herein are isolated nucleic acids encoding the base editor fusion proteins, vectors comprising the isolated nucleic acids, optionally operably linked to one or more regulatory domains for expressing the variant proteins, and host cells, e.g., mammalian host cells, comprising the nucleic acids, and optionally expressing the variant proteins. In some embodiments, the host cells are stem cells, e.g., hematopoietic stem cells.

In some embodiments, the fusion proteins include a linker between the DNA binding domain (e.g., ZFN, TALE, or nCas9) and the BE domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:135) or GGGGS (SEQ ID NO:136), e.g., two, three, four, or more repeats of the GGGS (SEQ ID NO:137) or GGGGS (SEQ ID NO:138) unit. Other linker sequences can also be used.

In some embodiments, the deaminase fusion protein includes a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g., the mitochondria and the nucleus. Examples of molecules that can be delivered by CPPs include therapeutic drugs, plasmid DNA, oligonudeotides, siRNA, peptide-nucleic acid (PNA), proteins, peptides, nanopartides, and liposomes. CPPs are generally 30 amino acids or less, are derived from naturally or non-naturally occurring protein or chimeric sequences, and contain either a high relative abundance of positively charged amino acids, e.g., lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g., chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

Alternatively or in addition, the deaminase fusion proteins can include a nuclear localization sequence, e.g., SV40 large T antigen NLS (PKKKRRV (SEQ ID NO:221)); nucleoplasmin NLS (KRPAATKKAGQAKKKK (SEQ ID NO:222); or bipartite NLS (KRTADGSEFEPKKKRKV (SEQ ID NO:229); or (KRTADGSEFES)PKKKRKV (SEQ ID NO: 24). Other NLSs are known in the art; see, e.g., Cokol et al., EMBO Rep. 2000 Nov. 15; 1(5): 411-415; Freitas and Cunha, Curr Genomics. 2009 December; 10(8): 550-557.

In some embodiments, the deaminase fusion proteins include a moiety that has a high affinity for a ligand, for example GST, FLAG or hexahistidine sequences. Such affinity tags can facilitate the purification of recombinant deaminase fusion proteins.

The deaminase fusion proteins described herein can be used for altering the genome of a cell. The methods generally include expressing or contacting the deaminase fusion proteins in the cells; in versions using one or two Cas9s, the methods include using a guide RNA having a region complementary to a selected portion of the genome of the cell. Methods for selectively altering the genome of a cell are known in the art, see, e.g., U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US20160024529; US20160024524; US20160024523; US20160024510; US20160017366; US20160017301; US20150376652; US20150356239; US20150315576; US20150291965; US20150252358; US20150247150; US20150232883; US20150232882; US20150203872; US20150191744; US20150184139; US20150176064; US20150167000; US20150166969; US20150159175; US20150159174; US20150093473; US20150079681; US20150067922; US20150056629; US20150044772; US20150024500; US20150024499; US20150020223; US20140356867; US20140295557; US20140273235; US20140273226; US20140273037; US20140189896; US20140113376; US20140093941; US20130330778; US20130288251; US20120088676; US20110300538; US20110236530; US20110217739; US20110002889; US20100076057; US20110189776; US20110223638; US20130130248; US20150050699; US20150071899; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; WO/2008/108989; WO/2010/054108; WO/2012/164565; WO/2013/098244; WO/2013/176772; US 20150071899; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; AI-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

For methods in which the deaminase fusion proteins are delivered to cells, the proteins can be produced using any method known in the art, e.g., by in vitro translation, or expression in a suitable host cell from nucleic acid encoding the deaminase fusion protein; a number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the deaminase fusion proteins can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1):180-194.

### Expression Systems

To use the deaminase fusion proteins described herein, it may be desirable to express them from a nucleic acid that encodes them. This can be performed in a variety of ways. For example, the nucleic acid encoding the deaminase fusion can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the deaminase fusion for production of the deaminase fusion protein. The nucleic acid encoding the deaminase fusion protein can also be cloned into an expression vector, for administration to a plant cell, animal cell, preferably a mammalian cell or a human cell, fungal cell, bacterial cell, or protozoan cell. To obtain expression, a sequence encoding a deaminase fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the deaminase fusion protein is to be administered in vivo for gene regulation, either a constitutive or an inducible promoter can be used, depending on the particular use of the deaminase fusion protein. In addition, a preferred promoter for administration of the deaminase fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the deaminase fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the deaminase fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the deaminase fusion protein can include RNA Pol III promoters to drive expression of the guide RNAs, e.g., the H1, U6 or 7SK promoters. These human promoters allow for expression of deaminase fusion protein in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the deaminase fusion protein.

In methods wherein the fusion proteins include a Cas9 domain, the methods also include delivering at least one gRNA that interacts with the Cas9, or a nucleic acid that encodes a gRNA.

Alternatively, the methods can include delivering the deaminase fusion protein and guide RNA together, e.g., as a complex. For example, the deaminase fusion protein and gRNA can be can be overexpressed in a host cell and purified, then complexed with the guide RNA (e.g., in a test tube) to form a ribonudeoprotein (RNP), and delivered to cells. In some embodiments, the deaminase fusion protein can be expressed in and purified from bacteria through the use of bacterial expression plasmids. For example, His-tagged deaminase fusion protein can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

The present invention also includes the vectors and cells comprising the vectors, as well as kits comprising the proteins and nucleic acids described herein, e.g., for use in a method described herein.

### Methods of Use

The base editors described herein can be used to deaminate a selected adenine and/or cytosine, enabling the parallel installation of two distinct transition mutations (C-to-T and A-to-G) simultaneously in one editing window, in a nucleic acid sequence, e.g., in a cell, e.g., a plant, bacterial, fungal, or animal cell. The cell can be isolated (e.g., ex vivo or in vitro) or in an animal (e.g., a mammal such as a human or veterinary subject), or a synthetic nucleic acid substrate, e.g., in vitro. The nucleic acid sequence can be, e.g., genomic DNA or mitochondrial DNA. The methods include contacting the nucleic acid with a BACE as described herein; in some embodiments the methods can be used to induce a change as shown in Table D. Where the base editor includes a CRISPR Cas9 or Cas12a protein, the methods further include the use of one or more guide RNAs (gRNAs) that direct binding of the base editor to a sequence to be deaminated. The target sequence (editing window) is located on the non-target strand (NTS), so it's the ssDNA strand that's opened up when Cas9 opens up DNA, but it's not bound by the gRNA. The NTS protospacer sequence in the target organism's DNA has the same sequence as the gRNA's spacer sequence (for both base and prime editing). Thus when SPACE is used, the gRNA protospacer directs the base editor to the target sequence, preferably wherein the target sequence comprises a cytosine to be edited at one of positions 2-7, or an adenine to be edited at one of positions 4-7.

For example, the base editors described herein can be used for in vitro, in vivo or in situ directed evolution, e.g., to engineer polypeptides or proteins based on a synthetic selection framework, e.g., antibiotic resistance in E. coli or resistance to anti-cancer therapeutics being assayed in mammalian cells (e.g., CRISPR-X Hess et al, Nat Methods. 2016 December; 13(12):1036-1042, or BE-plus systems Jiang et al, Cell Res. 2018 August; 28(8):855-861). The BACEs described herein can also be used, e.g., for targeted sequence diversification.

In some embodiments, the BACEs can be used to correct or alter a disease-causing mutation, or to introduce a protective mutation, in a cell, e.g., in a human cell, e.g., in vitro/ex vivo or in vivo; exemplary mutations can include those listed in Table E. When the alteration is made ex vivo, the edited cell can then be re-introduced into the subject. These methods can be used to treat, reduce risk of developing, delay onset of, or ameliorate a disease, e.g., a disease listed in Table E.

The BACEs can also be used to generate a cell or animal model by introducing a mutation, e.g., a disease-causing mutation, e.g., a multinucleotide variant (MNV, i.e., a variant found in phase with another variant), e.g., a MNV mutation as listed in Tables F-K. See, e.g., Example 9.

BACE/SPACE could be used for introducing A>G, T>C (A>G on the other strand), C>T, or G>A (C>T on the other strand) for every nucleotide available across a coding/non-coding region to generate a comprehensive library. This can enable high-throughput saturation mutagenesis screening and highly complex genotype-phenotype correlation to study a protein or gene of interest.

These methods can be used for generating two or more sets of nucleic acids, each set comprising a plurality of sequences, wherein each set comprises one or more nucleic acids having the same sequence, and wherein each set differs from each of the other sets by at least one nucleotide. These methods include (i) providing a first nucleic acid comprising a first sequence, e.g., a reference or wild type sequence; (ii) contacting the first nucleic acids with a BACE as described herein, wherein the programmable DNA binding domain is a CRISPR Cas RGN or a variant thereof; and a least one guide RNA compatible with the base editor that directs the base editor to modify a selected nucleotide in the first sequence; and (iii) isolating a second nucleic acid comprising a sequence comprising the selected modification in the nucleotide sequence, to provide a second set of nucleic acids. The methods can include amplifying the second nucleic acids. Steps (i)-(iii) can be repeated until a desired number of sets is obtained, e.g., until enough sets are obtained to include at least one set with a mutation at each position in a selection region of the sequence of the nucleic acid.

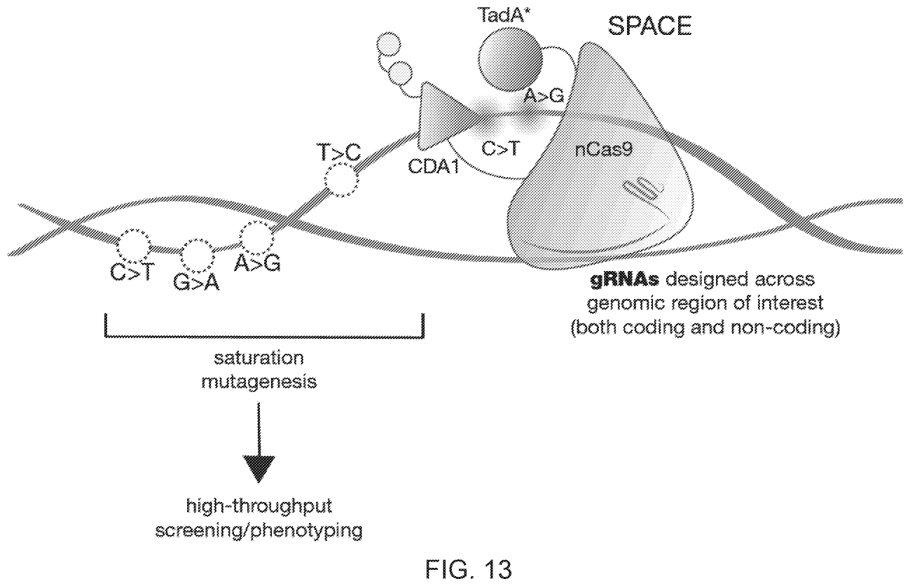

In some embodiments, each separate set of variant is expressed in a separate organism, and effects on phenotype can be evaluated, e.g., for programmable sequence diversification. As one example, the methods can be used to develop a plant with a desired characteristic (e.g., early harvest, pest resistance, drought tolerance, taste, sweetness, storage, resistance to browning). The methods can be used to mutate a region in a specific gene, e.g., to shuffle the region, to produce a number of variant plants. The plants can then be grown, and effects on the desired characteristic evaluated and selected. See, e.g., Li et al., Nat Biotechnol (2020). doi.org/10.1038/s41587-019-0393-7; FIG. 13 and Example 10.

### TABLES

TABLE A

| | | Uniprot | | Seq |
|---|---|---|---|---|
| APOBEC1 | | accession | | ID |
| orthologue | Species | number | Version number | NO: |
| African | Loxodonta | G3U0R4 | version 30 of the | 1 |
| elephant | africana | | entry and version 1 of | |
| | | | the sequence | |
| African lungfish | Protopterus | A0A0M3N0G8 | version 4 of the entry | 2 |
| | annectens | | and version 1 of the | |
| | | | sequence | |
| American | Alligator | A0A151P6M4 | version 9 of the entry | 3 |
| alligator | mississippiensis | | and version 1 of the | |
| | | | sequence | |
| American | Anolis | F1CGT0 | version 16 of the | 4 |
| chameleon | carolinensis | | entry and version 1 of | |
| | | | the sequence | |
| American crow | Corvus | A0A091EQ78 | version 8 of the entry | 5 |
| | brachyrhynchos | | and version 1 of the | |
| | | | sequence | |
| Anna's | Calypte anna | A0A091IIG0 | version 9 of the entry | 6 |
| hummingbird | | | and version 1 of the | |
| | | | sequence | |
| Atlantic | Tursiops | A0A2U4ALA1 | version 2 of the entry | 7 |
| bottle-nosed | truncatus | | and version 1 of the | |
| dolphin | | | sequence | |
| Barn owl | Tyto alba | A0A093FY71 | version 6 of the entry | 8 |
| | | | and version 1 of the | |
| | | | sequence | |
| Black flying | Pteropus alecto | L5KGJ8 | version 13 of the | 9 |
| fox | | | entry and version 1 of | |
| | | | the sequence | |
| Black | Rhinopithecus | A0A2K6KS69 | version 5 of the entry | 10 |
| snub-nosed | bieti | | and version 1 of the | |
| monkey | | | sequence | |
| Beluga whale | Delphinapterus | A0A2Y9NGP5 | version 1 of the entry | 11 |
| | leucas | | and version 1 of the | |
| | | | sequence | |
| Bengalese | Lonchura | A0A218ULD2 | version 3 of the entry | 12 |
| finch | striata | | and version 1 of the | |
| | domestica | | sequence | |
| Blue-fronted | Amazona | A0A0Q3WRD0 | version 5 of the entry | 13 |
| Amazon parrot | aestiva | | and version 1 of the | |
| | | | sequence | |
| Bolivian | Saimiri | A0A2K6U925 | version 5 of the entry | 14 |
| squirrel | boliviensis | | and version 1 of the | |
| monkey | boliviensis | | sequence | |
| Bonobo | Pan paniscus | A0A2R9A0R0 | version 2 of the entry | 15 |
| | | | and version 1 of the | |
| | | | sequence | |
| Bornean | Pongo | Q694B3 | version 60 of the | 16 |
| orangutan | pygmaeus | | entry and version 2 of | |
| | | | the sequence | |
| Bovine | Bos taurus | E1BP99 | version 40 of the | 17 |
| | | | entry and version 1 of | |
| | | | the sequence | |
| Brandt's bat | Myotis brandtii | S7PYX0 | version 9 of the entry | 18 |
| | | | and version 1 of the | |
| | | | sequence | |
| Cat | Felis catus | M3WB96 | version 31 of the | 19 |
| | | | entry and version 2 of | |
| | | | the sequence | |
| Cebus | Cebus | A0A2K5PZC0 | version 5 of the entry | 20 |
| capucinus | capucinus | | and version 1 of the | |
| imitator | imitator | | sequence | |
| Chimpanzee | Pan troglodytes | H2Q5C6 | version 32 of the | 21 |
| | | | entry and version 1 of | |
| | | | the sequence | |
| Chinese | Alligator | A0A1U7S7K7 | version 5 of the entry | 22 |
| alligator | sinensis | | and version 1 of the | |
| | | | sequence | |
| Chinese | Cricetulus | G3I1S7 | version 15 of the | 23 |
| hamster | griseus | | entry and version 1 of | |
| | | | the sequence | |
| Chuck-will's-widow | Antrostomus | A0A094MFH1 | version 10 of the | 24 |
| | carolinensis | | entry and version 1 of | |
| | | | the sequence | |
| Coquerel's | Propithecus | A0A2K6EVT9 | version 5 of the entry | 25 |
| sifaka | coquereli | | and version 1 of the | |
| | | | sequence | |
| Crab-eating | Macaca | G8F4P7 | version 11 of the | 26 |
| macaque | fascicularis | | entry and version 1 of | |
| | | | the sequence | |
| Crested ibis | Nipponia | A0A091V7F8 | version 9 of the entry | 27 |
| | nippon | | and version 1 of the | |
| | | | sequence | |
| Dalmatian | Pelecanus | A0A091SSF0 | version 8 of the entry | 28 |
| pelican | crispus | | and version 1 of the | |
| | | | sequence | |
| Damaraland | Fukomys | A0A091CVE5 | version 9 of the entry | 29 |
| mole rat | damarensis | | and version 1 of the | |
| | | | sequence | |
| David's myotis | Myotis davidii | L5LUG3 | version 11 of the | 30 |
| | | | entry and version 1 of | |
| | | | the sequence | |
| Dog | Canis lupus | F1PUJ5 | version 41 of the | 31 |
| | familiaris | | entry and version 2 of | |
| | | | the sequence | |
| Downy | Dryobates | A0A093GVH6 | version 9 of the entry | 32 |
| woodpecker | pubescens | | and version 1 of the | |
| | | | sequence | |
| Drill | Mandrillus | A0A2K5Z8Y4 | version 4 of the entry | 33 |
| | leucophaeus | | and version 1 of the | |
| | | | sequence | |
| East | Balearica | A0A087VMP5 | version 8 of the entry | 34 |
| African grey | regulorum | | and version 1 of the | |
| crowned-crane | gibbericeps | | sequence | |
| Emperor | Aptenodytes | A0A087QNJ5 | version 8 of the entry | 35 |
| penguin | forsteri | | and version 1 of the | |
| | | | sequence | |
| Enhydra lutris | Enhydra lutris | A0A2Y9IYV0 | version 1 of the entry | 36 |
| kenyoni | kenyoni | | and version 1 of the | |
| | | | sequence | |
| European | Mustela | B2NIW5 | version 34 of the | 37 |
| domestic ferret | putorius furo | | entry and version 1 of | |
| | | | the sequence | |
| Florida | Trichechus | A0A2Y9E587 | version 1 of the entry | 38 |
| manatee | manatus | | and version 1 of the | |
| | latirostris | | sequence | |
| Giant panda | Ailuropoda | G1LKL4 | version 27 of the | 39 |
| | melanoleuca | | entry and version 1 of | |
| | | | the sequence | |
| Golden-collared | Manacus | A0A093PWR2 | version 8 of the entry | 40 |
| manakin | vitellinus | | and version 1 of the | |
| | | | sequence | |
| Golden | Mesocricetus | Q9EQP0 | version 73 of the | 41 |
| hamster | auratus | | entry and version 1 of | |
| | | | the sequence | |
| Golden | Rhinopithecus | A0A2K6PRF3 | version 4 of the entry | 42 |
| snub-nosed | roxellana | | and version 1 of the | |
| monkey | | | sequence | |
| Green monkey | Chlorocebus | A0A0D9RBS4 | version 11 of the | 43 |
| | sabaeus | | entry and version 1 of | |
| | | | the sequence | |
| Guinea pig | Cavia porcellus | A0A286XNR2 | version 5 of the entry | 44 |
| | | | and version 1 of the | |
| | | | sequence | |
| Hawaiian monk | Neomonachus | A0A2Y9HAT6 | version 1 of the entry | 45 |
| seal | schauinslandi | | and version 1 of the | |
| | | | sequence | |
| Hoatzin | Opisthocomus | A0A091XJL0 | version 8 of the entry | 46 |
| | hoazin | | and version 1 of the | |
| | | | sequence | |
| Horse | Equus ferus | F6WR88 | version 28 of the | 47 |
| | caballus | | entry and version 1 of | |
| | | | the sequence | |
| Human | Homo sapiens | P41238 | version 166 of the | 48 |
| | | | entry and version 3 of | |
| | | | the sequence | |
| Kea | Nestor notabilis | A0A091RU17 | version 8 of the entry | 49 |
| | | | and version 1 of the | |
| | | | sequence | |
| Little egret | Egretta garzetta | A0A091IWL9 | version 10 of the | 50 |
| | | | entry and version 1 of | |
| | | | the sequence | |
| Ma's night | Aotus | A0A2K5DG70 | version 6 of the entry | 51 |
| monkey | nancymaae | | and version 1 of the | |
| | | | sequence | |
| Mouse | Mus musculus | P51908 | version 150 of the | 52 |
| | | | entry and version 1 of | |
| | | | the sequence | |
| Naked mole rat | Heterocephalus | G5BPM8 | version 16 of the | 53 |
| | glaber | | entry and version 1 of | |
| | | | the sequence | |
| Northern | Merops nubicus | A0A091QEK6 | version 8 of the entry | 54 |
| carmine | | | and version 1 of the | |
| bee-eater | | | sequence | |
| Northern | Fulmarus | A0A093LP85 | version 9 of the entry | 55 |
| fulmar | glacialis | | and version 1 of the | |
| | | | sequence | |
| Northern | Nomascus | G1QZV0 | version 31 of the | 56 |
| white-cheeked | leucogenys | | entry and version 1 of | |
| gibbon | | | the sequence | |
| Olive baboon | Papio anubis | A0A096MWB4 | version 19 of the | 57 |
| | | | entry and version 2 of | |
| | | | the sequence | |
| Gray | Monodelphis | Q9TUI7 | version 101 of the | 58 |
| short-tailed | domestica | | entry and version 1 of | |
| Opossum | | | the sequence | |
| Ord's kangaroo | Dipodomys ordii | A0A1S3FTE2 | version 3 of the entry | 59 |
| rat | | | and version 1 of the | |
| | | | sequence | |
| Pacific walrus | Odobenus | A0A2U3WPA5 | version 2 of the entry | 60 |
| | rosmarus | | and version 1 of the | |
| | divergens | | sequence | |
| Patagioenas | Patagioenas | A0A1V4JAP2 | version 3 of the entry | 61 |
| fasciata monilis | fasciata monilis | | and version 1 of the | |
| | | | sequence | |
| Peters' Angolan | Colobus | A0A2K5JKV4 | version 4 of the entry | 62 |
| colobus | angolensis | | and version 1 of the | |
| | palliatus | | sequence | |
| Philippine | Tarsius syrichta | A0A1U7U8J6 | version 3 of the entry | 63 |
| tarsier | | | and version 1 of the | |
| | | | sequence | |
| Pig | Sus scrofa | F1SLW4 | version 37 of the | 64 |
| | | | entry and version 2 of | |
| | | | the sequence | |
| Pig-tailed | Macaca | A0A2K6BGI5 | version 4 of the entry | 65 |
| macaque | nemestrina | | and version 1 of the | |
| | | | sequence | |
| Rabbit | Oryctolagus | P47855 | version 96 of the | 66 |
| | cuniculus | | entry and version 1 of | |
| | | | the sequence | |
| Rat | Rattus | P38483 | version 137 of the | 67 |
| | norvegicus | | entry and version 1 of | |
| | | | the sequence | |
| Red-legged | Cariama | A0A091M4D7 | version 10 of the | 68 |
| seriema | cristata | | entry and version 1 of | |
| | | | the sequence | |
| Red throated | Gavia stellata | A0A093F3R4 | version 8 of the entry | 69 |
| diver | | | and version 1 of the | |
| | | | sequence | |
| Rhesus | Macaca mulatta | G7N5W0 | version 19 of the | 70 |
| macaque | | | entry and version 1 of | |
| | | | the sequence | |
| Rifleman | Acanthisitta | A0A091MEP8 | version 8 of the entry | 71 |
| (Acanthisitta | chloris | | and version 1 of the | |
| chloris) | | | sequence | |
| Rock dove | Columba livia | A0A2I0LXZ8 | version 3 of the entry | 72 |
| | | | and version 1 of the | |
| | | | sequence | |
| Sheep | Ovis aries | W5NVH9 | version 19 of the | 73 |
| | | | entry and version 1 of | |
| | | | the sequence | |
| Small-eared | Otolemur | H0XVG8 | version 27 of the | 74 |
| galago | garnettii | | entry and version 1 of | |
| (Garnett's greater | | | the sequence | |
| bushbaby) | | | | |
| Smooth | Stylophora | A0A2B4RXQ3 | version 4 of the entry | 75 |
| cauliflower | pistillata | | and version 1 of the | |
| coral | | | sequence | |
| Sooty | Cercocebus | A0A2K5L2J6 | version 5 of the entry | 76 |
| mangabey | atys | | and version 1 of the | |
| | | | sequence | |
| Sperm whale | Physeter | A0A2Y9T649 | version 1 of the entry | 77 |
| | macrocephalus | | and version 1 of the | |
| | | | sequence | |
| Sumatran | Pongo abelii | H2NGD0 | version 24 of the | 78 |
| orangutan | | | entry and version 1 of | |
| | | | the sequence. | |
| Sunbittern | Eurypyga helias | A0A093JI54 | version 8 of the entry | 79 |
| | | | and version 1 of the | |
| | | | sequence | |
| Tasmanian | Sarcophilus | G3W4I1 | version 32 of the | 80 |
| devil | harrisii | | entry and version 1 of | |
| | | | the sequence | |
| Weddell seal | Leptonychotes | A0A2U3Y3M5 | version 2 of the entry | 81 |
| | weddellii | | and version 1 of the | |
| | | | sequence | |
| Western | Erinaceus | A0A1S3AN78 | version 3 of the entry | 82 |
| European | europaeus | | and version 1 of the | |
| hedgehog | | | sequence | |
| White-tailed | Haliaeetus | A0A091PSV3 | version 8 of the entry | 83 |
| sea-eagle | albicilla | | and version 1 of the | |
| | | | sequence | |
| White tufted | Callithrix | F7F6M6 | version 31 of the | 84 |
| ear marmoset | jacchus | | entry and version 2 of | |
| | | | the sequence | |
| Wild yak | Bos mutus | L8IDZ0 | version 15 of the | 85 |
| | | | entry and version 1 of | |
| | | | the sequence | |
| Yellow-throated | Pterocles | A0A093CIQ8 | version 5 of the entry | 86 |
| sandgrouse | gutturalis | | and version 1 of the | |
| | | | sequence | |

TABLE B
Exemplary APOBEC/AID family proteins. The following table lists
(in alphabetical order) exemplary APOBEC family homologues.

| APOBEC/AID | Uniprot | | |
|---|---|---|---|
| family | accession | | Seq. |
| homologue | number | Version number | ID |
| Rat APOBEC1 | P38483 | version 137 of the entry | 67 |
| | | and version 1 of the | |
| | | sequence | |
| Human AID | Q9GZX7 | version 155 of the entry | 87 |
| (AICDA) | | and version 1 of the | |
| | | sequence | |
| Human | P41238 | version 166 of the entry | 48 |
| APOBEC1 | | and version 3 of the | |
| | | sequence | |
| Human | Q9Y235 | version 132 of the entry | 88 |
| APOBEC2 | | and version 1 of the | |
| | | sequence | |
| Human | P31941 | version 160 of the entry | 89 |
| APOBEC3A | | and version 3 of the | |
| | | sequence | |
| Human | Q9UH17 | version 150 of the entry | 90 |
| APOBEC3B | | and version 1 of the | |
| | | sequence | |
| Human | Q9NRW3 | version 147 of the entry | 91 |
| APOBEC3C | | and version 2 of the | |
| | | sequence | |
| Human | Q96AK3 | version 127 of the entry | 92 |
| APOBEC3D | | and version 1 of the | |
| | | sequence | |
| Human | Q8IUX4 | version 143 of the entry | 93 |
| APOBEC3F | | and version 3 of the | |
| | | sequence | |
| Human | Q9HC16 | version 168 of the entry | 94 |
| APOBEC3G | | and version 1 of the | |
| | | sequence | |
| Human | Q6NTF7 | version 115 of the entry | 95 |
| APOBEC3H | | and version 4 of the | |
| | | sequence | |
| Petromyzon | NCBI Genbank: | Version 1 of the entry, | 96 |
| marinus cytosine | ABO15149.1 | accession EF094822.1 | |
| deaminase | Uniprot: A5H718 | | |
| (pmCDA1) | | | |
| Petromyzon | Same sequence | — | 97 |
| marinus cytosine | as ID 96, but | | |
| deaminase | with R187W | | |
| (pmCDA1) | mutation | | |

TABLE C
Exemplary TadA proteins. Some or all residues listed
in Table A as well as combinations thereof might
also be introduced in any of these TadA orthologues
or tRNA adenosine deaminase homologues (see FIG.
5 for alignments of these TadA proteins).

| tRNA-specific | Uniprot | | |
|---|---|---|---|
| adenosine | accession | Sequence | Seq. |
| deaminase | number | version # | ID |
| E. coli TadA | P68398 | 2 | 98 |
| S. aureus TadA | Q99W51 | 1 | 99 |
| S. pyogenes TadA | Q5XE14 | 2 | 100 |
| S. typhi TadA | Q8XGY4 | 2 | 101 |
| A. aeolicus TadA | O67050 | 1 | 102 |
| S. pombe TAD2 | O94642 | 2 | 103 |
| S. cerevisiae TAD1 | P53065 | 1 | 104 |
| S. cerevisiae TAD2 | P47058 | 1 | 105 |
| A. thaliana TAD2 | Q6IDB6 | 1 | 106 |
| X. laevis ADAT2 | Q4V7V8 | 1 | 107 |
| X. tropicalis ADAT2 | Q0P4H0 | 1 | 108 |
| D. rerio ADAT2 | Q5RIV4 | 2 | 109 |
| B. taurus ADAT2 | Q5E9J7 | 1 | 110 |
| M. musculus ADAT2 | Q6P6J0 | 1 | 111 |
| H. sapiens ADAT2 | Q7Z6V5 | 1 | 112 |

TABLE D
Unique codon and amino acid changes inducible with SPACE
compared to those by ABE or CBE alone. Listing potential codon changes, as
well as amino acid modifications that can be induced by CBE, ABE, and SPACE.

| | | Codon | AA | Codon | AA | Codon | AA | |
|---|---|---|---|---|---|---|---|---|
| WT | WT | mutations | mutations | mutations | mutations | mutations | mutations | |
| codon | AA | by ABE | by ABE | by CBE | by CBE | by SPACE | by SPACE | |
| AAC | N | GGC, GAC, | N > G, | AAT | = | GGT, AAT, | N > G, = , | A > G |
| | | AGC | N > D, N > S | | | GAC, AGC, | N > D, N > S | and |
| | | | | | | GGC, GAT, | | C > T |
| | | | | | | AGT | | |
| ACA | T | GCG, GCA, | T > A, = | ATA | T > I | 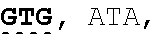 | 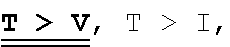 | |
| | | ACG | | | | GCA, ACG, | 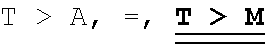 | |
| | | | | | | 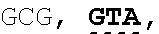 | | |
| | | | | | | 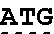 | | |
| ACC | T | GCC | T > A | ATT, ATC, | T > I, = | 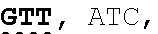 | 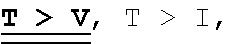 | |
| | | | | ACT | | GCC, ACT, | T > A, = | |
| | | | | | | 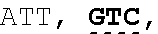 | | |
| | | | | | | GCT | | |
| ACG | T | GCG | T > A | ATG | T > M | 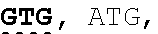 | 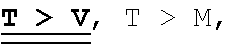 | |
| | | | | | | GCG | T > A | |
| ACT | T | GCT | T > A | ATT | T > I | 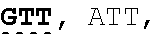 | 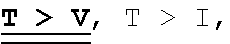 | |
| | | | | | | GCT | T > A | |
| AGC | S | GGC | S > G | AGT | = | GGT, AGT, | S > G, = | |
| | | | | | | GGC | | |
| ATC | I | GTC | I > V | ATT | = | GTT, ATT, | I > V, = | |
| | | | | | | GTC | | |
| CA | Q | CGG, CGA, | Q > R, = | TAA | Q > STOP | 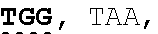 | 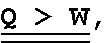 | |
| | | CAG | | | | CGA, CAG, | Q > STOP, | |
| | | | | | | CGG, TGA, | Q > R, = | |
| | | | | | | TAG | | |
| CAC | H | CGC | H > R | TAT, TAC, | H > Y, = | 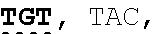 | 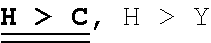 | |
| | | | | CAT | | CGC, CAT, | H > R, = | |
| | | | | | | 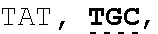 | | |
| | | | | | | CGT | | |
| CAG | Q | CGG | Q > R | TAG | Q > STOP | 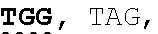 | 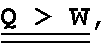 | |
| | | | | | | CGG | Q > STOP, | |
| | | | | | | | Q > R | |
| CAT | H | CGT | H > R | TAT | H > Y | 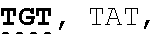 | 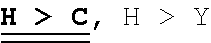 | |
| | | | | | | CGT | H > R | |
| CCA | P | CCG | = | TTA, TCA, | P > L, P > S | TTG, TCA, | P > L, P > S, = | |
| | | | | CTA | | CCG, CTA, | | |
| | | | | | | TTA, TCG, | | |
| | | | | | | CTG | | |
| CGA | R | CGG | = | TGA | R > STOP | 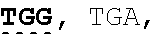 | 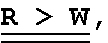 | |
| | | | | | | CGG | R > STOP, = | |
| CTA | L | CTG | = | TTA | = | TTG, TTA, | = | |
| | | | | | | CTG | | |
| GAC | D | GGC | D > G | GAT | = | GGT, GAT, | D > G, = | |
| | | | | | | GGC | | |
| GCA | A | GCG | = | GTA | A > V | GTG, GTA, | A > V, = | |
| | | | | | | GCG | | |
| TAC | Y | TGC | Y > C | TAT | = | TGT, TAT, | Y > C, = | |
| | | | | | | TGC | | |
| TCA | S | TCG | = | TTA | S > L | TTG, TTA, | S > L, = | |
| | | | | | | TCG | | |
| AGT | S | AGC | = | AAT | S > N | AAC, AAT, | S > N, = | T > C |
| | | | | | | AGC | | and |
| ATG | M | ACG | M > T | ATA | M > I | ACA, ATA, | M > T, M > I | G > A |
| | | | | | | ACG | | |
| CGT | R | CGC | = | CAT | R > H | CAC, CAT, | R > H, = | |
| | | | | | | CGC | | |
| CTG | L | CCG | L > P | CTA | = | CCA, CTA, | L > P, = | |
| | | | | | | CCG | | |
| GAT | D | GAC | = | AAT | D > N | AAC, AAT, | D > N, = | |
| | | | | | | GAC | | |
| GCT | A | GCC | = | ACT | A > T | ACC, ACT, | A > T, = | |
| | | | | | | GCC | | |
| GGT | G | GGC | = | AAT, AGT, | G > N, | AAC, AGT, | G > N, G > S, | |
| | | | | GAT | G > S, G > D | GGC, GAT, | =, G > D | |
| | | | | | | AAT, AGC, | | |
| | | | | | | GAC | | |
| GTA | V | GCA | V > A | ATA | V > I | 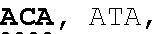 | 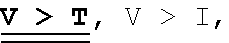 | |
| | | | | | | GCA | V > A | |
| GTC | V | GCC | V > A | ATC | V > I | 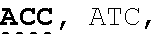 | 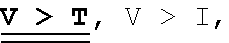 | |
| | | | | | | GCC | V > A | |
| GTG | V | GCG | V > A | ATA, ATG, | V > I, V > M, | 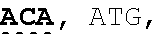 | 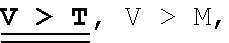 | |
| | | | | GTA | = | GCG, GTA, | V > A, =, V > I | |
| | | | | | | 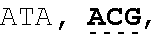 | | |
| | | | | | | GCA | | |
| GTT | V | GCC, GCT, | V > A, = | ATT | V > I | 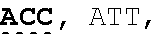 | 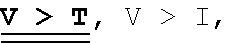 | |
| | | GTC | | | | GCT, GTC | V > A, = | |
| | | | | | | 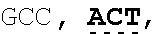 | | |
| | | | | | | ATC | | |
| TAG | STOP | CAG | STOP > Q | TAA | = | CAA, TAA, | STOP > Q, = | |
| | | | | | | CAG | | |
| TCG | S | CCG | S > P | TCA | = | CCA, TCA, | S > P, = | |
| | | | | | | CCG | | |
| TGA | STOP | CGA | STOP > R | TAA | = | 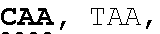 | 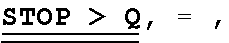 | |
| | | | | | | CGA | STOP > R | |
| TGC | C | CGC | C > R | TAC | C > Y | 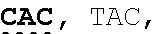 | 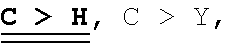 | |
| | | | | | | CGC | C > R | |
| TGG | W | CGG | W > R | TAA, TAG, | W > STOP | 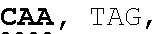 | 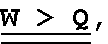 | |
| | | | | TGA | | CGG, TGA, | W > STOP, | |
| | | | | | | 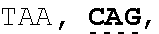 | W > R | |
| | | | | | | CGA | | |
| TGT | C | CGC, CGT, | C > R, = | TAT | C > Y | 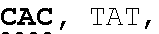 | 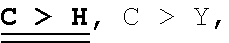 | |
| | | TGC | | | | CGT, TGC, | C > R, = | |
| | | | | | | 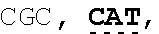 | | |
| | | | | | | TAC | | |
| TTG | L | CCG, CTG, | L > P, = | TTA | = | CCA, TTA, | L > P, = , L > S | |
| | | TCG | L > S | | | CTG, TCG, | | |
| | | | | | | CCG, CTA, | | |
| | | | | | | TCA | | |

WT = wild type;
AA = amino acid.
Bolded = unique codon mutation by SPACE with respect to WT codon.
Bolded and dash-underlined = same as bolded, but also resulting in unique amino acid change with respect to WT codon.
Double underlined = unique amino acid change by SPACE with respect to WT codon

TABLE E
Specific targetable mutations from the ClinVar database that can
be corrected with SPACE using Cas9 proteins with NGG, NGA, NG and AA
PAM recognition.

| | | Reference | Reference | | | |
|---|---|---|---|---|---|---|
| | | proto- | proto- | Reference | | |
| | | spacer and | spacer | proto- | | |
| | | NG[GA]. | and | spacer and | Amino | |
| | Gene | PAM | NG.PAM | NAA.PAM | acid | Associated genetic |
| ClinvarID | type | sequence | sequence | sequence | chage | disease |
| 12750 | TG > | TGGTTCT | TGGTTC | | Q/W | Immunodeficiency |
| | CA | GGCCCT | TGGCCC | | | due to defect in cd3- |
| | | GCTGG | TGCTG | | | zeta |
| 17145 | TG > | | TGTCCC | | Q/W | Intervertebral disc |
| | CA | | GCCTGT | | | disease |
| | | | CCTG | | | |
| 128121 | TG > | | TGAATA | | Q/* | Pre-B-cell acute |
| | CA | | AATAAT | | | lymphoblastic |
| | | | TTTTCG | | | leukemia\|Familial |
| | | | | | | cancer of |
| | | | | | | breast\|Hereditary |
| | | | | | | cancer-predisposing |
| | | | | | | syndrom |
| 128424 | TG > | | | TGTCATT | DI/DV | Familial |
| | CA | | | TATTCTT | | hypercholesterolemias |
| | | | | TCAA | | \|Familial |
| | | | | | | hypercholesteroiemia |
| | | | | | | \|Hypercholesterolemia, |
| | | | | | | autosomal |
| | | | | | | dominant, type |
| | | | | | | B\|Familial |
| | | | | | | hypobetalipoproteinemia |
| | | | | | | \|Hypobetalipoproteinemia, |
| | | | | | | familial, 1 |
| 188253 | TG > | TGTTTCA | TGTTTC | | VV/VI | Hereditary breast |
| | CA | ATGTTTA | AATGTT | | | and ovarian cancer |
| | | AGA | TAAG | | | syndrome not |
| | | | | | | provided |
| 199700 | GGT > | GGTCCA | GGTCCA | | G/S | Thoracic aortic |
| | AGC | GGTGCT | GGTGCT | | | aneurysm and aortic |
| | | GATGG | GATG | | | dissection |
| 220300 | TG > | TGGCGT | TGGCGT | | AG/AS | Hereditary cancer- |
| | CA | CTGTAG | CTGTAG | | | predisposing |
| | | GAAGG | GAAG | | | syndrome Hereditary |
| | | | | | | diffuse gastric |
| | | | | | | cancer |
| 239762 | TG > | | TGCACT | | — | Baller-Gerold |
| | CA | | CACTCT | | | syndrome |
| | | | GGCCT | | | |
| | | | G | | | |
| 240354 | TG > | TGTTTAT | TGTTTA | | NV/NI | Gastrointestinal |
| | CA | GCTTTAA | TGCTTT | | | stroma tumor |
| | | AAGG | AAAAG | | | |
| 240644 | TG > | TGTACCT | TGTACC | | — | Primary dilated |
| | CA | GACTTT | TGACTT | | | cardiomyopathy |
| | | GGAGG | TGGAG | | | |
| 242209 | TG > | TGCCAT | TGCCAT | | A/V | Brugada |
| | CA | GCGCTT | GCGCTT | | | syndrome\|not |
| | | CTCGA | CTCG | | | specified\|Cardiovascular |
| | | | | | | phenotype |
| 251438 | GT > | GTGTGA | GTGTGA | | QC/QR | Familial |
| | AC | CCGGGA | CCGGG | | | hypercholesterolemia |
| | | ATATGA | AATATG | | | |
| 300363 | TG > | | TGTCTT | | TV/TI | Histiocytosis- |
| | CA | | CCTCGT | | | lymphadenopathy |
| | | | GCTCTG | | | plus syndrome |
| 313564 | TG > | TGGGAT | TGGGAT | TGGGAT | W/Q | Emery-Dreifuss |
| | CA | GAAGAA | GAAGAA | GAAGAA | | muscular |
| | | ATAGA | ATAG | ATAGAA | | dystrophy\|Emery- |
| | | | | | | Dreifuss muscular |
| | | | | | | dystrophy 5, |
| | | | | | | autosomal dominant |
| 371798 | TGT > | TGTCGC | TGTCGC | | PV/PT | Mitochondrial |
| | CAC | GAAGGA | GAAGGA | | | complex II |
| | | CCTGG | CCTG | | | deficiency\|Paragang |
| | | | | | | liomas 5 |
| 372212 | TGC > | | TGCCTC | | LP/PS | 3-methylglutaconic |
| | CAT | | TGGGAC | | | aciduria, type VIII |
| | | | GCTCAG | | | |
| 404057 | TG > | TGTGAT | TGTGAT | | C/H | Werner syndrome |
| | CA | GTCAAC | GTCAAC | | | |
| | | AAAAGG | AAAAG | | | |
| 405615 | TG > | | TGAGAG | | S/L | Colorectal cancer, |
| | CA | | CTCAGT | | | susceptibility to, 12 |
| | | | GGAG | | | |
| 406478 | TG > | TGGACA | TGGACA | TGGACA | HG/HR | Long QT syndrome |
| | CA | CACCGT | CACCGT | CACCGT | | |
| | | GGTAAA | GGTAAA | GGTAA | | |
| | | GA | G | | | |
| 410997 | TG > | TGGGAT | TGGGAT | | M/T | Ciliary dyskinesia, |
| | CA | GCCTTG | GCCTTG | | | primary, 28 |
| | | CTTCTGA | CTTCTG | | | |
| 412369 | TG > | TGTCGC | TGTCGC | | PV/PI | Mitochondrial |
| | CA | GAAGGA | GAAGGA | | | complex II |
| | | CCTGG | CCTG | | | deficiency\|Paragang |
| | | | | | | liomas 5 |
| 456874 | TG > | TGCTGC | TGCTGC | | SS/SG | Long QT syndrome |
| | CA | GGGGGG | GGGGG | | | |
| | | ACCTGC | GACCTG | | | |
| | | GG | | | | |
| 464434 | GCCT > | | GCCTG | GCCTGGC | QA/RL | Arrhythmogenic |
| | AGGC | | GCCGA | CGACAGT | | right ventricular |
| | | | CAGTCA | CAA | | cardiomyopathy, |
| | | | AG | | | type 9\|not specified |
| 469582 | TG > | TGGAGG | TGGAG | TGGAGG | GG/GR | Atrial fibrillation, |
| | CA | AGGCGG | GAGGC | AGGCGG | | familial, 7 |
| | | CCAGA | GGCCA | CCAGAA | | |
| | | G | | | | |
| 470429 | TG > | | | TGACAG | DD/DN | Rhabdoid tumor |
| | CA | | | TGAGGA | | predisposition |
| | | | | GGAACA | | syndrome 2 |
| | | | | A | | |
| 472109 | TG > | | TGCGCC | | TA/TT | Myofibrillar |
| | CA | | TGGAGA | | | myopathy, filamin C- |
| | | | CTACAG | | | related\|Myopathy, |
| | | | | | | distal, |
| | | | | | | 4\|Cardiomyopathy, |
| | | | | | | familial hypertrophic, |
| | | | | | | 26\|Dilated |
| | | | | | | Cardiomyopathy, |
| | | | | | | Dominant |
| 476408 | TG > | TGCCTTT | TGCCTT | | GA/GT | Bethlem myopathy 1 |
| | CA | GGACTG | TGGACT | | | |
| | | AAAGG | GAAAG | | | |
| 480118 | GT > | GTACCA | GTACCA | | LY/LH | Hereditary cancer- |
| | AC | GATCCC | GATCCC | | | predisposing |
| | | ACAGA | ACAG | | | syndrome |
| 485859 | GAT > | GATGAA | GATGAA | GATGAA | D/N | Hereditary cancer- |
| | AAC | GCCTTA | GCCTTA | GCCTTA | | predisposing |
| | | AATAAAG | AATAAA | AATAA | | syndrome |
| | | A | G | | | |
| 491715 | GTT > | GTTTATC | GTTTAT | | — | Hereditary cancer- |
| | ATC | AGCAAG | CAGCAA | | | predisposing |
| | | GAGA | GGAG | | | syndrome not |
| | | | | | | specified |
| 529164 | TG > | | TGGCAA | | HG/HS | Netherton syndrome |
| | CA | | CACCTG | | | |
| | | | CTCCAT | | | |
| | | | G | | | |
| 533984 | TG > | TGTGTC | TGTGTC | | T/M | Nemaline myopathy |
| | CA | GTGGAT | GTGGAT | | | 2 |
| | | TGTGG | TGTG | | | |
| 534930 | TG > | TGAGAG | TGAGAG | | LE/LK | Spinal muscular |
| | CA | AGACGC | AGACGC | | | atrophy, distal, |
| | | GGAGG | GGAG | | | autosomal |
| | | | | | | recessive, |
| | | | | | | 1\|Charcot-Marie- |
| | | | | | | Tooth disease, |
| | | | | | | axonal, type 2S |
| 535289 | GT > | GTTGGA | GTTGGA | GTTGGA | T/V | Limb-girdle |
| | AC | CGACCT | CGACCT | CGACCT | | muscular dystrophy, |
| | | TTGAATG | TTGAAT | TTGAA | | type 2J\|Dilated |
| | | G | G | | | cardiomyopathy 1G |
| 536518 | TG > | | TGAGGC | | TE/TK | Limb-girdle |
| | CA | | | TGGGGG | | muscular dystrophy, |
| | | | | TGGAA | | type 2A |
| 541194 | GT > | GTCCTA | GTCCTA | | - | Myasthenic |
| | AC | GACCAG | GACCAG | | | syndrome, |
| | | TGCCCG | TGCCCG | | | congenital, 8 |
| | | G | | | | |
| 566099 | TG > | TGGGAA | TGGGAA | | AG/AR | Colorectal cancer 10 |
| | CA | ATACGC | ATACGC | | | |
| | | CCTGA | CCTG | | | |
| 571908 | TG > | TGCTTA | TGCTTA | | SI/SV | Nemaline myopathy |
| | CA | GTGCAC | GTGCAC | | | 2 |
| | | TGGCAG | TGGCAG | | | |
| | | A | | | | |
| 576182 | TCG > | | TCGGCT | | R/W | Rigidity and |
| | CCA | | CGCCG | | | multifocal seizure |
| | | | CCTGCA | | | syndrome, lethal |
| | | | G | | | neonatal |
| 576750 | TG > | TGCCCT | TGCCCT | | A/V | Colorectal cancer, |
| | CA | GCGCTG | GCGCT | | | susceptibility to, 12 |
| | | TGAGG | GTGAG | | | |
| 627700 | TG > | TGGATTT | TGGATT | | P/L | Hereditary cancer- |
| | CA | CTGGAA | TCTGGA | | | predisposing |
| | | GTAGA | AGTAG | | | syndrome |
| 628274 | GGT > | GGTTCTT | GGTTCT | | EP/EL | Hereditary cancer- |
| | AGC | GGTCCT | TGGTCC | | | predisposing |
| | | CAGG | TCAG | | | syndrome |
| 630535 | GT > | | | GTGTCT | T/V | Hereditary cancer- |
| | AC | | | GGGATG | | predisposing |
| | | | | CTGAA | | syndrome |
| 647607 | GT > | | GTGCCT | | R/H | Ehlers-Danlos |
| | AC | | GCCCC | | | syndrome progeroid |
| | | | CCAGAG | | | type |
| 647708 | TG > | | TGGCAA | | SG/SS | Long QT syndrome |
| | CA | | GTGCAA | | | |
| | | | CGTG | | | |
| 652124 | TG > | TGTGGA | TGTGGA | | C/H | Myofibrillar |
| | CA | CAGGTG | CAGGTG | | | myopathy, BAG3- |
| | | GCAGCG | GCAG | | | related Dilated |
| | | G | | | | cardiomyopathy |
| | | | | | | 1HH |
| 653067 | TG > | TGCTCC | TGCTCC | | DA/DT | Hereditary |
| | CA | TGTGCT | TGTGCT | | | pancreatitis |
| | | GAGCCA | GAGCCA | | | |
| | | GG | G | | | |
| 657209 | TG > | TGTTGCT | TGTTGC | | RV/RI | Neuropathy, |
| | CA | ATTGAAC | TATTGA | | | hereditary sensory, |
| | | ACCTGG | ACACCT | | | with spastic |
| | | | G | | | paraplegia, |
| | | | | | | autosomal recessive |
| 660313 | TG > | | TGCAGA | | GA/GT | Chromosome 9q |
| | CA | | GCAGG | | | deletion syndrome |
| | | | CGGCC | | | |
| | | | G | | | |
| 662382 | TG > | TGTTCTG | TGTTCT | | AV/AI | Renal hamartomas |
| | CA | GTCAAG | GGTCAA | | | nephroblasto- |
| | | GTGA | GGTG | | | matosis and fetal |
| | | | | | | gigantism |

TABLE F
Specific targetable MNV mutations from the gnomAD database that can
be modelled with SPACE using Cas9 proteins with NGG PAM recognition.

| Gene_name | MNV | MNV_codons | MNV_AAs | MNV_category |
|---|---|---|---|---|
| C1orf170 | 1-911594-CA-TG | gTG/gCA | V/A | Unchanged |
| C1orf170 | 1-915225-CAA-TAG | TtG/CtA | L | Unchanged |
| ARHGEF16 | 1-3383896-CCA-TCG | CcA/TcG | P/S | Unchanged |
| ARHGEF16 | 1-3383897-CA-TG | cCA/cTG | P/L | Unchanged |
| CHD5 | 1-6184090-CGA-TGG | TcG/CcA | S/P | Unchanged |
| SLC45A1 | 1-8390494-CA-TG | cCA/cTG | P/L | Unchanged |
| H6PD | 1-9324669-CA-TG | tCA/tTG | S/L | Unchanged |
| PEX14 | 1-10689923-AC-GT | gAC/gGT | D/G | Unchanged |
| PRAMEF1 | 1-12855598-AC-TG | aAC/aTG | N/M | Changed missense |
| PRAMEF2 | 1-12921087-AC-TG | aAC/aTG | N/M | Changed missense |
| PRAMEF4 | 1-12942989-CA-TG | TGt/CAt | C/H | Changed missense |
| PRDM2 | 1-14143002-CA-TG | cCA/cTG | P/L | Unchanged |
| ARHGEF10L | 1-18023364-AC-GT | aAC/aGT | N/S | Unchanged |
| UBR4 | 1-19433449-AAC-GAT | GtT/AtC | V/I | Unchanged |
| EMC1 | 1-19566381-CA-TG | tTG/tCA | L/S | Unchanged |
| AKR7A3 | 1-19612476-AC-GT | GTg/ACg | V/T | Changed missense |
| STPG1 | 1-24710404-CA-TG | aTG/aCA | M/T | Partially changed missense |
| GRIK3 | 1-37267555-AC-GT | GTc/ACc | V/T | Changed missense |
| NFYC | 1-41204567-AGC-GGT | AgC/GgT | S/G | Unchanged |
| HIVEP3 | 1-42046527-CA-GT | aTG/aAC | M/N | Changed missense |
| GUCA2B | 1-42619138-CA-TG | gCA/gTG | A/V | Unchanged |
| FOXJ3 | 1-42693596-CA-TG | gTG/gCA | V/A | Unchanged |
| AKR1A1 | 1-46027527-AC-GT | ACc/GTc | T/V | Changed missense |
| MAST2 | 1-46500882-AC-GT | aAC/aGT | N/S | Unchanged |
| SLC1A7 | 1-53558270-AC-GT | cGT/cAC | R/H | Unchanged |
| MROH7 | 1-55119143-CA-TG | CAt/TGt | H/C | Changed missense |
| MROH7-TTC4 | 1-55119143-CA-TG | CAt/TGt | H/C | Changed missense |
| MROH7-TTC4 | 1-55139740-CA-TG | CAc/TGc | H/C | Changed missense |
| MROH7 | 1-55139740-CA-TG | CAc/TGc | H/C | Changed missense |
| KANK4 | 1-62728783-CA-TG | gTG/gCA | V/A | Unchanged |
| KANK4 | 1-62728784-AC-GT | GTg/ACg | V/T | Changed missense |
| ATP5F1 | 1-112002206-AC-GT | cAC/cGT | H/R | Unchanged |
| PPM1J | 1-113255388-AC-GT | GTt/ACt | V/T | Changed missense |
| PTPN22 | 1-114377567-CA-TG | TGg/CAg | W/Q | Rescued nonsense |
| NBPF16 | 1-148756446-AC-GT | aAC/aGT | N/S | Unchanged |
| SNX27 | 1-151634729-AC-GT | ACa/GTa | T/V | Changed missense |
| HRNR | 1-152190349-CA-TG | tTG/tCA | L/S | Unchanged |
| FLG | 1-152283333-CA-TG | gTG/gCA | V/A | Unchanged |
| FLG | 1-152286385-CA-TG | TGg/CAg | W/Q | Rescued nonsense |
| LCE1E | 1-152760074-CA-TG | tCA/tTG | S/L | Unchanged |
| LOR | 1-153233510-AGC-GGT | AgC/GgT | S/G | Unchanged |
| HCN3 | 1-155247394-CA-GT | CAg/GTg | Q/V | Changed missense |
| GON4L | 1-155735584-AC-TG | GTg/CAg | V/Q | Changed missense |
| GON4L | 1-155736381-CA-TG | tTG/tCA | L/S | Unchanged |
| FCRL5 | 1-157509024-AC-GT | gGT/gAC | G/D | Unchanged |
| ATP1A4 | 1-160141195-AC-GT | tAC/tGT | Y/C | Unchanged |
| POGK | 1-166818565-CA-TG | gCA/gTG | A/V | Unchanged |
| ADCY10 | 1-167849414-ACC-GCT | GgT/AgC | G/S | Unchanged |
| XCL1 | 1-168550337-CA-TG | aCA/aTG | T/M | Changed missense |
| KIAA1614 | 1-180913597-AAC-GAT | AaC/GaT | N/D | Unchanged |
| SMG7 | 1-183510142-AC-GT | cAC/cGT | H/R | Unchanged |
| FAM129A | 1-184765071-CA-TG | cTG/cCA | L/P | Unchanged |
| CFHR1 | 1-196797356-CA-TG | aCA/aTG | T/M | Changed missense |
| DSTYK | 1-205130412-CA-TG | TGt/CAt | C/H | Changed missense |
| CR1 | 1-207753620-CA-TG | CAt/TGt | H/C | Changed missense |
| CAMK1G | 1-209782342-CA-TG | cCA/cTG | P/L | Unchanged |
| TRAF5 | 1-211533351-CA-TG | gCA/gTG | A/V | Unchanged |
| FAM71A | 1-212799870-AAC-GAT | AaC/GaT | N/D | Unchanged |
| HHIPL2 | 1-222721288-CA-TG | tTG/tCA | L/S | Unchanged |
| OBSCN | 1-228402120-CA-TG | CAg/TGg | Q/W | Rescued nonsense |
| OBSCN | 1-228433216-CA-TG | aCA/aTG | T/M | Changed missense |
| OBSCN | 1-228465346-AAC-GAT | AaC/GaT | N/D | Unchanged |
| OBSCN | 1-228503676-CA-TG | CAc/TGc | H/C | Changed missense |
| OBSCN | 1-228503677-AC-GT | cAC/cGT | H/R | Unchanged |
| OBSCN | 1-228560138-CA-TG | tCA/tTG | S/L | Unchanged |
| HEATR1 | 1-236716959-CGA-TGG | TcG/CcA | S/P | Unchanged |
| MTR | 1-237054568-CA-TG | gCA/gTG | A/V | Unchanged |
| OR2L8 | 1-248112835-CA-TG | CAc/TGc | H/C | Changed missense |
| OR2T33 | 1-248436616-AC-GT | tGT/tAC | C/Y | Unchanged |
| OR2T3 | 1-248637064-CA-TG | cCA/cTG | P/L | Unchanged |
| OR2T11 | 1-248790074-CA-TG | TGc/CAc | C/H | Changed missense |
| AKR1C1 | 10-5010571-CA-TG | aCA/aTG | T/M | Changed missense |
| ECHDC3 | 10-11805353-CA-TG | gCA/gTG | A/V | Unchanged |
| ITGA8 | 10-15573050-AC-GT | GTa/ACa | V/T | Changed missense |
| PTER | 10-16547056-CAA-TAG | CaA/TaG | Q/* | Unchanged |
| CUBN | 10-16918972-AGC-GGT | GcT/AcC | A/T | Unchanged |
| EBLN1 | 10-22498208-CA-TG | aTG/aCA | M/T | Partially changed missense |
| KIAA1217 | 10-24762464-CA-TG | gCA/gTG | A/V | Unchanged |
| UNC5B | 10-73056495-CA-TG | gCA/gTG | A/V | Unchanged |
| RPS24 | 10-79814598-CA-TG | CAc/TGc | H/C | Changed missense |
| SFTPD | 10-81706323-CA-TG | aTG/aCA | M/T | Partially changed missense |
| CDHR1 | 10-85960394-CA-TG | gCA/gTG | A/V | Unchanged |
| CEP55 | 10-95262981-AC-GT | ACa/GTa | T/V | Changed missense |
| PYROXD2 | 10-100148175-CA-TG | aTG/aCA | M/T | Partially changed missense |
| HPS1 | 10-100202987-AC-GT | GTc/ACc | V/T | Changed missense |
| NRAP | 10-115411613-CA-TG | gTG/gCA | V/A | Unchanged |
| NRAP | 10-115411614-AC-GT | GTg/ACg | V/T | Changed missense |
| DMBT1 | 10-124358455-CA-TG | tCA/tTG | S/L | Unchanged |
| AL450307.1 | 10-133608209-CA-TG | TGc/CAc | C/H | Changed missense |
| STK32C | 10-134036193-AC-GT | cGT/cAC | R/H | Unchanged |
| SPRN | 10-135237060-AC-GT | gGT/gAC | G/D | Unchanged |
| CYP2E1 | 10-135342049-AC-TG | cAC/cTG | H/L | Partially changed missense |
| PSMD13 | 11-244166-AC-GT | aAC/aGT | N/S | Unchanged |
| ATHL1 | 11-290815-CA-TG | gCA/gTG | A/V | Unchanged |
| ANO9 | 11-428384-CA-TG | TGc/CAc | C/H | Changed missense |
| MUC2 | 11-1075678-AC-GT | aAC/aGT | N/S | Unchanged |
| MUC5B | 11-1264361-CA-TG | aCA/aTG | T/M | Changed missense |
| MOB2 | 11-1507730-CA-TG | cTG/cCA | L/P | Unchanged |
| OR51B2 | 11-5345169-CA-TG | TGt/CAt | C/H | Changed missense |
| OR51B5 | 11-5364692-AGC-GGT | GcT/AcC | A/T | Unchanged |
| OR52D1 | 11-5510283-CA-TG | tCA/tTG | S/L | Unchanged |
| OR52H1 | 11-5566029-CA-TG | TGc/CAc | C/H | Changed missense |
| OR52L1 | 11-6007271-CA-TG | TGg/CAg | W/Q | Rescued nonsense |
| OR2AG2 | 11-6789256-CA-TG | cTG/cCA | L/P | Unchanged |
| OR2AG1 | 11-6807200-CA-TG | cCA/cTG | P/L | Unchanged |
| ZNF214 | 11-7022159-CA-TG | TGt/CAt | C/H | Changed missense |
| SWAP70 | 11-9754220-AC-GT | aAC/aGT | N/S | Unchanged |
| MRGPRX3 | 11-18159119-ATC-GTT | AtC/GtT | I/V | Unchanged |
| MRGPRX4 | 11-18195722-CA-TG | CAg/TGg | Q/W | Rescued nonsense |
| SAA2 | 11-18267026-AC-GT | cGT/cAC | R/H | Unchanged |
| RAG1 | 11-36597733-CA-TG | gCA/gTG | A/V | Unchanged |
| C11orf96 | 11-43946967-AC-GT | ACg/GTg | T/V | Changed missense |
| DGKZ | 11-46387867-CA-TG | CAg/TGg | Q/W | Rescued nonsense |
| DGKZ | 11-46397675-CA-GT | CAc/GTc | H/V | Changed missense |
| TRIM64C | 11-49080503-CA-TG | tTG/tCA | L/S | Unchanged |
| OR4C16 | 11-55339829-AC-GT | ACc/GTc | T/V | Changed missense |
| OR4C16 | 11-55339829-ACC-GCT | AcC/GcT | T/A | Unchanged |
| OR5L1 | 11-55579340-CA-TG | aCA/aTG | T/M | Changed missense |
| OR5R1 | 11-56185344-CA-TG | TGc/CAc | C/H | Changed missense |
| OR4D10 | 11-59245494-CTA-TTG | CtA/TtG | L | Unchanged |
| MS4A4A | 11-60059738-CA-TG | CAg/TGg | Q/W | Rescued nonsense |
| CD5 | 11-60892605-CA-TG | CAc/TGc | H/C | Changed missense |
| NUDT22 | 11-63997328-CA-TG | CAg/TGg | Q/W | Rescued nonsense |
| SLC22A12 | 11-64367324-CA-TG | gCA/gTG | A/V | Unchanged |
| PCNXL3 | 11-65385605-CA-TG | CAg/TGg | Q/W | Rescued nonsense |
| SPTBN2 | 11-66468468-CA-TG | gTG/gCA | V/A | Unchanged |
| TBC1D10C | 11-67176897-CA-TG | tCA/tTG | S/L | Unchanged |
| GPR152 | 11-67218909-AGC-GGT | GcT/AcC | A/T | Unchanged |
| AIP | 11-67258390-CA-TG | CAg/TGg | Q/W | Rescued nonsense |
| ALDH3B2 | 11-67434160-CA-TG | aTG/aCA | M/T | Partially changed missense |
| ANO1 | 11-70007391-CA-TG | gCA/gTG | A/V | Unchanged |
| DEFB108B | 11-71548543-CA-TG | CAa/TGa | Q/* | Unchanged |
| KDM4E | 11-94759058-CA-TG | CAg/TGg | Q/W | Rescued nonsense |
| EXPH5 | 11-108383675-CA-TG | cTG/cCA | L/P | Unchanged |
| ZC3H12C | 11-110035260-CGA-TGG | CgA/TgG | R/W | Rescued nonsense |
| LAYN | 11-111420363-CA-TG | cCA/cTG | P/L | Unchanged |
| BUD13 | 11-116627892-CA-TG | TGg/CAg | W/Q | Rescued nonsense |
| BACE1 | 11-117160346-CA-TG | TGc/CAc | C/H | Changed missense |
| CEP164 | 11-117267883-CA-TG | CAg/TGg | Q/W | Rescued nonsense |
| AMICA1 | 11-118074337-AC-GT | GTg/ACg | V/T | Changed missense |
| TMEM25 | 11-118405067-CA-TG | CAa/TGa | Q/* | Unchanged |
| BCL9L | 11-118771746-CA-TG | gTG/gCA | V/A | Unchanged |
| VPS11 | 11-118947633-AC-GT | tAC/tGT | Y/C | Unchanged |
| TECTA | 11-120996291-CA-TG | gCA/gTG | A/V | Unchanged |
| UBASH3B | 11-122665436-CA-TG | tCA/tTG | S/L | Unchanged |
| OR6T1 | 11-123814489-AC-GT | aGT/aAC | S/N | Unchanged |
| OR10G4 | 11-123886319-CA-TG | aCA/aTG | T/M | Changed missense |
| ST14 | 11-130066334-AC-GT | aAC/aGT | N/S | Unchanged |
| TULP3 | 12-3046801-CA-TG | aCA/aTG | T/M | Changed missense |
| ANO2 | 12-6030404-CA-TG | gTG/gCA | V/A | Unchanged |
| CD163L1 | 12-7527123-CA-TG | aTG/aCA | M/T | Partially changed missense |
| SMCO2 | 12-27648785-CA-TG | gCA/gTG | A/V | Unchanged |
| CCDC65 | 12-49308283-CA-TG | CAc/TGc | H/C | Changed missense |
| CERS5 | 12-50537814-CA-TG | TGt/CAt | C/H | Changed missense |
| ANKRD33 | 12-52284499-CA-TG | CAg/TGg | Q/W | Rescued nonsense |
| OR10P1 | 12-56031610-CA-TG | tCA/tTG | S/L | Unchanged |
| ARHGEF25 | 12-58010162-CA-TG | CAg/TGg | Q/W | Rescued nonsense |
| PTPRB | 12-70928724-ATC-GTT | GaT/AaC | D/N | Unchanged |
| TRHDE | 12-73014916-CA-TG | aCA/aTG | T/M | Changed missense |
| MYO1H | 12-109853348-CA-TG | cCA/cTG | P/L | Unchanged |
| HVCN1 | 12-111099002-CA-TG | aTG/aCA | M/T | Partially changed missense |
| DTX1 | 12-113532615-CGA-TGG | CgA/TgG | R/W | Rescued nonsense |
| GTF2H3 | 12-124144394-CA-TG | cCA/cTG | P/L | Unchanged |
| DNAH10 | 12-124387585-CA-TG | gCA/gTG | A/V | Unchanged |
| SCARB1 | 12-125284748-AGC-GGT | GcT/AcC | A/T | Unchanged |
| EP400 | 12-132528770-AC-GT | ACc/GTc | T/V | Changed missense |
| EP400 | 12-132528770-ACC-GCT | AcC/GcT | T/A | Unchanged |
| TNFRSF19 | 13-24234516-CA-TG | tCA/tTG | S/L | Unchanged |
| C1QTNF9B | 13-24465716-CA-TG | gTG/gCA | V/A | Unchanged |
| C1QTNF9B | 13-24468328-CA-TG | TGt/CAt | C/H | Changed missense |
| ATP12A | 13-25265102-CA-TG | gCA/gTG | A/V | Unchanged |
| RNF17 | 13-25419175-CA-TG | aCA/aTG | T/M | Changed missense |
| SLC46A3 | 13-29287551-AC-GT | GTt/ACt | V/T | Changed missense |
| FRY | 13-32676113-CA-TG | aCA/aTG | T/M | Changed missense |
| TMTC4 | 13-101287339-AC-GT | GTt/ACt | V/T | Changed missense |
| PROZ | 13-113818899-CA-TG | aCA/aTG | T/M | Changed missense |
| CDC16 | 13-115016035-CA-TG | aCA/aTG | T/M | Changed missense |
| TRAV27 | 14-22616546-CA-TG | gCA/gTG | A/V | Unchanged |
| TRAV36DV7 | 14-22695128-ATC-GTT | AtC/GtT | I/V | Unchanged |
| AE000662.92 | 14-23026575-AAC-GAT | AaC/GaT | N/D | Unchanged |
| SLC8A3 | 14-70634087-AC-GT | cGT/cAC | R/H | Unchanged |
| ADAM21 | 14-70924566-CA-GT | gCA/gGT | A/G | Unchanged |
| TMEM63C | 14-77703056-CA-TG | gCA/gTG | A/V | Unchanged |
| BDKRB2 | 14-96707456-CA-TG | aCA/aTG | T/M | Changed missense |
| KIF26A | 14-104643408-CA-TG | gCA/gTG | A/V | Unchanged |
| KIF26A | 14-104643857-CGA-TGG | CgA/TgG | R/W | Rescued nonsense |
| AHNAK2 | 14-105410775-ATC-GTT | GaT/AaC | D/N | Unchanged |
| AHNAK2 | 14-105411162-CA-TG | cTG/cCA | L/P | Unchanged |
| AHNAK2 | 14-105414474-CA-TG | aTG/aCA | M/T | Partially changed missense |
| AHNAK2 | 14-105414979-AC-GT | GTg/ACg | V/T | Changed missense |
| AHNAK2 | 14-105415122-CA-TG | cTG/cCA | L/P | Unchanged |
| AHNAK2 | 14-105415473-CA-TG | gTG/gCA | V/A | Unchanged |
| AHNAK2 | 14-105415515-CA-TG | gTG/gCA | V/A | Unchanged |
| AHNAK2 | 14-105415516-AC-GT | GTg/ACg | V/T | Changed missense |
| AHNAK2 | 14-105416454-CA-TG | aTG/aCA | M/T | Partially changed missense |
| AHNAK2 | 14-105416505-CA-TG | aTG/aCA | M/T | Partially changed missense |
| AHNAK2 | 14-105416958-CA-TG | gTG/gCA | V/A | Unchanged |
| AHNAK2 | 14-105416959-AC-GT | GTg/ACg | V/T | Changed missense |
| AHNAK2 | 14-105417957-ACC-GCT | GgT/AgC | G/S | Unchanged |
| AHNAK2 | 14-105419736-CA-TG | tTG/tCA | L/S | Unchanged |
| IGHV1-3 | 14-106471267-CA-TG | aTG/aCA | M/T | Partially changed missense |
| IGHV3-9 | 14-106552480-CA-TG | aTG/aCA | M/T | Partially changed missense |
| IGHV3-53 | 14-107049101-CA-TG | TGg/CAg | W/Q | Rescued nonsense |
| IGHV4-61 | 14-107095378-CGA-TGG | TcG/CcA | S/P | Unchanged |
| GOLGA8T | 15-30434160-CA-TG | CAg/TGg | Q/W | Rescued nonsense |
| GOLGA8K | 15-32686364-CA-TG | cTG/cCA | L/P | Unchanged |
| THBS1 | 15-39880822-AC-GT | ACa/GTa | T/V | Changed missense |
| DUOX2 | 15-45388098-CA-TG | gTG/gCA | V/A | Unchanged |
| DUOX1 | 15-45444517-CA-TG | aCA/aTG | T/M | Changed missense |
| MYO5C | 15-52531939-CAA-TAG | TtG/CtA | L | Unchanged |
| PIF1 | 15-65113686-CA-TG | TGt/CAt | C/H | Changed missense |
| KIF23 | 15-69728947-CCA-TCG | CcA/TcG | P/S | Unchanged |
| KIF23 | 15-69728948-CA-TG | cCA/cTG | P/L | Unchanged |
| ZNF592 | 15-85345509-CA-TG | cCA/cTG | P/L | Unchanged |
| SLC28A1 | 15-85478408-CAA-TAG | CaA/TaG | Q/* | Unchanged |
| KIF7 | 15-90174789-CGA-TGG | TcG/CcA | S/P | Unchanged |
| UNC45A | 15-91489918-CA-TG | cCA/cTG | P/L | Unchanged |
| FAM169B | 15-98995079-CGA-TGG | TcG/CcA | S/P | Unchanged |
| ADAMTS17 | 15-100692844-CA-TG | aTG/aCA | M/T | Partially changed missense |
| LRRK1 | 15-101598312-ACC-GCT | AcC/GcT | T/A | Unchanged |
| CAPN15 | 16-603470-CA-TG | cCA/cTG | P/L | Unchanged |
| PIGQ | 16-624114-AC-GT | ACg/GTg | T/V | Changed missense |
| WDR90 | 16-711904-CA-TG | cCA/cTG | P/L | Unchanged |
| CACNA1H | 16-1261218-CA-TG | tCA/tTG | S/L | Unchanged |
| CCDC154 | 16-1488646-AC-GT | cGT/cAC | R/H | Unchanged |
| ZNF598 | 16-2049002-CA-GT | TGt/ACt | C/T | Changed missense |
| MEFV | 16-3297073-ATC-GTT | GaT/AaC | D/N | Unchanged |
| OR2C1 | 16-3405993-CA-TG | tCA/tTG | S/L | Unchanged |
| C16orf71 | 16-4796392-CA-TG | CAg/TGg | Q/W | Rescued nonsense |
| TNFRSF17 | 16-12060163-AC-GT | aAC/aGT | N/S | Unchanged |
| EEF2K | 16-22269866-CA-TG | CAa/TGa | Q/* | Unchanged |
| NPIPB6 | 16-28354498-CA-TG | gTG/gCA | V/A | Unchanged |
| APOBR | 16-28508446-CA-TG | gCA/gTG | A/V | Unchanged |
| CTB-134H23.2 | 16-29057391-CA-TG | CAg/TGg | Q/W | Rescued nonsense |
| QPRT | 16-29708350-AC-GT | ACt/GTt | T/V | Changed missense |
| TAOK2 | 16-29998199-CA-TG | aCA/aTG | T/M | Changed missense |
| CNGB1 | 16-57957215-CA-TG | gTG/gCA | V/A | Unchanged |
| NDRG4 | 16-58545425-CA-TG | tCA/tTG | S/L | Unchanged |
| BCAR1 | 16-75269267-AGC-GGT | GcT/AcC | A/T | Unchanged |
| CNTNAP4 | 16-76532582-CA-TG | CAg/TGg | Q/W | Rescued nonsense |
| OSGIN1 | 16-83999032-CA-TG | tCA/tTG | S/L | Unchanged |
| ZNF469 | 16-88505634-CA-TG | cCA/cTG | P/L | Unchanged |
| RP11-368I7.4 | 16-89751860-CA-TG | TGc/CAc | C/H | Changed missense |
| MC1R | 16-89986607-CA-TG | aCA/aTG | T/M | Changed missense |
| TUBB3 | 16-89986607-CA-TG | aCA/aTG | T/M | Changed missense |
| DEF8 | 16-90020745-CA-GT | CAg/GTg | Q/V | Changed missense |
| GAS8 | 16-90109710-CA-TG | aCA/aTG | T/M | Changed missense |
| MYO1C | 17-1384066-AC-GT | gGT/gAC | G/D | Unchanged |
| MYO1C | 17-1384066-ACC-GCT | GgT/AgC | G/S | Unchanged |
| WDR81 | 17-1637073-CA-TG | cCA/cTG | P/L | Unchanged |
| OR1E2 | 17-3337056-CA-TG | TGc/CAc | C/H | Changed missense |
| ZZEF1 | 17-3981289-CA-TG | gTG/gCA | V/A | Unchanged |
| KIF1C | 17-4926881-CA-TG | cCA/cTG | P/L | Unchanged |
| ACAP1 | 17-7245370-CA-TG | cCA/cTG | P/L | Unchanged |
| AURKB | 17-8108330-CA-TG | aTG/aCA | M/T | Partially changed missense |
| DNAH9 | 17-11523081-CA-TG | CAa/TGa | Q/* | Unchanged |
| DNAH9 | 17-11648331-AC-GT | aAC/aGT | N/S | Unchanged |
| TBC1D26 | 17-15640814-CA-TG | CAc/TGc | H/C | Changed missense |
| TBC1D26 | 17-15640815-AC-GT | cAC/cGT | H/R | Unchanged |
| CCDC144A | 17-16593796-AGC-GGT | AgC/GgT | S/G | Unchanged |
| RP11-219A15.1 | 17-16593796-AGC-GGT | AgC/GgT | S/G | Unchanged |
| LLGL1 | 17-18137141-AGC-GGT | AgC/GgT | S/G | Unchanged |
| LGALS9 | 17-25975991-AC-TG | ACc/TGc | T/C | Changed missense |
| SEZ6 | 17-27284442-CA-TG | aTG/aCA | M/T | Partially changed missense |
| ANKRD13B | 17-27938606-CA-GT | aCA/aGT | T/S | Changed missense |
| LRRC37A | 17-44374709-CA-TG | cCA/cTG | P/L | Unchanged |
| TUBD1 | 17-57963536-CA-TG | aTG/aCA | M/T | Partially changed missense |
| TLK2 | 17-60683502-CA-TG | cCA/cTG | P/L | Unchanged |
| ACE | 17-61564051-CA-TG | gCA/gTG | A/V | Unchanged |
| APOH | 17-64216814-AC-GT | cGT/cAC | R/H | Unchanged |
| SLC16A6 | 17-66267530-CA-TG | cTG/cCA | L/P | Unchanged |
| ARSG | 17-66391231-CA-TG | cCA/cTG | P/L | Unchanged |
| COG1 | 17-71192872-CA-TG | gCA/gTG | A/V | Unchanged |
| ST6GALNAC1 | 17-74625685-CA-TG | gTG/gCA | V/A | Unchanged |
| ENGASE | 17-77078107-CGA-TGG | CgA/TgG | R/W | Rescued nonsense |
| RP11-1055B8.7 | 17-79409309-AC-GT | ACg/GTg | T/V | Changed missense |
| RP11-1055B8.7 | 17-79414307-ACC-GCT | AcC/GcT | T/A | Unchanged |
| CCDC57 | 17-80159713-CA-TG | cTG/cCA | L/P | Unchanged |
| HEXDC | 17-80391684-ATC-GTT | AtC/GtT | I/V | Unchanged |
| METRNL | 17-81043037-CCA-TCG | CcA/TcG | P/S | Unchanged |
| METRNL | 17-81043038-CA-TG | cCA/cTG | P/L | Unchanged |
| SMCHD1 | 18-2750476-CA-TG | gCA/gTG | A/V | Unchanged |
| TGIF1 | 18-3457538-CA-TG | cCA/cTG | P/L | Unchanged |
| SOGA2 | 18-8784611-CA-TG | CAg/TGg | Q/W | Rescued nonsense |
| NPC1 | 18-21124945-CA-GT | aTG/aAC | M/N | Changed missense |
| DSG2 | 18-29125853-CA-TG | aCA/aTG | T/M | Changed missense |
| MOCOS | 18-33780019-CA-TG | CAc/TGc | H/C | Changed missense |
| SLC14A1 | 18-43316537-CA-TG | cCA/cTG | P/L | Unchanged |
| SERPINB3 | 18-61325815-AC-GT | GTt/ACt | V/T | Changed missense |
| DOK6 | 18-67365667-CA-TG | aCA/aTG | T/M | Changed missense |
| ODF3L2 | 19-464309-CA-TG | gTG/gCA | V/A | Unchanged |
| GZMM | 19-548573-CA-GT | CAc/GTc | H/V | Changed missense |
| HMHA1 | 19-1085965-CA-TG | aCA/aTG | T/M | Changed missense |
| BTBD2 | 19-1997363-AGC-GGT | GcT/AcC | A/T | Unchanged |
| ZNF554 | 19-2834372-AC-GT | tAC/tGT | Y/C | Unchanged |
| APBA3 | 19-3752873-CA-TG | TGc/CAc | C/H | Changed missense |
| PIAS4 | 19-4029011-AC-GT | cAC/cGT | H/R | Unchanged |
| PLIN4 | 19-4511677-AGC-GGT | GcT/AcC | A/T | Unchanged |
| PLIN5 | 19-4525045-CA-TG | TGc/CAc | C/H | Changed missense |
| EMR1 | 19-6913706-AC-GT | ACg/GTg | T/V | Changed missense |
| CTD-2207O23.3 | 19-7528733-CA-TG | CAg/TGg | Q/W | Rescued nonsense |
| ARHGEF18 | 19-7528733-CA-TG | CAg/TGg | Q/W | Rescued nonsense |
| CTD-3193O13.9 | 19-7935583-CA-TG | tTG/tCA | L/S | Unchanged |
| CTD-3193O13.9 | 19-7936006-CA-TG | aTG/aCA | M/T | Partially changed missense |
| ACTL9 | 19-8808899-CA-TG | gTG/gCA | V/A | Unchanged |
| RAVER1 | 19-10439711-AC-GT | cGT/cAC | R/H | Unchanged |
| RGL3 | 19-11529440-AC-GT | gGT/gAC | G/D | Unchanged |
| ZNF440 | 19-11942362-AC-GT | aAC/aGT | N/S | Unchanged |
| MAST1 | 19-12985575-CA-TG | tCA/tTG | S/L | Unchanged |
| CYP4F11 | 19-16034713-CA-TG | TGc/CAc | C/H | Changed missense |
| CPAMD8 | 19-17111296-CA-TG | aTG/aCA | M/T | Partially changed missense |
| MYO9B | 19-17283655-ATC-GTT | AtC/GtT | I/V | Unchanged |
| MYO9B | 19-17316955-CA-TG | CAg/TGg | Q/W | Rescued nonsense |
| ANKLE1 | 19-17395002-CA-TG | CAg/TGg | Q/W | Rescued nonsense |
| TMEM221 | 19-17547291-CA-TG | aTG/aCA | M/T | Partially changed missense |
| MAP1S | 19-17837980-CA-TG | gCA/gTG | A/V | Unchanged |
| IFI30 | 19-18288067-CCA-TCG | CcA/TcG | P/S | Unchanged |
| KIAA1683 | 19-18378218-CA-TG | cTG/cCA | L/P | Unchanged |
| ATP4A | 19-36050968-CA-TG | gTG/gCA | V/A | Unchanged |
| ATP4A | 19-36050969-AC-GT | GTg/ACg | V/T | Changed missense |
| ZNF567 | 19-37210804-AC-GT | tAC/tGT | Y/C | Unchanged |
| RASGRP4 | 19-38905683-AC-GT | gGT/gAC | G/D | Unchanged |
| RYR1 | 19-38993546-AC-GT | cAC/cGT | H/R | Unchanged |
| IFNL4 | 19-39739170-CA-TG | TGc/CAc | C/H | Changed missense |
| SHKBP1 | 19-41096188-CA-TG | aCA/aTG | T/M | Changed missense |
| CYP2A6 | 19-41351304-CA-TG | aTG/aCA | M/T | Partially changed missense |
| CYP2A7 | 19-41383200-CA-TG | aTG/aCA | M/T | Partially changed missense |
| CYP2F1 | 19-41633926-CA-TG | cCA/cTG | P/L | Unchanged |
| AXL | 19-41743861-AAC-GAT | AaC/GaT | N/D | Unchanged |
| PSG11 | 19-43529025-AC-GT | GTa/ACa | V/T | Changed missense |
| TEX101 | 19-43922060-CA-TG | cCA/cTG | P/L | Unchanged |
| ZNF285 | 19-44891838-CA-TG | TGt/CAt | C/H | Changed missense |
| PVRL2 | 19-45389223-CA-TG | cCA/cTG | P/L | Unchanged |
| HIF3A | 19-46823701-CA-TG | CAg/TGg | Q/W | Rescued nonsense |
| GLTSCR2 | 19-48255803-CA-TG | gCA/gTG | A/V | Unchanged |
| GRIN2D | 19-48946404-CA-TG | cCA/cTG | P/L | Unchanged |
| KLK1 | 19-51323501-ATC-GTT | GaT/AaC | D/N | Unchanged |
| KLK3 | 19-51359502-CA-TG | gCA/gTG | A/V | Unchanged |
| SIGLEC5 | 19-52133292-AC-GT | GTt/ACt | V/T | Changed missense |
| ZNF534 | 19-52942445-CA-TG | CAc/TGc | H/C | Changed missense |
| ERVV-2 | 19-53554074-CA-TG | CAa/TGa | Q/* | Unchanged |
| VN1R2 | 19-53761764-ACC-GCT | AcC/GcT | T/A | Unchanged |
| LILRA6 | 19-54745906-CA-TG | aTG/aCA | M/T | Partially changed missense |
| LILRA4 | 19-54844856-CA-TG | TGg/CAg | W/Q | Rescued nonsense |
| LILRB4 | 19-55179216-CA-TG | gCA/gTG | A/V | Unchanged |
| PTPRH | 19-55708555-CAA-TAG | TtG/CtA | L | Unchanged |
| PTPRH | 19-55713614-ATC-GTT | GaT/AaC | D/N | Unchanged |
| ZNF628 | 19-55994652-CA-TG | CAg/TGg | Q/W | Rescued nonsense |
| CCDC106 | 19-56162774-AGC-GGT | AgC/GgT | S/G | Unchanged |
| NLRP13 | 19-56407380-ATC-TTG | GaT/CaA | D/Q | Changed missense |
| ZNF71 | 19-57133632-AC-GT | tAC/tGT | Y/C | Unchanged |
| ZSCAN4 | 19-58189713-CTA-TTG | CtA/TtG | L | Unchanged |
| SNTG2 | 2-1271230-ATC-GTT | AtC/GtT | I/V | Unchanged |
| TAF1B | 2-10045013-AC-GT | tAC/tGT | Y/C | Unchanged |
| MSGN1 | 2-17998095-CA-TG | CAc/TGc | H/C | Changed missense |
| EMILIN1 | 2-27306045-CA-TG | CAa/TGa | Q/* | Unchanged |
| CAD | 2-27460967-CA-TG | gCA/gTG | A/V | Unchanged |
| WDR43 | 2-29152526-CCA-GCT | CcA/GcT | P/A | Unchanged |
| FAM179A | 2-29240059-CA-TG | CAg/TGg | Q/W | Rescued nonsense |
| LTBP1 | 2-33447201-AC-GT | aAC/aGT | N/S | Unchanged |
| RASGRP3 | 2-33764176-AC-GT | ACg/GTg | T/V | Changed missense |
| ATL2 | 2-38604349-CCA-TCG | TgG/CgA | W/R | Rescued nonsense |
| SOCS5 | 2-46986415-CA-TG | aCA/aTG | T/M | Changed missense |
| MSH6 | 2-48018080-CA-TG | cCA/cTG | P/L | Unchanged |
| EML6 | 2-55040425-CA-TG | cCA/cTG | P/L | Unchanged |
| USP34 | 2-61575307-CA-TG | aTG/aCA | M/T | Partially changed missense |
| FAM161A | 2-62066927-AC-GT | tGT/tAC | C/Y | Unchanged |
| CEP68 | 2-65298838-CA-TG | aCA/aTG | T/M | Changed missense |
| LOXL3 | 2-74761469-CA-GT | TGt/ACt | C/T | Changed missense |
| HK2 | 2-75113656-AC-GT | aAC/aGT | N/S | Unchanged |
| IGKV3-15 | 2-89384829-AGC-GGT | GcT/AcC | A/T | Unchanged |
| ITPRIPL1 | 2-96993883-CA-TG | cCA/cTG | P/L | Unchanged |
| FAHD2B | 2-97755996-CCA-TCG | TgG/CgA | W/R | Rescued nonsense |
| ANKRD36 | 2-97909726-CA-TG | gCA/gTG | A/V | Unchanged |
| RFX8 | 2-102018907-CA-TG | aTG/aCA | M/T | Partially changed missense |
| TGFBRAP1 | 2-105924510-AC-GT | cGT/cAC | R/H | Unchanged |
| RANBP2 | 2-109374951-CA-TG | tCA/tTG | S/L | Unchanged |
| MERTK | 2-112705142-CA-TG | cCA/cTG | P/L | Unchanged |
| MERTK | 2-112765972-CA-TG | tCA/tTG | S/L | Unchanged |
| TUBA3D | 2-132237982-CA-TG | aCA/aTG | T/M | Changed missense |
| TUBA3D | 2-132238284-ACC-GCT | AcC/GcT | T/A | Unchanged |
| NCKAP5 | 2-133541574-AC-GT | GTc/ACc | V/T | Changed missense |
| THSD7B | 2-138417312-CA-TG | aCA/aTG | T/M | Changed missense |
| RIF1 | 2-152322332-AAC-GAT | AaC/GaT | N/D | Unchanged |
| COBLL1 | 2-165551659-CA-GT | TGg/ACg | W/T | Changed missense |
| TTN | 2-179518002-CA-TG | tTG/tCA | L/S | Unchanged |
| ZNF804A | 2-185802362-CA-TG | CAc/TGc | H/C | Changed missense |
| FSIP2 | 2-186678632-AC-GT | ACt/GTt | T/V | Changed missense |
| SLC40A1 | 2-190430177-AAC-GAT | GtT/AtC | V/I | Unchanged |
| CRYGB | 2-209010694-CA-GT | TGc/ACc | C/T | Changed missense |
| C2orf80 | 2-209036777-CA-TG | TGc/CAc | C/H | Changed missense |
| CPS1 | 2-211456637-ACC-GCT | AcC/GcT | T/A | Unchanged |
| TNS1 | 2-218674696-AC-GT | GTc/ACc | V/T | Changed missense |
| TNS1 | 2-218683153-CA-TG | TGg/CAg | W/Q | Rescued nonsense |
| RUFY4 | 2-218940395-CA-TG | CAa/TGa | Q/* | Unchanged |
| DNPEP | 2-220250146-CA-TG | gTG/gCA | V/A | Unchanged |
| TMEM198 | 2-220414015-AC-GT | gAC/gGT | D/G | Unchanged |
| COL4A3 | 2-228131169-AC-GT | cAC/cGT | H/R | Unchanged |
| SP140L | 2-231256811-CA-TG | gCA/gTG | A/V | Unchanged |
| UGT1A4 | 2-234627743-AC-GT | ACg/GTg | T/V | Changed missense |
| RAB17 | 2-238494741-CA-TG | gTG/gCA | V/A | Unchanged |
| AC079612.1 | 2-240500412-CA-TG | cCA/cTG | P/L | Unchanged |
| PRR21 | 2-240981486-CA-TG | TGc/CAc | C/H | Changed missense |
| PRR21 | 2-240982130-CA-TG | aTG/aCA | M/T | Partially changed missense |
| PRR21 | 2-240982200-CA-TG | TGt/CAt | C/H | Changed missense |
| PRR21 | 2-240982256-CA-TG | aTG/aCA | M/T | Partially changed missense |
| GPC1 | 2-241405478-AC-GT | gAC/gGT | D/G | Unchanged |
| CAPN10 | 2-241531477-CCA-TCG | CcA/TcG | P/S | Unchanged |
| AC104809.3 | 2-241898823-CA-TG | CAg/TGg | Q/W | Rescued nonsense |
| SDCBP2 | 20-1293008-CA-TG | gTG/gCA | V/A | Unchanged |
| SMOX | 20-4164282-CA-TG | aCA/aTG | T/M | Changed missense |
| PABPC1L | 20-43566786-CA-TG | tCA/tTG | S/L | Unchanged |
| FAM65C | 20-49214155-CA-TG | cTG/cCA | L/P | Unchanged |
| OGFR | 20-61444628-CA-TG | cCA/cTG | P/L | Unchanged |
| SLC17A9 | 20-61594679-AC-GT | aAC/aGT | N/S | Unchanged |
| RRP1B | 21-45107517-CA-TG | cCA/cTG | P/L | Unchanged |
| KRTAP10-6 | 21-46012363-CA-TG | aTG/aCA | M/T | Unchanged |
| COL18A1 | 21-46927488-CA-TG | aCA/aTG | T/M | Changed missense |
| AC008132.13 | 22-18834965-CA-TG | gCA/gTG | A/V | Unchanged |
| TBX1 | 22-19753960-CA-TG | gCA/gTG | A/V | Unchanged |
| P2RX6 | 22-21377275-AC-GT | ACc/GTc | T/V | Changed missense |
| IGLV3-10 | 22-23154570-CCA-TCG | CcA/TcG | P/S | Unchanged |
| IGLV3-1 | 22-23223438-AGC-TGG | AgC/TgG | S/W | Changed missense |
| THOC5 | 22-29913271-AC-GT | GTt/ACt | V/T | Changed missense |
| CABP7 | 22-30123750-AC-GT | aAC/aGT | N/S | Unchanged |
| SLC35E4 | 22-31032918-CCA-TCG | CcA/TcG | P/S | Unchanged |
| C22orf42 | 22-32554984-CA-TG | cTG/cCA | L/P | Unchanged |
| CSF2RB | 22-37333793-CA-TG | cCA/cTG | P/L | Unchanged |
| TRIOBP | 22-38122461-CA-TG | CAc/TGc | H/C | Changed missense |
| SLC16A8 | 22-38474695-CA-TG | gTG/gCA | V/A | Unchanged |
| MCHR1 | 22-41075694-AC-GT | aAC/aGT | N/S | Unchanged |
| SCUBE1 | 22-43610106-CGA-TGG | TcG/CcA | S/P | Unchanged |
| PRR5-ARHGAP8 | 22-45198008-CA-TG | cCA/cTG | P/L | Unchanged |
| ARHGAP8 | 22-45198008-CA-TG | cCA/cTG | P/L | Unchanged |
| CELSR1 | 22-46929691-CA-TG | TGc/CAc | C/H | Changed missense |
| ZBED4 | 22-50277881-CCA-TCG | CcA/TcG | P/S | Unchanged |
| ZBED4 | 22-50277882-CA-TG | cCA/cTG | P/L | Unchanged |
| MOV10L1 | 22-50582625-CA-TG | CAg/TGg | Q/W | Rescued nonsense |
| TUBGCP6 | 22-50664611-CA-TG | tTG/tCA | L/S | Unchanged |
| TRNT1 | 3-3189278-CA-TG | gCA/gTG | A/V | Unchanged |
| IL17RE | 3-9952705-CA-TG | aCA/aTG | T/M | Changed missense |
| IL17RC | 3-9969880-AC-GT | ACg/GTg | T/V | Changed missense |
| TATDN2 | 3-10302055-CA-TG | CAt/TGt | H/C | Changed missense |
| RP11-438J1.1 | 3-10302055-CA-TG | CAt/TGt | H/C | Changed missense |
| CAND2 | 3-12856855-CA-TG | CAg/TGg | Q/W | Rescued nonsense |
| FBLN2 | 3-13670536-ACC-GCT | AcC/GcT | T/A | Unchanged |
| TMEM43 | 3-14180704-AGC-GGT | AgC/GgT | S/G | Unchanged |
| RP11-434D12.1 | 3-14180704-AGC-GGT | AgC/GgT | S/G | Unchanged |
| FGD5 | 3-14862947-CA-TG | cCA/cTG | P/L | Unchanged |
| DLEC1 | 3-38163574-CA-TG | cCA/cTG | P/L | Unchanged |
| SCN5A | 3-38592406-ATC-GTT | GaT/AaC | D/N | Unchanged |
| ZBTB47 | 3-42700629-CA-TG | cCA/cTG | P/L | Unchanged |
| FAM198A | 3-43095100-CA-TG | CAg/TGg | Q/W | Rescued nonsense |
| PTPN23 | 3-47453607-AC-TG | aAC/aTG | N/M | Changed missense |
| CCDC71 | 3-49200626-CA-TG | TGg/CAg | W/Q | Rescued nonsense |
| DNAH1 | 3-52430697-CA-TG | CAt/TGt | H/C | Changed missense |
| ITIH1 | 3-52821010-CA-TG | CAg/TGg | Q/W | Rescued nonsense |
| ITIH4 | 3-52864635-AC-GT | cGT/cAC | R/H | Unchanged |
| DCP1A | 3-53326340-AC-GT | GTg/ACg | V/T | Changed missense |
| PTPRG | 3-62189526-CA-TG | gCA/gTG | A/V | Unchanged |
| SLC25A26 | 3-66419941-CA-TG | gCA/gTG | A/V | Unchanged |
| FRMD4B | 3-69246150-CAA-TAG | TtG/CtA | L | Unchanged |
| CEP97 | 3-101476757-CA-TG | gCA/gTG | A/V | Unchanged |
| KIAA2018 | 3-113373929-CA-TG | gTG/gCA | V/A | Unchanged |
| HEG1 | 3-124728625-CA-TG | aTG/aCA | M/T | Partially changed missense |
| IGSF10 | 3-151166872-AC-GT | aGT/aAC | S/N | Unchanged |
| EPHB3 | 3-184289151-CA-TG | aCA/aTG | T/M | Changed missense |
| EHHADH | 3-184910041-CA-TG | tTG/tCA | L/S | Unchanged |
| LPP | 3-188590444-CGA-TGG | CgA/TgG | R/W | Rescued nonsense |
| PYDC2 | 3-191179192-CA-TG | CAa/TGa | Q/* | Unchanged |
| CPN2 | 3-194062517-AGC-GGT | GcT/AcC | A/T | Unchanged |
| MUC4 | 3-195506674-AC-GT | GTt/ACt | V/T | Changed missense |
| MUC4 | 3-195507226-AC-GT | GTc/ACc | V/T | Changed missense |
| MUC4 | 3-195507274-AC-GT | GTc/ACc | V/T | Changed missense |
| MUC4 | 3-195511138-AC-GT | GTt/ACt | V/T | Changed missense |
| MUC4 | 3-195514401-AGC-GGT | GcT/AcC | A/T | Unchanged |
| UVSSA | 4-1377614-CA-TG | tCA/tTG | S/L | Unchanged |
| EVC | 4-5735119-AC-GT | gAC/gGT | D/G | Unchanged |
| SH3TC1 | 4-8242465-CA-TG | gCA/gTG | A/V | Unchanged |
| CPZ | 4-8602934-CA-TG | tCA/tTG | S/L | Unchanged |
| SLC2A9 | 4-10020693-CA-TG | TGg/CAg | W/Q | Rescued nonsense |
| TMEM165 | 4-56262372-CCA-TCG | CcA/TcG | P/S | Unchanged |
| MTHFD2L | 4-75040281-CA-GT | CAg/GTg | Q/V | Changed missense |
| FRAS1 | 4-79240063-AC-GT | gAC/gGT | D/G | Unchanged |
| FRAS1 | 4-79372929-AC-GT | cAC/cGT | H/R | Unchanged |
| PTPN13 | 4-87684030-CA-TG | cCA/cTG | P/L | Unchanged |
| GRID2 | 4-94344031-AC-GT | tAC/tGT | Y/C | Unchanged |
| NEIL3 | 4-178256958-AC-TG | gAC/gTG | D/V | Partially changed missense |
| TENM3 | 4-183715018-AC-GT | gAC/gGT | D/G | Unchanged |
| SORBS2 | 4-186541301-AC-GT | cGT/cAC | R/H | Unchanged |
| DUX4L4 | 4-191003179-AC-GT | ACg/GTg | T/V | Changed missense |
| SLC9A3 | 5-475103-CA-TG | TGc/CAc | C/H | Changed missense |
| LPCAT1 | 5-1467003-CA-TG | aTG/aCA | M/T | Partially changed missense |
| SRD5A1 | 5-6651969-CA-TG | cCA/cTG | P/L | Unchanged |
| PDZD2 | 5-32058114-CA-TG | tCA/tTG | S/L | Unchanged |
| MROH2B | 5-41048532-AC-GT | cGT/cAC | R/H | Unchanged |
| ZNF131 | 5-43175101-AGC-GGT | AgC/GgT | S/G | Unchanged |
| ARHGEF28 | 5-73144844-CA-TG | tCA/tTG | S/L | Unchanged |
| PCDHA8 | 5-140222344-CGA-TGG | CgA/TgG | R/W | Rescued nonsense |
| PCDHA11 | 5-140250423-CTA-TTG | CtA/TtG | L | Unchanged |
| PCDHA12 | 5-140256624-CTA-TTG | CtA/TtG | L | Unchanged |
| PCDHB5 | 5-140516859-AGC-GGT | AgC/GgT | S/G | Unchanged |
| PCDHB10 | 5-140572667-AC-GT | aAC/aGT | N/S | Unchanged |
| PCDHB15 | 5-140627275-CA-TG | gCA/gTG | A/V | Unchanged |
| PCDHGB1 | 5-140731407-CA-TG | aCA/aTG | T/M | Changed missense |
| TCERG1 | 5-145838642-CA-TG | CAg/TGg | Q/W | Rescued nonsense |
| FAT2 | 5-150901612-CA-TG | tTG/tCA | L/S | Unchanged |
| HAVCR1 | 5-156479508-CA-TG | cTG/cCA | L/P | Unchanged |
| C5orf60 | 5-179069467-CA-TG | TGg/CAg | W/Q | Rescued nonsense |
| TBC1D9B | 5-179297352-AC-GT | tGT/tAC | C/Y | Unchanged |
| MGAT1 | 5-180218667-CA-TG | cTG/cCA | L/P | Unchanged |
| NQO2 | 6-3015789-CA-TG | cCA/cTG | P/L | Unchanged |
| PPP1R3G | 6-5086069-CA-TG | gCA/gTG | A/V | Unchanged |
| NUP153 | 6-17688691-ATC-GTT | GaT/AaC | D/N | Unchanged |
| LRRC16A | 6-25435757-CA-TG | tCA/tTG | S/L | Unchanged |
| HIST1H2BA | 6-25727263-ATC-GTT | AtC/GtT | I/V | Unchanged |
| HIST1H1A | 6-26017673-CAA-TAG | TtG/CtA | L | Unchanged |
| OR2J2 | 6-29142064-ACC-GCT | AcC/GcT | T/A | Unchanged |
| C6orf15 | 6-31079893-CA-TG | gTG/gCA | V/A | Unchanged |
| RNF5 | 6-32148029-CCA-TCG | CcA/TcG | P/S | Unchanged |
| HLA-DQA1 | 6-32610494-CA-TG | CAa/TGa | Q/* | Unchanged |
| HLA-DQB1 | 6-32632687-ATC-GTT | GaT/AaC | D/N | Unchanged |
| ZNF76 | 6-35260502-AC-GT | cAC/cGT | H/R | Unchanged |
| FRS3 | 6-41739191-AC-TG | gGT/gCA | G/A | Unchanged |
| C6orf132 | 6-42075068-CA-TG | tTG/tCA | L/S | Unchanged |
| GPR115 | 6-47682671-ATC-GTT | AtC/GtT | I/V | Unchanged |
| FILIP1 | 6-76023772-AC-GT | aGT/aAC | S/N | Unchanged |
| NT5E | 6-86199233-AC-GT | ACg/GTg | T/V | Changed missense |
| RP3-422G23.4 | 6-138703761-CA-TG | cCA/cTG | P/L | Unchanged |
| ADGB | 6-147136243-CA-TG | CAc/TGc | H/C | Changed missense |
| IGF2R | 6-160453977-CA-TG | tCA/tTG | S/L | Unchanged |
| LPA | 6-160969628-CA-TG | aTG/aCA | M/T | Partially changed missense |
| FAM120B | 6-170627835-AC-GT | ACa/GTa | T/V | Changed missense |
| AC145676.2 | 7-228658-CA-TG | TGt/CAt | C/H | Changed missense |
| FAM20C | 7-299881-AAC-GAT | AaC/GaT | N/D | Unchanged |
| SUN1 | 7-897491-CA-TG | aCA/aTG | T/M | Changed missense |
| GPR146 | 7-1097894-CA-TG | cCA/cTG | P/L | Unchanged |
| MICALL2 | 7-1484572-AC-GT | gGT/gAC | G/D | Unchanged |
| DNAH11 | 7-21630981-CA-TG | gCA/gTG | A/V | Unchanged |
| AEBP1 | 7-44148552-CA-TG | aCA/aTG | T/M | Changed missense |
| NACAD | 7-45124285-CA-TG | gTG/gCA | V/A | Unchanged |
| ABCA13 | 7-48285165-CAA-TAG | CaA/TaG | Q/* | Unchanged |
| ZNF479 | 7-57193783-CA-TG | cTG/cCA | L/P | Unchanged |
| TYW1 | 7-66648155-AAC-GAT | AaC/GaT | N/D | Unchanged |
| TRIM74 | 7-72436651-CA-TG | TGg/CAg | W/Q | Rescued nonsense |
| TRIM50 | 7-72738748-CA-TG | TGg/CAg | W/Q | Rescued nonsense |
| STX1A | 7-73122923-ATC-GTT | GaT/AaC | D/N | Unchanged |
| WBSCR28 | 7-73280018-ACC-GCT | AcC/GcT | T/A | Unchanged |
| ABCB1 | 7-87179601-ACC-GCT | GgT/AgC | G/S | Unchanged |
| BAIAP2L1 | 7-97933601-CA-TG | tTG/tCA | L/S | Unchanged |
| MUC12 | 7-100646746-CA-TG | aCA/aTG | T/M | Changed missense |
| MUC17 | 7-100677585-CA-TG | tCA/tTG | S/L | Unchanged |
| MUC17 | 7-100682274-CA-TG | cCA/cTG | P/L | Unchanged |
| MUC17 | 7-100684749-CA-TG | cCA/cTG | P/L | Unchanged |
| ST7-OT4 | 7-116596696-CA-TG | CAt/TGt | H/C | Changed missense |
| CCDC136 | 7-128455767-CAA-GAT | CaA/GaT | Q/D | Changed missense |
| PLXNA4 | 7-131815310-ATC-GTT | GaT/AaC | D/N | Unchanged |
| PLXNA4 | 7-131883385-AC-TG | GTg/CAg | V/Q | Changed missense |
| CREB3L2 | 7-137600689-AC-GT | GTt/ACt | V/T | Changed missense |
| ATP6V0A4 | 7-138455987-CA-TG | gTG/gCA | V/A | Unchanged |
| TRBV7-4 | 7-142176377-CA-TG | TGc/CAc | C/H | Changed missense |
| CLCN1 | 7-143042836-AC-GT | gAC/gGT | D/G | Unchanged |
| OR2F1 | 7-143657800-CA-TG | aCA/aTG | T/M | Changed missense |
| OR2A14 | 7-143826695-AGC-TGG | AgC/TgG | S/W | Changed missense |
| KRBA1 | 7-149421772-CA-TG | CAt/TGt | H/C | Changed missense |
| SLC4A2 | 7-150771792-AC-GT | ACt/GTt | T/V | Changed missense |
| DPP6 | 7-154667627-CA-TG | cCA/cTG | P/L | Unchanged |
| AC021218.2 | 7-155755825-CA-TG | tCA/tTG | S/L | Unchanged |
| ARHGEF10 | 8-1900910-AC-GT | cAC/cGT | H/R | Unchanged |
| TNKS | 8-9564436-CA-TG | aCA/aTG | T/M | Changed missense |
| MTMR7 | 8-17270787-AC-GT | cGT/cAC | R/H | Unchanged |
| MTUS1 | 8-17612874-CA-TG | TGt/CAt | C/H | Changed missense |
| R3HCC1 | 8-23148929-CA-TG | aCA/aTG | T/M | Changed missense |
| DOCK5 | 8-25267621-CA-TG | cCA/cTG | P/L | Unchanged |
| MAK16 | 8-33356073-CA-TG | CAg/TGg | Q/W | Rescued nonsense |
| PROSC | 8-37623819-ATC-GTT | AtC/GtT | I/V | Unchanged |
| RSPO2 | 8-108970366-CA-TG | cTG/cCA | L/P | Unchanged |
| HHLA1 | 8-133089974-CA-TG | tTG/tCA | L/S | Unchanged |
| SLC45A4 | 8-142222374-ACC-GCT | GgT/AgC | G/S | Unchanged |
| GLIS3 | 9-4118206-CGA-TGG | TcG/CcA | S/P | Unchanged |
| RECK | 9-36105263-CA-TG | cCA/cTG | P/L | Unchanged |
| GRHPR | 9-37429813-CA-TG | gCA/gTG | A/V | Unchanged |
| OSTF1 | 9-77752508-ATC-GTT | AtC/GtT | I/V | Unchanged |
| FGD3 | 9-95780464-CA-TG | aCA/aTG | T/M | Changed missense |
| RAD23B | 9-110084388-CA-GT | aCA/aGT | T/S | Changed missense |
| RGS3 | 9-116346286-AC-GT | tAC/tGT | Y/C | Unchanged |
| DFNB31 | 9-117166245-CA-TG | gTG/gCA | V/A | Unchanged |
| DFNB31 | 9-117169032-CA-TG | aTG/aCA | M/T | Partially changed missense |
| DFNB31 | 9-117186677-AC-GT | gGT/gAC | G/D | Unchanged |
| OR1B1 | 9-125391407-CAA-TAG | TtG/CtA | L | Unchanged |
| WDR38 | 9-127618775-CA-TG | CAc/TGc | H/C | Changed missense |
| LRSAM1 | 9-130242166-AAC-GAT | AaC/GaT | N/D | Unchanged |
| ODF2 | 9-131235320-CA-TG | aCA/aTG | T/M | Changed missense |
| ASS1 | 9-133346225-AC-GT | cAC/cGT | H/R | Unchanged |
| LAMC3 | 9-133911597-CA-TG | gCA/gTG | A/V | Unchanged |
| DBH | 9-136518096-CA-TG | aCA/aTG | T/M | Changed missense |
| AL390778.1 | 9-138150587-CA-TG | cTG/cCA | L/P | Unchanged |
| AL390778.1 | 9-138151156-CA-TG | tTG/tCA | L/S | Unchanged |
| PPP1R26 | 9-138378856-AAC-GAT | AaC/GaT | N/D | Unchanged |
| ENTPD8 | 9-140332477-CA-TG | cTG/cCA | L/P | Unchanged |
| PNPLA7 | 9-140357971-CA-TG | TGg/CAg | W/Q | Rescued nonsense |
| PNPLA7 | 9-140396120-CA-TG | gTG/gCA | V/A | Unchanged |
| EHMT1 | 9-140611120-CA-GT | gCA/gGT | A/G | Unchanged |
| GYG2 | X-2779569-CA-TG | CAc/TGc | H/C | Changed missense |
| SHROOM2 | X-9859097-CA-TG | cCA/cTG | P/L | Unchanged |
| MAGEB16 | X-35820794-CA-TG | CAc/TGc | H/C | Changed missense |
| RBMXL3 | X-114425236-AC-GT | tAC/tGT | Y/C | Unchanged |
| CCDC160 | X-133379551-CTA-TTG | CtA/TtG | L | Unchanged |
| MAGEA4 | X-151092219-CA-TG | gCA/gTG | A/V | Unchanged |
| CSAG1 | X-151908844-AC-TG | tAC/tTG | Y/L | Rescued nonsense |
| ATP6AP1 | X-153657081-CGA-TGG | CgA/TgG | R/W | Rescued nonsense |

TABLE G
Specific targetable MNV mutations from the gnomAD database that can
be modelled with SPACE using Cas9 proteins with NGA PAM recognition.

| Gene_name | MNV | MNV_codons | MNV_AAs | MNV_Category |
|---|---|---|---|---|
| C1orf170 | 1-915420-CA-TG | gTG/gCA | V/A | Unchanged |
| C1orf170 | 1-915421-AC-GT | GTg/ACg | V/T | Changed missense |
| C1orf170 | 1-916548-CA-TG | TGg/CAg | W/Q | Rescued nonsense |
| ATAD3B | 1-1423266-CA-TG | gCA/gTG | A/V | Unchanged |
| C1orf222 | 1-1875859-ATC-GTT | GaT/AaC | D/N | Unchanged |
| PRKCZ | 1-1991013-CA-TG | cCA/cTG | P/L | Unchanged |
| MORN1 | 1-2268235-CA-TG | TGt/CAt | C/H | Changed missense |
| CCDC27 | 1-3669200-CA-TG | cCA/cTG | P/L | Unchanged |
| KCNAB2 | 1-6158561-CA-TG | tCA/tTG | S/L | Unchanged |
| THAP3 | 1-6693096-CA-TG | CAg/TGg | Q/W | Rescued nonsense |
| PER3 | 1-7890063-CA-TG | aCA/aTG | T/M | Changed missense |
| SLC45A1 | 1-8390494-CA-TG | cCA/cTG | P/L | Unchanged |
| H6PD | 1-9324669-CA-TG | tCA/tTG | S/L | Unchanged |
| MASP2 | 1-11090896-CA-TG | gTG/gCA | V/A | Unchanged |
| PLOD1 | 1-12024233-AAC-GAT | AaC/GaT | N/D | Unchanged |
| PRAMEF1 | 1-12855598-AC-TG | aAC/aTG | N/M | Changed missense |
| PRAMEF2 | 1-12921087-AC-TG | aAC/aTG | N/M | Changed missense |
| PRAMEF4 | 1-12942989-CA-TG | TGt/CAt | C/H | Changed missense |
| CLCNKB | 1-16375063-CA-GT | gCA/gGT | A/G | Unchanged |
| NBPF1 | 1-16916483-AC-GT | tGT/tAC | C/Y | Unchanged |
| UBR4 | 1-19433449-AAC-GAT | GtT/AtC | V/I | Unchanged |
| TMCO4 | 1-20020993-AC-GT | cGT/cAC | R/H | Unchanged |
| EPHA8 | 1-22919903-AC-GT | aAC/aGT | N/S | Unchanged |
| STPG1 | 1-24710404-CA-TG | aTG/aCA | M/T | Partially changed missense |
| AC114494.1 | 1-31902417-CA-TG | aTG/aCA | M/T | Partially changed missense |
| ZSCAN20 | 1-33960061-CA-TG | gCA/gTG | A/V | Unchanged |
| AGO1 | 1-36359668-CA-TG | cCA/cTG | P/L | Unchanged |
| CSF3R | 1-36937065-AC-GT | cGT/cAC | R/H | Unchanged |
| GRIK3 | 1-37267555-AC-GT | GTc/ACc | V/T | Changed missense |
| GUCA2B | 1-42619138-CA-TG | gCA/gTG | A/V | Unchanged |
| TIE1 | 1-43784954-CTA-TTG | CtA/TtG | L | Unchanged |
| MAST2 | 1-46500882-AC-GT | aAC/aGT | N/S | Unchanged |
| SLC1A7 | 1-53558270-AC-GT | cGT/cAC | R/H | Unchanged |
| MROH7-TTC4 | 1-55139740-CA-TG | CAc/TGc | H/C | Changed missense |
| MROH7 | 1-55139740-CA-TG | CAc/TGc | H/C | Changed missense |
| KANK4 | 1-62728783-CA-TG | gTG/gCA | V/A | Unchanged |
| AK5 | 1-77759577-CA-TG | gCA/gTG | A/V | Unchanged |
| SSX2IP | 1-85113228-CA-TG | TGt/CAt | C/H | Changed missense |
| CLCA2 | 1-86920902-CAA-TAG | CaA/TaG | Q/* | Unchanged |
| ATP5F1 | 1-112002206-AC-GT | cAC/cGT | H/R | Unchanged |
| PTPN22 | 1-114377567-CA-TG | TGg/CAg | W/Q | Rescued nonsense |
| IGSF3 | 1-117142868-CA-TG | TGg/CAg | W/Q | Rescued nonsense |
| NBPF16 | 1-148756446-AC-GT | aAC/aGT | N/S | Unchanged |
| CGN | 1-151502977-CA-TG | CAg/TGg | Q/W | Rescued nonsense |
| THEM5 | 1-151825919-CA-TG | aTG/aCA | M/T | Partially changed missense |
| FLG | 1-152277363-CCA-TCG | TgG/CgA | W/R | Rescued nonsense |
| FLG | 1-152279715-CA-TG | gTG/gCA | V/A | Unchanged |
| FLG | 1-152279919-CA-TG | tTG/tCA | L/S | Unchanged |
| FLG | 1-152280687-CA-TG | gTG/gCA | V/A | Unchanged |
| FLG | 1-152283333-CA-TG | gTG/gCA | V/A | Unchanged |
| FLG | 1-152284170-CCA-TCG | TgG/CgA | W/R | Rescued nonsense |
| FLG | 1-152285553-CA-TG | gTG/gCA | V/A | Unchanged |
| FLG | 1-152286126-AC-GT | cGT/cAC | R/H | Unchanged |
| LCE1D | 1-152770506-AC-GT | cAC/cGT | H/R | Unchanged |
| ADAMTS4 | 1-161168004-CGA-TGG | TcG/CcA | S/P | Unchanged |
| FCGR2A | 1-161476204-CA-TG | CAg/TGg | Q/W | Rescued nonsense |
| ATF6 | 1-161928326-CA-TG | tCA/tTG | S/L | Unchanged |
| POGK | 1-166818565-CA-TG | gCA/gTG | A/V | Unchanged |
| FMO2 | 1-171168584-CA-TG | tCA/tTG | S/L | Unchanged |
| SUCO | 1-172558523-CA-TG | cCA/cTG | P/L | Unchanged |
| QSOX1 | 1-180145087-CA-TG | aCA/aTG | T/M | Changed missense |
| SMG7 | 1-183510142-AC-GT | cAC/cGT | H/R | Unchanged |
| SWT1 | 1-185171868-CA-TG | CAc/TGc | H/C | Changed missense |
| HMCN1 | 1-186101538-CA-TG | CAa/TGa | Q/* | Unchanged |
| IGFN1 | 1-201175273-CA-TG | CAg/TGg | Q/W | Rescued nonsense |
| ATP2B4 | 1-203652443-CA-TG | tCA/tTG | S/L | Unchanged |
| CR1 | 1-207753620-CA-TG | CAt/TGt | H/C | Changed missense |
| CAMK1G | 1-209782342-CA-TG | cCA/cTG | P/L | Unchanged |
| TRAF5 | 1-211533351-CA-TG | gCA/gTG | A/V | Unchanged |
| HHIPL2 | 1-222721288-CA-TG | tTG/tCA | L/S | Unchanged |
| EPHX1 | 1-226026405-CA-TG | CAt/TGt | H/C | Changed missense |
| OBSCN | 1-228402120-CA-TG | CAg/TGg | Q/W | Rescued nonsense |
| OBSCN | 1-228433216-CA-TG | aCA/aTG | T/M | Changed missense |
| OBSCN | 1-228465346-AAC-GAT | AaC/GaT | N/D | Unchanged |
| OBSCN | 1-228480281-CA-TG | aCA/aTG | T/M | Changed missense |
| OBSCN | 1-228503676-CA-TG | CAc/TGc | H/C | Changed missense |
| OBSCN | 1-228503677-AC-GT | cAC/cGT | H/R | Unchanged |
| OBSCN | 1-228560138-CA-TG | tCA/tTG | S/L | Unchanged |
| OR2L8 | 1-248112835-CA-TG | CAc/TGc | H/C | Changed missense |
| OR2M3 | 1-248366682-AC-GT | ACa/GTa | T/V | Changed missense |
| OR2T12 | 1-248458735-CA-TG | TGg/CAg | W/Q | Rescued nonsense |
| OR2T3 | 1-248637064-CA-TG | cCA/cTG | P/L | Unchanged |
| OR14I1 | 1-248845498-CA-TG | gTG/gCA | V/A | Unchanged |
| AKR1E2 | 10-487291O-AC-GT | gAC/gGT | D/G | Unchanged |
| AKR1C1 | 10-5010571-CA-TG | aCA/aTG | T/M | Changed missense |
| ITGA8 | 10-15573050-AC-GT | GTa/ACa | V/T | Changed missense |
| PTER | 10-16547056-CAA-TAG | CaA/TaG | Q/* | Unchanged |
| NEBL | 10-21106620-AC-GT | GTa/ACa | V/T | Changed missense |
| KIAA1217 | 10-24813326-CA-TG | aCA/aTG | T/M | Changed missense |
| ASAH2C | 10-48003991-AC-GT | tGT/tAC | C/Y | Unchanged |
| NUTM2E | 10-81608367-CA-TG | CAg/TGg | Q/W | Rescued nonsense |
| CEP55 | 10-95262981-AC-GT | ACa/GTa | T/V | Changed missense |
| PYROXD2 | 10-100148175-CA-TG | aTG/aCA | M/T | Partially changed missense |
| HPS1 | 10-100202987-AC-GT | GTc/ACc | V/T | Changed missense |
| CWF19L1 | 10-102016043-AC-GT | tGT/tAC | C/Y | Unchanged |
| NRAP | 10-115411613-CA-TG | gTG/gCA | V/A | Unchanged |
| NRAP | 10-115411614-AC-GT | GTg/ACg | V/T | Changed missense |
| WDR11 | 10-122649481-CA-TG | gCA/gTG | A/V | Unchanged |
| CTBP2 | 10-126715153-CA-TG | cTG/cCA | L/P | Unchanged |
| JAKMIP3 | 10-133961493-AC-GT | cAC/cGT | H/R | Unchanged |
| LRRC27 | 10-134161631-CCA-TCG | CcA/TcG | P/S | Unchanged |
| LRRC27 | 10-134161632-CA-TG | cCA/cTG | P/L | Unchanged |
| CYP2E1 | 10-135342049-AC-TG | cAC/cTG | H/L | Partially changed missense |
| SCGB1C1 | 11-193721-CA-TG | aCA/aTG | T/M | Changed missense |
| PSMD13 | 11-244166-AC-GT | aAC/aGT | N/S | Unchanged |
| ATHL1 | 11-290815-CA-TG | gCA/gTG | A/V | Unchanged |
| ANO9 | 11-428384-CA-TG | TGc/CAc | C/H | Changed missense |
| MUC5B | 11-1264361-CA-TG | aCA/aTG | T/M | Changed missense |
| OR51B2 | 11-5345169-CA-TG | TGt/CAt | C/H | Changed missense |
| OR51B5 | 11-5364692-AGC-GGT | GcT/AcC | A/T | Unchanged |
| OR52H1 | 11-5566029-CA-TG | TGc/CAc | C/H | Changed missense |
| OR52L1 | 11-6007271-CA-TG | TGg/CAg | W/Q | Rescued nonsense |
| OR52L1 | 11-6007679-CA-TG | TGc/CAc | C/H | Changed missense |
| DNHD1 | 11-6550003-CA-TG | CAg/TGg | Q/W | Rescued nonsense |
| ZNF214 | 11-7022159-CA-TG | TGt/CAt | C/H | Changed missense |
| OR10A6 | 11-7949349-CA-TG | cTG/cCA | L/P | Unchanged |
| TUB | 11-8122089-AGC-GGT | AgC/GgT | S/G | Unchanged |
| SWAP70 | 11-9754220-AC-GT | aAC/aGT | N/S | Unchanged |
| SAA4 | 11-18253176-CA-TG | TGc/CAc | C/H | Changed missense |
| SAA2 | 11-18267026-AC-GT | cGT/cAC | R/H | Unchanged |
| SLC6A5 | 11-20639399-CA-TG | tCA/tTG | S/L | Unchanged |
| SLC5A12 | 11-26702721-CA-TG | gTG/gCA | V/A | Unchanged |
| NAT10 | 11-34130341-CA-TG | tCA/tTG | S/L | Unchanged |
| OR4S1 | 11-48328642-AAC-GAT | AaC/GaT | N/D | Unchanged |
| OR4C16 | 11-55339829-AC-GT | ACc/GTc | T/V | Changed missense |
| OR4C16 | 11-55339829-ACC-GCT | AcC/GcT | T/A | Unchanged |
| OR5L1 | 11-55579340-CA-TG | aCA/aTG | T/M | Changed missense |
| OR5F1 | 11-55761220-AC-GT | aGT/aAC | S/N | Unchanged |
| OR5J2 | 11-55944778-CA-TG | CAc/TGc | H/C | Changed missense |
| OR8U1 | 11-56143976-CA-TG | CAg/TGg | Q/W | Rescued nonsense |
| FAM111A | 11-58919812-CA-TG | gCA/gTG | A/V | Unchanged |
| OR4D10 | 11-59245494-CTA-TTG | CtA/TtG | L | Unchanged |
| NUDT22 | 11-63997328-CA-TG | CAg/TGg | Q/W | Rescued nonsense |
| PLCB3 | 11-64022458-CA-TG | cCA/cTG | P/L | Unchanged |
| TMEM262 | 11-64856499-CA-TG | TGg/CAg | W/Q | Rescued nonsense |
| PCNXL3 | 11-65385605-CA-TG | CAg/TGg | Q/W | Rescued nonsense |
| TBC1D10C | 11-67176897-CA-TG | tCA/tTG | S/L | Unchanged |
| AIP | 11-67258390-CA-TG | CAg/TGg | Q/W | Rescued nonsense |
| ALDH3B1 | 11-67789137-AC-GT | tAC/tGT | Y/C | Unchanged |
| IGHMBP2 | 11-68703959-AC-GT | ACg/GTg | T/V | Changed missense |
| PPFIA1 | 11-70221159-CA-TG | gCA/gTG | A/V | Unchanged |
| DHCR7 | 11-71146691-ATC-GTT | GaT/AaC | D/N | Unchanged |
| FOLR1 | 11-71907094-CA-TG | cCA/cTG | P/L | Unchanged |
| PDE2A | 11-72293602-ATC-GTT | GaT/AaC | D/N | Unchanged |
| XRRA1 | 11-74554492-AC-GT | GTc/ACc | V/T | Changed missense |
| FAT3 | 11-92531355-CA-TG | CAg/TGg | Q/W | Rescued nonsense |
| KDM4E | 11-94759058-CA-TG | CAg/TGg | Q/W | Rescued nonsense |
| ARHGAP42 | 11-100846935-CA-TG | cCA/cTG | P/L | Unchanged |
| ARHGAP42 | 11-100847049-CA-TG | tCA/tTG | S/L | Unchanged |
| BUD13 | 11-116627892-CA-TG | TGg/CAg | W/Q | Rescued nonsense |
| BACE1 | 11-117160346-CA-TG | TGc/CAc | C/H | Changed missense |
| CEP164 | 11-117252437-AC-GT | cAC/cGT | H/R | Unchanged |
| CEP164 | 11-117267883-CA-TG | CAg/TGg | Q/W | Rescued nonsense |
| BCL9L | 11-118771746-CA-TG | gTG/gCA | V/A | Unchanged |
| VPS11 | 11-118947633-AC-GT | tAC/tGT | Y/C | Unchanged |
| TRIM29 | 11-119999189-CAA-TAG | TtG/CtA | L | Unchanged |
| POU2F3 | 11-120175748-CA-TG | CAc/TGc | H/C | Changed missense |
| OR6T1 | 11-123814489-AC-GT | aGT/aAC | S/N | Unchanged |
| OR8B4 | 11-124294235-CA-TG | TGt/CAt | C/H | Changed missense |
| WNK1 | 12-939301-CA-TG | gCA/gTG | A/V | Unchanged |
| KLRC3 | 12-10573093-CCA-TCG | TgG/CgA | W/R | Rescued nonsense |
| SMCO3 | 12-14959469-CA-TG | TGc/CAc | C/H | Changed missense |
| SMCO2 | 12-27648785-CA-TG | gCA/gTG | A/V | Unchanged |
| CCDC65 | 12-49308283-CA-TG | CAc/TGc | H/C | Changed missense |
| KRT7 | 12-52631312-CA-TG | CAc/TGc | H/C | Changed missense |
| C12orf10 | 12-53700847-AC-GT | ACt/GTt | T/V | Changed missense |
| C12orf10 | 12-53700859-CA-TG | CAc/TGc | H/C | Changed missense |
| OR10P1 | 12-56031610-CA-TG | tCA/tTG | S/L | Unchanged |
| SDR9C7 | 12-57324078-AC-GT | cGT/cAC | R/H | Unchanged |
| C12orf55 | 12-97073470-CA-TG | CAt/TGt | H/C | Changed missense |
| MYBPC1 | 12-102069076-CA-TG | cCA/cTG | P/L | Unchanged |
| MYO1H | 12-109853348-CA-TG | cCA/cTG | P/L | Unchanged |
| IFT81 | 12-110618233-CGA-TGG | CgA/TgG | R/W | Rescued nonsense |
| DNAH10 | 12-124387585-CA-TG | gCA/gTG | A/V | Unchanged |
| SCARB1 | 12-125284748-AGC-GGT | GcT/AcC | A/T | Unchanged |
| TMEM132C | 12-129181809-CA-TG | aCA/aTG | T/M | Changed missense |
| EP400 | 12-132549300-AC-GT | ACa/GTa | T/V | Changed missense |
| C1QTNF9B | 13-24468328-CA-TG | TGt/CAt | C/H | Changed missense |
| RNF17 | 13-25419175-CA-TG | aCA/aTG | T/M | Changed missense |
| SLC46A3 | 13-29287551-AC-GT | GTt/ACt | V/T | Changed missense |
| FRY | 13-32676113-CA-TG | aCA/aTG | T/M | Changed missense |
| POSTN | 13-38156519-AC-GT | GTc/ACc | V/T | Changed missense |
| PROSER1 | 13-39587676-CA-TG | gTG/gCA | V/A | Unchanged |
| MRPS31 | 13-41323395-ATC-GTT | GaT/AaC | D/N | Unchanged |
| TSC22D1 | 13-45148904-AC-GT | GTa/ACa | V/T | Changed missense |
| PHF11 | 13-50C80845-CTA-TTG | CtA/TtG | L | Unchanged |
| ARL11 | 13-50205139-CA-TG | CAt/TGt | H/C | Changed missense |
| TMTC4 | 13-101287339-AC-GT | GTt/ACt | V/T | Changed missense |
| CDC16 | 13-115016035-CA-TG | aCA/aTG | T/M | Changed missense |
| POTEM | 14-20019783-AGC-GGT | GcT/AcC | A/T | Unchanged |
| RPGRIP1 | 14-21770680-CA-TG | cCA/cTG | P/L | Unchanged |
| CHD8 | 14-21871653-CA-TG | gTG/gCA | V/A | Unchanged |
| TRAV36DV7 | 14-22695128-ATC-GTT | AtC/GtT | I/V | Unchanged |
| AKAP6 | 14-33293021-CA-TG | gCA/gTG | A/V | Unchanged |
| MIA2 | 14-39703323-CA-TG | gCA/gTG | A/V | Unchanged |
| NIN | 14-51211072-AC-GT | tGT/tAC | C/Y | Unchanged |
| SYNE2 | 14-64522842-CA-TG | CAc/TGc | H/C | Changed missense |
| PLEKHH1 | 14-68042573-CA-TG | CAc/TGc | H/C | Changed missense |
| SLC8A3 | 14-70634087-AC-GT | cGT/cAC | R/H | Unchanged |
| VRK1 | 14-97299852-CA-TG | gCA/gTG | A/V | Unchanged |
| WDR25 | 14-100847706-CA-TG | CAt/TGt | H/C | Changed missense |
| KIF26A | 14-104643408-CA-TG | gCA/gTG | A/V | Unchanged |
| AHNAK2 | 14-105408726-ATC-TTG | GaT/CaA | D/Q | Changed missense |
| AHNAK2 | 14-105408810-CA-TG | cTG/cCA | L/P | Unchanged |
| IGHV1-8 | 14-106539280-AC-GT | aGT/aAC | S/N | Unchanged |
| IGHV3-43 | 14-106926317-ACC-TCG | GgT/CgA | G/R | Unchanged |
| GOLGA6L2 | 15-23688942-CCA-TCG | TgG/CgA | W/R | Rescued nonsense |
| GOLGA6L2 | 15-23688943-CA-TG | TGg/CAg | W/Q | Rescued nonsense |
| CHRFAM7A | 15-30659682-AC-GT | GTg/ACg | V/T | Changed missense |
| TRPM1 | 15-31369122-CA-TG | aTG/aCA | M/T | Partially changed missense |
| FMN1 | 15-33357261-CA-TG | cTG/cCA | L/P | Unchanged |
| RYR3 | 15-34113535-CA-TG | gCA/gTG | A/V | Unchanged |
| FSIP1 | 15-39910430-CA-TG | TGt/CAt | C/H | Changed missense |
| EIF2AK4 | 15-40260064-CAA-GAT | CaA/GaT | Q/D | Changed missense |
| SPINT1 | 15-41137111-AC-GT | aAC/aGT | N/S | Unchanged |
| MGA | 15-42026763-AC-GT | gAC/gGT | D/G | Unchanged |
| STRC | 15-43900291-AC-TG | GTt/CAt | V/H | Changed missense |
| DUOX1 | 15-45444517-CA-TG | aCA/aTG | T/M | Changed missense |
| TPM1 | 15-63354415-AC-GT | tAC/tGT | Y/C | Unchanged |
| TRIP4 | 15-64706331-CA-TG | cCA/cTG | P/L | Unchanged |
| KIF23 | 15-69728947-CCA-TCG | CcA/TcG | P/S | Unchanged |
| KIF23 | 15-69728948-CA-TG | cCA/cTG | P/L | Unchanged |
| C15orf39 | 15-75498547-CA-TG | gCA/gTG | A/V | Unchanged |
| TMED3 | 15-79614417-CA-TG | gCA/gTG | A/V | Unchanged |
| IL16 | 15-81592801-CA-TG | aCA/aTG | T/M | Changed missense |
| AGBL1 | 15-87217612-CA-TG | CAa/TGa | Q/* | Unchanged |
| UNC45A | 15-91488184-AC-GT | ACc/GTc | T/V | Changed missense |
| LRRK1 | 15-101598312-ACC-GCT | AcC/GcT | T/A | Unchanged |
| CHTF18 | 16-842449-AC-GT | aAC/aGT | N/S | Unchanged |
| CACNA1H | 16-1261218-CA-TG | tCA/tTG | S/L | Unchanged |
| CACNA1H | 16-1261221-CA-TG | tCA/tTG | S/L | Unchanged |
| ZNF598 | 16-2049639-CA-TG | aTG/aCA | M/T | Partially changed missense |
| E4F1 | 16-2285355-ATC-GTT | AtC/GtT | I/V | Unchanged |
| OR2C1 | 16-3405993-CA-TG | tCA/tTG | S/L | Unchanged |
| VASN | 16-4431201-CA-TG | aCA/aTG | T/M | Changed missense |
| ABAT | 16-8839953-CA-TG | CAg/TGg | Q/W | Rescued nonsense |
| TNFRSF17 | 16-12060163-AC-GT | aAC/aGT | N/S | Unchanged |
| NPIPA7 | 16-16481609-CA-TG | aCA/aTG | T/M | Changed missense |
| EEF2K | 16-22269866-CA-TG | CAa/TGa | Q/* | Unchanged |
| APOBR | 16-28508446-CA-TG | gCA/gTG | A/V | Unchanged |
| QPRT | 16-29708350-AC-GT | ACt/GTt | T/V | Changed missense |
| TAOK2 | 16-29998199-CA-TG | aCA/aTG | T/M | Changed missense |
| ZNF423 | 16-49672520-AC-GT | cGT/cAC | R/H | Unchanged |
| CNGB1 | 16-57957215-CA-TG | gTG/gCA | V/A | Unchanged |
| TEPP | 16-58019140-AC-GT | tAC/tGT | Y/C | Unchanged |
| WWP2 | 16-69970328-CA-TG | aGA/aTG | T/M | Changed missense |
| HP | 16-72094490-CAA-GAT | CaA/GaT | Q/D | Changed missense |
| PKD1L2 | 16-81213377-CA-TG | cTG/cCA | L/P | Unchanged |
| ZNF469 | 16-88505634-CA-TG | cCA/cTG | P/L | Unchanged |
| CDK10 | 16-89753128-CCA-GCT | CcA/GcT | P/A | Unchanged |
| KIF1C | 17-4926881-CA-TG | cCA/cTG | P/L | Unchanged |
| WSCD1 | 17-6014175-CA-TG | aCA/aTG | T/M | Changed missense |
| ALOX12 | 17-6902759-CA-TG | CAg/TGg | Q/W | Rescued nonsense |
| AURKB | 17-8108330-CA-TG | aTG/aCA | M/T | Partially changed missense |
| DNAH9 | 17-11523081-CA-TG | CAa/TGa | Q/* | Unchanged |
| DNAH9 | 17-11648331-AC-GT | aAC/aGT | N/S | Unchanged |
| TBC1D26 | 17-15640814-CA-TG | CAc/TGc | H/C | Changed missense |
| TBC1D26 | 17-15640815-AC-GT | cAC/cGT | H/R | Unchanged |
| CCDC144A | 17-16593796-AGC-GGT | AgC/GgT | S/G | Unchanged |
| RP11-219A15.1 | 17-16593796-AGC-GGT | AgC/GgT | S/G | Unchanged |
| NF1 | 17-29556425-CA-TG | cCA/cTG | P/L | Unchanged |
| ARHGAP23 | 17-36646385-CA-TG | gCA/gTG | A/V | Unchanged |
| KRTAP4-16P | 17-39258040-AC-GT | GTc/ACc | V/T | Changed missense |
| KRT36 | 17-39646021-AC-GT | cGT/cAC | R/H | Unchanged |
| NBR1 | 17-41361959-CA-TG | CAt/TGt | H/C | Changed missense |
| SPPL2C | 17-43923897-CA-TG | tCA/tTG | S/L | Unchanged |
| LRRC37A | 17-44374709-CA-TG | cCA/cTG | P/L | Unchanged |
| TRIM37 | 17-57094674-CA-GT | TGt/ACt | C/T | Changed missense |
| SMG8 | 17-57287453-CA-TG | gCA/gTG | A/V | Unchanged |
| MED13 | 17-60059990-CA-GT | TGt/ACt | C/T | Changed missense |
| TLK2 | 17-60683502-CA-TG | cCA/cTG | P/L | Unchanged |
| ACE | 17-61564051-CA-TG | gCA/gTG | A/V | Unchanged |
| ABCA10 | 17-67178315-CA-TG | aTG/aCA | M/T | Partially changed missense |
| TTYH2 | 17-72248482-CA-TG | gCA/gTG | A/V | Unchanged |
| CD300A | 17-72469957-CA-TG | cCA/cTG | P/L | Unchanged |
| CD300LF | 17-72700943-AC-GT | GTc/ACc | V/T | Changed missense |
| QRICH2 | 17-74289704-CA-TG | tTG/1CA | L/S | Unchanged |
| ENCASE | 17-77078107-CGA-TGG | CgA/TgG | R/W | Rescued nonsense |
| RP11-1055B8.7 | 17-79414307-ACC-GCT | AcC/GcT | T/A | Unchanged |
| SMCHD1 | 18-2740713-CA-TG | aCA/aTG | T/M | Changed missense |
| TXNDC2 | 18-9887551-ATC-GTT | AtC/GtT | I/V | Unchanged |
| NPC1 | 18-21124945-CA-GT | aTG/aAC | M/N | Changed missense |
| CDH2 | 18-25565082-AC-GT | cGT/cAC | R/H | Unchanged |
| TRAPPC8 | 18-29497572-CA-TG | tTG/tCA | L/S | Unchanged |
| MOCOS | 18-33780019-CA-TG | CAc/TGc | H/C | Changed missense |
| SERPINB4 | 18-61305035-CA-TG | TGt/CAt | C/H | Changed missense |
| ADNP2 | 18-77893682-CA-TG | gCA/gTG | A/V | Unchanged |
| POLRMT | 19-621712-AGC-GGT | GcT/AcC | A/T | Unchanged |
| ARID3A | 19-929752-CA-TG | cCA/cTG | P/L | Unchanged |
| HMHA1 | 19-1085965-CA-TG | aCA/aTG | T/M | Changed missense |
| ZNF554 | 19-2834372-AC-GT | tAC/tGT | Y/C | Unchanged |
| ZNF557 | 19-7083628-CA-TG | tCA/tTG | S/L | Unchanged |
| CTD-3193O13.9 | 19-7934992-CA-TG | tTG/tCA | L/S | Unchanged |
| CTD-3193O13.9 | 19-7936006-CA-TG | aTG/aCA | M/T | Partially changed missense |
| MUC16 | 19-9017004-CA-TG | TGg/CAg | W/Q | Rescued nonsense |
| OR1M1 | 19-9204306-CA-TG | cCA/cTG | P/L | Unchanged |
| ZNF561 | 19-9721521-CA-TG | gTG/gCA | V/A | Unchanged |
| ZNF440 | 19-11942362-AC-GT | aAC/aGT | N/S | Unchanged |
| ZNF442 | 19-12461211-CA-TG | aTG/aCA | M/T | Partially changed missense |
| CYP4F11 | 19-16034713-CA-TG | TGc/CAc | C/H | Changed missense |
| ANKLE1 | 19-17395002-CA-TG | CAg/TGg | Q/W | Rescued nonsense |
| ANO8 | 19-17438642-AAC-GAT | GtT/AtC | V/I | Unchanged |
| ZNF85 | 19-21117760-AC-GT | ACt/GTt | T/V | Changed missense |
| ZNF99 | 19-22952110-CA-TG | TGg/CAg | W/Q | Rescued nonsense |
| FXYD5 | 19-35649280-CA-TG | cCA/cTG | P/L | Unchanged |
| ZNF567 | 19-37210804-AC-GT | tAC/tGT | Y/C | Unchanged |
| ZNF568 | 19-37441979-CA-TG | CAa/TGa | Q/* | Unchanged |
| ZNF527 | 19-37879588-CA-TG | CAt/TGt | H/C | Changed missense |
| SHKBP1 | 19-41096188-CA-TG | aCA/aTG | T/M | Changed missense |
| CYP2A13 | 19-41600231-CA-TG | aCA/aTG | T/M | Changed missense |
| PSG5 | 19-43679410-AGC-GGT | GcT/AcC | A/T | Unchanged |
| PSG5 | 19-43680299-CA-TG | cTG/cCA | L/P | Unchanged |
| TEX101 | 19-43922060-CA-TG | cCA/cTG | P/L | Unchanged |
| ZNF224 | 19-44610842-AC-GT | ACg/GTg | T/V | Changed missense |
| ZNF285 | 19-44891849-AC-GT | aGT/aAC | S/N | Unchanged |
| PVRL2 | 19-45389223-CA-TG | cCA/cTG | P/L | Unchanged |
| HIF3A | 19-46823701-CA-TG | CAg/TGg | Q/W | Rescued nonsense |
| PRKD2 | 19-47177913-AC-GT | GTg/ACg | V/T | Changed missense |
| FUT2 | 19-49206416-CA-TG | gCA/gTG | A/V | Unchanged |
| KCNA7 | 19-49575618-ACC-GCT | GgT/AgC | G/S | Unchanged |
| KLK3 | 19-51359502-CA-TG | gCA/gTG | A/V | Unchanged |
| SIGLEC5 | 19-52133292-AC-GT | GTt/ACt | V/T | Changed missense |
| ZNF701 | 19-53085565-AC-TG | ACt/TGt | T/C | Changed missense |
| LILRB2 | 19-54782406-AC-GT | cGT/cAC | R/H | Unchanged |
| LILRA4 | 19-54844856-CA-TG | TGg/CAg | W/Q | Rescued nonsense |
| LILRA1 | 19-55105726-CA-TG | aCA/aTG | T/M | Changed missense |
| LILRB1 | 19-55144184-AGC-GGT | AgC/GgT | S/G | Unchanged |
| LILRB4 | 19-55179216-CA-TG | gCA/gTG | A/V | Unchanged |
| SBK2 | 19-56047447-CA-TG | TGc/CAc | C/H | Changed missense |
| NLRP13 | 19-56407380-ATC-TTG | GaT/CaA | D/Q | Changed missense |
| ZNF460 | 19-57802805-AC-GT | aAC/aGT | N/S | Unchanged |
| ZNF749 | 19-57955243-CA-TG | CAg/TGg | Q/W | Rescued nonsense |
| SNTG2 | 2-1271230-ATC-GTT | AtC/GtT | I/V | Unchanged |
| CMPK2 | 2-7005369-AC-GT | tGT/tAC | C/Y | Unchanged |
| DDX1 | 2-15747392-AC-GT | aAC/aGT | N/S | Unchanged |
| FAM179A | 2-29226511-CA-TG | CAg/TGg | Q/W | Rescued nonsense |
| FAM179A | 2-29240059-CA-TG | CAg/TGg | Q/W | Rescued nonsense |
| TTC27 | 2-32855613-CTA-TTG | CtA/TtG | L | Unchanged |
| CYP1B1 | 2-38298150-ATC-GTT | GaT/AaC | D/N | Unchanged |
| CYP1B1 | 2-38298202-AC-TG | GTg/CAg | V/Q | Changed missense |
| ERLEC1 | 2-54035445-CAA-TAG | CaA/TaG | Q/* | Unchanged |
| EML6 | 2-55040425-CA-TG | cCA/cTG | P/L | Unchanged |
| FAM161A | 2-62066927-AC-GT | tGT/tAC | C/Y | Unchanged |
| CD207 | 2-71058834-CA-TG | gTG/gCA | V/A | Unchanged |
| HK2 | 2-75113656-AC-GT | aAC/aGT | N/S | Unchanged |
| VAMP8 | 2-85808735-CGA-TGG | CgA/TgG | R/W | Rescued nonsense |
| IGKV1-12 | 2-89339910-CCA-TCG | TgG/CgA | W/R | Rescued nonsense |
| IGKV2-24 | 2-89475830-CA-TG | aTG/aCA | M/T | Partially changed missense |
| ITPRIPL1 | 2-96993883-CA-TG | cCA/cTG | P/L | Unchanged |
| FAHD2B | 2-97755996-OCA-TCG | TgG/CgA | W/R | Rescued nonsense |
| ANKRD36 | 2-97909726-CA-TG | gCA/gTG | A/V | Unchanged |
| RFX8 | 2-102018907-CA-TG | aTG/aCA | M/T | Partially changed missense |
| MERTK | 2-112765972-CA-TG | tCA/tTG | S/L | Unchanged |
| GLI2 | 2-121747427-CCA-TCG | CcA/TcG | P/S | Unchanged |
| TUBA3E | 2-130949552-CA-TG | TGg/CAg | W/Q | Rescued nonsense |
| POTEI | 2-131220830-CA-TG | aTG/aCA | M/T | Partially changed missense |
| TUBA3D | 2-132237982-CA-TG | aCA/aTG | T/M | Changed missense |
| THSD7B | 2-138417312-CA-TG | aCA/aTG | T/M | Changed missense |
| RIF1 | 2-152322240-AC-TG | gAC/gTG | D/V | Partially changed missense |
| RIF1 | 2-152322332-AAC-GAT | AaC/GaT | N/D | Unchanged |
| NEB | 2-152483630-AGC-GGT | GcT/AcC | A/T | Unchanged |
| TANC1 | 2-160086691-CA-TG | aCA/aTG | T/M | Changed missense |
| SCN9A | 2-167099157-CA-TG | TGg/CAg | W/Q | Rescued nonsense |
| SLC40A1 | 2-190430177-AAC-GAT | GtT/AtC | V/I | Unchanged |
| MFSD6 | 2-191302004-AC-GT | ACa/GTa | T/V | Changed missense |
| CPO | 2-207833982-CA-TG | aCA/aTG | T/M | Changed missense |
| CRYGB | 2-209010694-CA-GT | TGc/ACc | C/T | Changed missense |
| C2orf80 | 2-209036777-CA-TG | TGc/CAc | C/H | Changed missense |
| CPS1 | 2-211456637-ACC-GCT | AcC/GcT | T/A | Unchanged |
| SPEG | 2-220355528-CA-TG | CAc/TGc | H/C | Changed missense |
| SP140L | 2-231256811-CA-TG | gCA/gTG | A/V | Unchanged |
| AC079612.1 | 2-240500412-CA-TG | cCA/cTG | P/L | Unchanged |
| OR6B2 | 2-240969311-CA-TG | TGt/CAt | C/H | Changed missense |
| PRR21 | 2-240981659-AC-GT | cGT/cAC | R/H | Unchanged |
| PRR21 | 2-240982200-CA-TG | TGt/CAt | C/H | Changed missense |
| PRR21 | 2-240982256-CA-TG | aTG/aCA | M/T | Partially changed missense |
| CAPN10 | 2-241531477-CCA-TCG | CcA/TcG | P/S | Unchanged |
| SIRPG | 20-1616205-CA-TG | gTG/gCA | V/A | Unchanged |
| SIRPG | 20-1616206-AC-GT | GTg/ACg | V/T | Changed missense |
| SIRPA | 20-1895819-AC-GT | ACt/GTt | T/V | Changed missense |
| PLCB4 | 20-9404443-AAC-GAT | AsC/GSsT | N/D | Unchanged |
| SPTLC3 | 20-13053034-CA-TG | tCA/tTG | S/L | Unchanged |
| CST5 | 20-23860177-CA-TG | TGt/CAt | C/H | Changed missense |
| CEP250 | 20-34057798-CA-TG | aCA/aTG | T/M | Changed missense |
| PABPC1L | 20-43566786-CA-TG | tCA/tTG | S/L | Unchanged |
| FAM65C | 20-49214155-CA-TG | cTG/cCA | L/P | Unchanged |
| PCK1 | 20-56137806-CA-TG | tCA/tTG | S/L | Unchanged |
| OGFR | 20-61444628-CA-TG | cCA/cTG | P/L | Unchanged |
| SYNJ1 | 21-34003930-AC-GT | GTt/ACt | V/T | Changed missense |
| MX1 | 21-42824660-CA-TG | gCA/gTG | A/V | Unchanged |
| TRPM2 | 21-45844750-CA-TG | CAa/TGa | Q/* | Unchanged |
| C21orf67 | 21-46355710-AC-GT | cGT/cAC | R/H | Unchanged |
| COL18A1 | 21-46927488-CA-TG | aCA/aTG | T/M | Changed missense |
| PCNT | 21-47841940-CA-TG | CAa/TGa | Q/* | Unchanged |
| PCNT | 21-47851752-CA-TG | CAg/TGg | Q/W | Rescued nonsense |
| PCNT | 21-47855875-CA-TG | aCA/aTG | T/M | Changed missense |
| POTEH | 22-16287337-AGC-GGT | GcT/AcC | A/T | Unchanged |
| BCL2L13 | 22-18209716-AAC-GAT | AaC/GaT | N/D | Unchanged |
| TSSK2 | 22-19119750-AC-GT | ACg/GTg | T/V | Changed missense |
| IGLV4-69 | 22-22385708-AC-TG | tAC/tTG | Y/L | Rescued nonsense |
| IGLV9-49 | 22-22698013-AAC-GAT | AaC/GaT | N/D | Unchanged |
| IGLV3-1 | 22-23223438-AGC-TGG | AgC/TgG | S/W | Changed missense |
| RGL4 | 22-24034287-CA-TG | CAg/TGg | Q/W | Rescued nonsense |
| PIWIL3 | 22-25145470-CA-TG | TGt/CAt | C/H | Changed missense |
| C22orf31 | 22-29456698-CA-TG | TGt/CAt | C/H | Changed missense |
| CSF2RB | 22-37333793-CA-TG | cCA/cTG | P/L | Unchanged |
| TRIOBP | 22-38122461-CA-TG | CAc/TGc | H/C | Changed missense |
| MCHR1 | 22-41075694-AC-GT | aAC/aGT | N/S | Unchanged |
| CYP2D6 | 22-42523942-CA-TG | TGc/CAc | C/H | Changed missense |
| PRR5-ARHGAP8 | 22-45198008-CA-TG | cCA/cTG | P/L | Unchanged |
| ARHGAP8 | 22-45198008-CA-TG | cCA/cTG | P/L | Unchanged |
| FBLN1 | 22-45923826-CA-TG | CAg/TGg | Q/W | Rescued nonsense |
| PKDREJ | 22-46652998-ATC-GTT | GaT/AaC | D/N | Unchanged |
| PKDREJ | 22-46656241-CA-TG | gTG/gCA | V/A | Unchanged |
| CELSR1 | 22-46929691-CA-TG | TGc/CAc | C/H | Changed missense |
| ZBED4 | 22-50277882-CA-TG | cCA/cTG | P/L | Unchanged |
| ZBED4 | 22-50278437-CA-TG | cCA/cTG | P/L | Unchanged |
| TUBGCP6 | 22-50664611-CA-TG | tTG/tCA | L/S | Unchanged |
| CHL1 | 3-391100-AC-GT | ACa/GTa | T/V | Changed missense |
| IL17RC | 3-9970030-CA-TG | CAa/TGa | Q/* | Unchanged |
| TATDN2 | 3-10302055-CA-TG | CAt/TGt | H/C | Changed missense |
| RP11-438J1.1 | 3-10302055-CA-TG | CAt/TGt | H/C | Changed missense |
| TATDN2 | 3-10312505-CTA-TTG | CtA/TtG | L | Unchanged |
| RP11-438J1.1 | 3-10312505-CTA-TTG | CtA/TtG | L | Unchanged |
| FBLN2 | 3-13670480-CA-TG | aCA/aTG | T/M | Changed missense |
| ZNF860 | 3-32031134-CA-TG | tCA/tTG | S/L | Unchanged |
| SCN5A | 3-38592406-ATC-GTT | GaT/AaQ | D/N | Unchanged |
| CX3CR1 | 3-39307254-AAC-GAT | GtT/AtC | V/I | Unchanged |
| FAM198A | 3-43074870-CA-TG | gCA/gTG | A/V | Unchanged |
| ZNF852 | 3-44540715-CA-TG | aTG/aCA | M/T | Partially changed missense |
| SACM1L | 3-45754684-CA-TG | cCA/cTG | P/L | Unchanged |
| PTPN23 | 3-47453607-AC-TG | aAC/aTG | N/M | Changed missense |
| PARP3 | 3-51978219-CA-TG | CAc/TGc | H/C | Changed missense |
| ALAS1 | 3-52237968-ATC-GTT | AtC/GtT | I/V | Unchanged |
| DCP1A | 3-53326340-AC-GT | GTg/ACg | V/T | Changed missense |
| CCDC66 | 3-56597952-ACC-GCT | AcC/GcT | T/A | Unchanged |
| PTPRG | 3-62189526-CA-TG | gCA/gTG | A/V | Unchanged |
| ZNF717 | 3-75787869-CA-TG | TGc/CAc | C/H | Changed missense |
| ZNF717 | 3-75790879-CA-TG | gTG/gCA | V/A | Unchanged |
| GUCA1C | 3-108639422-AC-GT | GTt/ACt | V/T | Changed missense |
| KIAA2018 | 3-113373929-CA-TG | gTG/gCA | V/A | Unchanged |
| AGTR1 | 3-148459883-CA-TG | cCA/cTG | P/L | Unchanged |
| KLHL24 | 3-183368890-AC-GT | cAC/cGT | H/R | Unchanged |
| LPP | 3-188590444-CGA-TGG | CgA/TgG | R/W | Rescued nonsense |
| PYDC2 | 3-191179192-CA-TG | CAa/TGa | Q/* | Unchanged |
| MUC4 | 3-195505944-CA-TG | gTG/gCA | V/A | Unchanged |
| MUC4 | 3-195506674-AC-GT | GTt/ACt | V/T | Changed missense |
| MUC4 | 3-195507144-CA-TG | gTG/gCA | V/A | Unchanged |
| MUC4 | 3-195507226-AC-GT | GTc/ACc | V/T | Changed missense |
| MUC4 | 3-195507274-AC-GT | GTc/ACc | V/T | Changed missense |
| MUC4 | 3-195509823-ACC-TCG | GgT/CgA | G/R | Unchanged |
| MUC4 | 3-195509832-CA-TG | gTG/gCA | V/A | Unchanged |
| MUC4 | 3-195509976-CA-TG | gTG/gCA | V/A | Unchanged |
| MUC4 | 3-195510024-CA-TG | gTG/gCA | V/A | Unchanged |
| MUC4 | 3-195511138-AC-GT | GTt/ACt | V/T | Changed missense |
| MUC4 | 3-195514401-AGC-GGT | GcT/AcC | A/T | Unchanged |
| MUC4 | 3-195514776-CA-TG | gTG/gCA | V/A | Unchanged |
| UVSSA | 4-1377614-CA-TG | tCA/tTG | S/L | Unchanged |
| RGS12 | 4-3318412-CA-TG | tCA/tTG | S/L | Unchanged |
| WFS1 | 4-6304086-CA-TG | tCA/tTG | S/L | Unchanged |
| SLC2A9 | 4-10020693-CA-TG | TGg/CAg | W/Q | Rescued nonsense |
| SCFD2 | 4-54011525-CA-TG | tTG/tCA | L/S | Unchanged |
| KIAA1211 | 4-57182757-AC-GT | gAC/gGT | D/G | Unchanged |
| LPHN3 | 4-62845489-CA-TG | cCA/cTG | P/L | Unchanged |
| UGT2B7 | 4-69972951-AC-GT | tAC/tGT | Y/C | Unchanged |
| AFP | 4-74318329-CA-TG | aCA/aTG | T/M | Changed missense |
| FRAS1 | 4-79240063-AC-GT | gAC/gGT | D/G | Unchanged |
| FRAS1 | 4-79372929-AC-GT | cAC/cGT | H/R | Unchanged |
| PTPN13 | 4-87671749-CA-TG | tCA/tTG | S/L | Unchanged |
| PTPN13 | 4-87684030-CA-TG | cCA/cTG | P/L | Unchanged |
| DSPP | 4-88535868-AC-GT | aAC/aGT | N/S | Unchanged |
| DSPP | 4-88536437-AAC-GAT | AaC/GaT | N/D | Unchanged |
| DSPP | 4-88536899-AAC-GAT | AaC/GaT | N/D | Unchanged |
| DSPP | 4-88537556-AAC-GAT | AaC/GaT | N/D | Unchanged |
| GRID2 | 4-94344031-AC-GT | tAC/tGT | Y/C | Unchanged |
| C4orf17 | 4-100443852-CA-TG | cCA/cTG | P/L | Unchanged |
| BANK1 | 4-102751276-CTA-TTG | CtA/TtG | L | Unchanged |
| SEC24B | 4-110384789-CA-TG | cCA/cTG | P/L | Unchanged |
| ANK2 | 4-114277480-CA-TG | gCA/gTG | A/V | Unchanged |
| UGT8 | 4-115544713-CA-TG | cCA/cTG | P/L | Unchanged |
| ADAM29 | 4-175899095-CA-TG | CAg/TGg | Q/W | Rescued nonsense |
| CDKN2AIP | 4-184367724-CA-TG | tCA/tTG | S/L | Unchanged |
| TRAPPC11 | 4-184600600-CA-TG | gCA/gTG | A/V | Unchanged |
| FAM149A | 4-187078784-CA-TG | CAc/TGc | H/C | Changed missense |
| SRD5A1 | 5-6651969-CA-TG | cCA/cTG | P/L | Unchanged |
| CDH12 | 5-21752056-ATC-GTT | GaT/AaC | D/N | Unchanged |
| PDZD2 | 5-32058114-CA-TG | tCA/tTG | S/L | Unchanged |
| AMACR | 5-33998882-CA-TG | tTG/tCA | L/S | Unchanged |
| ZNF131 | 5-43175101-AGC-GGT | AgC/GgT | S/G | Unchanged |
| RP11-45H22.3 | 5-54253580-CA-TG | TGt/CAt | C/H | Changed missense |
| MCCC2 | 5-70945074-CA-TG | gCA/gTG | A/V | Unchanged |
| ANKRD34B | 5-79855371-CA-TG | tTG/tCA | L/S | Unchanged |
| MSH3 | 5-80149980-CA-TG | CAg/TGg | Q/W | Rescued nonsense |
| ATG10 | 5-81549215-AC-GT | ACg/GTg | T/V | Changed missense |
| HSD17B4 | 5-118872183-AC-GT | ACa/GTa | T/V | Changed missense |
| IL13 | 5-131995963-CA-TG | CAg/TGg | Q/W | Rescued nonsense |
| APBB3 | 5-139941227-CA-TG | TGc/CAc | C/H | Changed missense |
| PCDHA8 | 5-140222344-CGA-TGG | CgA/TgG | R/W | Rescued nonsense |
| PCDHA12 | 5-140256624-CTA-TTG | CtA/TtG | L | Unchanged |
| PCDHB10 | 5-140572667-AC-GT | aAC/aGT | N/S | Unchanged |
| PCDHB15 | 5-140627275-CA-TG | gCA/gTG | A/V | Unchanged |
| TCOF1 | 5-149755420-CA-TG | tCA/tTG | S/L | Unchanged |
| GM2A | 5-150639409-ATC-GTT | AtC/GtT | I/V | Unchanged |
| FAT2 | 5-150901612-CA-TG | tTG/tCA | L/S | Unchanged |
| MRPL22 | 5-154346258-CA-TG | tCA/tTG | S/L | Unchanged |
| HAVCR1 | 5-156479508-CA-TG | cTG/cCA | L/P | Unchanged |
| WWC1 | 5-167894863-CA-TG | CAc/TGc | H/C | Changed missense |
| PRELID1 | 5-176733114-CGA-TGG | CgA/TgG | R/W | Rescued nonsense |
| MGAT1 | 5-180218667-CA-TG | cTG/cCA | L/P | Unchanged |
| BTNL8 | 5-180338368-AC-GT | ACg/GTg | T/V | Changed missense |
| NQO2 | 6-3015789-CA-TG | cCA/cTG | P/L | Unchanged |
| RREB1 | 6-7231769-CA-TG | cCA/cTG | P/L | Unchanged |
| LRRC16A | 6-25435757-CA-TG | tCA/tTG | S/L | Unchanged |
| MUC22 | 6-30993957-CA-TG | gCA/gTG | A/V | Unchanged |
| MUC22 | 6-31002452-AAC-GAT | AaC/GaT | N/D | Unchanged |
| C6orf15 | 6-31079893-CA-TG | gTG/gCA | V/A | Unchanged |
| CFB | 6-31914933-CGA-TGG | CgA/TgG | R/W | Rescued nonsense |
| CYP21A2 | 6-32006871-AC-GT | tAC/tGT | Y/C | Unchanged |
| RNF5 | 6-32148029-CCA-TCG | CcA/TcG | P/S | Unchanged |
| HLA-DRA | 6-32410986-CA-TG | aCA/aTG | T/M | Changed missense |
| ZNF76 | 6-35260502-AC-GT | cAC/cGT | H/R | Unchanged |
| KIF6 | 6-39325077-CA-TG | TGg/CAg | W/Q | Rescued nonsense |
| GPR111 | 6-47624298-CA-TG | CAt/TGt | H/C | Changed missense |
| C6orf57 | 6-71289188-CA-TG | CAg/TGg | Q/W | Rescued nonsense |
| DDX43 | 6-74125887-CA-TG | CAg/TGg | Q/W | Rescued nonsense |
| FILIP1 | 6-76023772-AC-GT | aGT/aAC | S/N | Unchanged |
| IRAK1BP1 | 6-79595096-CA-TG | gCA/gTG | A/V | Unchanged |
| NT5E | 6-86199233-AC-GT | ACg/GTg | T/V | Changed missense |
| TBC1D32 | 6-121625944-CA-TG | tTG/tCA | L/S | Unchanged |
| PTPRK | 6-128403745-AAC-GAT | GtT/AtC | V/I | Unchanged |
| ENPP3 | 6-131995312-CA-TG | cCA/cTG | P/L | Unchanged |
| VNN2 | 6-133072650-AC-GT | gGT/gAC | G/D | Unchanged |
| IL20RA | 6-137325846-AC-GT | GTa/ACa | V/T | Changed missense |
| GPR126 | 6-142758630-CA-TG | CAg/TGg | Q/W | Rescued nonsense |
| HIVEP2 | 6-143091262-CA-TG | cTG/cCA | L/P | Unchanged |
| IGF2R | 6-160453977-CA-TG | tCA/tTG | S/L | Unchanged |
| FAM120B | 6-170627803-AC-GT | gAC/gGT | D/G | Unchanged |
| FAM120B | 6-170627836-CA-TG | aCA/aTG | T/M | Changed missense |
| AC145676.2 | 7-228658-CA-TG | TGt/CAt | C/H | Changed missense |
| SUN1 | 7-897491-CA-TG | aCA/aTG | T/M | Changed missense |
| MICALL2 | 7-1484572-AC-GT | gGT/gAC | G/D | Unchanged |
| SDK1 | 7-4213974-CA-TG | CAt/TGt | H/C | Changed missense |
| MIOS | 7-7646686-CA-TG | aCA/aTG | T/M | Changed missense |
| DNAH11 | 7-21628196-CA-TG | CAg/TGg | Q/W | Rescued nonsense |
| DNAH11 | 7-21630981-CA-TG | gCA/gTG | A/V | Unchanged |
| FAM188B | 7-30915260-AAC-GAT | AaC/GaT | N/D | Unchanged |
| AQP1 | 7-30915260-AAC-GAT | AaC/GaT | N/D | Unchanged |
| FAM188B | 7-30915261-AC-GT | aAC/aGT | N/S | Unchanged |
| AQP1 | 7-30915261-AC-GT | aAC/aGT | N/S | Unchanged |
| CCDC129 | 7-31618007-CA-TG | CAt/TGt | H/C | Changed missense |
| AVL9 | 7-32613019-CA-TG | aCA/aTG | T/M | Changed missense |
| AEBP1 | 7-44148552-CA-TG | aCA/aTG | T/M | Changed missense |
| ABCA13 | 7-48285165-CAA-TAG | CaA/TaG | Q/* | Unchanged |
| TYW1 | 7-66660241-CA-TG | CAc/TGc | H/C | Changed missense |
| TYW1 | 7-66660242-AC-GT | cAC/cGT | H/R | Unchanged |
| RHBDD2 | 7-75513061-CA-TG | gCA/gTG | A/V | Unchanged |
| DTX2 | 7-76131645-AC-GT | ACa/GTa | T/V | Changed missense |
| DMTF1 | 7-86800350-CA-TG | gCA/gTG | A/V | Unchanged |
| ABCB1 | 7-87179601-ACC-GCT | GgT/AgC | G/S | Unchanged |
| FBXO24 | 7-100198385-AC-GT | ACg/GTg | T/V | Changed missense |
| MUC17 | 7-100677585-CA-TG | tCA/tTG | S/L | Unchanged |
| CUX1 | 7-101844850-CA-TG | cCA/cTG | P/L | Unchanged |
| NAPEPLD | 7-102743891-ATC-GTT | GaT/AaC | D/N | Unchanged |
| GPR85 | 7-112724525-ACC-GCT | GgT/AgC | G/S | Unchanged |
| PLXNA4 | 7-131883385-AC-TG | GTg/CAg | V/Q | Changed missense |
| CREB3L2 | 7-137600689-AC-GT | GTt/ACt | V/T | Changed missense |
| ATP6V0A4 | 7-138417790-CA-TG | aTG/aCA | M/T | Partially changed missense |
| TRBV6-9 | 7-142104527-CA-TG | gTG/gCA | V/A | Unchanged |
| TRBV7-4 | 7-142176377-CA-TG | TGc/CAc | C/H | Changed missense |
| CLCN1 | 7-143042836-AC-GT | gAC/gGT | D/G | Unchanged |
| EPHA1 | 7-143097099-CA-TG | gTG/gCA | V/A | Unchanged |
| ZNF775 | 7-150094851-AC-GT | ACg/GTg | T/V | Changed missense |
| DPP6 | 7-154667627-CA-TG | cCA/cTG | P/L | Unchanged |
| RNF32 | 7-156469189-CA-TG | gCA/gTG | A/V | Unchanged |
| WDR60 | 7-158672618-CA-TG | CAa/TGa | Q/* | Unchanged |
| NEIL2 | 8-11640783-CA-TG | cCA/cTG | P/L | Unchanged |
| DLC1 | 8-12957474-AC-GT | GTg/ACg | V/T | Changed missense |
| PCM1 | 8-17796382-AC-GT | aAC/aGT | N/S | Unchanged |
| PDLIM2 | 8-22442660-CA-TG | gCA/gTG | A/V | Unchanged |
| R3HCC1 | 8-23148929-CA-TG | aCA/aTG | T/M | Changed missense |
| DOCK5 | 8-25267621-CA-TG | cCA/cTG | P/L | Unchanged |
| ADRA1A | 8-26628027-CA-TG | TGc/CAc | C/H | Changed missense |
| DDHD2 | 8-38095661-AC-GT | ACg/GTg | T/V | Changed missense |
| PREX2 | 8-69143588-CA-TG | cCA/cTG | P/L | Unchanged |
| SBSPON | 8-73982160-CA-TG | TGg/CAg | W/Q | Rescued nonsense |
| ZFHX4 | 8-77765864-AC-GT | tAC/tGT | Y/C | Unchanged |
| PKHD1L1 | 8-110416812-AC-GT | tAC/tGT | Y/C | Unchanged |
| TBC1D31 | 8-124121797-CA-TG | gCA/gTG | A/V | Unchanged |
| HHLA1 | 8-133089974-CA-TG | tTG/tCA | L/S | Unchanged |
| ZFAT | 8-135614705-AC-GT | cGT/cAC | R/H | Unchanged |
| CYP11B2 | 8-143994265-CA-TG | gTG/gCA | V/A | Unchanged |
| FAM83H | 8-144808955-AC-TG | aGT/aCA | S/T | Partially changed missense |
| PLEC | 8-145011204-AGC-GGT | GcT/AcC | A/T | Unchanged |
| ADAMTSL1 | 9-18681820-CA-TG | aCA/aTG | T/M | Changed missense |
| TEK | 9-27202869-CA-TG | tCA/tTG | S/L | Unchanged |
| SPATA31A3 | 9-40702845-AC-GT | ACc/GTc | T/V | Changed missense |
| FGD3 | 9-95780464-CA-TG | aCA/aTG | T/M | Changed missense |
| TBC1D2 | 9-100972949-CA-TG | cTG/cCA | L/P | Unchanged |
| TBC1D2 | 9-100995720-CA-TG | tTG/tCA | L/S | Unchanged |
| INVS | 9-103015206-CTA-TTG | CtA/TtG | L | Unchanged |
| RAD23B | 9-110084388-CA-GT | aCA/aGT | T/S | Changed missense |
| RGS3 | 9-116346286-AC-GT | tAC/tGT | Y/C | Unchanged |
| DFNB31 | 9-117169032-CA-TG | aTG/aCA | M/T | Partially changed missense |
| WDR38 | 9-127618775-CA-TG | CAc/TGc | H/C | Changed missense |
| ODF2 | 9-131235320-CA-TG | aCA/aTG | T/M | Changed missense |
| PPP1R26 | 9-138378856-AAC-GAT | AsG/GsT | N/D | Unchanged |
| ENTPD8 | 9-140332477-CA-TG | cTG/cCA | L/P | Unchanged |
| PNPLA7 | 9-140357971-CA-TG | TGg/CAg | W/Q | Rescued nonsense |
| PNPLA7 | 9-140396120-CA-TG | gTG/gCA | V/A | Unchanged |
| PRKX | X-3631166-CA-TG | gTG/gCA | V/A | Unchanged |
| XAGE5 | X-52842212-CA-TG | CAg/TGg | Q/W | Rescued nonsense |
| TSPYL2 | X-53115057-AAC-GAT | AaC/GaT | N/D | Unchanged |
| MAGED2 | X-54836360-CA-TG | tCA/tTG | S/L | Unchanged |
| KIF4A | X-69516919-AC-TG | ACt/TGt | T/C | Changed missense |
| CCDC160 | X-133379551-CTA-TTG | CtA/TtG | L | Unchanged |
| SPANXN3 | X-142596981-AC-GT | GTa/ACa | V/T | Changed missense |
| MAGEA4 | X-151092219-CA-TG | gCA/gTG | A/V | Unchanged |
| CSAG1 | X-151908844-AC-TG | tAC/tTG | Y/L | Rescued nonsense |
| PLXNA3 | X-153694819-CA-TG | aCA/aTG | T/M | Changed missense |

TABLE H
Specific targetable MNV mutations from the gnomAD database that can
be modelled with SPACE using Cas9 proteins with NG PAM recognition.

| Gene_name | MNV | MNV_codons | MNV_AAs | MNV_category |
|---|---|---|---|---|
| C1orf170 | 1-911594-CA-TG | gTG/gCA | V/A | Unchanged |
| C1orf170 | 1-915225-CAA-TAG | TtG/CtA | L | Unchanged |
| C1orf170 | 1-915420-CA-TG | gTG/gCA | V/A | Unchanged |
| C1orf170 | 1-915421-AC-GT | GTg/ACg | V/T | Changed missense |
| C1orf170 | 1-916548-CA-TG | TGg/CAg | W/Q | Rescued nonsense |
| AGRN | 1-981930-CA-TG | tCA/tTG | S/L | Unchanged |
| ATAD3B | 1-1423266-CA-TG | gCA/gTG | A/V | Unchanged |
| C1orf222 | 1-1875859-ATC-GTT | GaT/AaC | D/N | Unchanged |
| PRKCZ | 1-1991013-CA-TG | cCA/cTG | P/L | Unchanged |
| MORN1 | 1-2268235-CA-TG | TGt/CAt | C/H | Changed missense |
| MMEL1 | 1-2526745-CA-TG | aTG/aCA | M/T | Partially changed missense |
| ARHGEF16 | 1-3383896-CCA-TCG | CcA/TcG | P/S | Unchanged |
| ARHGEF16 | 1-3383897-CA-TG | cCA/cTG | P/L | Unchanged |
| CCDC27 | 1-3669200-CA-TG | cCA/cTG | P/L | Unchanged |
| KCNAB2 | 1-6158561-CA-TG | tCA/tTG | S/L | Unchanged |
| CHD5 | 1-6184090-CGA-TGG | TcG/CcA | S/P | Unchanged |
| THAP3 | 1-6693096-CA-TG | CAg/TGg | Q/W | Rescued nonsense |
| PER3 | 1-7890063-CA-TG | aCA/aTG | T/M | Changed missense |
| SLC45A1 | 1-8390494-CA-TG | cCA/cTG | P/L | Unchanged |
| H6PD | 1-9324669-CA-TG | tCA/tTG | S/L | Unchanged |
| PEX14 | 1-10689923-AC-GT | gAC/gGT | D/G | Unchanged |
| MASP2 | 1-11090896-CA-TG | gTG/gCA | V/A | Unchanged |
| PLOD1 | 1-12024233-AAC-GAT | AaC/GaT | N/D | Unchanged |
| PRAMEF1 | 1-12855598-AC-TG | aAC/aTG | N/M | Changed missense |
| PRAMEF2 | 1-12921087-AC-TG | aAC/aTG | N/M | Changed missense |
| PRAMEF2 | 1-12921384-AC-GT | gAC/gGT | D/G | Unchanged |
| PRAMEF4 | 1-12939414-CA-TG | TGt/CAt | C/H | Changed missense |
| PRAMEF4 | 1-12942989-CA-TG | TGt/CAt | C/H | Changed missense |
| PRAMEF5 | 1-13365986-CA-TG | CAg/TGg | Q/W | Rescued nonsense |
| PRDM2 | 1-14143002-CA-TG | cCA/cTG | P/L | Unchanged |
| CLCNKB | 1-16375063-CA-GT | gCA/gGT | A/G | Unchanged |
| CLCNKB | 1-16378276-ATC-GTT | AtC/GtT | I/V | Unchanged |
| NBPF1 | 1-16916483-AC-GT | tGT/tAC | C/Y | Unchanged |
| ARHGEF10L | 1-18023364-AC-GT | aAC/aGT | N/S | Unchanged |
| PAX7 | 1-19027238-CA-TG | cCA/cTG | P/L | Unchanged |
| UBR4 | 1-19433449-AAC-GAT | GtT/AtC | V/I | Unchanged |
| EMC1 | 1-19566381-CA-TG | tTG/tCA | L/S | Unchanged |
| AKR7A3 | 1-19612476-AC-GT | GTg/ACg | V/T | Changed missense |
| TMCO4 | 1-20020993-AC-GT | cGT/cAC | R/H | Unchanged |
| EPHA8 | 1-22919903-AC-GT | aAC/aGT | N/S | Unchanged |
| STPG1 | 1-24710404-CA-TG | aTG/aCA | M/T | Partially changed missense |
| LDLRAP1 | 1-25890188-CA-TG | aCA/aTG | T/M | Changed missense |
| AC114494.1 | 1-31902417-CA-TG | aTG/aCA | M/T | Partially changed missense |
| HCRTR1 | 1-32092525-ATC-GTT | AtC/GtT | I/V | Unchanged |
| ZSCAN20 | 1-33960061-CA-TG | gCA/gTG | A/V | Unchanged |
| AGO1 | 1-36359668-CA-TG | cCA/cTG | P/L | Unchanged |
| TEKT2 | 1-36553672-CA-TG | aCA/aTG | T/M | Changed missense |
| CSF3R | 1-36937065-AC-GT | cGT/cAC | R/H | Unchanged |
| GRIK3 | 1-37267555-AC-GT | GTc/ACc | V/T | Changed missense |
| DNALI1 | 1-38023316-CA-TG | gCA/gTG | A/V | Unchanged |
| INPP5B | 1-38338794-CA-TG | aTG/aCA | M/T | Partially changed missense |
| NFYC | 1-41204567-AGC-GGT | AgC/GgT | S/G | Unchanged |
| HIVEP3 | 1-42046527-CA-GT | aTG/aAC | M/N | Changed missense |
| GUCA2B | 1-42619138-CA-TG | gCA/gTG | A/V | Unchanged |
| FOXJ3 | 1-42693596-CA-TG | gTG/gCA | V/A | Unchanged |
| TIE1 | 1-43784954-CTA-TTG | CtA/TtG | L | Unchanged |
| AKR1A1 | 1-46027527-AC-GT | ACc/GTc | T/V | Changed missense |
| MAST2 | 1-46500882-AC-GT | aAC/aGT | N/S | Unchanged |
| SLC1A7 | 1-53558270-AC-GT | cGT/cAC | R/H | Unchanged |
| MROH7 | 1-55119143-CA-TG | CAt/TGt | H/C | Changed missense |
| MROH7-TTC4 | 1-55119143-CA-TG | CAt/TGt | H/C | Changed missense |
| MROH7-TTC4 | 1-55139740-CA-TG | CAc/TGc | H/C | Changed missense |
| MROH7 | 1-55139740-CA-TG | CAc/TGc | H/C | Changed missense |
| KANK4 | 1-62728783-CA-TG | gTG/gCA | V/A | Unchanged |
| KANK4 | 1-62728784-AC-GT | GTg/ACg | V/T | Changed missense |
| LEPR | 1-66058512-CA-TG | CAg/TGg | Q/W | Rescued nonsense |
| AK5 | 1-77759577-CA-TG | gCA/gTG | A/V | Unchanged |
| IFI44L | 1-79093817-CA-TG | CAt/TGt | H/C | Changed missense |
| SSX2IP | 1-85113228-CA-TG | TGt/CAt | C/H | Changed missense |
| CLCA2 | 1-86920902-CAA-TAG | CaA/TaG | Q/* | Unchanged |
| CDC7 | 1-91980446-CA-TG | aCA/aTG | T/M | Changed missense |
| ARHGAP29 | 1-94639446-AC-GT | gGT/gAC | G/D | Unchanged |
| AMY2A | 1-104162194-AC-GT | ACt/GTt | T/V | Changed missense |
| KIAA1324 | 1-109737062-AC-GT | aAC/aGT | N/S | Unchanged |
| OVGP1 | 1-111957692-CA-TG | aTG/aCA | M/T | Partially changed missense |
| ATP5F1 | 1-112002206-AC-GT | cAC/cGT | H/R | Unchanged |
| PPM1J | 1-113255388-AC-GT | GTt/ACt | V/T | Changed missense |
| PTPN22 | 1-114377567-CA-TG | TGg/CAg | W/Q | Rescued nonsense |
| IGSF3 | 1-117142868-CA-TG | TGg/CAg | W/Q | Rescued nonsense |
| NBPF16 | 1-148756446-AC-GT | aAC/aGT | N/S | Unchanged |
| CGN | 1-151502977-CA-TG | CAg/TGg | Q/W | Rescued nonsense |
| SNX27 | 1-151634729-AC-GT | ACa/GTa | T/V | Changed missense |
| THEM5 | 1-151825919-CA-TG | aTG/aCA | M/T | Partially changed missense |
| HRNR | 1-152190349-CA-TG | tTG/tCA | L/S | Unchanged |
| FLG | 1-152277363-CCA-TCG | TgG/CgA | W/R | Rescued nonsense |
| FLG | 1-152277382-AC-TG | GTc/CAc | V/H | Changed missense |
| FLG | 1-152277402-AC-GT | cGT/cAC | R/H | Unchanged |
| FLG | 1-152279715-CA-TG | gTG/gCA | V/A | Unchanged |
| FLG | 1-152279919-CA-TG | tTG/tCA | L/S | Unchanged |
| FLG | 1-152280687-CA-TG | gTG/gCA | V/A | Unchanged |
| FLG | 1-152283333-CA-TG | gTG/gCA | V/A | Unchanged |
| FLG | 1-152284170-CCA-TCG | TgG/CgA | W/R | Rescued nonsense |
| FLG | 1-152284854-ATC-GTT | GaT/AaC | D/N | Unchanged |
| FLG | 1-152285553-CA-TG | gTG/gCA | V/A | Unchanged |
| FLG | 1-152286126-AC-GT | cGT/cAC | R/H | Unchanged |
| FLG | 1-152286342-ATC-TTG | GaT/CaA | D/Q | Changed missense |
| FLG | 1-152286385-CA-TG | TGg/CAg | W/Q | Rescued nonsense |
| LCE2A | 1-152671387-CA-TG | CAg/TGg | Q/W | Rescued nonsense |
| LCE1E | 1-152760074-CA-TG | tCA/tTG | S/L | Unchanged |
| LCE1D | 1-152770506-AC-GT | cAC/cGT | H/R | Unchanged |
| IVL | 1-152882860-CA-TG | cCA/cTG | P/L | Unchanged |
| LOR | 1-153233510-AGC-GGT | AgC/GgT | S/G | Unchanged |
| ATP8B2 | 1-154320941-CA-TG | aCA/aTG | T/M | Changed missense |
| ZBTB7B | 1-154988956-CA-TG | cCA/cTG | P/L | Unchanged |
| HCN3 | 1-155247394-CA-GT | CAg/GTg | Q/V | Changed missense |
| GON4L | 1-155735584-AC-TG | GTg/CAg | V/Q | Changed missense |
| GON4L | 1-155736381-CA-TG | tTG/tCA | L/S | Unchanged |
| APOA1BP | 1-156563850-ACC-GCT | AcC/GcT | T/A | Unchanged |
| FCRL5 | 1-157509024-AC-GT | gGT/gAC | G/D | Unchanged |
| CD1E | 1-158324424-CA-TG | CAg/TGg | Q/W | Rescued nonsense |
| OR6P1 | 1-158533224-AC-GT | cGT/cAC | R/H | Unchanged |
| MNDA | 1-158813818-CA-TG | cCA/cTG | P/L | Unchanged |
| ATP1A4 | 1-160141195-AC-GT | tAC/tGT | Y/C | Unchanged |
| COPA | 1-160281738-CA-GT | aTG/aAC | M/N | Changed missense |
| ADAMTS4 | 1-161168004-CGA-TGG | TcG/CcA | S/P | Unchanged |
| FCGR2A | 1-161476204-CA-TG | CAg/TGg | Q/W | Rescued nonsense |
| ATF6 | 1-161928326-CA-TG | tCA/tTG | S/L | Unchanged |
| POGK | 1-166818565-CA-TG | gCA/gTG | A/V | Unchanged |
| ADCY10 | 1-167849414-ACC-GCT | GgT/AgC | G/S | Unchanged |
| XCL1 | 1-168550337-CA-TG | aCA/aTG | T/M | Changed missense |
| FMO2 | 1-171168584-CA-TG | tCA/tTG | S/L | Unchanged |
| SUCO | 1-172558523-CA-TG | cCA/cTG | P/L | Unchanged |
| ZBTB37 | 1-173855207-CA-TG | aCA/aTG | T/M | Changed missense |
| TNR | 1-175324651-ATC-GTT | GaT/AaC | D/N | Unchanged |
| QSOX1 | 1-180145087-CA-TG | aCA/aTG | T/M | Changed missense |
| KIAA1614 | 1-180913597-AAC-GAT | AaC/GaT | N/D | Unchanged |
| IER5 | 1-181058642-CA-TG | CAa/TGa | Q/* | Unchanged |
| RGSL1 | 1-182499956-CA-TG | CAc/TGc | H/C | Changed missense |
| SMG7 | 1-183510142-AC-GT | cAC/cGT | H/R | Unchanged |
| FAM129A | 1-184765071-CA-TG | cTG/cCA | L/P | Unchanged |
| SWT1 | 1-185171868-CA-TG | CAc/TGc | H/C | Changed missense |
| HMCN1 | 1-186101538-CA-TG | CAa/TGa | Q/* | Unchanged |
| CFHR1 | 1-196797356-CA-TG | aCA/aTG | T/M | Changed missense |
| IGFN1 | 1-201175273-CA-TG | CAg/TGg | Q/W | Rescued nonsense |
| ATP2B4 | 1-203652443-CA-TG | tCA/tTG | S/L | Unchanged |
| DSTYK | 1-205130412-CA-TG | TGt/CAt | C/H | Changed missense |
| CR1 | 1-207753620-CA-TG | CAt/TGt | H/C | Changed missense |
| CR1L | 1-207850878-AC-GT | aAC/aGT | N/S | Unchanged |
| CAMK1G | 1-209782342-CA-TG | cCA/cTG | P/L | Unchanged |
| TRAF5 | 1-211533351-CA-TG | gCA/gTG | A/V | Unchanged |
| FAM71A | 1-212799870-AAC-GAT | AaC/GaT | N/D | Unchanged |
| 43891 | 1-220970028-ACC-GCT | AcC/GcT | T/A | Unchanged |
| HHIPL2 | 1-222721288-CA-TG | tTG/tCA | L/S | Unchanged |
| EPHX1 | 1-226026405-CA-TG | CAt/TGt | H/C | Changed missense |
| PARP1 | 1-226555301-CA-TG | gTG/gCA | V/A | Unchanged |
| OBSCN | 1-228402120-CA-TG | CAg/TGg | Q/W | Rescued nonsense |
| OBSCN | 1-228431094-CA-TG | aCA/aTG | T/M | Changed missense |
| OBSCN | 1-228433216-CA-TG | aCA/aTG | T/M | Changed missense |
| OBSCN | 1-228461128-CA-TG | CAc/TGc | H/C | Changed missense |
| OBSCN | 1-228465346-AAC-GAT | AaC/GaT | N/D | Unchanged |
| OBSCN | 1-228480281-CA-TG | aCA/aTG | T/M | Changed missense |
| OBSCN | 1-228503676-CA-TG | CAc/TGc | H/C | Changed missense |
| OBSCN | 1-228503677-AC-GT | cAC/cGT | H/R | Unchanged |
| OBSCN | 1-228560138-CA-TG | tCA/tTG | S/L | Unchanged |
| AGT | 1-230845793-CA-TG | aTG/aCA | M/T | Partially changed missense |
| CAPN9 | 1-230898493-AC-GT | aAC/aGT | N/S | Unchanged |
| SPRTN | 1-231487306-CA-TG | gCA/gTG | A/V | Unchanged |
| NID1 | 1-236208772-AC-GT | GTt/ACt | V/T | Changed missense |
| HEATR1 | 1 -236716959-CGA-TGG | TcG/CcA | S/P | Unchanged |
| HEATR1 | 1-236719192-CA-TG | gTG/gCA | V/A | Unchanged |
| MTR | 1-237Q54568-CA-TG | gCA/gTG | A/V | Unchanged |
| KIF26B | 1-245851303-CA-TG | aCA/aTG | T/M | Changed missense |
| OR2T8 | 1-248084470-CA-TG | CAc/TGc | H/C | Changed missense |
| OR2L8 | 1-248112835-CA-TG | CAc/TGc | H/C | Changed missense |
| OR2L3 | 1-248224587-ACC-GCT | AcC/GcT | T/A | Unchanged |
| OR2M3 | 1-248366682-AC-GT | ACa/GTa | T/V | Changed missense |
| OR2T33 | 1-248436616-AC-GT | tGT/tAC | C/Y | Unchanged |
| OR2T12 | 1-248458735-CA-TG | TGg/CAg | W/Q | Rescued nonsense |
| OR2T3 | 1-248637064-CA-TG | cCA/cTG | P/L | Unchanged |
| OR2T34 | 1-248737453-CA-TG | aTG/aCA | M/T | Partially changed missense |
| OR2T11 | 1-248790074-CA-TG | TGc/CAc | C/H | Changed missense |
| OR14I1 | 1-248845096-AC-GT | aGT/aAC | S/N | Unchanged |
| OR14I1 | 1-248845498-CA-TG | gTG/gCA | V/A | Unchanged |
| PFKP | 10-3155619-CA-TG | gCA/gTG | A/V | Unchanged |
| PITRM1 | 10-3181125-AC-GT | GTa/ACa | V/T | Changed missense |
| AKR1E2 | 10-4872910-AC-GT | gAC/gGT | D/G | Unchanged |
| AKR1C1 | 10-5010571-CA-TG | aCA/aTG | T/M | Changed missense |
| AKR1C4 | 10-5255024-CA-TG | CAa/TGa | Q/* | Unchanged |
| NET1 | 10-5498692-CA-TG | cCA/cTG | P/L | Unchanged |
| ECHDC3 | 10-11805353-CA-TG | gCA/gTG | A/V | Unchanged |
| ITGA8 | 10-15573050-AC-GT | GTa/ACa | V/T | Changed missense |
| PTER | 10-16547056-CAA-TAG | CaA/TaG | Q/* | Unchanged |
| CUBN | 10-16918972-AGC-GGT | GcT/AcC | A/T | Unchanged |
| NEBL | 10-21106620-AC-GT | GTa/ACa | V/T | Changed missense |
| EBLN1 | 10-22498208-CA-TG | aTG/aCA | M/T | Partially changed missense |
| ARMC3 | 10-23297299-AAC-GAT | AaC/GaT | N/D | Unchanged |
| ARMC3 | 10-23297300-AC-GT | aAC/aGT | N/S | Unchanged |
| KIAA1217 | 10-24762464-CA-TG | gCA/gTG | A/V | Unchanged |
| KIAA1217 | 10-24813326-CA-TG | aCA/aTG | T/M | Changed missense |
| GPR158 | 10-25888180-ATC-GTT | AtC/GtT | I/V | Unchanged |
| NRP1 | 10-33552695-CA-TG | gTG/gCA | V/A | Unchanged |
| RET | 10-43595967-CA-TG | gCA/gTG | A/V | Unchanged |
| RET | 10-43606686-CA-TG | gCA/gTG | A/V | Unchanged |
| GPRIN2 | 10-47000187-CA-TG | cCA/cTG | P/L | Unchanged |
| ASAH2C | 10-48003991-AC-GT | tGT/tAC | C/Y | Unchanged |
| ZWINT | 10-58119595-AGC-GGT | GcT/AcC | A/T | Unchanged |
| TMEM26 | 10-63170290-CAA-TAG | TtG/CtA | L | Unchanged |
| ADAMTS14 | 10-72520329-CA-TG | gCA/gTG | A/V | Unchanged |
| UNC5B | 10-73046617-ATC-GTT | AtC/GtT | I/V | Unchanged |
| UNC5B | 10-73056495-CA-TG | gCA/gTG | A/V | Unchanged |
| RPS24 | 10-79814598-CA-TG | CAc/TGc | H/C | Changed missense |
| SFTPA1 | 10-81372080-CA-TG | cCA/cTG | P/L | Unchanged |
| NUTM2E | 10-81608367-CA-TG | CAg/TGg | Q/W | Rescued nonsense |
| SFTPD | 10-81706323-CA-TG | aTG/aCA | M/T | Partially changed missense |
| CDHR1 | 10-85960394-CA-TG | gCA/gTG | A/V | Unchanged |
| CEP55 | 10-95262981-AC-GT | ACa/GTa | T/V | Changed missense |
| PLCE1 | 10-96066341-AC-GT | cAC/cGT | H/R | Unchanged |
| R3HCC1L | 10-99969567-CA-TG | CAc/TGc | H/C | Changed missense |
| PYROXD2 | 10-100148175-CA-TG | aTG/aCA | M/T | Partially changed missense |
| HPS1 | 10-100202987-AC-GT | GTc/ACc | V/T | Changed missense |
| CWF19L1 | 10-102016043-AC-GT | tGT/tAC | C/Y | Unchanged |
| LZTS2 | 10-102766573-CA-TG | gCA/gTG | A/V | Unchanged |
| NRAP | 10-115352004-CA-TG | cTG/cCA | L/P | Unchanged |
| NRAP | 10-115411613-CA-TG | gTG/gCA | V/A | Unchanged |
| NRAP | 10-115411614-AC-GT | GTg/ACg | V/T | Changed missense |
| ATRNL1 | 10-117228793-CA-TG | aCA/aTG | T/M | Changed missense |
| WDR11 | 10-122649481-CA-TG | gCA/gTG | A/V | Unchanged |
| DMBT1 | 10-124348621-AC-GT | ACg/GTg | T/V | Changed missense |
| DMBT1 | 10-124358455-CA-TG | tCA/tTG | S/L | Unchanged |
| DMBT1 | 10-124358583-CA-TG | CAt/TGt | H/C | Changed missense |
| DMBT1 | 10-124402676-CA-TG | cCA/cTG | P/L | Unchanged |
| LHPP | 10-126172862-CA-TG | CAa/TGa | Q/* | Unchanged |
| CTBP2 | 10-126715153-CA-TG | cTG/cCA | L/P | Unchanged |
| DOCK1 | 10-128926011-CGA-TGG | CgA/TgG | R/W | Rescued nonsense |
| AL450307.1 | 10-133608209-CA-TG | TGc/CAc | C/H | Changed missense |
| JAKMIP3 | 10-133961493-AC-GT | cAC/cGT | H/R | Unchanged |
| STK32C | 10-134036193-AC-GT | cGT/cAC | R/H | Unchanged |
| LRRC27 | 10-134161631-CCA-TCG | CcA/TcG | P/S | Unchanged |
| LRRC27 | 10-134161632-CA-TG | cCA/cTG | P/L | Unchanged |
| PRAP1 | 10-135165789-CA-TG | CAt/TGt | H/C | Changed missense |
| SPRN | 10-135237060-AC-GT | gGT/gAC | G/D | Unchanged |
| CYP2E1 | 10-135342049-AC-TG | cAC/cTG | H/L | Partially changed missense |
| SCGB1C1 | 11-193721-CA-TG | aCA/aTG | T/M | Changed missense |
| PSMD13 | 11-237087-AC-GT | aAC/aGT | N/S | Unchanged |
| PSMD13 | 11-244166-AC-GT | aAC/aGT | N/S | Unchanged |
| ATHL1 | 11-290815-CA-TG | gCA/gTG | A/V | Unchanged |
| ANO9 | 11-428384-CA-TG | TGc/CAc | C/H | Changed missense |
| MUC6 | 11-1017755-CA-TG | cTG/cCA | L/P | Unchanged |
| MUC2 | 11-1075678-AC-GT | aAC/aGT | N/S | Unchanged |
| MUC5B | 11-1264361-CA-TG | aCA/aTG | T/M | Changed missense |
| MUC5B | 11-1264631-AC-GT | aAC/aGT | N/S | Unchanged |
| MUC5B | 11-1267619-CA-TG | aCA/aTG | T/M | Changed missense |
| BRSK2 | 11-1481930-ACC-GCT | AcC/GcT | T/A | Unchanged |
| MOB2 | 11-1507730-CA-TG | cTG/cCA | L/P | Unchanged |
| OR51F1 | 11-4790671-CA-TG | tTG/LCA | L/S | Unchanged |
| OR51B2 | 11-5345169-CA-TG | TGt/CAt | C/H | Changed missense |
| OR51B5 | 11-5364692-AGC-GGT | GcT/AcC | A/T | Unchanged |
| OR52D1 | 11-5510283-CA-TG | tCA/tTG | S/L | Unchanged |
| OR52H1 | 11-5566029-CA-TG | TGc/CAc | C/H | Changed missense |
| OR52B6 | 11-5602614-CA-TG | CAc/TGc | H/C | Changed missense |
| OR52B6 | 11-5602615-AC-GT | cAC/cGT | H/R | Unchanged |
| OR52L1 | 11-6007271-CA-TG | TGg/CAg | W/Q | Rescued nonsense |
| OR52L1 | 11-6007679-CA-TG | TGc/CAc | C/H | Changed missense |
| OR52W1 | 11-6221168-CA-TG | CAt/TGt | H/C | Changed missense |
| DNHD1 | 11-6550003-CA-TG | CAg/TGg | Q/W | Rescued nonsense |
| OR2AG2 | 11-6789256-CA-TG | cTG/cCA | L/P | Unchanged |
| OR2AG1 | 11-6807200-CA-TG | cCA/cTG | P/L | Unchanged |
| ZNF214 | 11-7022159-CA-TG | TGt/CAt | C/H | Changed missense |
| OR10A6 | 11-7949349-CA-TG | cTG/cCA | L/P | Unchanged |
| TUB | 11-8122089-AGC-GGT | AgC/GgT | S/G | Unchanged |
| SWAP70 | 11-9754220-AC-GT | aAC/aGT | N/S | Unchanged |
| MRGPRX3 | 11-18159119-ATC-GTT | AtC/GtT | I/V | Unchanged |
| MRGPRX3 | 11-18159168-CA-TG | tCA/tTG | S/L | Unchanged |
| MRGPRX4 | 11-18194931-CA-TG | aCA/aTG | T/M | Changed missense |
| MRGPRX4 | 11-18195722-CA-TG | CAg/TGg | Q/W | Rescued nonsense |
| SAA4 | 11-18253176-CA-TG | TGc/CAc | C/H | Changed missense |
| SAA2 | 11-18267026-AC-GT | cGT/cAC | R/H | Unchanged |
| IGSF22 | 11-18738280-AC-GT | GTt/ACt | V/T | Changed missense |
| SLC6A5 | 11-20639399-CA-TG | tCA/tTG | S/L | Unchanged |
| SLC5A12 | 11-26702721-CA-TG | gTG/gCA | V/A | Unchanged |
| NAT10 | 11-34130341-CA-TG | tCA/tTG | S/L | Unchanged |
| LDLRAD3 | 11-36103227-CA-TG | cCA/cTG | P/L | Unchanged |
| RAG1 | 11-36597733-CA-TG | gCA/gTG | A/V | Unchanged |
| C11orf96 | 11-43946967-AC-GT | ACg/GTg | T/V | Changed missense |
| DGKZ | 11-46387867-CA-TG | CAg/TGg | Q/W | Rescued nonsense |
| DGKZ | 11-46397675-CA-GT | CAc/GTc | H/V | Changed missense |
| OR4S1 | 11-48328642-AAC-GAT | AaC/GaT | N/D | Unchanged |
| TRIM64C | 11-49080503-CA-TG | tTG/tCA | L/S | Unchanged |
| OR4C46 | 11-51515639-CA-TG | CAc/TGc | H/C | Changed missense |
| OR4C16 | 11-55339829-AC-GT | ACc/GTg | T/V | Changed missense |
| OR4C16 | 11-55339829-ACC-GCT | AcC/GcT | T/A | Unchanged |
| OR5L1 | 11-55579340-CA-TG | aCA/aTG | T/M | Changed missense |
| OR5F1 | 11-55761220-AC-GT | aGT/aAC | S/N | Unchanged |
| OR5J2 | 11-55944778-CA-TG | CAc/TGc | H/C | Changed missense |
| OR8U1 | 11-56143157-CA-TG | CAt/TGt | H/C | Changed missense |
| OR8U1 | 11-56143976-CA-TG | CAg/TGg | Q/W | Rescued nonsense |
| OR5R1 | 11-56185344-CA-TG | TGc/CAc | C/H | Changed missense |
| OR1S1 | 11-57982619-CA-TG | CAc/TGc | H/C | Changed missense |
| OR5B12 | 11-58207203-CA-TG | TGc/CAc | C/H | Changed missense |
| FAM111A | 11-58919812-CA-TG | gCA/gTG | A/V | Unchanged |
| OR4D10 | 11-59245494-CTA-TTG | CtA/TtG | L | Unchanged |
| MS4A4A | 11-60059738-CA-TG | CAg/TGg | Q/W | Rescued nonsense |
| CD5 | 11-60892605-CA-TG | CAc/TGc | H/C | Changed missense |
| SLC22A24 | 11-62848568-CA-GT | gTG/gAC | V/D | Changed missense |
| NUDT22 | 11-63997328-CA-TG | CAg/TGg | Q/W | Rescued nonsense |
| PLCB3 | 11-64022458-CA-TG | cCA/cTG | P/L | Unchanged |
| SLC22A12 | 11-64367324-CA-TG | gCA/gTG | A/V | Unchanged |
| TMEM262 | 11-64856498-CCA-TCG | TgG/CgA | W/R | Rescued nonsense |
| TMEM262 | 11-64856499-CA-TG | TGg/CAg | W/Q | Rescued nonsense |
| PCNXL3 | 11-65385605-CA-TG | CAg/TGg | Q/W | Rescued nonsense |
| SPTBN2 | 11-66468468-CA-TG | gTG/gCA | V/A | Unchanged |
| TBC1D10C | 11-67176897-CA-TG | tCA/tTG | S/L | Unchanged |
| GPR152 | 11-67218909-AGC-GGT | GcT/AcC | A/T | Unchanged |
| AIP | 11-67258390-CA-TG | CAg/TGg | Q/W | Rescued nonsense |
| ALDH3B2 | 11-67434160-CA-TG | aTG/aCA | M/T | Partially changed missense |
| ALDH3B1 | 11-67789137-AC-GT | tAC/tGT | Y/C | Unchanged |
| IGHMBP2 | 11-68703959-AC-GT | ACg/GTg | T/V | Changed missense |
| ANO1 | 11-70007391-CA-TG | gCA/gTG | A/V | Unchanged |
| PPFIA1 | 11-70221159-CA-TG | gCA/gTG | A/V | Unchanged |
| DHCR7 | 11-71146691-ATC-GTT | GaT/AaC | D/N | Unchanged |
| DEFB1Q8B | 11-71548543-CA-TG | CAa/TGa | Q/* | Unchanged |
| FOLR1 | 11-71907094-CA-TG | cCA/cTG | P/L | Unchanged |
| PDE2A | 11-72293602-ATC-GTT | GaT/AaC | D/N | Unchanged |
| PLEKHB1 | 11-73371892-AC-GT | tAC/tGT | Y/C | Unchanged |
| XRRA1 | 11-74554492-AC-GT | GTc/ACc | V/T | Changed missense |
| FAT3 | 11-92531355-CA-TG | CAg/TGg | Q/W | Rescued nonsense |
| KIAA1731 | 11-93402005-CA-TG | gCA/gTG | A/V | Unchanged |
| PIWIL4 | 11-94318670-CA-TG | cCA/cTG | P/L | Unchanged |
| KDM4E | 11-94759058-CA-TG | CAg/TGg | Q/W | Rescued nonsense |
| ARHGAP42 | 11-100846935-CA-TG | cCA/cTG | P/L | Unchanged |
| ARHGAP42 | 11-100847049-CA-TG | tCA/tTG | S/L | Unchanged |
| DYNC2H1 | 11-103057046-CGA-TGG | CgA/TgG | R/W | Rescued nonsense |
| EXPH5 | 11-108383675-CA-TG | cTG/cCA | L/P | Unchanged |
| ZC3H12C | 11-110035260-CGA-TGG | CgA/TgG | R/W | Rescued nonsense |
| LAYN | 11-111420363-CA-TG | cCA/cTG | P/L | Unchanged |
| BUD13 | 11-116627892-CA-TG | TGg/CAg | W/Q | Rescued nonsense |
| BACE1 | 11-117160346-CA-TG | TGc/CAc | C/H | Changed missense |
| CEP164 | 11-117252437-AC-GT | cAC/cGT | H/R | Unchanged |
| CEP164 | 11-117267883-CA-TG | CAg/TGg | Q/W | Rescued nonsense |
| IL10RA | 11-117864046-CA-TG | gCA/gTG | A/V | Unchanged |
| AMICA1 | 11-118074337-AC-GT | GTg/ACg | V/T | Changed missense |
| TMEM25 | 11-118405067-CA-TG | CAa/TGa | Q/* | Unchanged |
| BCL9L | 11-118771746-CA-TG | gTG/gCA | V/A | Unchanged |
| VPS11 | 11-118947633-AC-GT | tAC/tGT | Y/C | Unchanged |
| HINFP | 11-119005086-ATC-GTT | AtC/GtT | I/V | Unchanged |
| TRIM29 | 11-119999189-CAA-TAG | TtG/CtA | L | Unchanged |
| OAF | 11-120099658-AC-GT | cAC/cGT | H/R | Unchanged |
| POU2F3 | 11-120175748-CA-TG | CAc/TGc | H/C | Changed missense |
| TECTA | 11-120996291-CA-TG | gCA/gTG | A/V | Unchanged |
| UBASH3B | 11-122665436-CA-TG | tCA/tTG | S/L | Unchanged |
| ZNF202 | 11-123600474-CA-TG | gTG/gCA | V/A | Unchanged |
| OR6T1 | 11-123814489-AC-GT | aGT/aAG | S/N | Unchanged |
| OR10G4 | 11-123886319-CA-TG | aCA/aTG | T/M | Changed missense |
| OR10G4 | 11-123886795-CA-TG | CAg/TGg | Q/W | Rescued nonsense |
| OR10D3 | 11-124056581-AC-GT | aAC/aGT | N/S | Unchanged |
| OR8B4 | 11-124294235-CA-TG | TGt/CAt | C/H | Changed missense |
| ST14 | 11-130066334-AC-GT | aAC/aGT | N/S | Unchanged |
| WNK1 | 12-939301-CA-TG | gCA/gTG | A/V | Unchanged |
| TULP3 | 12-3046801-CA-TG | aCA/aTG | T/M | Changed missense |
| ANO2 | 12-6030404-CA-TG | gTG/gCA | V/A | Unchanged |
| CD163L1 | 12-7527123-CA-TG | aTG/aCA | M/T | Partially changed missense |
| A2ML1 | 12-9016572-CA-TG | CAc/TGc | H/C | Changed missense |
| PHC1 | 12-9086898-AC-GT | ACt/GTt | T/V | Changed missense |
| CLEC12B | 12-10165468-CA-TG | aCA/aTG | T/M | Changed missense |
| KLRC3 | 12-10570965-CA-GT | TGt/ACt | C/T | Changed missense |
| NKG2-E | 12-10570965-CA-GT | TGt/ACt | C/T | Changed missense |
| KLRC3 | 12-10573093-CCA-TCG | TgG/CgA | W/R | Rescued nonsense |
| TAS2R9 | 12-10962114-CA-TG | gTG/gCA | V/A | Unchanged |
| PLBD1 | 12-14688643-AC-GT | GTc/ACc | V/T | Changed missense |
| SMCO3 | 12-14959469-CA-TG | TGc/CAc | C/H | Changed missense |
| SMCO2 | 12-27648785-CA-TG | gCA/gTG | A/V | Unchanged |
| VDR | 12-48272894-CA-TG | aTG/aCA | M/T | Partially changed missense |
| PFKM | 12-48527184-ACC-GCT | AcC/GcT | T/A | Unchanged |
| OR10AD1 | 12-48597052-CA-TG | gTG/gCA | V/A | Unchanged |
| CCDC65 | 12-49308283-CA-TG | CAc/TGc | H/C | Changed missense |
| CERS5 | 12-50537814-CA-TG | TGt/CAt | C/H | Changed missense |
| ANKRD33 | 12-52284499-CA-TG | CAg/TGg | Q/W | Rescued nonsense |
| KRT7 | 12-52631312-CA-TG | CAc/TGc | H/C | Changed missense |
| KRT84 | 12-52774234-CA-TG | TGc/CAc | C/H | Changed missense |
| KRT6C | 12-52863212-AC-GT | GTt/ACt | V/T | Changed missense |
| KRT79 | 12-53227802-CA-TG | tTG/tCA | L/S | Unchanged |
| C12orf10 | 12-53700847-AC-GT | ACt/GTt | T/V | Changed missense |
| C12orf10 | 12-53700859-CA-TG | CAc/TGc | H/C | Changed missense |
| OR10P1 | 12-5603161O-CA-TG | tCA/tTG | S/L | Unchanged |
| NACA | 12-57109791-CA-TG | cTG/cCA | L/P | Unchanged |
| SDR9C7 | 12-57324078-AC-GT | cGT/cAC | R/H | Unchanged |
| ARHGEF25 | 12-58010162-CA-TG | CAg/TGg | Q/W | Rescued nonsense |
| PTPRB | 12-70928724-ATC-GTT | GaT/AaC | D/N | Unchanged |
| TMEM19 | 12-72094692-AAC-GAT | AaC/GaT | N/D | Unchanged |
| TRHDE | 12-73014916-CA-TG | aCA/aTG | T/M | Changed missense |
| SYT1 | 12-79611373-AC-GT | aAC/aGT | N/S | Unchanged |
| METTL25 | 12-82792941-CA-TG | cCA/cTG | P/L | Unchanged |
| PLXNC1 | 12-94645254-CA-TG | aCA/aTG | T/M | Changed missense |
| CCDC41 | 12-94769758-AC-GT | cGT/cAC | R/H | Unchanged |
| C12orf55 | 12-97073470-CA-TG | CAt/TGt | H/C | Changed missense |
| MYBPC1 | 12-102069076-CA-TG | cCA/cTG | P/L | Unchanged |
| MYO1H | 12-109853348-CA-TG | cCA/cTG | P/L | Unchanged |
| IFT81 | 12-110618233-CGA-TGG | CgA/TgG | R/W | Rescued nonsense |
| HVCN1 | 12-111099002-CA-TG | aTG/aCA | M/T | Partially changed missense |
| DTX1 | 12-113532615-CGA-TGG | CgA/TgG | R/W | Rescued nonsense |
| TMEM233 | 12-120067643-ATC-GTT | AtC/GtT | I/V | Unchanged |
| COQ5 | 12-120966921-AGC-GGT | GcT/AcC | A/T | Unchanged |
| HIP1R | 12-123333064-AC-GT | tAC/tGT | Y/C | Unchanged |
| HIP1R | 12-123334441-AC-GT | tAC/tGT | Y/C | Unchanged |
| GTF2H3 | 12-124144394-CA-TG | cCA/cTG | P/L | Unchanged |
| DNAH10 | 12-124387585-CA-TG | gCA/gTG | A/V | Unchanged |
| SCARB1 | 12-125284748-AGC-GGT | GcT/AcC | A/T | Unchanged |
| TMEM132C | 12-128899672-CA-TG | CAc/TGc | H/C | Changed missense |
| TMEM132C | 12-128899673-AC-GT | cAC/cGT | H/R | Unchanged |
| TMEM132C | 12-129181809-CA-TG | aCA/aTG | T/M | Changed missense |
| GPR133 | 12-131620587-CA-TG | aCA/aTG | T/M | Changed missense |
| EP400 | 12-132528770-AC-GT | ACc/GTc | T/V | Changed missense |
| EP400 | 12-132528770-ACC-GCT | AcC/GcT | T/A | Unchanged |
| EP400 | 12-132549300-AC-GT | ACa/GTa | T/V | Changed missense |
| FGF9 | 13-22275393-CA-TG | tCA/tTG | S/L | Unchanged |
| TNFRSF19 | 13-24234516-CA-TG | tCA/tTG | S/L | Unchanged |
| C1QTNF9B | 13-24465716-CA-TG | gTG/gCA | V/A | Unchanged |
| C1QTNF9B | 13-24468328-CA-TG | TGt/CAt | C/H | Changed missense |
| PARP4 | 13-25008629-CA-TG | cTG/cCA | L/P | Unchanged |
| PARP4 | 13-25027743-CA-TG | aTG/aCA | M/T | Partially changed missense |
| ATP12A | 13-25265102-CA-TG | gCA/gTG | A/V | Unchanged |
| RNF17 | 13-25419175-CA-TG | aCA/aTG | T/M | Changed missense |
| SLC46A3 | 13-29287551-AC-GT | GTt/ACt | V/T | Changed missense |
| FRY | 13-32676113-CA-TG | aCA/aTG | T/M | Changed missense |
| BRCA2 | 13-32913053-CTA-TTG | CtA/TtG | L | Unchanged |
| POSTN | 13-38156519-AC-GT | GTg/ACc | V/T | Changed missense |
| PROSER1 | 13-39587676-CA-TG | gTG/gCA | V/A | Unchanged |
| MRPS31 | 13-41323395-ATC-GTT | GaT/AaC | D/N | Unchanged |
| TSC22D1 | 13-45148904-AC-GT | GTa/ACa | V/T | Changed missense |
| LRCH1 | 13-47308108-CA-TG | gCA/gTG | A/V | Unchanged |
| PHF11 | 13-50080845-CTA-TTG | CtA/TtG | L | Unchanged |
| ARL11 | 13-50205139-CA-TG | CAt/TGt | H/C | Changed missense |
| PCCA | 13-100888121-CA-TG | gCA/gTG | A/V | Unchanged |
| PCCA | 13-100955203-CA-TG | cCA/cTG | P/L | Unchanged |
| TMTC4 | 13-101287339-AC-GT | GTt/ACt | V/T | Changed missense |
| SLC10A2 | 13-103718468-CA-TG | tTG/tCA | L/S | Unchanged |
| PROZ | 13-113818899-CA-TG | aCA/aTG | T/M | Changed missense |
| CDC16 | 13-115016035-CA-TG | aCA/aTG | T/M | Changed missense |
| POTEG | 14-19566035-AC-GT | tAC/tGT | Y/C | Unchanged |
| POTEM | 14-20019783-AGC-GGT | GcT/AcC | A/T | Unchanged |
| OR4Q3 | 14-20215989-AC-GT | ACa/GTa | T/V | Changed missense |
| OR4L1 | 14-20528361-CA-TG | tCA/tTG | S/L | Unchanged |
| RPGRIP1 | 14-21770680-CA-TG | cCA/cTG | P/L | Unchanged |
| CHD8 | 14-21871653-CA-TG | gTG/gCA | V/A | Unchanged |
| OR4E2 | 14-22133996-CA-TG | CAg/TGg | Q/W | Rescued nonsense |
| TRAV27 | 14-22616546-CA-TG | gCA/gTG | A/V | Unchanged |
| TRAV36DV7 | 14-22695128-ATC-GTT | AtC/GtT | I/V | Unchanged |
| AE000662.92 | 14-23026575-AAC-GAT | AaC/GaT | N/D | Unchanged |
| OR6J1 | 14-23103354-AC-GT | cGT/cAC | R/H | Unchanged |
| THTPA | 14-24026492-CA-TG | CAc/TGc | H/C | Changed missense |
| AKAP6 | 14-33293021-CA-TG | gCA/gTG | A/V | Unchanged |
| MIA2 | 14-39703323-CA-TG | gCA/gTG | A/V | Unchanged |
| NIN | 14-51211072-AC-GT | tGT/tAC | O/Y | Unchanged |
| FRMD6 | 14-52186971-CA-TG | cCA/cTG | P/L | Unchanged |
| GPR137C | 14-53019944-AGC-GGT | AgC/GgT | S/G | Unchanged |
| BMP4 | 14-54417522-AC-GT | GTg/ACg | V/T | Changed missense |
| DAAM1 | 14-59798152-CTA-TTG | CtA/TtG | L | Unchanged |
| C14orf39 | 14-60923782-AC-GT | GTa/ACa | V/T | Changed missense |
| SYNE2 | 14-64522842-CA-TG | CAc/TGc | H/C | Changed missense |
| PLEKHH1 | 14-68042573-CA-TG | CAc/TGc | H/C | Changed missense |
| SLC8A3 | 14-70634087-AC-GT | cGT/cAC | R/H | Unchanged |
| ADAM21 | 14-70924566-CA-GT | gCA/gGT | A/G | Unchanged |
| DPF3 | 14-73220018-CAA-TAG | TtG/CtA | L | Unchanged |
| PAPLN | 14-73726150-CA-TG | CAc/TGc | H/C | Changed missense |
| ACOT2 | 14-74041747-CA-TG | CAc/TGc | H/C | Changed missense |
| TMEM63C | 14-77697966-CA-TG | gCA/gTG | A/V | Unchanged |
| TMEM63C | 14-77703056-CA-TG | gCA/gTG | A/V | Unchanged |
| RIN3 | 14-93118037-CA-TG | CAt/TGt | H/C | Changed missense |
| BDKRB2 | 14-96707456-CA-TG | aCA/aTG | T/M | Changed missense |
| VRK1 | 14-97299852-CA-TG | gCA/gTG | A/V | Unchanged |
| WDR25 | 14-100847706-CA-TG | CAt/TGt | H/C | Changed missense |
| EXOC3L4 | 14-103576414-CA-TG | CAg/TGg | Q/W | Rescued nonsense |
| KIF26A | 14-104642340-CA-TG | cCA/cTG | P/L | Unchanged |
| KIF26A | 14-104642421-CA-TG | gCA/gTG | A/V | Unchanged |
| KIF26A | 14-104643408-CA-TG | gCA/gTG | A/V | Unchanged |
| KIF26A | 14-104643857-CGA-TGG | CgA/TgG | R/W | Rescued nonsense |
| KIF26A | 14-104646062-AC-TG | ACc/TGc | T/C | Changed missense |
| C14orf180 | 14-105054933-CA-TG | cCA/cTG | P/L | Unchanged |
| INF2 | 14-105180705-CA-TG | cCA/cTG | P/L | Unchanged |
| AHNAK2 | 14-105408726-ATC-TTG | GaT/CaA | D/Q | Changed missense |
| AHNAK2 | 14-105408810-CA-TG | cTG/cCA | L/P | Unchanged |
| AHNAK2 | 14-105410775-ATC-GTT | GaT/AaC | D/N | Unchanged |
| AHNAK2 | 14-105411162-CA-TG | cTG/cCA | L/P | Unchanged |
| AHNAK2 | 14-105411204-AGC-GGT | GcT/AcC | A/T | Unchanged |
| AHNAK2 | 14-105411699-CA-TG | gTG/gCA | V/A | Unchanged |
| AHNAK2 | 14-105414474-CA-TG | aTG/aCA | M/T | Partially changed missense |
| AHNAK2 | 14-105414979-AC-GT | GTg/ACg | V/T | Changed missense |
| AHNAK2 | 14-105415122-CA-TG | cTG/cCA | L/P | Unchanged |
| AHNAK2 | 14-105415473-CA-TG | gTG/gCA | V/A | Unchanged |
| AHNAK2 | 14-105415515-CA-TG | gTG/gCA | V/A | Unchanged |
| AHNAK2 | 14-105415516-AC-GT | GTg/ACg | V/T | Changed missense |
| AHNAK2 | 14-105416454-CA-TG | aTG/aCA | M/T | Partially changed missense |
| AHNAK2 | 14-105416505-CA-TG | aTG/aCA | M/T | Partially changed missense |
| AHNAK2 | 14-105416958-CA-TG | gTG/gCA | V/A | Unchanged |
| AHNAK2 | 14-105416959-AC-GT | GTg/ACg | V/T | Changed missense |
| AHNAK2 | 14-105417144-AGC-GGT | GcT/AcC | A/T | Unchanged |
| AHNAK2 | 14-105417957-ACC-GCT | GgT/AgC | G/S | Unchanged |
| AHNAK2 | 14-105419736-CA-TG | tTG/tCA | L/S | Unchanged |
| IGHV1-2 | 14-106452816-CA-TG | TGg/CAg | W/Q | Rescued nonsense |
| IGHV1-3 | 14-106471267-CA-TG | aTG/aCA | M/T | Partially changed missense |
| IGHV2-5 | 14-106494195-CA-TG | gTG/gCA | V/A | Unchanged |
| IGHV1-8 | 14-106539280-AC-GT | aGT/aAC | S/N | Unchanged |
| IGHV1-8 | 14-106539367-CA-GT | gTG/gAC | V/D | Changed missense |
| IGHV3-9 | 14-106552480-CA-TG | aTG/aCA | M/T | Partially changed missense |
| IGHV1-18 | 14-106641758-AC-TG | gGT/gCA | G/A | Unchanged |
| IGHV4-31 | 14-106805440-AC-GT | tGT/tAC | C/Y | Unchanged |
| IGHV3-43 | 14-106926317-ACC-TCG | GgT/CgA | G/R | Unchanged |
| IGHV3-49 | 14-107013232-CA-GT | gTG/gAC | V/D | Changed missense |
| IGHV3-53 | 14-107049101-CA-TG | TGg/CAg | W/Q | Rescued nonsense |
| IGHV4-61 | 14-107095156-AGC-GGT | GcT/AcC | A/T | Unchanged |
| IGHV4-61 | 14-107095378-CGA-TGG | TcG/CcA | S/P | Unchanged |
| IGHV1-69 | 14-107170219-CA-GT | gTG/gAC | V/D | Changed missense |
| GOLGA8I | 15-23263596-AC-GT | cAC/cGT | H/R | Unchanged |
| RP11-467N20.5 | 15-23406386-CA-TG | TGt/CAt | C/H | Changed missense |
| GOLGA6L2 | 15-23688942-CCA-TCG | TgG/CgA | W/R | Rescued nonsense |
| GOLGA6L2 | 15-23688943-CA-TG | TGg/CAg | W/Q | Rescued nonsense |
| GOLGA8T | 15-30434160-CA-TG | CAg/TGg | Q/W | Rescued nonsense |
| CHRFAM7A | 15-30659682-AC-GT | GTg/ACg | V/T | Changed missense |
| TRPM1 | 15-31369122-CA-TG | aTG/aCA | M/T | Partially changed missense |
| GOLGA8K | 15-32686364-CA-TG | cTG/cCA | L/P | Unchanged |
| FMN1 | 15-33357261-CA-TG | cTG/cCA | L/P | Unchanged |
| RYR3 | 15-34113535-CA-TG | gCA/gTG | A/V | Unchanged |
| THBS1 | 15-39880822-AC-GT | ACa/GTa | T/V | Changed missense |
| FSIP1 | 15-39910430-CA-TG | TGt/CAt | C/H | Changed missense |
| EIF2AK4 | 15-40260064-CAA-GAT | CaA/GaT | Q/D | Changed missense |
| PAK6 | 15-40558481-CA-TG | CAt/TGt | H/C | Changed missense |
| SPINT1 | 15-41137111-AC-GT | aAC/aGT | N/S | Unchanged |
| MGA | 15-42026763-AC-GT | gAC/gGT | D/G | Unchanged |
| STRC | 15-43900291-AC-TG | GTt/CAt | V/H | Changed missense |
| DUOX2 | 15-45388098-CA-TG | gTG/gCA | V/A | Unchanged |
| DUOX1 | 15-45444517-CA-TG | aCA/aTG | T/M | Changed missense |
| MYO5C | 15-52531939-CAA-TAG | TtG/CtA | L | Unchanged |
| GCOM1 | 15-57918068-CA-TG | gCA/gTG | A/V | Unchanged |
| MYZAP | 15-57918068-CA-TG | gCA/gTG | A/V | Unchanged |
| TPM1 | 15-63354415-AC-GT | tAC/tGT | Y/C | Unchanged |
| TRIP4 | 15-64706331-CA-TG | cCA/cTG | P/L | Unchanged |
| PIF1 | 15-65113686-CA-TG | TGt/CAt | C/H | Changed missense |
| IGDCC3 | 15-65667724-AGC-GGT | GcT/AcC | A/T | Unchanged |
| KIF23 | 15-69728947-CCA-TCG | CcA/TcG | P/S | Unchanged |
| KIF23 | 15-69728948-CA-TG | cCA/cTG | P/L | Unchanged |
| SCAMP5 | 15-75305039-CA-TG | cCA/cTG | P/L | Unchanged |
| C15orf39 | 15-75498547-CA-TG | gCA/gTG | A/V | Unchanged |
| TMED3 | 15-79614417-CA-TG | gCA/gTG | A/V | Unchanged |
| IL16 | 15-81592801-CA-TG | aCA/aTG | T/M | Changed missense |
| GOLGA6L10 | 15-82637313-CA-TG | TGt/CAt | C/H | Changed missense |
| GOLGA6L10 | 15-82637397-CA-TG | TGt/CAt | C/H | Changed missense |
| GOLGA6L10 | 15-82637418-CA-TG | TGt/CAt | C/H | Changed missense |
| GOLGA6L18 | 15-82934618-CA-TG | TGt/CAt | C/H | Changed missense |
| GOLGA6L18 | 15-82934660-CA-TG | TGt/CAt | C/H | Changed missense |
| GOLGA6L18 | 15-82934744-CA-TG | TGt/CAt | C/H | Changed missense |
| GOLGA6L18 | 15-82934912-CA-TG | TGt/CAt | C/H | Changed missense |
| GOLGA6L19 | 15-83013600-CA-TG | TGt/CAt | C/H | Changed missense |
| C15orf40 | 15-83680286-CA-TG | TGc/CAc | C/H | Changed missense |
| ZNF592 | 15-85345509-CA-TG | cCA/cTG | P/L | Unchanged |
| SLC28A1 | 15-85478408-CAA-TAG | CaA/TaG | Q/* | Unchanged |
| AGBL1 | 15-86838496-AC-GT | gAC/gGT | D/G | Unchanged |
| AGBL1 | 15-87217612-CA-TG | CAa/TGa | Q/* | Unchanged |
| KIF7 | 15-90174789-CGA-TGG | TcG/CcA | S/P | Unchanged |
| MAN2A2 | 15-91452594-CA-TG | CAg/TGg | Q/W | Rescued nonsense |
| UNC45A | 15-91488184-AC-GT | ACc/GTc | T/V | Changed missense |
| UNC45A | 15-91489918-CA-TG | cCA/cTG | P/L | Unchanged |
| FAM169B | 15-98995079-CGA-TGG | TcG/CcA | S/P | Unchanged |
| SYNM | 15-99671491-CA-TG | CAg/TGg | Q/W | Rescued nonsense |
| ADAMTS17 | 15-100692844-CA-TG | aTG/aCA | M/T | Partially changed missense |
| LRRK1 | 15-101598312-ACC-GCT | AcC/GcT | T/A | Unchanged |
| RGS11 | 16-319510-CA-TG | aTG/aCA | M/T | Partially changed missense |
| NME4 | 16-450239-AC-GT | gAC/gGT | D/G | Unchanged |
| CAPN15 | 16-603470-CA-TG | cCA/cTG | P/L | Unchanged |
| C16orf11 | 16-614819-CGA-TGG | CgA/TgG | R/W | Rescued nonsense |
| PIGQ | 16-624114-AC-GT | ACg/GTg | T/V | Changed missense |
| WDR90 | 16-711904-CA-TG | cCA/cTG | P/L | Unchanged |
| CCDC78 | 16-774691-CA-TG | TGg/CAg | W/Q | Rescued nonsense |
| CHTF18 | 16-842449-AC-GT | aAC/aGT | N/S | Unchanged |
| SSTR5 | 16-1129911-CA-TG | cCA/cTG | P/L | Unchanged |
| CACNA1H | 16-1261218-CA-TG | tCA/tTG | S/L | Unchanged |
| CACNA1H | 16-1261221-CA-TG | tCA/tTG | S/L | Unchanged |
| CCDC154 | 16-1484472-CA-TG | cTG/cCA | L/P | Unchanged |
| CCDC154 | 16-1488646-AC-GT | cGT/cAC | R/H | Unchanged |
| TELO2 | 16-1545447-CA-TG | CAg/TGg | Q/W | Rescued nonsense |
| TELO2 | 16-1555588-CA-TG | CAg/TGg | Q/W | Rescued nonsense |
| ZNF598 | 16-2049002-CA-GT | TGt/ACt | C/T | Changed missense |
| ZNF598 | 16-2049639-CA-TG | aTG/aCA | M/T | Partially changed missense |
| E4F1 | 16-2285355-ATC-GTT | AtC/GtT | I/V | Unchanged |
| ABCA3 | 16-2374466-AC-GT | GTg/ACg | V/T | Changed missense |
| MEFV | 16-3297073-ATC-GTT | GaT/AaC | D/N | Unchanged |
| OR2C1 | 16-3405993-CA-TG | tCA/tTG | S/L | Unchanged |
| VASN | 16-4431201-CA-TG | aCA/aTG | T/M | Changed missense |
| C16orf71 | 16-4796392-CA-TG | CAg/TGg | Q/W | Rescued nonsense |
| ABAT | 16-8839953-CA-TG | CAg/TGg | Q/W | Rescued nonsense |
| CIITA | 16-11002926-CA-TG | CAg/TGg | Q/W | Rescued nonsense |
| CTD-3088G3.8 | 16-11588525-CA-TG | TGc/CAc | C/H | Changed missense |
| TNFRSF17 | 16-12060163-AC-GT | aAC/aGT | N/S | Unchanged |
| SNX29 | 16-12162968-CA-TG | cCA/cTG | P/L | Unchanged |
| NPIPA7 | 16-16481609-CA-TG | aCA/aTG | T/M | Changed missense |
| ACSM5 | 16-20441076-CA-TG | CAc/TGc | H/C | Changed missense |
| ACSM2A | 16-20489898-ATC-GTT | AtC/GtT | I/V | Unchanged |
| EEF2K | 16-22269866-CA-TG | CAa/TGa | Q/* | Unchanged |
| RBBP6 | 16-24583405-CA-TG | gCA/gTG | A/V | Unchanged |
| ZKSCAN2 | 16-25258654-CA-TG | TGg/CAg | W/Q | Rescued nonsense |
| NPIPB6 | 16-28354498-CA-TG | gTG/gCA | V/A | Unchanged |
| APOBR | 16-28508446-CA-TG | gCA/gTG | A/V | Unchanged |
| CTB-134H23.2 | 16-29057391-CA-TG | CAg/TGg | Q/W | Rescued nonsense |
| QPRT | 16-29708350-AC-GT | ACt/GTt | T/V | Changed missense |
| TAOK2 | 16-29998199-CA-TG | aCA/aTG | T/M | Changed missense |
| RNF40 | 16-30779715-CA-TG | CAg/TGg | Q/W | Rescued nonsense |
| KAT8 | 16-31141825-CA-TG | cCA/cTG | P/L | Unchanged |
| ZNF423 | 16-49672520-AC-GT | cGT/cAC | R/H | Unchanged |
| RP11-324D17.1 | 16-54279692-CA-TG | TGg/CAg | W/Q | Rescued nonsense |
| CES1 | 16-55844575-CA-TG | TGc/CAc | C/H | Changed missense |
| CES5A | 16-55897290-CA-GT | cTG/cAC | L/H | Changed missense |
| NLRC5 | 16-57068106-AC-GT | cAC/cGT | H/R | Unchanged |
| CNGB1 | 16-57957215-CA-TG | gTG/gCA | V/A | Unchanged |
| TEPP | 16-58019140-AC-GT | tAC/tGT | Y/C | Unchanged |
| NDRG4 | 16-58545425-CA-TG | tCA/tTG | S/L | Unchanged |
| DUS2 | 16-68088484-CA-TG | tCA/tTG | S/L | Unchanged |
| WWP2 | 16-69970328-CA-TG | aCA/aTG | T/M | Changed missense |
| SF3B3 | 16-70602219-ATC-GTT | AtC/GtT | I/V | Unchanged |
| HP | 16-72094490-CAA-GAT | CaA/GaT | Q/D | Changed missense |
| ZFHX3 | 16-72991714-CA-TG | gTG/gCA | V/A | Unchanged |
| BCAR1 | 16-75269267-AGC-GGT | GcT/AcC | A/T | Unchanged |
| CNTNAP4 | 16-76532582-CA-TG | CAg/TGg | Q/W | Rescued nonsense |
| PKD1L2 | 16-81213377-CA-TG | cTG/cCA | L/P | Unchanged |
| CDH13 | 16-83817018-CA-TG | gCA/gTG | A/V | Unchanged |
| OSGIN1 | 16-83999032-CA-TG | tCA/tTG | S/L | Unchanged |
| KCNG4 | 16-84270540-CA-TG | cTG/cCA | L/P | Unchanged |
| TLDC1 | 16-84520395-CA-TG | TGc/CAc | C/H | Changed missense |
| ZNF469 | 16-88502504-CA-TG | CAt/TGt | H/C | Changed missense |
| ZNF469 | 16-88505634-CA-TG | cCA/cTG | P/L | Unchanged |
| CYBA | 16-88709827-CA-TG | gTG/gCA | V/A | Unchanged |
| PIEZO1 | 16-88804734-AC-GT | GTc/ACc | V/T | Changed missense |
| RP11-36817.4 | 16-89751860-CA-TG | TGc/CAc | C/H | Changed missense |
| CDK10 | 16-89753128-CCA-GCT | CcA/GcT | P/A | Unchanged |
| MC1R | 16-89986607-CA-TG | aCA/aTG | T/M | Changed missense |
| TUBB3 | 16-89986607-CA-TG | aCA/aTG | T/M | Changed missense |
| DEF8 | 16-90020745-CA-GT | CAg/GTg | Q/V | Changed missense |
| GAS8 | 16-90109710-CA-TG | aCA/aTG | T/M | Changed missense |
| MYO1C | 17-1384066-AC-GT | gGT/gAC | G/D | Unchanged |
| MYO1C | 17-1384066-ACC-GCT | GgT/AgC | G/S | Unchanged |
| WDR81 | 17-1637073-CA-TG | cCA/cTG | P/L | Unchanged |
| OR1E2 | 17-3337056-CA-TG | TGc/CAc | C/H | Changed missense |
| ZZEF1 | 17-3981289-CA-TG | gTG/gCA | V/A | Unchanged |
| KIF1C | 17-4926881-CA-TG | cCA/cTG | P/L | Unchanged |
| WSCD1 | 17-6014175-CA-TG | aCA/aTG | T/M | Changed missense |
| ALOX12 | 17-6902759-CA-TG | CAg/TGg | Q/W | Rescued nonsense |
| SLC16A13 | 17-6941624-AC-GT | tAC/tGT | Y/C | Unchanged |
| ACAP1 | 17-7245370-CA-TG | cCA/cTG | P/L | Unchanged |
| AURKB | 17-8108330-CA-TG | aTG/aCA | M/T | Partially changed missense |
| MYH8 | 17-10297657-CA-TG | TGg/CAg | W/Q | Rescued nonsense |
| DNAH9 | 17-11523081-CA-TG | CAa/TGa | Q/* | Unchanged |
| DNAH9 | 17-11648331-AC-GT | aAC/aGT | N/S | Unchanged |
| COX10 | 17-14095308-CA-TG | cCA/cTG | P/L | Unchanged |
| TBC1D26 | 17-15640814-CA-TG | CAc/TGc | H/C | Changed missense |
| TBC1D26 | 17-15640815-AC-GT | cAC/cGT | H/R | Unchanged |
| CCDC144A | 17-16593796-AGC-GGT | AgC/GgT | S/G | Unchanged |
| RP11-219A15.1 | 17-16593796-AGC-GGT | AgC/GgT | S/G | Unchanged |
| LLGL1 | 17-18137141-AGC-GGT | AgC/GgT | S/G | Unchanged |
| LGALS9 | 17-25975991-AC-TG | ACc/TGc | T/C | Changed missense |
| SEZ6 | 17-27284442-CA-TG | aTG/aCA | M/T | Partially changed missense |
| ANKRD13B | 17-27938606-CA-GT | aCA/aGT | T/S | Changed missense |
| NF1 | 17-29556425-CA-TG | cCA/cTG | P/L | Unchanged |
| ARHGAP23 | 17-36646385-CA-TG | gCA/gTG | A/V | Unchanged |
| KRT28 | 17-38956006-CA-TG | TGt/CAt | C/H | Changed missense |
| KRT40 | 17-39137103-CA-TG | aTG/aCA | M/T | Partially changed missense |
| KRTAP4-16P | 17-39258040-AC-GT | GTc/ACc | V/T | Changed missense |
| KRTAP4-12 | 17-39279920-CA-TG | TGc/CAc | C/H | Changed missense |
| KRTAP9-4 | 17-39406244-AC-GT | gAC/gGT | D/G | Unchanged |
| KRT36 | 17-39646021-AC-GT | cGT/cAC | R/H | Unchanged |
| COASY | 17-40716519-CA-TG | aCA/aTG | T/M | Changed missense |
| NBR1 | 17-41361959-CA-TG | CAt/TGt | H/C | Changed missense |
| ASB16 | 17-42248344-CAA-GAT | CaA/GaT | Q/D | Changed missense |
| SPPL2C | 17-43923897-OA-TG | tCA/tTG | S/L | Unchanged |
| KANSL1 | 17-44248736-CA-TG | tTG/tCA | L/S | Unchanged |
| LRRC37A | 17-44374709-CA-TG | cCA/cTG | P/L | Unchanged |
| LRRC37A | 17-44408406-AC-GT | ACg/GTg | T/V | Changed missense |
| EPX | 17-56272388-CGA-TGG | CgA/TgG | R/W | Rescued nonsense |
| TRIM37 | 17-57094674-CA-GT | TGt/ACt | C/T | Changed missense |
| SMG8 | 17-57287453-CA-TG | gCA/gTG | A/V | Unchanged |
| TUBD1 | 17-57963536-CA-TG | aTG/aCA | M/T | Partially changed missense |
| MED13 | 17-60059900-CA-GT | TGt/ACt | C/T | Changed missense |
| TLK2 | 17-60683502-CA-TG | cCA/cTG | P/L | Unchanged |
| ACE | 17-61564051-CA-TG | gCA/gTG | A/V | Unchanged |
| APOH | 17-64216814-AC-GT | cGT/cAC | R/H | Unchanged |
| NOL11 | 17-65739626-CA-TG | gCA/gTG | A/V | Unchanged |
| SLC16A6 | 17-66267530-CA-TG | cTG/cCA | L/P | Unchanged |
| ARSG | 17-66391231-CA-TG | cCA/cTG | P/L | Unchanged |
| FAM20A | 17-66533654-CA-TG | tTG/tCA | L/S | Unchanged |
| ABCA8 | 17-66899568-AC-GT | GTc/ACc | V/T | Changed missense |
| ABCA10 | 17-67178315-CA-TG | aTG/aCA | M/T | Partially changed missense |
| COG1 | 17-71192872-CA-TG | gCA/gTG | A/V | Unchanged |
| TTYH2 | 17-72248482-CA-TG | gCA/gTG | A/V | Unchanged |
| CD300A | 17-72469957-CA-TG | cCA/cTG | P/L | Unchanged |
| CD300LF | 17-72700943-AC-GT | GTc/ACc | V/T | Changed missense |
| NAT9 | 17-72769738-CA-TG | TGc/CAc | C/H | Changed missense |
| GGA3 | 17-73236124-ATC-GTT | GaT/AaC | D/N | Unchanged |
| KIAA0195 | 17-73488792-ATC-GTT | AtC/GtT | I/V | Unchanged |
| UNK | 17-73812908-CA-TG | cCA/cTG | P/L | Unchanged |
| TEN1 | 17-73987651-AC-GT | cAC/cGT | H/R | Unchanged |
| EVPL | 17-74017822-AC-GT | GTg/ACg | V/T | Changed missense |
| QRICH2 | 17-74289704-CA-TG | tTG/tCA | L/S | Unchanged |
| ST6GALNAC1 | 17-74625685-CA-TG | gTG/gCA | V/A | Unchanged |
| FLJ45079 | 17-75878090-AC-GT | GTg/ACg | V/T | Changed missense |
| ENCASE | 17-77078107-CGA-TGG | CgA/TgG | R/W | Rescued nonsense |
| CCDC40 | 17-78073561-CA-TG | cCA/cTG | P/L | Unchanged |
| RNF213 | 17-78261804-CA-TG | cCA/cTG | P/L | Unchanged |
| RPTOR | 17-78865629-CA-TG | gCA/gTG | A/V | Unchanged |
| AZI1 | 17-79171966-CA-TG | gTG/gCA | V/A | Unchanged |
| AZI1 | 17-79171967-AC-GT | GTg/ACg | V/T | Changed missense |
| RP11-1055B8.7 | 17-79409309-AC-GT | ACg/GTg | T/V | Changed missense |
| RP11-1055B8.7 | 17-79409432-CCA-TCG | CcA/TcG | P/S | Unchanged |
| RP11-1055B8.7 | 17-79414307-ACC-GCT | AcC/GcT | T/A | Unchanged |
| RP11-1055B8.7 | 17-79424516-ACC-GCT | AcC/GcT | T/A | Unchanged |
| CCDC57 | 17-80159713-CA-TG | cTG/cCA | L/P | Unchanged |
| OGFOD3 | 17-80352346-AC-TG | gGT/gCA | G/A | Unchanged |
| OGFOD3 | 17-80352346-ACC-TCG | GgT/CgA | G/R | Unchanged |
| HEXDC | 17-80391684-ATC-GTT | AtC/GtT | I/V | Unchanged |
| METRNL | 17-81043037-CCA-TCG | CcA/TcG | P/S | Unchanged |
| METRNL | 17-81043038-CA-TG | cCA/cTG | P/L | Unchanged |
| SMCHD1 | 18-2740713-CA-TG | aCA/aTG | T/M | Changed missense |
| SMCHD1 | 18-2750476-CA-TG | gCA/gTG | A/V | Unchanged |
| TGIF1 | 18-3457538-CA-TG | cCA/cTG | P/L | Unchanged |
| SOGA2 | 18-8784611-CA-TG | CAg/TGg | Q/W | Rescued nonsense |
| TXNDC2 | 18-9887551-ATC-GTT | AtC/GtT | I/V | Unchanged |
| POTEC | 18-14537948-CA-TG | TGt/CAt | C/H | Changed missense |
| NPC1 | 18-21124945-CA-GT | aTG/aAC | M/N | Changed missense |
| HRH4 | 18-22056969-CA-TG | CAt/TGt | H/C | Changed missense |
| CDH2 | 18-25565082-AC-GT | cGT/cAC | R/H | Unchanged |
| DSG2 | 18-29125853-CA-TG | aCA/aTG | T/M | Changed missense |
| TRAPPC8 | 18-29497572-CA-TG | tTG/tCA | L/S | Unchanged |
| MOCOS | 18-33780019-CA-TG | CAc/TGc | H/C | Changed missense |
| MOCOS | 18-33785184-CA-TG | cCA/cTG | P/L | Unchanged |
| SETBP1 | 18-42533129-CA-TG | tCA/tTG | S/L | Unchanged |
| SLC14A1 | 18-43316537-CA-TG | cCA/cTG | P/L | Unchanged |
| CCDC11 | 18-47787601-AC-GT | cGT/cAC | R/H | Unchanged |
| ALPK2 | 18-56247402-AGC-GGT | GcT/AcC | A/T | Unchanged |
| SERPINB4 | 18-61305035-CA-TG | TGt/CAt | C/H | Changed missense |
| SERPINB3 | 18-61325815-AC-GT | GTt/ACt | V/T | Changed missense |
| SERPINB11 | 18-61387276-AC-GT | ACa/GTa | T/V | Changed missense |
| SERPINB8 | 18-61654462-CA-TG | CAc/TGc | H/C | Changed missense |
| DOK6 | 18-67365667-CA-TG | aCA/aTG | T/M | Changed missense |
| SALL3 | 18-76754465-AC-GT | tAC/tGT | Y/C | Unchanged |
| ADNP2 | 18-77893682-CA-TG | gCA/gTG | A/V | Unchanged |
| ODF3L2 | 19-464309-CA-TG | gTG/gCA | V/A | Unchanged |
| GZMM | 19-548573-CA-GT | CAc/GTc | H/V | Changed missense |
| POLRMT | 19-621712-AGC-GGT | GcT/AcC | A/T | Unchanged |
| R3HDM4 | 19-901506-CA-TG | cTG/cCA | L/P | Unchanged |
| ARID3A | 19-929752-CA-TG | cCA/cTG | P/L | Unchanged |
| TMEM259 | 19-1014377-AC-GT | cGT/cAC | R/H | Unchanged |
| ABCA7 | 19-1054058-CTA-TTG | CtA/TtG | L | Unchanged |
| HMHA1 | 19-1085965-CA-TG | aCA/aTG | T/M | Changed missense |
| BTBD2 | 19-1997363-AGC-GGT | GcT/AcC | A/T | Unchanged |
| ZNF554 | 19-2834372-AC-GT | tAC/tGT | Y/C | Unchanged |
| APBA3 | 19-3752873-CA-TG | TGc/CAc | C/H | Changed missense |
| PIAS4 | 19-4029011-AC-GT | cAC/cGT | H/R | Unchanged |
| SIRT6 | 19-4180835-AC-GT | aGT/aAC | S/N | Unchanged |
| MPND | 19-4357554-CA-TG | tCA/tTG | S/L | Unchanged |
| PLIN4 | 19-4511434-AGC-GGT | GcT/AcC | A/T | Unchanged |
| PLIN4 | 19-4511677-AGC-GGT | GcT/AcC | A/T | Unchanged |
| PLIN4 | 19-4512154-CA-TG | gTG/gCA | V/A | Unchanged |
| PLIN4 | 19-4512155-AC-GT | GTg/ACg | V/T | Changed missense |
| PLIN4 | 19-4513558-CA-TG | gTG/gCA | V/A | Unchanged |
| PLIN5 | 19-4525045-CA-TG | TGc/CAc | C/H | Changed missense |
| PTPRS | 19-5214696-CA-TG | TGt/CAt | C/H | Changed missense |
| EMR1 | 19-6913706-AC-GT | ACg/GTg | T/V | Changed missense |
| ZNF557 | 19-7083628-CA-TG | tCA/tTG | S/L | Unchanged |
| CTD-2207O23.3 | 19-7528733-CA-TG | CAg/TGg | Q/W | Rescued nonsense |
| ARHGEF18 | 19-7528733-CA-TG | CAg/TGg | Q/W | Rescued nonsense |
| CTD-3193O13.9 | 19-7934992-CA-TG | tTG/tCA | L/S | Unchanged |
| CTD-3193O13.9 | 19-7935583-CA-TG | tTG/tCA | L/S | Unchanged |
| CTD-3193O13.9 | 19-7936006-CA-TG | aTG/aCA | M/T | Partially changed missense |
| ACTL9 | 19-8808899-CA-TG | gTG/gCA | V/A | Unchanged |
| ACTL9 | 19-8808900-AC-GT | GTg/ACg | V/T | Changed missense |
| MUC16 | 19-9015670-CA-TG | TGc/CAc | C/H | Changed missense |
| MUC16 | 19-9017004-CA-TG | TGg/CAg | W/Q | Rescued nonsense |
| OR1M1 | 19-9204306-CA-TG | cCA/cTG | P/L | Unchanged |
| OR7G3 | 19-9236886-AAC-GAT | GfT/AtC | V/I | Unchanged |
| ZNF561 | 19-9721521-CA-TG | gTG/gCA | V/A | Unchanged |
| RAVER1 | 19-10439711-AC-GT | cGT/cAC | R/H | Unchanged |
| KRI1 | 19-10668672-CA-TG | cTG/cCA | L/P | Unchanged |
| AP1M2 | 19-10694676-AC-TG | GTa/CAa | V/Q | Changed missense |
| SLC44A2 | 19-10742169-CA-TG | CAa/TGa | Q/* | Unchanged |
| RGL3 | 19-11529440-AC-GT | gGT/gAC | G/D | Unchanged |
| ZNF440 | 19-11942362-AC-GT | aAC/aGT | N/S | Unchanged |
| ZNF442 | 19-12461211-CA-TG | aTG/aCA | M/T | Partially changed missense |
| ZNF799 | 19-12502456-CA-TG | cTG/cCA | L/P | Unchanged |
| MAST1 | 19-12985575-CA-TG | tCA/tTG | S/L | Unchanged |
| CD97 | 19-14499537-AAC-GAT | AaC/GaT | N/D | Unchanged |
| ILVBL | 19-15229993-ATC-GTT | GaT/AaC | D/N | Unchanged |
| CYP4F11 | 19-16034713-CA-TG | TGc/CAc | C/H | Changed missense |
| CPAMD8 | 19-17111296-CA-TG | aTG/aCA | M/T | Partially changed missense |
| MYO9B | 19-17283655-ATC-GTT | AtC/GtT | I/V | Unchanged |
| MYO9B | 19-17316955-CA-TG | CAg/TGg | Q/W | Rescued nonsense |
| ANKLE1 | 19-17395Q02-CA-TG | CAg/TGg | Q/W | Rescued nonsense |
| ANO8 | 19-17438642-AAC-GAT | GfT/AtC | V/I | Unchanged |
| TMEM221 | 19-17547291-CA-TG | aTG/aCA | M/T | Partially changed missense |
| PGLS | 19-17628586-CA-TG | aCA/aTG | T/M | Changed missense |
| MAP1S | 19-17837980-CA-TG | gCA/gTG | A/V | Unchanged |
| FCHO1 | 19-17885269-CA-TG | cCA/cTG | P/L | Unchanged |
| IFI30 | 19-18288067-CCA-TCG | CcA/TcG | P/S | Unchanged |
| KIAA1683 | 19-18378218-CA-TG | cTG/cCA | L/P | Unchanged |
| SSBP4 | 19-18542200-AC-GT | aAC/aGT | N/S | Unchanged |
| ZNF737 | 19-20735343-CA-TG | TGt/CAt | C/H | Changed missense |
| ZNF85 | 19-21117760-AC-GT | ACt/GTt | T/V | Changed missense |
| ZNF99 | 19-22952110-CA-TG | TGg/CAg | W/Q | Rescued nonsense |
| RPSAP58 | 19-24010780-AC-GT | ACg/GTg | T/V | Changed missense |
| GPATCH1 | 19-33605299-CA-TG | CAt/TGt | H/C | Changed missense |
| FXYD5 | 19-35649280-CA-TG | cCA/cTG | P/L | Unchanged |
| FAM187B | 19-35719105-CA-TG | TGt/CAt | C/H | Changed missense |
| DMKN | 19-36004106-AC-GT | GTa/ACa | V/T | Changed missense |
| ATP4A | 19-36050968-CA-TG | gTG/gCA | V/A | Unchanged |
| ATP4A | 19-36050969-AC-GT | GTg/ACg | V/T | Changed missense |
| KMT2B | 19-36221352-AC-GT | aAC/aGT | N/S | Unchanged |
| ZNF567 | 19-37210804-AC-GT | tAC/tGT | Y/C | Unchanged |
| ZNF850 | 19-37240509-CA-TG | TGt/CAt | C/H | Changed missense |
| ZNF568 | 19-37441979-CA-TG | CAa/TGa | Q/* | Unchanged |
| ZNF527 | 19-37879588-CA-TG | CAt/TGt | H/C | Changed missense |
| RASGRP4 | 19-38905683-AC-GT | gGT/gAC | G/D | Unchanged |
| RYR1 | 19-38993546-AC-GT | cAC/cGT | H/R | Unchanged |
| CAPN12 | 19-39229088-CA-TG | TGc/CAc | C/H | Changed missense |
| IFNL4 | 19-39739170-CA-TG | TGc/CAc | C/H | Changed missense |
| IFNL2 | 19-39760191-ACC-GCT | AcC/GcT | T/A | Unchanged |
| SHKBP1 | 19-41096188-CA-TG | aCA/aTG | T/M | Changed missense |
| CYP2A6 | 19-41351304-CA-TG | aTG/aCA | M/T | Partially changed missense |
| CYP2A7 | 19-41383200-CA-TG | aTG/aCA | M/T | Partially changed missense |
| CYP2A13 | 19-41600231-CA-TG | aCA/aTG | T/M | Changed missense |
| CYP2F1 | 19-41633926-CA-TG | cCA/cTG | P/L | Unchanged |
| AXL | 19-41743861-AAC-GAT | AaC/GaT | N/D | Unchanged |
| HNRNPUL1 | 19-41809994-CA-TG | cCA/cTG | P/L | Unchanged |
| PSG3 | 19-43243216-CA-TG | tTG/tCA | L/S | Unchanged |
| PSG3 | 19-43243216-CAA-TAG | TtG/CtA | L | Unchanged |
| PSG6 | 19-43414959-AC-GT | GTc/ACc | V/T | Changed missense |
| PSG6 | 19-43420614-CA-TG | cTG/cCA | L/P | Unchanged |
| PSG11 | 19-43529025-AC-GT | GTa/ACa | V/T | Changed missense |
| PSG2 | 19-43585373-CA-TG | cTG/cCA | L/P | Unchanged |
| PSG5 | 19-43679410-AGC-GGT | GcT/AcC | A/T | Unchanged |
| PSG5 | 19-43680299-CA-TG | cTG/cCA | L/P | Unchanged |
| TEX101 | 19-43922060-CA-TG | cCA/cTG | P/L | Unchanged |
| ZNF224 | 19-44610842-AC-GT | ACg/GTg | T/V | Changed missense |
| ZNF285 | 19-44891838-CA-TG | TGt/CAt | C/H | Changed missense |
| ZNF285 | 19-44891849-AC-GT | aGT/aAC | S/N | Unchanged |
| PVRL2 | 19-45389223-CA-TG | cCA/cTG | P/L | Unchanged |
| HIF3A | 19-46811983-CA-TG | aCA/aTG | T/M | Changed missense |
| HIF3A | 19-46823701-CA-TG | CAg/TGg | Q/W | Rescued nonsense |
| PRKD2 | 19-47177912-CA-TG | gTG/gCA | V/A | Unchanged |
| PRKD2 | 19-47177913-AC-GT | GTg/ACg | V/T | Changed missense |
| GLTSCR2 | 19-48255803-CA-TG | gCA/gTG | A/V | Unchanged |
| GRIN2D | 19-48946404-CA-TG | cCA/cTG | P/L | Unchanged |
| FUT2 | 19-49206416-CA-TG | gCA/gTG | A/V | Unchanged |
| FUT2 | 19-49207205-CA-TG | aCA/aTG | T/M | Changed missense |
| KCNA7 | 19-49575618-ACC-GCT | GgT/AgC | G/S | Unchanged |
| HRC | 19-49658099-AC-GT | cGT/cAC | R/H | Unchanged |
| NUP62 | 19-50411742-ATC-GTT | GaT/AaC | D/N | Unchanged |
| MYH14 | 19-50762417-CA-TG | cCA/cTG | P/L | Unchanged |
| KLK1 | 19-51323501-ATC-GTT | GaT/AaC | D/N | Unchanged |
| KLK3 | 19-51359502-CA-TG | gCA/gTG | A/V | Unchanged |
| KLK10 | 19-51519235-CA-TG | cTG/cCA | L/P | Unchanged |
| SIGLEC12 | 19-52000155-AGC-GGT | GcT/AcC | A/T | Unchanged |
| SIGLEC5 | 19-52133292-AC-GT | GTt/ACt | V/T | Changed missense |
| ZNF480 | 19-52825234-CA-TG | tCA/tTG | S/L | Unchanged |
| ZNF534 | 19-52942445-CA-TG | CAc/TGc | H/C | Changed missense |
| ZNF808 | 19-53056374-CA-TG | CAc/TGc | H/C | Changed missense |
| ZNF701 | 19-53085565-AC-TG | ACt/TGt | T/C | Changed missense |
| ZNF600 | 19-53270383-CA-TG | TGt/CAt | C/H | Changed missense |
| ZNF28 | 19-53303526-CA-TG | aTG/aCA | M/T | Partially changed missense |
| ERVV-2 | 19-53554074-CA-TG | CAa/TGa | Q/* | Unchanged |
| VN1R2 | 19-53761764-ACC-GCT | AcC/GcT | T/A | Unchanged |
| BIRC8 | 19-53793574-AAC-GAT | GtT/AtC | V/I | Unchanged |
| ZNF765 | 19-53911973-AGC-TGG | AgC/TgG | S/W | Changed missense |
| LILRB3 | 19-54721215-CA-TG | gTG/gCA | V/A | Unchanged |
| LILRB3 | 19-54725744-CA-TG | TGg/CAg | W/Q | Rescued nonsense |
| LILRA6 | 19-54745906-CA-TG | aTG/aCA | M/T | Partially changed missense |
| LILRB2 | 19-54782385-CA-TG | gTG/gCA | V/A | Unchanged |
| LILRB2 | 19-54782406-AC-GT | cGT/cAC | R/H | Unchanged |
| LILRA4 | 19-54844856-CA-TG | TGg/CAg | W/Q | Rescued nonsense |
| LILRA4 | 19-54848383-CA-TG | gTG/gCA | V/A | Unchanged |
| LILRA1 | 19-55105726-CA-TG | aCA/aTG | T/M | Changed missense |
| LILRB1 | 19-55144184-AGC-GGT | AgC/GgT | S/G | Unchanged |
| LILRB4 | 19-55179216-CA-TG | gCA/gTG | A/V | Unchanged |
| KIR2DL1 | 19-55295125-AC-GT | ACa/GTa | T/V | Changed missense |
| PTPRH | 19-55708555-CAA-TAG | TtG/CtA | L | Unchanged |
| PTPRH | 19-55713614-ATC-GTT | GaT/AaC | D/N | Unchanged |
| ZNF628 | 19-55993260-ACC-GCT | AcC/GcT | T/A | Unchanged |
| ZNF628 | 19-55994652-CA-TG | CAg/TGg | Q/W | Rescued nonsense |
| SBK2 | 19-56047447-CA-TG | TGc/CAc | C/H | Changed missense |
| CCDC106 | 19-56162774-AGC-GGT | AgC/GgT | S/G | Unchanged |
| NLRP13 | 19-56407380-ATC-TTG | GaT/CaA | D/Q | Changed missense |
| ZSCAN5C | 19-56717341-CA-TG | tCA/tTG | S/L | Unchanged |
| ZSCAN5C | 19-56717352-CA-TG | CAg/TGg | Q/W | Rescued nonsense |
| ZSCAN5C | 19-56718584-CA-TG | CAa/TGa | Q/* | Unchanged |
| ZNF71 | 19-57133632-AC-GT | tAC/tGT | Y/C | Unchanged |
| ZNF460 | 19-57802805-AC-GT | aAC/aGT | N/S | Unchanged |
| ZNF749 | 19-57955243-CA-TG | CAg/TGg | Q/W | Rescued nonsense |
| ZSCAN4 | 19-58189713-CTA-TTG | CtA/TtG | L | Unchanged |
| ZNF544 | 19-58774070-CA-TG | CAa/TGa | Q/* | Unchanged |
| SNTG2 | 2-1271230-ATC-GTT | AtC/GtT | I/V | Unchanged |
| CMPK2 | 2-7005369-AC-GT | tGT/tAC | C/Y | Unchanged |
| TAF1B | 2-10045013-AC-GT | tAC/tGT | Y/C | Unchanged |
| DDX1 | 2-15747392-AC-GT | aAC/aGT | N/S | Unchanged |
| MSGN1 | 2-17998095-CA-TG | CAc/TGc | H/C | Changed missense |
| GPR113 | 2-26536692-AGC-GGT | GcT/AcC | A/T | Unchanged |
| EMILIN1 | 2-27306045-CA-TG | CAa/TGa | Q/* | Unchanged |
| CAD | 2-27460967-CA-TG | gCA/gTG | A/V | Unchanged |
| DNAJC5G | 2-27500852-AC-TG | tAC/tTG | Y/L | Rescued nonsense |
| WDR43 | 2-29152526-CCA-GCT | CcA/GcT | P/A | Unchanged |
| FAM179A | 2-29226511-CA-TG | CAg/TGg | Q/W | Rescued nonsense |
| FAM179A | 2-29240059-CA-TG | CAg/TGg | Q/W | Rescued nonsense |
| CLIP4 | 2-29356668-CA-TG | aCA/aTG | T/M | Changed missense |
| TTC27 | 2-32855613-CTA-TTG | CtA/TtG | L | Unchanged |
| LTBP1 | 2-33447201-AC-GT | aAC/aGT | N/S | Unchanged |
| RASGRP3 | 2-33764176-AC-GT | ACg/GTg | T/V | Changed missense |
| CYP1B1 | 2-38298150-ATC-GTT | GaT/AaC | D/N | Unchanged |
| CYP1B1 | 2-38298202-AC-TG | GTg/CAg | V/Q | Changed missense |
| ATL2 | 2-38604349-CCA-TCG | TgG/CgA | W/R | Rescued nonsense |
| SOCS5 | 2-46986415-CA-TG | aCA/aTG | T/M | Changed missense |
| MSH6 | 2-48018080-CA-TG | cCA/cTG | P/L | Unchanged |
| ERLEC1 | 2-54035445-CAA-TAG | CaA/TaG | Q/* | Unchanged |
| EML6 | 2-55040425-CA-TG | cCA/cTG | P/L | Unchanged |
| USP34 | 2-61575307-CA-TG | aTG/aCA | M/T | Partially changed missense |
| FAM161A | 2-62066927-AC-GT | tGT/tAC | C/Y | Unchanged |
| CEP68 | 2-65298838-CA-TG | aCA/aTG | T/M | Changed missense |
| CD207 | 2-71058834-CA-TG | gTG/gCA | V/A | Unchanged |
| CYP26B1 | 2-72361959-CA-TG | tTG/tCA | L/S | Unchanged |
| NAT8 | 2-73868150-AC-GT | tGT/tAC | C/Y | Unchanged |
| NAT8 | 2-73868151-CA-TG | TGt/CAt | C/H | Changed missense |
| LOXL3 | 2-74761469-CA-GT | TGt/ACt | C/T | Changed missense |
| HK2 | 2-75099475-CAA-GAT | CaA/GaT | Q/D | Changed missense |
| HK2 | 2-75113656-AC-GT | aAC/aGT | N/S | Unchanged |
| VAMP8 | 2-85808735-CGA-TGG | CgA/TgG | R/W | Rescued nonsense |
| IGKV4-1 | 2-89185467-AC-GT | aAC/aGT | N/S | Unchanged |
| IGKV5-2 | 2-89197074-AC-GT | aAC/aGT | N/S | Unchanged |
| IGKV5-2 | 2-89197190-CCA-TCG | CcA/TcG | P/S | Unchanged |
| IGKV1-5 | 2-89246936-AC-GT | aGT/aAC | S/N | Unchanged |
| IGKV1-9 | 2-89309660-CA-TG | TGg/CAg | W/Q | Rescued nonsense |
| IGKV3-11 | 2-89326866-AAC-GAT | GtT/AtC | V/I | Unchanged |
| IGKV1-12 | 2-89339910-CCA-TCG | TgG/CgA | W/R | Rescued nonsense |
| IGKV3-15 | 2-89384829-AGC-GGT | GcT/AcC | A/T | Unchanged |
| IGKV2-24 | 2-89475830-CA-TG | aTG/aCA | M/T | Partially changed missense |
| IGKV2D-28 | 2-89998840-CA-TG | CAg/TGg | Q/W | Rescued nonsense |
| FAHD2A | 2-96072753-CGA-TGG | CgA/TgG | R/W | Rescued nonsense |
| ITPRIPL1 | 2-96993883-CA-TG | cCA/cTG | P/L | Unchanged |
| FAM178B | 2-97638037-CA-TG | gTG/gCA | V/A | Unchanged |
| FAM178B | 2-97638038-AC-GT | GTg/ACg | V/T | Changed missense |
| FAHD2B | 2-97755996-CCA-TCG | TgG/CgA | W/R | Rescued nonsense |
| ANKRD36 | 2-979Q9726-CA-TG | gCA/gTG | A/V | Unchanged |
| RFX8 | 2-102018907-CA-TG | aTG/aCA | M/T | Partially changed missense |
| TGFBRAP1 | 2-105924510-AC-GT | cGT/cAC | R/H | Unchanged |
| RANBP2 | 2-109374951-CA-TG | tCA/tTG | S/L | Unchanged |
| MERTK | 2-112705142-CA-TG | cCA/cTG | P/L | Unchanged |
| MERTK | 2-112765972-CA-TG | tCA/tTG | S/L | Unchanged |
| GLI2 | 2-121747427-CCA-TCG | CcA/TcG | P/S | Unchanged |
| TUBA3E | 2-130949552-CA-TG | TGg/CAg | W/Q | Rescued nonsense |
| POTEI | 2-131220830-CA-TG | aTG/aCA | M/T | Partially changed missense |
| TUBA3D | 2-132237982-CA-TG | aCA/aTG | T/M | Changed missense |
| TUBA3D | 2-132238284-ACC-GCT | AcC/GcT | T/A | Unchanged |
| NCKAP5 | 2-133541574-AC-GT | GTc/ACc | V/T | Changed missense |
| THSD7B | 2-138417312-CA-TG | aCA/aTG | T/M | Changed missense |
| NMI | 2-152132128-CAA-TAG | TtG/CtA | L | Unchanged |
| RIF1 | 2-152322240-AC-TG | gAC/gTG | D/V | Partially changed missense |
| RIF1 | 2-152322332-AAC-GAT | AaC/GaT | N/D | Unchanged |
| NEB | 2-152483630-AGC-GGT | GcT/AcC | A/T | Unchanged |
| NEB | 2-152490457-CA-TG | TGc/CAc | C/H | Changed missense |
| TANC1 | 2-160086691-CA-TG | aCA/aTG | T/M | Changed missense |
| COBLL1 | 2-165551659-CA-GT | TGg/ACg | W/T | Changed missense |
| SCN9A | 2-167099157-CA-TG | TGg/CAg | W/Q | Rescued nonsense |
| METTL8 | 2-172180770-CA-TG | TGa/CAa | */Q | Unchanged |
| TTN | 2-179518002-CA-TG | tTG/tCA | L/S | Unchanged |
| TTN | 2-179620950-AC-GT | gGT/gAC | G/D | Unchanged |
| ZNF804A | 2-185800904-CA-GT | CAa/GTa | Q/V | Changed missense |
| ZNF804A | 2-185802362-CA-TG | CAc/TGc | H/C | Changed missense |
| FSIP2 | 2-186678632-AC-GT | ACt/GTt | T/V | Changed missense |
| ITGAV | 2-187540562-CA-GT | CAt/GTt | H/V | Changed missense |
| DIRC1 | 2-189599372-CA-TG | tTG/tCA | L/S | Unchanged |
| SLC40A1 | 2-190430177-AAC-GAT | GtT/AtC | V/I | Unchanged |
| MFSD6 | 2-191302004-AC-GT | ACa/GTa | T/V | Changed missense |
| NOP58 | 2-203149164-CCA-TCG | CcA/TcG | P/S | Unchanged |
| CPO | 2-207833982-CA-TG | aCA/aTG | T/M | Changed missense |
| CRYGB | 2-209010694-CA-GT | TGc/ACc | C/T | Changed missense |
| C2orf80 | 2-209036777-CA-TG | TGc/CAc | C/H | Changed missense |
| CPS1 | 2-211456637-ACC-GCT | AcC/GcT | T/A | Unchanged |
| ERBB4 | 2-212522512-CA-TG | TGg/CAg | W/Q | Rescued nonsense |
| TNS1 | 2-218674696-AC-GT | GTc/ACc | V/T | Changed missense |
| TNS1 | 2-218683153-CA-TG | TGg/CAg | W/Q | Rescued nonsense |
| RUFY4 | 2-218940395-CA-TG | CAa/TGa | Q/* | Unchanged |
| USP37 | 2-219319656-CA-TG | tTG/tCA | L/S | Unchanged |
| DNPEP | 2-220250146-CA-TG | gTG/gCA | V/A | Unchanged |
| SPEG | 2-220355528-CA-TG | CAc/TGc | H/C | Changed missense |
| TMEM198 | 2-220414015-AC-GT | gAC/gGT | D/G | Unchanged |
| SGPP2 | 2-223423360-CCA-TCG | CcA/TcG | P/S | Unchanged |
| SGPP2 | 2-223423361-CA-TG | cCA/cTG | P/L | Unchanged |
| COL4A3 | 2-228131169-AC-GT | cAC/cGT | H/R | Unchanged |
| AGFG1 | 2-228398421-CA-TG | gCA/gTG | A/V | Unchanged |
| AGFG1 | 2-228416711-CA-TG | aCA/aTG | T/M | Changed missense |
| SP110 | 2-231037616-ACC-GCT | GgT/AgC | G/S | Unchanged |
| SP140L | 2-231256811-CA-TG | gCA/gTG | A/V | Unchanged |
| CHRND | 2-233390936-CA-TG | cCA/cTG | P/L | Unchanged |
| USP40 | 2-234399822-CA-TG | cTG/cCA | L/P | Unchanged |
| UGT1A5 | 2-234621668-CA-TG | CAg/TGg | Q/W | Rescued nonsense |
| UGT1A4 | 2-234627743-AC-GT | ACg/GTg | T/V | Changed missense |
| UGT1A3 | 2-234638248-CA-TG | gCA/gTG | A/V | Unchanged |
| UGT1A8 | 2-234638248-CA-TG | gCA/gTG | A/V | Unchanged |
| SH3BP4 | 2-235949919-CA-TG | cCA/cTG | P/L | Unchanged |
| RAB17 | 2-238494741-CA-TG | gTG/gCA | V/A | Unchanged |
| LRRFIP1 | 2-238672326-AC-GT | gAC/gGT | D/G | Unchanged |
| ESPNL | 2-239039840-CA-TG | CAg/TGg | Q/W | Rescued nonsense |
| ASB1 | 2-239344573-AC-GT | aAC/aGT | N/S | Unchanged |
| AC079612.1 | 2-240500412-CA-TG | cCA/cTG | P/L | Unchanged |
| OR6B2 | 2-240969311-CA-TG | TGt/CAt | C/H | Changed missense |
| PRR21 | 2-240981486-CA-TG | TGc/CAc | C/H | Changed missense |
| PRR21 | 2-240981659-AC-GT | cGT/cAC | R/H | Unchanged |
| PRR21 | 2-240982130-CA-TG | aTG/aCA | M/T | Partially changed missense |
| PRR21 | 2-240982200-CA-TG | TGt/CAt | C/H | Changed missense |
| PRR21 | 2-240982256-CA-TG | aTG/aCA | M/T | Partially changed missense |
| GPC1 | 2-241405478-AC-GT | gAC/gGT | D/G | Unchanged |
| CAPN10 | 2-241531477-CCA-TCG | CcA/TcG | P/S | Unchanged |
| C2orf54 | 2-241828033-CA-TG | cTG/cCA | L/P | Unchanged |
| AC104809.3 | 2-241898823-CA-TG | CAg/TGg | Q/W | Rescued nonsense |
| SDCBP2 | 20-1293008-CA-TG | gTG/gCA | V/A | Unchanged |
| SIRPG | 20-1616205-CA-TG | gTG/gCA | V/A | Unchanged |
| SIRPG | 20-1616206-AC-GT | GTg/ACg | V/T | Changed missense |
| SIRPA | 20-1895819-AC-GT | ACt/GTt | T/V | Changed missense |
| SMOX | 20-4164282-CA-TG | aCA/aTG | T/M | Changed missense |
| BMP2 | 20-6751033-CA-TG | tCA/tTG | S/L | Unchanged |
| PLCB1 | 20-8770822-CTA-TTG | CtA/TtG | L | Unchanged |
| PLCB4 | 20-9404443-AAC-GAT | AaC/GaT | N/D | Unchanged |
| SPTLC3 | 20-13053034-CA-TG | tCA/tTG | S/L | Unchanged |
| KIF16B | 20-16359566-CA-TG | aTG/aCA | M/T | Partially changed missense |
| CST5 | 20-23860177-CA-TG | TGt/CAt | C/H | Changed missense |
| CEP250 | 20-34057798-CA-TG | aCA/aTG | T/M | Changed missense |
| R3HDML | 20-42965862-AC-GT | aAC/aGT | N/S | Unchanged |
| PABPC1L | 20-43566786-CA-TG | tCA/tTG | S/L | Unchanged |
| WFDC3 | 20-44417643-AC-GT | gGT/gAC | G/D | Unchanged |
| FAM65C | 20-49214155-CA-TG | cTG/cCA | L/P | Unchanged |
| PCK1 | 20-56137806-CA-TG | tCA/tTG | S/L | Unchanged |
| PCK1 | 20-56137833-CA-TG | tCA/tTG | S/L | Unchanged |
| C20orf85 | 20-56735759-ATC-GTT | AtC/GtT | I/V | Unchanged |
| LAMA5 | 20-60905877-CA-TG | aTG/aCA | M/T | Partially changed missense |
| OGFR | 20-61444628-CA-TG | cCA/cTG | P/L | Unchanged |
| SLC17A9 | 20-61588158-CA-TG | aCA/aTG | T/M | Changed missense |
| SLC17A9 | 20-61594679-AC-GT | aAC/aGT | N/S | Unchanged |
| TNFRSF6B | 20-62328373-CTA-TTG | CtA/TtG | L | Unchanged |
| POTED | 21-14982634-CA-TG | CAc/TGc | H/C | Changed missense |
| SYNJ1 | 21-34003930-AC-GT | GTt/ACt | V/T | Changed missense |
| SON | 21-34925142-CA-TG | tCA/tTG | S/L | Unchanged |
| SLC5A3 | 21-35467645-AC-GT | ACa/GTa | T/V | Changed missense |
| DOPEY2 | 21-37618179-CA-TG | CAg/TGg | Q/W | Rescued nonsense |
| KCNJ6 | 21-38997497-AC-TG | gGT/gCA | G/A | Unchanged |
| ETS2 | 21-40191637-CA-TG | cCA/cTG | P/L | Unchanged |
| MX1 | 21-42824660-CA-TG | gCA/gTG | A/V | Unchanged |
| PRDM15 | 21-43221481-CGA-TGG | TcG/CcA | S/P | Unchanged |
| RRP1B | 21-45107517-CA-TG | cCA/cTG | P/L | Unchanged |
| PWP2 | 21-45540918-CA-TG | aCA/aTG | T/M | Changed missense |
| PWP2 | 21-45550492-CA-TG | CAc/TGc | H/C | Changed missense |
| TRPM2 | 21-45844750-CA-TG | CAa/TGa | Q/* | Unchanged |
| KRTAP10-6 | 21-46012363-CA-TG | aTG/aCA | M/T | Unchanged |
| KRTAP10-10 | 21-46057551-CCA-TCG | CcA/TcG | P/S | Unchanged |
| KRTAP10-11 | 21-46067217-CA-TG | gCA/gTG | A/V | Unchanged |
| C21orf67 | 21-46355710-AC-GT | cGT/cAC | R/H | Unchanged |
| COL18A1 | 21-46927488-CA-TG | aCA/aTG | T/M | Changed missense |
| COL18A1 | 21-46932256-CA-TG | CAt/TGt | H/C | Changed missense |
| PCNT | 21-47831797-AC-GT | cAC/cGT | H/R | Unchanged |
| PCNT | 21-47841940-CA-TG | CAa/TGa | Q/* | Unchanged |
| PCNT | 21-47851752-CA-TG | CAg/TGg | Q/W | Rescued nonsense |
| PCNT | 21-47855875-CA-TG | aCA/aTG | T/M | Changed missense |
| POTEH | 22-16287337-AGC-GGT | GcT/AcC | A/T | Unchanged |
| BCL2L13 | 22-18209612-CA-TG | tCA/tTG | S/L | Unchanged |
| BCL2L13 | 22-18209716-AAC-GAT | AaC/GaT | N/D | Unchanged |
| TUBA8 | 22-18606977-CA-TG | aCA/aTG | T/M | Changed missense |
| AC008132.13 | 22-18834965-CA-TG | gCA/gTG | A/V | Unchanged |
| PRODH | 22-18912677-CA-TG | TGg/CAg | W/Q | Rescued nonsense |
| TSSK2 | 22-19119750-AC-GT | ACg/GTg | T/V | Changed missense |
| TBX1 | 22-19753960-CA-TG | gCA/gTG | A/V | Unchanged |
| ARVCF | 22-19969105-CA-TG | gTG/gCA | V/A | Unchanged |
| P2RX6 | 22-21377275-AC-GT | ACc/GTc | T/V | Changed missense |
| IGLV4-69 | 22-22385708-AC-TG | tAC/tTG | Y/L | Rescued nonsense |
| IGLV4-69 | 22-22385723-AC-GT | aAC/aGT | N/S | Unchanged |
| IGLV4-69 | 22-22385779-AGC-GGT | AgC/GgT | S/G | Unchanged |
| IGLV9-49 | 22-22698013-AAC-GAT | AaC/GaT | N/D | Unchanged |
| IGLV3-25 | 22-23029532-CA-TG | gCA/gTG | A/V | Unchanged |
| IGLV2-14 | 22-23101669-AC-GT | tAC/tGT | Y/C | Unchanged |
| IGLV3-10 | 22-23154570-CCA-TCG | CcA/TcG | P/S | Unchanged |
| IGLV3-1 | 22-23223438-AGC-TGG | AgC/TgG | S/W | Changed missense |
| RGL4 | 22-24034287-CA-TG | CAg/TGg | Q/W | Rescued nonsense |
| PIWIL3 | 22-25145470-CA-TG | TGt/CAt | C/H | Changed missense |
| C22orf31 | 22-29456698-CA-TG | TGt/CAt | C/H | Changed missense |
| THOC5 | 22-29913271-AC-GT | GTt/ACt | V/T | Changed missense |
| CABP7 | 22-30123750-AC-GT | aAC/aGT | N/S | Unchanged |
| SLC35E4 | 22-31032918-CCA-TCG | CcA/TcG | P/S | Unchanged |
| C22orf42 | 22-32554984-CA-TG | cTG/cCA | L/P | Unchanged |
| RFPL3 | 22-32754230-ACC-GCT | AcC/GcT | T/A | Unchanged |
| CSF2RB | 22-37333793-CA-TG | cCA/cTG | P/L | Unchanged |
| TRIOBP | 22-38122461-CA-TG | CAc/TGc | H/C | Changed missense |
| SLC16A8 | 22-38474695-CA-TG | gTG/gCA | V/A | Unchanged |
| APOBEC3D | 22-39425474-AC-GT | ACa/GTa | T/V | Changed missense |
| MCHR1 | 22-41075694-AC-GT | aAC/aGT | N/S | Unchanged |
| CYP2D6 | 22-42523942-CA-TG | TGc/CAc | C/H | Changed missense |
| TSPO | 22-43558926-AC-GT | ACg/GTg | T/V | Changed missense |
| SCUBE1 | 22-43610106-CGA-TGG | TcG/CcA | S/P | Unchanged |
| PNPLA5 | 22-44282275-CA-TG | TGg/CAg | W/Q | Rescued nonsense |
| PRR5-ARHGAP8 | 22-45198008-CA-TG | cCA/cTG | P/L | Unchanged |
| ARHGAP8 | 22-45198008-CA-TG | cCA/cTG | P/L | Unchanged |
| PRR5-ARHGAP8 | 22-45258456-CA-TG | gCA/gTG | A/V | Unchanged |
| ARHGAP8 | 22-45258456-CA-TG | gCA/gTG | A/V | Unchanged |
| FBLN1 | 22-45923826-CA-TG | CAg/TGg | Q/W | Rescued nonsense |
| PKDREJ | 22-46652998-ATC-GTT | GaT/AaC | D/N | Unchanged |
| PKDREJ | 22-46656241-CA-TG | gTG/gCA | V/A | Unchanged |
| CELSR1 | 22-46929691-CA-TG | TGc/CAc | C/H | Changed missense |
| ZBED4 | 22-50277881-CCA-TCG | CcA/TcG | P/S | Unchanged |
| ZBED4 | 22-50277882-CA-TG | cCA/cTG | P/L | Unchanged |
| ZBED4 | 22-50278437-CA-TG | cCA/cTG | P/L | Unchanged |
| MOV10L1 | 22-50582625-CA-TG | CAg/TGg | Q/W | Rescued nonsense |
| TUBGCP6 | 22-50664611-CA-TG | tTG/tCA | L/S | Unchanged |
| TUBGCP6 | 22-50665476-AGC-GGT | GcT/AcC | A/T | Unchanged |
| KLHDC7B | 22-50988192-CA-TG | CAg/TGg | H/C | Changed missense |
| CHL1 | 3-391100-AC-GT | ACa/GTa | T/V | Changed missense |
| CNTN4 | 3-3076442-CA-TG | cCA/cTG | P/L | Unchanged |
| TRNT1 | 3-3189278-CA-TG | gCA/gTG | A/V | Unchanged |
| LRRN1 | 3-3886579-CA-TG | gCA/gTG | A/V | Unchanged |
| IL17RE | 3-9952705-CA-TG | aCA/aTG | T/M | Changed missense |
| IL17RC | 3-9969880-AC-GT | ACg/GTg | T/V | Changed missense |
| IL17RC | 3-9970030-CA-TG | CAa/TGa | Q/* | Unchanged |
| TATDN2 | 3-10302055-CA-TG | CAt/TGt | H/C | Changed missense |
| RP11-438J1.1 | 3-10302055-CA-TG | CAt/TGt | H/C | Changed missense |
| TATDN2 | 3-10312505-CTA-TTG | CtA/TtG | L | Unchanged |
| RP11-438J1.1 | 3-10312505-CTA-TTG | CtA/TtG | L | Unchanged |
| SLC6A11 | 3-10976875-CA-TG | aCA/aTG | T/M | Changed missense |
| CAND2 | 3-12856855-CA-TG | CAg/TGg | Q/W | Rescued nonsense |
| CAND2 | 3-12858394-CA-TG | CAg/TGg | H/C | Changed missense |
| FBLN2 | 3-13670480-CA-TG | aCA/aTG | T/M | Changed missense |
| FBLN2 | 3-13670536-ACC-GCT | AcC/GcT | T/A | Unchanged |
| TMEM43 | 3-14180704-AGC-GGT | AgC/GgT | S/G | Unchanged |
| RP11-434D12.1 | 3-14180704-AGC-GGT | AgC/GgT | S/G | Unchanged |
| FGD5 | 3-14862947-CA-TG | cCA/cTG | P/L | Unchanged |
| SH3BP5 | 3-15311325-AC-GT | cGT/cAC | R/H | Unchanged |
| KCNH8 | 3-19574944-CA-TG | CAg/TGg | Q/W | Rescued nonsense |
| ZNF860 | 3-32031134-CA-TG | tCA/tTG | S/L | Unchanged |
| GLB1 | 3-33055720-CA-TG | TGc/CAc | C/H | Changed missense |
| PDCD6IP | 3-33907944-CA-TG | cCA/cTG | P/L | Unchanged |
| DLEC1 | 3-38163574-CA-TG | cCA/cTG | P/L | Unchanged |
| ACVR2B | 3-38524741-AC-GT | aAC/aGT | N/S | Unchanged |
| SCN5A | 3-38592406-ATC-GTT | GaT/AaC | D/N | Unchanged |
| CX3CR1 | 3-39307254-AAC-GAT | GtT/AtC | V/I | Unchanged |
| ZBTB47 | 3-42700629-CA-TG | cCA/cTG | P/L | Unchanged |
| FAM198A | 3-43074870-CA-TG | gCA/gTG | A/V | Unchanged |
| FAM198A | 3-43095100-CA-TG | CAg/TGg | Q/W | Rescued nonsense |
| ZNF852 | 3-44540715-CA-TG | aTG/aCA | M/T | Partially changed missense |
| SACM1L | 3-45754684-CA-TG | cCA/cTG | P/L | Unchanged |
| PTPN23 | 3-47453607-AC-TG | aAC/aTG | N/M | Changed missense |
| CCDC71 | 3-49200626-CA-TG | TGg/CAg | W/Q | Rescued nonsense |
| PARP3 | 3-51978219-CA-TG | CAc/TGc | H/C | Changed missense |
| ALAS1 | 3-52237968-ATC-GTT | AtC/GtT | I/V | Unchanged |
| DNAH1 | 3-52430697-CA-TG | CAt/TGt | H/C | Changed missense |
| ITIH1 | 3-52821010-CA-TG | CAg/TGg | Q/W | Rescued nonsense |
| ITIH4 | 3-52864635-AC-GT | cGT/cAC | R/H | Unchanged |
| DCP1A | 3-53326340-AC-GT | GTg/ACg | V/T | Changed missense |
| CCDC66 | 3-56597952-ACC-GCT | AcC/GcT | T/A | Unchanged |
| FLNB | 3-58112439-CA-TG | gCA/gTG | A/V | Unchanged |
| PTPRG | 3-62189526-CA-TG | gCA/gTG | A/V | Unchanged |
| SLC25A26 | 3-66419941-CA-TG | gCA/gTG | A/V | Unchanged |
| FRMD4B | 3-69246150-CAA-TAG | TtG/CtA | L | Unchanged |
| EBLN2 | 3-73111972-CA-TG | cCA/cTG | P/L | Unchanged |
| ZNF717 | 3-75787869-CA-TG | TGc/CAc | C/H | Changed missense |
| ZNF717 | 3-75790879-CA-TG | gTG/gCA | V/A | Unchanged |
| OR5H8P | 3-98031112-CA-TG | gCA/gTG | A/V | Unchanged |
| OR5H8P | 3-98031538-CA-TG | gCA/gTG | A/V | Unchanged |
| GPR15 | 3-98250966-CCA-TCG | CcA/TcG | P/S | Unchanged |
| CEP97 | 3-101476757-CA-TG | gCA/gTG | A/V | Unchanged |
| GUCA1C | 3-108639422-AC-GT | GTt/ACt | V/T | Changed missense |
| WDR52 | 3-113046640-ATC-GTT | GaT/AaC | D/N | Unchanged |
| KIAA2018 | 3-113373929-CA-TG | gTG/gCA | V/A | Unchanged |
| SLC15A2 | 3-121646640-CA-TG | gCA/gTG | A/V | Unchanged |
| PARP15 | 3-122354009-AC-GT | cAC/cGT | H/R | Unchanged |
| HEG1 | 3-124728625-CA-TG | aTG/aCA | M/T | Partially changed missense |
| PLXNA1 | 3-126726704-CA-TG | aCA/aTG | T/M | Changed missense |
| PODXL2 | 3-127379268-ACC-GCT | AcC/GcT | T/A | Unchanged |
| PLXND1 | 3-129281979-AC-GT | aGT/aAC | S/N | Unchanged |
| COL6A5 | 3-130162394-CA-TG | CAa/TGa | Q/* | Unchanged |
| MRPL3 | 3-131188573-CA-TG | aTG/aCA | M/T | Partially changed missense |
| AGTR1 | 3-148459883-CA-TG | cCA/cTG | P/L | Unchanged |
| CPA3 | 3-148597611-AC-GT | ACg/GTg | T/V | Changed missense |
| HPS3 | 3-148863150-CA-TG | aCA/aTG | T/M | Changed missense |
| IGSF10 | 3-151166872-AC-GT | aGT/aAC | S/N | Unchanged |
| MME | 3-154864987-AAC-GAT | AaC/GaT | N/D | Unchanged |
| PLCH1 | 3-155314034-AC-GT | aGT/aAC | S/N | Unchanged |
| RARRES1 | 3-158450169-CCA-GCT | TgG/AgC | W/S | Changed missense |
| PLD1 | 3-171395468-AC-GT | cGT/cAC | R/H | Unchanged |
| KLHL24 | 3-183368890-AC-GT | cAC/cGT | H/R | Unchanged |
| HTR3E | 3-183824369-CA-TG | tCA/tTG | S/L | Unchanged |
| EPHB3 | 3-184289151-CA-TG | aCA/aTG | T/M | Changed missense |
| EHHADH | 3-184910041-CA-TG | tTG/tCA | L/S | Unchanged |
| RTP4 | 3-187088655-AC-GT | ACa/GTa | T/V | Changed missense |
| LPP | 3-188590444-CGA-TGG | CgA/TgG | R/W | Rescued nonsense |
| PYDC2 | 3-191179192-CA-TG | CAa/TGa | Q/* | Unchanged |
| ATP13A4 | 3-193183877-CA-TG | cTG/cCA | L/P | Unchanged |
| CPN2 | 3-194062517-AGC-GGT | GcT/AcC | A/T | Unchanged |
| MUC4 | 3-195505944-CA-TG | gTG/gCA | V/A | Unchanged |
| MUC4 | 3-195506674-AC-GT | GTt/ACt | V/T | Changed missense |
| MUC4 | 3-195507144-CA-TG | gTG/gCA | V/A | Unchanged |
| MUC4 | 3-195507226-AC-GT | GTc/ACc | V/T | Changed missense |
| MUC4 | 3-195507274-AC-GT | GTc/ACc | V/T | Changed missense |
| MUC4 | 3-195509823-ACC-TCG | GgT/CgA | G/R | Unchanged |
| MUC4 | 3-195509832-CA-TG | gTG/gCA | V/A | Unchanged |
| MUC4 | 3-195509976-CA-TG | gTG/gCA | V/A | Unchanged |
| MUC4 | 3-195510024-CA-TG | gTG/gCA | V/A | Unchanged |
| MUC4 | 3-195511138-AC-GT | GTt/ACt | V/T | Changed missense |
| MUC4 | 3-195514401-AGC-GGT | GcT/AcC | A/T | Unchanged |
| MUC4 | 3-195514776-CA-TG | gTG/gCA | V/A | Unchanged |
| UVSSA | 4-1377614-CA-TG | tCA/tTG | S/L | Unchanged |
| CRIPAK | 4-1388582-CA-TG | CAc/TGc | H/C | Changed missense |
| TACC3 | 4-1730298-CA-TG | gCA/gTG | A/V | Unchanged |
| ZFYVE28 | 4-2306051-CGA-TGG | TcG/CcA | S/P | Unchanged |
| RP11-503N18.3 | 4-2461887-CA-TG | CAc/TGc | H/C | Changed missense |
| RP11-503N18.3 | 4-2461888-AC-GT | cAC/cGT | H/R | Unchanged |
| NOP14 | 4-2951803-CA-TG | tTG/tCA | L/S | Unchanged |
| RGS12 | 4-3318412-CA-TG | tCA/tTG | S/L | Unchanged |
| DOK7 | 4-3494599-CA-TG | CAg/TGg | Q/W | Rescued nonsense |
| EVC | 4-5735119-AC-GT | gAC/gGT | D/G | Unchanged |
| WFS1 | 4-6303021-AC-GT | aAC/aGT | N/S | Unchanged |
| WFS1 | 4-6304086-CA-TG | tCA/tTG | S/L | Unchanged |
| PSAPL1 | 4-7435377-CAA-TAG | TtG/CtA | L | Unchanged |
| SH3TC1 | 4-8207082-CA-TG | cCA/cTG | P/L | Unchanged |
| SH3TC1 | 4-8242465-CA-TG | gCA/gTG | A/V | Unchanged |
| CPZ | 4-8602934-CA-TG | tCA/tTG | S/L | Unchanged |
| SLC2A9 | 4-10020693-CA-TG | TGg/CAg | W/Q | Rescued nonsense |
| BST1 | 4-15733403-AC-GT | ACa/GTa | T/V | Changed missense |
| FGFBP2 | 4-15964573-AC-GT | cGT/cAC | R/H | Unchanged |
| FAM184B | 4-17634213-AC-GT | GTc/ACc | V/T | Changed missense |
| SLC34A2 | 4-25678199-AC-GT | gAC/gGT | D/G | Unchanged |
| WDR19 | 4-39207317-CA-TG | tCA/tTG | S/L | Unchanged |
| PDS5A | 4-39921942-AGC-GGT | GcT/AcC | A/T | Unchanged |
| GRXCR1 | 4-42964918-AC-GT | ACt/GTt | T/V | Changed missense |
| SCFD2 | 4-54011525-CA-TG | tTG/tCA | L/S | Unchanged |
| TMEM165 | 4-56262372-CCA-TCG | CcA/TcG | P/S | Unchanged |
| KIAA1211 | 4-57182757-AC-GT | gAC/gGT | D/G | Unchanged |
| REST | 4-57797414-CA-TG | cCA/cTG | P/L | Unchanged |
| NOA1 | 4-57843320-AC-GT | tGT/tAC | C/Y | Unchanged |
| LPHN3 | 4-62845489-CA-TG | cCA/cTG | P/L | Unchanged |
| STAP1 | 4-68442967-CA-TG | aCA/aTG | T/M | Changed missense |
| UGT2B7 | 4-69972951-AC-GT | tAC/tGT | Y/C | Unchanged |
| UGT2B11 | 4-70079974-CA-TG | TGt/CAt | C/H | Changed missense |
| AFP | 4-74318329-CA-TG | aCA/aTG | T/M | Changed missense |
| MTHFD2L | 4-75040281-CA-GT | CAg/GTg | Q/V | Changed missense |
| FRAS1 | 4-79240063-AC-GT | gAC/gGT | D/G | Unchanged |
| FRAS1 | 4-79372929-AC-GT | cAC/cGT | H/R | Unchanged |
| PTPN13 | 4-87671749-CA-TG | tCA/tTG | S/L | Unchanged |
| PTPN13 | 4-87684030-CA-TG | cCA/cTG | P/L | Unchanged |
| DSPP | 4-88535868-AC-GT | aAC/aGT | N/S | Unchanged |
| DSPP | 4-88536437-AAC-GAT | AaC/GaT | N/D | Unchanged |
| DSPP | 4-88536899-AAC-GAT | AaC/GaT | N/D | Unchanged |
| DSPP | 4-88537400-AGC-GGT | AgC/GgT | S/G | Unchanged |
| DSPP | 4-88537418-AGC-GGT | AgC/GgT | S/G | Unchanged |
| DSPP | 4-88537556-AAC-GAT | AaC/GaT | N/D | Unchanged |
| GPRIN3 | 4-90169924-CA-TG | gTG/gCA | V/A | Unchanged |
| GRID2 | 4-94344031-AC-GT | tAC/tGT | Y/C | Unchanged |
| PDLIM5 | 4-95531416-CA-TG | gCA/gTG | A/V | Unchanged |
| UNC5C | 4-96106321-CA-TG | aTG/aCA | M/T | Partially changed missense |
| C4orf17 | 4-100443852-CA-TG | cCA/cTG | P/L | Unchanged |
| BANK1 | 4-102751028-CA-TG | aCA/aTG | T/M | Changed missense |
| BANK1 | 4-102751276-CTA-TTG | CtA/TtG | L | Unchanged |
| ARHGEF38 | 4-106534599-CA-TG | tCA/tTG | S/L | Unchanged |
| SEC24B | 4-110384789-CA-TG | cCA/cTG | P/L | Unchanged |
| EGF | 4-110901146-CA-TG | gCA/gTG | A/V | Unchanged |
| ALPK1 | 4-113350402-CA-TG | aCA/aTG | T/M | Changed missense |
| ALPK1 | 4-113359701-CGA-TGG | CgA/TgG | R/W | Rescued nonsense |
| ANK2 | 4-114277480-CA-TG | gCA/gTG | A/V | Unchanged |
| UGT8 | 4-115544713-CA-TG | cCA/cTG | P/L | Unchanged |
| QRFPR | 4-122250733-CA-TG | tTG/tCA | L/S | Unchanged |
| INTU | 4-128564998-CGA-TGG | CgA/TgG | R/W | Rescued nonsense |
| C4orf33 | 4-130032842-CA-TG | CAt/TGt | H/C | Changed missense |
| DCHS2 | 4-155295075-CA-TG | gTG/gCA | V/A | Unchanged |
| ANXA10 | 4-169098913-CA-TG | aCA/aTG | T/M | Changed missense |
| DDX60L | 4-169341434-CA-TG | TGt/CAt | C/H | Changed missense |
| ADAM29 | 4-175899095-CA-TG | CAg/TGg | Q/W | Rescued nonsense |
| NEIL3 | 4-178256958-AC-TG | gAC/gTG | D/V | Partially changed missense |
| TENM3 | 4-183715018-AC-GT | gAC/gGT | D/G | Unchanged |
| CDKN2AIP | 4-184367724-CA-TG | tCA/tTG | S/L | Unchanged |
| TRAPPC11 | 4-184600600-CA-TG | gCA/gTG | A/V | Unchanged |
| PRIMPOL | 4-185587164-CA-TG | CAg/TGg | Q/W | Rescued nonsense |
| SORBS2 | 4-186541301-AC-GT | cGT/cAC | R/H | Unchanged |
| FAM149A | 4-187078784-CA-TG | CAc/TGc | H/C | Changed missense |
| FAM149A | 4-187078785-AC-GT | cAC/cGT | H/R | Unchanged |
| FAT1 | 4-187629537-AC-GT | GTc/ACc | V/T | Changed missense |
| DUX4L4 | 4-191003179-AC-GT | ACg/GTg | T/V | Changed missense |
| PLEKHG4B | 5-181760-CGA-TGG | CgA/TgG | R/W | Rescued nonsense |
| SLC9A3 | 5-475103-CA-TG | TGc/CAc | C/H | Changed missense |
| SLC6A19 | 5-1216774-CA-TG | aCA/aTG | T/M | Changed missense |
| LPCAT1 | 5-1467003-CA-TG | aTG/aCA | M/T | Partially changed missense |
| SRD5A1 | 5-6651969-CA-TG | cCA/cTG | P/L | Unchanged |
| CDH12 | 5-21752056-ATC-GTT | GaT/AaC | D/N | Unchanged |
| PDZD2 | 5-32058114-CA-TG | tCA/tTG | S/L | Unchanged |
| AMACR | 5-33998882-CA-TG | tTG/tCA | L/S | Unchanged |
| TTC23L | 5-34840840-CA-TG | CAt/TGt | H/C | Changed missense |
| MROH2B | 5-41048532-AC-GT | cGT/cAC | R/H | Unchanged |
| MROH2B | 5-41061715-CA-TG | TGg/CAg | W/Q | Rescued nonsense |
| ZNF131 | 5-43175101-AGC-GGT | AgC/GgT | S/G | Unchanged |
| RP11-45H22.3 | 5-54253580-CA-TG | TGt/CAt | C/H | Changed missense |
| MCCC2 | 5-70945074-CA-TG | gCA/gTG | A/V | Unchanged |
| BTF3 | 5-72798844-CA-TG | aCA/aTG | T/M | Changed missense |
| ARHGEF28 | 5-73144844-CA-TG | tCA/tTG | S/L | Unchanged |
| CMYA5 | 5-79095299-AAC-GAT | AaC/GaT | N/D | Unchanged |
| ANKRD34B | 5-79855371-CA-TG | tTG/tCA | L/S | Unchanged |
| MSH3 | 5-80149980-CA-TG | CAg/TGg | Q/W | Rescued nonsense |
| ATG10 | 5-81549215-AC-GT | ACg/GTg | T/V | Changed missense |
| AC008394.1 | 5-86513973-CA-TG | TGc/CAc | C/H | Changed missense |
| TMEM161B | 5-87493524-AC-GT | GTa/ACa | V/T | Changed missense |
| DCP2 | 5-112328375-CTA-TTG | CtA/TtG | L | Unchanged |
| HSD17B4 | 5-118872183-AC-GT | ACa/GTa | T/V | Changed missense |
| IL13 | 5-131995963-CA-TG | CAg/TGg | Q/W | Rescued nonsense |
| SLC25A48 | 5-135178141-CA-TG | aCA/aTG | T/M | Changed missense |
| APBB3 | 5-139941227-CA-TG | TGc/CAc | C/H | Changed missense |
| PCDHA8 | 5-140222344-CGA-TGG | CgA/TgG | R/W | Rescued nonsense |
| PCDHA9 | 5-140229671-CTA-TTG | CtA/TtG | L | Unchanged |
| PCDHA10 | 5-140237221-CTA-TTG | CtA/TtG | L | Unchanged |
| PCDHA11 | 5-140250423-CTA-TTG | CtA/TiG | L | Unchanged |
| PCDHA12 | 5-140256624-CTA-TTG | CtA/TtG | L | Unchanged |
| PCDHB5 | 5-140516859-AGC-GGT | AgC/GgT | S/G | Unchanged |
| PCDHB10 | 5-140572667-AC-GT | aAC/aGT | N/S | Unchanged |
| PCDHB15 | 5-140627275-CA-TG | gCA/gTG | A/V | Unchanged |
| PCDHGB1 | 5-140731407-CA-TG | aCA/aTG | T/M | Changed missense |
| PCDH12 | 5-141337225-AGC-GGT | GcT/AcC | A/T | Unchanged |
| TCERG1 | 5-145838642-CA-TG | CAg/TGg | Q/W | Rescued nonsense |
| TCOF1 | 5-149755420-CA-TG | tCA/tTG | S/L | Unchanged |
| GM2A | 5-150639409-ATC-GTT | AtC/GtT | l/V | Unchanged |
| FAT2 | 5-150901612-CA-TG | tTG/tCA | L/S | Unchanged |
| MRPL22 | 5-154346258-CA-TG | tCA/tTG | S/L | Unchanged |
| HAVCR1 | 5-156479508-CA-TG | cTG/cCA | L/P | Unchanged |
| ATP10B | 5-16009749S-CA-TG | TGt/CAt | C/H | Changed missense |
| WWC1 | 5-167894863-CA-TG | CAc/TGc | H/C | Changed missense |
| SLIT3 | 5-168098235-ATC-GTT | GaT/AaC | D/N | Unchanged |
| SLIT3 | 5-168212951-AC-GT | GTc/ACc | V/T | Changed missense |
| DOCK2 | 5-169461545-ACC-GCT | AcC/GcT | T/A | Unchanged |
| PRELID1 | 5-176733114-CGA-TGG | CgA/TgG | R/W | Rescued nonsense |
| LMAN2 | 5-176778226-AC-GT | GTa/ACa | V/T | Changed missense |
| FAM153A | 5-177161891-ACC-GCT | GgT/AgC | G/S | Unchanged |
| ZFP2 | 5-178359134-CAA-T AG | CaA/TaG | Q/* | Unchanged |
| CSorf60 | 5-179069467-CA-TG | TGg/CAg | W/Q | Rescued nonsense |
| TBC1D9B | 5-179297352-AC-GT | tGT/tAC | C/Y | Unchanged |
| MGAT1 | 5-180218667-CA-TG | cTG/cCA | L/P | Unchanged |
| BTNL8 | 5-180338368-AC-GT | ACg/GTg | T/V | Changed missense |
| BTNL9 | 5-180472512-CA-TG | cCA/cTG | P/L | Unchanged |
| OR2V2 | 5-180582603-CA-TG | CAc/TGc | H/C | Changed missense |
| NQO2 | 6-3015789-CA-TG | cCA/cTG | P/L | Unchanged |
| RIPK1 | 6-3105857-AC-TG | aAC/aTG | N/M | Changed missense |
| PPP1R3G | 6-5086069-CA-TG | gCA/gTG | A/V | Unchanged |
| RREB1 | 6-7231769-CA-TG | cCA/cTG | P/L | Unchanged |
| DSP | 6-7556048-CA-TG | CAg/TGg | Q/W | Rescued nonsense |
| HIVEP1 | 6-12122174-AC-GT | ACg/GTg | T/V | Changed missense |
| NUP153 | 6-17688691-ATC-GTT | GaT/AaC | D/N | Unchanged |
| LRRC16A | 6-25435757-CA-TG | tCA/tTG | S/L | Unchanged |
| HIST1H2BA | 6-25727263-ATC-GTT | AtC/GtT | I/V | Unchanged |
| HIST1H1A | 6-26017673-CAA-TAG | TtG/CtA | L | Unchanged |
| BTN3A1 | 6-26406394-AAC-GAT | AaC/GaT | N/D | Unchanged |
| OR2J2 | 6-29141771-CA-TG | tCA/tTG | S/L | Unchanged |
| OR2J2 | 6-29142064-ACC-GCT | AcC/GcT | T/A | Unchanged |
| UBD | 6-29523676-CA-GT | TGt/ACt | C/T | Changed missense |
| UBD | 6-29524002-CA-TG | tTG/tCA | L/S | Unchanged |
| MUC21 | 6-30954919-AC-GT | ACc/GTc | T/V | Changed missense |
| MUC21 | 6-30955009-AC-GT | ACc/GTc | T/V | Changed missense |
| MUC22 | 6-30993590-ATC-GTT | AtC/GtT | I/V | Unchanged |
| MUC22 | 6-30993957-CA-TG | gCA/gTG | A/V | Unchanged |
| MUC22 | 6-31002452-AAC-GAT | AaC/GaT | N/D | Unchanged |
| C6orf15 | 6-31079893-CA-TG | gTG/gCA | V/A | Unchanged |
| SLC44A4 | 6-31839308-AC-GT | GTt/ACt | V/T | Changed missense |
| CFB | 6-31914933-CGA-TGG | CgA/TgG | R/W | Rescued nonsense |
| CYP21A2 | 6-32006871-AC-GT | tAC/tGT | Y/C | Unchanged |
| RNF5 | 6-32148029-CCA-TCG | CcA/TcG | P/S | Unchanged |
| HLA-DRA | 6-32410986-CA-TG | aCA/aTG | T/M | Changed missense |
| HLA-DRB5 | 6-32497942-CA-TG | aTG/aCA | M/T | Partially changed missense |
| HLA-DQA1 | 6-32610494-CA-TG | CAa/TGa | Q/* | Unchanged |
| HLA-DQB1 | 6-32632687-ATC-GTT | GaT/AaC | D/N | Unchanged |
| HLA-DQB1 | 6-32632777-CA-TG | gTG/gCA | V/A | Unchanged |
| PSMB9 | 6-32826232-CA-TG | gCA/gTG | A/V | Unchanged |
| HLA-DPA1 | 6-33036950-CA-TG | cTG/cCA | L/P | Unchanged |
| tTPR3 | 6-33636906-AC-GT | cAC/cGT | H/R | Unchanged |
| UHRF1BP1 | 6-34824635-CA-TG | CAg/TGg | Q/W | Rescued nonsense |
| ZNF76 | 6-35260502-AC-GT | cAC/cGT | H/R | Unchanged |
| KtF6 | 6-39325077-CA-TG | TGg/CAg | W/Q | Rescued nonsense |
| FRS3 | 6-41739191-AC-TG | gGT/gCA | G/A | Unchanged |
| C6orf132 | 6-42075068-CA-TG | tTG/tCA | L/S | Unchanged |
| GPR111 | 6-47624298-CA-TG | CAt/TGt | H/C | Changed missense |
| GPR115 | 6-47680256-CA-TG | aCA/aTG | T/M | Changed missense |
| GPR115 | 6-47682671-ATC-GTT | AtC/GtT | I/V | Unchanged |
| FAM83B | 6-54806378-CA-TG | cCA/cTG | P/L | Unchanged |
| C6orf57 | 6-71289188-CA-TG | CAg/TGg | Q/W | Rescued nonsense |
| RtMS1 | 6-72967899-CA-TG | CAt/TGt | H/C | Changed missense |
| DDX43 | 6-74125887-CA-TG | CAg/TGg | Q/W | Rescued nonsense |
| CD109 | 6-74521946-AC-GT | ACg/GTg | T/V | Changed missense |
| FILIP1 | 6-76023772-AC-GT | aGT/aAC | S/N | Unchanged |
| SENP6 | 6-76369052-CGA-TGG | CgA/TgG | R/W | Rescued nonsense |
| IRAK1BP1 | 6-79595096-CA-TG | gCA/gTG | A/V | Unchanged |
| NT5E | 6-86199233-AC-GT | ACg/GTg | T/V | Changed missense |
| COL10A1 | 6-116446575-CA-TG | aTG/aCA | M/T | Partially changed missense |
| TBC1D32 | 6-121625944-CA-TG | tTG/tCA | L/S | Unchanged |
| PTPRK | 6-128403745-AAC-GAT | GtT/AtC | V/I | Unchanged |
| ENPP3 | 6-131995312-CA-TG | cCA/cTG | P/L | Unchanged |
| VNN2 | 6-133072650-AC-GT | gGT/gAC | G/D | Unchanged |
| IL20RA | 6-137325846-AC-GT | GTa/ACa | V/T | Changed missense |
| RP3-422G23.4 | 6-138703761-CA-TG | cCA/cTG | P/L | Unchanged |
| GPR126 | 6-142758630-CA-TG | CAg/TGg | Q/W | Rescued nonsense |
| HIVEP2 | 6-143091262-CA-TG | cTG/cCA | L/P | Unchanged |
| EPM2A | 6-145948739-AC-GT | GTg/ACg | V/T | Changed missense |
| ADGB | 6-147136243-CA-TG | CAc/TGc | H/C | Changed missense |
| SYNE1 | 6-152464839-AC-GT | cGT/cAC | R/H | Unchanged |
| MTRF1L | 6-153323806-AAC-GAT | GtT/AtC | V/I | Unchanged |
| TULR4 | 6-158735085-AGC-GGT | AgC/GgT | S/G | Unchanged |
| SYTL3 | 6-159184461-CA-TG | tCA/tTG | S/L | Unchanged |
| IGF2R | 6-160453977-CA-TG | tCA/tTG | S/L | Unchanged |
| IGF2R | 6-160505207-AC-GT | aAC/aGT | N/S | Unchanged |
| LPA | 6-160969628-CA-TG | aTG/aCA | M/T | Partially changed missense |
| WDR27 | 6-170070722-CA-TG | cTG/cCA | L/P | Unchanged |
| ERMARD | 6-170155480-CGA-TGG | CgA/TgG | R/W | Rescued nonsense |
| FAM120B | 6-170627803-AC-GT | gAC/gGT | D/G | Unchanged |
| FAM120B | 6-170627835-AC-GT | ACa/GTa | T/V | Changed missense |
| FAM120B | 6-170627836-CA-TG | aCA/aTG | T/M | Changed missense |
| AC145676.2 | 7-228658-CA-TG | TGt/CAt | C/H | Changed missense |
| FAM20C | 7-299881-AAC-GAT | AaC/GaT | N/D | Unchanged |
| SUN1 | 7-881668-CA-TG | CAc/TGc | H/C | Changed missense |
| SUN1 | 7-897491-CA-TG | aCA/aTG | T/M | Changed missense |
| GPR146 | 7-1097894-CA-TG | cCA/cTG | P/L | Unchanged |
| MICALL2 | 7-1484572-AC-GT | gGT/gAC | G/D | Unchanged |
| SDK1 | 7-4213974-CA-TG | CAt/TGt | H/C | Changed missense |
| ZNF12 | 7-6731865-CA-TG | aTG/aCA | M/T | Partially changed missense |
| MIOS | 7-7646686-CA-TG | aCA/aTG | T/M | Changed missense |
| DNAH11 | 7-21603885-CA-TG | cCA/cTG | P/L | Unchanged |
| DNAH11 | 7-21628196-CA-TG | CAg/TGg | Q/W | Rescued nonsense |
| DNAH11 | 7-21630981-CA-TG | gCA/gTG | A/V | Unchanged |
| FAM188B | 7-30915260-AAC-GAT | AaC/GaT | N/D | Unchanged |
| AQP1 | 7-30915260-AAC-GAT | AaC/GaT | N/D | Unchanged |
| FAM138B | 7-30915261-AC-GT | aAC/aGT | N/S | Unchanged |
| AQP1 | 7-30915261-AC-GT | aAC/aGT | N/S | Unchanged |
| CCDC129 | 7-31618007-CA-TG | CAt/TGt | H/C | Changed missense |
| AVL9 | 7-32613019-CA-TG | aCA/aTG | T/M | Changed missense |
| AEBP1 | 7-44148552-CA-TG | aCA/aTG | T/M | Changed missense |
| NACAD | 7-45124285-CA-TG | gTG/gCA | V/A | Unchanged |
| ABCA13 | 7-48285165-CAA-TAG | CaA/TaG | Q/* | Unchanged |
| C7orf72 | 7-50173777-ACC-GCT | AcC/GcT | T/A | Unchanged |
| LANCL2 | 7-55459562-CA-TG | aCA/aTG | T/M | Changed missense |
| ZNF479 | 7-57193783-CA-TG | cTG/cCA | L/P | Unchanged |
| GUSB | 7-65425893-CA-TG | cTG/cCA | L/P | Unchanged |
| TYW1 | 7-66648155-AAC-GAT | AaC/GaT | N/D | Unchanged |
| TYW1 | 7-66660241-CA-TG | CAc/TGc | H/C | Changed missense |
| TYW1 | 7-66660242-AC-GT | cAC/cGT | H/R | Unchanged |
| TRIM74 | 7-72436651-CA-TG | TGg/CAg | W/Q | Rescued nonsense |
| TRIM74 | 7-72436665-CA-TG | cTG/cCA | L/P | Unchanged |
| TRIM50 | 7-72738748-CA-TG | TGg/CAg | W/Q | Rescued nonsense |
| TRIM50 | 7-72738762-CA-TG | cTG/cCA | L/P | Unchanged |
| STX1A | 7-73122923-ATC-GTT | GaT/AaC | D/N | Unchanged |
| WBSCR28 | 7-73280018-ACC-GCT | AcC/GcT | T/A | Unchanged |
| TRIM73 | 7-75028468-CA-TG | cCA/cTG | P/L | Unchanged |
| RHBDD2 | 7-75513061-CA-TG | gCA/gTG | A/V | Unchanged |
| DTX2 | 7-76131645-AC-GT | ACa/GTa | T/V | Changed missense |
| DTX2 | 7-76131705-CA-TG | CAc/TGc | H/C | Changed missense |
| DMTF1 | 7-86800350-CA-TG | gCA/gTG | A/V | Unchanged |
| ABCB1 | 7-87179601-ACC-GCT | GgT/AgC | G/S | Unchanged |
| BAIAP2L1 | 7-97933601-CA-TG | tTG/tCA | L/S | Unchanged |
| PVRIG | 7-99817859-AAC-GAT | AaC/GaT | N/D | Unchanged |
| FBXO24 | 7-100198385-AC-GT | ACg/GTg | T/V | Changed missense |
| MUC12 | 7-100634074-AC-GT | cAC/cGT | H/R | Unchanged |
| MUC12 | 7-100636852-CA-TG | aCA/aTG | T/M | Changed missense |
| MUC12 | 7-100638855-CA-TG | CAc/TGc | H/C | Changed missense |
| MUC12 | 7-100646746-CA-TG | aCA/aTG | T/M | Changed missense |
| MUC17 | 7-100677585-CA-TG | tCA/tTG | S/L | Unchanged |
| MUC17 | 7-100677641-AC-GT | ACg/GTg | T/V | Changed missense |
| MUC17 | 7-100682274-CA-TG | cCA/cTG | P/L | Unchanged |
| MUC17 | 7-100684749-CA-TG | cCA/cTG | P/L | Unchanged |
| CUX1 | 7-101844850-CA-TG | cCA/cTG | P/L | Unchanged |
| NAPEPLD | 7-102743891-ATC-GTT | GaT/AaC | D/N | Unchanged |
| PIK3CG | 7-106513009-AGC-GGT | AgC/GgT | S/G | Unchanged |
| GPR85 | 7-112724525-ACC-GCT | GgT/AgC | G/S | Unchanged |
| ST7-OT4 | 7-116596696-CA-TG | CAt/TGt | H/C | Changed missense |
| CCDC136 | 7-128455767-CAA-GAT | CaA/GaT | Q/D | Changed missense |
| CPA5 | 7-129986336-ACC-GCT | AcC/GcT | T/A | Unchanged |
| PLXNA4 | 7-131815310-ATC-GTT | GaT/AaC | D/N | Unchanged |
| PLXNA4 | 7-131883385-AC-TG | GTg/CAg | V/Q | Changed missense |
| AKR1B10 | 7-134221825-CA-TG | aCA/aTG | T/M | Changed missense |
| CREB3L2 | 7-137600689-AC-GT | GTt/ACt | V/T | Changed missense |
| ATP6V0A4 | 7-138417790-CA-TG | aTG/aCA | M/T | Partially changed missense |
| ATP6V0A4 | 7-138455987-CA-TG | gTG/gCA | V/A | Unchanged |
| TRBV6-9 | 7-142104527-CA-TG | gTG/gCA | V/A | Unchanged |
| TRBV7-4 | 7-142176377-CA-TG | TGc/CAc | C/H | Changed missense |
| OR9A2 | 7-142723453-AC-GT | GTg/ACg | V/T | Changed missense |
| CLCN1 | 7-143042836-AC-GT | gAC/gGT | D/G | Unchanged |
| EPHA1 | 7-143097099-CA-TG | gTG/gCA | V/A | Unchanged |
| OR2F1 | 7-143657800-CA-TG | aCA/aTG | T/M | Changed missense |
| OR2A14 | 7-143826695-AGC-TGG | AgC/TgG | S/W | Changed missense |
| KRBA1 | 7-149421772-CA-TG | CAt/TGt | H/C | Changed missense |
| ZNF775 | 7-150094851-AC-GT | ACg/GTg | T/V | Changed missense |
| GIMAP2 | 7-150389856-AC-GT | cAC/cGT | H/R | Unchanged |
| TMEM176A | 7-150501516-ACC-GCT | AcC/GcT | T/A | Unchanged |
| AOC1 | 7-150555914-CA-TG | cCA/cTG | P/L | Unchanged |
| SLC4A2 | 7-150771792-AC-GT | ACt/GTt | T/V | Changed missense |
| DPP6 | 7-154667627-CA-TG | cCA/cTG | P/L | Unchanged |
| AC021218.2 | 7-155755825-CA-TG | tCA/tTG | S/L | Unchanged |
| RNF32 | 7-156469189-CA-TG | gCA/gTG | A/V | Unchanged |
| WDR60 | 7-158672618-CA-TG | CAa/TGa | Q/* | Unchanged |
| ARHGEF10 | 8-1900910-AC-GT | cAC/cGT | H/R | Unchanged |
| TNKS | 8-9564436-CA-TG | aCA/aTG | T/M | Changed missense |
| NEIL2 | 8-11640783-CA-TG | cCA/cTG | P/L | Unchanged |
| KIAA1456 | 8-12878636-CA-TG | CAc/TGc | H/C | Changed missense |
| KIAA1456 | 8-12878828-AGC-GGT | AgC/GgT | S/G | Unchanged |
| DLC1 | 8-12957474-AC-GT | GTg/ACg | V/T | Changed missense |
| MTMR7 | 8-17270787-AC-GT | cGT/cAC | R/H | Unchanged |
| MTUS1 | 8-17612874-CA-TG | TGt/CAt | C/H | Changed missense |
| PCM1 | 8-17796382-AC-GT | aAC/aGT | N/S | Unchanged |
| PDLIM2 | 8-22442660-CA-TG | gCA/gTG | A/V | Unchanged |
| TNFRSF10D | 8-23001987-CA-TG | tTG/tCA | L/S | Unchanged |
| R3HCC1 | 8-23148929-CA-TG | aCA/aTG | T/M | Changed missense |
| R3HCC1 | 8-23152333-CA-TG | aCA/aTG | T/M | Changed missense |
| DOCK5 | 8-25203021-CA-TG | aCA/aTG | T/M | Changed missense |
| DOCK5 | 8-25267621-CA-TG | cCA/cTG | P/L | Unchanged |
| ADRA1A | 8-26628027-CA-TG | TGc/CAc | C/H | Changed missense |
| MAK16 | 8-33356073-CA-TG | CAg/TGg | Q/W | Rescued nonsense |
| PROSC | 8-37623819-ATC-GTT | AtC/GtT | I/V | Unchanged |
| DDHD2 | 8-38095661-AC-GT | ACg/GTg | T/V | Changed missense |
| PREX2 | 8-69143588-CA-TG | cCA/cTG | P/L | Unchanged |
| SBSPON | 8-73982160-CA-TG | TGg/CAg | W/Q | Rescued nonsense |
| ZFHX4 | 8-77765864-AC-GT | tAC/tGT | Y/C | Unchanged |
| PEX2 | 8-77895864-CA-TG | TGt/CAt | C/H | Changed missense |
| RSPO2 | 8-108970366-CA-TG | cTG/cCA | L/P | Unchanged |
| PKHD1L1 | 8-110416812-AC-GT | tAC/tGT | Y/C | Unchanged |
| PKHD1L1 | 8-110437383-CA-TG | CAt/TGt | H/C | Changed missense |
| TBC1D31 | 8-124121797-CA-TG | gCA/gTG | A/V | Unchanged |
| HHLA1 | 8-133089974-CA-TG | tTG/tCA | L/S | Unchanged |
| ZFAT | 8-135612745-ATC-GTT | GaT/AaC | D/N | Unchanged |
| ZFAT | 8-135614705-AC-GT | cGT/cAC | R/H | Unchanged |
| SLC45A4 | 8-142222374-ACC-GCT | GgT/AgC | G/S | Unchanged |
| CYP11B2 | 8-143994265-CA-TG | gTG/gCA | V/A | Unchanged |
| MROH6 | 8-144649550-AC-GT | cGT/cAC | R/H | Unchanged |
| MROH6 | 8-144654254-CA-TG | gTG/gCA | V/A | Unchanged |
| EEF1D | 8-144671223-AGC-GGT | GcT/AcC | A/T | Unchanged |
| FAM83H | 8-144808955-AC-TG | aGT/aCA | S/T | Partially changed missense |
| PLEC | 8-144990528-AGC-GGT | GcT/AcC | A/T | Unchanged |
| PLEC | 8-145011204-AGC-GGT | GcT/AcC | A/T | Unchanged |
| PARP10 | 8-145057867-CA-TG | gTG/gCA | V/A | Unchanged |
| SLC39A4 | 8-145640410-CA-TG | TGg/CAg | W/Q | Rescued nonsense |
| KIFC2 | 8-145693719-CA-TG | gCA/gTG | A/V | Unchanged |
| KCNV2 | 9-2718497-CA-TG | cCA/cTG | P/L | Unchanged |
| GLIS3 | 9-4118206-CGA-TGG | TcG/CcA | S/P | Unchanged |
| JAK2 | 9-5050705-AC-GT | cAC/cGT | H/R | Unchanged |
| ADAMTSL1 | 9-18681820-CA-TG | aCA/aTG | T/M | Changed missense |
| TEK | 9-27202869-CA-TG | tCA/tTG | S/L | Unchanged |
| RECK | 9-36105263-CA-TG | cCA/cTG | P/L | Unchanged |
| GRHPR | 9-37429813-CA-TG | gCA/gTG | A/V | Unchanged |
| SPATA31A3 | 9-40702845-AC-GT | ACc/GTc | T/V | Changed missense |
| OSTF1 | 9-77752508-ATC-GTT | AtC/GtT | I/V | Unchanged |
| PRUNE2 | 9-79322673-CA-TG | TGt/CAt | C/H | Changed missense |
| SPATA31D1 | 9-84607216-AAC-GAT | AaC/GaT | N/D | Unchanged |
| OMD | 9-95179178-AC-GT | aGT/aAC | S/N | Unchanged |
| FGD3 | 9-95780464-CA-TG | aCA/aTG | T/M | Changed missense |
| C9orf156 | 9-100684755-CGA-TGG | TcG/CcA | S/P | Unchanged |
| TBC1D2 | 9-100972949-CA-TG | cTG/cCA | L/P | Unchanged |
| TBC1D2 | 9-100995720-CA-TG | tTG/tCA | L/S | Unchanged |
| INVS | 9-103015206-CTA-TTG | CtA/TtG | L | Unchanged |
| ZNF189 | 9-104162227-AC-GT | gAC/gGT | D/G | Unchanged |
| OR13C5 | 9-107360825-CA-TG | aTG/aCA | M/T | Partially changed missense |
| RAD23B | 9-110084388-CA-GT | aCA/aGT | T/S | Changed missense |
| PALM2-AKAP2 | 9-112900784-CA-TG | tCA/tTG | S/L | Unchanged |
| AKAP2 | 9-112900784-CA-TG | tCA/tTG | S/L | Unchanged |
| RGS3 | 9-116346286-AC-GT | tAC/tGT | Y/C | Unchanged |
| DFNB31 | 9-117166245-CA-TG | gTG/gCA | V/A | Unchanged |
| DFNB31 | 9-117169032-CA-TG | aTG/aCA | M/T | Partially changed missense |
| DFNB31 | 9-117186677-AC-GT | gGT/gAC | G/D | Unchanged |
| OR1Q1 | 9-125377086-CA-TG | CAa/TGa | Q/* | Unchanged |
| OR1B1 | 9-125391407-CAA-T AG | TtG/CtA | L | Unchanged |
| WDR38 | 9-127618775-CA-TG | CAc/TGc | H/C | Changed missense |
| RABEPK | 9-127996045-CA-TG | cCA/cTG | P/L | Unchanged |
| ZNF79 | 9-130191186-CA-TG | aCA/aTG | T/M | Changed missense |
| LRSAM1 | 9-130242166-AAC-GAT | AaC/GaT | N/D | Unchanged |
| AL359091.2 | 9-131063361-CA-TG | CAt/TGt | H/C | Changed missense |
| ODF2 | 9-131235320-CA-TG | aCA/aTG | T/M | Changed missense |
| SPTAN1 | 9-131375699-CTA-TTG | CtA/TtG | L | Unchanged |
| ASS1 | 9-133346225-AC-GT | cAC/cGT | H/R | Unchanged |
| LAMC3 | 9-133911597-CA-TG | gCA/gTG | A/V | Unchanged |
| NTNG2 | 9-135105964-AC-GT | ACt/GTt | T/V | Changed missense |
| DBH | 9-136518096-CA-TG | aCA/aTG | T/M | Changed missense |
| AL390778.1 | 9-138150587-CA-TG | cTG/cCA | L/P | Unchanged |
| AL390778.1 | 9-138151156-CA-TG | tTG/tCA | L/S | Unchanged |
| PPP1R26 | 9-138378856-AAC-GAT | AaC/GaT | N/D | Unchanged |
| SEC16A | 9-139369062-CA-TG | tTG/tCA | L/S | Unchanged |
| C8G | 9-139840543-AC-GT | gAC/gGT | D/G | Unchanged |
| C9orf141 | 9-139864340-CA-TG | CAg/TGg | Q/W | Rescued nonsense |
| ENTPD8 | 9-140332477-CA-TG | cTG/cCA | L/P | Unchanged |
| PNPLA7 | 9-140357971-CA-TG | TGg/CAg | W/Q | Rescued nonsense |
| PNPLA7 | 9-140396120-CA-TG | gTG/gCA | V/A | Unchanged |
| EHMT1 | 9-140611120-CA-GT | gCA/gGT | A/G | Unchanged |
| ASMTL | X-1546876-AC-GT | cGT/cAC | R/H | Unchanged |
| GYG2 | X-2779569-CA-TG | CAc/TGc | H/C | Changed missense |
| GYG2 | X-2779570-AC-GT | cAC/cGT | H/R | Unchanged |
| MXRA5 | X-3241284-AC-GT | aGT/aAC | S/N | Unchanged |
| PRKX | X-3631166-CA-TG | gTG/gCA | V/A | Unchanged |
| VCX2 | X-8138181-CA-TG | cTG/cCA | L/P | Unchanged |
| SHROOM2 | X-9859097-CA-TG | cCA/cTG | P/L | Unchanged |
| MAGEB16 | X-35820794-CA-TG | CAc/TGc | H/C | Changed missense |
| ZNF157 | X-47272530-CA-TG | aCA/aTG | T/M | Changed missense |
| XAGE5 | X-52842212-CA-TG | CAg/TGg | Q/W | Rescued nonsense |
| TSPYL2 | X-53115057-AAC-GAT | AaC/GaT | N/D | Unchanged |
| MAGED2 | X-54836360-CA-TG | tCA/tTG | S/L | Unchanged |
| HEPH | X-65427123-CA-TG | aCA/aTG | T/M | Changed missense |
| KIF4A | X-69516919-AC-TG | ACt/TGt | T/C | Changed missense |
| FAM46D | X-79698592-AC-TG | gAC/gTG | D/V | Partially changed missense |
| RBMXL3 | X-114425236-AC-GT | tAC/tGT | Y/C | Unchanged |
| LONRF3 | X-118148228-CA-TG | gCA/gTG | A/V | Unchanged |
| CT47B1 | X-120007783-CA-TG | gTG/gCA | V/A | Unchanged |
| CCDC160 | X-133379551-CTA-TTG | CtA/TtG | L | Unchanged |
| SPANXD | X-140785829-CGA-TGG | TcG/CcA | S/P | Unchanged |
| SPANXN3 | X-142596981-AC-GT | GTa/ACa | V/T | Changed missense |
| MAGEA8 | X-149013726-CA-TG | gCA/gTG | A/V | Unchanged |
| MAGEA4 | X-151092219-CA-TG | gCA/gTG | A/V | Unchanged |
| CSAG1 | X-151908844-AC-TG | tAC/tTG | Y/L | Rescued nonsense |
| ATP6AP1 | X-153657081-CGA-TGG | CgA/TgG | R/W | Rescued nonsense |
| PLXNA3 | X-153694819-CA-TG | aCA/aTG | T/M | Changed missense |

TABLE I
Specific targetable MNV mutations from the gnomAD database that can
be created with SPACE using Cas9 proteins with NGG PAM recognition.

| Gene_name | MNV | MNV_codons | MNV_AAs | MNV_category |
|---|---|---|---|---|
| SAMD11 | 1-877831-TG-CA | TGg/CAg | W/Q | Rescued nonsense |
| NOC2L | 1-888657-GAT-AAC | AtC/GtT | I/V | Unchanged |
| C1orf170 | 1-914940-TG-CA | gCA/gTG | A/V | Unchanged |
| CPSF3L | 1-1247494-TG-CA | cCA/cTG | P/L | Unchanged |
| TAS1R3 | 1-1269554-TG-CA | TGc/CAc | C/H | Changed missense |
| DVL1 | 1-1277533-TG-CA | cCA/cTG | P/L | Unchanged |
| MIB2 | 1-1558792-TG-CA | aTG/aCA | M/T | Partially changed missense |
| PEX10 | 1-2340200-TG-CA | aCA/aTG | T/M | Changed missense |
| PERS | 1-7870047-GT-AC | aGT/aAC | S/N | Unchanged |
| GPR157 | 1-9171348-TG-AC | CAc/GTc | H/V | Changed missense |
| C1orf127 | 1-11008695-TG-CA | cCA/cTG | P/L | Unchanged |
| C1orf167 | 1-11836628-TG-CA | TGg/CAg | W/Q | Rescued nonsense |
| C1orf167 | 1-11839998-TG-CA | TGg/CAg | W/Q | Rescued nonsense |
| TNFRSF1B | 1-12252927-GT-AC | GTg/ACg | V/T | Changed missense |
| DHRS3 | 1-12638964-TG-CA | cCA/cTG | P/L | Unchanged |
| AADACL3 | 1-12779558-GAT-AAC | GaT/AaC | D/N | Unchanged |
| PRAMEF10 | 1-12954806-TG-CA | aCA/aTG | T/M | Changed missense |
| PRAMEF26 | 1-13219066-TG-CA | CAc/TGc | H/C | Changed missense |
| PRAMEF13 | 1-13448351-TG-CA | CAc/TGc | H/C | Changed missense |
| CLCNKB | 1-16380196-TG-CA | aTG/aCA | M/T | Partially changed missense |
| PADI4 | 1-17657616-TG-CA | gTG/gCA | V/A | Unchanged |
| TAS1R2 | 1-19181391-GAT-AAC | AtC/GtT | I/V | Unchanged |
| VWA5B1 | 1-20644156-TG-CA | TGt/CAt | C/H | Changed missense |
| HSPG2 | 1-22149935-TG-CA | tCA/tTG | S/L | Unchanged |
| SFN | 1-27189854-GT-CA | GTg/CAg | V/Q | Changed missense |
| C1orf172 | 1-27278573-TG-CA | CAg/TGg | Q/W | Rescued nonsense |
| MAP3K6 | 1-27684006-TG-CA | CAc/TGc | H/C | Changed missense |
| WASF2 | 1-27736478-TG-CA | cCA/cTG | P/L | Unchanged |
| THEMIS2 | 1-28209360-GTT-ATC | GtT/AtC | V/I | Unchanged |
| ZFP69B | 1-40923018-TG-CA | TGt/CAt | C/H | Changed missense |
| HIVEP3 | 1-42048745-TG-CA | CAc/TGc | H/C | Changed missense |
| PTPRF | 1-44071220-TG-CA | gTG/gCA | V/A | Unchanged |
| STIL | 1-47726087-TG-CA | CAt/TGt | H/C | Changed missense |
| SLC1A7 | 1-53553754-TG-CA | CAg/TGg | Q/W | Rescued nonsense |
| CACHD1 | 1-65113568-TG-CA | aTG/aCA | M/T | Partially changed missense |
| TGFBR3 | 1-92262874-TG-CA | gCA/gTG | A/V | Unchanged |
| RWDD3 | 1-95709821-TG-CA | gTG/gCA | V/A | Unchanged |
| GSTM5 | 1-110256333-GT-AC | GTt/ACt | V/T | Changed missense |
| GSTM5 | 1-110256333-GTT-ATC | GtT/AtC | V/I | Unchanged |
| EPS8L3 | 1-110295772-TG-CA | tCA/tTG | S/L | Unchanged |
| IGSF3 | 1-117156584-TG-CA | CAg/TGg | Q/W | Rescued nonsense |
| NBPF14 | 1-148004782-GCT-ACC | AgC/GgT | S/G | Unchanged |
| HRNR | 1-152191750-TG-CA | tCA/tTG | S/L | Unchanged |
| FLG | 1-152275725-TCG-CCA | CgA/TgG | R/W | Rescued nonsense |
| FLG | 1-152276827-TG-CA | CAg/TGg | Q/W | Rescued nonsense |
| FLG | 1-152285413-TG-CA | CAg/TGg | Q/W | Rescued nonsense |
| FLG | 1-152285512-TG-CA | CAg/TGg | Q/W | Rescued nonsense |
| IVL | 1-152882851-TG-CA | cTG/cCA | L/P | Unchanged |
| SPRR2D | 1-153012764-GT-AC | ACg/GTg | T/V | Changed missense |
| LOR | 1-153233519-GGT-AGC | GgT/AgC | G/S | Unchanged |
| RXFP4 | 1-155912486-TG-CA | tTG/tCA | L/S | Unchanged |
| MEF2D | 1-156450740-GT-AC | aAC/aGT | N/S | Unchanged |
| IQGAP3 | 1-156532977-TTG-CTA | CaA/TaG | Q/* | Unchanged |
| OR6N1 | 1-158735691-TG-CA | CAg/TGg | Q/W | Rescued nonsense |
| C1orf204 | 1-159824966-TG-AC | CAa/GTa | Q/V | Changed missense |
| DCAF6 | 1-168013850-TG-CA | gTG/gCA | V/A | Unchanged |
| SELP | 1-169566308-GT-AC | aAC/aGT | N/S | Unchanged |
| TNN | 1-175086123-TG-CA | aTG/aCA | M/T | Partially changed missense |
| SEC16B | 1-177929987-TG-CA | CAt/TGt | H/C | Changed missense |
| LAMC1 | 1-183105533-GT-AC | cGT/cAC | R/H | Unchanged |
| RNPEP | 1-201970560-GTT-ATC | GtT/AtC | V/I | Unchanged |
| PLXNA2 | 1-208207839-GT-AC | tAC/tGT | Y/C | Unchanged |
| OR2T33 | 1-248436965-TG-CA | CAc/TGc | H/C | Changed missense |
| OR2T12 | 1-248458729-TG-CA | CAc/TGc | H/C | Changed missense |
| OR2T29 | 1-248722722-TG-CA | CAa/TGa | Q/* | Unchanged |
| OR2T27 | 1-248813292-TG-CA | aCA/aTG | T/M | Changed missense |
| PITRM1 | 10-3180227-TG-CA | CAa/TGa | Q/* | Unchanged |
| ECHDC3 | 10-11797434-GTT-ATC | GtT/AtC | V/I | Unchanged |
| CUBN | 10-16882518-TG-CA | aCA/aTG | T/M | Changed missense |
| C10ORF68 | 10-33137551-TG-CA | aTG/aCA | M/T | Partially changed missense |
| GPRIN2 | 10-47000004-TG-CA | gTG/gCA | V/A | Unchanged |
| FRMPD2 | 10-49380999-TG-CA | tCA/tTG | S/L | Unchanged |
| WDFY4 | 10-50109902-GT-AC | aGT/aAC | S/N | Unchanged |
| LRRC18 | 10-50121784-TG-CA | aCA/aTG | T/M | Changed missense |
| CHAT | 10-50856652-GT-AC | GTg/ACg | V/T | Changed missense |
| PCDH15 | 10-55996608-TG-CA | cCA/cTG | P/L | Unchanged |
| HKDC1 | 10-71018660-TG-CA | TGg/CAg | W/Q | Rescued nonsense |
| HKDC1 | 10-71018660-TGG-CGA | TgG/CgA | W/R | Rescued nonsense |
| PLAU | 10-75673101-TG-CA | cTG/cCA | L/P | Unchanged |
| KAT6B | 10-76789077-GT-AC | GTa/ACa | V/T | Changed missense |
| DLG5 | 10-79616605-TG-CA | CAg/TGg | Q/W | Rescued nonsense |
| NUTM2B | 10-81471741-TG-CA | TGg/CAg | W/Q | Rescued nonsense |
| LRIT1 | 10-85992511-TG-CA | cCA/cTG | P/L | Unchanged |
| CEP55 | 10-95276718-TG-CA | TGt/CAt | C/H | Changed missense |
| SORBS1 | 10-97174351-GT-AC | ACg/GTg | T/V | Changed missense |
| DNMBP | 10-101645498-TG-CA | cCA/cTG | P/L | Unchanged |
| CALHM1 | 10-105218359-TG-CA | gCA/gTG | A/V | Unchanged |
| NHLRC2 | 10-115636469-TG-CA | aTG/aCA | M/T | Partiaiiy changed missense |
| BAG3 | 10-121429633-TG-CA | TGt/CAt | C/H | Changed missense |
| PLEKHA1 | 10-124183731-GT-AC | cGT/cAC | R/H | Unchanged |
| DMBT1 | 10-124345741-TG-CA | tTG/tCA | L/S | Unchanged |
| C10orf90 | 10-128202435-TG-CA | gCA/gTG | A/V | Unchanged |
| GPR123 | 10-134910553-GT-CA | GTc/CAc | V/H | Changed missense |
| MUC2 | 11-1081074-TG-CA | gTG/gCA | V/A | Unchanged |
| MUC2 | 11-1083291-GAT-AAC | GaT/AaC | D/N | Unchanged |
| MUC5B | 11-1265936-TG-CA | cTG/cCA | L/P | Unchanged |
| KRTAP5-5 | 11-1651161-TG-CA | TGt/CAt | C/H | Changed missense |
| SYT8 | 11-1857173-TG-CA | TGc/CAc | C/H | Changed missense |
| SYT8 | 11-1857746-GT-AC | cGT/cAC | R/H | Unchanged |
| OR56A3 | 11-5968728-TG-CA | aTG/aCA | M/T | Partiaiiy changed missense |
| OR56A1 | 11-6048515-TG-CA | cCA/cTG | P/L | Unchanged |
| DNHD1 | 11-6569896-GT-AC | GTg/ACg | V/T | Changed missense |
| DCHS1 | 11-6655433-GT-AC | cAC/cGT | H/R | Unchanged |
| OR2AG2 | 11-6789862-GCT-ACC | AgC/GgT | S/G | Unchanged |
| OVCH2 | 11-7712523-GT-AC | ACa/GTa | T/V | Changed missense |
| TRIM66 | 11-8662090-TG-CA | CAt/TGt | H/C | Changed missense |
| TRIM66 | 11-8662516-TG-CA | CAc/TGc | H/C | Changed missense |
| MRGPRX3 | 11-18159455-TG-CA | TGg/CAg | W/Q | Rescued nonsense |
| MRGPRX3 | 11-18159668-TG-CA | TGg/CAg | W/Q | Rescued nonsense |
| MRGPRX4 | 11-18195173-GTT-ATC | GtT/AtC | V/I | Unchanged |
| RP11-113D6.6 | 11-18210480-TG-CA | cCA/cTG | P/L | Unchanged |
| MRGPRX1 | 11-18956305-GT-AC | gAC/gGT | D/G | Unchanged |
| ALX4 | 11-44297Q54-TG-CA | tCA/tTG | S/L | Unchanged |
| GR10Q1 | 11-57996084-GT-AC | aAC/aGT | N/S | Unchanged |
| RAB3IL1 | 11-61672235-TG-CA | aCA/aTG | T/M | Changed missense |
| ASRGL1 | 11-62105546-GT-CA | GTg/CAg | V/Q | Changed missense |
| CCDC88B | 11-64111928-TG-CA | TGg/CAg | W/Q | Rescued nonsense |
| CCDC88B | 11-64111928-TGG-CGA | TgG/CgA | W/R | Rescued nonsense |
| EFEMP2 | 11-65635801-GTT-ATC | AaC/GaT | N/D | Unchanged |
| C11orf68 | 11-65685225-TG-CA | CAg/TGg | Q/W | Rescued nonsense |
| RHOD | 11-66837965-TG-CA | TGc/CAc | C/H | Changed missense |
| RAD9A | 11-67164789-GGT-AGC | GgT/AgC | G/S | Unchanged |
| MYEOV | 11-69063393-TG-CA | gTG/gCA | V/A | Unchanged |
| KRTAP5-10 | 11-71276715-TG-CA | TGt/CAt | C/H | Changed missense |
| KRTAP5-10 | 11-71276946-GGT-AGC | GgT/AgC | G/S | Unchanged |
| FAM86C1 | 11-71504475-TG-CA | gTG/gCA | V/A | Unchanged |
| ARAP1 | 11-72415329-GT-AC | aAC/aGT | N/S | Unchanged |
| LRRC32 | 11-76372418-TG-CA | aCA/aTG | T/M | Changed missense |
| TMPRSS5 | 11-113570405-TG-CA | gCA/gTG | A/V | Unchanged |
| HYOU1 | 11-118925341-GT-AC | aAC/aGT | N/S | Unchanged |
| MCAM | 11-119181170-TAG-CAA | CtA/TtG | L | Unchanged |
| OR10G7 | 11-123909181-GT-AC | tAC/tGT | Y/C | Unchanged |
| OR10G7 | 11-123909194-TG-CA | CAg/TGg | Q/W | Rescued nonsense |
| HYLS1 | 11-125769354-TG-CA | TGt/CAt | C/H | Changed missense |
| RPUSD4 | 11-126081403-TG-CA | CAg/TGg | Q/W | Rescued nonsense |
| NFRKB | 11-129754668-TG-CA | aCA/aTG | T/M | Changed missense |
| IQSEC3 | 12-284056-GGT-AGC | GgT/AgC | G/S | Unchanged |
| AKAP3 | 12-4737042-GT-AC | cAC/cGT | H/R | Unchanged |
| KCNA5 | 12-5154461-GT-AC | gGT/gAC | G/D | Unchanged |
| VWF | 12-6094784-TG-CA | aCA/aTG | T/M | Changed missense |
| VWF | 12-6128441-GGT-AGC | AcC/GcT | T/A | Unchanged |
| NCAPD2 | 12-6632471-GT-AC | GTg/ACg | V/T | Changed missense |
| KLRC1 | 12-10603669-GT-AC | aAC/aGT | N/S | Unchanged |
| WBP11 | 12-14947554-TG-CA | CAg/TGg | Q/W | Rescued nonsense |
| RERG | 12-15262179-GT-AC | aAC/aGT | N/S | Unchanged |
| ITPR2 | 12-26809316-GT-AC | cAC/cGT | H/R | Unchanged |
| TSPAN11 | 12-31135579-TG-CA | gTG/gCA | V/A | Unchanged |
| LRRK2 | 12-40758652-TG-CA | aTG/aCA | M/T | Partially changed missense |
| MUC19 | 12-40821867-GT-AC | GTc/ACc | V/T | Changed missense |
| OR8S1 | 12-48919793-TG-CA | TGc/CAc | C/H | Changed missense |
| KRT83 | 12-52708419-TG-CA | CAt/TGt | H/C | Changed missense |
| KRT4 | 12-53205757-TG-CA | CAg/TGg | Q/W | Rescued nonsense |
| ESPL1 | 12-53686703-GT-AC | cGT/cAC | R/H | Unchanged |
| SPRYD4 | 12-56862886-TG-CA | aTG/aCA | M/T | Partially changed missense |
| HAL | 12-96384249-TG-CA | cCA/cTG | P/L | Unchanged |
| ANKS1B | 12-99640557-TG-CA | cCA/cTG | P/L | Unchanged |
| GNPTAB | 12-102158763-TG-CA | aCA/aTG | T/M | Changed missense |
| APPL2 | 12-105568176-GT-AC | gAC/gGT | D/G | Unchanged |
| RAB35 | 12-120535169-GT-AC | aAC/aGT | N/S | Unchanged |
| KDM2B | 12-121880520-GT-AC | gAC/gGT | D/G | Unchanged |
| HPD | 12-122295334-GT-AC | ACg/GTg | T/V | Changed missense |
| DDX51 | 12-132626668-TG-CA | CAg/TGg | Q/W | Rescued nonsense |
| GOLGA3 | 12-133389998-TG-CA | aCA/aTG | T/M | Changed missense |
| MIPEP | 13-24411770-GCT-ACC | AgC/GgT | S/G | Unchanged |
| SHISA2 | 13-26620924-GT-AC | aAC/aGT | N/S | Unchanged |
| COG3 | 13-46108853-TG-CA | tTG/tCA | L/S | Unchanged |
| DOCK9 | 13-99452659-GAT-CAA | AtC/TtG | I/L | Changed missense |
| TMTC4 | 13-101287365-TG-CA | gCA/gTG | A/V | Unchanged |
| ARHGEF7 | 13-111870035-GAT-AAC | GaT/AaC | D/N | Unchanged |
| ATP4B | 13-114309226-GT-AC | tAC/tGT | Y/C | Unchanged |
| TMEM255B | 13-114469204-GT-AC | GTt/ACt | V/T | Changed missense |
| OR11H6 | 14-20692643-TG-CA | TGt/CAt | C/H | Changed missense |
| OR4E2 | 14-22133648-GT-AC | GTg/ACg | V/T | Changed missense |
| RABGGTA | 14-24736960-GT-AC | ACg/GTg | T/V | Changed missense |
| SDR39U1 | 14-24909362-TG-CA | CAg/TGg | Q/W | Rescued nonsense |
| CTAGE5 | 14-39736679-GT-AC | GTt/ACt | V/T | Changed missense |
| FBXO34 | 14-55818706-TG-CA | cTG/cCA | L/P | Unchanged |
| ADAM21 | 14-70925520-TG-CA | cTG/cCA | L/P | Unchanged |
| C14orf166B | 14-77332408-TG-CA | gTG/gCA | V/A | Unchanged |
| NGB | 14-77735616-TG-CA | CAg/TGg | Q/W | Rescued nonsense |
| SYNE3 | 14-95912426-TG-CA | gCA/gTG | A/V | Unchanged |
| SYNE3 | 14-95918625-TG-CA | cCA/cTG | P/L | Unchanged |
| AHNAK2 | 14-105413979-TG-CA | aCA/aTG | T/M | Changed missense |
| AHNAK2 | 14-105414132-TG-CA | cCA/cTG | P/L | Unchanged |
| AHNAK2 | 14-105414312-TG-CA | tCA/tTG | S/L | Unchanged |
| AHNAK2 | 14-105415353-GT-AC | gAC/gGT | D/G | Unchanged |
| AHNAK2 | 14-105415482-GT-AC | gAC/gGT | D/G | Unchanged |
| AHNAK2 | 14-105415959-TG-CA | aCA/aTG | T/M | Changed missense |
| AHNAK2 | 14-105416010-TG-CA | aCA/aTG | T/M | Changed missense |
| AHNAK2 | 14-105416619-GTT-ATC | AaC/GaT | N/D | Unchanged |
| AHNAK2 | 14-105416787-TG-CA | tCA/tTG | S/L | Unchanged |
| AHNAK2 | 14-105416790-TG-CA | cCA/cTG | P/L | Unchanged |
| AHNAK2 | 14-105416949-TG-CA | aCA/aTG | T/M | Changed missense |
| AHNAK2 | 14-105417102-TG-CA | cCA/cTG | P/L | Unchanged |
| AHNAK2 | 14-105417444-TG-CA | aCA/aTG | T/M | Changed missense |
| AHNAK2 | 14-105417597-TG-CA | cCA/cTG | P/L | Unchanged |
| AHNAK2 | 14-105417862-TG-CA | CAg/TGg | Q/W | Rescued nonsense |
| AHNAK2 | 14-105418275-TG-CA | cCA/cTG | P/L | Unchanged |
| AHNAK2 | 14-105419424-TG-CA | aCA/aTG | T/M | Changed missense |
| AHNAK2 | 14-105419739-TG-CA | cCA/cTG | P/L | Unchanged |
| IGHE | 14-106066454-TG-CA | gCA/gTG | A/V | Unchanged |
| IGHJ5 | 14-106330035-GT-AC | ACc/GTc | T/V | Changed missense |
| IGHV1-2 | 14-106452734-GCT-ACC | AgC/GgT | S/G | Unchanged |
| IGHV1-24 | 14-106733264-TG-CA | aCA/aTG | T/M | Changed missense |
| IGHV2-26 | 14-106758084-TG-CA | aCA/aTG | T/M | Changed missense |
| IGHV4-39 | 14-106877682-GT-AC | aAC/aGT | N/S | Unchanged |
| IGHV2-70 | 14-107178886-GT-AC | aAC/aGT | N/S | Unchanged |
| GOLGA6L1 | 15-22742549-TG-CA | TGg/CAg | W/Q | Rescued nonsense |
| NIPA1 | 15-23052632-TG-CA | aCA/aTG | T/M | Changed missense |
| GABRA5 | 15-27182355-GTT-ATC | GtT/AtC | V/I | Unchanged |
| HERC2 | 15-28414665-TG-CA | tCA/tTG | S/L | Unchanged |
| GOLGA8M | 15-28949058-GT-AC | cAC/cGT | H/R | Unchanged |
| APBA2 | 15-29347019-TG-CA | cTG/cCA | L/P | Unchanged |
| GOLGA8O | 15-32739268-GT-AC | cAC/cGT | H/R | Unchanged |
| RPAP1 | 15-41821752-TCG-CCA | CgA/TgG | R/W | Rescued nonsense |
| SPTBN5 | 15-42178155-TG-CA | CAc/TGc | H/C | Changed missense |
| IGDCC4 | 15-65676617-TG-CA | cCA/cTG | P/L | Unchanged |
| IGDCC4 | 15-65676617-TGG-CGA | CcA/TcG | P/S | Unchanged |
| IQCH | 15-67692501-GT-AC | cGT/cAC | R/H | Unchanged |
| HCN4 | 15-73614834-TG-CA | cCA/cTG | P/L | Unchanged |
| GOLGA6C | 15-75556026-TG-CA | aTG/aCA | M/T | Partially changed missense |
| GOLGA6D | 15-75582008-TG-CA | TGg/CAg | W/Q | Rescued nonsense |
| CSPG4 | 15-75982185-TG-CA | cCA/cTG | P/L | Unchanged |
| ADAMTS7 | 15-79058695-TG-CA | aCA/aTG | T/M | Changed missense |
| ADAMTS7 | 15-79068536-TG-CA | CAg/TGg | Q/W | Rescued nonsense |
| FAM154B | 15-82574879-TG-CA | TGg/CAg | W/Q | Rescued nonsense |
| FAM154B | 15-82574879-TGG-CGA | TgG/CgA | W/R | Rescued nonsense |
| AKAP13 | 15-86122779-TG-CA | TGg/CAg | W/Q | Rescued nonsense |
| AKAP13 | 15-86124946-TG-CA | aTG/aCA | M/T | Partially changed missense |
| ACAN | 15-89392743-GCT-ACC | GcT/AcC | A/T | Unchanged |
| GDPGP1 | 15-90784250-TG-CA | aTG/aCA | M/T | Partially changed missense |
| SYNM | 15-99653800-TG-CA | gTG/gCA | V/A | Unchanged |
| CERS3 | 15-100943044-GT-AC | gAC/gGT | D/G | Unchanged |
| PIGQ | 16-633353-TG-CA | TGt/CAt | C/H | Changed missense |
| WDR90 | 16-705360-TG-CA | gTG/gCA | V/A | Unchanged |
| WDR90 | 16-705844-TTG-CTA | TtG/CtA | L | Unchanged |
| TPSG1 | 16-1271938-TG-CA | tCA/tTG | S/L | Unchanged |
| ZNF598 | 16-2048704-TG-CA | CAg/TGg | Q/W | Rescued nonsense |
| ZNF598 | 16-2049849-TG-CA | aCA/aTG | T/M | Changed missense |
| TSC2 | 16-2138267-GAT-AAC | GaT/AaC | D/N | Unchanged |
| SLX4 | 16-3650991-TG-CA | cCA/cTG | P/L | Unchanged |
| NAGPA | 16-5075633-GT-AC | ACt/GTt | T/V | Changed missense |
| CTD-3088G3.8 | 16-11540712-TG-CA | aCA/aTG | T/M | Changed missense |
| ERCC4 | 16-14014069-TG-CA | cTG/cCA | L/P | Unchanged |
| SMG1 | 16-18839362-TG-CA | gCA/gTG | A/V | Unchanged |
| ACSM2B | 16-20570659-GAT-AAC | AtC/GtT | I/V | Unchanged |
| DNAH3 | 16-20963765-GGT-CGA | AcC/TcG | T/S | Unchanged |
| SDR42E2 | 16-22178256-GT-AC | cGT/cAC | R/H | Unchanged |
| ZKSCAN2 | 16-25263336-GT-CA | cAC/cTG | H/L | Partially changed missense |
| APOBR | 16-28508716-TG-CA | gTG/gCA | V/A | Unchanged |
| PRRT2 | 16-29825126-TTG-CTA | TtG/CtA | L | Unchanged |
| SEZ6L2 | 16-29883777-TAG-CAA | CtA/TtG | L | Unchanged |
| HSD3B7 | 16-30999461-GT-AC | cGT/cAC | R/H | Unchanged |
| ABCC11 | 16-48201432-TG-CA | CAc/TGc | H/C | Changed missense |
| SLC12A3 | 16-56904140-TG-CA | cTG/cCA | L/P | Unchanged |
| E2F4 | 16-67228632-TG-CA | gTG/gCA | V/A | Unchanged |
| FHOD1 | 16-67266065-TG-CA | aCA/aTG | T/M | Changed missense |
| FAM65A | 16-67563735-TGG-CGA | TgG/CgA | W/R | Rescued nonsense |
| CDH1 | 16-68857439-GCT-ACC | GcT/AcC | A/T | Unchanged |
| COG8 | 16-69373414-TG-CA | aCA/aTG | T/M | Changed missense |
| CLEC18A | 16-69988319-TG-CA | cTG/cCA | L/P | Unchanged |
| CLEC18A | 16-69988372-GT-AC | GTg/ACg | V/T | Changed missense |
| CLEC18A | 16-69988373-TG-CA | gTG/gCA | V/A | Unchanged |
| NPIPB15 | 16-74425793-TCG-CCA | TcG/CcA | S/P | Unchanged |
| TAF1C | 16-84215390-TG-CA | gCA/gTG | A/V | Unchanged |
| ZCCHC14 | 16-87445363-GT-AC | cAC/cGT | H/R | Unchanged |
| PIEZO1 | 16-88783098-GAT-AAC | AtC/GtT | I/V | Unchanged |
| PIEZO1 | 16-88791457-TG-CA | cCA/cTG | P/L | Unchanged |
| PIEZO1 | 16-88807895-TG-CA | cCA/cTG | P/L | Unchanged |
| ACSF3 | 16-89167402-GAT-AAC | GaT/AaC | D/N | Unchanged |
| MYO1C | 17-1373518-TG-CA | CAg/TGg | Q/W | Rescued nonsense |
| WDR81 | 17-1630991-TG-CA | cTG/cCA | L/P | Unchanged |
| SMYD4 | 17-1686409-GT-AC | tAC/tGT | Y/C | Unchanged |
| MNT | 17-2298615-TG-CA | cCA/cTG | P/L | Unchanged |
| ITGAE | 17-3657173-GAT-AAC | AtC/GtT | I/V | Unchanged |
| ZZEF1 | 17-3912208-GT-AC | ACg/GTg | T/V | Changed missense |
| ZZEF1 | 17-3953083-TG-CA | cCA/cTG | P/L | Unchanged |
| GLTPD2 | 17-4693170-TG-CA | gTG/gCA | V/A | Unchanged |
| AIPL1 | 17-6330068-TG-CA | cCA/cTG | P/L | Unchanged |
| SLC35G6 | 17-7386279-TG-CA | TGg/CAg | W/Q | Rescued nonsense |
| PER1 | 17-8048169-TG-CA | aCA/aTG | T/M | Changed missense |
| PFAS | 17-8167600-TG-CA | cTG/cCA | L/P | Unchanged |
| MYH3 | 17-10543463-TG-CA | gCA/gTG | A/V | Unchanged |
| CDRT1 | 17-15510986-GTT-ATC | AaC/GaT | N/D | Unchanged |
| RP11-385D13.1 | 17-15510986-GTT-ATC | AaC/GaT | N/D | Unchanged |
| NOS2 | 17-26115949-TG-CA | cCA/cTG | P/L | Unchanged |
| ABHD15 | 17-27889985-GT-AC | ACc/GTc | T/V | Changed missense |
| SLFN14 | 17-33884804-TG-CA | CAg/TGg | Q/W | Rescued nonsense |
| KRT28 | 17-38950272-TG-CA | aCA/aTG | T/M | Changed missense |
| KRTAP1-4 | 17-39186097-GT-AC | tAC/tGT | Y/C | Unchanged |
| KRT32 | 17-39619093-GT-AC | aAC/aGT | N/S | Unchanged |
| KRT32 | 17-39623363-TG-CA | CAg/TGg | Q/W | Rescued nonsense |
| RUNDC1 | 17-41133071-TG-CA | TGg/CAg | W/Q | Rescued nonsense |
| MPP2 | 17-41960343-TG-CA | aCA/aTG | T/M | Changed missense |
| MPP2 | 17-41960634-GT-CA | cAC/cTG | H/L | Partially changed missense |
| KIF18B | 17-43003484-TG-CA | gCA/gTG | A/V | Unchanged |
| WNT9B | 17-44950122-TG-CA | aTG/aCA | M/T | Partially changed missense |
| B4GALNT2 | 17-47246163-TG-CA | TGc/CAc | C/H | Changed missense |
| COL1A1 | 17-48265493-GGT-AGC | AcC/GcT | T/A | Unchanged |
| RSAD1 | 17-48557348-TG-CA | tTG/tCA | L/S | Unchanged |
| BZRAP1 | 17-56396609-TG-CA | CAg/TGg | Q/W | Rescued nonsense |
| MTMR4 | 17-56572524-TG-CA | aCA/aTG | T/M | Changed missense |
| MTMR4 | 17-56584506-GCT-ACC | AgC/GgT | S/G | Unchanged |
| C17orf47 | 17-56621359-TG-CA | tCA/tTG | S/L | Unchanged |
| KCNH6 | 17-61611421-GAT-AAC | GaT/AaC | D/N | Unchanged |
| ARSG | 17-66364804-TG-CA | TGg/CAg | W/Q | Rescued nonsense |
| C17orf80 | 17-71232807-TG-CA | TGt/CAt | C/H | Changed missense |
| TTYH2 | 17-72233559-GT-AC | GTt/ACt | V/T | Changed missense |
| NAT9 | 17-72769136-TG-CA | CAg/TGg | Q/W | Rescued nonsense |
| RECQL5 | 17-73625430-GT-AC | cAC/cGT | H/R | Unchanged |
| RECQL5 | 17-73625478-TG-CA | aCA/aTG | T/M | Changed missense |
| EVPL | 17-74014668-TG-CA | CAg/TGg | Q/W | Rescued nonsense |
| QRICH2 | 17-74288262-TG-CA | CAt/TGt | H/C | Changed missense |
| C17orf99 | 17-76157266-TG-CA | TGg/CAg | W/Q | Rescued nonsense |
| TK1 | 17-76178748-TG-CA | gCA/gTG | A/V | Unchanged |
| C1QTNF1 | 17-77040127-GT-AC | cGT/cAC | R/H | Unchanged |
| ENPP7 | 17-77704912-TG-CA | cTG/cCA | L/P | Unchanged |
| ENPP7 | 17-77705043-GAT-AAC | GaT/AaC | D/N | Unchanged |
| SLC38A10 | 17-79220016-TG-CA | gCA/gTG | A/V | Unchanged |
| TSPAN10 | 17-79612382-TG-CA | gTG/gCA | V/A | Unchanged |
| SLC25A10 | 17-79682557-GT-AC | cGT/cAC | R/H | Unchanged |
| PYCR1 | 17-79891147-TG-CA | tCA/tTG | S/L | Unchanged |
| ASPSCR1 | 17-79974729-GAT-AAC | GaT/AaC | D/N | Unchanged |
| ZNF750 | 17-80788465-TG-CA | gCA/gTG | A/V | Unchanged |
| TBCD | 17-80887242-GAT-AAC | GaT/AaC | D/N | Unchanged |
| COLEC12 | 18-334758-TG-CA | cCA/cTG | P/L | Unchanged |
| LAMA1 | 18-6999628-TG-CA | tCA/tTG | S/L | Unchanged |
| MOCOS | 18-33848581-TG-CA | gTG/gCA | V/A | Unchanged |
| MRO | 18-48331524-GT-AC | gAC/gGT | D/G | Unchanged |
| SERPINB4 | 18-61308176-GT-AC | ACt/GTt | T/V | Changed missense |
| ZNF516 | 18-74154999-GT-AC | aAC/aGT | N/S | Unchanged |
| LPPR3 | 19-813219-GT-AC | ACg/GTg | T/V | Changed missense |
| GRIN3B | 19-1004740-TG-CA | TGg/CAg | W/Q | Rescued nonsense |
| MOB3A | 19-2078488-TG-CA | cCA/cTG | P/L | Unchanged |
| CACTIN | 19-3611972-GT-AC | aAC/aGT | N/S | Unchanged |
| TJP3 | 19-3750615-TG-CA | aTG/aCA | M/T | Partially changed missense |
| FSD1 | 19-4306310-GT-AC | cGT/cAC | R/H | Unchanged |
| TICAM1 | 19-4818378-TG-CA | aCA/aTG | T/M | Changed missense |
| FUT5 | 19-5866736-TG-CA | CAc/TGc | H/C | Changed missense |
| EMR1 | 19-6926378-TG-CA | aTG/aCA | M/T | Partially changed missense |
| AC025278.1 | 19-7011826-TG-CA | TGg/CAg | W/Q | Rescued nonsense |
| STXBP2 | 19-7711219-GAT-AAC | GaT/AaC | D/N | Unchanged |
| LRRC8E | 19-7963976-TG-CA | aTG/aCA | M/T | Partially changed missense |
| FBN3 | 19-8181644-TG-CA | CAa/TGa | Q/* | Unchanged |
| MBD3L1 | 19-8953799-GTT-ATC | GtT/AtC | V/I | Unchanged |
| MUC16 | 19-9003645-GT-AC | ACt/GTt | T/V | Changed missense |
| MUC16 | 19-9048421-TG-CA | aCA/aTG | T/M | Changed missense |
| MUC16 | 19-9060812-TG-CA | tCA/tTG | S/L | Unchanged |
| DNMT1 | 19-10291181-TG-CA | CAt/TGt | H/C | Changed missense |
| ICAM3 | 19-10449512-GAT-AAC | AtC/GtT | I/V | Unchanged |
| DOCK6 | 19-11319491-GT-AC | aAC/aGT | N/S | Unchanged |
| DOCK6 | 19-11319491-GTT-ATC | AaC/GaT | N/D | Unchanged |
| PRKCSH | 19-11553279-GCT-ACC | GcT/AcC | A/T | Unchanged |
| RNASEH2A | 19-12921186-TG-CA | tTG/tCA | L/S | Unchanged |
| DNASE2 | 19-12989560-GCT-ACC | AgC/GgT | S/G | Unchanged |
| C19orf57 | 19-14000170-TG-CA | CAg/TGg | Q/W | Rescued nonsense |
| OR7A5 | 19-14938833-GT-AC | ACt/GTt | T/V | Changed missense |
| NOTCH3 | 19-15302844-TG-CA | gCA/gTG | A/V | Unchanged |
| CYP4F3 | 19-15763720-TG-CA | gTG/gCA | V/A | Unchanged |
| OR10H5 | 19-15905271-TG-CA | cTG/cCA | L/P | Unchanged |
| OR10H1 | 19-15918500-GGT-AGC | AcC/GcT | T/A | Unchanged |
| CPAMD8 | 19-17004049-TG-CA | CAg/TGg | Q/W | Rescued nonsense |
| MYO9B | 19-17316781-GT-AC | GTt/ACt | V/T | Changed missense |
| IL12RB1 | 19-18186618-TG-CA | CAg/TGg | Q/W | Rescued nonsense |
| KIAA1683 | 19-18375815-GT-AC | aAC/aGT | N/S | Unchanged |
| COPE | 19-19011244-TG-CA | cCA/cTG | P/L | Unchanged |
| ZNF729 | 19-22498942-GT-AC | tGT/tAC | C/Y | Unchanged |
| APLP1 | 19-36370010-TG-CA | gTG/gCA | V/A | Unchanged |
| LGALS14 | 19-40197924-TG-CA | TGt/CAt | C/H | Changed missense |
| PSG8 | 19-43269704-TG-CA | aCA/aTG | T/M | Changed missense |
| PSG4 | 19-43709659-TG-CA | aCA/aTG | T/M | Changed missense |
| ZNF221 | 19-44470420-TG-CA | TGt/CAt | C/H | Changed missense |
| ZNF180 | 19-44981832-TG-CA | CAt/TGt | H/C | Changed missense |
| PPP5D1 | 19-47030234-TG-CA | gCA/gTG | A/V | Unchanged |
| CCDC9 | 19-47774772-TG-CA | cTG/cCA | L/P | Unchanged |
| VRK3 | 19-50491733-GCT-ACC | AgC/GgT | S/G | Unchanged |
| KLK14 | 19-51584951-TG-CA | CAg/TGg | Q/W | Rescued nonsense |
| ZNF577 | 19-52376119-GT-AC | ACt/GTt | T/V | Changed missense |
| ZNF615 | 19-52497745-TG-CA | CAg/TGg | Q/W | Rescued nonsense |
| VN1R2 | 19-53761740-TG-CA | TGc/CAc | C/H | Changed missense |
| MBOAT7 | 19-54677759-TG-CA | cCA/cTG | P/L | Unchanged |
| MBOAT7 | 19-54677759-TGG-CGA | CcA/TcG | P/S | Unchanged |
| LILRB3 | 19-54726226-TG-CA | gCA/gTG | A/V | Unchanged |
| LILRA4 | 19-54844900-TG-CA | gCA/gTG | A/V | Unchanged |
| KIR2DL1 | 19-55286650-TG-CA | cTG/cCA | L/P | Unchanged |
| TMEM86B | 19-55738634-TG-CA | CAc/TGc | H/C | Changed missense |
| SSC5D | 19-56024519-TG-CA | cTG/cCA | L/P | Unchanged |
| ZSCAN5D | 19-56751782-TG-CA | TGc/CAc | C/H | Changed missense |
| ZNF749 | 19-57956186-TG-CA | gTG/gCA | V/A | Unchanged |
| ADCY3 | 2-25046090-TG-CA | tCA/tTG | S/L | Unchanged |
| SLC5A6 | 2-27423929-TG-CA | cCA/cTG | P/L | Unchanged |
| AC074091.13 | 2-27930502-GT-CA | aAC/aTG | N/M | Changed missense |
| FAM179A | 2-29246044-TG-CA | gTG/gCA | V/A | Unchanged |
| ARHGAP25 | 2-69049941-TG-CA | aTG/aCA | M/T | Partially changed missense |
| ASPRV1 | 2-70188675-GT-AC | ACt/GTt | T/V | Changed missense |
| REG3G | 2-79253947-TG-CA | aTG/aCA | M/T | Partially changed missense |
| CTNNA2 | 2-80801344-GTT-ATC | GtT/AtC | V/I | Unchanged |
| IGKJ3 | 2-89160737-GAT-AAC | AtC/GtT | I/V | Unchanged |
| IGKV1-5 | 2-89247011-GCT-ACC | AgC/GgT | S/G | Unchanged |
| IGKV1-8 | 2-89292169-GT-AC | ACa/GTa | T/V | Changed missense |
| IGKV3-15 | 2-89384784-GAT-AAC | AtC/GtT | I/V | Unchanged |
| IGKV3-15 | 2-89384814-GAT-CAA | AtC/TtG | I/L | Changed missense |
| TUBA3E | 2-130951584-TG-CA | tCA/tTG | S/L | Unchanged |
| LRP2 | 2-170103336-TG-CA | aCA/aTG | T/M | Changed missense |
| TTN | 2-179430997-GT-AC | ACg/GTg | T/V | Changed missense |
| FSIP2 | 2-186656956-TG-CA | aTG/aCA | M/T | Partially changed missense |
| SLC40A1 | 2-190430141-TAG-CAA | CtA/TtG | L | Unchanged |
| MYO1B | 2-192279327-TTG-CTA | TtG/CtA | L | Unchanged |
| RAPH1 | 2-204305093-TG-CA | cCA/cTG | P/L | Unchanged |
| PIKFYVE | 2-209190330-TG-CA | tTG/tCA | L/S | Unchanged |
| ABCA12 | 2-215865575-TG-CA | cCA/cTG | P/L | Unchanged |
| IRS1 | 2-227661043-TG-CA | gCA/gTG | A/V | Unchanged |
| COL4A3 | 2-228111435-TG-CA | cTG/cCA | L/P | Unchanged |
| SP140 | 2-231175549-TG-CA | TGt/CAt | C/H | Changed missense |
| SP100 | 2-231406680-TG-CA | aTG/aCA | M/T | Partially changed missense |
| UGT1A5 | 2-234621780-TG-CA | tTG/tCA | L/S | Unchanged |
| UGT1A5 | 2-234621810-TG-CA | gTG/gCA | V/A | Unchanged |
| ESPNL | 2-239039637-TG-CA | gTG/gCA | V/A | Unchanged |
| ESPNL | 2-239040185-GGT-AGC | GgT/AgC | G/S | Unchanged |
| OR6B3 | 2-240985347-GT-AC | ACc/GTc | T/V | Changed missense |
| PASK | 2-242066374-GT-AC | aAC/aGT | N/S | Unchanged |
| SIRPG | 20-1610894-GT-AC | tAC/tGT | Y/C | Unchanged |
| TGM6 | 20-2375106-GT-AC | GTc/ACc | V/T | Changed missense |
| BFSP1 | 20-17474968-TG-CA | cCA/cTG | P/L | Unchanged |
| CSTL1 | 20-23424613-TG-CA | TGg/CAg | W/Q | Rescued nonsense |
| CST3 | 20-23618395-TAG-CAA | CtA/TtG | L | Unchanged |
| PYGB | 20-25264812-GAT-AAC | GaT/AaC | D/N | Unchanged |
| SEMG2 | 20-43851094-GT-AC | aGT/aAC | S/N | Unchanged |
| PREX1 | 20-47249170-GT-AC | aAC/aGT | N/S | Unchanged |
| PCK1 | 20-56139402-GT-AC | gGT/gAC | G/D | Unchanged |
| ZBP1 | 20-56186884-TG-CA | CAg/TGg | Q/W | Rescued nonsense |
| GATA5 | 20-61039958-TG-CA | cCA/cTG | P/L | Unchanged |
| C20orf166 | 20-61162266-GT-AC | GTg/ACg | V/T | Changed missense |
| C20orf166 | 20-61162267-TG-CA | gTG/gCA | V/A | Unchanged |
| C20orf166 | 20-61167882-TG-CA | TGa/CAa | */Q | Unchanged |
| DIDO1 | 20-61528074-TG-CA | gCA/gTG | A/V | Unchanged |
| TNFRSF6B | 20-62328452-GT-AC | cGT/cAC | R/H | Unchanged |
| MX1 | 21-42821112-GT-AC | cGT/cAC | R/H | Unchanged |
| TMPRSS2 | 21-42866296-TG-CA | aCA/aTG | T/M | Changed missense |
| UMODL1 | 21-43531008-TG-CA | aTG/aCA | M/T | Partially changed missense |
| RRP1 | 21-45219522-GTT-ATC | GtT/AtC | V/I | Unchanged |
| C21orf33 | 21-45553595-GT-AC | GTc/ACc | V/T | Changed missense |
| AIRE | 21-45717548-GAT-AAC | GaT/AaC | D/N | Unchanged |
| KRTAP10-1 | 21-45959854-TG-CA | gCA/gTG | A/V | Unchanged |
| KRTAP10-2 | 21-45971225-TG-CA | cCA/cTG | P/L | Unchanged |
| KRTAP10-3 | 21-45977994-GT-AC | ACg/GTg | T/V | Changed missense |
| KRTAP10-3 | 21-45978338-TG-CA | cCA/cTG | P/L | Unchanged |
| KRTAP10-6 | 21-46011718-TG-CA | cCA/cTG | P/L | Unchanged |
| KRTAP10-6 | 21-46011718-TGG-CGA | CcA/TcG | P/S | Unchanged |
| KRTAP10-6 | 21-46012154-GT-AC | ACc/GTc | T/V | Changed missense |
| COL6A1 | 21-47410929-GGT-AGC | GgT/AgC | G/S | Unchanged |
| COL6A2 | 21-47538960-GT-AC | aGT/aAC | S/N | Unchanged |
| PCNT | 21-47787001-GT-AC | GTg/ACg | V/T | Changed missense |
| PCNT | 21-47787002-TG-CA | gTG/gCA | V/A | Unchanged |
| AC008132.13 | 22-18835523-TG-CA | gTG/gCA | V/A | Unchanged |
| IGLV7-46 | 22-22724394-TG-CA | tTG/tCA | L/S | Unchanged |
| IGLV1-40 | 22-22764409-GGT-CGA | GgT/CgA | G/R | Unchanged |
| IGLV2-23 | 22-23040770-GT-CA | aGT/aCA | S/T | Partially changed missense |
| IGLV2-11 | 22-23135287-GT-CA | gGT/gCA | G/A | Unchanged |
| IGLV3-1 | 22-23223435-GAT-AAC | GaT/AaC | D/N | Unchanged |
| CABIN1 | 22-24459437-GT-AC | tGT/tAC | C/Y | Unchanged |
| MYO18B | 22-26194044-GT-AC | gGT/gAC | G/D | Unchanged |
| SEC14L6 | 22-30934816-TG-CA | CAa/TGa | Q/* | Unchanged |
| MORC2 | 22-31333631-TG-CA | cCA/cTG | P/L | Unchanged |
| APOL1 | 22-36661565-TG-CA | aTG/aCA | M/T | Partially changed missense |
| C22orf23 | 22-38341134-TG-CA | aCA/aTG | T/M | Changed missense |
| APOBEC3F | 22-39448283-TG-CA | TGg/CAg | W/Q | Rescued nonsense |
| ARFGAP3 | 22-43195109-TG-CA | CAg/TGg | Q/W | Rescued nonsense |
| PARVB | 22-44395451-TG-CA | TGg/CAg | W/Q | Rescued nonsense |
| PRR5 | 22-45075738-TG-CA | aTG/aCA | M/T | Partially changed missense |
| C22orf26 | 22-46447714-TG-CA | CAg/TGg | Q/W | Rescued nonsense |
| CTA-299D3.8 | 22-48940551-GCT-ACC | AgC/GgT | S/G | Unchanged |
| DENND6B | 22-50754648-TG-CA | gCA/gTG | A/V | Unchanged |
| LMF2 | 22-50944128-GT-AC | cAC/cGT | H/R | Unchanged |
| EFHB | 3-19959756-GT-AC | ACc/GTc | T/V | Changed missense |
| TRANK1 | 3-36898715-GT-AC | ACc/GTc | T/V | Changed missense |
| PLCD1 | 3-38051941-TG-CA | cCA/cTG | P/L | Unchanged |
| XYLB | 3-38401838-GT-AC | gGT/gAC | G/D | Unchanged |
| SCN5A | 3-38674712-TG-CA | gCA/gTG | A/V | Unchanged |
| XIRP1 | 3-39229900-TG-CA | CAg/TGg | Q/W | Rescued nonsense |
| CDCP1 | 3-45134822-TG-CA | CAa/TGa | Q/* | Unchanged |
| CCDC71 | 3-49200692-TG-AC | CAg/GTg | Q/V | Changed missense |
| BSN | 3-49693411 -TG-CA | cTG/cCA | L/P | Unchanged |
| GMPPB | 3-49760039-TG-CA | CAg/TGg | Q/W | Rescued nonsense |
| CAMKV | 3-49897244-TG-AC | CAg/GTg | Q/V | Changed missense |
| PCBP4 | 3-51994587-TG-CA | gCA/gTG | A/V | Unchanged |
| RP11- | 3-51994587-TG-CA | gCA/gTG | A/V | Unchanged |
| 155D18.14 | | | | |
| PRKCD | 3-53213655-GAT-AAC | GaT/AaC | D/N | Unchanged |
| FLNB | 3-58118555-GT-AC | GTg/ACg | V/T | Changed missense |
| FRMD4B | 3-69245453-GT-AC | ACg/GTg | T/V | Changed missense |
| EPHA3 | 3-89521693-TG-CA | TGg/CAg | W/Q | Rescued nonsense |
| OR5K1 | 3-98189157-TG-CA | tTG/tCA | L/S | Unchanged |
| DPPA4 | 3-109049384-TG-CA | cCA/cTG | P/L | Unchanged |
| FBXO40 | 3-121340536-TG-CA | gTG/gCA | V/A | Unchanged |
| GOLGB1 | 3-121435621-TTG-CTA | CaA/TaG | Q/* | Unchanged |
| MYLK | 3-123452838-GGT-AGC | AcC/GcT | T/A | Unchanged |
| ALDH1L1 | 3-125826058-GT-AC | gAC/gGT | D/G | Unchanged |
| EFCAB12 | 3-129140489-TG-CA | aCA/aTG | T/M | Changed missense |
| PLXND1 | 3-129293182-GT-AC | cAC/cGT | H/R | Unchanged |
| PLXND1 | 3-129293183-TG-CA | CAc/TGc | H/C | Changed missense |
| SLCO2A1 | 3-133670073-TCG-CCA | CgA/TgG | R/W | Rescued nonsense |
| DZIP1L | 3-137796377-GGT-AGC | AcC/GcT | T/A | Unchanged |
| HLTF | 3-148759325-TG-CA | gCA/gTG | A/V | Unchanged |
| IGSF10 | 3-151154666-TG-CA | aCA/aTG | T/M | Changed missense |
| RARRES1 | 3-158450079-GT-AC | gAC/gGT | D/G | Unchanged |
| NCEH1 | 3-172428782-TG-CA | CAg/TGg | Q/W | Rescued nonsense |
| AHSG | 3-186337713-TG-CA | aTG/aCA | M/T | Partially changed missense |
| LRRC15 | 3-194081152-GTT-ATC | AaC/GaT | N/D | Unchanged |
| LRRC15 | 3-194081635-TG-CA | gCA/gTG | A/V | Unchanged |
| MUC4 | 3-195505662-TGG-CGA | CcA/TcG | P/S | Unchanged |
| MUC4 | 3-195507322-GT-AC | ACc/GTc | T/V | Changed missense |
| MUC4 | 3-195515386-GT-AC | ACc/GTc | T/V | Changed missense |
| MUC4 | 3-195515482-GT-AC | ACc/GTc | T/V | Changed missense |
| PIGZ | 3-196674973-TG-CA | gCA/gTG | A/V | Unchanged |
| MFI2 | 3-196742290-TG-CA | cCA/cTG | P/L | Unchanged |
| GAK | 4-858941-TG-CA | gCA/gTG | A/V | Unchanged |
| SLC26A1 | 4-983060-TG-CA | CAg/TGg | Q/W | Rescued nonsense |
| FAM53A | 4-1657281-GGT-AGC | AcC/GcT | T/A | Unchanged |
| HAUS3 | 4-2242083-TTG-ATC | CaA/GaT | Q/D | Changed missense |
| GRK4 | 4-3037213-TG-CA | cTG/cCA | L/P | Unchanged |
| GRK4 | 4-3039150-TG-CA | gTG/gCA | V/A | Unchanged |
| LYAR | 4-4276131-GT-AC | cAC/cGT | H/R | Unchanged |
| LYAR | 4-4276132-TG-CA | CAc/TGc | H/C | Changed missense |
| EVC2 | 4-5624669-GT-AC | ACg/GTg | T/V | Changed missense |
| PSAPL1 | 4-7436151-TG-CA | cCA/cTG | P/L | Unchanged |
| SORCS2 | 4-7714488-GAT-AAC | GaT/AaC | D/N | Unchanged |
| USP17L10 | 4-9213568-GT-AC | GTa/ACa | V/T | Changed missense |
| PPARGC1A | 4-23815924-TG-CA | aCA/aTG | T/M | Changed missense |
| RFC1 | 4-39302029-TG-CA | cCA/cTG | P/L | Unchanged |
| NFXL1 | 4-47887536-TG-CA | gCA/gTG | A/V | Unchanged |
| FRYL | 4-48582909-GCT-ACC | AgC/GgT | S/G | Unchanged |
| CLOCK | 4-56314977-GT-AC | ACa/GTa | T/V | Changed missense |
| NOA1 | 4-57829649-GT-AC | aAC/aGT | N/S | Unchanged |
| TMPRSS11A | 4-68784774-TG-CA | CAg/TGg | Q/W | Rescued nonsense |
| UGT2B17 | 4-69416556-TG-CA | gCA/gTG | A/V | Unchanged |
| UGT2B4 | 4-70346496-GT-AC | cAC/cGT | H/R | Unchanged |
| SMR3A | 4-71232430-TG-CA | TGt/CAt | C/H | Changed missense |
| HSD17B11 | 4-88258483-TG-CA | CAa/TGa | Q/* | Unchanged |
| HERC6 | 4-89311962-TG-CA | TGt/CAt | C/H | Changed missense |
| SMARCAD1 | 4-95170839-GT-AC | aGT/aAC | S/N | Unchanged |
| ALPK1 | 4-113353285-TG-CA | aTG/aCA | M/T | Partially changed missense |
| HSPA4L | 4-128723042-TG-CA | tTG/tCA | L/S | Unchanged |
| PCDH18 | 4-138442525-GGT-AGC | AcC/GcT | T/A | Unchanged |
| KIAA0922 | 4-154479429-GT-AC | cGT/cAC | R/H | Unchanged |
| PALLD | 4-169433326-TG-CA | aTG/aCA | M/T | Partially changed missense |
| CCDC110 | 4-186379371-GT-AC | tAC/tGT | Y/C | Unchanged |
| CYP4V2 | 4-187122354-GT-AC | tGT/tAC | C/Y | Unchanged |
| FAT1 | 4-187557893-TG-CA | CAc/TGc | H/C | Changed missense |
| SDHA | 5-228312-GAT-AAC | GaT/AaC | D/N | Unchanged |
| SLC12A7 | 5-1063998-GT-AC | tAC/tGT | Y/C | Unchanged |
| CCT5 | 5-10256160-GT-AC | cGT/cAC | R/H | Unchanged |
| SLC45A2 | 5-33954511-TG-CA | aCA/aTG | T/M | Changed missense |
| EGFLAM | 5-38370537-TG-CA | TGg/CAg | W/Q | Rescued nonsense |
| MROH2B | 5-41000343-GT-AC | tAC/tGT | Y/C | Unchanged |
| MROH2B | 5-41057374-TG-CA | CAg/TGg | Q/W | Rescued nonsense |
| BDP1 | 5-708Q6958-GT-AC | GTg/ACg | V/T | Changed missense |
| TNPO1 | 5-72183986-TG-CA | gTG/gCA | V/A | Unchanged |
| GDF9 | 5-132198199-GGT-AGC | AcC/GcT | T/A | Unchanged |
| SEC24A | 5-134032907-GT-CA | aGT/aCA | S/T | Partially changed missense |
| TMEM173 | 5-138857919-TG-CA | CAt/TGt | H/C | Changed missense |
| WDR55 | 5-140048267-TG-CA | TGt/CAt | C/H | Changed missense |
| PCDHA1 | 5-140166017-GTT-ATC | GtT/AtC | V/I | Unchanged |
| PCDHA11 | 5-140250252-TTG-CTA | TtG/CtA | L | Unchanged |
| PCDHA13 | 5-140263420-TTG-CTA | TtG/CtA | L | Unchanged |
| PCDHB5 | 5-14Q517174-TCG-CCA | TcG/CcA | S/P | Unchanged |
| PCDHB16 | 5-140563980-GGT-AGC | GgT/AgC | G/S | Unchanged |
| SPINKS | 5-147510865-GT-AC | gGT/gAC | G/D | Unchanged |
| SH3TC2 | 5-148386525-TG-CA | cCA/cTG | P/L | Unchanged |
| TIGD6 | 5-149374932-TG-CA | CAg/TGg | Q/W | Rescued nonsense |
| CSF1R | 5-149450132-TG-CA | CAc/TGc | H/C | Changed missense |
| PDGFRB | 5-149495395-TG-CA | cCA/cTG | P/L | Unchanged |
| THG1L | 5-157164962-TG-CA | cTG/cCA | L/P | Unchanged |
| CCNJL | 5-159686502-TG-CA | CAt/TGt | H/C | Changed missense |
| STK10 | 5-171520554-TG-CA | cCA/cTG | P/L | Unchanged |
| AC027309.1 | 5-172036332-TG-CA | aCA/aTG | T/M | Changed missense |
| CDHR2 | 5-176004476-TG-CA | gTG/gCA | V/A | Unchanged |
| ZNF354B | 5-178293284-TGG-CGA | TgG/CgA | W/R | Rescued nonsense |
| SLC22A23 | 6-3287118-TG-CA | tCA/tTG | S/L | Unchanged |
| MCUR1 | 6-13814338-GCT-ACC | AgC/GgT | S/G | Unchanged |
| GPLD1 | 6-24437445-GT-AC | ACa/GTa | T/V | Changed missense |
| BTN2A1 | 6-26468434-TG-CA | cTG/cCA | L/P | Unchanged |
| HLA-C | 6-31237771-GGT-AGC | AcC/GcT | T/A | Unchanged |
| MICA | 6-31379118-GT-AC | GTa/ACa | V/T | Changed missense |
| ABHD16A | 6-31657413-TAG-CAA | CtA/TtG | L | Unchanged |
| NOTCH4 | 6-32188642-TG-CA | cCA/cTG | P/L | Unchanged |
| HLA-DRB5 | 6-32489939-TG-CA | CAg/TGg | Q/W | Rescued nonsense |
| TAP1 | 6-32814974-GT-AC | gAC/gGT | D/G | Unchanged |
| HLA-DPA1 | 6-33037419-GT-AC | aAC/aGT | N/S | Unchanged |
| RPS10 | 6-34392586-TG-AC | CAg/GTg | Q/V | Changed missense |
| RPS10-NUDT3 | 6-34392586-TG-AC | CAg/GTg | Q/V | Changed missense |
| TFEB | 6-41652604-TG-CA | cCA/cTG | P/L | Unchanged |
| TFEB | 6-41652604-TGG-CGA | CcA/TcG | P/S | Unchanged |
| CUL7 | 6-43014298-TTG-CTA | CaA/TaG | Q/* | Unchanged |
| CUL7 | 6-43014299-TG-CA | CAa/TGa | Q/* | Unchanged |
| DLK2 | 6-43418733-GT-AC | cAC/cGT | H/R | Unchanged |
| SLC25A27 | 6-46623696-GCT-ACC | GcT/AcC | A/T | Unchanged |
| GPR116 | 6-46826844-TG-CA | aCA/aTG | T/M | Changed missense |
| ICK | 6-52878637-TG-CA | cCA/cTG | P/L | Unchanged |
| MDN1 | 6-90482395-GAT-AAC | AtC/GtT | I/V | Unchanged |
| FAXC | 6-99771540-TG-CA | aCA/aTG | T/M | Changed missense |
| PTPRK | 6-128404875-GAT-AAC | AtC/GtT | I/V | Unchanged |
| CTAGE9 | 6-132030028-TG-CA | aCA/aTG | T/M | Changed missense |
| ESR1 | 6-152201874-GT-AC | cGT/cAC | R/H | Unchanged |
| RPS6KA2 | 6-167271710-GT-AC | ACc/GTc | T/V | Changed missense |
| RP11-514012.4 | 6-167271710-GT-AC | ACc/GTc | T/V | Changed missense |
| WDR27 | 6-169972595-GT-AC | cAC/cGT | H/R | Unchanged |
| WDR27 | 6-169972596-TG-CA | CAc/TGc | H/C | Changed missense |
| HEATR2 | 7-810218-GT-AC | GTg/ACg | V/T | Changed missense |
| TMEM184A | 7-1586660-GCT-ACC | AgC/GgT | S/G | Unchanged |
| CARD11 | 7-2946461-TCG-CCA | CgA/TgG | R/W | Rescued nonsense |
| COL28A1 | 7-7413149-TG-CA | cCA/cTG | P/L | Unchanged |
| THSD7A | 7-11521542-TG-CA | cCA/cTG | P/L | Unchanged |
| THSD7A | 7-11581134-TG-CA | gCA/gTG | A/V | Unchanged |
| AGMO | 7-15458186-TG-CA | aCA/aTG | T/M | Changed missense |
| STEAP1B | 7-22533235-TG-CA | CAt/TGt | H/C | Changed missense |
| C7orf31 | 7-25181912-TG-CA | CAt/TGt | H/C | Changed missense |
| SCRN1 | 7-29966141-TG-CA | CAg/TGg | Q/W | Rescued nonsense |
| SPDYE1 | 7-44047066-TG-CA | TGc/CAc | C/H | Changed missense |
| NACAD | 7-45120248-GT-AC | aAC/aGT | N/S | Unchanged |
| C7orf57 | 7-48092484-GAT-AAC | GaT/AaC | D/N | Unchanged |
| ZNF680 | 7-64004108-TG-CA | CAg/TGg | Q/W | Rescued nonsense |
| POM121C | 7-75051375-TG-CA | cCA/cTG | P/L | Unchanged |
| SRCRB4D | 7-76028057-GT-AC | ACg/GTg | T/V | Changed missense |
| SEMA3A | 7-83590852-TG-CA | aCA/aTG | T/M | Changed missense |
| MCM7 | 7-99693078-GAT-AAC | AtC/GtT | l/V | Unchanged |
| ZAN | 7-100386900-TG-CA | TGg/CAg | W/Q | Rescued nonsense |
| MUC17 | 7-100676793-TG-CA | cTG/cCA | L/P | Unchanged |
| LAMB4 | 7-107738882-GT-AC | tAC/tGT | Y/C | Unchanged |
| FLNC | 7-128487864-GAT-AAC | GaT/AaC | D/N | Unchanged |
| PLXNA4 | 7-131864591-TG-CA | gCA/gTG | A/V | Unchanged |
| CNOT4 | 7-135047703-TG-CA | aCA/aTG | T/M | Changed missense |
| DENND2A | 7-140258000-TG-CA | tCA/tTG | S/L | Unchanged |
| TAS2R38 | 7-141672602-GAT-AAC | AtC/GtT | I/V | Unchanged |
| TRBV5-6 | 7-142131851-TG-CA | gCA/gTG | A/V | Unchanged |
| TRPV5 | 7-142625249-TG-CA | aCA/aTG | T/M | Changed missense |
| TAS2R60 | 7-143141474-GT-AC | cGT/cAC | R/H | Unchanged |
| ACTR3C | 7-149983565-TG-CA | CAg/TGg | Q/W | Rescued nonsense |
| WRN | 8-31024654-TG-CA | TGt/CAt | C/H | Changed missense |
| ADHFE1 | 8-67380528-TG-CA | TGt/CAt | C/H | Changed missense |
| KIAA1429 | 8-95504024-TG-CA | CAg/TGg | Q/W | Rescued nonsense |
| TAF2 | 8-120790304-TG-CA | aCA/aTG | T/M | Changed missense |
| ST3GAL1 | 8-134488007-GGT-AGC | AcC/GcT | T/A | Unchanged |
| COL22A1 | 8-139697478-TG-CA | cCA/cTG | P/L | Unchanged |
| SLC45A4 | 8-142222443-GTT-ATC | AaC/GaT | N/D | Unchanged |
| RHPN1 | 8-144462313-TG-CA | cTG/cCA | L/P | Unchanged |
| MROH6 | 8-144649625-TCG-CCA | CgA/TgG | R/W | Rescued nonsense |
| TIGD5 | 8-144680371-TGG-CGA | TgG/CgA | W/R | Rescued nonsense |
| PLEC | 8-144992361-TG-CA | cCA/cTG | P/L | Unchanged |
| GPAA1 | 8-145138621-TG-CA | gTG/gCA | V/A | Unchanged |
| CYHR1 | 8-145689915-GT-AC | cAC/cGT | H/R | Unchanged |
| MPDZ | 9-13219570-TG-CA | aCA/aTG | T/M | Changed missense |
| SLC24A2 | 9-19622268-TG-CA | cCA/cTG | P/L | Unchanged |
| FAM205A | 9-34724580-TG-CA | CAt/TGt | H/C | Changed missense |
| FAM205A | 9-34726418-TG-CA | cCA/cTG | P/L | Unchanged |
| OR13J1 | 9-35870000-GT-AC | cAC/cGT | H/R | Unchanged |
| OR13J1 | 9-35870001-TG-CA | CAc/TGc | H/C | Changed missense |
| FOXD4L6 | 9-69200917-GT-AC | cAC/cGT | H/R | Unchanged |
| FOXD4L5 | 9-70177441-GT-AC | tAC/tGT | Y/C | Unchanged |
| SMC5 | 9-72897440-TG-CA | TGt/CAt | C/H | Changed missense |
| DAPK1 | 9-90322023-GT-AC | aGT/aAC | S/N | Unchanged |
| PTCH1 | 9-98220526-GT-AC | aAC/aGT | N/S | Unchanged |
| GRIN3A | 9-104449294-GT-AC | aAC/aGT | N/S | Unchanged |
| OR13C5 | 9-107361449-TAG-CAA | CtA/TtG | L | Unchanged |
| AKAP2 | 9-112900199-TG-CA | tTG/tCA | L/S | Unchanged |
| PALM2-AKAP2 | 9-112900199-TG-CA | tTG/tCA | L/S | Unchanged |
| SVEP1 | 9-113169630-TG-CA | gCA/gTG | A/V | Unchanged |
| C9orf147 | 9-115249262-TG-CA | cCA/cTG | P/L | Unchanged |
| TNFSF8 | 9-117666499-GT-AC | tAC/tGT | Y/C | Unchanged |
| TNC | 9-117846580-TG-CA | CAg/TGg | Q/W | Rescued nonsense |
| TNC | 9-117848394-TG-CA | CAg/TGg | Q/W | Rescued nonsense |
| ASTN2 | 9-119495697-TG-CA | cCA/cTG | P/L | Unchanged |
| OR1B1 | 9-125391677-TG-CA | aCA/aTG | T/M | Changed missense |
| DENND1A | 9-126144758-TG-CA | tCA/tTG | S/L | Unchanged |
| LMX1B | 9-129455585-TCG-ACC | TcG/AcC | S/T | Unchanged |
| PPAPDC3 | 9-134183502-GT-AC | cGT/cAC | R/H | Unchanged |
| SURF6 | 9-136199466-GT-AC | ACg/GTg | T/V | Changed missense |
| FCN2 | 9-137775154-GT-AC | cGT/cAC | R/H | Unchanged |
| PPP1R26 | 9-138377657-TG-CA | aTG/aCA | M/T | Partially changed missense |
| OBP2A | 9-138440641-TG-CA | aTG/aCA | M/T | Partially changed missense |
| GLT6D1 | 9-138516189-GT-AC | cAC/cGT | H/R | Unchanged |
| QSOX2 | 9-139100805-TG-CA | cCA/cTG | P/L | Unchanged |
| CCDC183 | 9-139700605-TG-CA | TGg/CAg | W/Q | Rescued nonsense |
| ASMTL | X-1554649-TG-CA | aCA/aTG | T/M | Changed missense |
| FTHL17 | X-31089712-TG-CA | CAg/TGg | Q/W | Rescued nonsense |
| FAM47B | X-34961339-TTG-CTA | TtG/CtA | L | Unchanged |
| FAAH2 | X-57405162-GT-AC | cGT/cAC | R/H | Unchanged |
| ABCB7 | X-74284996-TG-CA | gCA/gTG | A/V | Unchanged |
| ZMAT1 | X-101138792-TG-CA | CAg/TGg | Q/W | Rescued nonsense |
| ATP1B4 | X-119500459-TG-CA | gTG/gCA | V/A | Unchanged |
| FAM122C | X-133941714-GT-AC | aGT/aAC | S/N | Unchanged |
| PLXNB3 | X-153041544-TG-CA | aTG/aCA | M/T | Partially changed missense |
| IL9R | X-155239682-GT-AC | GTg/ACg | V/T | Changed missense |

TABLE J
Specific targetable MNV mutations from the gnomAD database that can
be created with SPACE using Cas9 proteins with NGA PAM recognition.

| Gene_name | MNV | MNV_codons | MNV_AAs | MNV_category |
|---|---|---|---|---|
| SAMD11 | 1-877831-TG-CA | TGg/CAg | W/Q | Rescued nonsense |
| NOC2L | 1-888657-GAT-AAC | AtC/GtT | I/V | Unchanged |
| C1orf170 | 1-914874-GCT-ACC | AgC/GgT | S/G | Unchanged |
| CPSF3L | 1-1248279-GT-CA | gAC/gTG | D/V | Partially changed missense |
| CDK11A | 1-1636044-GT-AC | tAC/tGT | Y/C | Unchanged |
| PANK4 | 1-2441358-TG-CA | CAg/TGg | Q/W | Rescued nonsense |
| PRDM16 | 1-3328358-TCG-CCA | TcG/CcA | S/P | Unchanged |
| CCDC27 | 1-3677933-TG-CA | aTG/aCA | M/T | Partially changed missense |
| PER3 | 1-7870047-GT-AC | aGT/aAC | S/N | Unchanged |
| UTS2 | 1-7913430-GT-AC | ACg/GTg | T/V | Changed missense |
| C1orf127 | 1-11008695-TG-CA | cCA/cTG | P/L | Unchanged |
| TNFRSF1B | 1-12252927-GT-AC | GTg/ACg | V/T | Changed missense |
| PRAMEF10 | 1-12954806-TG-CA | aCA/aTG | T/M | Changed missense |
| PRAMEF10 | 1-12954852-TG-CA | CAc/TGc | H/C | Changed missense |
| PRAMEF26 | 1-13219066-TG-CA | CAc/TGc | H/C | Changed missense |
| PRAMEF13 | 1-13448351-TG-CA | CAc/TGc | H/C | Changed missense |
| PADI4 | 1-17657616-TG-CA | gTG/gCA | V/A | Unchanged |
| ARHGEF10L | 1-18023507-GAT-AAC | GaT/AaC | D/N | Unchanged |
| AKR7A3 | 1-19609253-GT-AC | ACt/GTt | T/V | Changed missense |
| PLA2G2C | 1-20490518-TG-CA | CAg/TGg | Q/W | Rescued nonsense |
| VWA5B1 | 1-20644156-TG-CA | TGt/CAt | C/H | Changed missense |
| ID3 | 1-23885497-GT-AC | ACt/GTt | T/V | Changed missense |
| CNR2 | 1-24201919-TTG-CTA | CaA/TaG | Q/* | Unchanged |
| CNR2 | 1-24201920-TG-CA | CAa/TGa | Q/* | Unchanged |
| WASF2 | 1-27736478-TG-CA | cCA/cTG | P/L | Unchanged |
| COL16A1 | 1-32158374-TG-CA | cCA/cTG | P/L | Unchanged |
| C1orf94 | 1-34677815-TG-CA | aTG/aCA | M/T | Partially changed missense |
| MOB3C | 1-47078637-TG-CA | gCA/gTG | A/V | Unchanged |
| PODN | 1-53544597-TG-CA | gTG/gCA | V/A | Unchanged |
| SLC1A7 | 1-53553754-TG-CA | CAg/TGg | Q/W | Rescued nonsense |
| KANK4 | 1-62738904-TG-CA | cCA/cTG | P/L | Unchanged |
| EFCAB7 | 1-63991265-GAT-AAC | GaT/AaC | D/N | Unchanged |
| IL23R | 1-67685387-TG-CA | cTG/cCA | L/P | Unchanged |
| SSX2IP | 1-85113203-TG-CA | gCA/gTG | A/V | Unchanged |
| TGFBR3 | 1-92262874-TG-CA | gCA/gTG | A/V | Unchanged |
| MTF2 | 1-93580580-TG-AC | TGc/ACc | C/T | Changed missense |
| ABCA4 | 1-94473845-TG-CA | cCA/cTG | P/L | Unchanged |
| GSTM5 | 1-110256333-GT-AC | GTt/ACt | V/T | Changed missense |
| GSTM5 | 1-110256333-GTT-ATC | GtT/AtC | V/I | Unchanged |
| IGSF3 | 1-117156584-TG-CA | CAg/TGg | Q/W | Rescued nonsense |
| NBPF14 | 1-148004782-GCT-ACC | AgC/GgT | S/G | Unchanged |
| HRNR | 1-152185789-GT-AC | cAC/cGT | H/R | Unchanged |
| HRNR | 1-152191750-TG-CA | tCA/tTG | S/L | Unchanged |
| HRNR | 1-152192978-TG-CA | CAa/TGa | Q/* | Unchanged |
| FLG | 1-152276391-TCG-CCA | CgA/TgG | R/W | Rescued nonsense |
| FLG | 1-152276827-TG-CA | CAg/TGg | Q/W | Rescued nonsense |
| FLG | 1-152277822-TG-CA | tCA/tTG | S/L | Unchanged |
| FLG | 1-152278335-TCG-CCA | CgA/TgG | R/W | Rescued nonsense |
| FLG | 1-152280372-GT-AC | cAC/cGT | H/R | Unchanged |
| FLG | 1-152285142-TCG-CCA | CgA/TgG | R/W | Rescued nonsense |
| IVL | 1-152882851-TG-CA | cTG/cCA | L/P | Unchanged |
| IQGAP3 | 1-156532977-TTG-CTA | CaA/TaG | Q/* | Unchanged |
| OR6N1 | 1-158735691-TG-CA | CAg/TGg | Q/W | Rescued nonsense |
| FCGR2C | 1-161565478-TG-CA | cTG/cCA | L/P | Unchanged |
| DCAF6 | 1-168013850-TG-CA | gTG/gCA | V/A | Unchanged |
| TNN | 1-175086123-TG-CA | aTG/aCA | M/T | Partially changed missense |
| ASTN1 | 1-176863867-TG-CA | CAc/TGc | H/C | Changed missense |
| RGS16 | 1-182569626-TG-CA | CAc/TGc | H/C | Changed missense |
| LAMC1 | 1-183105533-GT-AC | cGT/cAC | R/H | Unchanged |
| EDEM3 | 1-184677464-GAT-AAC | AtC/GtT | I/V | Unchanged |
| TARBP1 | 1-234569192-TG-CA | gCA/gTG | A/V | Unchanged |
| CNST | 1-246755124-TG-CA | tTG/tCA | L/S | Unchanged |
| OR2T33 | 1-248436965-TG-CA | CAc/TGc | H/C | Changed missense |
| OR2T12 | 1-248458729-TG-CA | CAc/TGc | H/C | Changed missense |
| OR2T29 | 1-248722722-TG-CA | CAa/TGa | Q/* | Unchanged |
| TAF3 | 10-8007560-TG-CA | gTG/gCA | V/A | Unchanged |
| NUDT5 | 10-12209752-TG-CA | gCA/gTG | A/V | Unchanged |
| CUBN | 10-16882518-TG-CA | aCA/aTG | T/M | Changed missense |
| C10ORF68 | 10-33137551-TG-CA | aTG/aCA | M/T | Partially changed missense |
| GPRIN2 | 10-47000004-TG-CA | gTG/gCA | V/A | Unchanged |
| PCDH15 | 10-55996608-TG-CA | cCA/cTG | P/L | Unchanged |
| HKDC1 | 10-71018660-TG-CA | TGg/CAg | W/Q | Rescued nonsense |
| HKDC1 | 10-71018660-TGG-CGA | TgG/CgA | W/R | Rescued nonsense |
| AC022532.1 | 10-72195439-TG-CA | gTG/gCA | V/A | Unchanged |
| NODAL | 10-72195439-TG-CA | CAc/TGc | H/C | Changed missense |
| ADAMTS14 | 10-72500763-TG-CA | cTG/cCA | L/P | Unchanged |
| KAT6B | 10-76789077-GT-AC | GTa/ACa | V/T | Changed missense |
| SFTPA1 | 10-81372092-TG-CA | aTG/aCA | M/T | Partially changed missense |
| LRIT1 | 10-85992511-TG-CA | cCA/cTG | P/L | Unchanged |
| CYP2C19 | 10-96534891-TG-CA | gTG/gCA | V/A | Unchanged |
| ABCC2 | 10-101563815-GT-AC | GTt/ACt | V/T | Changed missense |
| DNMBP | 10-101645498-TG-CA | cCA/cTG | P/L | Unchanged |
| NHLRC2 | 10-115636469-TG-CA | aTG/aCA | M/T | Partially changed missense |
| TACC2 | 10-123845322-TG-CA | TGg/CAg | W/Q | Rescued nonsense |
| PLEKHA1 | 10-124183731-GT-AC | cGT/cAC | R/H | Unchanged |
| C10orf90 | 10-128202435-TG-CA | gCA/gTG | A/V | Unchanged |
| ADAM8 | 10-135085754-GGT-AGC | AcC/GcT | T/A | Unchanged |
| TUBGCP2 | 10-135098672-GT-AC | tAC/tGT | Y/C | Unchanged |
| PIDD | 11-802379-TG-CA | CAa/TGa | Q/* | Unchanged |
| MUC6 | 11-1026093-TG-CA | cCA/cTG | P/L | Unchanged |
| MUC2 | 11-1081074-TG-CA | gTG/gCA | V/A | Unchanged |
| MUC2 | 11-1083291-GAT-AAC | GaT/AaC | D/N | Unchanged |
| MUC5B | 11-1264691-TG-CA | aTG/aCA | M/T | Partially changed missense |
| SYT8 | 11-1857173-TG-CA | TGc/CAc | C/H | Changed missense |
| SYT8 | 11-1857746-GT-AC | cGT/cAC | R/H | Unchanged |
| OR51F1 | 11-4790474-TG-CA | CAc/TGc | H/C | Changed missense |
| OR51B2 | 11-5344772-TG-CA | aCA/aTG | T/M | Changed missense |
| OR56A1 | 11-6048056-TG-CA | gCA/gTG | A/V | Unchanged |
| DNHD1 | 11-6520164-TG-AC | gTG/gAC | V/D | Changed missense |
| CYB5R2 | 11-7687713-GTT-ATC | AaC/GaT | N/D | Unchanged |
| TRIM66 | 11-8662090-TG-CA | CAt/TGt | H/C | Changed missense |
| TRIM66 | 11-8662516-TG-CA | CAc/TGc | H/C | Changed missense |
| USH1C | 11-17542439-TG-CA | cCA/cTG | P/L | Unchanged |
| SAAL1 | 11-18108492-TG-CA | aCA/aTG | T/M | Changed missense |
| MRGPRX3 | 11-18159455-TG-CA | TGg/CAg | W/Q | Rescued nonsense |
| RP11-113D6.6 | 11-18210480-TG-CA | cCA/cTG | P/L | Unchanged |
| MRGPRX1 | 11-18956305-GT-AC | gAC/gGT | D/G | Unchanged |
| WT1 | 11-32417945-TCG-CCA | CgA/TgG | R/W | Rescued nonsense |
| QSER1 | 11-32954344-GT-AC | GTa/ACa | V/T | Changed missense |
| ACCSL | 11-44080210-TG-CA | TGc/CAc | C/H | Changed missense |
| OR5D14 | 11-55563900-TG-CA | cTG/cCA | L/P | Unchanged |
| OR5L2 | 11-55595092-TG-CA | aTG/aCA | M/T | Partially changed missense |
| OR5AK2 | 11-56756663-TG-CA | aTG/aCA | M/T | Partially changed missense |
| SPTBN2 | 11-66481633-GT-AC | aAC/aGT | N/S | Unchanged |
| RHOD | 11-66837965-TG-CA | TGc/CAc | C/H | Changed missense |
| RAD9A | 11-67164789-GGT-AGC | GgT/AgC | G/S | Unchanged |
| FAM86C1 | 11-71504475-TG-CA | gTG/gCA | V/A | Unchanged |
| FAM86C1 | 11-71507186-TG-CA | TGt/CAt | C/H | Changed missense |
| DNAJB13 | 11-73681134-TG-CA | aTG/aCA | M/T | Partially changed missense |
| LRRC32 | 11-76372418-TG-CA | aCA/aTG | T/M | Changed missense |
| TTC12 | 11-113233196-TG-CA | gTG/gCA | V/A | Unchanged |
| TMPRSS5 | 11-113570405-TG-CA | gCA/gTG | A/V | Unchanged |
| MCAM | 11-119181170-TAG-CAA | CtA/TtG | L | Unchanged |
| OR8B3 | 11-124267189-TG-CA | CAt/TGt | H/C | Changed missense |
| AP000708.1 | 11-125366675-TGG-CGA | TgG/CgA | W/R | Rescued nonsense |
| HYLS1 | 11-125769354-TG-CA | TGt/CAt | C/H | Changed missense |
| RPUSD4 | 11-126081403-TG-CA | CAg/TGg | Q/W | Rescued nonsense |
| IQSEC3 | 12-266265-GT-AC | cGT/cAC | R/H | Unchanged |
| AKAP3 | 12-4737042-GT-AC | cAC/cGT | H/R | Unchanged |
| KCNA5 | 12-5154461-GT-AC | gGT/gAC | G/D | Unchanged |
| PLEKHG6 | 12-6421495-GCT-ACC | GcT/AcC | A/T | Unchanged |
| NCAPD2 | 12-6632471-GT-AC | GTg/ACg | V/T | Changed missense |
| TAS2R10 | 12-10978402-GT-AC | ACg/GTg | T/V | Changed missense |
| TAS2R20 | 12-11150319-TG-CA | gCA/gTG | A/V | Unchanged |
| TAS2R43 | 12-11244194-TG-CA | CAt/TGt | H/C | Changed missense |
| WBP11 | 12-14947554-TG-CA | CAg/TGg | Q/W | Rescued nonsense |
| ITPR2 | 12-26809316-GT-AC | cAC/cGT | H/R | Unchanged |
| LRRK2 | 12-40758652-TG-CA | aTG/aCA | M/T | Partially changed missense |
| OR8S1 | 12-48919659-TG-CA | cTG/cCA | L/P | Unchanged |
| OR8S1 | 12-48919793-TG-CA | TGc/CAc | C/H | Changed missense |
| KRT83 | 12-52708419-TG-CA | CAt/TGt | H/C | Changed missense |
| LGR5 | 12-71977787-TG-CA | gTG/gCA | V/A | Unchanged |
| ZFC3H1 | 12-72020103-GT-AC | gAC/gGT | D/G | Unchanged |
| C12orf50 | 12-88380094-TG-CA | CAg/TGg | Q/W | Rescued nonsense |
| TMCC3 | 12-94976084-TG-CA | gCA/gTG | A/V | Unchanged |
| GAS2L3 | 12-101005788-TG-CA | aTG/aCA | M/T | Partially changed missense |
| APPL2 | 12-105568176-GT-AC | gAC/gGT | D/G | Unchanged |
| SH2B3 | 12-111884608-TG-CA | TGg/CAg | W/Q | Rescued nonsense |
| TMEM233 | 12-120067559-GAT-AAC | GaT/AaC | D/N | Unchanged |
| RAB35 | 12-120535169-GT-AC | aAC/aGT | N/S | Unchanged |
| POLE | 12-133208979-TG-CA | tCA/tTG | S/L | Unchanged |
| GOLGA3 | 12-133389998-TG-CA | aCA/aTG | T/M | Changed missense |
| MIPEP | 13-24411770-GCT-ACC | AgC/GgT | S/G | Unchanged |
| LRRC63 | 13-46801793-TG-AC | TGt/ACt | C/T | Changed missense |
| LRRC63 | 13-46801794-GT-CA | tGT/tCA | C/S | Rescued nonsense |
| LMO7 | 13-76397731-GT-AC | GTt/ACt | V/T | Changed missense |
| LMO7 | 13-76423248-TG-CA | aTG/aCA | M/T | Partially changed missense |
| GPC6 | 13-95050781-GT-CA | GTg/CAg | V/Q | Changed missense |
| RNF113B | 13-98829176-TG-CA | cCA/cTG | P/L | Unchanged |
| TMEM255B | 13-114469204-GT-AC | GTt/ACt | V/T | Changed missense |
| SUPT16H | 14-21831052-TG-CA | cCA/cTG | P/L | Unchanged |
| RBM23 | 14-23374435-GAT-AAC | AtC/GtT | I/V | Unchanged |
| MIA2 | 14-39716965-TG-CA | aTG/aCA | M/T | Partially changed missense |
| NID2 | 14-52505517-GT-AC | gAC/gGT | D/G | Unchanged |
| FBXO34 | 14-55818706-TG-CA | cTG/cCA | L/P | Unchanged |
| TMEM260 | 14-57099859-GT-AC | aGT/aAC | S/N | Unchanged |
| KIAA0586 | 14-59010649-TG-CA | cTG/cCA | L/P | Unchanged |
| SYNE2 | 14-64491695-TG-CA | aTG/aCA | M/T | Partially changed missense |
| ADAM21 | 14-70924632-GT-AC | aGT/aAC | S/N | Unchanged |
| ELMSAN1 | 14-74196686-TG-CA | gCA/gTG | A/V | Unchanged |
| NGB | 14-77735616-TG-CA | CAg/TGg | Q/W | Rescued nonsense |
| ADCK1 | 14-78399633-TG-CA | TGg/CAg | W/Q | Rescued nonsense |
| GALC | 14-88411947-TG-CA | aCA/aTG | T/M | Changed missense |
| SERPINA6 | 14-94772429-GT-AC | gAC/gGT | D/G | Unchanged |
| CLMN | 14-95670567-TG-CA | tCA/tTG | S/L | Unchanged |
| SYNE3 | 14-95910933-TG-CA | gCA/gTG | A/V | Unchanged |
| SYNE3 | 14-95918625-TG-CA | cCA/cTG | P/L | Unchanged |
| WDR25 | 14-100847523-TG-CA | TGg/CAg | W/Q | Rescued nonsense |
| HSP90AA1 | 14-102568296-GGT-AGC | AcC/GcT | T/A | Unchanged |
| AHNAK2 | 14-105408315-TG-CA | cCA/cTG | P/L | Unchanged |
| AHNAK2 | 14-105414312-TG-CA | tCA/tTG | S/L | Unchanged |
| AHNAK2 | 14-105416619-GTT-ATC | AaC/GaT | N/D | Unchanged |
| IGHG1 | 14-106207843-TG-CA | aCA/aTG | T/M | Changed missense |
| IGHD1-14 | 14-106366499-GT-AC | ACc/GTc | T/V | Changed missense |
| IGHV3-7 | 14-106518472-GTT-ATC | AaC/GaT | N/D | Unchanged |
| IGHV1-8 | 14-106539268-GT-CA | aAC/aTG | N/M | Changed missense |
| IGHV3-15 | 14-106610520-GTT-ATC | AaC/GaT | N/D | Unchanged |
| IGHV3-23 | 14-106725402-GCT-ACC | AgC/GgT | S/G | Unchanged |
| IGHV1-24 | 14-106733264-TG-CA | aCA/aTG | T/M | Changed missense |
| IGHV4-39 | 14-106877682-GT-AC | aAC/aGT | N/S | Unchanged |
| IGHV1-69 | 14-107170054-TG-CA | aCA/aTG | T/M | Changed missense |
| IGHV2-70 | 14-107178886-GT-AC | aAC/aGT | N/S | Unchanged |
| POTEB2 | 15-21062000-TG-CA | aCA/aTG | T/M | Changed missense |
| GOLGA6L1 | 15-22742549-TG-CA | TGg/CAg | W/Q | Rescued nonsense |
| GOLGA6L2 | 15-23690466-TG-CA | tCA/tTG | S/L | Unchanged |
| NPAP1 | 15-24921468-TG-CA | TGg/CAg | W/Q | Rescued nonsense |
| GOLGA8M | 15-28949058-GT-AC | cAC/cGT | H/R | Unchanged |
| GOLGA8O | 15-32739268-GT-AC | cAC/cGT | H/R | Unchanged |
| PLA2G4F | 15-42434254-GTT-ATC | AaC/GaT | N/D | Unchanged |
| CDAN1 | 15-43023482-TG-CA | CAg/TGg | Q/W | Rescued nonsense |
| MYO5A | 15-52689526-TG-CA | aCA/aTG | T/M | Changed missense |
| FAM214A | 15-52903772-TG-CA | CAg/TGg | Q/W | Rescued nonsense |
| LDHAL6B | 15-59499627-TG-CA | gTG/gCA | V/A | Unchanged |
| MEGF11 | 15-66257408-TG-CA | CAc/TGc | H/C | Changed missense |
| IQCH | 15-67692501-GT-AC | cGT/cAC | R/H | Unchanged |
| ADPGK | 15-73067234-TG-CA | gCA/gTG | A/V | Unchanged |
| GOLGA6C | 15-75556026-TG-CA | aTG/aCA | M/T | Partially changed missense |
| GOLGA6D | 15-75582008-TG-CA | TGg/CAg | W/Q | Rescued nonsense |
| ADAMTS7 | 15-79058695-TG-CA | aCA/aTG | T/M | Changed missense |
| ADAMTS7 | 15-79069864-TG-CA | cCA/cTG | P/L | Unchanged |
| FAM154B | 15-82574879-TG-CA | TGg/CAg | W/Q | Rescued nonsense |
| FAM154B | 15-82574879-TGG-CGA | TgG/CgA | W/R | Rescued nonsense |
| AKAP13 | 15-86122779-TG-CA | TGg/CAg | W/Q | Rescued nonsense |
| CRTC3 | 15-91182001-GT-CA | gGT/gCA | G/A | Unchanged |
| SYNM | 15-99653800-TG-CA | gTG/gCA | V/A | Unchanged |
| WDR90 | 16-705360-TG-CA | gTG/gCA | V/A | Unchanged |
| WDR90 | 16-705844-TTG-CTA | TtG/CtA | L | Unchanged |
| TSC2 | 16-2138267-GAT-AAC | GaT/AaC | D/N | Unchanged |
| ABCA3 | 16-2334817-GAT-AAC | AtC/GtT | I/V | Unchanged |
| ZNF75A | 16-3367229-TG-CA | gTG/gCA | V/A | Unchanged |
| ADCY9 | 16-4165432-TGG-CGA | CcA/TcG | P/S | Unchanged |
| NAGPA | 16-5075633-GT-AC | ACt/GTt | T/V | Changed missense |
| CTD-3088G3.8 | 16-11540712-TG-CA | aCA/aTG | T/M | Changed missense |
| SDR42E2 | 16-22178256-GT-AC | cGT/cAC | R/H | Unchanged |
| GSG1L | 16-27974487-TG-CA | gCA/gTG | A/V | Unchanged |
| APOBR | 16-28508716-TG-CA | gTG/gCA | V/A | Unchanged |
| PRRT2 | 16-29825126-TTG-CTA | TtG/CtA | L | Unchanged |
| LPCAT2 | 16-55562465-TG-CA | aTG/aCA | M/T | Partially changed missense |
| MT4 | 16-56601722-TG-CA | TGg/CAg | W/Q | Rescued nonsense |
| MT1G | 16-56701267-GCT-ACC | AgC/GgT | S/G | Unchanged |
| COG3 | 16-69373414-TG-CA | aCA/aTG | T/M | Changed missense |
| NPIPB15 | 16-74425793-TCG-CCA | TcG/CcA | S/P | Unchanged |
| DNAAF1 | 16-84209738-TG-CA | tTG/tCA | L/S | Unchanged |
| ZCCHC14 | 16-87445363-GT-AC | cAC/cGT | H/R | Unchanged |
| PIEZO1 | 16-88791457-TG-CA | cCA/cTG | P/L | Unchanged |
| FANCA | 16-89866042-GT-AC | ACg/GTg | T/V | Changed missense |
| WDR81 | 17-1630991-TG-CA | cTG/cCA | L/P | Unchanged |
| SMYD4 | 17-1686409-GT-AC | tAC/tGT | Y/C | Unchanged |
| SMG6 | 17-2202943-TG-CA | tCA/tTG | S/L | Unchanged |
| MYBBP1A | 17-4455500-TG-CA | gCA/gTG | A/V | Unchanged |
| GLTPD2 | 17-4693170-TG-CA | gTG/gCA | V/A | Unchanged |
| ENO3 | 17-4856580-TG-CA | gTG/gCA | V/A | Unchanged |
| CAMTA2 | 17-4875566-TG-CA | cCA/cTG | P/L | Unchanged |
| AIPL1 | 17-6330068-TG-CA | cCA/cTG | P/L | Unchanged |
| KIAA0753 | 17-6493198-TG-CA | CAg/TGg | Q/W | Rescued nonsense |
| SLC35G6 | 17-7386279-TG-CA | TGg/CAg | W/Q | Rescued nonsense |
| PFAS | 17-8167600-TG-CA | cTG/cCA | L/P | Unchanged |
| CDRT1 | 17-15492409-TG-CA | gCA/gTG | A/V | Unchanged |
| EPN2 | 17-19232077-GT-AC | GTa/ACa | V/T | Changed missense |
| ULK2 | 17-19729495-TG-CA | gCA/gTG | A/V | Unchanged |
| NOS2 | 17-26115949-TG-CA | cCA/cTG | P/L | Unchanged |
| ABHD15 | 17-27889985-GT-AC | ACc/GTc | T/V | Changed missense |
| UTP6 | 17-30222002-TG-CA | CAg/TGg | Q/W | Rescued nonsense |
| SLFN14 | 17-33884804-TG-CA | CAg/TGg | Q/W | Rescued nonsense |
| LYZL6 | 17-34264862-TG-CA | gCA/gTG | A/V | Unchanged |
| FBXO47 | 17-37101380-TG-CA | CAa/TGa | Q/* | Unchanged |
| KRT28 | 17-38950272-TG-CA | aCA/aTG | T/M | Changed missense |
| KRTAP4-1 | 17-39340910-TG-CA | CAc/TGc | H/C | Changed missense |
| KRT32 | 17-39623363-TG-CA | CAg/TGg | Q/W | Rescued nonsense |
| NT5C3B | 17-39987130-TG-CA | gCA/gTG | A/V | Unchanged |
| MPP2 | 17-41960634-GT-CA | cAC/cTG | H/L | Partially changed missense |
| KIF18B | 17-43003484-TG-CA | gCA/gTG | A/V | Unchanged |
| MTMR4 | 17-56572524-TG-CA | aCA/aTG | T/M | Changed missense |
| C17orf47 | 17-56621359-TG-CA | tCA/tTG | S/L | Unchanged |
| TTYH2 | 17-72233559-GT-AC | GTt/ACt | V/T | Changed missense |
| TMEM104 | 17-72784932-GT-AC | GTg/ACg | V/T | Changed missense |
| QRICH2 | 17-74288262-TG-CA | CAt/TGt | H/C | Changed missense |
| TK1 | 17-76178748-TG-CA | gCA/gTG | A/V | Unchanged |
| DNAH17 | 17-76571086-GAT-AAC | AtC/GtT | I/V | Unchanged |
| C1QTNF1 | 17-77040127-GT-AC | cGT/cAC | R/H | Unchanged |
| AZI1 | 17-79173224-TG-CA | aCA/aTG | T/M | Changed missense |
| NARF | 17-80442721-GT-AC | cGT/cAC | R/H | Unchanged |
| TBCD | 17-80887242-GAT-AAC | GaT/AaC | D/N | Unchanged |
| LAMA1 | 18-6999628-TG-CA | tCA/tTG | S/L | Unchanged |
| PIEZO2 | 18-10763007-TG-CA | aCA/aTG | T/M | Changed missense |
| CEP192 | 18-13056682-GT-AC | GTg/ACg | V/T | Changed missense |
| CEP192 | 18-13095609-TG-CA | cTG/cCA | L/P | Unchanged |
| CTAGE1 | 18-19995753-TG-CA | cCA/cTG | P/L | Unchanged |
| NPC1 | 18-21140432-TG-CA | CAt/TGt | H/C | Changed missense |
| LAMA3 | 18-21413867-GCT-ACC | GcT/AcC | A/T | Unchanged |
| MOCOS | 18-33848581-TG-CA | gTG/gCA | V/A | Unchanged |
| FECH | 18-55221648-TG-CA | cCA/cTG | P/L | Unchanged |
| ALPK2 | 18-56204747-GT-AC | ACc/GTc | T/V | Changed missense |
| SERPINB3 | 18-61322973-TG-CA | CAt/TGt | H/C | Changed missense |
| ZNF516 | 18-74154999-GT-AC | aAC/aGT | N/S | Unchanged |
| LPPR3 | 19-813219-GT-AC | ACg/GTg | T/V | Changed missense |
| AP3D1 | 19-2111741-TGG-CGA | CcA/TcG | P/S | Unchanged |
| CACTIN | 19-3611972-GT-AC | aAC/aGT | N/S | Unchanged |
| TJP3 | 19-3750615-TG-CA | aTG/aCA | M/T | Partially changed missense |
| FSD1 | 19-4306310-GT-AC | cGT/cAC | R/H | Unchanged |
| PLIN4 | 19-4511381-GT-AC | ACc/GTc | T/V | Changed missense |
| ZNRF4 | 19-5455977-GT-AC | GTg/ACg | V/T | Changed missense |
| ZNRF4 | 19-5455978-TG-CA | gTG/gCA | V/A | Unchanged |
| LRRC8E | 19-7963976-TG-CA | aTG/aCA | M/T | Partially changed missense |
| ZNF558 | 19-8922729-TG-CA | CAt/TGt | H/C | Changed missense |
| MBD3L1 | 19-8953799-GTT-ATC | GtT/AtC | V/I | Unchanged |
| MUC16 | 19-9003645-GT-AC | ACt/GTt | T/V | Changed missense |
| MUC16 | 19-9048421-TG-CA | aCA/aTG | T/M | Changed missense |
| MUC16 | 19-9060812-TG-CA | tCA/tTG | S/L | Unchanged |
| MUC16 | 19-9065594-GGT-AGC | AcC/GcT | T/A | Unchanged |
| OR7G2 | 19-9213972-TG-CA | CAg/TGg | Q/W | Rescued nonsense |
| ZNF562 | 19-9764069-TG-CA | gCA/gTG | A/V | Unchanged |
| ZNF812 | 19-9801502-TG-CA | CAt/TGt | H/C | Changed missense |
| RDH8 | 19-10131999-TG-CA | aTG/aCA | M/T | Partially changed missense |
| DNMT1 | 19-10291181-TG-CA | CAt/TGt | H/C | Changed missense |
| ICAM3 | 19-10449512-GAT-AAC | AtC/GtT | I/V | Unchanged |
| ATG4D | 19-10659706-TG-CA | gTG/gCA | V/A | Unchanged |
| PRKCSH | 19-11553279-GCT-ACC | GcT/AcC | A/T | Unchanged |
| DNASE2 | 19-12989560-GCT-ACC | AgC/GgT | S/G | Unchanged |
| C19orf57 | 19-14000170-TG-CA | CAg/TGg | Q/W | Rescued nonsense |
| NOTCH3 | 19-15292437-TG-CA | cCA/cTG | P/L | Unchanged |
| CYP4F3 | 19-15763720-TG-CA | gTG/gCA | V/A | Unchanged |
| IL12RB1 | 19-18186618-TG-CA | CAg/TGg | Q/W | Rescued nonsense |
| COPE | 19-19011244-TG-CA | cCA/cTG | P/L | Unchanged |
| UQCRFS1 | 19-29704002-TG-CA | tCA/tTG | S/L | Unchanged |
| WDR88 | 19-33647379-TG-CA | TGt/CAt | C/H | Changed missense |
| GPR42 | 19-35863028-TG-CA | gTG/gCA | V/A | Unchanged |
| ZNF790 | 19-37310344-TG-CA | CAg/TGg | Q/W | Rescued nonsense |
| ZNF568 | 19-37441365-TG-CA | aTG/aCA | M/T | Partially changed missense |
| ZNF420 | 19-37619613-TTG-CTA | TtG/CtA | L | Unchanged |
| CAPN12 | 19-39226927-TG-CA | CAt/TGt | H/C | Changed missense |
| LGALS14 | 19-40197924-TG-CA | TGt/CAt | C/H | Changed missense |
| FCGBP | 19-40367862-TG-CA | cCA/cTG | P/L | Unchanged |
| FCGBP | 19-40367862-TGG-CGA | CcA/TcG | P/S | Unchanged |
| FCGBP | 19-40408053-TG-CA | cCA/cTG | P/L | Unchanged |
| ADCK4 | 19-41211056-TG-CA | CAc/TGc | H/C | Changed missense |
| CYP2A7 | 19-41384675-TG-CA | CAc/TGc | H/C | Changed missense |
| EXOSC5 | 19-41903220-GT-AC | ACg/GTg | T/V | Changed missense |
| PSG8 | 19-43269704-TG-CA | aCA/aTG | T/M | Changed missense |
| PSG4 | 19-43699424-TG-CA | cCA/cTG | P/L | Unchanged |
| PSG4 | 19-43709659-TG-CA | aCA/aTG | T/M | Changed missense |
| XRCC1 | 19-44055726-TG-CA | CAg/TGg | Q/W | Rescued nonsense |
| SRRM5 | 19-44117598-GT-AC | aGT/aAC | S/N | Unchanged |
| ZNF155 | 19-44500761-GT-AC | cGT/cAC | R/H | Unchanged |
| ZNF235 | 19-44792967-TAG-CAA | CtA/TtG | L | Unchanged |
| SPHK2 | 19-49132360-TG-CA | cTG/cCA | L/P | Unchanged |
| SIGLEC11 | 19-50461664-TG-CA | tCA/tTG | S/L | Unchanged |
| KLK14 | 19-51584951-TG-CA | CAg/TGg | Q/W | Rescued nonsense |
| SIGLEC9 | 19-51628481-TG-CA | TGg/CAg | W/Q | Rescued nonsense |
| ZNF577 | 19-52376119-GT-AC | ACt/GTt | T/V | Changed missense |
| ZNF615 | 19-52497745-TG-CA | CAg/TGg | Q/W | Rescued nonsense |
| ZNF468 | 19-53344426-TG-CA | CAt/TGt | H/C | Changed missense |
| ZNF331 | 19-54080067-TG-CA | TGt/CAt | C/H | Changed missense |
| LILRB3 | 19-54726226-TG-CA | gCA/gTG | A/V | Unchanged |
| LILRB2 | 19-54784130-TG-CA | CAc/TGc | H/C | Changed missense |
| NLRP4 | 19-56369591-GCT-ACC | GcT/AcC | A/T | Unchanged |
| ZSCAN5C | 19-56717604-GGT-AGC | GgT/AgC | G/S | Unchanged |
| ZNF667 | 19-56953863-TG-CA | aCA/aTG | T/M | Changed missense |
| ZNF749 | 19-57956186-TG-CA | gTG/gCA | V/A | Unchanged |
| APOB | 2-21232804-GT-AC | gAC/gGT | D/G | Unchanged |
| ADCY3 | 2-25046090-TG-CA | tCA/tTG | S/L | Unchanged |
| DRC1 | 2-26667615-TG-CA | TGg/CAg | W/Q | Rescued nonsense |
| CIB4 | 2-26804247-TG-CA | CAc/TGc | H/C | Changed missense |
| GCKR | 2-27730940-TG-CA | cTG/cCA | L/P | Unchanged |
| AC074091.13 | 2-27930502-GT-CA | aAC/aTG | N/M | Changed missense |
| PLB1 | 2-28865760-TTG-CTA | TtG/CtA | L | Unchanged |
| FAM179A | 2-29246044-TG-CA | gTG/gCA | V/A | Unchanged |
| LTBP1 | 2-33585796-TG-CA | gTG/gCA | V/A | Unchanged |
| ETAA1 | 2-67630476-TG-CA | aTG/aCA | M/T | Partially changed missense |
| ARHGAP25 | 2-69049941-TG-CA | aTG/aCA | M/T | Partially changed missense |
| REG3A | 2-79385786-TG-CA | aCA/aTG | T/M | Changed missense |
| CTNNA2 | 2-80801344-GTT-ATC | GtT/AtC | V/I | Unchanged |
| IGKV3-7 | 2-89278101-GCT-ACC | AgC/GgT | S/G | Unchanged |
| IGKV1-8 | 2-89292169-GT-AC | ACa/GTa | T/V | Changed missense |
| IGKV3-15 | 2-89384784-GAT-AAC | AtC/GtT | I/V | Unchanged |
| IGKV2D-28 | 2-89999461-GT-AC | aGT/aAC | S/N | Unchanged |
| ASTL | 2-96795857-TG-CA | CAg/TGg | Q/W | Rescued nonsense |
| TBC1D8 | 2-101656725-GT-AC | ACg/GTg | T/V | Changed missense |
| POTEF | 2-130832358-TG-CA | CAt/TGt | H/C | Changed missense |
| SMPD4 | 2-130910452-TG-CA | gCA/gTG | A/V | Unchanged |
| POTEJ | 2-131414431-TG-CA | TGc/CAc | C/H | Changed missense |
| LRP1B | 2-142567910-TG-CA | CAg/TGg | Q/W | Rescued nonsense |
| LY75 | 2-160639970-TG-CA | tCA/tTG | S/L | Unchanged |
| CD302 | 2-160639970-TG-CA | tCA/tTG | S/L | Unchanged |
| LY75-CD302 | 2-160639970-TG-CA | tCA/tTG | S/L | Unchanged |
| LY75 | 2-160711045-GT-CA | gAC/gTG | D/V | Partially changed missense |
| LY75-CD302 | 2-160711045-GT-CA | gAC/gTG | D/V | Partially changed missense |
| ABCB11 | 2-169789016-TG-CA | gCA/gTG | A/V | Unchanged |
| LRP2 | 2-169997051-GAT-AAC | AtC/GtT | I/V | Unchanged |
| TTN | 2-179430997-GT-AC | ACg/GTg | T/V | Changed missense |
| PLCL1 | 2-198950240-GT-AC | GTa/ACa | V/T | Changed missense |
| PIKFYVE | 2-209190330-TG-CA | tTG/tCA | L/S | Unchanged |
| ABCA12 | 2-215865575-TG-CA | cCA/cTG | P/L | Unchanged |
| CHPF | 2-220405321-TG-CA | CAg/TGg | Q/W | Rescued nonsense |
| OBSL1 | 2-220419236-TG-CA | aCA/aTG | T/M | Changed missense |
| IRS1 | 2-227661043-TG-CA | gCA/gTG | A/V | Unchanged |
| KCNJ13 | 2-233633459-TG-CA | aCA/aTG | T/M | Changed missense |
| UGT1A5 | 2-234621780-TG-CA | tTG/tCA | L/S | Unchanged |
| UGT1A5 | 2-234621810-TG-CA | gTG/gCA | V/A | Unchanged |
| SH3BP4 | 2-235950185-GAT-AAC | GaT/AaC | D/N | Unchanged |
| COL6A3 | 2-238243285-GT-AC | cAC/cGT | H/R | Unchanged |
| ESPNL | 2-239039637-TG-CA | gTG/gCA | V/A | Unchanged |
| PASK | 2-242066314-TG-CA | gCA/gTG | A/V | Unchanged |
| SIRPG | 20-1610894-GT-AC | tAC/tGT | Y/C | Unchanged |
| SIRPA | 20-1895796-TG-CA | tTG/tCA | L/S | Unchanged |
| SIRPA | 20-1896059-GT-AC | GTg/ACg | V/T | Changed missense |
| NOP56 | 20-2638579-TG-CA | aTG/aCA | M/T | Partially changed missense |
| PLCB1 | 20-8755241-GCT-ACC | GcT/AcC | A/T | Unchanged |
| CSTL1 | 20-23424613-TG-CA | TGg/CAg | W/Q | Rescued nonsense |
| CST3 | 20-23618395-TAG-CAA | CtA/TtG | L | Unchanged |
| ACSS1 | 20-25011423-TG-CA | gCA/gTG | A/V | Unchanged |
| PYGB | 20-25264812-GAT-AAC | GaT/AaC | D/N | Unchanged |
| ASXL1 | 20-31022959-TG-CA | cTG/cCA | L/P | Unchanged |
| KIAA1755 | 20-36841756-TG-CA | tCA/tTG | S/L | Unchanged |
| PREX1 | 20-47249170-GT-AC | aAC/aGT | N/S | Unchanged |
| TSHZ2 | 20-51870679-GT-AC | GTc/ACc | V/T | Changed missense |
| PCK1 | 20-56139402-GT-AC | gGT/gAC | G/D | Unchanged |
| LAMA5 | 20-60921641-GGT-AGC | AcC/GcT | T/A | Unchanged |
| HELZ2 | 20-62193445-GT-AC | ACg/GTg | T/V | Changed missense |
| GMEB2 | 20-62221610-TAG-CAA | CtA/TtG | L | Unchanged |
| ITSN1 | 21-35260480-GT-AC | cGT/cAC | R/H | Unchanged |
| FKSG68 | 21-37270507-TG-CA | CAg/TGg | Q/W | Rescued nonsense |
| KCNJ6 | 21-39086965-TG-CA | cCA/cTG | P/L | Unchanged |
| PSMG1 | 21-40549466-TG-CA | cCA/cTG | P/L | Unchanged |
| B3GALT5 | 21-41032740-TG-CA | aTG/aCA | M/T | Partially changed missense |
| KRTAP10-1 | 21-45959854-TG-CA | gCA/gTG | A/V | Unchanged |
| KRTAP10-3 | 21-45977994-GT-AC | ACg/GTg | T/V | Changed missense |
| KRTAP10-3 | 21-45978194-TG-CA | tCA/tTG | S/L | Unchanged |
| COL6A1 | 21-47410929-GGT-AGC | GgT/AgC | G/S | Unchanged |
| PCNT | 21-47787001-GT-AC | GTg/ACg | V/T | Changed missense |
| PCNT | 21-47787002-TG-CA | gTG/gCA | V/A | Unchanged |
| POM121L7 | 22-21481404-TG-CA | gCA/gTG | A/V | Unchanged |
| GGT2 | 22-21563319-TG-CA | gCA/gTG | A/V | Unchanged |
| IGLV7-46 | 22-22724394-TG-CA | tTG/tCA | L/S | Unchanged |
| IGLV3-1 | 22-23223435-GAT-AAC | GaT/AaC | D/N | Unchanged |
| ZNF70 | 22-24085990-TAG-CAA | CtA/TtG | L | Unchanged |
| CABIN1 | 22-24459437-GT-AC | tGT/tAC | C/Y | Unchanged |
| MYO18B | 22-26173661-TG-CA | TGg/CAg | W/Q | Rescued nonsense |
| C22orf31 | 22-29456733-TG-CA | tCA/tTG | S/L | Unchanged |
| SEC14L6 | 22-30934816-TG-CA | CAa/TGa | Q/* | Unchanged |
| OSBP2 | 22-31266544-GCT-ACC | GcT/AcC | A/T | Unchanged |
| C22orf23 | 22-38341134-TG-CA | aCA/aTG | T/M | Changed missense |
| APOBEC3F | 22-39448283-TG-CA | TGg/CAg | W/Q | Rescued nonsense |
| PARVB | 22-44489868-TG-CA | gTG/gCA | V/A | Unchanged |
| PRR5- | 22-45255629-GT-AC | cGT/cAC | R/H | Unchanged |
| ARHGAP8 | | | | |
| ARHGAP8 | 22-45255629-GT-AC | cGT/cAC | R/H | Unchanged |
| C22orf26 | 22-46447714-TG-CA | CAg/TGg | Q/W | Rescued nonsense |
| FLJ27365 | 22-46505786-TG-CA | TGg/CAg | W/Q | Rescued nonsense |
| CTA-299D3.8 | 22-48940551-GCT-ACC | AgC/GgT | S/G | Unchanged |
| MLC1 | 22-50515270-TG-CA | tCA/tTG | S/L | Unchanged |
| ATG7 | 3-11596300-GAT-AAC | GaT/AaC | D/N | Unchanged |
| TRANK1 | 3-36898715-GT-AC | ACc/GTc | T/V | Changed missense |
| XYLB | 3-38401838-GT-AC | gGT/gAC | G/D | Unchanged |
| XIRP1 | 3-39229900-TG-CA | CAg/TGg | Q/W | Rescued nonsense |
| KLHL40 | 3-42733468-TG-CA | TGc/CAc | C/H | Changed missense |
| TOPAZ1 | 3-44283672-TG-CA | TGc/CAc | C/H | Changed missense |
| BSN | 3-49693411-TG-CA | cTG/cCA | L/P | Unchanged |
| GMPPB | 3-49760039-TG-CA | CAg/TGg | Q/W | Rescued nonsense |
| CAMKV | 3-49897244-TG-AC | CAg/GTg | Q/V | Changed missense |
| STAB1 | 3-52555956-GT-AC | cGT/cAC | R/H | Unchanged |
| KBTBD8 | 3-67054649-TG-CA | TGt/CAt | C/H | Changed missense |
| EPHA3 | 3-89521693-TG-CA | TGg/CAg | W/Q | Rescued nonsense |
| CBLB | 3-105400420-TG-CA | cCA/cTG | P/L | Unchanged |
| MORC1 | 3-108690220-TG-CA | CAt/TGt | H/C | Changed missense |
| POPDC2 | 3-119367390-TG-CA | tCA/tTG | S/L | Unchanged |
| GOLGB1 | 3-121435621-TTG-CTA | CaA/TaG | Q/* | Unchanged |
| CD86 | 3-121825197-GTT-ATC | GtT/AtC | V/I | Unchanged |
| EFCAB12 | 3-129140489-TG-CA | aCA/aTG | T/M | Changed missense |
| FAIM | 3-138347988-TG-CA | tTG/tCA | L/S | Unchanged |
| HLTF | 3-148759325-TG-CA | gCA/gTG | A/V | Unchanged |
| P2RY13 | 3-151046308-GT-AC | ACg/GTg | T/V | Changed missense |
| IGSF10 | 3-151154666-TG-CA | aCA/aTG | T/M | Changed missense |
| GFM1 | 3-158366900-GT-AC | GTa/ACa | V/T | Changed missense |
| SI | 3-164767022-GT-CA | gAC/gTG | D/V | Partially changed missense |
| NAALADL2 | 3-174951756-TG-CA | aTG/aCA | M/T | Partially changed missense |
| LRRC15 | 3-194081152-GTT-ATC | AaC/GaT | N/D | Unchanged |
| MUC4 | 3-195505662-TGG-CGA | CcA/TcG | P/S | Unchanged |
| MUC4 | 3-195505848-TG-CA | gCA/gTG | A/V | Unchanged |
| MUC4 | 3-195507081-GGT-AGC | AcC/GcT | T/A | Unchanged |
| MUC4 | 3-195507096-TG-CA | gCA/gTG | A/V | Unchanged |
| MUC4 | 3-195507322-GT-AC | ACc/GTc | T/V | Changed missense |
| MUC4 | 3-195510168-TG-CA | gCA/gTG | A/V | Unchanged |
| MUC4 | 3-195510201-GGT-AGC | AcC/GcT | T/A | Unchanged |
| MUC4 | 3-195511512-TG-CA | gCA/gTG | A/V | Unchanged |
| MUC4 | 3-195511560-TG-CA | gCA/gTG | A/V | Unchanged |
| MUC4 | 3-195512187-TG-CA | tCA/tTG | S/L | Unchanged |
| MUC4 | 3-195512328-TG-CA | gCA/gTG | A/V | Unchanged |
| MUC4 | 3-195515385-GGT-AGC | AcC/GcT | T/A | Unchanged |
| MUC4 | 3-195515386-GT-AC | ACc/GTc | T/V | Changed missense |
| MUC4 | 3-195515482-GT-AC | ACc/GTc | T/V | Changed missense |
| HAUS3 | 4-2242083-TTG-ATC | CaA/GaT | Q/D | Changed missense |
| GRK4 | 4-3037213-TG-CA | cTG/cCA | L/P | Unchanged |
| GRK4 | 4-3039150-TG-CA | gTG/gCA | V/A | Unchanged |
| EVC2 | 4-5624669-GT-AC | ACg/GTg | T/V | Changed missense |
| USP17L10 | 4-9213568-GT-AC | GTa/ACa | V/T | Changed missense |
| PPARGC1A | 4-23815924-TG-CA | aCA/aTG | T/M | Changed missense |
| RFC1 | 4-39302029-TG-CA | cCA/cTG | P/L | Unchanged |
| KCTD8 | 4-44176960-TG-CA | aCA/aTG | T/M | Changed missense |
| COMMD8 | 4-47455085-TG-CA | gCA/gTG | A/V | Unchanged |
| NFXL1 | 4-47887536-TG-CA | gCA/gTG | A/V | Unchanged |
| NOA1 | 4-57829649-GT-AC | aAC/aGT | N/S | Unchanged |
| UGT2B28 | 4-70146310-GT-CA | gGT/gCA | G/A | Unchanged |
| UGT2B4 | 4-70355211-TG-CA | aCA/aTG | T/M | Changed missense |
| SMR3A | 4-71232430-TG-CA | TGt/CAt | C/H | Changed missense |
| HSD17B11 | 4-88258483-TG-CA | CAa/TGa | Q/* | Unchanged |
| DSPP | 4-88537403-GAT-AAC | GaT/AaC | D/N | Unchanged |
| SMARCAD1 | 4-95170839-GT-AC | aGT/aAC | S/N | Unchanged |
| TBCK | 4-107168430-TG-AC | CAa/GTa | Q/V | Changed missense |
| ANKRD50 | 4-125590478-TG-CA | cCA/cTG | P/L | Unchanged |
| PLRG1 | 4-155461113-TTG-CTA | CaA/TaG | Q/* | Unchanged |
| PALLD | 4-169433326-TG-CA | aTG/aCA | M/T | Partially changed missense |
| TENM3 | 4-183652173-GAT-AAC | GaT/AaC | D/N | Unchanged |
| SDHA | 5-228312-GAT-AAC | GaT/AaC | D/N | Unchanged |
| CCT5 | 5-10256160-GT-AC | cGT/cAC | R/H | Unchanged |
| DNAH5 | 5-13701525-TG-CA | aCA/aTG | T/M | Changed missense |
| DNAH5 | 5-13865980-TG-CA | aCA/aTG | T/M | Changed missense |
| EGFLAM | 5-38370537-TG-CA | TGg/CAg | W/Q | Rescued nonsense |
| MROH2B | 5-41057374-TG-CA | CAg/TGg | Q/W | Rescued nonsense |
| RP11-45H22.3 | 5-54252345-TG-CA | aCA/aTG | T/M | Changed missense |
| TNPO1 | 5-72183986-TG-CA | gTG/gCA | V/A | Unchanged |
| ARHGEF28 | 5-73072354-TG-CA | TGg/CAg | W/Q | Rescued nonsense |
| ENC1 | 5-73931246-TG-CA | tCA/tTG | S/L | Unchanged |
| HEXB | 5-73981270-TG-CA | tTG/tCA | L/S | Unchanged |
| F2RL1 | 5-76128521-GT-AC | aGT/aAC | S/N | Unchanged |
| PJA2 | 5-108713982-TG-CA | gCA/gTG | A/V | Unchanged |
| IRF1 | 5-131822055-TG-CA | cCA/cTG | P/L | Unchanged |
| WDR55 | 5-140048267-TG-CA | TGt/CAt | C/H | Changed missense |
| PCDHA1 | 5-140166017-GTT-ATC | GtT/AtC | V/I | Unchanged |
| PCDHA3 | 5-140181890-GCT-ACC | GcT/AcC | A/T | Unchanged |
| PCDHA11 | 5-140250252-TTG-CTA | TtG/CtA | L | Unchanged |
| PCDHA13 | 5-140263420-TTG-CTA | TtG/CtA | L | Unchanged |
| PCDHB3 | 5-140481840-GT-AC | cGT/cAC | R/H | Unchanged |
| PCDHB10 | 5-140574355-GCT-ACC | GcT/AcC | A/T | Unchanged |
| PCDHGA11 | 5-140802839-TG-CA | cTG/cCA | L/P | Unchanged |
| SPINK5 | 5-147510865-GT-AC | gGT/gAC | G/D | Unchanged |
| PDGFRB | 5-149495395-TG-CA | cCA/cTG | P/L | Unchanged |
| THG1L | 5-157164962-TG-CA | cTG/cCA | L/P | Unchanged |
| SH3PXD2B | 5-171789779-GCT-ACC | AgC/GgT | S/G | Unchanged |
| AC027309.1 | 5-172036332-TG-CA | aCA/aTG | T/M | Changed missense |
| AC136604.1 | 5-179078989-TG-CA | TGt/CAt | C/H | Changed missense |
| SLC22A23 | 6-3287118-TG-CA | tCA/tTG | S/L | Unchanged |
| MCUR1 | 6-13801614-GT-AC | ACt/GTt | T/V | Changed missense |
| GPLD1 | 6-24437445-GT-AC | ACa/GTa | T/V | Changed missense |
| GNL1 | 6-30515043-TG-CA | gCA/gTG | A/V | Unchanged |
| MUC22 | 6-30993738-TG-CA | aTG/aCA | M/T | Partially changed missense |
| MUC22 | 6-30994878-TG-CA | aTG/aCA | M/T | Partially changed missense |
| HLA-C | 6-31237771-GGT-AGC | AcC/GcT | T/A | Unchanged |
| HLA-B | 6-31324200-GCT-CCA | AgC/TgG | S/W | Changed missense |
| MICA | 6-31380194-TG-CA | TGt/CAt | C/H | Changed missense |
| PRRC2A | 6-31594626-GGT-CGA | GgT/CgA | G/R | Unchanged |
| HSPA1L | 6-31778272-GT-AC | ACg/GTg | T/V | Changed missense |
| NOTCH4 | 6-32188642-TG-CA | cCA/cTG | P/L | Unchanged |
| HLA-DRB5 | 6-32486437-TG-CA | CAg/TGg | Q/W | Rescued nonsense |
| HLA-DRB5 | 6-32489939-TG-CA | CAg/TGg | Q/W | Rescued nonsense |
| HLA-DQB1 | 6-32629129-TG-C A | CAg/TGg | Q/W | Rescued nonsense |
| WDR46 | 6-33256470-GT-AC | ACc/GTc | T/V | Changed missense |
| TAPBP | 6-33272972-GT-AC | ACt/GTt | T/V | Changed missense |
| TCP11 | 6-35086275-TG-CA | CAg/TGg | Q/W | Rescued nonsense |
| DNAH8 | 6-38976712-TG-CA | cTG/cCA | L/P | Unchanged |
| AARS2 | 6-44275009-GAT-AAC | AtC/GtT | I/V | Unchanged |
| SLC25A27 | 6-46623696-GCT-ACC | GcT/AcC | A/T | Unchanged |
| GPR115 | 6-47684573-TG-CA | aTG/aCA | M/T | Partially changed missense |
| GLYATL3 | 6-49479750-TG-CA | aTG/aCA | M/T | Partially changed missense |
| GFRAL | 6-55266625-TCG-CCA | TcG/CcA | S/P | Unchanged |
| BMP5 | 6-55639028-GT-AC | aAC/aGT | N/S | Unchanged |
| RP11-257K9.8 | 6-73935134-TG-AC | aCA/aGT | T/S | Changed missense |
| ZNF292 | 6-87969480-GT-AC | GTa/ACa | V/T | Changed missense |
| FAXC | 6-99771540-TG-CA | aCA/aTG | T/M | Changed missense |
| PPIL6 | 6-109752450-GT-AC | cAC/cGT | H/R | Unchanged |
| SLC22A16 | 6-110778128-TG-CA | CAt/TGt | H/C | Changed missense |
| TMEM200A | 6-130761796-GCT-ACC | GcT/AcC | A/T | Unchanged |
| CTAGE9 | 6-132030028-TG-CA | aCA/aTG | T/M | Changed missense |
| ULBP1 | 6-150290446-TG-CA | aTG/aCA | M/T | Partially changed missense |
| ESR1 | 6-152201874-GT-AC | cGT/cAC | R/H | Unchanged |
| SYNJ2 | 6-158497715-GTT-ATC | GtT/AtC | V/I | Unchanged |
| PLG | 6-161159625-GCT-ACC | GcT/AcC | A/T | Unchanged |
| PDE10A | 6-165895824-TG-CA | tCA/tTG | S/L | Unchanged |
| RPS6KA2 | 6-167271710-GT-AC | ACc/GTc | T/V | Changed missense |
| RP11-514O12.4 | 6-167271710-GT-AC | ACc/GTc | T/V | Changed missense |
| HEATR2 | 7-810218-GT-AC | GTg/ACg | V/T | Changed missense |
| CARD11 | 7-2946461-TCG-CCA | CgA/TgG | R/W | Rescued nonsense |
| STEAP1B | 7-22533235-TG-CA | CAt/TGt | H/C | Changed missense |
| SPDYE1 | 7-44047066-TG-CA | TGc/CAc | C/H | Changed missense |
| MYO1G | 7-45004063-TG-CA | CAg/TGg | Q/W | Rescued nonsense |
| MYO1G | 7-45009723-GT-AC | aAC/aGT | N/S | Unchanged |
| C7orf57 | 7-48086161-TG-CA | aTG/aCA | M/T | Partially changed missense |
| ZNF479 | 7-57188526-TG-CA | CAc/TGc | H/C | Changed missense |
| WBSCR28 | 7-73279482-TG-CA | TGg/CAg | W/Q | Rescued nonsense |
| C7orf63 | 7-89912301-GT-AC | GTg/ACg | V/T | Changed missense |
| MCM7 | 7-99693078-GAT-AAC | AtC/GtT | I/V | Unchanged |
| ZAN | 7-100377082-TG-CA | cTG/cCA | L/P | Unchanged |
| MUC12 | 7-100635940-TG-CA | aTG/aCA | M/T | Partially changed missense |
| MUC17 | 7-100684686-TG-CA | aTG/aCA | M/T | Partially changed missense |
| MYL10 | 7-101256833-TG-CA | gCA/gTG | A/V | Unchanged |
| FBXL13 | 7-102462636-TG-CA | gCA/gTG | A/V | Unchanged |
| NAMPT | 7-105903904-TG-CA | tCA/tTG | S/L | Unchanged |
| SLC13A1 | 7-122809233-GT-AC | aAC/aGT | N/S | Unchanged |
| STRIP2 | 7-129107227-TG-CA | TGc/CAc | C/H | Changed missense |
| PODXL | 7-131190696-TG-CA | gCA/gTG | A/V | Unchanged |
| NUP205 | 7-135269655-TG-CA | aTG/aCA | M/T | Partially changed missense |
| DGKI | 7-137150695-TG-CA | aCA/aTG | T/M | Changed missense |
| SVOPL | 7-138305806-TG-CA | gCA/gTG | A/V | Unchanged |
| KIAA1549 | 7-138596064-GT-AC | tAC/tGT | Y/C | Unchanged |
| TAS2R38 | 7-141672602-GAT-AAC | AtC/GtT | I/V | Unchanged |
| TRPV5 | 7-142625249-TG-CA | aCA/aTG | T/M | Changed missense |
| TAS2R60 | 7-143141474-GT-AC | cGT/cAC | R/H | Unchanged |
| ZNF212 | 7-148947470-TG-CA | aTG/aCA | M/T | Partially changed missense |
| RP1L1 | 8-10469340-GTT-ATC | AaC/GaT | N/D | Unchanged |
| CSGALNACT1 | 8-19316086-GT-AC | cAC/cGT | H/R | Unchanged |
| WRN | 8-31024654-TG-CA | TGt/CAt | C/H | Changed missense |
| ADHFE1 | 8-67380528-TG-CA | TGt/CAt | C/H | Changed missense |
| NBN | 8-90958422-TG-CA | cCA/cTG | P/L | Unchanged |
| KIAA1429 | 8-95504024-TG-CA | CAg/TGg | Q/W | Rescued nonsense |
| KIAA1429 | 8-95531419-TTG-CTA | CaA/TaG | Q/* | Unchanged |
| CCNE2 | 8-95894448-GT-AC | ACt/GTt | T/V | Changed missense |
| SPAG1 | 8-101252680-TG-CA | aTG/aCA | M/T | Partially changed missense |
| TAF2 | 8-120790304-TG-CA | aCA/aTG | T/M | Changed missense |
| TRMT12 | 8-125463250-TGG-CGA | TgG/CgA | W/R | Rescued nonsense |
| COL22A1 | 8-139697478-TG-CA | cCA/cTG | P/L | Unchanged |
| SLC45A4 | 8-142222443-GTT-ATC | AaC/GaT | N/D | Unchanged |
| LYPD2 | 8-143832509-GT-AC | aAC/aGT | N/S | Unchanged |
| RHPN1 | 8-144462313-TG-CA | cTG/cCA | L/P | Unchanged |
| MROH6 | 8-144649625-TCG-CCA | CgA/TgG | R/W | Rescued nonsense |
| DMRT3 | 9-990712-TTG-CTA | TtG/CtA | L | Unchanged |
| MPDZ | 9-13219570-TG-CA | aCA/aTG | T/M | Changed missense |
| IFNA14 | 9-21239650-GTT-ATC | AaC/GaT | N/D | Unchanged |
| PIGO | 9-35091950-GT-CA | ACa/TGa | T/* | Gained nonsense |
| OR13J1 | 9-35870001-TG-CA | CAc/TGc | H/C | Changed missense |
| PAX5 | 9-36840623-GT-AC | tAC/tGT | Y/C | Unchanged |
| ANKRD18A | 9-38575611-TG-CA | aCA/aTG | T/M | Changed missense |
| SMC5 | 9-72897440-TG-CA | TGt/CAt | C/H | Changed missense |
| PRUNE2 | 9-79324179-GT-AC | ACg/GTg | T/V | Changed missense |
| DAPK1 | 9-90322023-GT-AC | aGT/aAC | S/N | Unchanged |
| OR13C5 | 9-107361449-TAG-CAA | CtA/TtG | L | Unchanged |
| AKAP2 | 9-112900199-TG-CA | tTG/tCA | L/S | Unchanged |
| PALM2-AKAP2 | 9-112900199-TG-CA | tTG/tCA | L/S | Unchanged |
| C9orf84 | 9-114490308-TG-CA | CAt/TGt | H/C | Changed missense |
| TNFSF8 | 9-117666499-GT-AC | tAC/tGT | Y/C | Unchanged |
| TNC | 9-117804544-TG-CA | aCA/aTG | T/M | Changed missense |
| TNC | 9-117840328-GT-CA | aAC/aTG | N/M | Changed missense |
| TNC | 9-117848394-TG-CA | CAg/TGg | Q/W | Rescued nonsense |
| ASTN2 | 9-119495697-TG-CA | cCA/cTG | P/L | Unchanged |
| DENND1A | 9-126144758-TG-CA | tCA/tTG | S/L | Unchanged |
| LMX1B | 9-129455585-TCG-ACC | TcG/AcC | S/T | Unchanged |
| TBC1D13 | 9-131565554-TG-CA | gTG/gCA | V/A | Unchanged |
| PRRC2B | 9-134346314-GT-AC | cGT/cAC | R/H | Unchanged |
| PRRC2B | 9-134350647-TG-CA | aTG/aCA | M/T | Partially changed missense |
| OBP2A | 9-138440641-TG-CA | aTG/aCA | M/T | Partially changed missense |
| ABCA2 | 9-139903219-GAT-CAA | AtC/TtG | I/L | Changed missense |
| CSF2RA | X-1419454-TG-CA | gTG/gCA | V/A | Unchanged |
| ASMTL | X-1554649-TG-CA | aCA/aTG | T/M | Changed missense |
| HEPH | X-65382685-TG-CA | gTG/gCA | V/A | Unchanged |
| ABCB7 | X-74284996-TG-CA | gCA/gTG | A/V | Unchanged |
| ZMAT1 | X-101138792-TG-CA | CAg/TGg | Q/W | Rescued nonsense |
| ATP1B4 | X-119500459-TG-CA | gTG/gCA | V/A | Unchanged |
| USP26 | X-132160161-TG-CA | cCA/cTG | P/L | Unchanged |
| FAM122C | X-133941714-GT-AC | aGT/aAC | S/N | Unchanged |
| MAGEC3 | X-140985607-TG-CA | TGt/CAt | C/H | Changed missense |
| MAGEC1 | X-140993674-GTT-CTA | GtT/CtA | V/L | Unchanged |

TABLE K
Specific targetable MNV mutations from the gnomAD database that can
be created with SPACE using Cas9 proteins with NG PAM recognition.

| Gene_name | MNV | MNV_codons | MNV_AAs | MNV_category |
|---|---|---|---|---|
| SAMD11 | 1-877831-TG-CA | TGg/CAg | W/Q | Rescued nonsense |
| NOC2L | 1-888657-GAT-AAC | AtC/GtT | I/V | Unchanged |
| C1orf170 | 1-914874-GCT-ACC | AgC/GgT | S/G | Unchanged |
| C1orf170 | 1-914940-TG-CA | gCA/gTG | A/V | Unchanged |
| CPSF3L | 1-1247494-TG-CA | cCA/cTG | P/L | Unchanged |
| CPSF3L | 1-1248279-GT-CA | gAC/gTG | D/V | Partially changed missense |
| TAS1R3 | 1-1269554-TG-CA | TGc/CAc | C/H | Changed missense |
| DVL1 | 1-1277533-TG-CA | cCA/cTG | P/L | Unchanged |
| MIB2 | 1-1558792-TG-CA | aTG/aCA | M/T | Partially changed missense |
| CDK11A | 1-1636044-GT-AC | tAC/tGT | Y/C | Unchanged |
| PEX10 | 1-2340200-TG-CA | aCA/aTG | T/M | Changed missense |
| PANK4 | 1-2441358-TG-CA | CAg/TGg | Q/W | Rescued nonsense |
| PRDM16 | 1-3328358-TCG-CCA | TcG/CcA | S/P | Unchanged |
| WRAP73 | 1-3548136-TG-CA | gCA/gTG | A/V | Unchanged |
| CCDC27 | 1-3677933-TG-CA | aTG/aCA | M/T | Partially changed missense |
| PER3 | 1-7870047-GT-AC | aGT/aAC | S/N | Unchanged |
| PER3 | 1-7887493-TG-CA | cTG/cCA | L/P | Unchanged |
| UTS2 | 1-7913430-GT-AC | ACg/GTg | T/V | Changed missense |
| GPR157 | 1-9171348-TG-AC | CAc/GTc | H/V | Changed missense |
| PGD | 1-10473200-GAT-AAC | GaT/AaC | D/N | Unchanged |
| C1orf127 | 1-11008695-TG-CA | cCA/cTG | P/L | Unchanged |
| C1orf167 | 1-11836628-TG-CA | TGg/CAg | W/Q | Rescued nonsense |
| C1orf167 | 1-11839998-TG-CA | TGg/CAg | W/Q | Rescued nonsense |
| TNFRSF1B | 1-12252927-GT-AC | GTg/ACg | V/T | Changed missense |
| DHRS3 | 1-12638964-TG-CA | cCA/cTG | P/L | Unchanged |
| AADACL3 | 1-12779558-GAT-AAC | GaT/AaC | D/N | Unchanged |
| PRAMEF11 | 1-12884849-TG-CA | CAt/TGt | H/C | Changed missense |
| PRAMEF10 | 1-12954806-TG-CA | aCA/aTG | T/M | Changed missense |
| PRAMEF10 | 1-12954852-TG-CA | CAc/TGc | H/C | Changed missense |
| PRAMEF6 | 1-13002068-TG-CA | CAt/TGt | H/C | Changed missense |
| PRAMEF26 | 1-13219066-TG-CA | CAc/TGc | H/C | Changed missense |
| PRAMEF13 | 1-13448351-TG-CA | CAc/TGc | H/C | Changed missense |
| CASP9 | 1-15832543-TG-CA | CAg/TGg | Q/W | Rescued nonsense |
| SPEN | 1-16262717-GCT-ACC | GcT/AcC | A/T | Unchanged |
| CLCNKA | 1-16356531-GTT-ATC | GtT/AtC | V/I | Unchanged |
| CLCNKB | 1-16380196-TG-CA | aTG/aCA | M/T | Partially changed missense |
| PADI4 | 1-17657616-TG-CA | gTG/gCA | V/A | Unchanged |
| AC004824.2 | 1-17664280-TG-CA | gCA/gTG | A/V | Unchanged |
| ARHGEF10L | 1-17991052-TG-CA | TGg/CAg | W/Q | Rescued nonsense |
| ARHGEF10L | 1-18023507-GAT-AAC | GaT/AaC | D/N | Unchanged |
| TAS1R2 | 1-19166243-GAT-AAC | AtC/GtT | I/V | Unchanged |
| TAS1R2 | 1-19181391-GAT-AAC | AtC/GtT | I/V | Unchanged |
| ALDH4A1 | 1-19202917-TG-CA | tCA/tTG | S/L | Unchanged |
| AKR7A3 | 1-19609253-GT-AC | ACt/GTt | T/V | Changed missense |
| PLA2G2C | 1-20490518-TG-CA | CAg/TGg | Q/W | Rescued nonsense |
| VWA5B1 | 1-20644156-TG-CA | TGt/CAt | C/H | Changed missense |
| HSPG2 | 1-22149935-TG-CA | tCA/tTG | S/L | Unchanged |
| ID3 | 1-23885497-GT-AC | ACt/GTt | T/V | Changed missense |
| CNR2 | 1-24201919-TTG-CTA | CaA/TaG | Q/* | Unchanged |
| CNR2 | 1-24201920-TG-CA | CAa/TGa | Q/* | Unchanged |
| MYOM3 | 1-24417415-TG-CA | CAa/TGa | Q/* | Unchanged |
| SFN | 1-27189854-GT-CA | GTg/CAg | V/Q | Changed missense |
| C1orf172 | 1-27278573-TG-CA | CAg/TGg | Q/W | Rescued nonsense |
| FAM46B | 1-27332465-GT-AC | cAC/cGT | H/R | Unchanged |
| MAP3K6 | 1-27684006-TG-CA | CAc/TGc | H/C | Changed missense |
| WASF2 | 1-27736478-TG-CA | cCA/cTG | P/L | Unchanged |
| THEMIS2 | 1-28209360-GTT-ATC | GtT/AtC | V/I | Unchanged |
| MECR | 1-29529699-TG-CA | gCA/gTG | A/V | Unchanged |
| COL16A1 | 1-32158374-TG-CA | cCA/cTG | P/L | Unchanged |
| C1orf94 | 1-34677815-TG-CA | aTG/aCA | M/T | Partially changed missense |
| RP11-244H3.4 | 1-35476429-GT-CA | ACa/TGa | T/* | Gained nonsense |
| ZMYM6 | 1-35476429-GT-CA | ACa/TGa | T/* | Gained nonsense |
| HEYL | 1-40098328-TG-CA | CAc/TGc | H/C | Changed missense |
| COL9A2 | 1-40773149-TG-CA | CAg/TGg | Q/W | Rescued nonsense |
| ZFP69B | 1-40923018-TG-CA | TGt/CAt | C/H | Changed missense |
| HIVEP3 | 1-42048745-TG-CA | CAc/TGc | H/C | Changed missense |
| PTPRF | 1-44071220-TG-CA | gTG/gCA | V/A | Unchanged |
| MOB3C | 1-47078637-TG-CA | gCA/gTG | A/V | Unchanged |
| STIL | 1-47726087-TG-CA | CAt/TGt | H/C | Changed missense |
| PGDN | 1-53544597-TG-CA | gTG/gCA | V/A | Unchanged |
| SLC1A7 | 1-53553754-TG-CA | CAg/TGg | Q/W | Rescued nonsense |
| USP24 | 1-55638075-GT-AC | ACt/GTt | T/V | Changed missense |
| KANK4 | 1-62728838-TG-CA | CAc/TGc | H/C | Changed missense |
| KANK4 | 1-62738904-TG-CA | cCA/cTG | P/L | Unchanged |
| KANK4 | 1-62740446-TG-CA | cCA/cTG | P/L | Unchanged |
| EFCAB7 | 1-63991265-GAT-AAC | GaT/AaC | D/N | Unchanged |
| CACHD1 | 1-65113568-TG-CA | aTG/aCA | M/T | Partially changed missense |
| IL23R | 1-67685387-TG-CA | cTG/cCA | L/P | Unchanged |
| DEPDC1 | 1-68960349-TG-CA | gCA/gTG | A/V | Unchanged |
| LRRC53 | 1-74941293-GT-AC | ACt/GTt | T/V | Changed missense |
| SSX2IP | 1-85113203-TG-CA | gCA/gTG | A/V | Unchanged |
| DDAH1 | 1-85816134-TG-CA | gCA/gTG | A/V | Unchanged |
| COL24A1 | 1-86590915-GCT-ACC | AgC/GgT | S/G | Unchanged |
| GBP1 | 1-89525989-GT-AC | ACg/GTg | T/V | Changed missense |
| TGFBR3 | 1-92262874-TG-CA | gCA/gTG | A/V | Unchanged |
| MTF2 | 1-93580580-TG-AC | TGc/ACc | C/T | Changed missense |
| ABCA4 | 1-94473845-TG-CA | cCA/cTG | P/L | Unchanged |
| ABCA4 | 1-94544233-GT-AC | cAC/cGT | H/R | Unchanged |
| ABCA4 | 1-94544234-TG-CA | CAc/TGc | H/C | Changed missense |
| RWDD3 | 1-95709821-TG-CA | gTG/gCA | V/A | Unchanged |
| AKNAD1 | 1-109394564-TG-CA | gCA/gTG | A/V | Unchanged |
| GSTM5 | 1-110256333-GT-AC | GTt/ACt | V/T | Changed missense |
| GSTM5 | 1-110256333-GTT-ATC | GtT/AtC | V/I | Unchanged |
| EPS8L3 | 1-110295772-TG-CA | tCA/tTG | S/L | Unchanged |
| CSF1 | 1-110466466-TG-CA | cTG/cCA | L/P | Unchanged |
| IGSF3 | 1-117156584-TG-CA | CAg/TGg | Q/W | Rescued nonsense |
| SPAG17 | 1-118565952-TG-CA | cCA/cTG | P/L | Unchanged |
| WARS2 | 1-119584949-GT-AC | cAC/cGT | H/R | Unchanged |
| NBPF9 | 1-144811835-TG-CA | cTG/cCA | L/P | Unchanged |
| NBPF14 | 1-148004782-GCT-ACC | AgC/GgT | S/G | Unchanged |
| CTSK | 1-150771703-TG-CA | gCA/gTG | A/V | Unchanged |
| TCHH | 1-152083533-GT-AC | tAC/tGT | Y/C | Unchanged |
| HRNR | 1-152185789-GT-AC | cAC/cGT | H/R | Unchanged |
| HRNR | 1-152191750-TG-CA | tCA/tTG | S/L | Unchanged |
| HRNR | 1-152192978-TG-CA | CAa/TGa | Q/* | Unchanged |
| FLG | 1-152275725-TCG-CCA | CgA/TgG | R/W | Rescued nonsense |
| FLG | 1-152276391-TCG-CCA | CgA/TgG | R/W | Rescued nonsense |
| FLG | 1-152276430-GT-AC | cAC/cGT | H/R | Unchanged |
| FLG | 1-152276827-TG-CA | CAg/TGg | Q/W | Rescued nonsense |
| FLG | 1-152277822-TG-CA | tCA/tTG | S/L | Unchanged |
| FLG | 1-152278335-TCG-CCA | CgA/TgG | R/W | Rescued nonsense |
| FLG | 1-152280372-GT-AC | cAC/cGT | H/R | Unchanged |
| FLG | 1-152282925-GT-CA | cAC/cTG | H/L | Partially changed missense |
| FLG | 1-152285142-TCG-CCA | CgA/TgG | R/W | Rescued nonsense |
| FLG | 1-152285413-TG-CA | CAg/TGg | Q/W | Rescued nonsense |
| FLG | 1-152285512-TG-CA | CAg/TGg | Q/W | Rescued nonsense |
| IVL | 1-152882851-TG-CA | cTG/cCA | L/P | Unchanged |
| SPRR2D | 1-153012764-GT-AC | ACg/GTg | T/V | Changed missense |
| LOR | 1-153233519-GGT-AGC | GgT/AgC | G/S | Unchanged |
| RPS27 | 1-153964086-GT-CA | GTt/CAt | V/H | Changed missense |
| RXFP4 | 1-155912486-TG-CA | tTG/tCA | L/S | Unchanged |
| MEF2D | 1-156450740-GT-AC | aAC/aGT | N/S | Unchanged |
| IQGAP3 | 1-156532977-TTG-CTA | CaA/TaG | Q/* | Unchanged |
| SPTA1 | 1-158582646-TG-CA | gCA/gTG | A/V | Unchanged |
| OR6N1 | 1-158735691-TG-CA | CAg/TGg | Q/W | Rescued nonsense |
| C1orf204 | 1-159824966-TG-AC | CAa/GTa | Q/V | Changed missense |
| ADAMTS4 | 1-161163037-TG-CA | CAg/TGg | Q/W | Rescued nonsense |
| FCGR2C | 1-161565478-TG-CA | cTG/cCA | L/P | Unchanged |
| RXRG | 1-165398115-TG-CA | aCA/aTG | T/M | Changed missense |
| DCAF6 | 1-168013850-TG-CA | gTG/gCA | V/A | Unchanged |
| F5 | 1-169519049-TG-CA | CAa/TGa | Q/* | Unchanged |
| SELP | 1-169566308-GT-AC | aAC/aGT | N/S | Unchanged |
| TNN | 1-175086123-TG-CA | aTG/aCA | M/T | Partially changed missense |
| TNR | 1-175375683-TG-CA | aCA/aTG | T/M | Changed missense |
| ASTN1 | 1-176863867-TG-CA | CAc/TGc | H/C | Changed missense |
| SEC16B | 1-177929987-TG-CA | CAt/TGt | H/C | Changed missense |
| IER5 | 1-181058540-GT-AC | GTa/ACa | V/T | Changed missense |
| RGSL1 | 1-182443462-TTG-CTA | TtG/CtA | L | Unchanged |
| RGS16 | 1-182569625-GT-AC | cAC/cGT | H/R | Unchanged |
| RGS16 | 1-182569626-TG-CA | CAc/TGc | H/C | Changed missense |
| LAMC1 | 1-183094547-TG-CA | cTG/cCA | L/P | Unchanged |
| LAMC1 | 1-183105533-GT-AC | cGT/cAC | R/H | Unchanged |
| EDEM3 | 1-184677464-GAT-AAC | AtC/GtT | I/V | Unchanged |
| RNPEP | 1-201970560-GTT-ATC | GtT/AtC | V/I | Unchanged |
| PTPN7 | 1-202129826-TG-CA | CAg/TGg | Q/W | Rescued nonsense |
| LGR6 | 1-202287205-GT-AC | GTc/ACc | V/T | Changed missense |
| PLXNA2 | 1-208207839-GT-AC | tAC/tGT | Y/C | Unchanged |
| USH2A | 1-215848641-TG-CA | aCA/aTG | T/M | Changed missense |
| OBSCN | 1-228528298-TG-CA | gTG/gCA | V/A | Unchanged |
| TARBP1 | 1-234569192-TG-CA | gCA/gTG | A/V | Unchanged |
| HEATR1 | 1-236721731-TG-CA | gCA/gTG | A/V | Unchanged |
| HEATR1 | 1-236758887-TG-CA | CAt/TGt | H/C | Changed missense |
| CNST | 1-2467 55124-TG-CA | tTG/tCA | L/S | Unchanged |
| OR2W3 | 1-248059150-TG-CA | TGt/CAt | C/H | Changed missense |
| OR2W3 | 1-248059712-TG-CA | aTG/aCA | M/T | Partially changed missense |
| OR2T8 | 1-248084464-TG-CA | TGg/CAg | W/Q | Rescued nonsense |
| OR2L8 | 1-248112626-GT-AC | tGT/tAC | C/Y | Unchanged |
| OR2AK2 | 1-248129240-GT-AC | GTg/ACg | V/T | Changed missense |
| OR2M5 | 1-248308910-TG-CA | aTG/aCA | M/T | Partially changed missense |
| OR2M2 | 1-248343990-TG-CA | TGc/CAc | C/H | Changed missense |
| OR2T33 | 1-248436965-TG-CA | CAc/TGc | H/C | Changed missense |
| OR2T12 | 1-248458729-TG-CA | CAc/TGc | H/C | Changed missense |
| OR2T7 | 1-248604971-TG-CA | aTG/aCA | MT | Partially changed missense |
| OR2T29 | 1-248722722-TG-CA | CAa/TGa | Q/* | Unchanged |
| OR2T11 | 1-248789504-TG-CA | CAg/TGg | Q/W | Rescued nonsense |
| OR2T27 | 1-248813292-TG-CA | aCA/aTG | T/M | Changed missense |
| PITRM1 | 10-3180227-TG-CA | CAa/TGa | Q/* | Unchanged |
| TAF3 | 10-8007560-TG-CA | gTG/gCA | V/A | Unchanged |
| ECHDC3 | 10-11797434-GTT-ATC | GtT/AtC | V/I | Unchanged |
| NUDT5 | 10-12209752-TG-CA | gCA/gTG | A/V | Unchanged |
| DCLRE1C | 10-14974905-TG-CA | CAt/TGt | H/C | Changed missense |
| CUBN | 10-16882518-TG-CA | aCA/aTG | T/M | Changed missense |
| CUBN | 10-17024501-TGG-CGA | CcA/TcG | P/S | Unchanged |
| PLXDC2 | 10-20506418-GT-AC | GTa/ACa | V/T | Changed missense |
| KIAA1462 | 10-30316872-TG-CA | gCA/gTG | A/V | Unchanged |
| KIAA1462 | 10-30336682-TG-CA | cCA/cTG | P/L | Unchanged |
| C10ORF68 | 10-33137551-TG-CA | aTG/aCA | M/T | Partially changed missense |
| ZNF485 | 10-44111805-TG-CA | cTG/cCA | L/P | Unchanged |
| GPRIN2 | 10-47000004-TG-CA | gTG/gCA | V/A | Unchanged |
| FRMPD2 | 10-49380999-TG-CA | tCA/tTG | S/L | Unchanged |
| WDFY4 | 10-50109902-GT-AC | aGT/aAC | S/N | Unchanged |
| LRRC18 | 10-50121784-TG-CA | aCA/aTG | T/M | Changed missense |
| C10orf71 | 10-50533616-GT-AC | gGT/gAC | G/D | Unchanged |
| CHAT | 10-50856652-GT-AC | GTg/ACg | V/T | Changed missense |
| PCDH15 | 10-55996608-TG-CA | cCA/cTG | P/L | Unchanged |
| HKDC1 | 10-71018660-TG-CA | TGg/CAg | W/Q | Rescued nonsense |
| HKDC1 | 10-71018660-TGG-CGA | TgG/CgA | W/R | Rescued nonsense |
| AC022532.1 | 10-72195439-TG-CA | gTG/gCA | V/A | Unchanged |
| NODAL | 10-72195439-TG-CA | CAc/TGc | H/C | Changed missense |
| ADAMTS14 | 10-72500763-TG-CA | cTG/cCA | L/P | Unchanged |
| PLAU | 10-75673101-TG-CA | cTG/cCA | L/P | Unchanged |
| KAT6B | 10-76789077-GT-AC | GTa/ACa | V/T | Changed missense |
| DLG5 | 10-79616605-TG-CA | CAg/TGg | Q/W | Rescued nonsense |
| SFTPA1 | 10-81372092-TG-CA | aTG/aCA | M/T | Partially changed missense |
| NUTM2B | 10-81471741-TG-CA | TGg/CAg | W/Q | Rescued nonsense |
| LRIT1 | 10-85992511-TG-CA | cCA/cTG | P/L | Unchanged |
| PANK1 | 10-91344169-GT-AC | gAC/gGT | D/G | Unchanged |
| KIF20B | 10-91498127-TG-CA | TGt/CAt | C/H | Changed missense |
| CEP55 | 10-95276718-TG-CA | TGt/CAt | C/H | Changed missense |
| CYP2C19 | 10-96534891-TG-CA | gTG/gCA | V/A | Unchanged |
| SORBS1 | 10-97174351-GT-AC | ACg/GTg | T/V | Changed missense |
| HPS1 | 10-100179851-TG-CA | CAg/TGg | Q/W | Rescued nonsense |
| ABCC2 | 10-101563815-GT-AC | GTt/ACt | V/T | Changed missense |
| DNMBP | 10-101645498-TG-CA | cCA/cTG | P/L | Unchanged |
| DPCD | 10-103368654-TG-CA | tTG/tCA | L/S | Unchanged |
| CALHM1 | 10-105218359-TG-CA | gCA/gTG | A/V | Unchanged |
| NRAP | 10-115405615-TG-CA | CAg/TGg | Q/W | Rescued nonsense |
| NRAP | 10-115417289-GT-AC | cAC/cGT | H/R | Unchanged |
| NHLRC2 | 10-115636469-TG-CA | aTG/aCA | M/T | Partially changed missense |
| GFRA1 | 10-117884950-GT-AC | aAC/aGT | N/S | Unchanged |
| SFXN4 | 10-120919246-TG-CA | gCA/gTG | A/V | Unchanged |
| BAG3 | 10-121429633-TG-CA | TGt/CAt | C/H | Changed missense |
| TACC2 | 10-123845322-TG-CA | TGg/CAg | W/Q | Rescued nonsense |
| TACC2 | 10-123970530-TG-CA | gTG/gCA | V/A | Unchanged |
| PLEKHA1 | 10-124183731-GT-AC | cGT/cAC | R/H | Unchanged |
| DMBT1 | 10-124331845-TG-CA | tTG/tCA | L/S | Unchanged |
| DMBT1 | 10-124345741-TG-CA | tTG/tCA | L/S | Unchanged |
| CPXM2 | 10-125506302-TG-CA | CAg/TGg | Q/W | Rescued nonsense |
| CHST15 | 10-125798111-TG-CA | cCA/cTG | P/L | Unchanged |
| C10orf90 | 10-128202435-TG-CA | gCA/gTG | A/V | Unchanged |
| GPR123 | 10-134910553-GT-CA | GTc/CAc | V/H | Changed missense |
| KNDC1 | 10-135012162-TG-CA | cTG/cCA | L/P | Unchanged |
| ADAM8 | 10-135085754-GGT-AGC | AcC/GcT | T/A | Unchanged |
| TUBGCP2 | 10-135098672-GT-AC | tAC/tGT | Y/C | Unchanged |
| DRD4 | 11-640064-GT-AC | gGT/gAC | G/D | Unchanged |
| PIDD | 11-802379-TG-CA | CAa/TGa | Q/* | Unchanged |
| TSPAN4 | 11-862652-TTG-CTA | TtG/CtA | L | Unchanged |
| MUC6 | 11-1026093-TG-CA | cCA/cTG | P/L | Unchanged |
| MUC2 | 11-1081074-TG-CA | gTG/gCA | V/A | Unchanged |
| MUC2 | 11-1083291-GAT-AAC | GaT/AaC | D/N | Unchanged |
| MUC5AC | 11-1161315-TG-CA | gTG/gCA | V/A | Unchanged |
| MUC5B | 11-1264691-TG-CA | aTG/aCA | M/T | Partially changed missense |
| MUC5B | 11-1265936-TG-CA | cTG/cCA | L/P | Unchanged |
| KRTAP5-5 | 11-1651161-TG-CA | TGt/CAt | C/H | Changed missense |
| SYT8 | 11-1857173-TG-CA | TGc/CAc | C/H | Changed missense |
| SYT8 | 11-1857746-GT-AC | cGT/cAC | R/H | Unchanged |
| TRPM5 | 11-2432964-TG-CA | aCA/aTG | T/M | Changed missense |
| ART1 | 11-3681519-TG-CA | cTG/cCA | L/P | Unchanged |
| OR51F1 | 11-4790474-TG-CA | CAc/TGc | H/C | Changed missense |
| OR51G1 | 11-4944892-GGT-CGA | AcC/TcG | T/S | Unchanged |
| OR51B2 | 11-5344772-TG-CA | aCA/aTG | T/M | Changed missense |
| OR51B6 | 11-5373095-TG-CA | TGt/CAt | C/H | Changed missense |
| OR51J1 | 11-5424050-GT-AC | cGT/cAC | R/H | Unchanged |
| OR51I1 | 11-5462085-GT-AC | tAC/tGT | Y/C | Unchanged |
| OR56B1 | 11-5758062-TG-CA | TGc/CAc | C/H | Changed missense |
| OR56A3 | 11-5968728-TG-CA | aTG/aCA | M/T | Partially changed missense |
| OR56A1 | 11-6048056-TG-CA | gCA/gTG | A/V | Unchanged |
| OR56A1 | 11-6048515-TG-CA | cCA/cTG | P/L | Unchanged |
| DNHD1 | 11-6520164-TG-AC | gTG/gAC | V/D | Changed missense |
| DNHD1 | 11-6569896-GT-AC | GTg/ACg | V/T | Changed missense |
| RRP8 | 11-6622714-GT-AC | aAC/aGT | N/S | Unchanged |
| DCHS1 | 11-6655433-GT-AC | cAC/cGT | H/R | Unchanged |
| OR2AG2 | 11-6789862-GCT-ACC | AgC/GgT | S/G | Unchanged |
| OR10A5 | 11-6867355-GCT-ACC | GcT/AcC | A/T | Unchanged |
| CYB5R2 | 11-7687713-GTT-ATC | AaC/GaT | N/D | Unchanged |
| OVCH2 | 11-7712523-GT-AC | ACa/GTa | T/V | Changed missense |
| TRIM66 | 11-8662090-TG-CA | CAt/TGt | H/C | Changed missense |
| TRIM66 | 11-8662516-TG-CA | CAc/TGc | H/C | Changed missense |
| ADM | 11-10327257-GT-CA | GTt/CAt | V/H | Changed missense |
| GALNT18 | 11-11354346-TG-CA | gCA/gTG | A/V | Unchanged |
| KCNJ11 | 11-17408550-TG-CA | tCA/tTG | S/L | Unchanged |
| USH1C | 11-17542439-TG-CA | cCA/cTG | P/L | Unchanged |
| SAAL1 | 11-18108492-TG-CA | aCA/aTG | T/M | Changed missense |
| MRGPRX3 | 11-18159455-TG-CA | TGg/CAg | W/Q | Rescued nonsense |
| MRGPRX3 | 11-18159668-TG-CA | TGg/CAg | W/Q | Rescued nonsense |
| MRGPRX4 | 11-18195173-GTT-ATC | GtT/AtC | V/I | Unchanged |
| RP11-113D6.6 | 11-18210480-TG-CA | cCA/cTG | P/L | Unchanged |
| MRGPRX1 | 11-18956305-GT-AC | gAC/gGT | D/G | Unchanged |
| WT1 | 11-32417945-TCG-CCA | CgA/TgG | R/W | Rescued nonsense |
| QSER1 | 11-32954344-GT-AC | GTa/ACa | V/T | Changed missense |
| ACCSL | 11-44080210-TG-CA | TGc/CAc | C/H | Changed missense |
| ALX4 | 11-44297054-TG-CA | tCA/tTG | S/L | Unchanged |
| FOLH1 | 11-49186273-TG-CA | CAc/TGc | H/C | Changed missense |
| OR5D14 | 11-55563900-TG-CA | cTG/cCA | L/P | Unchanged |
| OR5L2 | 11-55595092-TG-CA | aTG/aCA | M/T | Partially changed missense |
| OR5AK2 | 11-56756663-TG-CA | aTG/aCA | M/T | Partially changed missense |
| PRG2 | 11-57155288-TG-CA | gCA/gTG | A/V | Unchanged |
| OR10Q1 | 11-57996084-GT-AC | aAC/aGT | N/S | Unchanged |
| RAB3IL1 | 11-61672235-TG-CA | aCA/aTG | T/M | Changed missense |
| ASRGL1 | 11-62105546-GT-CA | GTg/CAg | V/Q | Changed missense |
| RARRES3 | 11-63313642-GTT-ATC | GtT/AtC | V/I | Unchanged |
| CCDC88B | 11-64111928-TG-CA | TGg/CAg | W/Q | Rescued nonsense |
| CCDC88B | 11-64111928-TGG-CGA | TgG/CgA | W/R | Rescued nonsense |
| NAALADL1 | 11-64812791-TG-CA | aCA/aTG | T/M | Changed missense |
| EFEMP2 | 11-65635801-GTT-ATC | AaC/GaT | N/D | Unchanged |
| C11orf68 | 11-65685225-TG-CA | CAg/TGg | Q/W | Rescued nonsense |
| SPTBN2 | 11-66481633-GT-AC | aAC/aGT | N/S | Unchanged |
| RHOD | 11-66837965-TG-CA | TGc/CAc | C/H | Changed missense |
| RAD9A | 11-67164789-GGT-AGC | GgT/AgC | G/S | Unchanged |
| ALDH3B2 | 11-67432854-TG-CA | CAc/TGc | H/C | Changed missense |
| TPCN2 | 11-68851414-TG-CA | cTG/cCA | L/P | Unchanged |
| MYEOV | 11-69063393-TG-CA | gTG/gCA | V/A | Unchanged |
| KRTAP5-7 | 11-71238722-TG-CA | TGc/CAc | C/H | Changed missense |
| KRTAP5-8 | 11-71249162-TG-CA | TGt/CAt | C/H | Changed missense |
| KRTAP5-8 | 11-71249183-TG-CA | TGg/CAg | C/H | Changed missense |
| KRTAP5-10 | 11-71276715-TG-CA | TGt/CAt | C/H | Changed missense |
| KRTAP5-10 | 11-71276736-TG-CA | TGt/CAt | C/H | Changed missense |
| KRTAP5-10 | 11-71276946-GGT-AGC | GgT/AgC | G/S | Unchanged |
| KRTAP5-10 | 11-71276973-TG-CA | TGt/CAt | C/H | Changed missense |
| FAM86C1 | 11-71504475-TG-CA | gTG/gCA | V/A | Unchanged |
| FAM86C1 | 11-71507186-TG-CA | TGt/CAt | C/H | Changed missense |
| ARAP1 | 11-72415329-GT-AC | aAC/aGT | N/S | Unchanged |
| ARHGEF17 | 11-73022361-TG-CA | cTG/cCA | L/P | Unchanged |
| DNAJB13 | 11-73681134-TG-CA | aTG/aCA | M/T | Partially changed missense |
| LRRC32 | 11-76372418-TG-CA | aCA/aTG | T/M | Changed missense |
| MYO7A | 11-76853783-TG-CA | tTG/tCA | L/S | Unchanged |
| MMP1 | 11-102666316-TG-CA | gCA/gTG | A/V | Unchanged |
| TTC12 | 11-113233196-TG-CA | gTG/gCA | V/A | Unchanged |
| TMPRSS5 | 11-113570405-TG-CA | gCA/gTG | A/V | Unchanged |
| NXPE2 | 11-114568942-TG-CA | gTG/gCA | V/A | Unchanged |
| HYOU1 | 11-118919206-TG-CA | gCA/gTG | A/V | Unchanged |
| HYOU1 | 11-118925341-GT-AC | aAC/aGT | N/S | Unchanged |
| MCAM | 11-119181170-TAG-CAA | CtA/TtG | L | Unchanged |
| OR10G8 | 11-123900843-TG-CA | TGg/CAg | W/Q | Rescued nonsense |
| OR10G7 | 11-123909181-GT-AC | tAC/tGT | Y/C | Unchanged |
| OR10G7 | 11-123909194-TG-CA | CAg/TGg | Q/W | Rescued nonsense |
| OR8G5 | 11-124135008-TG-CA | TGt/CAt | C/H | Changed missense |
| OR8B3 | 11-124267177-TG-CA | CAg/TGg | Q/W | Rescued nonsense |
| OR8B3 | 11-124267189-TG-CA | CAt/TGt | H/C | Changed missense |
| AP000708.1 | 11-125366675-TGG-CGA | TgG/CgA | W/R | Rescued nonsense |
| HYLS1 | 11-125769354-TG-CA | TGt/CAt | C/H | Changed missense |
| RPUSD4 | 11-126081403-TG-CA | CAg/TGg | Q/W | Rescued nonsense |
| NFRKB | 11-129754668-TG-CA | aCA/aTG | T/M | Changed missense |
| IQSEC3 | 12-266265-GT-AC | cGT/cAC | R/H | Unchanged |
| IQSEC3 | 12-284056-GGT-AGC | GgT/AgC | G/S | Unchanged |
| SLC6A12 | 12-319111-TG-CA | gCA/gTG | A/V | Unchanged |
| AKAP3 | 12-4737042-GT-AC | cAC/cGT | H/R | Unchanged |
| KCNA5 | 12-5154461-GT-AC | gGT/gAC | G/D | Unchanged |
| VWF | 12-6094784-TG-CA | aCA/aTG | T/M | Changed missense |
| VWF | 12-6128441-GGT-AGC | AcC/GcT | T/A | Unchanged |
| VWF | 12-6131958-TG-CA | cCA/cTG | P/L | Unchanged |
| VWF | 12-6143984-TG-CA | CAg/TGg | Q/W | Rescued nonsense |
| PLEKHG6 | 12-6421495-GCT-ACC | GcT/AcC | A/T | Unchanged |
| SCNN1A | 12-6457061-GT-AC | ACc/GTc | T/V | Changed missense |
| NCAPD2 | 12-6632471-GT-AC | GTg/ACg | V/T | Changed missense |
| A2ML1 | 12-9010670-TG-CA | gTG/gCA | V/A | Unchanged |
| KLRC1 | 12-10603669-GT-AC | aAC/aGT | N/S | Unchanged |
| TAS2R10 | 12-10978402-GT-AC | ACg/GTg | T/V | Changed missense |
| TAS2R20 | 12-11150319-TG-CA | gCA/gTG | A/V | Unchanged |
| TAS2R43 | 12-11244194-TG-CA | CAt/TGt | H/C | Changed missense |
| WBP11 | 12-14947554-TG-CA | CAg/TGg | Q/W | Rescued nonsense |
| RERG | 12-15262179-GT-AC | aAC/aGT | N/S | Unchanged |
| SLC15A5 | 12-16377514-TG-CA | gCA/gTG | A/V | Unchanged |
| SLCO1B1 | 12-21331549-TG-CA | gTG/gCA | V/A | Unchanged |
| ITPR2 | 12-26809316-GT-AC | cAC/cGT | H/R | Unchanged |
| TSPAN11 | 12-31135579-TG-CA | gTG/gCA | V/A | Unchanged |
| DDX11 | 12-31256905-TG-CA | TGc/CAc | C/H | Changed missense |
| LRRK2 | 12-40758652-TG-CA | aTG/aCA | M/T | Partially changed missense |
| MUC19 | 12-40821867-GT-AC | GTc/ACc | V/T | Changed missense |
| MUC19 | 12-40838216-TG-CA | gTG/gCA | V/A | Unchanged |
| GXYLT1 | 12-42503467-TG-CA | aCA/aTG | T/M | Changed missense |
| OR8S1 | 12-48919659-TG-CA | cTG/cCA | L/P | Unchanged |
| OR8S1 | 12-48919793-TG-CA | TGc/CAc | C/H | Changed missense |
| KMT2D | 12-49425978-TG-CA | cCA/cTG | P/L | Unchanged |
| KRT83 | 12-52708419-TG-CA | CAt/TGt | H/C | Changed missense |
| KRT77 | 12-53096913-GGT-AGC | AcC/GcT | T/A | Unchanged |
| KRT4 | 12-53205757-TG-CA | CAg/TGg | Q/W | Rescued nonsense |
| ESPL1 | 12-53686703-GT-AC | cGT/cAC | R/H | Unchanged |
| SPRYD4 | 12-56862886-TG-CA | aTG/aCA | M/T | Partially changed missense |
| GPR182 | 12-57390038-TG-CA | TGc/CAc | C/H | Changed missense |
| METTL21B | 12-58174094-GT-CA | GTc/CAc | V/H | Changed missense |
| LRIG3 | 12-59281574-GT-CA | ACt/TGt | T/C | Changed missense |
| WIF1 | 12-65514266-TG-CA | gCA/gTG | A/V | Unchanged |
| MDM1 | 12-68720627-GT-AC | ACt/GTt | T/V | Changed missense |
| LGR5 | 12-71977787-TG-CA | gTG/gCA | V/A | Unchanged |
| ZFC3H1 | 12-72020103-GT-AC | gAC/gGT | D/G | Unchanged |
| C12orf50 | 12-88380094-TG-CA | CAg/TGg | Q/W | Rescued nonsense |
| CEP290 | 12-88505078-TG-CA | tCA/tTG | S/L | Unchanged |
| PLEKHG7 | 12-93150102-TG-CA | aTG/aCA | M/T | Partially changed missense |
| TMCC3 | 12-94976084-TG-CA | gCA/gTG | A/V | Unchanged |
| HAL | 12-96384249-TG-CA | cCA/cTG | P/L | Unchanged |
| ANKS1B | 12-99640557-TG-CA | cCA/cTG | P/L | Unchanged |
| GAS2L3 | 12-101005788-TG-CA | aTG/aCA | M/T | Partially changed missense |
| GNPTAB | 12-102158763-TG-CA | aCA/aTG | T/M | Changed missense |
| APPL2 | 12-105568176-GT-AC | gAC/gGT | D/G | Unchanged |
| SSH1 | 12-109182049-TG-CA | aCA/aTG | T/M | Changed missense |
| SH2B3 | 12-111884608-TG-CA | TGg/CAg | W/Q | Rescued nonsense |
| ATXN2 | 12-112036789-TG-CA | CAg/TGg | Q/W | Rescued nonsense |
| TMEM233 | 12-120067559-GAT-AAC | GaT/AaC | D/N | Unchanged |
| CIT | 12-120189888-TG-CA | aCA/aTG | T/M | Changed missense |
| RAB35 | 12-120535169-GT-AC | aAC/aGT | N/S | Unchanged |
| KDM2B | 12-121880520-GT-AC | gAC/gGT | D/G | Unchanged |
| HPD | 12-122295334-GT-AC | ACg/GTg | T/V | Changed missense |
| TMEM132C | 12-129153986-GT-AC | GTa/ACa | V/T | Changed missense |
| DDX51 | 12-132626668-TG-CA | CAg/TGg | Q/W | Rescued nonsense |
| POLE | 12-133208979-TG-CA | tCA/tTG | S/L | Unchanged |
| GOLGA3 | 12-133389998-TG-CA | aCA/aTG | T/M | Changed missense |
| MIPEP | 13-24411770-GCT-ACC | AgC/GgT | S/G | Unchanged |
| SHISA2 | 13-26620924-GT-AC | aAC/aGT | N/S | Unchanged |
| POSTN | 13-38160959-TG-CA | CAa/TGa | Q/* | Unchanged |
| COG3 | 13-46108853-TG-CA | tTG/tCA | L/S | Unchanged |
| LRRC63 | 13-46801793-TG-AC | TGt/ACt | C/T | Changed missense |
| LRRC63 | 13-46801794-GT-CA | tGT/tCA | C/S | Rescued nonsense |
| MLNR | 13-49795035-GT-AC | GTc/ACc | V/T | Changed missense |
| CAB39L | 13-49951193-TG-CA | cCA/cTG | P/L | Unchanged |
| AL445989.1 | 13-64321057-TG-CA | TGt/CAt | C/H | Changed missense |
| LMO7 | 13-76397731-GT-AC | GTt/ACt | V/T | Changed missense |
| LMO7 | 13-76423248-TG-CA | aTG/aCA | M/T | Partially changed missense |
| GPC6 | 13-95050781-GT-CA | GTg/CAg | V/Q | Changed missense |
| RNF113B | 13-98829176-TG-CA | cCA/cTG | P/L | Unchanged |
| DOCK9 | 13-99452659-GAT-CAA | AtC/TtG | I/L | Changed missense |
| TMTC4 | 13-101287365-TG-CA | gCA/gTG | A/V | Unchanged |
| ARHGEF7 | 13-111870035-GAT-AAC | GaT/AaC | D/N | Unchanged |
| ATP4B | 13-114309226-GT-AC | tAC/tGT | Y/C | Unchanged |
| TMEM255B | 13-114469204-GT-AC | GTt/ACt | V/T | Changed missense |
| OR11H6 | 14-20692643-TG-CA | TGt/CAt | C/H | Changed missense |
| SUPT16H | 14-21831052-TG-CA | cCA/cTG | P/L | Unchanged |
| OR10G2 | 14-22102592-TG-CA | CAc/TGc | H/C | Changed missense |
| OR4E2 | 14-22133648-GT-AC | GTg/ACg | V/T | Changed missense |
| TRAV8-6 | 14-22447168-TG-CA | gTG/gCA | V/A | Unchanged |
| RBM23 | 14-23374435-GAT-AAC | AtC/GtT | I/V | Unchanged |
| ACIN1 | 14-23530622-TG-CA | tCA/tTG | S/L | Unchanged |
| DHRS2 | 14-24114482-GT-AC | GTa/ACa | V/T | Changed missense |
| RABGGTA | 14-24736960-GT-AC | ACg/GTg | T/V | Changed missense |
| SDR39U1 | 14-24909362-TG-CA | CAg/TGg | Q/W | Rescued nonsense |
| MIA2 | 14-39716965-TG-CA | aTG/aCA | M/T | Partially changed missense |
| CTAGE5 | 14-39736679-GT-AC | GTt/ACt | V/T | Changed missense |
| PYGL | 14-51376774-GGT-AGC | AcC/GcT | T/A | Unchanged |
| NID2 | 14-52505517-GT-AC | gAC/gGT | D/G | Unchanged |
| FBXO34 | 14-55818706-TG-CA | cTG/cCA | L/P | Unchanged |
| TMEM260 | 14-57099859-GT-AC | aGT/aAC | S/N | Unchanged |
| KIAA0586 | 14-59010649-TG-CA | cTG/cCA | L/P | Unchanged |
| SYNE2 | 14-64491695-TG-CA | aTG/aCA | M/T | Partially changed missense |
| PLEKHH1 | 14-68053800-GCT-ACC | GcT/AcC | A/T | Unchanged |
| ADAM21 | 14-70924632-GT-AC | aGT/aAC | S/N | Unchanged |
| ADAM21 | 14-70925520-TG-CA | cTG/cCA | L/P | Unchanged |
| ELMSAN1 | 14-74196686-TG-CA | gCA/gTG | A/V | Unchanged |
| FLVCR2 | 14-76045857-TG-CA | gTG/gCA | V/A | Unchanged |
| C14orf166B | 14-77332408-TG-CA | gTG/gCA | V/A | Unchanged |
| NGB | 14-77735616-TG-CA | CAg/TGg | Q/W | Rescued nonsense |
| ADCK1 | 14-78399633-TG-CA | TGg/CAg | W/Q | Rescued nonsense |
| GALC | 14-88411947-TG-CA | aCA/aTG | T/M | Changed missense |
| SERPINA6 | 14-94772429-GT-AC | gAC/gGT | D/G | Unchanged |
| CLMN | 14-95670567-TG-CA | tCA/tTG | S/L | Unchanged |
| SYNE3 | 14-95910933-TG-CA | gCA/gTG | A/V | Unchanged |
| SYNE3 | 14-95912426-TG-CA | gCA/gTG | A/V | Unchanged |
| SYNE3 | 14-95918625-TG-CA | cCA/cTG | P/L | Unchanged |
| ATG2B | 14-96797724-GT-AC | cAC/cGT | H/R | Unchanged |
| WDR25 | 14-100847523-TG-CA | TGg/CAg | W/Q | Rescued nonsense |
| HSP90AA1 | 14-102568296-GGT-AGC | AcC/GcT | T/A | Unchanged |
| AHNAK2 | 14-105408315-TG-CA | cCA/cTG | P/L | Unchanged |
| AHNAK2 | 14-105413979-TG-CA | aCA/aTG | T/M | Changed missense |
| AHNAK2 | 14-105414132-TG-CA | cCA/cTG | P/L | Unchanged |
| AHNAK2 | 14-105414312-TG-CA | tCA/tTG | S/L | Unchanged |
| AHNAK2 | 14-105415353-GT-AC | gAC/gGT | D/G | Unchanged |
| AHNAK2 | 14-105415482-GT-AC | gAC/gGT | D/G | Unchanged |
| AHNAK2 | 14-105415959-TG-CA | aCA/aTG | T/M | Changed missense |
| AHNAK2 | 14-105416010-TG-CA | aCA/aTG | T/M | Changed missense |
| AHNAK2 | 14-105416154-TG-CA | gCA/gTG | A/V | Unchanged |
| AHNAK2 | 14-105416619-GTT-ATC | AaC/GaT | N/D | Unchanged |
| AHNAK2 | 14-105416787-TG-CA | tCA/tTG | S/L | Unchanged |
| AHNAK2 | 14-105416790-TG-CA | cCA/cTG | P/L | Unchanged |
| AHNAK2 | 14-105416872-TG-CA | CAg/TGg | Q/W | Rescued nonsense |
| AHNAK2 | 14-105416949-TG-CA | aCA/aTG | T/M | Changed missense |
| AHNAK2 | 14-105417102-TG-CA | cCA/cTG | P/L | Unchanged |
| AHNAK2 | 14-105417243-TG-CA | tCA/tTG | S/L | Unchanged |
| AHNAK2 | 14-105417444-TG-CA | aCA/aTG | T/M | Changed missense |
| AHNAK2 | 14-105417597-TG-CA | cCA/cTG | P/L | Unchanged |
| AHNAK2 | 14-105417862-TG-CA | CAg/TGg | Q/W | Rescued nonsense |
| AHNAK2 | 14-105418275-TG-CA | cCA/cTG | P/L | Unchanged |
| AHNAK2 | 14-105419424-TG-CA | aCA/aTG | T/M | Changed missense |
| AHNAK2 | 14-105419739-TG-CA | cCA/cTG | P/L | Unchanged |
| CDCA4 | 14-105477613-TG-CA | cCA/cTG | P/L | Unchanged |
| IGHE | 14-106066454-TG-CA | gCA/gTG | A/V | Unchanged |
| IGHG2 | 14-106110108-TG-CA | cCA/cTG | P/L | Unchanged |
| IGHG1 | 14-106207843-TG-CA | aCA/aTG | T/M | Changed missense |
| IGHG1 | 14-106209340-TG-CA | gCA/gTG | A/V | Unchanged |
| IGHG3 | 14-106237691-GT-AC | ACc/GTc | T/V | Changed missense |
| IGHM | 14-106322042-GT-AC | aAC/aGT | N/S | Unchanged |
| IGHJ5 | 14-106330035-GT-AC | ACc/GTc | T/V | Changed missense |
| IGHD1-14 | 14-106366499-GT-AC | ACc/GTc | T/V | Changed missense |
| IGHV1-2 | 14-106452734-GCT-ACC | AgC/GgT | S/G | Unchanged |
| IGHV3-7 | 14-106518472-GTT-ATC | AaC/GaT | N/D | Unchanged |
| IGHV1-8 | 14-106539268-GT-CA | aAC/aTG | N/M | Changed missense |
| IGHV3-15 | 14-106610344-GT-AC | ACg/GTc | T/V | Changed missense |
| IGHV3-15 | 14-106610520-GTT-ATC | AaC/GaT | N/D | Unchanged |
| IGHV1-18 | 14-106641680-GT-AC | aAC/aGT | N/S | Unchanged |
| IGHV3-23 | 14-106725402-GCT-ACC | AgC/GgT | S/G | Unchanged |
| IGHV1-24 | 14-106733264-TG-CA | aCA/aTG | T/M | Changed missense |
| IGHV2-26 | 14-106758084-TG-CA | aCA/aTG | T/M | Changed missense |
| IGHV4-39 | 14-106877682-GT-AC | aAC/aGT | N/S | Unchanged |
| IGHV3-49 | 14-107013058-TG-CA | aCA/aTG | T/M | Changed missense |
| IGHV4-61 | 14-107095294-TGG-AGC | CcA/GcT | P/A | Unchanged |
| IGHV1-69 | 14-107170054-TG-CA | aCA/aTG | T/M | Changed missense |
| IGHV2-70 | 14-107178886-GT-AC | aAC/aGT | N/S | Unchanged |
| IGHV3OR15-7 | 15-20192950-GT-AC | aAC/aGT | N/S | Unchanged |
| GOLGA6L6 | 15-20739885-TG-CA | CAg/TGg | Q/W | Rescued nonsense |
| POTEB2 | 15-21062000-TG-CA | aCA/aTG | T/M | Changed missense |
| GOLGA6L1 | 15-22742549-TG-CA | TGg/CAg | W/Q | Rescued nonsense |
| NIPA1 | 15-23052632-TG-CA | aCA/aTG | T/M | Changed missense |
| GOLGA8S | 15-23608316-GT-AC | cGT/cAC | R/H | Unchanged |
| GOLGA8S | 15-23610192-GT-AC | GTg/ACg | V/T | Changed missense |
| GOLGA6L2 | 15-23685252-TG-CA | gCA/gTG | A/V | Unchanged |
| GOLGA6L2 | 15-23690466-TG-CA | tCA/tTG | S/L | Unchanged |
| NPAP1 | 15-24921468-TG-CA | TGg/CAg | W/Q | Rescued nonsense |
| GABRA5 | 15-27182355-GTT-ATC | GtT/AtC | V/I | Unchanged |
| HERC2 | 15-28414665-TG-CA | tCA/tTG | S/L | Unchanged |
| HERC2 | 15-28419695-TG-CA | aCA/aTG | T/M | Changed missense |
| HERC2 | 15-28458960-TG-CA | cCA/cTG | P/L | Unchanged |
| GOLGA8M | 15-28949058-GT-AC | cAC/cGT | H/R | Unchanged |
| GOLGA8M | 15-28950750-TG-CA | CAg/TGg | Q/W | Rescued nonsense |
| APBA2 | 15-29347019-TG-CA | cTG/cCA | L/P | Unchanged |
| GOLGA8H | 15-30900873-TG-CA | TGc/CAc | C/H | Changed missense |
| GOLGA8O | 15-32739268-GT-AC | cAC/cGT | H/R | Unchanged |
| AVEN | 15-34159986-TG-CA | CAg/TGg | Q/W | Rescued nonsense |
| RPAP1 | 15-41821752-TCG-CCA | CgA/TgG | R/W | Rescued nonsense |
| SPTBN5 | 15-42178155-TG-CA | CAc/TGc | H/C | Changed missense |
| PLA2G4F | 15-42434254-GTT-ATC | AaC/GaT | N/D | Unchanged |
| CDAN1 | 15-43023482-TG-CA | CAg/TGg | Q/W | Rescued nonsense |
| MAP1A | 15-43817404-GAT-AAC | GaT/AaC | D/N | Unchanged |
| DUOX1 | 15-45444133-TG-CA | TGt/CAt | C/H | Changed missense |
| AP4E1 | 15-51217361-TG-CA | TGc/CAc | C/H | Changed missense |
| MYO5A | 15-52689526-TG-CA | aCA/aTG | T/M | Changed missense |
| FAM214A | 15-52903772-TG-CA | CAg/TGg | Q/W | Rescued nonsense |
| ADAM10 | 15-58971390-TG-CA | cCA/cTG | P/L | Unchanged |
| MYO1E | 15-59453384-TG-CA | gCA/gTG | A/V | Unchanged |
| LDHAL6B | 15-59499627-TG-CA | gTG/gCA | V/A | Unchanged |
| CILP | 15-65489688-TG-CA | CAg/TGg | Q/W | Rescued nonsense |
| IGDCC4 | 15-65676617-TG-CA | cCA/cTG | P/L | Unchanged |
| IGDCC4 | 15-65676617-TGG-CGA | CcA/TcG | P/S | Unchanged |
| MEGF11 | 15-66257408-TG-CA | CAc/TGc | H/C | Changed missense |
| IQCH | 15-67692501-GT-AC | cGT/cAC | R/H | Unchanged |
| SKOR1 | 15-68114498-TG-CA | cTG/cCA | L/P | Unchanged |
| ITGA11 | 15-68649587-GT-AC | aAC/aGT | N/S | Unchanged |
| ADPGK | 15-73067234-TG-CA | gCA/gTG | A/V | Unchanged |
| HCN4 | 15-73614834-TG-CA | cCA/cTG | P/L | Unchanged |
| C15orf39 | 15-75503147-GT-AC | gGT/gAC | G/D | Unchanged |
| GOLGA6C | 15-75556026-TG-CA | aTG/aCA | M/T | Partially changed missense |
| GOLGA6D | 15-75582008-TG-CA | TGg/CAg | W/Q | Rescued nonsense |
| CSPG4 | 15-75982185-TG-CA | cCA/cTG | P/L | Unchanged |
| ADAMTS7 | 15-79058695-TG-CA | aCA/aTG | T/M | Changed missense |
| ADAMTS7 | 15-79058951-TG-CA | CAt/TGt | H/C | Changed missense |
| ADAMTS7 | 15-79059182-TG-CA | CAc/TGc | H/C | Changed missense |
| ADAMTS7 | 15-79068536-TG-CA | CAg/TGg | Q/W | Rescued nonsense |
| ADAMTS7 | 15-79069864-TG-CA | cCA/cTG | P/L | Unchanged |
| ST20 | 15-80191342-TG-CA | cCA/cTG | P/L | Unchanged |
| FAM154B | 15-82574879-TG-CA | TGg/CAg | W/Q | Rescued nonsense |
| FAM154B | 15-82574879-TGG-CGA | TgG/CgA | W/R | Rescued nonsense |
| GOLGA6L10 | 15-82637334-TG-CA | CAt/TGt | H/C | Changed missense |
| GOLGA6L10 | 15-82637439-TG-CA | CAt/TGt | H/C | Changed missense |
| GOLGA6L18 | 15-82934933-TG-CA | CAt/TGt | H/C | Changed missense |
| GOLGA6L18 | 15-82934954-TG-CA | CAt/TGt | H/C | Changed missense |
| ADAMTSL3 | 15-84581903-GT-AC | cGT/cAC | R/H | Unchanged |
| WDR73 | 15-85188994-TG-CA | tCA/tTG | S/L | Unchanged |
| AKAP13 | 15-86122779-TG-CA | TGg/CAg | W/Q | Rescued nonsense |
| AKAP13 | 15-86124946-TG-CA | aTG/aCA | M/T | Partially changed missense |
| ACAN | 15-89392743-GCT-ACC | GcT/AcC | A/T | Unchanged |
| GDPGP1 | 15-90784250-TG-CA | aTG/aCA | M/T | Partially changed missense |
| CRTC3 | 15-91182001-GT-CA | gGT/gCA | G/A | Unchanged |
| SYNM | 15-99653800-TG-CA | gTG/gCA | V/A | Unchanged |
| CTD-2054N24.2 | 15-100348476-TG-CA | cTG/cCA | L/P | Unchanged |
| CERS3 | 15-100943044-GT-AC | gAC/gGT | D/G | Unchanged |
| TM2D3 | 15-102187076-GTT-ATC | AaC/GaT | N/D | Unchanged |
| RHBDF1 | 16-108585-TG-CA | tCA/tTG | S/L | Unchanged |
| PIGQ | 16-633125-TG-CA | TGt/CAt | C/H | Changed missense |
| PIGQ | 16-633353-TG-CA | TGt/CAt | C/H | Changed missense |
| WDR90 | 16-705360-TG-CA | gTG/gCA | V/A | Unchanged |
| WDR90 | 16-705844-TTG-CTA | TtG/CtA | L | Unchanged |
| WDR90 | 16-716273-TG-CA | TGc/CAc | C/H | Changed missense |
| CACNA1H | 16-1268540-TCG-CCA | TcG/CcA | S/P | Unchanged |
| TPSG1 | 16-1271938-TG-CA | tCA/tTG | S/L | Unchanged |
| TPSD1 | 16-1306354-GT-AC | GTg/ACg | V/T | Changed missense |
| TPSD1 | 16-1306355-TG-CA | gTG/gCA | V/A | Unchanged |
| SPSB3 | 16-1827836-TG-CA | tCA/tTG | S/L | Unchanged |
| ZNF598 | 16-2048704-TG-CA | CAg/TGg | Q/W | Rescued nonsense |
| ZNF598 | 16-2049849-TG-CA | aCA/aTG | T/M | Changed missense |
| TSC2 | 16-2138267-GAT-AAC | GaT/AaC | D/N | Unchanged |
| ABCA3 | 16-2334817-GAT-AAC | AtC/GtT | I/V | Unchanged |
| ZNF75A | 16-3367229-TG-CA | gTG/gCA | V/A | Unchanged |
| SLX4 | 16-3650991-TG-CA | cCA/cTG | P/L | Unchanged |
| ADCY9 | 16-4165432-TGG-CGA | CcA/TgG | P/S | Unchanged |
| NAGPA | 16-5075633-GT-AC | ACt/GTt | T/V | Changed missense |
| TEKT5 | 16-10769958-TG-CA | CAg/TGg | Q/W | Rescued nonsense |
| CTD-3088G3.8 | 16-11540712-TG-CA | aCA/aTG | T/M | Changed missense |
| ERCC4 | 16-14014069-TG-CA | cTG/cCA | L/P | Unchanged |
| SMG1 | 16-18839362-TG-CA | gCA/gTG | A/V | Unchanged |
| ACSM2B | 16-20570659-GAT-AAC | AtC/GtT | I/V | Unchanged |
| DNAH3 | 16-20963765-GGT-CGA | AcC/TcG | T/S | Unchanged |
| SDR42E2 | 16-22178256-GT-AC | cGT/cAC | R/H | Unchanged |
| PALB2 | 16-23646191-TG-CA | CAa/TGa | Q/* | Unchanged |
| ZKSCAN2 | 16-25263336-GT-CA | cAC/cTG | H/L | Partially changed missense |
| IL21R | 16-27460008-GT-CA | GTg/CAg | V/Q | Changed missense |
| GSG1L | 16-27974487-TG-CA | gCA/gTG | A/V | Unchanged |
| APOBR | 16-28508716-TG-CA | gTG/gCA | V/A | Unchanged |
| PRRT2 | 16-29825126-TTG-CTA | TtG/CtA | L | Unchanged |
| SEZ6L2 | 16-29883777-TAG-CAA | CtA/TtG | L | Unchanged |
| HSD3B7 | 16-30999461-GT-AC | cGT/cAC | R/H | Unchanged |
| ABCC11 | 16-48201432-TG-CA | CAc/TGc | H/C | Changed missense |
| SIAH1 | 16-48399356-GT-AC | tAC/tGT | Y/C | Unchanged |
| LPCAT2 | 16-55562465-TG-CA | aTG/aCA | M/T | Partially changed missense |
| CES5A | 16-55883618-TG-CA | cCA/cTG | P/L | Unchanged |
| MT4 | 16-56601722-TG-CA | TGg/CAg | W/Q | Rescued nonsense |
| MT1G | 16-56701267-GCT-ACC | AgC/GgT | S/G | Unchanged |
| SLC12A3 | 16-56904140-TG-CA | cTG/cCA | L/P | Unchanged |
| NLRC5 | 16-57060353-TG-CA | TGc/CAc | C/H | Changed missense |
| MMP15 | 16-58079216-GT-CA | GTg/CAg | V/Q | Changed missense |
| CCDC79 | 16-66792714-TG-CA | CAa/TGa | Q/* | Unchanged |
| E2F4 | 16-67228632-TG-CA | gTG/gCA | V/A | Unchanged |
| FHOD1 | 16-67266065-TG-CA | aCA/aTG | T/M | Changed missense |
| FAM65A | 16-67563735-TGG-CGA | TgG/CgA | W/R | Rescued nonsense |
| CTRL | 16-67964203-TG-CA | CAt/TGt | H/C | Changed missense |
| CDH1 | 16-68857439-GCT-ACC | GcT/AcC | A/T | Unchanged |
| COG8 | 16-69373414-TG-CA | aCA/aTG | T/M | Changed missense |
| CLEC18A | 16-69988319-TG-CA | cTG/cCA | L/P | Unchanged |
| CLEC18A | 16-69988372-GT-AC | GTg/ACg | V/T | Changed missense |
| CLEC18A | 16-69988373-TG-CA | gTG/gCA | V/A | Unchanged |
| NPIPB15 | 16-74425793-TCG-CCA | TcG/CcA | S/P | Unchanged |
| PLCG2 | 16-81819766-GCT-ACC | GcT/AcC | A/T | Unchanged |
| DNAAF1 | 16-84209738-TG-CA | tTG/tCA | L/S | Unchanged |
| TAF1C | 16-84215390-TG-CA | gCA/gTG | A/V | Unchanged |
| ZCCHC14 | 16-87445363-GT-AC | cAC/cGT | H/R | Unchanged |
| KLHDC4 | 16-87788864-GT-AC | ACc/GTc | T/V | Changed missense |
| PIEZO1 | 16-88783098-GAT-AAC | AtC/GtT | I/V | Unchanged |
| PIEZO1 | 16-88791457-TG-CA | cCA/cTG | P/L | Unchanged |
| PIEZO1 | 16-88807895-TG-CA | cCA/cTG | P/L | Unchanged |
| ACSF3 | 16-89167402-GAT-AAC | GaT/AaC | D/N | Unchanged |
| CDH15 | 16-89246668-GAT-AAC | GaT/AaC | D/N | Unchanged |
| ZNF276 | 16-89789898-TGG-CGA | TgG/CgA | W/R | Rescued nonsense |
| FANCA | 16-89866042-GT-AC | ACg/GTg | T/V | Changed missense |
| MYO1C | 17-1373518-TG-CA | CAg/TGg | Q/W | Rescued nonsense |
| WDR81 | 17-1630991-TG-CA | cTG/cCA | L/P | Unchanged |
| SMYD4 | 17-1686409-GT-AC | tAC/tGT | Y/C | Unchanged |
| SMG6 | 17-2202943-TG-CA | tCA/tTG | S/L | Unchanged |
| MNT | 17-2298615-TG-CA | cCA/cTG | P/L | Unchanged |
| ITGAE | 17-3657173-GAT-AAC | AtC/GtT | I/V | Unchanged |
| ZZEF1 | 17-3912208-GT-AC | ACg/GTg | T/V | Changed missense |
| ZZEF1 | 17-3953083-TG-CA | cCA/cTG | P/L | Unchanged |
| MYBBP1A | 17-4455500-TG-CA | gCA/gTG | A/V | Unchanged |
| CXCL16 | 17-4638484-TG-CA | cCA/cTG | P/L | Unchanged |
| GLTPD2 | 17-4693170-TG-CA | gTG/gCA | V/A | Unchanged |
| ENO3 | 17-4856580-TG-CA | gTG/gCA | V/A | Unchanged |
| CAMTA2 | 17-4875566-TG-CA | cCA/cTG | P/L | Unchanged |
| USP6 | 17-5042894-TG-CA | TGg/CAg | W/Q | Rescued nonsense |
| USP6 | 17-5042894-TGG-CGA | TgG/CgA | W/R | Rescued nonsense |
| RABEP1 | 17-5280439-GT-AC | cGT/cAC | R/H | Unchanged |
| AIPL1 | 17-6330068-TG-CA | cCA/cTG | P/L | Unchanged |
| AIPL1 | 17-6331803-TAG-CAA | CtA/TtG | L | Unchanged |
| KIAA0753 | 17-6493198-TG-CA | CAg/TGg | Q/W | Rescued nonsense |
| TEKT1 | 17-6733672-TG-CA | cCA/cTG | P/L | Unchanged |
| TEKT1 | 17-6733672-TGG-CGA | CcA/TcG | P/S | Unchanged |
| CLEC10A | 17-6979444-GT-CA | ACg/TGg | T/W | Changed missense |
| SLC35G6 | 17-7386279-TG-CA | TGg/CAg | W/Q | Rescued nonsense |
| KDM6B | 17-7750219-TG-CA | cTG/cCA | L/P | Unchanged |
| PER1 | 17-8048169-TG-CA | aCA/aTG | T/M | Changed missense |
| PFAS | 17-8167600-TG-CA | cTG/cCA | L/P | Unchanged |
| MYH13 | 17-10206595-TG-CA | CAc/TGc | H/C | Changed missense |
| MYH3 | 17-10543463-TG-CA | gCA/gTG | A/V | Unchanged |
| CDRT1 | 17-15492409-TG-CA | gCA/gTG | A/V | Unchanged |
| CDRT1 | 17-15510986-GTT-ATC | AaC/GaT | N/D | Unchanged |
| RP11-385D13.1 | 17-15510986-GTT-ATC | AaC/GaT | N/D | Unchanged |
| MYO15A | 17-18046898-TG-CA | TGc/CAc | C/H | Changed missense |
| EPN2 | 17-19232077-GT-AC | GTa/ACa | V/T | Changed missense |
| ULK2 | 17-19729495-TG-CA | gCA/gTG | A/V | Unchanged |
| NOS2 | 17-26115949-TG-CA | cCA/cTG | P/L | Unchanged |
| SEZ6 | 17-27286850-GT-AC | ACa/GTa | T/V | Changed missense |
| ABHD15 | 17-27889985-GT-AC | ACc/GTc | T/V | Changed missense |
| EVI2A | 17-29645505-TG-CA | CAg/TGg | Q/W | Rescued nonsense |
| CTD-2370N5.3 | 17-29645505-TG-CA | CAg/TGg | Q/W | Rescued nonsense |
| UTP6 | 17-30222002-TG-CA | CAg/TGg | Q/W | Rescued nonsense |
| SLFN11 | 17-33679633-TG-CA | gCA/gTG | A/V | Unchanged |
| SLFN14 | 17-33884804-TG-CA | CAg/TGg | Q/W | Rescued nonsense |
| LYZL6 | 17-34264862-TG-CA | gCA/gTG | A/V | Unchanged |
| FBXO47 | 17-37101380-TG-CA | CAa/TGa | Q/* | Unchanged |
| KRT28 | 17-38950272-TG-CA | aCA/aTG | T/M | Changed missense |
| KRT40 | 17-39135214-TTG-CTA | CaA/TaG | Q/* | Unchanged |
| KRTAP1-4 | 17-39186097-GT-AC | tAC/tGT | Y/C | Unchanged |
| KRTAP4-1 | 17-39340910-TG-CA | CAc/TGc | H/C | Changed missense |
| KRTAP9-3 | 17-39389025-GT-AC | gGT/gAC | G/D | Unchanged |
| KRT32 | 17-39619093-GT-AC | aAC/aGT | N/S | Unchanged |
| KRT32 | 17-39623363-TG-CA | CAg/TGg | Q/W | Rescued nonsense |
| KRT13 | 17-39659193-GT-AC | ACc/GTc | T/V | Changed missense |
| KRT16 | 17-39766679-TG-AC | CAg/GTg | Q/V | Changed missense |
| NT5C3B | 17-39987130-TG-CA | gCA/gTG | A/V | Unchanged |
| RUNDC1 | 17-41133071-TG-CA | TGg/CAg | W/Q | Rescued nonsense |
| MPP2 | 17-41960343-TG-CA | aCA/aTG | T/M | Changed missense |
| MPP2 | 17-41960634-GT-CA | cAC/cTG | H/L | Partially changed missense |
| KIF18B | 17-43003484-TG-CA | gCA/gTG | A/V | Unchanged |
| WNT9B | 17-44950122-TG-CA | aTG/aCA | M/T | Partially changed missense |
| B4GALNT2 | 17-47246163-TG-CA | TGc/CAc | C/H | Changed missense |
| COL1A1 | 17-48265493-GGT-AGC | AcC/GcT | T/A | Unchanged |
| RSAD1 | 17-48557348-TG-CA | tTG/tCA | L/S | Unchanged |
| BZRAP1 | 17-56396609-TG-CA | CAg/TGg | Q/W | Rescued nonsense |
| MTMR4 | 17-56572524-TG-CA | aCA/aTG | T/M | Changed missense |
| MTMR4 | 17-56584506-GCT-ACC | AgC/GgT | S/G | Unchanged |
| 44078 | 17-56598439-TG-CA | aCA/aTG | T/M | Changed missense |
| C17orf47 | 17-56621359-TG-CA | tCA/tTG | S/L | Unchanged |
| KCNH6 | 17-61611421-GAT-AAC | GaT/AaC | D/N | Unchanged |
| ARSG | 17-66364804-TG-CA | TGg/CAg | W/Q | Rescued nonsense |
| C17orf80 | 17-71232807-TG-CA | TGt/CAt | C/H | Changed missense |
| SDK2 | 17-71503636-TG-CA | cCA/cTG | P/L | Unchanged |
| TTYH2 | 17-72233559-GT-AC | GTt/ACt | V/T | Changed missense |
| NAT9 | 17-72769136-TG-CA | CAg/TGg | Q/W | Rescued nonsense |
| TMEM104 | 17-72784932-GT-AC | GTg/ACg | V/T | Changed missense |
| RECQL5 | 17-73625430-GT-AC | cAC/cGT | H/R | Unchanged |
| RECQL5 | 17-73625478-TG-CA | aCA/aTG | T/M | Changed missense |
| ITGB4 | 17-73753503-TG-CA | cTG/cCA | L/P | Unchanged |
| EVPL | 17-74014668-TG-CA | CAg/TGg | Q/W | Rescued nonsense |
| QRICH2 | 17-74287389-TG-CA | CAt/TGt | H/C | Changed missense |
| QRICH2 | 17-74288262-TG-CA | CAt/TGt | H/C | Changed missense |
| C17orf99 | 17-76157266-TG-CA | TGg/CAg | W/Q | Rescued nonsense |
| TK1 | 17-76178748-TG-CA | gCA/gTG | A/V | Unchanged |
| TMEM235 | 17-76230728-GT-AC | tGT/tAC | C/Y | Unchanged |
| DNAH17 | 17-76433897-GT-AC | cAC/cGT | H/R | Unchanged |
| DNAH17 | 17-76433898-TG-CA | CAc/TGc | H/C | Changed missense |
| DNAH17 | 17-76571086-GAT-AAC | AtC/GtT | I/V | Unchanged |
| C1QTNF1 | 17-77040127-GT-AC | cGT/cAC | R/H | Unchanged |
| ENPP7 | 17-77704912-TG-CA | cTG/cCA | L/P | Unchanged |
| ENPP7 | 17-77705043-GAT-AAC | GaT/AaC | D/N | Unchanged |
| CCDC40 | 17-78071050-GAT-AAC | GaT/AaC | D/N | Unchanged |
| RNF213 | 17-78263486-TG-CA | aTG/aCA | M/T | Partially changed missense |
| AZI1 | 17-79173224-TG-CA | aCA/aTG | T/M | Changed missense |
| SLC38A10 | 17-79220016-TG-CA | gCA/gTG | A/V | Unchanged |
| SLC38A10 | 17-79244802-TG-CA | aCA/aTG | T/M | Changed missense |
| TSPAN10 | 17-79612382-TG-CA | gTG/gCA | V/A | Unchanged |
| SLC25A10 | 17-79682557-GT-AC | cGT/cAC | R/H | Unchanged |
| PYCR1 | 17-79891147-TG-CA | tCA/tTG | S/L | Unchanged |
| ASPSCR1 | 17-79974729-GAT-AAC | GaT/AaC | D/N | Unchanged |
| NARF | 17-80442721-GT-AC | cGT/cAC | R/H | Unchanged |
| ZNF750 | 17-80788465-TG-CA | gCA/gTG | A/V | Unchanged |
| TBCD | 17-80863857-TG-CA | aTG/aCA | M/T | Partially changed missense |
| TBCD | 17-80887242-GAT-AAC | GaT/AaC | D/N | Unchanged |
| COLEC12 | 18-334758-TG-CA | cCA/cTG | P/L | Unchanged |
| LAMA1 | 18-6999628-TG-CA | tCA/tTG | S/L | Unchanged |
| PIEZO2 | 18-10763007-TG-CA | aCA/aTG | T/M | Changed missense |
| CEP192 | 18-13056682-GT-AC | GTg/ACg | V/T | Changed missense |
| CEP192 | 18-13095609-TG-CA | cTG/cCA | L/P | Unchanged |
| POTEC | 18-14543138-GT-AC | ACt/GTt | T/V | Changed missense |
| CTAGE1 | 18-19995753-TG-CA | cCA/cTG | P/L | Unchanged |
| NPC1 | 18-21119777-GT-AC | aAC/aGT | N/S | Unchanged |
| NPC1 | 18-21140432-TG-CA | CAt/TGt | H/C | Changed missense |
| LAMA3 | 18-21413867-GCT-ACC | GcT/AcC | A/T | Unchanged |
| MOCOS | 18-33848581-TG-CA | gTG/gCA | V/A | Unchanged |
| ZBTB7C | 18-45555829-TG-CA | aCA/aTG | T/M | Changed missense |
| MYO5B | 18-47500836-GT-AC | aAC/aGT | N/S | Unchanged |
| MYO5B | 18-47563297-GGT-AGC | AcC/GcT | T/A | Unchanged |
| MRO | 18-48331524-GT-AC | gAC/gGT | D/G | Unchanged |
| DYNAP | 18-52258547-GT-AC | GTt/ACt | V/T | Changed missense |
| FECH | 18-55221648-TG-CA | cCA/cTG | P/L | Unchanged |
| ALPK2 | 18-56204747-GT-AC | ACc/GTc | T/V | Changed missense |
| SERPINB4 | 18-61308176-GT-AC | ACt/GTt | T/V | Changed missense |
| SERPINB3 | 18-61322973-TG-CA | CAt/TGt | H/C | Changed missense |
| FAM69C | 18-72103782-TG-CA | CAa/TGa | Q/* | Unchanged |
| ZNF516 | 18-74154999-GT-AC | aAC/aGT | N/S | Unchanged |
| LPPR3 | 19-813219-GT-AC | ACg/GTg | T/V | Changed missense |
| GRIN3B | 19-1004740-TG-CA | TGg/CAg | W/Q | Rescued nonsense |
| C19orf26 | 19-1231141-GT-AC | cAC/cGT | H/R | Unchanged |
| MOB3A | 19-2078488-TG-CA | cCA/cTG | P/L | Unchanged |
| AP3D1 | 19-2111741-TGG-CGA | CcA/TcG | P/S | Unchanged |
| ZNF77 | 19-2934282-TG-CA | tCA/tTG | S/L | Unchanged |
| CACTIN | 19-3611972-GT-AC | aAC/aGT | N/S | Unchanged |
| PIP5K1C | 19-3641792-TG-CA | cCA/cTG | P/L | Unchanged |
| TJP3 | 19-3750615-TG-CA | aTG/aCA | M/T | Partially changed missense |
| FSD1 | 19-4306310-GT-AC | cGT/cAC | R/H | Unchanged |
| PLIN4 | 19-4511380-GGT-AGC | AcC/GcT | T/A | Unchanged |
| PLIN4 | 19-4511381-GT-AC | ACc/GTc | T/V | Changed missense |
| PLIN4 | 19-4511401-GAT-AAC | AtC/GtT | I/V | Unchanged |
| PLIN4 | 19-4511659-TG-CA | aCA/aTG | T/M | Changed missense |
| PLIN4 | 19-4512667-GGT-AGC | AcC/GcT | T/A | Unchanged |
| TICAM1 | 19-4818378-TG-CA | aCA/aTG | T/M | Changed missense |
| ZNRF4 | 19-5455977-GT-AC | GTg/ACg | V/T | Changed missense |
| ZNRF4 | 19-5455978-TG-CA | gTG/gCA | V/A | Unchanged |
| LONP1 | 19-5696146-GGT-AGC | AcC/GcT | T/A | Unchanged |
| PRR22 | 19-5783634-TG-CA | cCA/cTG | P/L | Unchanged |
| FUT6 | 19-5831724-TG-CA | cCA/cTG | P/L | Unchanged |
| FUT6 | 19-5832249-TCG-CCA | CgA/TgG | R/W | Rescued nonsense |
| FUT5 | 19-5866736-TG-CA | CAc/TGc | H/C | Changed missense |
| FUT5 | 19-5866816-GT-AC | gAC/gGT | D/G | Unchanged |
| FUT5 | 19-5866840-TG-CA | cCA/cTG | P/L | Unchanged |
| EMR1 | 19-6896483-GCT-ACC | GcT/AcC | A/T | Unchanged |
| EMR1 | 19-6926378-TG-CA | aTG/aCA | M/T | Partially changed missense |
| AC025278.1 | 19-7011826-TG-CA | TGg/CAg | W/Q | Rescued nonsense |
| STXBP2 | 19-7711219-GAT-AAC | GaT/AaC | D/N | Unchanged |
| LRRC8E | 19-7963976-TG-CA | aTG/aCA | M/T | Partially changed missense |
| FBN3 | 19-8181644-TG-CA | CAa/TGa | Q/* | Unchanged |
| ZNF558 | 19-8922729-TG-CA | CAt/TGt | H/C | Changed missense |
| MBD3L1 | 19-8953799-GTT-ATC | GtT/AtC | V/I | Unchanged |
| MUC16 | 19-9003645-GT-AC | ACt/GTt | T/V | Changed missense |
| MUC16 | 19-9048421-TG-CA | aCA/aTG | T/M | Changed missense |
| MUC16 | 19-9060812-TG-CA | tCA/tTG | S/L | Unchanged |
| MUC16 | 19-9065594-GGT-AGC | AcC/GcT | T/A | Unchanged |
| MUC16 | 19-9076082-TG-CA | aCA/aTG | T/M | Changed missense |
| OR7G2 | 19-9213972-TG-CA | CAg/TGg | Q/W | Rescued nonsense |
| ZNF562 | 19-9764069-TG-CA | gCA/gTG | A/V | Unchanged |
| ZNF812 | 19-9801502-TG-CA | CAt/TGt | H/C | Changed missense |
| ZNF812 | 19-9801717-TG-CA | gCA/gTG | A/V | Unchanged |
| RDH8 | 19-10131999-TG-CA | aTG/aCA | M/T | Partially changed missense |
| DNMT1 | 19-10265312-TG-CA | aCA/aTG | T/M | Changed missense |
| DNMT1 | 19-10291181-TG-CA | CAt/TGt | H/C | Changed missense |
| ICAM3 | 19-10449512-GAT-AAC | AtC/GtT | I/V | Unchanged |
| ATG4D | 19-10659706-TG-CA | gTG/gCA | V/A | Unchanged |
| KRI1 | 19-10676680-GT-AC | ACc/GTc | T/V | Changed missense |
| AP1M2 | 19-10692000-TG-CA | tCA/tTG | S/L | Unchanged |
| DOCK6 | 19-11319491-GT-AC | aAC/aGT | N/S | Unchanged |
| DOCK6 | 19-11319491-GTT-ATC | AaC/GaT | N/D | Unchanged |
| CCDC159 | 19-11465316-GT-CA | tGT/tCA | C/S | Rescued nonsense |
| PRKCSH | 19-11553279-GCT-ACC | GcT/AcC | A/T | Unchanged |
| RNASEH2A | 19-12921186-TG-CA | tTG/tCA | L/S | Unchanged |
| DNASE2 | 19-12989560-GCT-ACC | AgC/GgT | S/G | Unchanged |
| C19orf57 | 19-14000170-TG-CA | CAg/TGg | Q/W | Rescued nonsense |
| SAMD1 | 19-14199338-GT-AC | ACc/GTc | T/V | Changed missense |
| OR7A5 | 19-14938833-GT-AC | ACt/GTt | T/V | Changed missense |
| NOTCH3 | 19-15292437-TG-CA | cCA/cTG | P/L | Unchanged |
| NOTCH3 | 19-15302844-TG-CA | gCA/gTG | A/V | Unchanged |
| CYP4F3 | 19-15763720-TG-CA | gTG/gCA | V/A | Unchanged |
| CYP4F12 | 19-15793235-TG-CA | TGt/CAt | C/H | Changed missense |
| OR10H5 | 19-15905271-TG-CA | cTG/cCA | L/P | Unchanged |
| OR10H1 | 19-15918500-GGT-AGC | AcC/GcT | T/A | Unchanged |
| CHERP | 19-16633950-TG-CA | cCA/cTG | P/L | Unchanged |
| CPAMD8 | 19-17004049-TG-CA | CAg/TGg | Q/W | Rescued nonsense |
| MYO9B | 19-17316781-GT-AC | GTt/ACt | V/T | Changed missense |
| USE1 | 19-17330060-TG-CA | tTG/tCA | L/S | Unchanged |
| USHBP1 | 19-17366278-TG-CA | gCA/gTG | A/V | Unchanged |
| ANKLE1 | 19-17396548-GT-AC | gGT/gAC | G/D | Unchanged |
| ANO8 | 19-17435884-TG-CA | tCA/tTG | S/L | Unchanged |
| IL12RB1 | 19-18186618-TG-CA | CAg/TGg | Q/W | Rescued nonsense |
| KIAA1683 | 19-18375815-GT-AC | aAC/aGT | N/S | Unchanged |
| COPE | 19-19011244-TG-CA | cCA/cTG | P/L | Unchanged |
| ZNF729 | 19-22498942-GT-AC | tGT/tAC | C/Y | Unchanged |
| ZNF98 | 19-22574699-TG-CA | tCA/tTG | S/L | Unchanged |
| UQCRFS1 | 19-29704002-TG-CA | tCA/tTG | S/L | Unchanged |
| WDR88 | 19-33647379-TG-CA | TGt/CAt | C/H | Changed missense |
| LSR | 19-35741454-GTT-ATC | GtT/AtC | V/I | Unchanged |
| GPR42 | 19-35863028-TG-CA | gTG/gCA | V/A | Unchanged |
| APLP1 | 19-36370010-TG-CA | gTG/gCA | V/A | Unchanged |
| WDR62 | 19-36590329-TG-CA | tTG/tCA | L/S | Unchanged |
| ZNF260 | 19-37004938-TG-CA | tCA/tTG | S/L | Unchanged |
| ZNF790 | 19-37310344-TG-CA | CAg/TGg | Q/W | Rescued nonsense |
| ZNF568 | 19-37441365-TG-CA | aTG/aCA | M/T | Partially changed missense |
| ZNF420 | 19-37619613-TTG-CTA | TtG/CtA | L | Unchanged |
| ZNF585A | 19-37644051-TG-CA | gCA/gTG | A/V | Unchanged |
| ZNF607 | 19-38189616-TG-CA | aCA/aTG | T/M | Changed missense |
| CAPN12 | 19-39226927-TG-CA | CAt/TGt | H/C | Changed missense |
| DLL3 | 19-39994711-TG-CA | cTG/cCA | L/P | Unchanged |
| LGALS14 | 19-40197924-TG-CA | TGt/CAt | C/H | Changed missense |
| FCGBP | 19-40367862-TG-CA | cCA/cTG | P/L | Unchanged |
| FCGBP | 19-40367862-TGG-CGA | CcA/TcG | P/S | Unchanged |
| FCGBP | 19-40392359-TG-CA | gCA/gTG | A/V | Unchanged |
| FCGBP | 19-40408053-TG-CA | cCA/cTG | P/L | Unchanged |
| ADCK4 | 19-41211056-TG-CA | CAc/TGc | H/C | Changed missense |
| CYP2A7 | 19-41384675-TG-CA | CAc/TGc | H/C | Changed missense |
| EXOSC5 | 19-41903220-GT-AC | ACg/GTg | T/V | Changed missense |
| CEACAM6 | 19-42265243-GT-AC | GTt/ACt | V/T | Changed missense |
| DMRTC2 | 19-42354628-GT-AC | gGT/gAC | G/D | Unchanged |
| PSG8 | 19-43269704-TG-CA | aCA/aTG | T/M | Changed missense |
| PSG11 | 19-43519440-GTT-ATC | AaC/GaT | N/D | Unchanged |
| PSG4 | 19-43699424-TG-CA | cCA/cTG | P/L | Unchanged |
| PSG4 | 19-43709659-TG-CA | aCA/aTG | T/M | Changed missense |
| XRCC1 | 19-44055726-TG-CA | CAg/TGg | Q/W | Rescued nonsense |
| SRRM5 | 19-44117598-GT-AC | aGT/aAC | S/N | Unchanged |
| ZNF221 | 19-44470420-TG-CA | TGt/CAt | C/H | Changed missense |
| ZNF155 | 19-44500761-GT-AC | cGT/cAC | R/H | Unchanged |
| ZNF235 | 19-44792967-TAG-CAA | CtA/TtG | L | Unchanged |
| ZNF180 | 19-44981832-TG-CA | CAt/TGt | H/C | Changed missense |
| APOC4 | 19-45448036-TG-CA | cTG/cCA | L/P | Unchanged |
| APOC4-APOC2 | 19-45448036-TG-CA | cTG/cCA | L/P | Unchanged |
| CLPTM1 | 19-45490635-GT-AC | gGT/gAC | G/D | Unchanged |
| HIF3A | 19-46815497-GAT-AAC | GaT/AaC | D/N | Unchanged |
| PPP5D1 | 19-47030234-TG-CA | gCA/gTG | A/V | Unchanged |
| CCDC9 | 19-47774772-TG-CA | cTG/cCA | L/P | Unchanged |
| FAM83E | 19-49116357-GGT-AGC | AcC/GcT | T/A | Unchanged |
| SPHK2 | 19-49132360-TG-CA | cTG/cCA | L/P | Unchanged |
| SIGLEC11 | 19-50461664-TG-CA | tCA/tTG | S/L | Unchanged |
| VRK3 | 19-50491733-GCT-ACC | AgC/GgT | S/G | Unchanged |
| KLK14 | 19-51584951-TG-CA | CAg/TGg | Q/W | Rescued nonsense |
| SIGLEC9 | 19-51628481-TG-CA | TGg/CAg | W/Q | Rescued nonsense |
| SIGLEC9 | 19-51631236-TG-CA | gTG/gCA | V/A | Unchanged |
| SIGLEC12 | 19-52000672-GT-AC | ACg/GTg | T/V | Changed missense |
| ZNF577 | 19-52376119-GT-AC | ACt/GTt | T/V | Changed missense |
| ZNF615 | 19-52497745-TG-CA | CAg/TGg | Q/W | Rescued nonsense |
| ZNF28 | 19-53303745-TTG-CTA | CaA/TaG | Q/* | Unchanged |
| ZNF468 | 19-53344426-TG-CA | CAt/TGt | H/C | Changed missense |
| ZNF665 | 19-53669273-TG-CA | CAt/TGt | H/C | Changed missense |
| VN1R2 | 19-53761740-TG-CA | TGc/CAc | C/H | Changed missense |
| VN1R4 | 19-53770385-TG-CA | aCA/aTG | T/M | Changed missense |
| ZNF331 | 19-54080067-TG-CA | TGt/CAt | C/H | Changed missense |
| CNOT3 | 19-54652202-GT-AC | gGT/gAC | G/D | Unchanged |
| MBOAT7 | 19-54677759-TG-CA | cCA/cTG | P/L | Unchanged |
| MBOAT7 | 19-54677759-TGG-CGA | CcA/TcG | P/S | Unchanged |
| LILRB3 | 19-54725755-TG-CA | aCA/aTG | T/M | Changed missense |
| LILRB3 | 19-54726226-TG-CA | gCA/gTG | A/V | Unchanged |
| LILRA6 | 19-54745507-TG-CA | aCA/aTG | T/M | Changed missense |
| LILRB2 | 19-54784130-TG-CA | CAc/TGc | H/C | Changed missense |
| LILRA4 | 19-54844900-TG-CA | gCA/gTG | A/V | Unchanged |
| AC009892.10 | 19-55147504-TG-CA | gCA/gTG | A/V | Unchanged |
| KIR2DL1 | 19-55286650-TG-CA | cTG/cCA | L/P | Unchanged |
| TMEM86B | 19-55738634-TG-CA | CAg/TGg | H/C | Changed missense |
| SSC5D | 19-56024519-TG-CA | cTG/cCA | L/P | Unchanged |
| FIZ1 | 19-56104134-GGT-AGC | AcC/GcT | T/A | Unchanged |
| FIZ1 | 19-56104135-GT-AC | ACc/GTc | T/V | Changed missense |
| NLRP4 | 19-56369591-GCT-ACC | GcT/AcC | A/T | Unchanged |
| NLRP8 | 19-56473459-GT-AC | cGT/cAC | R/H | Unchanged |
| NLRP8 | 19-56477710-TG-CA | gTG/gCA | V/A | Unchanged |
| ZSCAN5C | 19-56717604-GGT-AGC | GgT/AgC | G/S | Unchanged |
| ZSCAN5D | 19-56751782-TG-CA | TGc/CAc | C/H | Changed missense |
| ZNF667 | 19-56953863-TG-CA | aCA/aTG | T/M | Changed missense |
| ZNF749 | 19-57956186-TG-CA | gTG/gCA | V/A | Unchanged |
| A1BG | 19-58864479-TG-CA | CAc/TGc | H/C | Changed missense |
| TPO | 2-1520676-TG-CA | gTG/gCA | V/A | Unchanged |
| APOB | 2-21232804-GT-AC | gAC/gGT | D/G | Unchanged |
| ADCY3 | 2-25046090-TG-CA | tCA/tTG | S/L | Unchanged |
| KIF3C | 2-26203678-TG-CA | CAg/TGg | Q/W | Rescued nonsense |
| DRC1 | 2-26667615-TG-CA | TGg/CAg | W/Q | Rescued nonsense |
| CIB4 | 2-26804247-TG-CA | CAc/TGc | H/C | Changed missense |
| SLC5A6 | 2-27423929-TG-CA | cCA/cTG | P/L | Unchanged |
| GCKR | 2-27730940-TG-CA | cTG/cCA | L/P | Unchanged |
| AC074091.13 | 2-27930502-GT-CA | aAC/aTG | N/M | Changed missense |
| PLB1 | 2-28865760-TTG-CTA | TtG/CtA | L | Unchanged |
| FAM179A | 2-29246044-TG-CA | gTG/gCA | V/A | Unchanged |
| LTBP1 | 2-33585795-GT-AC | GTg/ACg | V/T | Changed missense |
| LTBP1 | 2-33585796-TG-CA | gTG/gCA | V/A | Unchanged |
| FEZ2 | 2-36810586-TG-CA | aCA/aTG | T/M | Changed missense |
| HAAO | 2-43015717-GAT-AAC | AtC/GtT | I/V | Unchanged |
| ABCG8 | 2-44104924-GT-AC | GTc/ACc | V/T | Changed missense |
| EPCAM | 2-47601106-TG-CA | aTG/aCA | M/T | Partially changed missense |
| ETAA1 | 2-67630476-TG-CA | aTG/aCA | M/T | Partially changed missense |
| ARHGAP25 | 2-69049941-TG-CA | aTG/aCA | M/T | Partially changed missense |
| ASPRV1 | 2-70188675-GT-AC | ACt/GTt | T/V | Changed missense |
| REG3G | 2-79253947-TG-CA | aTG/aCA | M/T | Partially changed missense |
| REG3A | 2-79385786-TG-CA | aCA/aTG | T/M | Changed missense |
| CTNNA2 | 2-80801344-GTT-ATC | GtT/AtC | V/I | Unchanged |
| IMMT | 2-86371883-TG-CA | gCA/gTG | A/V | Unchanged |
| IGKJ3 | 2-89160737-GAT-AAC | AtC/GtT | I/V | Unchanged |
| IGKJ2 | 2-89161053-GGT-AGC | AcC/GcT | T/A | Unchanged |
| IGKJ2 | 2-89161054-GT-AC | ACc/GTc | T/V | Changed missense |
| IGKV1-5 | 2-89246850-GT-AC | ACt/GTt | T/V | Changed missense |
| IGKV1-5 | 2-89247011-GCT-ACC | AgC/GgT | S/G | Unchanged |
| IGKV3-7 | 2-89278101-GCT-ACC | AgC/GgT | S/G | Unchanged |
| IGKV1-8 | 2-89292169-GT-AC | ACa/GTa | T/V | Changed missense |
| IGKV3-11 | 2-89326863-GCT-ACC | AgC/GgT | S/G | Unchanged |
| IGKV3-15 | 2-89384784-GAT-AAC | AtC/GtT | I/V | Unchanged |
| IGKV3-15 | 2-89384814-GAT-CAA | AtC/TtG | I/L | Changed missense |
| IGKV1-33 | 2-89567875-TG-AC | aCA/aGT | T/S | Changed missense |
| IGKV2D-28 | 2-89999461-GT-AC | aGT/aAC | S/N | Unchanged |
| ASTL | 2-96795857-TG-CA | CAg/TGg | Q/W | Rescued nonsense |
| FAM178B | 2-97613616-TG-CA | aCA/aTG | T/M | Changed missense |
| TBC1D8 | 2-101656725-GT-AC | ACg/GTg | T/V | Changed missense |
| IL1RL1 | 2-102968362-TG-CA | tTG/tCA | L/S | Unchanged |
| MYO7B | 2-128338393-TG-CA | cTG/cCA | L/P | Unchanged |
| POTEF | 2-130832358-TG-CA | CAt/TGt | H/C | Changed missense |
| CCDC74B | 2-130899804-TG-CA | CAt/TGt | H/C | Changed missense |
| SMPD4 | 2-130910452-TG-CA | gCA/gTG | A/V | Unchanged |
| TUBA3E | 2-130951584-TG-CA | tCA/tTG | S/L | Unchanged |
| POTEI | 2-131221283-GT-AC | aAC/aGT | N/S | Unchanged |
| POTEI | 2-131221445-GT-AC | gAC/gGT | D/G | Unchanged |
| POTEJ | 2-131414431-TG-CA | TGc/CAc | C/H | Changed missense |
| NCKAP5 | 2-133541444-TGG-CGA | CcA/TcG | P/S | Unchanged |
| LRP1B | 2-141459829-TG-CA | aCA/aTG | T/M | Changed missense |
| LRP1B | 2-141571329-TG-CA | tCA/tTG | S/L | Unchanged |
| LRP1B | 2-142567910-TG-CA | CAg/TGg | Q/W | Rescued nonsense |
| NEB | 2-152387553-TG-CA | aCA/aTG | T/M | Changed missense |
| CCDC148 | 2-159201754-TG-CA | CAg/TGg | Q/W | Rescued nonsense |
| DAPL1 | 2-159663599-TG-CA | cTG/cCA | L/P | Unchanged |
| LY75 | 2-160639970-TG-CA | tCA/tTG | S/L | Unchanged |
| CD302 | 2-160639970-TG-CA | tCA/tTG | S/L | Unchanged |
| LY75-CD302 | 2-160639970-TG-CA | tCA/tTG | S/L | Unchanged |
| LY75-CD302 | 2-160710922-TG-CA | gCA/gTG | A/V | Unchanged |
| LY75 | 2-160710922-TG-CA | gCA/gTG | A/V | Unchanged |
| LY75 | 2-160711045-GT-CA | gAC/gTG | D/V | Partially changed missense |
| LY75-CD302 | 2-160711045-GT-CA | gAC/gTG | D/V | Partially changed missense |
| SCN1A | 2-166848634-TAG-CAA | CtA/TtG | L | Unchanged |
| ABCB11 | 2-169789016-TG-CA | gCA/gTG | A/V | Unchanged |
| LRP2 | 2-169997051-GAT-AAC | AtC/GtT | I/V | Unchanged |
| LRP2 | 2-170103336-TG-CA | aCA/aTG | T/M | Changed missense |
| TTN | 2-179428672-TG-AC | CAa/GTa | Q/V | Changed missense |
| TTN | 2-179430997-GT-AC | ACg/GTg | T/V | Changed missense |
| TTN | 2-179517654-TG-CA | cCA/cTG | P/L | Unchanged |
| FSIP2 | 2-186656956-TG-CA | aTG/aCA | M/T | Partially changed missense |
| COL5A2 | 2-189932831-TG-CA | cCA/cTG | P/L | Unchanged |
| COL5A2 | 2-189932831-TGG-CGA | CcA/TcG | P/S | Unchanged |
| SLC40A1 | 2-190430141-TAG-CAA | CtA/TtG | L | Unchanged |
| MYO1B | 2-192279327-TTG-CTA | TtG/CtA | L | Unchanged |
| PLCL1 | 2-198950240-GT-AC | GTa/ACa | V/T | Changed missense |
| TRAK2 | 2-202260049-GT-AC | cAC/cGT | H/R | Unchanged |
| RAPH1 | 2-204305093-TG-CA | cCA/cTG | P/L | Unchanged |
| PIKFYVE | 2-209190330-TG-CA | tTG/tCA | L/S | Unchanged |
| ABCA12 | 2-215865575-TG-CA | cCA/cTG | P/L | Unchanged |
| TNS1 | 2-218750817-TG-CA | cCA/cTG | P/L | Unchanged |
| ASIC4 | 2-220402680-TG-CA | gTG/gCA | V/A | Unchanged |
| CHPF | 2-220405321-TG-CA | CAg/TGg | Q/W | Rescued nonsense |
| OBSL1 | 2-220419236-TG-CA | aCA/aTG | T/M | Changed missense |
| IRS1 | 2-227661043-TG-CA | gCA/gTG | A/V | Unchanged |
| COL4A3 | 2-228111435-TG-CA | cTG/cCA | L/P | Unchanged |
| TRIP12 | 2-230668858-TG-CA | aCA/aTG | T/M | Changed missense |
| SP140 | 2-231175549-TG-CA | TGt/CAt | C/H | Changed missense |
| SP100 | 2-231406680-TG-CA | aTG/aCA | M/T | Partially changed missense |
| PSMD1 | 2-231934755-TG-CA | aTG/aCA | M/T | Partially changed missense |
| ECEL1 | 2-233349587-TG-CA | CAt/TGt | H/C | Changed missense |
| KCNJ13 | 2-233633459-TG-CA | aCA/aTG | T/M | Changed missense |
| UGT1A5 | 2-234621780-TG-CA | tTG/tCA | L/S | Unchanged |
| UGT1A5 | 2-234621810-TG-CA | gTG/gCA | V/A | Unchanged |
| UGT1A3 | 2-234637803-TG-CA | TGg/CAg | W/Q | Rescued nonsense |
| UGT1A8 | 2-234637803-TG-CA | TGg/CAg | W/Q | Rescued nonsense |
| MROH2A | 2-234725502-TG-CA | TGg/CAg | W/Q | Rescued nonsense |
| SH3BP4 | 2-235950185-GAT-AAC | GaT/AaC | D/N | Unchanged |
| COL6A3 | 2-238243285-GT-AC | cAC/cGT | H/R | Unchanged |
| ESPNL | 2-239039637-TG-CA | gTG/gCA | V/A | Unchanged |
| ESPNL | 2-239040185-GGT-AGC | GgT/AgC | G/S | Unchanged |
| NDUFA10 | 2-240944638-GT-AC | cAC/cGT | H/R | Unchanged |
| OR6B3 | 2-240985347-GT-AC | ACc/GTc | T/V | Changed missense |
| GPR35 | 2-241569704-TG-AC | gTG/gAC | V/D | Changed missense |
| PASK | 2-242066314-TG-CA | gCA/gTG | A/V | Unchanged |
| PASK | 2-242066374-GT-AC | aAC/aGT | N/S | Unchanged |
| PASK | 2-242082262-TG-CA | aCA/aTG | T/M | Changed missense |
| DEFB132 | 20-239936-GT-AC | GTa/ACa | V/T | Changed missense |
| SIRPG | 20-1610894-GT-AC | tAC/tGT | Y/C | Unchanged |
| SIRPA | 20-1895796-TG-CA | tTG/tCA | L/S | Unchanged |
| SIRPA | 20-1896059-GT-AC | GTg/ACg | V/T | Changed missense |
| TGM6 | 20-2375106-GT-AC | GTc/ACc | V/T | Changed missense |
| ZNF343 | 20-2464059-TG-CA | tCA/tTG | S/L | Unchanged |
| NOP56 | 20-2638579-TG-CA | aTG/aCA | M/T | Partially changed missense |
| C20orf196 | 20-5843810-GGT-AGC | GgT/AgC | G/S | Unchanged |
| PLCB1 | 20-8755241-GCT-ACC | GcT/AcC | A/T | Unchanged |
| BFSP1 | 20-17474968-TG-CA | cCA/cTG | P/L | Unchanged |
| DZANK1 | 20-18445919-GTT-ATC | AaC/GaT | N/D | Unchanged |
| CSTL1 | 20-23424613-TG-CA | TGg/CAg | W/Q | Rescued nonsense |
| CST3 | 20-23618395-TAG-CAA | CtA/TtG | L | Unchanged |
| ACSS1 | 20-25011423-TG-CA | gCA/gTG | A/V | Unchanged |
| PYGB | 20-25264812-GAT-AAC | GaT/AaC | D/N | Unchanged |
| ASXL1 | 20-31022959-TG-CA | cTG/cCA | L/P | Unchanged |
| BPIFB4 | 20-31672757-GT-AC | gGT/gAC | G/D | Unchanged |
| CPNE1 | 20-34219496-TG-CA | CAa/TGa | Q/* | Unchanged |
| KIAA1755 | 20-36841756-TG-CA | tCA/tTG | S/L | Unchanged |
| SEMG2 | 20-43851094-GT-AC | aGT/aAC | S/N | Unchanged |
| PREX1 | 20-47249170-GT-AC | aAC/aGT | N/S | Unchanged |
| TSHZ2 | 20-51870679-GT-AC | GTc/ACc | V/T | Changed missense |
| TSHZ2 | 20-51871883-GT-AC | aGT/aAC | S/N | Unchanged |
| PCK1 | 20-56139402-GT-AC | gGT/gAC | G/D | Unchanged |
| ZBP1 | 20-56186884-TG-CA | CAg/TGg | Q/W | Rescued nonsense |
| LAMA5 | 20-60912683-TG-CA | aCA/aTG | T/M | Changed missense |
| LAMA5 | 20-60921641-GGT-AGC | AcC/GcT | T/A | Unchanged |
| LAMA5 | 20-60926828-GAT-AAC | AtC/GtT | I/V | Unchanged |
| GATA5 | 20-61039958-TG-CA | cCA/cTG | P/L | Unchanged |
| C20orf166 | 20-61162266-GT-AC | GTg/ACg | V/T | Changed missense |
| C20orf166 | 20-61162267-TG-CA | gTG/gCA | V/A | Unchanged |
| C20orf166 | 20-61167882-TG-CA | TGa/CAa | */Q | Unchanged |
| DIDO1 | 20-61528074-TG-CA | gCA/gTG | A/V | Unchanged |
| HELZ2 | 20-62193445-GT-AC | ACg/GTg | T/V | Changed missense |
| HELZ2 | 20-62196806-GT-AC | cAC/cGT | H/R | Unchanged |
| HELZ2 | 20-62196807-TG-CA | CAc/TGc | H/C | Changed missense |
| HELZ2 | 20-62200575-TG-CA | tCA/tTG | S/L | Unchanged |
| GMEB2 | 20-62221610-TAG-CAA | CtA/TtG | L | Unchanged |
| GMEB2 | 20-62236148-TG-CA | gCA/gTG | A/V | Unchanged |
| TNFRSF6B | 20-62328452-GT-AC | cGT/cAC | R/H | Unchanged |
| SAMD10 | 20-62608654-TG-AC | CAt/GTt | H/V | Changed missense |
| NPBWR2 | 20-62737568-TG-CA | CAg/TGg | Q/W | Rescued nonsense |
| SAMSN1 | 21-15954527-TG-CA | CAc/TGc | H/C | Changed missense |
| NCAM2 | 21-22746187-TG-CA | cTG/cCA | L/P | Unchanged |
| LTN1 | 21-30339232-GCT-ACC | AgC/GgT | S/G | Unchanged |
| SCAF4 | 21-33068962-TG-CA | gCA/gTG | A/V | Unchanged |
| ITSN1 | 21-35260480-GT-AC | cGT/cAC | R/H | Unchanged |
| FKSG68 | 21-37270507-TG-CA | CAg/TGg | Q/W | Rescued nonsense |
| TTC3 | 21-38525356-TG-CA | aTG/aCA | M/T | Partially changed missense |
| KCNJ6 | 21-39086965-TG-CA | cCA/cTG | P/L | Unchanged |
| ERG | 21-39775468-GT-AC | aAC/aGT | N/S | Unchanged |
| PSMG1 | 21-40549466-TG-CA | cCA/cTG | P/L | Unchanged |
| B3GALT5 | 21-41032740-TG-CA | aTG/aCA | M/T | Partially changed missense |
| MX1 | 21-42821112-GT-AC | cGT/cAC | R/H | Unchanged |
| TMPRSS2 | 21-42866296-TG-CA | aCA/aTG | T/M | Changed missense |
| UMODL1 | 21-43531008-TG-CA | aTG/aCA | M/T | Partially changed missense |
| RRP1B | 21-45107562-TG-CA | cTG/cCA | L/P | Unchanged |
| RRP1 | 21-45219522-GTT-ATC | GtT/AtC | V/I | Unchanged |
| C21orf33 | 21-45553595-GT-AC | GTc/ACc | V/T | Changed missense |
| AIRE | 21-45717548-GAT-AAC | GaT/AaC | D/N | Unchanged |
| KRTAP10-1 | 21-45959854-TG-CA | gCA/gTG | A/V | Unchanged |
| KRTAP10-2 | 21-45971225-TG-CA | cCA/cTG | P/L | Unchanged |
| KRTAP10-3 | 21-45977994-GT-AC | ACg/GTg | T/V | Changed missense |
| KRTAP10-3 | 21-45978194-TG-CA | tCA/tTG | S/L | Unchanged |
| KRTAP10-3 | 21-45978338-TG-CA | cCA/cTG | P/L | Unchanged |
| KRTAP10-6 | 21-46011718-TG-CA | cCA/cTG | P/L | Unchanged |
| KRTAP10-6 | 21-46011718-TGG-CGA | CcA/TcG | P/S | Unchanged |
| KRTAP10-6 | 21-46012154-GT-AC | ACc/GTc | T/V | Changed missense |
| KRTAP10-7 | 21-46020523-TG-CA | aTG/aCA | M/T | Unchanged |
| KRTAP10-7 | 21-46021390-GT-CA | aGT/aCA | S/T | Partially changed missense |
| KRTAP10-10 | 21-46058034-GT-AC | GTg/ACg | V/T | Changed missense |
| KRTAP10-10 | 21-46058035-TG-CA | gTG/gCA | V/A | Unchanged |
| COL6A1 | 21-47410929-GGT-AGC | GgT/AgC | G/S | Unchanged |
| COL6A2 | 21-47538960-GT-AC | aGT/aAC | S/N | Unchanged |
| AP001468.1 | 21-47612552-TG-CA | CAt/TGt | H/C | Changed missense |
| AP001468.1 | 21-47613004-TG-CA | cCA/cTG | P/L | Unchanged |
| PCNT | 21-47787001-GT-AC | GTg/ACg | V/T | Changed missense |
| PCNT | 21-47787002-TG-CA | gTG/gCA | V/A | Unchanged |
| CECR1 | 22-17669306-TG-CA | CAt/TGt | H/C | Changed missense |
| AC008132.13 | 22-18835523-TG-CA | gTG/gCA | V/A | Unchanged |
| CLTCL1 | 22-19197896-TG-CA | gCA/gTG | A/V | Unchanged |
| FAM230A | 22-20709734-GT-AC | gGT/gAC | G/D | Unchanged |
| POM121L7 | 22-21481404-TG-CA | gCA/gTG | A/V | Unchanged |
| GGT2 | 22-21563319-TG-CA | gCA/gTG | A/V | Unchanged |
| PPM1F | 22-22287862-GT-AC | aAC/aGT | N/S | Unchanged |
| IGLV7-46 | 22-22724394-TG-CA | tTG/tCA | L/S | Unchanged |
| IGLV1-40 | 22-22764409-GGT-CGA | GgT/CgA | G/R | Unchanged |
| IGLV2-23 | 22-23040770-GT-CA | aGT/aCA | S/T | Partially changed missense |
| IGLV2-11 | 22-23135287-GT-CA | gGT/gCA | G/A | Unchanged |
| IGLV3-1 | 22-23223435-GAT-AAC | GaT/AaC | D/N | Unchanged |
| ZNF70 | 22-24085990-TAG-CAA | CtA/TtG | L | Unchanged |
| CABIN1 | 22-24459437-GT-AC | tGT/tAC | C/Y | Unchanged |
| MYO18B | 22-26173661-TG-CA | TGg/CAg | W/Q | Rescued nonsense |
| MYO18B | 22-26194043-GGT-AGC | GgT/AgC | G/S | Unchanged |
| MYO18B | 22-26194044-GT-AC | gGT/gAC | G/D | Unchanged |
| C22orf31 | 22-29456733-TG-CA | tCA/tTG | S/L | Unchanged |
| SEC14L6 | 22-30934816-TG-CA | CAa/TGa | Q/* | Unchanged |
| OSBP2 | 22-31266544-GCT-ACC | GcT/AcC | A/T | Unchanged |
| MORC2 | 22-31333631-TG-CA | cCA/cTG | P/L | Unchanged |
| RFPL3 | 22-32756682-GT-AC | GTc/ACc | V/T | Changed missense |
| APOL1 | 22-36661565-TG-CA | aTG/aCA | M/T | Partially changed missense |
| FOXRED2 | 22-36897427-TG-CA | gCA/gTG | A/V | Unchanged |
| C22orf23 | 22-38341134-TG-CA | aCA/aTG | T/M | Changed missense |
| APOBEC3F | 22-39445536-GT-AC | GTa/ACa | V/T | Changed missense |
| APOBEC3F | 22-39448283-TG-CA | TGg/CAg | W/Q | Rescued nonsense |
| SERHL2 | 22-42970032-TG-CA | TGc/CAc | C/H | Changed missense |
| A4GALT | 22-43089539-TG-CA | CAg/TGg | Q/W | Rescued nonsense |
| ARFGAP3 | 22-43195109-TG-CA | CAg/TGg | Q/W | Rescued nonsense |
| TTLL12 | 22-43576904-GT-AC | cAC/cGT | H/R | Unchanged |
| PARVB | 22-44395451-TG-CA | TGg/CAg | W/Q | Rescued nonsense |
| PARVB | 22-44489868-TG-CA | gTG/gCA | V/A | Unchanged |
| PRR5 | 22-45075738-TG-CA | aTG/aCA | M/T | Partially changed missense |
| PRR5- | 22-45128231-GT-AC | cGT/cAC | R/H | Unchanged |
| ARHGAP8 | | | | |
| PRR5 | 22-45128231-GT-AC | cGT/cAC | R/H | Unchanged |
| PRR5- | 22-45255629-GT-AC | cGT/cAC | R/H | Unchanged |
| ARHGAP8 | | | | |
| ARHGAP8 | 22-45255629-GT-AC | cGT/cAC | R/H | Unchanged |
| C22orf26 | 22-46447714-TG-CA | CAg/TGg | Q/W | Rescued nonsense |
| FLJ27365 | 22-46505786-TG-CA | TGg/CAg | W/Q | Rescued nonsense |
| GTSE1 | 22-46722400-TG-CA | TGg/CAg | W/Q | Rescued nonsense |
| CTA-299D3.8 | 22-48940551-GCT-ACC | AgC/GgT | S/G | Unchanged |
| ALG12 | 22-50301476-TG-CA | gCA/gTG | A/V | Unchanged |
| PIM3 | 22-50356692-GT-AC | GTc/ACc | V/T | Changed missense |
| MLC1 | 22-50515270-TG-CA | tCA/tTG | S/L | Unchanged |
| DENND6B | 22-50754648-TG-CA | gCA/gTG | A/V | Unchanged |
| LMF2 | 22-50941985-TTG-CTA | CaA/TaG | Q/* | Unchanged |
| LMF2 | 22-50944128-GT-AC | cAC/cGT | H/R | Unchanged |
| ATG7 | 3-11596300-GAT-AAC | GaT/AaC | D/N | Unchanged |
| CAND2 | 3-12861608-GT-AC | GTc/ACc | V/T | Changed missense |
| TMEM43 | 3-14175262-TG-CA | aTG/aCA | M/T | Partially changed missense |
| RFTN1 | 3-16358584-TG-CA | tCA/tTG | S/L | Unchanged |
| EFHB | 3-19959756-GT-AC | ACc/GTc | T/V | Changed missense |
| DYNC1LI1 | 3-32611851-GTT-ATC | AaC/GaT | N/D | Unchanged |
| TRANK1 | 3-36898715-GT-AC | ACc/GTc | T/V | Changed missense |
| PLCD1 | 3-38051941-TG-CA | cCA/cTG | P/L | Unchanged |
| SLC22A14 | 3-38350519-GT-AC | GTg/ACg | V/T | Changed missense |
| XYLB | 3-38401838-GT-AC | gGT/gAC | G/D | Unchanged |
| SCN5A | 3-38674712-TG-CA | gCA/gTG | A/V | Unchanged |
| XIRP1 | 3-39229900-TG-CA | CAg/TGg | Q/W | Rescued nonsense |
| KLHL40 | 3-42733468-TG-CA | TGc/CAc | C/H | Changed missense |
| TOPAZ1 | 3-44283672-TG-CA | TGc/CAc | C/H | Changed missense |
| CDCP1 | 3-45134822-TG-CA | CAa/TGa | Q/* | Unchanged |
| CCDC12 | 3-47018214-TG-CA | gCA/gTG | A/V | Unchanged |
| COL7A1 | 3-48625266-TG-CA | cCA/cTG | P/L | Unchanged |
| CCDC71 | 3-49200692-TG-AC | CAg/GTg | Q/V | Changed missense |
| BSN | 3-49693411-TG-CA | cTG/cCA | L/P | Unchanged |
| MST1 | 3-49724808-TG-CA | cCA/cTG | P/L | Unchanged |
| GMPPB | 3-49760039-TG-CA | CAg/TGg | Q/W | Rescued nonsense |
| CAMKV | 3-49897244-TG-AC | CAg/GTg | Q/V | Changed missense |
| PCBP4 | 3-51994587-TG-CA | gCA/gTG | A/V | Unchanged |
| RP11- | 3-51994587-TG-CA | gCA/gTG | A/V | Unchanged |
| 155D18.14 | | | | |
| STAB1 | 3-52555443-GT-CA | aGT/aCA | S/T | Partially changed missense |
| STAB1 | 3-52555956-GT-AC | cGT/cAC | R/H | Unchanged |
| RFT1 | 3-53125922-TG-CA | aCA/aTG | T/M | Changed missense |
| PRKCD | 3-53213655-GAT-AAC | GaT/AaC | D/N | Unchanged |
| FLNB | 3-58118555-GT-AC | GTg/ACg | V/T | Changed missense |
| KBTBD8 | 3-67054649-TG-CA | TGt/CAt | C/H | Changed missense |
| FRMD4B | 3-69245453-GT-AC | ACg/GTg | T/V | Changed missense |
| ZNF717 | 3-75787298-TG-CA | tCA/tTG | S/L | Unchanged |
| EPHA3 | 3-89521693-TG-CA | TGg/CAg | W/Q | Rescued nonsense |
| OR5AC2 | 3-97806615-TG-CA | aTG/aCA | M/T | Partially changed missense |
| OR5H6 | 3-97983711-TG-CA | TGt/CAt | C/H | Changed missense |
| OR5K1 | 3-98189157-TG-CA | tTG/tCA | L/S | Unchanged |
| IMPG2 | 3-100963154-GT-AC | ACa/GTa | T/V | Changed missense |
| ALCAM | 3-105260594-GCT-ACC | GcT/AcC | A/T | Unchanged |
| CBLB | 3-105400420-TG-CA | cCA/cTG | P/L | Unchanged |
| MORC1 | 3-108690220-TG-CA | CAt/TGt | H/C | Changed missense |
| DPPA4 | 3-109049384-TG-CA | cCA/cTG | P/L | Unchanged |
| POPDC2 | 3-119367390-TG-CA | tCA/tTG | S/L | Unchanged |
| FBXO40 | 3-121340536-TG-CA | gTG/gCA | V/A | Unchanged |
| GOLGB1 | 3-121412618-TG-CA | CAt/TGt | H/C | Changed missense |
| GOLGB1 | 3-121435621-TTG-CTA | CaA/TaG | Q/* | Unchanged |
| CD86 | 3-121825197-GTT-ATC | GtT/AtC | V/I | Unchanged |
| SEMA5B | 3-122634716-GT-AC | gAC/gGT | D/G | Unchanged |
| MYLK | 3-123452838-GGT-AGC | AcC/GcT | T/A | Unchanged |
| ALDH1L1 | 3-125826058-GT-AC | gAC/gGT | D/G | Unchanged |
| EFCAB12 | 3-129140489-TG-CA | aCA/aTG | T/M | Changed missense |
| PLXND1 | 3-129291751-TG-CA | cCA/cTG | P/L | Unchanged |
| PLXND1 | 3-129293182-GT-AC | cAC/cGT | H/R | Unchanged |
| PLXND1 | 3-129293183-TG-CA | CAc/TGc | H/C | Changed missense |
| TMCC1 | 3-129546727-GCT-ACC | AgC/GgT | S/G | Unchanged |
| SLCO2A1 | 3-133670073-TCG-CCA | CgA/TgG | R/W | Rescued nonsense |
| DZIP1L | 3-137796377-GGT-AGC | AcC/GcT | T/A | Unchanged |
| FAIM | 3-138347988-TG-CA | tTG/tCA | L/S | Unchanged |
| HLTF | 3-148759325-TG-CA | gCA/gTG | A/V | Unchanged |
| P2RY13 | 3-151046308-GT-AC | ACg/GTg | T/V | Changed missense |
| IGSF10 | 3-151154666-TG-CA | aCA/aTG | T/M | Changed missense |
| MME | 3-154861350-GT-AC | aGT/aAC | S/N | Unchanged |
| GFM1 | 3-158366900-GT-AC | GTa/ACa | V/T | Changed missense |
| LXN | 3-158387350-TG-CA | CAa/TGa | Q/* | Unchanged |
| RARRES1 | 3-158450079-GT-AC | gAC/gGT | D/G | Unchanged |
| C3orf80 | 3-159943645-GT-CA | GTg/CAg | V/Q | Changed missense |
| SI | 3-164767022-GT-CA | gAC/gTG | D/V | Partially changed missense |
| NCEH1 | 3-172428782-TG-CA | CAg/TGg | Q/W | Rescued nonsense |
| NAALADL2 | 3-174951756-TG-CA | aTG/aCA | M/T | Partially changed missense |
| AHSG | 3-186337713-TG-CA | aTG/aCA | M/T | Partially changed missense |
| CPN2 | 3-194061906-TG-CA | CAg/TGg | Q/W | Rescued nonsense |
| LRRC15 | 3-194080648-GT-AC | aAC/aGT | N/S | Unchanged |
| LRRC15 | 3-194081152-GTT-ATC | AaC/GaT | N/D | Unchanged |
| LRRC15 | 3-194081635-TG-CA | gCA/gTG | A/V | Unchanged |
| MUC4 | 3-195505662-TGG-CGA | CcA/TcG | P/S | Unchanged |
| MUC4 | 3-195505848-TG-CA | gCA/gTG | A/V | Unchanged |
| MUC4 | 3-195507081-GGT-AGC | AcC/GcT | T/A | Unchanged |
| MUC4 | 3-195507096-TG-CA | gCA/gTG | A/V | Unchanged |
| MUC4 | 3-195507322-GT-AC | ACc/GTc | T/V | Changed missense |
| MUC4 | 3-195509496-TG-CA | gCA/gTG | A/V | Unchanged |
| MUC4 | 3-195510168-TG-CA | gCA/gTG | A/V | Unchanged |
| MUC4 | 3-195510201-GGT-AGC | AcC/GcT | T/A | Unchanged |
| MUC4 | 3-195511512-TG-CA | gCA/gTG | A/V | Unchanged |
| MUC4 | 3-195511560-TG-CA | gCA/gTG | A/V | Unchanged |
| MUC4 | 3-195512187-TG-CA | tCA/tTG | S/L | Unchanged |
| MUC4 | 3-195512328-TG-CA | gCA/gTG | A/V | Unchanged |
| MUC4 | 3-195515385-GGT-AGC | AcC/GcT | T/A | Unchanged |
| MUC4 | 3-195515386-GT-AC | ACc/GTc | T/V | Changed missense |
| MUC4 | 3-195515482-GT-AC | ACc/GTc | T/V | Changed missense |
| WDR53 | 3-196281208-TG-CA | cCA/cTG | P/L | Unchanged |
| PIGZ | 3-196674973-TG-CA | gCA/gTG | A/V | Unchanged |
| MFI2 | 3-196742290-TG-CA | cCA/cTG | P/L | Unchanged |
| MFI2 | 3-196743130-TG-CA | aCA/aTG | T/M | Changed missense |
| ZNF721 | 4-436379-GT-AC | ACg/GTg | T/V | Changed missense |
| GAK | 4-858941-TG-CA | gCA/gTG | A/V | Unchanged |
| TMEM175 | 4-951947-TG-CA | aTG/aCA | M/T | Partially changed missense |
| SLC26A1 | 4-983060-TG-CA | CAg/TGg | Q/W | Rescued nonsense |
| FGFRL1 | 4-1018269-GAT-AAC | GaT/AaC | D/N | Unchanged |
| FGFRL1 | 4-1018749-TG-CA | TGg/CAg | W/Q | Rescued nonsense |
| CRIPAK | 4-1388363-GT-AC | GTg/ACg | V/T | Changed missense |
| CRIPAK | 4-1388411-TG-CA | TGt/CAt | C/H | Changed missense |
| CRIPAK | 4-1388412-GT-AC | tGT/tAC | C/Y | Unchanged |
| CRIPAK | 4-1389156-TG-CA | aTG/aCA | M/T | Partially changed missense |
| CRIPAK | 4-1389366-GT-AC | cGT/cAC | R/H | Unchanged |
| FAM53A | 4-1657281-GGT-AGC | AcC/GcT | T/A | Unchanged |
| TMEM129 | 4-1719294-TG-CA | aCA/aTG | T/M | Changed missense |
| POLN | 4-2181080-TG-CA | aCA/aTG | T/M | Changed missense |
| HAUS3 | 4-2242083-TTG-ATC | CaA/GaT | Q/D | Changed missense |
| ZFYVE28 | 4-2341194-TG-CA | gCA/gTG | A/V | Unchanged |
| GRK4 | 4-3037213-TG-CA | cTG/cCA | L/P | Unchanged |
| GRK4 | 4-3039150-TG-CA | gTG/gCA | V/A | Unchanged |
| HTT | 4-3174738-GT-AC | aGT/aAC | S/N | Unchanged |
| LYAR | 4-4276131-GT-AC | cAC/cGT | H/R | Unchanged |
| LYAR | 4-4276132-TG-CA | CAc/TGc | H/C | Changed missense |
| EVC2 | 4-5624669-GT-AC | ACg/GTg | T/V | Changed missense |
| C4orf50 | 4-5991382-GTT-ATC | AaC/GaT | N/D | Unchanged |
| PSAPL1 | 4-7436151-TG-CA | cCA/cTG | P/L | Unchanged |
| SORCS2 | 4-7714488-GAT-AAC | GaT/AaC | D/N | Unchanged |
| USP17L10 | 4-9213568-GT-AC | GTa/ACa | V/T | Changed missense |
| PPARGC1A | 4-23815924-TG-CA | aCA/aTG | T/M | Changed missense |
| RFC1 | 4-39302029-TG-CA | cCA/cTG | P/L | Unchanged |
| UGDH | 4-39512347-TG-CA | cCA/cTG | P/L | Unchanged |
| KCTD8 | 4-44176960-TG-CA | aCA/aTG | T/M | Changed missense |
| COMMD8 | 4-47455085-TG-CA | gCA/gTG | A/V | Unchanged |
| ATP10D | 4-47525054-TG-CA | TGc/CAc | C/H | Changed missense |
| NFXL1 | 4-47887536-TG-CA | gCA/gTG | A/V | Unchanged |
| NFXL1 | 4-47887991-GGT-AGC | AcC/GcT | T/A | Unchanged |
| FRYL | 4-48582909-GCT-ACC | AgC/GgT | S/G | Unchanged |
| CLOCK | 4-56314977-GT-AC | ACa/GTa | T/V | Changed missense |
| NOA1 | 4-57829649-GT-AC | aAC/aGT | N/S | Unchanged |
| TMPRSS11A | 4-68784774-TG-CA | CAg/TGg | Q/W | Rescued nonsense |
| UGT2B17 | 4-69416556-TG-CA | gCA/gTG | A/V | Unchanged |
| UGT2B28 | 4-70146310-GT-CA | gGT/gCA | G/A | Unchanged |
| UGT2B4 | 4-70346496-GT-AC | cAC/cGT | H/R | Unchanged |
| UGT2B4 | 4-70355211-TG-CA | aCA/aTG | T/M | Changed missense |
| SMR3A | 4-71232430-TG-CA | TGt/CAt | C/H | Changed missense |
| GC | 4-72618296-TG-CA | CAc/TGc | H/C | Changed missense |
| FRAS1 | 4-79350318-TG-CA | cTG/cCA | L/P | Unchanged |
| GK2 | 4-80329343-TG-CA | cCA/cTG | P/L | Unchanged |
| HSD17B11 | 4-88258483-TG-CA | CAa/TGa | Q/* | Unchanged |
| DSPP | 4-88536419-GAT-AAC | GaT/AaC | D/N | Unchanged |
| DSPP | 4-88536549-GT-AC | aGT/aAC | S/N | Unchanged |
| DSPP | 4-88537403-GAT-AAC | GaT/AaC | D/N | Unchanged |
| HERC6 | 4-89311962-TG-CA | TGt/CAt | C/H | Changed missense |
| SMARCAD1 | 4-95170839-GT-AC | aGT/aAC | S/N | Unchanged |
| TBCK | 4-107168430-TG-AC | CAa/GTa | Q/V | Changed missense |
| ALPK1 | 4-113352898-TG-CA | aTG/aCA | M/T | Partially changed missense |
| ALPK1 | 4-113353285-TG-CA | aTG/aGA | M/T | Partially changed missense |
| NUDT6 | 4-123843716-TG-CA | cCA/cTG | P/L | Unchanged |
| NUDT6 | 4-123843716-TGG-CGA | CcA/TcG | P/S | Unchanged |
| ANKRD50 | 4-125590478-TG-CA | cCA/cTG | P/L | Unchanged |
| HSPA4L | 4-128723042-TG-CA | tTG/tCA | L/S | Unchanged |
| LARP1B | 4-129100643-GT-AC | cGT/cAC | R/H | Unchanged |
| PCDH18 | 4-138442525-GGT-AGC | AcC/GcT | T/A | Unchanged |
| MAML3 | 4-140640621-GT-CA | gAC/gTG | D/V | Partially changed missense |
| KIAA0922 | 4-154479429-GT-AC | cGT/cAC | R/H | Unchanged |
| PLRG1 | 4-155461113-TTG-CTA | CaA/TaG | Q/* | Unchanged |
| PALLD | 4-169433326-TG-CA | aTG/aCA | M/T | Partially changed missense |
| TENM3 | 4-183652173-GAT-AAC | GaT/AaC | D/N | Unchanged |
| ENPP6 | 4-185012396-GCT-ACC | AgC/GgT | S/G | Unchanged |
| CCDC110 | 4-186379371-GT-AC | tAC/tGT | Y/C | Unchanged |
| CYP4V2 | 4-187122354-GT-AC | tGT/tAC | C/Y | Unchanged |
| FAT1 | 4-187557892-GT-AC | cAC/cGT | H/R | Unchanged |
| FAT1 | 4-187557893-TG-CA | CAc/TGc | H/C | Changed missense |
| SDHA | 5-228312-GAT-AAC | GaT/AaC | D/N | Unchanged |
| SDHA | 5-256509-GT-AC | GTc/ACc | V/T | Changed missense |
| SLC12A7 | 5-1063998-GT-AC | tAC/tGT | Y/C | Unchanged |
| CCT5 | 5-10256160-GT-AC | cGT/cAC | R/H | Unchanged |
| DNAH5 | 5-13701525-TG-CA | aCA/aTG | T/M | Changed missense |
| DNAH5 | 5-13811775-TG-CA | CAa/TGa | Q/* | Unchanged |
| DNAH5 | 5-13865980-TG-CA | aCA/aTG | T/M | Changed missense |
| DROSHA | 5-31423007-TG-CA | cCA/cTG | P/L | Unchanged |
| ADAMTS12 | 5-33614399-TG-CA | CAg/TGg | Q/W | Rescued nonsense |
| SLC45A2 | 5-33954511-TG-CA | aCA/aTG | T/M | Changed missense |
| EGFLAM | 5-38370537-TG-CA | TGg/CAg | W/Q | Rescued nonsense |
| MROH2B | 5-41000343-GT-AC | tAC/tGT | Y/C | Unchanged |
| MROH2B | 5-41049389-TG-CA | aCA/aTG | T/M | Changed missense |
| MROH2B | 5-41057374-TG-CA | CAg/TGg | Q/W | Rescued nonsense |
| ITGA2 | 5-52374657-GT-CA | GTg/CAg | V/Q | Changed missense |
| RP11-45H22.3 | 5-54252345-TG-CA | aCA/aTG | T/M | Changed missense |
| ADAMTS6 | 5-64748681-TG-CA | cCA/cTG | P/L | Unchanged |
| BDP1 | 5-70806958-GT-AC | GTg/ACg | V/T | Changed missense |
| TNPO1 | 5-72183986-TG-CA | gTG/gCA | V/A | Unchanged |
| ARHGEF28 | 5-73072354-TG-CA | TGg/CAg | W/Q | Rescued nonsense |
| ENC1 | 5-73931246-TG-CA | tCA/tTG | S/L | Unchanged |
| HEXB | 5-73981270-TG-CA | tTG/tCA | L/S | Unchanged |
| F2RL1 | 5-76128521-GT-AC | aGT/aAC | S/N | Unchanged |
| PJA2 | 5-108713982-TG-CA | gCA/gTG | A/V | Unchanged |
| PJA2 | 5-108714298-TG-CA | CAa/TGa | Q/* | Unchanged |
| TSSK1B | 5-112769739-GAT-CAA | AtC/TtG | I/L | Changed missense |
| AQPEP | 5-115298705-GT-CA | GTg/CAg | V/Q | Changed missense |
| CTXN3 | 5-126993248-TG-AC | gTG/gAC | V/D | Changed missense |
| RAPGEF6 | 5-130766662-TG-CA | CAg/TGg | Q/W | Rescued nonsense |
| CTC-432M15.3 | 5-130766662-TG-CA | CAg/TGg | Q/W | Rescued nonsense |
| IRF1 | 5-131822055-TG-CA | cCA/cTG | P/L | Unchanged |
| GDF9 | 5-132198199-GGT-AGC | AcC/GcT | T/A | Unchanged |
| SEC24A | 5-134032907-GT-CA | aGT/aCA | S/T | Partially changed missense |
| KDM3B | 5-137754694-GT-AC | gGT/gAC | G/D | Unchanged |
| TMEM173 | 5-138857919-TG-CA | CAt/TGt | H/C | Changed missense |
| WDR55 | 5-140048267-TG-CA | TGt/CAt | C/H | Changed missense |
| PCDHA1 | 5-140166017-GTT-ATC | GtT/AtC | V/I | Unchanged |
| PCDHA3 | 5-140181890-GCT-ACC | GcT/AcC | A/T | Unchanged |
| PCDHA8 | 5-140221139-GT-AC | aGT/aAC | S/N | Unchanged |
| PCDHA11 | 5-140250252-TTG-CTA | TtG/CtA | L | Unchanged |
| PCDHA13 | 5-140263420-TTG-CTA | TtG/CtA | L | Unchanged |
| PCDHA13 | 5-140263444-TTG-CTA | TtG/CtA | L | Unchanged |
| PCDHB3 | 5-140481840-GT-AC | cGT/cAC | R/H | Unchanged |
| PCDHB5 | 5-140517174-TCG-CCA | TcG/CcA | S/P | Unchanged |
| PCDHB8 | 5-140559101-GAT-AAC | GaT/AaC | D/N | Unchanged |
| PCDHB16 | 5-140563980-GGT-AGC | GgT/AgC | G/S | Unchanged |
| PCDHB10 | 5-140574355-GCT-ACC | GcT/AcC | A/T | Unchanged |
| PCDHGA11 | 5-140802839-TG-CA | cTG/cCA | L/P | Unchanged |
| PCDH12 | 5-141325249-TG-CA | cCA/cTG | P/L | Unchanged |
| SH3RF2 | 5-145439647-TG-CA | TGg/CAg | W/Q | Rescued nonsense |
| SPINK5 | 5-147510865-GT-AC | gGT/gAC | G/D | Unchanged |
| SH3TC2 | 5-148386525-TG-CA | cCA/cTG | P/L | Unchanged |
| TIGD6 | 5-149374932-TG-CA | CAg/TGg | Q/W | Rescued nonsense |
| CSF1R | 5-149450132-TG-CA | CAc/TGc | H/C | Changed missense |
| PDGFRB | 5-149495395-TG-CA | cCA/cTG | P/L | Unchanged |
| ARSI | 5-149677434-GT-AC | tAC/tGT | Y/C | Unchanged |
| FAT2 | 5-150946132-GAT-AAC | AtC/GtT | I/V | Unchanged |
| HAVCR1 | 5-156479539-GT-AC | ACa/GTa | T/V | Changed missense |
| THG1L | 5-157164962-TG-CA | cTG/cCA | L/P | Unchanged |
| CCNJL | 5-159686502-TG-CA | CAt/TGt | H/C | Changed missense |
| RARS | 5-167913510-GT-AC | GTa/ACa | V/T | Changed missense |
| STK10 | 5-171520554-TG-CA | cCA/cTG | P/L | Unchanged |
| SH3PXD2B | 5-171789779-GCT-ACC | AgC/GgT | S/G | Unchanged |
| AC027309.1 | 5-172036332-TG-CA | aCA/aTG | T/M | Changed missense |
| DRD1 | 5-174868840-TG-CA | tCA/tTG | S/L | Unchanged |
| CDHR2 | 5-176004476-TG-CA | gTG/gCA | V/A | Unchanged |
| B4GALT7 | 5-177031347-GT-AC | cGT/cAC | R/H | Unchanged |
| COL23A1 | 5-177683376-GT-AC | ACg/GTg | T/V | Changed missense |
| ZNF354B | 5-178293284-TGG-CGA | TgG/CgA | W/R | Rescued nonsense |
| AC136604.1 | 5-179078989-TG-CA | TGt/CAt | C/H | Changed missense |
| HUS1B | 6-656343-TG-CA | CAa/TGa | Q/* | Unchanged |
| PSMG4 | 6-3264487-TG-CA | TGg/CAg | W/Q | Rescued nonsense |
| SLC22A23 | 6-3287118-TG-CA | tCA/tTG | S/L | Unchanged |
| RPP40 | 6-4996265-TG-CA | aCA/aTG | T/M | Changed missense |
| MCUR1 | 6-13801614-GT-AC | ACt/GTt | T/V | Changed missense |
| MCUR1 | 6-13814338-GCT-ACC | AgC/GgT | S/G | Unchanged |
| GPLD1 | 6-24437445-GT-AC | ACa/GTa | T/V | Changed missense |
| HIST1H3C | 6-26045640-TG-CA | aTG/aCA | M/T | Unchanged |
| BTN2A1 | 6-26468434-TG-CA | cTG/cCA | L/P | Unchanged |
| GNL1 | 6-30515043-TG-CA | gCA/gTG | A/V | Unchanged |
| VARS2 | 6-30888161-TG-CA | TGg/CAg | W/Q | Rescued nonsense |
| MUC21 | 6-30955099-GT-AC | GTg/ACg | V/T | Changed missense |
| MUC22 | 6-30993738-TG-CA | aTG/aCA | M/T | Partially changed missense |
| MUC22 | 6-30994878-TG-CA | aTG/aCA | M/T | Partially changed missense |
| HLA-C | 6-31237771-GGT-AGC | AcC/GcT | T/A | Unchanged |
| HLA-B | 6-31324200-GCT-CCA | AgC/TgG | S/W | Changed missense |
| MICA | 6-31378358-TG-CA | TGg/CAg | W/Q | Rescued nonsense |
| MICA | 6-31379118-GT-AC | GTa/ACa | V/T | Changed missense |
| MICA | 6-31380194-TG-CA | TGt/CAt | C/H | Changed missense |
| PRRC2A | 6-31594626-GGT-CGA | GgT/CgA | G/R | Unchanged |
| ABHD16A | 6-31657413-TAG-CAA | CtA/TtG | L | Unchanged |
| HSPA1L | 6-31778272-GT-AC | ACg/GTg | T/V | Changed missense |
| SLC44A4 | 6-31838713-GT-AC | tAC/tGT | Y/C | Unchanged |
| NOTCH4 | 6-32188642-TG-CA | cCA/cTG | P/L | Unchanged |
| HLA-DRB5 | 6-32486437-TG-CA | CAg/TGg | Q/W | Rescued nonsense |
| HLA-DRB5 | 6-32487214-TCG-CCA | CgA/TgG | R/W | Rescued nonsense |
| HLA-DRB5 | 6-32487277-TG-CA | aCA/aTG | T/M | Changed missense |
| HLA-DRB5 | 6-32489731-GT-CA | tAC/tTG | Y/L | Rescued nonsense |
| HLA-DRB5 | 6-32489939-TG-CA | CAg/TGg | Q/W | Rescued nonsense |
| HLA-DQA1 | 6-32609105-GT-AC | tGT/tAC | C/Y | Unchanged |
| HLA-DQB1 | 6-32629129-TG-CA | CAg/TGg | Q/W | Rescued nonsense |
| TAP1 | 6-32814974-GT-AC | gAC/gGT | D/G | Unchanged |
| HLA-DPA1 | 6-33036959-GT-AC | aAC/aGT | N/S | Unchanged |
| HLA-DPA1 | 6-33037419-GT-AC | aAC/aGT | N/S | Unchanged |
| WDR46 | 6-33256470-GT-AC | ACc/GTc | T/V | Changed missense |
| TAPBP | 6-33272972-GT-AC | ACt/GTt | T/V | Changed missense |
| RPS10 | 6-34392586-TG-AC | CAg/GTg | Q/V | Changed missense |
| RPS10-NUDT3 | 6-34392586-TG-AC | CAg/GTg | Q/V | Changed missense |
| TCP11 | 6-35086275-TG-CA | CAg/TGg | Q/W | Rescued nonsense |
| PNPLA1 | 6-36275458-TCG-CCA | TcG/CcA | S/P | Unchanged |
| DNAH8 | 6-38750810-GGT-AGC | GgT/AgC | G/S | Unchanged |
| DNAH8 | 6-38976712-TG-CA | cTG/cCA | L/P | Unchanged |
| TREML4 | 6-41196605-TG-CA | TGg/CAg | W/Q | Rescued nonsense |
| TFEB | 6-41652604-TG-CA | cCA/cTG | P/L | Unchanged |
| TFEB | 6-41652604-TGG-CGA | CcA/TcG | P/S | Unchanged |
| CUL7 | 6-43014298-TTG-CTA | CaA/TaG | Q/* | Unchanged |
| CUL7 | 6-43014299-TG-CA | CAa/TGa | Q/* | Unchanged |
| DLK2 | 6-43418733-GT-AC | cAC/cGT | H/R | Unchanged |
| AARS2 | 6-44275009-GAT-AAC | AtC/GtT | I/V | Unchanged |
| SLC25A27 | 6-46623696-GCT-ACC | GcT/AcC | A/T | Unchanged |
| GPR116 | 6-46826844-TG-CA | aCA/aTG | T/M | Changed missense |
| GPR115 | 6-47684573-TG-CA | aTG/aCA | M/T | Partially changed missense |
| GLYATL3 | 6-49479750-TG-CA | aTG/aCA | M/T | Partially changed missense |
| PKHD1 | 6-51483961-TG-CA | CAa/TGa | Q/* | Unchanged |
| ICK | 6-52878637-TG-CA | cCA/cTG | P/L | Unchanged |
| KLHL31 | 6-53519855-TAG-CAA | CtA/TtG | L | Unchanged |
| GFRAL | 6-55266625-TCG-CCA | TcG/CcA | S/P | Unchanged |
| BMP5 | 6-55639028-GT-AC | aAC/aGT | N/S | Unchanged |
| RP11-257K9.8 | 6-73935134-TG-AC | aCA/aGT | T/S | Changed missense |
| IMPG1 | 6-76744342-TG-CA | CAg/TGg | Q/W | Rescued nonsense |
| SNAP91 | 6-84303342-TG-CA | cCA/cTG | P/L | Unchanged |
| ZNF292 | 6-87969480-GT-AC | GTa/ACa | V/T | Changed missense |
| MDN1 | 6-90482395-GAT-AAC | AtC/GtT | I/V | Unchanged |
| FAXC | 6-99771540-TG-CA | aCA/aTG | T/M | Changed missense |
| AIM1 | 6-106967778-TG-CA | TGc/CAc | C/H | Changed missense |
| PPIL6 | 6-109752450-GT-AC | cAC/cGT | H/R | Unchanged |
| MICAL1 | 6-109768295-GT-AC | tAC/tGT | Y/C | Unchanged |
| SLC22A16 | 6-110778128-TG-CA | CAt/TGt | H/C | Changed missense |
| PTPRK | 6-128404875-GAT-AAC | AtC/GtT | I/V | Unchanged |
| TMEM200A | 6-130761796-GCT-ACC | GcT/AcC | A/T | Unchanged |
| CTAGE9 | 6-132030028-TG-CA | aCA/aTG | T/M | Changed missense |
| ECT2L | 6-139170460-GT-AC | GTa/ACa | V/T | Changed missense |
| HIVEP2 | 6-143074700-TG-CA | cCA/cTG | P/L | Unchanged |
| ULBP1 | 6-150290446-TG-CA | aTG/aCA | M/T | Partially changed missense |
| IYD | 6-150716696-TG-CA | TGc/CAc | C/H | Changed missense |
| ESR1 | 6-152201874-GT-AC | cGT/cAC | R/H | Unchanged |
| SYNJ2 | 6-158497715-GTT-ATC | GtT/AtC | V/I | Unchanged |
| PLG | 6-161159625-GCT-ACC | GcT/AcC | A/T | Unchanged |
| PDE10A | 6-165895824-TG-CA | tCA/tTG | S/L | Unchanged |
| RPS6KA2 | 6-167271710-GT-AC | ACc/GTc | T/V | Changed missense |
| RP11-514O12.4 | 6-167271710-GT-AC | ACc/GTc | T/V | Changed missense |
| UNC93A | 6-167728774-TG-CA | aTG/aCA | M/T | Partially changed missense |
| THBS2 | 6-169648767-GT-AC | aAC/aGT | N/S | Unchanged |
| WDR27 | 6-169972595-GT-AC | cAC/cGT | H/R | Unchanged |
| WDR27 | 6-169972596-TG-CA | CAc/TGc | H/C | Changed missense |
| HEATR2 | 7-810218-GT-AC | GTg/ACg | V/T | Changed missense |
| TMEM184A | 7-1586660-GCT-ACC | AgC/GgT | S/G | Unchanged |
| SNX8 | 7-2303986-TG-CA | gCA/gTG | A/V | Unchanged |
| CARD11 | 7-2946461-TCG-CCA | CgA/TgG | R/W | Rescued nonsense |
| PAPOLB | 7-4900341-GCT-ACC | AgC/GgT | S/G | Unchanged |
| COL28A1 | 7-7413149-TG-CA | cCA/cTG | P/L | Unchanged |
| THSD7A | 7-11521542-TG-CA | cCA/cTG | P/L | Unchanged |
| THSD7A | 7-11581134-TG-CA | gCA/gTG | A/V | Unchanged |
| AGMO | 7-15458186-TG-CA | aCA/aTG | T/M | Changed missense |
| STEAP1B | 7-22533235-TG-CA | CAt/TGt | H/C | Changed missense |
| DFNA5 | 7-24742436-TG-CA | gCA/gTG | A/V | Unchanged |
| C7orf31 | 7-25181912-TG-CA | CAt/TGt | H/C | Changed missense |
| SCRN1 | 7-29966141-TG-CA | CAg/TGg | Q/W | Rescued nonsense |
| NME8 | 7-37907304-TG-CA | TGt/CAt | C/H | Changed missense |
| SPDYE1 | 7-44047066-TG-CA | TGc/CAc | C/H | Changed missense |
| MYO1G | 7-45004063-TG-CA | CAg/TGg | Q/W | Rescued nonsense |
| MYO1G | 7-45009723-GT-AC | aAC/aGT | N/S | Unchanged |
| NACAD | 7-45120248-GT-AC | aAC/aGT | N/S | Unchanged |
| PKD1L1 | 7-47869038-TG-CA | gCA/gTG | A/V | Unchanged |
| C7orf57 | 7-48086161-TG-CA | aTG/aCA | M/T | Partially changed missense |
| C7orf57 | 7-48092484-GAT-AAC | GaT/AaC | D/N | Unchanged |
| GRB10 | 7-50673029-TG-CA | gCA/gTG | A/V | Unchanged |
| ZNF479 | 7-57188526-TG-CA | CAc/TGc | H/C | Changed missense |
| ZNF680 | 7-64004108-TG-CA | CAg/TGg | Q/W | Rescued nonsense |
| ERV3-1 | 7-64452532-TG-CA | tCA/tTG | S/L | Unchanged |
| ERV3-1 | 7-64452541-GT-AC | cAC/cGT | H/R | Unchanged |
| MLXIPL | 7-73020301-TG-CA | tCA/tTG | S/L | Unchanged |
| WBSCR28 | 7-73279482-TG-CA | TGg/CAg | W/Q | Rescued nonsense |
| TRIM73 | 7-75028240-TG-CA | cTG/cCA | L/P | Unchanged |
| POM121C | 7-75051375-TG-CA | cCA/cTG | P/L | Unchanged |
| SRCRB4D | 7-76028057-GT-AC | ACg/GTg | T/V | Changed missense |
| ZP3 | 7-76069811-TCG-CCA | TcG/CcA | S/P | Unchanged |
| UPK3B | 7-76144560-TG-CA | TGg/CAg | W/Q | Rescued nonsense |
| UPK3B | 7-76144560-TGG-CGA | TgG/CgA | W/R | Rescued nonsense |
| SEMA3C | 7-80374438-TG-CA | cCA/cTG | P/L | Unchanged |
| SEMA3A | 7-83590852-TG-CA | aCA/aTG | T/M | Changed missense |
| C7orf63 | 7-89912301-GT-AC | GTg/ACg | V/T | Changed missense |
| KRIT1 | 7-91864193-TG-CA | cCA/cTG | P/L | Unchanged |
| MCM7 | 7-99693078-GAT-AAC | AtC/GtT | I/V | Unchanged |
| MEPCE | 7-100028483-GT-AC | aGT/aAC | S/N | Unchanged |
| ZAN | 7-100377082-TG-CA | cTG/cCA | L/P | Unchanged |
| ZAN | 7-100386900-TG-CA | TGg/CAg | W/Q | Rescued nonsense |
| MUC12 | 7-100634706-TCG-ACC | TcG/AcC | S/T | Unchanged |
| MUC12 | 7-100635940-TG-CA | aTG/aCA | M/T | Partially changed missense |
| MUC17 | 7-100676793-TG-CA | cTG/cCA | L/P | Unchanged |
| MUC17 | 7-100683929-GT-AC | GTc/ACc | V/T | Changed missense |
| MUC17 | 7-100684686-TG-CA | aTG/aCA | M/T | Partially changed missense |
| MYL10 | 7-101256833-TG-CA | gCA/gTG | A/V | Unchanged |
| FBXL13 | 7-102462636-TG-CA | gCA/gTG | A/V | Unchanged |
| FBXL13 | 7-102669807-GT-AC | ACt/GTt | T/V | Changed missense |
| NAMPT | 7-105903904-TG-CA | tCA/tTG | S/L | Unchanged |
| LAMB1 | 7-107593989-TG-CA | CAg/TGg | Q/W | Rescued nonsense |
| LAMB4 | 7-107738882-GT-AC | tAC/tGT | Y/C | Unchanged |
| LRRN3 | 7-110763561-GAT-AAC | GaT/AaC | D/N | Unchanged |
| SLC13A1 | 7-122809233-GT-AC | aAC/aGT | N/S | Unchanged |
| FLNC | 7-128487864-GAT-AAC | GaT/AaC | D/N | Unchanged |
| STRIP2 | 7-129107227-TG-CA | TGc/CAc | C/H | Changed missense |
| PODXL | 7-131190696-TG-CA | gCA/gTG | A/V | Unchanged |
| PLXNA4 | 7-131864591-TG-CA | gCA/gTG | A/V | Unchanged |
| CNOT4 | 7-135047703-TG-CA | aCA/aTG | T/M | Changed missense |
| NUP205 | 7-135269655-TG-CA | aTG/aCA | M/T | Partially changed missense |
| DGKI | 7-137150695-TG-CA | aCA/aTG | T/M | Changed missense |
| SVOPL | 7-138305806-TG-CA | gCA/gTG | A/V | Unchanged |
| KIAA1549 | 7-138596064-GT-AC | tAC/tGT | Y/C | Unchanged |
| DENND2A | 7-140258000-TG-CA | tCA/tTG | S/L | Unchanged |
| TAS2R38 | 7-141672602-GAT-AAC | AtC/GtT | I/V | Unchanged |
| TRBV5-6 | 7-142131851-TG-CA | gCA/gTG | A/V | Unchanged |
| TRPV5 | 7-142625249-TG-CA | aCA/aTG | T/M | Changed missense |
| TRPV5 | 7-142625882-GT-AC | tAC/tGT | Y/C | Unchanged |
| TAS2R60 | 7-143141474-GT-AC | cGT/cAC | R/H | Unchanged |
| FAM115C | 7-143417083-TG-CA | TGt/CAt | C/H | Changed missense |
| OR2A25 | 7-143771527-GT-AC | tGT/tAC | C/Y | Unchanged |
| ZNF212 | 7-148947470-TG-CA | aTG/aCA | M/T | Partially changed missense |
| ACTR3C | 7-149983565-TG-CA | CAg/TGg | Q/W | Rescued nonsense |
| RP1L1 | 8-10467627-TG-CA | aCA/aTG | T/M | Changed missense |
| RP1L1 | 8-10467628-GT-AC | ACa/GTa | T/V | Changed missense |
| RP1L1 | 8-10469340-GTT-ATC | AaC/GaT | N/D | Unchanged |
| FAM86B1 | 8-12044328-TG-CA | tCA/tTG | S/L | Unchanged |
| FAM86B2 | 8-12286609-TG-CA | tCA/tTG | S/L | Unchanged |
| CSGALNACT1 | 8-19316086-GT-AC | cAC/cGT | H/R | Unchanged |
| SLC39A14 | 8-22262321-TG-CA | cTG/cCA | L/P | Unchanged |
| ENTPD4 | 8-23291835-TG-CA | cCA/cTG | P/L | Unchanged |
| EBF2 | 8-25708267-TG-CA | tCA/tTG | S/L | Unchanged |
| NUGGC | 8-27918057-TG-CA | CAa/TGa | Q/* | Unchanged |
| NUGGC | 8-27925202-GCT-ACC | AgC/GgT | S/G | Unchanged |
| WRN | 8-31024654-TG-CA | TGt/CAt | C/H | Changed missense |
| RB1CC1 | 8-53569177-GT-CA | ACt/TGt | T/C | Changed missense |
| OPRK1 | 8-54142157-TG-CA | gCA/gTG | A/V | Unchanged |
| ADHFE1 | 8-67380528-TG-CA | TGt/CAt | C/H | Changed missense |
| CPA6 | 8-68346339-TG-CA | gCA/gTG | A/V | Unchanged |
| TRPA1 | 8-72936145-TG-CA | CAt/TGt | H/C | Changed missense |
| ZFHX4 | 8-77745633-TCG-CCA | TcG/CcA | S/P | Unchanged |
| NBN | 8-90958422-TG-CA | cCA/cTG | P/L | Unchanged |
| KIAA1429 | 8-95504024-TG-CA | CAg/TGg | Q/W | Rescued nonsense |
| KIAA1429 | 8-95531419-TTG-CTA | CaA/TaG | Q/* | Unchanged |
| CCNE2 | 8-95894448-GT-AC | ACt/GTt | T/V | Changed missense |
| SPAG1 | 8-101252680-TG-CA | aTG/aCA | M/T | Partially changed missense |
| LRP12 | 8-105511684-TG-CA | aCA/aTG | T/M | Changed missense |
| RP11-422N16.3 | 8-110657514-GT-AC | GTa/ACa | V/T | Changed missense |
| TAF2 | 8-120790304-TG-CA | aCA/aTG | T/M | Changed missense |
| DSCC1 | 8-120847188-TG-CA | CAt/TGt | H/C | Changed missense |
| TRMT12 | 8-125463250-TGG-CGA | TgG/CgA | W/R | Rescued nonsense |
| TG | 8-134108546-TG-CA | TGg/CAg | W/Q | Rescued nonsense |
| ST3GAL1 | 8-134488007-GGT-AGC | AcC/GcT | T/A | Unchanged |
| COL22A1 | 8-139697478-TG-CA | cCA/cTG | P/L | Unchanged |
| SLC45A4 | 8-142222443-GTT-ATC | AaC/GaT | N/D | Unchanged |
| SLC45A4 | 8-142228809-TG-CA | gCA/gTG | A/V | Unchanged |
| LYPD2 | 8-143832509-GT-AC | aAC/aGT | N/S | Unchanged |
| C8orf31 | 8-144124609-TG-CA | cTG/cCA | L/P | Unchanged |
| RHPN1 | 8-144462313-TG-CA | cTG/cCA | L/P | Unchanged |
| MROH6 | 8-144649625-TCG-CCA | CgA/TgG | R/W | Rescued nonsense |
| TIGD5 | 8-144680371-TGG-CGA | TgG/CgA | W/R | Rescued nonsense |
| SCRIB | 8-144874554-TG-CA | cCA/cTG | P/L | Unchanged |
| PLEC | 8-144992103-TG-CA | aCA/aTG | T/M | Changed missense |
| PLEC | 8-144992361-TG-CA | cCA/cTG | P/L | Unchanged |
| PLEC | 8-145001031-TG-CA | CAc/TGc | H/C | Changed missense |
| GPAA1 | 8-145138621-TG-CA | gTG/gCA | V/A | Unchanged |
| CYHR1 | 8-145689915-GT-AC | cAC/cGT | H/R | Unchanged |
| DMRT3 | 9-990712-TTG-CTA | TtG/CtA | L | Unchanged |
| PTPRD | 9-8518143-TG-CA | gCA/gTG | A/V | Unchanged |
| MPDZ | 9-13219570-TG-CA | aCA/aTG | T/M | Changed missense |
| SLC24A2 | 9-19622268-TG-CA | cCA/cTG | P/L | Unchanged |
| IFNA14 | 9-21239650-GTT-ATC | AaC/GaT | N/D | Unchanged |
| DDX58 | 9-32487521-TG-CA | aCA/aTG | T/M | Changed missense |
| FAM205A | 9-34724580-TG-CA | CAt/TGt | H/C | Changed missense |
| FAM205A | 9-34726418-TG-CA | cCA/cTG | P/L | Unchanged |
| PIGO | 9-35091950-GT-CA | ACa/TGa | T/* | Gained nonsense |
| OR13J1 | 9-35870000-GT-AC | cAC/cGT | H/R | Unchanged |
| OR13J1 | 9-35870001-TG-CA | CAc/TGc | H/C | Changed missense |
| PAX5 | 9-36840623-GT-AC | tAC/tGT | Y/C | Unchanged |
| FRMPD1 | 9-37740635-GCT-ACC | GcT/AcC | A/T | Unchanged |
| ALDH1B1 | 9-38396502-GT-AC | GTg/ACg | V/T | Changed missense |
| ANKRD18A | 9-38575611-TG-CA | aCA/aTG | T/M | Changed missense |
| FOXD4L6 | 9-69200917-GT-AC | cAC/cGT | H/R | Unchanged |
| FOXD4L5 | 9-70177441-GT-AC | tAC/tGT | Y/C | Unchanged |
| SMC5 | 9-72897440-TG-CA | TGt/CAt | C/H | Changed missense |
| PRUNE2 | 9-79324179-GT-AC | ACg/GTg | T/V | Changed missense |
| DAPK1 | 9-90322023-GT-AC | aGT/aAC | S/N | Unchanged |
| SHC3 | 9-91656963-TG-CA | cCA/cTG | P/L | Unchanged |
| NUTM2F | 9-97081356-TG-CA | aCA/aTG | T/M | Changed missense |
| PTCH1 | 9-98220526-GT-AC | aAC/aGT | N/S | Unchanged |
| TBC1D2 | 9-100971348-TG-CA | gCA/gTG | A/V | Unchanged |
| GRIN3A | 9-104449294-GT-AC | aAC/aGT | N/S | Unchanged |
| OR13C5 | 9-107361449-TAG-CAA | CtA/TtG | L | Unchanged |
| AKAP2 | 9-112900199-TG-CA | tTG/tCA | L/S | Unchanged |
| PALM2-AKAP2 | 9-112900199-TG-CA | tTG/tCA | L/S | Unchanged |
| SVEP1 | 9-113169630-TG-CA | gCA/gTG | A/V | Unchanged |
| C9orf84 | 9-114490308-TG-CA | CAt/TGt | H/C | Changed missense |
| C9orf147 | 9-115249262-TG-CA | cCA/cTG | P/L | Unchanged |
| RNF183 | 9-116060124-TG-CA | CAa/TGa | Q/* | Unchanged |
| DFNB31 | 9-117166338-GT-CA | cAC/cTG | H/L | Partially changed missense |
| TNFSF8 | 9-117666499-GT-AC | tAC/tGT | Y/C | Unchanged |
| TNC | 9-117804544-TG-CA | aCA/aTG | T/M | Changed missense |
| TNC | 9-117840328-GT-CA | aAC/aTG | N/M | Changed missense |
| TNC | 9-117846580-TG-CA | CAg/TGg | Q/W | Rescued nonsense |
| TNC | 9-117848394-TG-CA | CAg/TGg | Q/W | Rescued nonsense |
| ASTN2 | 9-119495697-TG-CA | cCA/cTG | P/L | Unchanged |
| ASTN2 | 9-120053776-TG-CA | gCA/gTG | A/V | Unchanged |
| OR1B1 | 9-125391677-TG-CA | aCA/aTG | T/M | Changed missense |
| OR1K1 | 9-125563212-TG-CA | TGg/CAg | W/Q | Rescued nonsense |
| CRB2 | 9-126133547-TG-CA | gTG/gCA | V/A | Unchanged |
| DENND1A | 9-126144758-TG-CA | tCA/tTG | S/L | Unchanged |
| LMX1B | 9-129455585-TCG-ACC | TcG/AcC | S/T | Unchanged |
| FAM129B | 9-130289485-GT-AC | tAC/tGT | Y/C | Unchanged |
| TBC1D13 | 9-131565554-TG-CA | gTG/gCA | V/A | Unchanged |
| LRRC8A | 9-131670918-GT-AC | cGT/cAC | R/H | Unchanged |
| USP20 | 9-132623866-GCT-ACC | GcT/AcC | A/T | Unchanged |
| PPAPDC3 | 9-134183502-GT-AC | cGT/cAC | R/H | Unchanged |
| PRRC2B | 9-134346314-GT-AC | cGT/cAC | R/H | Unchanged |
| PRRC2B | 9-134350647-TG-CA | aTG/aCA | M/T | Partially changed missense |
| DDX31 | 9-135470412-GAT-AAC | AtC/GtT | I/V | Unchanged |
| SURF6 | 9-136199466-GT-AC | ACg/GTg | T/V | Changed missense |
| COL5A1 | 9-137658303-GT-CA | GTc/CAc | V/H | Changed missense |
| FCN2 | 9-137775154-GT-AC | cGT/cAC | R/H | Unchanged |
| PPP1R26 | 9-138377657-TG-CA | aTG/aCA | M/T | Partially changed missense |
| OBP2A | 9-138440641-TG-CA | aTG/aCA | M/T | Partially changed missense |
| GLT6D1 | 9-138516189-GT-AC | cAC/cGT | H/R | Unchanged |
| UBAC1 | 9-138838140-TG-CA | gCA/gTG | A/V | Unchanged |
| QSOX2 | 9-139100805-TG-CA | cCA/cTG | P/L | Unchanged |
| CCDC183 | 9-139693596-TG-CA | aTG/aCA | M/T | Partially changed missense |
| RP11-216L13.17 | 9-139693596-TG-CA | aTG/aCA | M/T | Partially changed missense |
| CCDC183 | 9-139700605-TG-CA | TGg/CAg | W/Q | Rescued nonsense |
| ABCA2 | 9-139903219-GAT-CAA | AtC/TtG | I/L | Changed missense |
| CSF2RA | X-1419454-TG-CA | gTG/gCA | V/A | Unchanged |
| ASMTL | X-1554649-TG-CA | aCA/aTG | T/M | Changed missense |
| DHRSX | X-2139200-TG-CA | CAt/TGt | H/C | Changed missense |
| WWC3 | X-10085226-GT-AC | cGT/cAC | R/H | Unchanged |
| FTHL17 | X-31089712-TG-CA | CAg/TGg | Q/W | Rescued nonsense |
| FAM47B | X-34961339-TTG-CTA | TtG/CtA | L | Unchanged |
| FAAH2 | X-57405162-GT-AC | cGT/cAC | R/H | Unchanged |
| HEPH | X-65382685-TG-CA | gTG/gCA | V/A | Unchanged |
| ABCB7 | X-74284996-TG-CA | gCA/gTG | A/V | Unchanged |
| ZMAT1 | X-101138792-TG-CA | CAg/TGg | Q/W | Rescued nonsense |
| ATP1B4 | X-119500459-TG-CA | gTG/gCA | V/A | Unchanged |
| CT47B1 | X-120008979-TGG-CGA | CcA/TcG | P/S | Unchanged |
| IGSF1 | X-130420006-TG-CA | cCA/cTG | P/L | Unchanged |
| USP26 | X-132160161-TG-CA | cCA/cTG | P/L | Unchanged |
| FAM122C | X-133941714-GT-AC | aGT/aAC | S/N | Unchanged |
| MAP7D3 | X-135310785-TG-CA | CAa/TGa | Q/* | Unchanged |
| SPANXC | X-140335857-TGG-CGA | CcA/TcG | P/S | Unchanged |
| MAGEC3 | X-140985607-TG-CA | TGt/CAt | C/H | Changed missense |
| MAGEC1 | X-140993674-GTT-CTA | GtT/CtA | V/L | Unchanged |
| SLITRK2 | X-144905744-TCG-CCA | TcG/CcA | S/P | Unchanged |
| PNMA5 | X-152158979-TG-CA | cCA/cTG | P/L | Unchanged |
| PLXNB3 | X-153041544-TG-CA | aTG/aCA | M/T | Partially changed missense |
| IL9R | X-155239682-GT-AC | GTg/ACg | V/T | Changed missense |

TABLE L
List of Exemplary Cas9 or Cas12a Orthologs

| | UniProt or | |
|---|---|---|
| | GenBank | Nickase |
| | Accession | Mutations/Catalytic |
| Ortholog | Number | residues |
| S. pyogenes Cas9 | Q99ZW2.1 | D10A, E762A, H840A, |
| (SpCas9) | | N854A, N863A, D986A¹⁷ |
| S. aureus Cas9 | J7RUA5.1 | D10A and N580¹⁸ |
| (SaCas9) | | |
| S. thermophilus Cas9 | G3ECR1.2 | D31A and N891A¹⁹ |
| (St1Cas9) | | |
| S. pasteurianus Cas9 | BAK30384.1 | D10, H599* |
| (SpaCas9) | | |
| C. jejuni Cas9 | Q0P897.1 | D8A, H559A²⁰ |
| (CjCas9) | | |
| F. novicida Cas9 | A0Q5Y3.1 | D11, N995²¹ |
| (FnCas9) | | |
| P. lavamentivorans | A7HP89.1 | D8, H601* |
| Cas9 (PlCas9) | | |
| C. lari Cas9 (ClCas9) | G1UFN3.1 | D7, H567* |
| Pasteurella multocida | Q9CLT2.1 | |
| Cas9 | | |
| F. novicida Cpf1 | A0Q7Q2.1 | D917, E1006, D1255²¹ |
| (FnCpf1) | | |
| M. bovoculi Cpf1 | WP 052585281.1 | D986A** |
| (MbCpf1) | | |
| A. sp. BV3L6 Cpf1 | U2UMQ6.1 | D908, 993E, Q1226, D1263²³ |
| (AsCpf1) | | |
| L. bacterium N2006 | A0A182DWE3.1 | D832A²⁴ |
| (LbCpf1) | | |

*predicted based on UniRule annotation on the UniProt database.
**Unpublished but deposited at addgene by Ervin Welker: pTE4565 (Addgene plasmid # 88903)

TABLE M
List of Exemplary High Fidelity and/or PAM-relaxed RGN Orthologs

| Published HF/PAM-RGN | | |
|---|---|---|
| variants | PMID | Mutations* |
| S. pyogenes Cas9 | 26628643 | K810A/K1003A/R1060A (1.0); |
| (SpCas9) eSpCasS | | K848A/K1003A/R1060A(1.1) |
| S. pyogenes Cas9 | 29431739 | M495V/Y515N/K526E/R661Q; |
| (SpCas9) evoCas9 | | (M495V/Y515N/K526E/R661S; |
| | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi Cas9 | | |
| S. pyogenes Cas9 | 28931002 | N692A, M694A, Q695A, H698A |
| (SpCas9) HypaCas9 | | |
| S. pyogenes Cas9 | 30082838 | F539S, M763I, K890N |
| (SpCas9) Sniper-Cas9 | | |
| S. pyogenes Cas9 | 29512652 | A262T, R324L, S409I, E480K, E543D, |
| (SpCas9) xCas9 | | M694I, E1219V |
| S. pyogenes Cas9 | 30166441 | R1335V, L1111R, D1135V, G1218R, |
| (SpCas9) SpCas9-NG | | E1219F, A1322R, T1337R |
| S. pyogenes Cas9 | 26098369 | D1135V, R1335Q, T1337R; |
| (SpCas9) VQR/VRER | | D1135V/G1218R/R1335E/T1337R |
| S. aureus Cas9 | 26524662 | E782K/N968K/R1015H |
| (SaCas9)-KKH | | |
| enAsCas12a | USSN | One or more of: E174R, S170R, S542R, |
| | 15/960,271 | K548R, K548V, N551R, N552R, K607R, |
| | | K607H, e.g., E174R/S542R/K548R, |
| | | E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, |
| | | S170R/S542R/K607R, or |
| | | S170R/S542R/K548V/N552R |
| enAsCas12a-HF | USSN | One or more of: E174R, S542R, K548R, |
| | 15/960,271 | e.g., E174R/S542R/K548R, |
| | | E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, |
| | | S170R/S542R/K607R, or |
| | | S170R/S542R/K548V/N552R, with the |
| | | addition of one or more of: N282A, |
| | | T315A, N515A and K949A |
| enLbCas12a(HF) | USSN | One or more of T152R, T152K, D156R, |
| | 15/960,271 | D156K, Q529K, G532R, G532K, G532Q, |
| | | K538R, K538V, D541R, Y542R, M592A, |
| | | K595R, K595H, K595S or K595Q, e.g., |
| | | D156R/G532R/K538R, |
| | | D156R/G532R/K595R, |
| | | D156R/G532R/K538V/Y542R, |
| | | T152R/G532R/K538R, T152R/D156R, |
| | | D156R/G532R, T152R/G532R, |
| | | D156R/G532R/K538R/D541R, |
| | | D156R/G532R/K595H, |
| | | T152R/G532R/K595R, |
| | | T152R/G532R/K538V/Y542R, optionally |
| | | with the addition of one or more of: |
| | | N260A, N256A, K514A, D505A, K881A, |
| | | S286A, K272A, K897A |
| enFnCas12a(HF) | USSN | One or more of T177A, K180R, K180K, |
| | 15/960,271 | E184R, E184K, T604K, N607R, N607K, |
| | | N607Q, K613R, K613V, D616R, N617R, |
| | | M668A, K671R, K671H, K671S, or K671Q, |
| | | e.g., E184R/N607R/K613R, |
| | | E184R/N607R/K671R, |
| | | E184R/N607R/K613V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, |
| | | K180R/N607R/K671R, |
| | | K180R/N607R/K613V/N617R, optionally |
| | | with the addition of one or more of: |
| | | N305A, N301A, K589A, N580A, K962A, |
| | | S334A, K320A, K978A |
| chimeric Cas9 | 30718489 | S. aureus Cas9 with PAM interaction |
| cCas9 | | domain from SaCas9 orthologues, |
| | | expands recognition and targetability |
| | | of NNVRRN, NNVACT, NNVATG, |
| | | NNVATT, NNVGCT, NNVGTG, and |
| | | NNVGTT PAM sequences |
| Streptococcus | doi: https://doi.org/10.1101/429654 | Recognizes 5′-NAA-3′ PAM |
| macacae (Smac) Cas9 | | |
| NCTC 11558 | | |
| Spy-mac Cas9, | doi: https://doi.org/10.1101/429654 | Recognizes 5′-NAA-3′ PAM |
| Smac-py Cas9 | | |
| N. meningitidis | 30581144 | Recognizes N₄CC PAM |
| Nme2Cas9 | | |
| SpG Cas9 | 32217751 | SpCas9 variant capable of targeting |
| (SEQ-ID 158) | | NGN PAMs |
| | | D1135L/S1136W/G1218K/E1219Q/ |
| | | R1335Q/T1337R |
| | | Also as SpG-HF1 in combination |
| | | with N497A/R661A/Q695A/Q926A |
| SpRY Cas9 | 32217751 | SpCas9 variant capable of targeting |
| (SEQ-ID 157) | | NRN > NYN PAMs |
| | | SpRY(A61R/L1111R/D1135L/S1136W/ |
| | | G1218K/E1219Q/N1317R/A1322R/ |
| | | R1333P/R1335Q/T1337R); also as |
| | | SpRY-HF1 in combination with |
| | | N497A/R661A/Q695A/Q926A |

*predicted based on UniRule annotation on the UniProt database.

TABLE N
Amino acid substitutions predicted to generate ABE variants
with reduced RNA editing. This table lists the residue changes
in either or both TadA domains of the TadA heterodimer (present
in e.g., ABE7.10) predicted to cause an RRE phenotype, next
to the reasoning behind the proposed changes.

| | |
|---|---|
| Residues to Change | Rationale |

| Wild type | Engineered | Protein | Binding |
|---|---|---|---|
| (WT) TadA | TadA | structure | prediction |
| S7 | S205 | | x |
| H8 | H206 | | x |
| E9 | E207 | | x |
| Y10 | Y208 | | x |
| W11 | W209 | | x |
| M12 | M210 | | x |
| R13 | R211 | x | x |
| H14 | H212 | | x |
| T17 | T215 | | x |
| K20 | K218 | x | x |
| R21 | R219 | x | x |
| W23 | R221 | x | |
| E25 | E223 | x | x |
| R26 | R224 | x | x |
| E27 | E225 | x | |
| V28 | V226 | x | x |
| P29 | P227 | | x |
| V30 | V228 | x | |
| G31 | G229 | | x |
| H36 | L234 | | x |
| N37 | N235 | | x |
| N38 | N236 | | x |
| N46 | N244 | x | |
| R47 | R245 | | x |
| P48 | A246 | x | |
| I49 | I247 | x | |
| G50 | G248 | | x |
| R51 | I249 | | x |
| H52 | H250 | | x |
| D53 | D251 | | x |
| P54 | P252 | | x |
| T55 | T253 | | x |
| A56 | A254 | x | |
| H57 | H255 | x | x |
| A58 | A256 | x | |
| E59 | E257 | x | |
| R64 | R262 | | x |
| Q65 | Q263 | | x |
| G67 | G265 | | x |
| L68 | L266 | | x |
| Q71 | Q269 | | x |
| N72 | N270 | | x |
| R74 | R272 | | x |
| I76 | I274 | | x |
| D77 | D275 | | x |
| Y81 | Y279 | | x |
| V82 | V280 | x | |
| T83 | T281 | | x |
| L84 | F282 | x | |
| E85 | E283 | x | |
| P86 | P284 | x | x |
| C87 | C285 | x | x |
| V88 | V286 | | x |
| M89 | M287 | | x |
| C90 | C288 | x | x |
| R98 | R296 | | x |
| G100 | G298 | | x |
| R101 | R299 | | x |
| A106 | V304 | x | |
| R107 | R305 | x | |
| D108 | N306 | x | |
| A109 | A307 | x | |
| K110 | K308 | x | |
| T111 | T309 | x | |
| D119 | D317 | | x |
| H122 | H320 | | x |
| H123 | Y321 | | x |
| P124 | P322 | | x |
| G125 | G323 | | x |
| M126 | M324 | | x |
| N127 | N325 | | x |
| H128 | H326 | | x |
| R129 | R327 | | x |
| V130 | V328 | | x |
| E131 | E329 | | x |
| I132 | I330 | | x |
| T133 | T331 | | x |
| E134 | E332 | | x |
| G135 | G333 | | x |
| L137 | L335 | | x |
| A138 | A336 | x | x |
| D139 | D337 | | x |
| E140 | E338 | | x |
| C141 | C339 | x | x |
| A142 | A340 | x | x |
| A143 | A341 | x | x |
| L144 | L342 | | x |
| L145 | L343 | x | x |
| S146 | C344 | x | |
| D147 | Y345 | | x |
| F148 | F346 | x | x |
| F149 | F347 | x | x |
| R150 | R348 | x | x |
| M151 | M349 | | x |
| R152 | P350 | x | x |
| R153 | R351 | x | |
| Q154 | Q352 | | x |
| E155 | V353 | x | x |
| I156 | F354 | | x |
| K157 | N355 | | x |
| K160 | K358 | | x |
| K161 | K359 | | x |

TABLE O
Amino acid residues whose mutation may be expected to
yield base editor RRE variants. These positions were
chosen based on a APOBEC1 structural model and RNA/DNA
binding predictions or based on previous description
in the literature as residues whose mutation reduced
the RNA editing or binding activities of isolated APOBEC1.

| Residue Change | Reasoning |
|---|---|
| E24, V25 | model & RNA binding prediction |
| R118, Y120, H121, R126 | model & RNA binding prediction |
| W224-K229 | model & RNA binding prediction |
| P168-I186 | model & RNA binding prediction |
| L173 + L180 | model & RNA binding prediction |
| R15, R16, R17, to K15-17 & A15-17 | Teng et al, J Lipid Research 1999 |
| Deletion E181-L210 | Teng et al, J Lipid Research 1999 |
| P190 + P191 | Teng et al, J Lipid Research 1999 |
| Deletion L210-K229 (C-terminal) | Teng et al, J Lipid Research 1999 |
| Deletion S2-L14 (N-terminal) | Teng et al, J Lipid Research 1999 |
| V64, F66 | Teng et al, J Lipid Research 1999 |
| L180A | Teng et al, J Lipid Research 1999 |
| C192, L193, L196, P201, L203, | Teng et al, J Lipid Research 1999 |
| L210, P219, P220 | |
| P92 | MacGinnitie et al, JBC 1995 |

TABLE P
Summed frequency (%) of alleles with concurrent A-to-G and C-
to-T SPACE editing of 4 replicates using 28 gRNAs (for FIG. 4)

| | Summed frequency (%) of alleles with concurrent | | | |
|---|---|---|---|---|
| | A-to-G and C-to-T SPACE editing | | | |
| | Rep. 1 | Rep. 2 | Rep. 3 | Rep. 4 |
| HEK site 2 (ABE | 68.7% | 64.1% | 57.0% | 55.3% |
| site 1) | | | | |
| ABE site 2 | 6.8% | 4.7% | 5.0% | 3.5% |
| ABE site 3 | 17.4% | 15.6% | 19.5% | 14.8% |
| ABE site 4 | 53.4% | 58.1% | 59.2% | 47.4% |
| ABE site 7 | 11.7% | 12.6% | 11.8% | 12.6% |
| ABE site 8 | 6.8% | 7.2% | 11.2% | 6.4% |
| ABE site 9 | 20.3% | 24.7% | 25.8% | 19.2% |
| ABE site 10 | 20.1% | 23.8% | 16.8% | 20.0% |
| ABE site 11 | 46.9% | 51.8% | 42.2% | 40.6% |
| ABE site 14 | 35.2% | 37.7% | 31.4% | 30.5% |
| ABE site 15 | 40.3% | 44.9% | 36.4% | 38.4% |
| ABE site 17 | 39.0% | 35.7% | 34.3% | 36.9% |
| ABE site 18 | 28.8% | 28.1% | 20.2% | 21.9% |
| ABE site 19 | 6.6% | 15.4% | 20.1% | 27.1% |
| EMX1 site 1 | 0.0% | 0.0% | 0.0% | 0.0% |
| EMX1 site 2 | 11.7% | 9.2% | 8.5% | 8.9% |
| FANCF site 1 | 5.0% | 5.5% | 4.7% | 4.4% |
| HEK site 3 | 16.9% | 14.5% | 14.5% | 18.4% |
| HEK site 4 | 24.8% | 39.9% | 37.2% | 31.5% |
| PDCD1 site 1 | 14.0% | 15.5% | 18.3% | 12.7% |
| PPP1R12C site 2 | 22.0% | 28.7% | 27.6% | 25.9% |
| PPP1R12C site 3 | 41.5% | 36.0% | 33.3% | 32.2% |
| PPP1R12C site 4 | 0.0% | 0.0% | 0.0% | 0.0% |
| PPP1R12C site 5 | 2.3% | 0.0% | 0.0% | 0.0% |
| PPP1R12C site 6 | 15.6% | 20.3% | 13.6% | 16.4% |
| PPP1R12C site 7 | 0.0% | 0.0% | 0.0% | 0.0% |
| RNF2 site 1 | 9.5% | 10.3% | 10.2% | 14.6% |
| VEGFA site 4 | 20.7% | 36.3% | 39.4% | 28.3% |

* only alleles with ≥1% frequency with edits in the editing window are counted

TABLE Q
RNA off-target editing induced by ABEmax, miniABEmax-V82G, Target-AID, and SPACE with
gRNAs targeting HEK site 2 and RNF2 site 1, determined by RNA-seq (for FIG. 11)

| | | | | | | | | | | | | Mean | Median |
|---|---|---|---|---|---|---|---|---|---|---|---|---|---|
| | | | | | | | | A-to-I | | C-to-U | | Editing | Editing |
| Cell | BE | gRNA | Sort | EJUL | | Replicate | A-to-I | (%) | C-to-U | (%) | Other | Efficiency | Efficiency |
| HEK293T | ABEmax | HEK | All | 289 | 5704 | Rep. 1 | 5,696 | 99.8% | 8 | 0.1% | 3 | 21.2 | 17.2 |
| | | site 2 | GFP | 290 | 3551 | Rep. 2 | 3,549 | 99.8% | 2 | 0.1% | 4 | 21.1 | 16.7 |
| | | (ABE | | 291 | 5172 | Rep. 3 | 5,168 | 99.9% | 4 | 0.1% | 3 | 20.6 | 16.7 |
| | | site 1) | | | | | | | | | | | |
| | miniABEmax- | HEK | All | 289 | 195 | Rep. 1 | 194 | 95.6% | 1 | 0.5% | 8 | 19.3 | 17.0 |
| | V82G | site 2 | GFP | 290 | 127 | Rep. 2 | 123 | 96.9% | 4 | 3.1% | 0 | 17.7 | 15.9 |
| | | (ABE | | 291 | 180 | Rep. 3 | 176 | 95.7% | 4 | 2.2% | 4 | 18.6 | 17.4 |
| | | site 1) | | | | | | | | | | | |
| | Target-AID | HEK | All | 289 | 11 | Rep. 1 | 5 | 20.0% | 6 | 24.0% | 14 | 14.9 | 9.5 |
| | | site 2 | GFP | 290 | 16 | Rep. 2 | 10 | 37.0% | 6 | 22.2% | 11 | 14.8 | 13.1 |
| | | (ABE | | 291 | 24 | Rep. 3 | 13 | 23.2% | 11 | 19.6% | 32 | 22.0 | 16.0 |
| | | site 1) | | | | | | | | | | | |
| | SPACE | HEK | All | 289 | 41 | Rep. 1 | 37 | 82.2% | 4 | 8.9% | 4 | 17.6 | 16.7 |
| | | site 2 | GFP | 290 | 29 | Rep. 2 | 26 | 81.3% | 3 | 9.4% | 3 | 16.6 | 15.4 |
| | | (ABE | | 291 | 23 | Rep. 3 | 20 | 76.9% | 3 | 11.5% | 3 | 18.3 | 18.5 |
| | | site 1) | | | | | | | | | | | |
| HEK293T | ABEmax | RNF2 | All | 292 | 3207 | Rep. 1 | 3,201 | 99.7% | 6 | 0.2% | 4 | 21.4 | 16.7 |
| | | site 1 | GFP | 293 | 3113 | Rep. 2 | 3,105 | 99.6% | 8 | 0.3% | 3 | 21.5 | 16.7 |
| | | | | 294 | 3997 | Rep. 3 | 3,993 | 99.7% | 4 | 0.1% | 10 | 22.0 | 17.3 |
| | miniABEmax- | RNF2 | All | 292 | 165 | Rep. 1 | 160 | 89.9% | 5 | 2.8% | 13 | 18.8 | 16.7 |
| | V82G | site 1 | GFP | 293 | 77 | Rep. 2 | 73 | 89.0% | 4 | 4.9% | 5 | 19.4 | 17.6 |
| | | | | 294 | 118 | Rep. 3 | 112 | 92.6% | 6 | 5.0% | 3 | 18.3 | 16.7 |
| | Target-AID | RNF2 | All | 292 | 25 | Rep. 1 | 17 | 38.6% | 8 | 18.2% | 19 | 19.8 | 15.0 |
| | | site 1 | GFP | 293 | 18 | Rep. 2 | 10 | 31.3% | 8 | 25.0% | 14 | 21.6 | 13.3 |
| | | | | 294 | 19 | Rep. 3 | 13 | 37.1% | 6 | 17.1% | 16 | 19.7 | 13.6 |
| | SPACE | RNF2 | All | 292 | 4 | Rep. 1 | 4 | 66.7% | 0 | 0.0% | 2 | 20.9 | 19.8 |
| | | site 1 | GFP | 293 | 26 | Rep. 2 | 22 | 68.8% | 4 | 12.5% | 6 | 19.7 | 17.1 |
| | | | | 294 | 25 | Rep. 3 | 21 | 65.6% | 4 | 12.5% | 7 | 17.4 | 16.7 |
| HEK293T | GFP only | — | All | 289 | 10 | Rep. 1 | 9 | 75.0% | 1 | 8.3% | 2 | 19.2 | 19.7 |
| | | | GFP | 290 | 10 | Rep. 2 | 7 | 63.6% | 3 | 27.3% | 1 | 18.5 | 17.3 |
| | | | | 291 | 13 | Rep. 3 | 12 | 85.7% | 1 | 7.1% | 1 | 18.6 | 16.7 |

### EXAMPLES

The invention is further described in the following examples, which do not limit the scope of the invention described in the claims.

### Methods

### Molecular Cloning

Constructs were cloned into the CMV from ABEmax-P2A-EGFP-NLS (Agel/Notl digest; Addgene #112101) or into the CAG backbone from SQT817 (Agel/Notl/EcoRV digest; Addgene #53373). All constructs with P2A-EGFP were cloned using either P2A-EGFP-NLS from ABEmax-P2A-EGFP-NLS or P2A-EGFP from BPK4335 serving as the template. SPACE was cloned using Gibson assembly with bpNLS-TadA7.10 (V82G)-SpCas9 (D10A)-bpNLS from miniABEmax-V82G (Addgene #131313), pmCDA1 from Target-AID (Addgene #131300), and dual UGIs from BE4max (Addgene #112093). All guide RNA plasmids were cloned by ligation into the pUC19-based entry vector BPK1520 (Bsmbl digest; Addgene #65777). All plasmids were midi or maxi prepped with the Qiagen Midi/Maxi Plus kits.

### Guide RNAs

All gRNAs were of the form 5′-NNNNNNNNNNNNNNNNNNNNCGTTTTAGAGCTAGAAATAGCAAGTTAAAATA AGGCTAGTCCGTTATCAACTTGAAAAAGTGGCACCGAGTCGGTGCTTTTTTT-3′. (SEQ ID NO: 169)

TABLE R
Shown below are the protospacer regions
(NNNNNNNNNNNNNNNNNNNN) for these gRNAs (all written 5′ to 3′).

| target gene/site | protospacer sequence | |
|---|---|---|
| ABE site 2 | GAGTATGAGGCATAGACTGC | SEQ ID NO: |
| | | 170 |
| ABE site 3 | GTCAAGAAAGCAGAGACTGC | SEQ ID NO: |
| | | 171 |
| ABE site 4 | GAGCAAAGAGAATAGACTGT | SEQ ID NO: |
| | | 172 |
| ABE site 7 | GAATACTAAGCATAGACTCC | SEQ ID NO: |
| | | 173 |
| ABE site 8 | GTAAACAAAGCATAGACTGA | SEQ ID NO: |
| | | 174 |
| ABE site 9 | GAAGACCAAGGATAGACTGC | SEQ ID NO: |
| | | 175 |
| ABE site 10 | GAACATAAAGAATAGAATGA | SEQ ID NO: |
| | | 176 |
| ABE site 11 | GGACAGGCAGCATAGACTGT | SEQ ID NO. |
| | | 177 |
| ABE site 14 | GGCTAAAGACCATAGACTGT | SEQ ID NO: |
| | | 178 |
| ABE site 15 | GTCTAGAAAGCTTAGACTGC | SEQ ID NO: |
| | | 179 |
| ABE site 17 | GACAAAGAGGAAGAGAGACG | SEQ ID NO: |
| | | 180 |
| ABE site 18 | ACACACACACTTAGAATCTG | SEQ ID NO: |
| | | 181 |
| ABE site 19 | CACACACACTTAGAATCTGT | SEQ ID NO: |
| | | 182 |
| EMX1 site 1 | GAGTCCGAGCAGAAGAAGAA | SEQ ID NO: |
| | | 183 |
| EMX1 site 2 | GTATTCACCTGAAAGTGTGC | SEQ ID NO: |
| | | 184 |
| FANCF site 1 | GGAATCCCTTCTGCAGCACC | SEQ ID NO: |
| | | 185 |
| HEK site 2 | GAACACAAAGCATAGACTGC | SEQ ID NO: |
| (ABE site 1) | | 186 |
| HEK site 3 | GGCCCAGACTGAGCACGTGA | SEQ ID NO: |
| | | 187 |
| HEK site 4 | GGCACTGCGGCTGGAGGTGG | SEQ ID NO: |
| | | 188 |
| PDCD1 site 1 | GCGTGACTTCCACATGAGCG | SEQ ID NO: |
| | | 189 |
| PPP1R12C site 2 | GGCACTCGGGGGCGAGAGGA | SEQ ID NO: |
| | | 190 |
| PPP1R12C site 3 | GAGCTCACTGAACGCTGGCA | SEQ ID NO: |
| | | 191 |
| PPP1R12C site 4 | GACCCTCAGCCGTGCTGCTC | SEQ ID NO: |
| | | 192 |
| PPP1R12C site 5 | GCTGACTCAGAGACCCTGAG | SEQ ID NO: |
| | | 193 |
| PPP1R12C site 6 | GGGGCTCAACATCGGAAGAG | SEQ ID NO: |
| | | 194 |
| PPP1R12C site 7 | GCTGGCTCAGGTTCAGGAGA | SEQ ID NO: |
| | | 195 |
| RNF2 site 1 | GTCATCTTAGTCATTACCTG | SEQ ID NO: |
| | | 196 |
| VEGFA site 4 | GAGGACGTGTGTGTCTGTGT | SEQ ID NO: |
| | | 197 |

### DNA Off-Targets Sites

TABLE S
Shown below are the sequences (NNNNNNNNNNNNNNNNNNNN) for
these off-target sites (all written 5' to 3').

| target site | sequence | |
|---|---|---|
| HEK site 2 off 1 | GAACACAATGCATAGATTGC | SEQ ID NO: |
| | | 198 |
| HEK site 2 off 2 | AAACATAAAGCATAGACTGC | SEQ ID NO: |
| | | 199 |
| HEK site 3 off 1 | CACCCAGACTGAGCACGTGC | SEQ ID NO: |
| | | 200 |
| HEK site 3 off 2 | GACACAGACTGGGCACGTGA | SEQ ID NO: |
| | | 201 |
| HEK site 3 off 3 | AGCTCAGACTGAGCAAGTGA | SEQ ID NO: |
| | | 202 |
| HEK site 3 off 4 | AGACCAGACTGAGCAAGAGA | SEQ ID NO: |
| | | 203 |
| HEK site 3 off 5 | GAGCCAGAATGAGCACGTGA | SEQ ID NO: |
| | | 204 |
| HEK site 4 off 1 | TGCACTGCGGCCGGAGGAGG | SEQ ID NO: |
| | | 205 |
| HEK site 4 off 2 | GGCTCTGCGGCTGGAGGGGG | SEQ ID NO: |
| | | 206 |
| HEK site 4 off 3 | GGCACGACGGCTGGAGGTGG | SEQ ID NO: |
| | | 207 |
| HEK site 4 off 4 | GGCATCACGGCTGGAGGTGG | SEQ ID NO: |
| | | 208 |
| HEK site 4 off 5 | GGCGCTGCGGCGGGAGGTGG | SEQ ID NO: |
| | | 209 |
| EMX1 site 1 off 1 | GAGTCTAAGCAGAAGAAGAA | SEQ ID NO: |
| | | 210 |
| EMX1 site 1 off 2 | GAGGCCGAGCAGAAGAAAGA | SEQ ID NO: |
| | | 211 |
| EMX1 site 1 off 3 | GAGTCCTAGCAGGAGAAGAA | SEQ ID NO: |
| | | 212 |
| EMX1 site 1 off 4 | GAGTCCGGGAAGGAGAAGAA | SEQ ID NO: |
| | | 213 |
| EMX1 site 1 off 5 | GAGCCGGAGCAGAAGAAGGA | SEQ ID NO: |
| | | 214 |
| FANCF site 1 off 1 | GGAACCCCGTCTGCAGCACC | SEQ ID NO: |
| | | 215 |
| FANCF site 1 off 2 | GGAGTCCCTCCTACAGCACC | SEQ ID NO: |
| | | 216 |
| FANCF site 1 off 3 | AGAGGCCCCTCTGCAGCACC | SEQ ID NO: |
| | | 217 |
| FANCF site 1 off 4 | ACCATCCCTCCTGCAGCACC | SEQ ID NO: |
| | | 218 |
| FANCF site 1 off 5 | GGATTGCCATCCGCAGCACC | SEQ ID NO: |
| | | 219 |
| FANCF site 1 off 6 | TGAATCCCATCTCCAGCACC | SEQ ID NO: |
| | | 220 |

### Cell Culture and Transfections

HEK293T cells (CRL-3216, ATCC) were grown in culture using Dulbeccos Modified Medium (Gibco) supplemented with 10% FBS (Gibco) and 1% penicillin-streptomycin solution (Gibco). Cells were passaged at ˜80% confluency every 2-3 days to maintain an actively growing population. Cells were passaged at ˜80% confluency every 4 days. Cells were used for experiments until passage 20, and were tested for mycoplasma every 4 weeks.
For DNA on-target experiments with 28 gRNAs, 1.25×10⁴ HEK293T cells were seeded into 96-well Flat Bottom cell culture plates (Corning), transfected 24 h post-seeding with 30 ng base editor or control, 10 ng gRNA, and 0.3 μL TransIT-X2 (Mirus), and harvested 72 h after transfection to obtain genomic DNA (gDNA). For DNA off-target experiments, 6.25×10⁴ HEK293T cells were seeded into 24-well cell culture plates (Corning), transfected 24 h post seeding with 150 ng base editor or control, 50 ng gRNA, and 1.5 μL TransIT-X2, and harvested 72 h after transfection to obtain gDNA. For RNA off-target experiments, 6.5×10⁶ HEK293T cells were seeded into 150 mm cell culture dishes (Corning), transfected 24 h post-seeding with 37.5 μg base editor or control, 12.5 μg gRNA, and 150 μL TransIT-293 (Mirus), and sorted 36-40 h after transfection. For co-expression of miniABEmax-V82G and Target-AID (ABE & CBE mix) vs SPACE experiments, 1.25×10⁴ HEK293T cells were seeded into 96-well cell culture plates, transfected 24 h post-seeding with 15 ng miniABEmax-V82G and 15 ng Target-AID for ABE & CBE mix, and 30 ng for both SPACE and the nCas9 control, 10 ng gRNA, and 0.3 μL TransIT-X2, and harvested 72 h after transfection to obtain gDNA. e.g.,

### FACS & RNA/DNA Harvest for RNA-Seq Experiments

Sorting of negative control and BE expressing cells as well as RNA/DNA harvest was carried out on the same day. Cells were sorted on a BD FACSARIAII 36-40h after transfection. We gated on the cell population on forward/sideward scatter after exclusion of doublets. We then sorted all GFP-positive cells and/or top 5% of cells with the highest FITC signal into pre-chilled 100% FBS and 5% of mean fluorescence intensity (MFI)-matched cells for nCas9-NLS negative controls, matching the MFI/GeoMean of top 5% of ABE or ABEmax-transfected cells. We used MFI-matching for these controls, as the bpNLS-32AAlinker-nCas9-bpNLS-P2A-EGFP (control) plasmid was smaller than ABEmax-P2A-EGFP—due to the lack of the TadA-TadA* heterodimer—and thus yielded higher transfection efficiency and overall higher FITC signal. After sorting, cells were spun down, lysed using DNA lysis buffer (Laird et al, 1991) with DTT and Proteinase K or RNA lysis buffer (Macherey-Nagel). gDNA was extracted using magnetic beads (made from FisherSci Sera-Mag SpeedBeads Carboxyl Magnetic Beads, hydrophobic according to Rohland & Reich, 2012), after overnight lysis. RNA then was extracted with Macherey-Nagel's NudeoSpin RNA Plus kit.

### High-Throughput Amplicon Sequencing & Base Editing Data Analysis

Genomic DNA was amplified using gene-specific DNA primers flanking desired target sequence. These primers included illumina-compatible adapter-flaps. The amplicons were molecularly indexed with NEBNext Dual Index Primers (NEB) or index primers with the same or similar sequence ordered from IDT. Samples were combined into libraries and sequenced on the Illumina MiSeq machine using the MiSeq Reagent Kit v2 or Micro Kit v2 (Illumina). Sequencing results were analyzed using a batch version of the software CRISPResso 2.0 (crispresso.rocks).

### RNA-Seq and Single Nucleotide Variant Calling

RNA library preparation was performed using Illumina's TruSeq Stranded Total RNA Gold Kit with initial input of ˜500 ng of extracted RNA per sample, using SuperScript 111 for first-strand synthesis (Thermo Fisher). rRNA depletion was confirmed during library preparation by fluorometric quantitation using the Qubit HS RNA kit before and after depletion (Thermo Fisher). For indexing, we used IDT-Illumina Unique Dual Indeces (Illumina). Libraries were pooled based on qPCR quantification (NEBNext Library Quant Kit for Illumina) and loaded onto a NextSeq (at MGH Cancer Center, PE 2x150, 500/550 MidOutput Cartridge) or HiSeq2500 in High Output mode (Broad Institute, PE 2x76). Illumina fastq sequencing reads were aligned to the human hg38 reference genome with STAR (Dobin et al., 2013, PMID: 23104886) and processed with GATK best practices (McKenna et al., 2010, PMID: 20644199: DePristo et al., 2011, PMID: 21478889). RNA variants were called using HaplotypeCaller, and empirical editing efficiencies were established on PCR-de-duplicated alignment data. Variant loci in ABE/ABEmax overexpression experiments were further required to have comparable read coverage in the corresponding control experiment (read coverage for SNV in control >90th percentile of read coverage across all SNVs in overexpression). Additionally, the above loci were required to have a consensus of at least 99% of reads calling the reference allele in control.

### Analysis of Potential Targets for the Correction or Generation of Multi-Nucleotide Variants (MNVs) by SPACE

A list of multi-nucleotide variants (MNVs) was obtained from Wang et. al. “Landscape of multi-nucleotide variants in 125,748 human exomes and 15,708 genomes” (pre-print at dx.doi.org/10.1101/573378; data file at storage.googleapis.com/gnomad-public/release/2.1/mnv/gnomad_mnv_coding.tsv). These were filtered to detect disease correcting or disease generating modifications enabled by SPACE, which are defined as annotated MNVs with a nearby PAM. Disease correcting conversions are defined as having targetable Cs and As in the ALT position with matching Ts and Gs in the REF position; whereas disease generating conversions are defined as the reverse scenario, with targetable Cs and As in the REF position with matching Ts and Gs in the ALT position. Patterns for selected disease correcting MNV codons include “GNT>ANC”, “GTN>ACN”, “NGT>NAC”, “NTG>NCA”, “TGN>CAN”, and “TNG>CNA”; whereas patterns for disease generating include “ACN>GTN”, “ANC>GNT”, “CAN>TGN”, “CNA>TNG”, “NAC>NGT”, and “NCA>NTG”. PAMs considered include NGG, NGA, and NG.

### Example 1. SPACE Induces Efficient Simultaneous C-to-T and A-to-G Editing in Human HEK293T Cells

Human HEK293T cells were transfected with plasmids encoding nCas9, miniABEmax-V82G, Target-AID and SPACE constructs (e.g., SEQ IDs 140-144; FIG. 1) and gRNAs targeting several genomic sites (e.g., SEQ ID 145-152). After 72 hours, gDNA was extracted and targeted amplicon sequencing was performed to determine the on-target DNA editing of SPACE constructs. On all genomic sites tested, efficient DNA base editing was detected by targeted deep sequencing on a MiSeq next-generation sequencing (NGS) device and both types expected substitutions, C-to-T as well as A-to-G were detected across the target sites tested, predominantly in positions 2-11 (FIGS. 2 and 3; with 1 being the most PAM-distal). Both types of edits were detected on a single allele in a substantial fraction of alleles, e.g., on HEK site 2 (FIG. 4). These data indicate that bifunctional adenine and cytosine base editing (BACE) can be performed using a single nCas9 fused to a dual-deaminase system. In this experiment, TadA*-V82G was fused to the N-terminus of nCas9 (D10A) and pmCDA1 was fused to the C-terminus, while 2 UGIs were fused C-terminal of pmCDA1. The constructs also contained two bipartite NLSs in order to enhance nuclear localization of the fusion proteins.

### Example 2. SPACE Editing Analysis, mRNA and RNP Production and Testing

Guide RNAs will be tested to determine how different sequence contexts might affect SPACE editing in two cell lines, as well as using SPACE mRNAs (produced via IVT or by TriLink) to electroporate primary human CD34+ and T cells. SPACE constructs will be subcloned into pET vectors with an N-terminal 6xHis-tag and codon-optimized for expression in E. coli to enable protein purification. RNPs will be electroporated with a Lonza device into HEK293T and primary human T cells.

### Example 3. SPACE Induces Reduced Indels and Higher Product Purity with Two Fused UGIs in Human Cells Compared to SPACEΔUGI

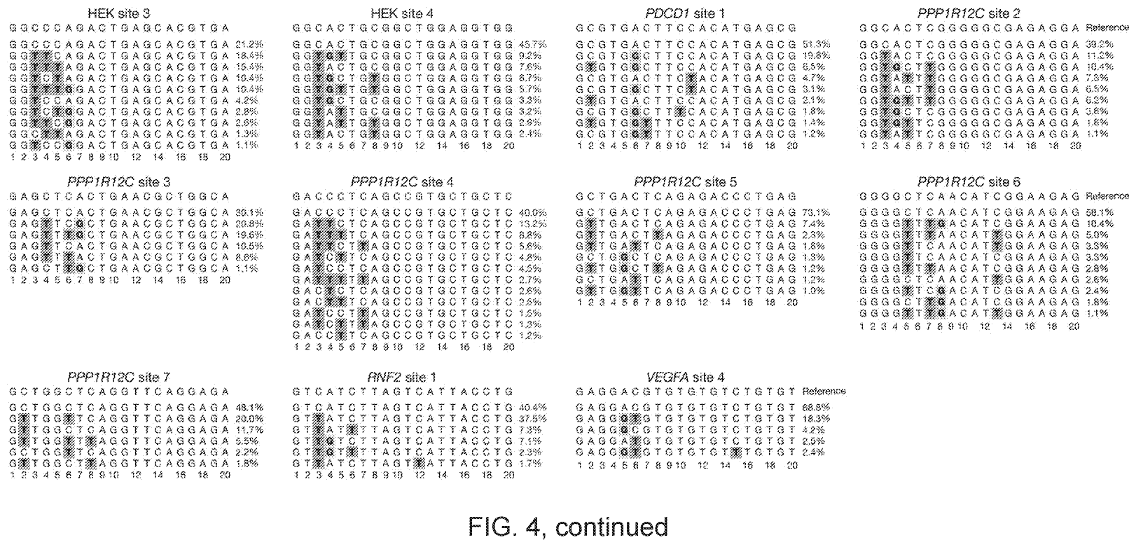

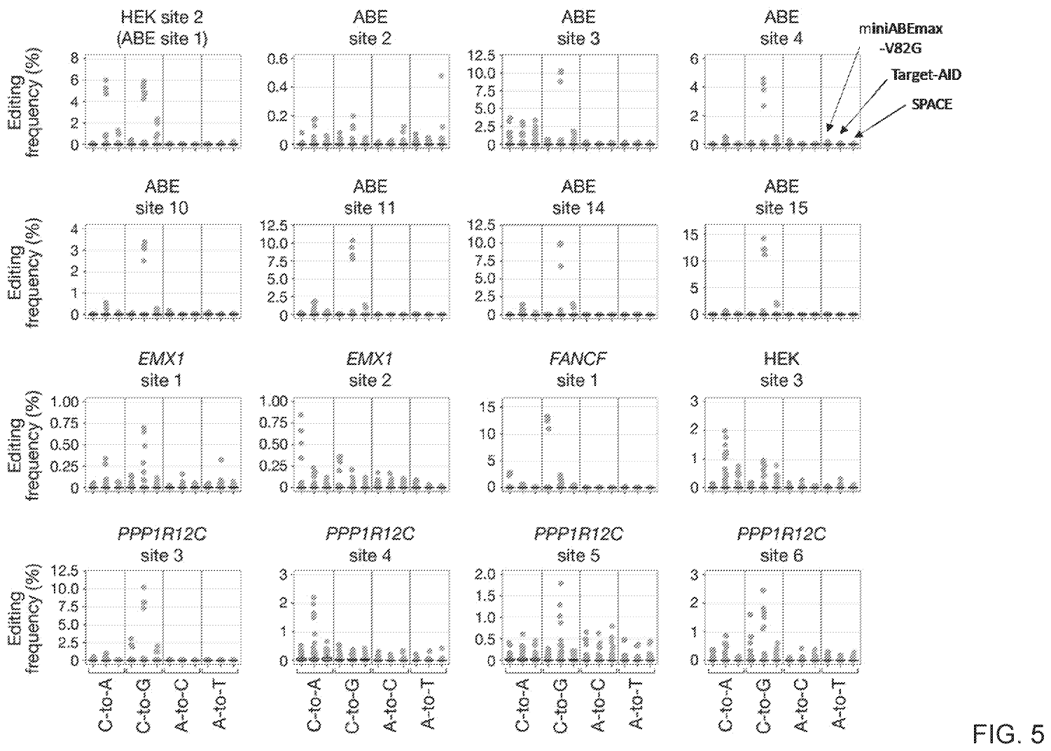

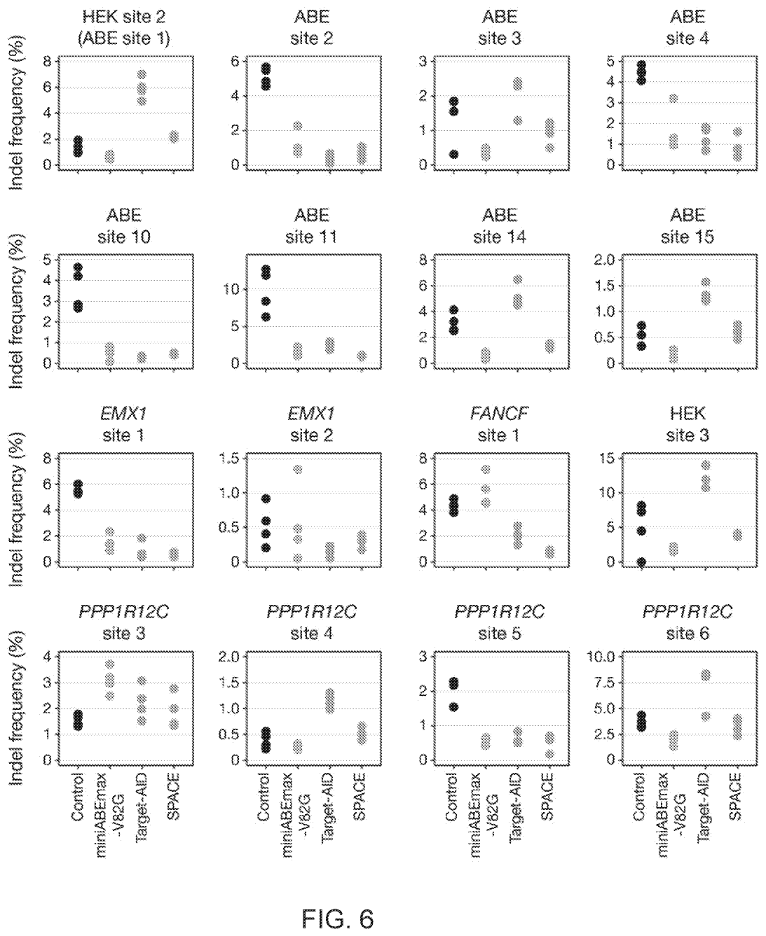

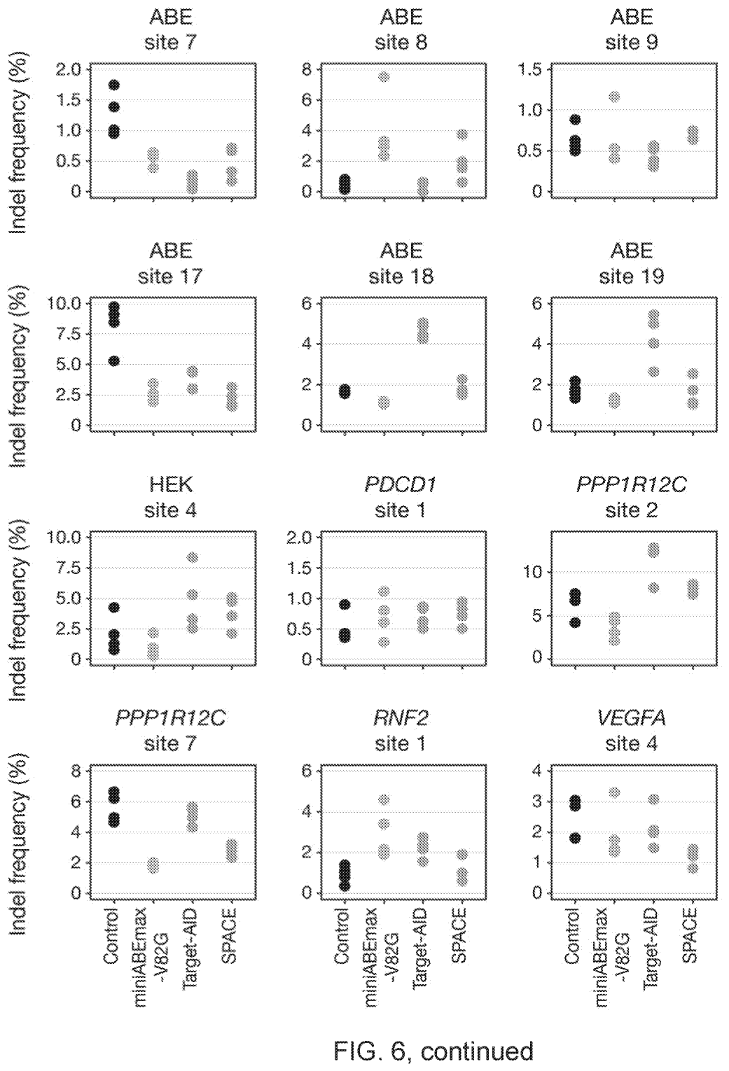

To determine if the UGIs play a vital role in maintaining product purity in the context of SPACE, i.e. enabling high C-to-T and A-to-G editing yield without C-to-N, A-to-N, or indel byproducts, human HEK293T cells were transfected with SPACEΔUGI. NGS results indicated that SPACE constructs—that contain two UGIs—showed substantially reduced base editing byproducts, e.g., markedly decreased rates of C-to-G edits and indels (FIGS. 2, 4, 5, and 6).

### Example 4. SPACE was Engineered Using Deaminase Components that Exhibit Reduced Off-Target RNA Editing

We have recently described RNA off-target editing induced by DNA base editors (Grunewald et al, Nature 2019). In order to reduce the potential RNA off-target editing of SPACE, we fused miniABEmax-V82G and pmCDA1, two deaminase domains with markedly reduced or undetectable RNA off-target editing respectively (FIGS. 1 and 11).

### Example 5. Evaluation of RNA Off-Target Editing Induced by SPACE

Unbiased detection of RNA off-target editing with the help of RNA-seq was assessed by transfecting cells with two different gRNAs and SPACE constructs that were co-translationally expressed with P2A-EGFP in 15 cm dishes and trypsinized 36 hours post-transfection. Subsequently, GFP+ cells were sorted on a BD FACSAria II and lysed to harvest both DNA and RNA. After efficient on-target editing was confirmed via targeted amplicon sequencing, RNA-seq was performed using a TruSeq stranded total RNA library prep and sequencing on a NextSeq 500 machine at the MGH.
To characterize the transcriptome-wide RNA off-target activity of SPACE, we performed RNA-seq from HEK293T cells co-expressing SPACE with a gRNA targeting HEK site 2 or RNF2 site 1. We also performed matched side-by-side RNA-seq experiments with HEK293T cells expressing miniABEmax-V82G, Target-AID, ABEmax (a positive control for RNA editing), and GFP (a negative control). Analysis of on-target DNA editing in the cells used for RNA-seq showed efficient editing with SPACE, miniABEmax-V82G, and Target-AID with both gRNAs. As expected, GFP negative control experiments showed very few RNA C-to-U edits (range of 1-3) and A-to-I edits (range of 7-12) while ABEmax induced relatively high numbers of A-to-I edits (range of 3,105-5,696), miniABEmax-V82G induced low numbers of A-to-I edits (range of 73-194), and Target-AID induced even lower numbers of C-to-U edits (range of 6-11). Cells expressing SPACE showed very few C-to-U edits (range of 0-4) and only small numbers of A-to-I edits (range of 4-37) edits. The generally lower numbers of RNA edits we observed in our current experiments relative to previously published studies are due to reduced sequencing depth we used here (˜14-18 million reads/sample) compared with our earlier work (^{˜}80-120 million reads/sample). Based on these results, we conclude that SPACE retains the reduced RNA-editing activities observed with miniABEmax-V82G and Target-AID, inducing very low numbers of unwanted RNA edits throughout the transcriptome (FIG. 11).

### Example 6. Evaluation of DNA Off-Target Editing Induced by SPACE

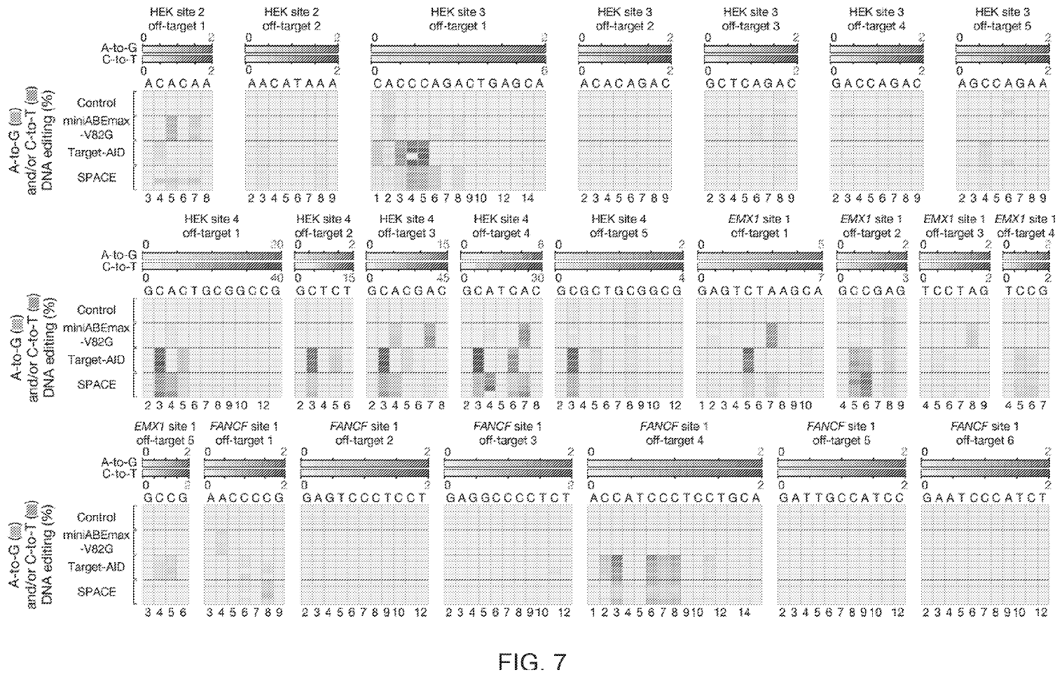

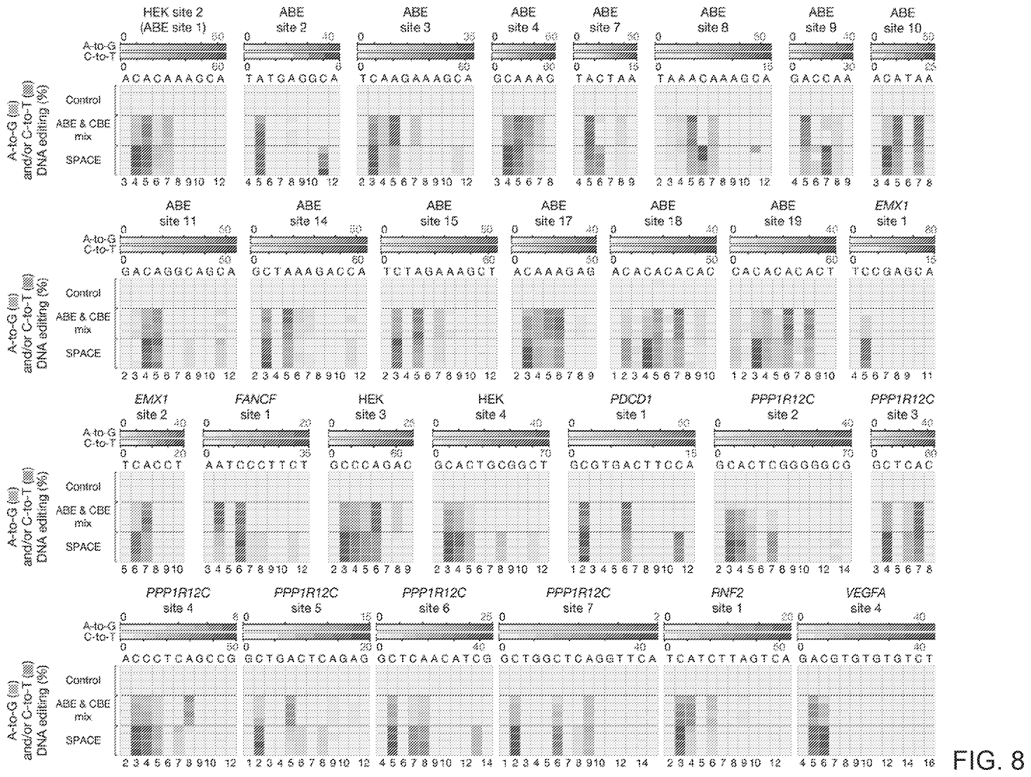

Cas9-dependent DNA off-target effects induced by SPACE were assessed by transfecting cells with HEK site 2, 3, and 4 as well as FANCF site 1 and EMX1 site 1 gRNAs. 23 genomic sites that have previously been described as known off-target sites for said gRNAs¹⁵ were sequenced with NGS to detect potential off-target base editing of SPACE constructs. Cas9-dependent DNA off-target effects observed with SPACE were comparable or lower relative to those observed with miniABEmax-V82G or Target-AID for 17 of these 23 off-target sites (FIG. 7).

### Example 7. SPACE Outperforms the Parallel Expression of Separate ABE & CBE Constructs

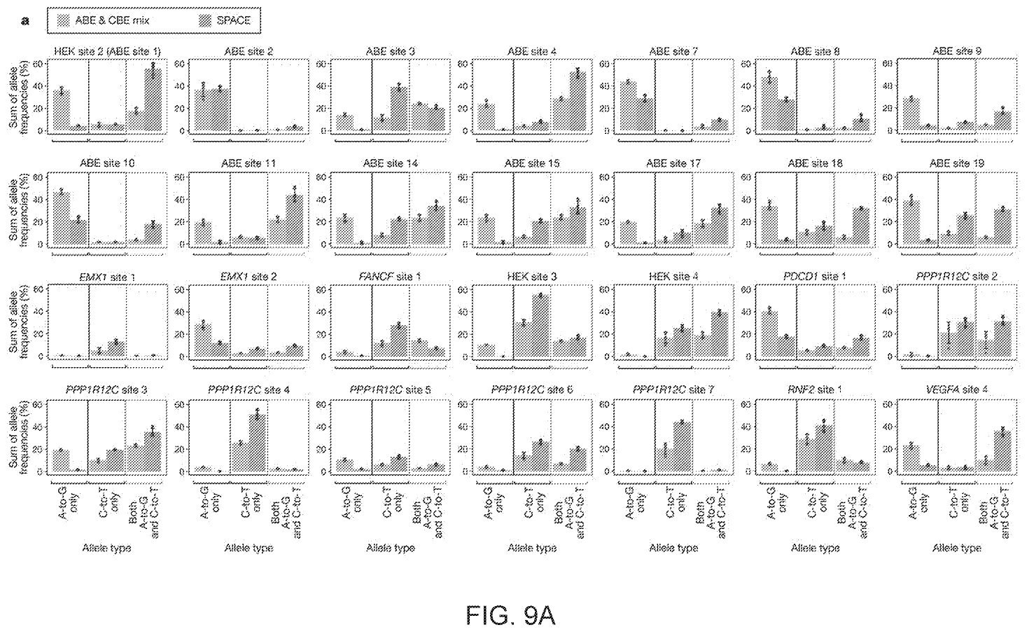

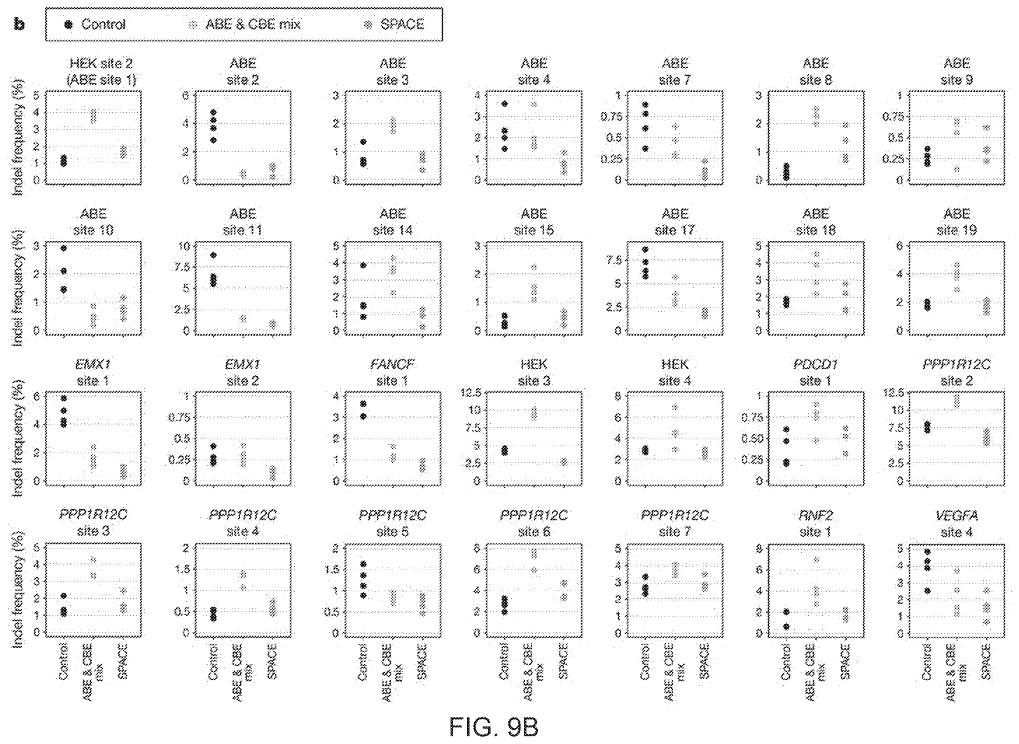

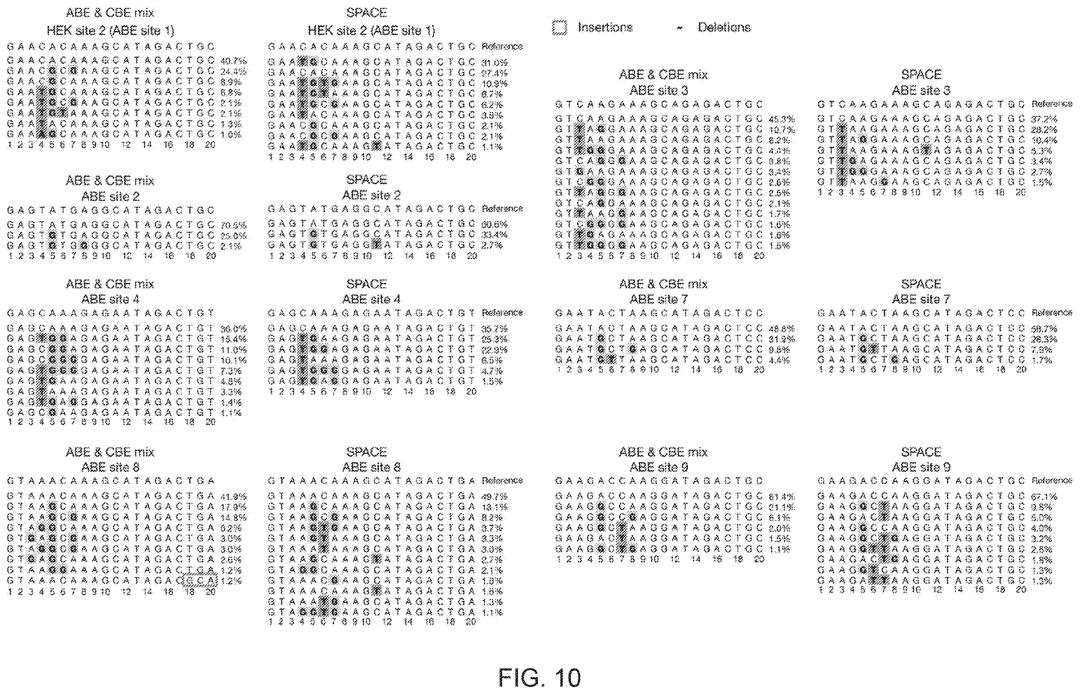

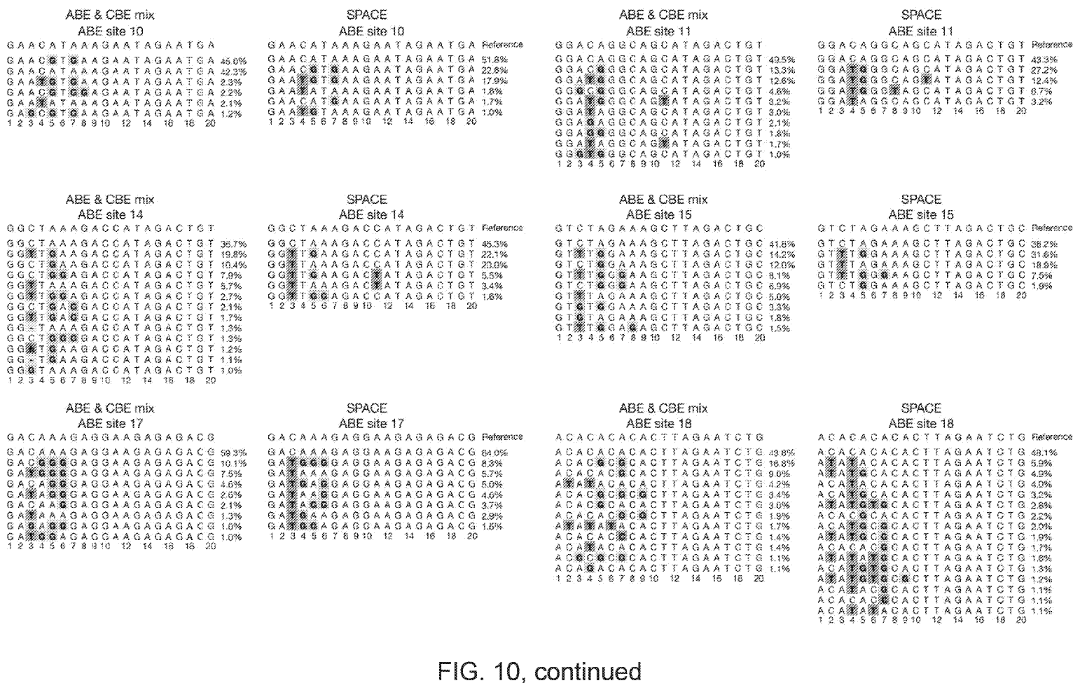

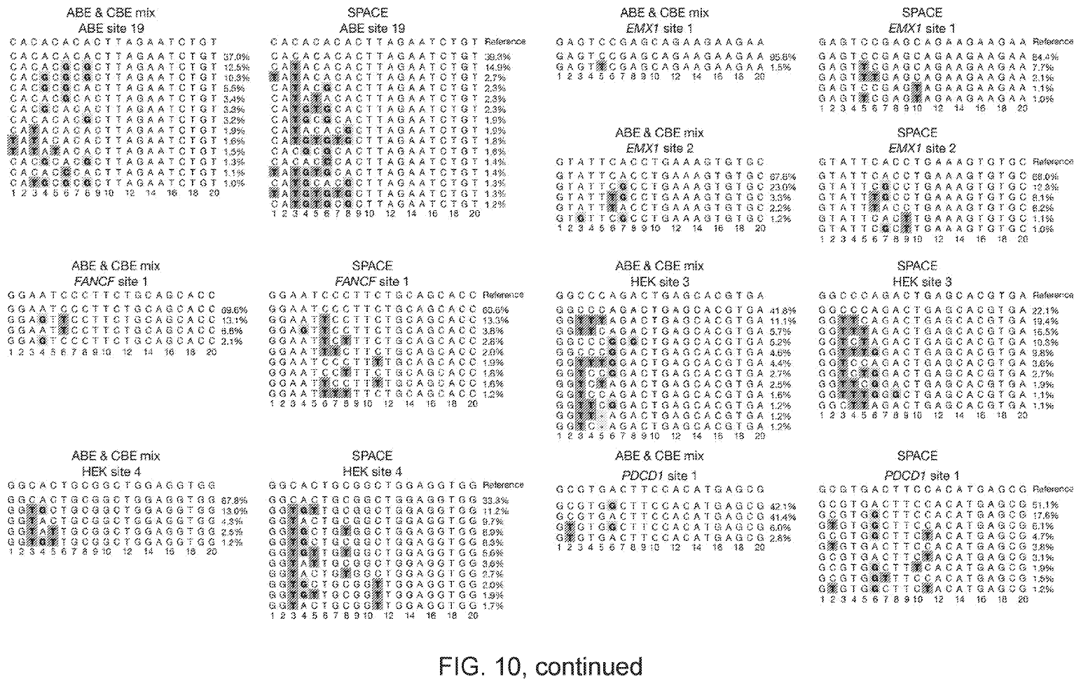

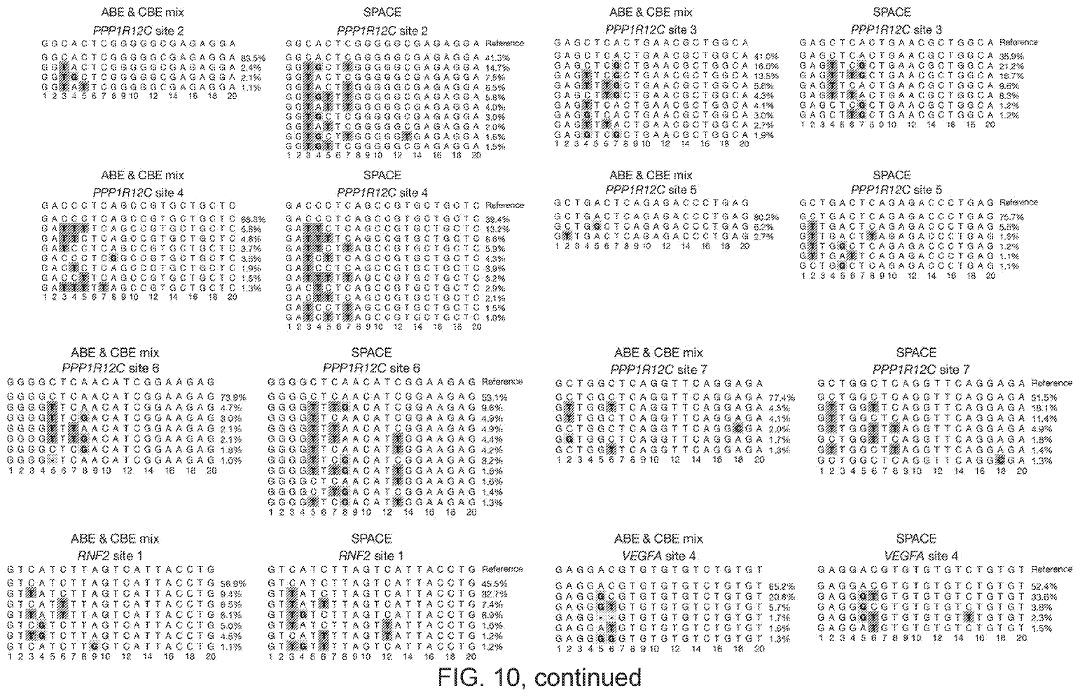

To test whether SPACE is more efficient at inducing dual edits than the combined effects of separate adenine and cytosine base editors, we also performed experiments in HEK293T cells in which we directly compared SPACE with co-expressed miniABEmax-V82G and Target-AID (“ABE & CBE mix”) for each of the 28 gRNAs. For 22 of the 28 gRNAs, the summed frequency of dual edited on-target alleles was higher with SPACE than with the “ABE & CBE mix”. Interestingly, the summed frequencies of on-target alleles harboring only A-to-G edits was higher with the “ABE & CBE mix” condition than with SPACE for 21 of these same 22 gRNAs whereas the summed frequencies of on-target alleles with only C-to-T edits was higher with SPACE than with the “ABE & CBE mix” for 16 of the 22 gRNAs. Unwanted indels induced by SPACE (range: 0.02-7.1%, mean: 1.44%) were lower or comparable to those observed with the “ABE & CBE mix” (range: 0.13-11.92%, mean: 2.88%) at 27 out of 28 sites tested. Although we cannot rule out that differences in the architecture of SPACE, miniABEmax-V82G, and Target-AID may affect their expression levels and/or activities, our results demonstrate that SPACE generally yields higher frequencies of dual-edited alleles and lower frequencies of indels at on-target sites compared to co-expression of standard editors harboring the same adenosine and cytidine deaminases individually (FIGS. 9 and 10).

### Example 8. New and/or Unique Codon and Amino Acid Modifications Enabled by SPACE

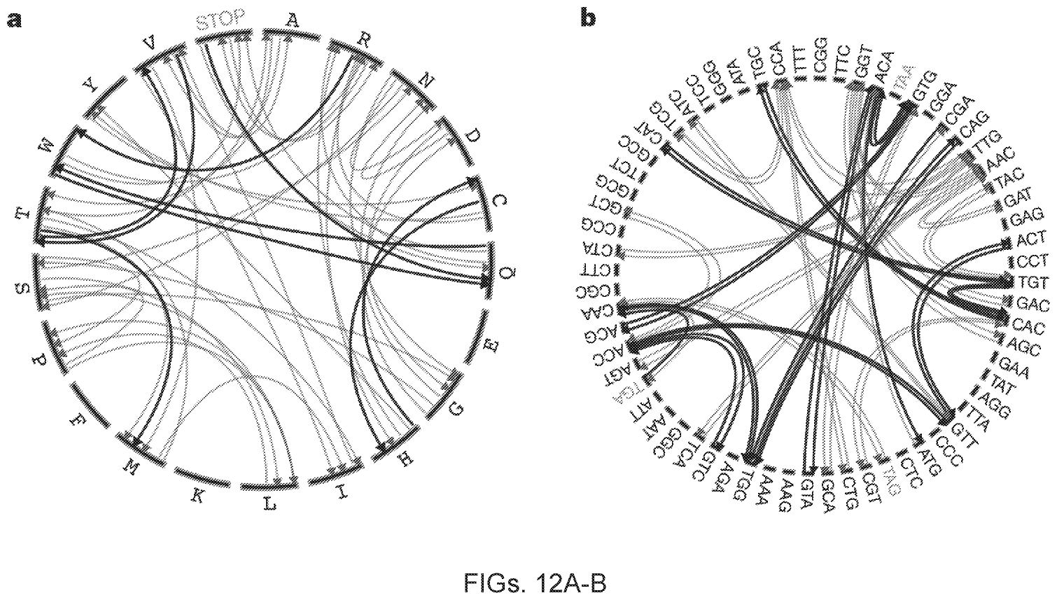

SPACE adds 60 additional codon changes (resulting in 18 amino acid substitutions) that cannot be created with existing single-action CBEs and ABEs (FIG. 12, Table D).

### Example 9. Using SPACE to Model and Correct Multi-Nucleotide Variants (MNVs) as Well as for Targeted Sequence Diversification

SPACE could be useful for creating or reverting multi-nucleotide variants (MNVs), a newly emerging category of sequence variants associated with disease (also see: https://www.biorxiv.org/content/10.1101/573378v1) (Tables E-M). Notably, among MNVs, TG-to-CA and CA-to-TG (both inducible by SPACE) are the most frequent consecutively arising adjacent dinucleotide MNVs (Kaplanis et al, Genome Res 2019). Furthermore, the greater combinatorial diversity of mutations that result with SPACE as compared with single-deaminase base editors could make it attractive for molecular recording systems (e.g., lineage tracing; McKenna et al, Science 2016) as well as for saturation mutagenesis screens, directed evolution, and protein engineering (Canver et al, Nature 2015; Hess et al, Nat Methods 2016).

### Example 10: Using SPACE to Induce Saturated Mutagenesis for Targeted Sequence Diversification

SPACE could be used for introducing A>G, T>C (A>G on the other strand), C>T, or G>A (C>T on the other strand) for every nucleotide available across a coding/non-coding region to generate a comprehensive library. This can enable high-throughput saturation mutagenesis screening and highly complex genotype-phenotype correlation to study a protein or gene of interest.

### REFERENCES

1. Komor, A. C., Kim, B., Packer, M. S., Zuris, J. A. & Liu, D. R. Programmable editing of a target base in genomic DNA without double-stranded DNA cleavage SUPPLEMENTS. Nature (2016). doi:10.1038/nature
2. Nishida, K. et al. Targeted nucleotide editing using hybrid prokaryotic and vertebrate adaptive immune systems. Science (80-). (2016). doi:10.1126/science.aaf8729
3. Gaudelli, N. M. et al. Programmable base editing of A•T to G•C in genomic DNA without DNA cleavage. Nature 1-27 (2017). doi:10.1038/nature24644
4. Rees, H. A. & Liu, D. R. Base editing: precision chemistry on the genome and transcriptome of living cells. Nat. Rev. Genet. (2018). doi:10.1038/s41576-018-0059-1
5. Grunewald, J. et al. Transcriptome-wide off-target RNA editing induced by CRISPR-guided DNA base editors. Nature (2019). doi:10.1038/s41586-019-1161-z
6. Zhou, C. et al. Off-target RNA mutation induced by DNA base editing and its elimination by mutagenesis. Nature (2019). doi:10.1038/s41586-019-1314-0
7. Rees, H. A., Wilson, C., Doman, J. L. & Liu, D. R. Analysis and minimization of cellular RNA editing by DNA adenine base editors SUPPLEMENT. Sci. Adv. 5, (2019).
8. Koblan, L. W. et al. Improving cytidine and adenine base editors by expression optimization and ancestral reconstruction. Nat. Biotechnol. (2018). doi:10.1038/nbt.4172
9. Thuronyi, B. W. et al. Continuous evolution of base editors with expanded target compatibility and improved activity. Nat. Biotechnol. (2019). doi:10.1038/s41587-019-0193-0
10. Komor, A. C. et al. Improved base excision repair inhibition and bacteriophage Mu Gam protein yields C: G-to-T: A base editors with higher efficiency and product purity. Sci. Adv. 1-10 (2017).
11. Kim, Y. B. et al. Increasing the genome-targeting scope and precision of base editing with engineered Cas9-cytidine deaminase fusions. Nat. Biotechnol. (2017). doi:10.1038/nbt.3803
12. Gehrke, J. M. et al. An APOBEC3A-Cas9 base editor with minimized bystander and off-target activities. Nat. Biotechnol. (2018). doi:10.1038/nbt.4199
13. Wang, X. et al. Efficient base editing in methylated regions with a human APOBEC3A-Cas9 fusion. Nat. Biotechnol. 36, (2018).
14. Grünewald, J. et al. Transcriptome-wide off-target RNA editing induced by CRISPR-guided DNA base editors (AAP). Nature (2019). doi:10.1038/s41586-019-1161-z
15. Tsai, S. Q. et al. GUIDE-seq enables genome-wide profiling of off-target cleavage by CRISPR-Cas nucleases. 1-3 (2014). doi:10.1038/nbt.3117

EXEMPLARY SEQUENCES

SEQ ID NO: 1
>tr|G3U0R4|G3U0R4_LOXAF Apolipoprotein B mRNA editing enzyme catalytic
subunit 1 OS = Loxodonta africana = African elephant OX = 9785 GN = APOBEC1
PE = 4 SV = 1
FRRRIKPWEFEIFFDPRQLRKETCLLYEIMIGTSHKVWRNSGQNTTKHVEVNFI
EKFTSERKLCPSISCSITWFLSWSPCVVECSKAIREFLRQHPNVTLVIYVARLFHH
MDQRNRQGLKDLILSGITVQIMRVSEYHHCWRNFVSYSPGEETYWPRYPPLW
MMMYALELHCIILSLPPCLKISRRCQHQLTLFSLTPQKCHYQMIPPYILLATGLIEP
PMTWR
SEQ ID NO: 2
>tr|A0A0M3N0G8|A0A0M3N0G8_PROAN APOBEC-1 OS = Protopterus
annectens OX = 7888 PE = 2 SV = 1
MVQKRTSASKTRMTKKVLLSEYQKFYYSPRTCIGYVIQYDEDNVIFQNWICNKR
TTHAELQCIYEIKQNSLIKRFTPCTLKWYMSWTPCSECANEIIRFLNKFCQVKLEI
CAARIYFHKKKDNRRALRNLVKAGVKLTTMRWKDYKSMWRRFGTGEEIKKYEF
FEKSSDHKSVNWRVVTLKKILKEKDRDSDLENALSLLKI
SEQ ID NO: 3
>tr|A0A151P6M4|A0A151P6M4_ALLMI C->U-editing enzyme APOBEC-1
OS = Alligator mississippiensis OX = 8496 GN = APOBEC1A PE = 4 SV = 1
MAVEEEKGLLGTSQGWKIELKDFQENYMPSTWPKVTHLLYEIRWGKGSKVWR
NWCSNTLTQHAEVNCLENAFGKLQFNPPVPCHITWFLSWSPCCQCCRRILQFL
RAHSHITLVIKAAQLFKHMDERNRQGLRDLVQSGVHVQVMDLPDYRYCWRTFV
SHPHEGEGDFWPWFFPLWITFYTLELQHILLQQHALSYNL
SEQ ID NO: 4
>tr|F1CGT0|F1CGT0_ANOCA Apolipoprotein B mRNA-editing enzyme 1a
isoform (Fragment) OS = Anolis carolinensis OX = 28377 PE = 2 SV = 1
KAAILLSNLFFRWQMEPEAFQRNFDPREFPECTLLLYEIHWDNNTSRNWCTNK
PGLHAEENFLQIFNEKIDIKQDTPCSITWFLSWSPCYPCSQAIIKFLEAHPNVSLEI
KAARLYMHQIDCNKEGLRNLGRNRVSIMNLPDYRHCWITFVVPRGANEDYWP
QDFLPAITNYSRELDSILQD
SEQ ID NO: 5
>tr|A0A091EQ78|A0A091EQ78_CORBR C->U-editing enzyme APOBEC-1
(Fragment) OS = Corvus brachyrhynchos OX = 85066 GN = N302_10757 PE = 4
SV = 1
RWKIEPGDFQINYSPSQHRRGVYLLYEIRWRRGSIWRNWCSNTHRQHAEVNF
LENCFKDRPQVPCSITWFLSASPCGKCSKRILEFLKSRPYVTLKIYAAKLFRHHD
IRNREGLCNLGMHGVTIHIMNLEDYSYCWRNFVVY
SEQ ID NO: 6
>tr|A0A091IIG0|A0A091IIG0_CALAN C->U-editing enzyme APOBEC-1
(Fragment) OS = Calypte anna OX = 9244 GN = N300_12023 PE = 4 SV = 1
RWKIQPNDFKRNYQPGRRPNVVYLLYEIRWRRGTIWRNWCSNEFPQHAEDNF
FQNRFNAVPSVSCSITWFLSTTPCGRCSKRILEFLRLHPNVTLKIYAARLFRHLD
NRNRQGLRKLASNGVIIQIMGLPDYSYSWKKFVAY
SEQ ID NO: 7
>tr|A0A2U4ALA1|A0A2U4ALA1_TURTR C->U-editing enzyme APOBEC-1
isoform X1 OS = Tursiops truncatus OX = 9739 GN = APOBEC1 PE = 4 SV = 1
MIICWSTGPSAGDATLRRRIEPWEFEVSFDPRELSKETRLLYEIKWGKSQRIWR
HSGKNTTKHVERNFIEQITSERRFHRSVSCCIIWFLSWSPCWECSEAIREFLKQ
HPRVTLLIYVARLFQHMDPRNRQGLRDLTHSGVTIQIMGPTEYDYCWRYFVNY
APGKEAHWPRYPPLLMKLYALELHCIILGLPPCLNISRYQNQLTLFRPILRNCHY
QMIPPHILLHTGLIQLPLTWR
SEQ ID NO: 8
>tr|A0A093FY71|A0A093FY71_TYTAL C->U-editing enzyme APOBEC-1
(Fragment) OS = Tyto alba OX = 56313 GN = N341_11998 PE = 4 SV = 1
RWKIQPNDFKRNFLPGQHPKVVYLMYEIRWIRGTAWRSWCSNNSKQDAEVNL
LENCFKAMPSVFCSVTWVLFTTPCGKCFRRILEFLRVHSNVALERYAAQLFRHL
DICNWQGIRSLAMNGVIIHIMNLADYSYCWKRFVAY
SEQ ID NO: 9
>tr|L5KGJ8|L5KGJ8_PTEAL C->U-editing enzyme APOBEC-1 OS = Pteropus
alecto OX = 9402 GN = PAL_GLEAN10015600 PE = 4 SV = 1
MWVLFDILISWSTGPSTGDPTLRRRIEPWEFEVFFDPRELRKEACLLYEIQWGT
SHKIWRNSGKNTTKHVELNFIEKFTSERHFCSSVSCSIIWFLSWSPCWECSKAI
REFLSORPTVTLVIFVSRLFQHMDQQNRQGLRDLINSGVTIQIMRASEYDHCWR
NFVNYPPGKEAHWPRYPPLWIVIKLYALELHCIILSLPPCVMISRRCQKQLTLFTLI
LKKCHYQMIPAHILLATGLIQVPVTWR
SEQ ID NO: 10
>tr|A0A2K6KS69|A0A2K6KS69_RHIBE CMP/dCMP-type deaminase domain-
containing protein OS = Rhinopithecus bieti OX = 61621 GN = APOBEC1
PE = 4 SV = 1
MTSEKGPSTGDPURRRIEPWEFDIFYDPRELRKEACLLYEIKWGMSWKIWRS
SGKNTTNHVEVNFIEKFTSERRFHSSISCSITWFLSWSPCWDCSQAIRKFLSQH
PGVTLVIYVARLFWHTDQQNRQGLRDLVNSGVTIQMMTASEYYHCWRNFVNY
PPGEEAHWPRYPPLWMMLYALELHCIILSLPPCLKISRRWQNHLTFFRLHLQNC
HYQTIPPHILLATGLIQPSVTWR
SEQ ID NO: 11
>tr|A0A2Y9NGP5|A0A2Y9NGP5_DELLE C->U-editing enzyme APOBEC-1
isoform X1 OS = Delphinapterus leucas OX = 9749 GN = APOBEC1 PE = 4 SV = 1
MIICWSTGPSAGDATSRRRIEPWEFEVSFDPRELCKETRLLYEIKWGKSQHVW
RHSDKNTTKHVECKFIEKITSERHFHPSVSCCIIWFLSWSPCWECSKAIREFLNQ
HPRVTLFIYVARLFQHMDPQNRQGLRDLIHSGVTIHVMGPTEYDYCWRNFVNY
PPGKEAHWPRYPPMLMKLYALELHCIILGLPPCLNISRYQNQLTLFRLIPQNCHY
RMIPPHILLHRGLIRLPLTWR
SEQ ID NO: 12
>tr|A0A218ULD2|A0A218ULD2_9PASE C->U-editing enzyme APOBEC-1
OS = Lonchura striata domestica OX = 299123 GN = APOBEC1 PE = 4 SV = 1
MYRRKMRGMYISKRALRKHFDPRNYPRETYLLCELQWRGSHKSWQHWLRND
DSKDCHAEKYFLEEIFEPRSYNICDMTWYLSWSPCGECCDIIQDFLEEQPNVNI
NIRIARLYYADRASNRRGLMELANSPGVSIEIMDADDYNDCWETFIQPGVYYRF
SPENFESAIRRNCSQLEDILQGLHL
SEQ ID NO: 13
>tr|A0A0Q3WRD0|A0A0Q3WRD0_AMAAE C->U-editing enzyme APOBEC-1
OS = Amazona aesfiva OX = 12930 GN = AAES_27783 PE = 4 SV = 1
MLPAPAPVPLVLPLQGGGVVVVTVGVXPTALLQPSGAPEVARTFVGAVIAFVIA
EYVDTSVSEDTTICGMYIPKEALKYHFDPREVXRDTYLLCILRWGETGTPWSHW
VKNYRYHAEVYFLEKIFQTRKSSKNINCSITWYLSWSPCAKCCRKILNFLKKHSY
VSIKIHVARLFRIDDKETXQNLKNLGSLVGVTVSVMEXEDYTNCWKTFIRGHAD
GDSWIDDLKSEIRKNRLKFQGIFKDLPHQTEDVDFWLILAANPGPAWFSFSGYT
GWAVASKAPSLLSPLSCLTRLLTP
SEQ ID NO: 14
>tr|A0A2K6U925|A0A2K6U925_SAIBB Apolipoprotein B mRNA editing enzyme
catalytic subunit 1 OS = Saimiri boliviensis boliviensis OX = 39432
GN = APOBEC1 PE = 4 SV = 1
MTSERRRIEPWEFSISYDPRELCKETCLLYEIKWGMSWKIWRSSGKNTTNHVE
VNFIEKFTSERHFHSSVSCSITWFLSWSPCWECSQAIREFLSQHPGVTLVIYVA
RLFQHMDQQNRQGLRELVNSGVTIQIMTASEYYHCWRNFVNYPPGEEAHWPR
HPPLWMMLYALELHCIIL
SEQ ID NO: 15
>tr|A0A2R9A0R0|A0A2R9A0R0_PANPA CMP/dCMP-type deaminase domain-
containing protein OS = Pan paniscus OX = 9597 GN = APOBEC1 PE = 4 SV = 1
ISWSTGPSTGDPTLRRRIEPWEFDVFYDPRELRKEACLLYEIKWGMSRKIWRSS
ERDFHPSISCSIPATLSWSPCWECSQAIREFLSQHPGVTLVIYVARLFWHMDQ
QNRQGLRDLVNSGVTIQIMTASEYYHCWRNFVNYPPGDEAHWPQYPPLWMM
LYALELHCIILSLPPCLKISRRWQNHLTFFSLHLQNCHYQTIPPHILLATGLIHPSV
AWR
SEQ ID NO: 16
>sp|Q694B3|ABEC1_PONPY C->U-editing enzyme APOBEC-1 OS = Pongo
pygmaeus OX = 9600 GN = APOBEC1 PE = 3 SV = 2
MTSEKGPSTGDPTLRRRIESWEFDVFYDPRELRKETCLLYEIKWGMSRKIWRS
SGKNTTNHVEVNFIKKFTSERRFHSSISCSITWFLSWSPCWECSQAIREFLSQH
PGVTLVIYVARLFWHMDQRNRQGLRDLVNSGVTIQIMRASEYYHCWRNFVNYP
PGDEAHWPQYPPLWMMLYALELHCIILSLPPCLKISRRWQNHLAFFRLHLQNC
HYQTIPPHILLATGLIHPSVTWR
SEQ ID NO: 17
>tr|E1BP99|E1BP99_BOVIN Apolipoprotein B mRNA editing enzyme catalytic
subunit 1 OS = Bos taurus OX = 9913 GN = APOBEC1 PE = 4 SV = 1
MASDRGPPAGDPTLRRRIEPWEFEFSFDPRKFCKEACLLYEIQWGNNRDVWR
HSGKNTTKHVERNFIEKIASERYFCPSIRCFIFWYLSWSPCWECSKAIREFLNQH
PNVTLVIYIARLFQHMDPQNRQGLKDLVQSGVTIQVMRAPEYEYCWRNFVNYP
RGKEAHWPRYPPLWMNLYALELYCIILGLPPCLHISRRYQNQLIVFRLTLQNCHY
QMIPPYILLATGMVQLPMTWR
SEQ ID NO: 18
>tr|S7PYX0|S7PYX0_MYOBR C->U-editing enzyme APOBEC-1 OS = Myotis
brandtii OX = 109478 GN = D623_10002956 PE = 4 SV = 1
MDEQNRQGLRDLIKSGVTVQIMTTPEYDYCWRNFVNYPPGKDTHCPMYPPLW
MKLYALELHCIILSLPPCLMISRRCQKQLTWYRLNLQNCHYQQIPHHILLATVWI
SEQ ID NO: 19
>tr|M3WB96|M3WB96_FELCA Apolipoprotein B mRNA editing enzyme catalytic
subunit 1 OS = Felis catus OX = 9685 GN = APOBEC1 PE = 4 SV = 2
MASDKGPSAGDATLRRRIEPREFEVFFDPRELRKEACLLYEIKWGTSHRIWRNS
GRNTANHVELNFIEKFTSERHFCPSVSCSITWFLSWSPCWECSKAIRGFLSQHP
SVTLVIYVSRLFWHLDQQNRQGLRDLVNSGVTVQIMRVPEYDHCWRNFVNYP
PGEEDHWPRYPVVWMKLYALELHCIILSLPPCLKILRRCQNQLTLFRLTLQNCH
YQMIPPHILLATGLIQLPVTWR
SEQ ID NO: 20
>tr|A0A2K5PZC0|A0A2K5PZC0_CEBCA CMP/dCMP-type deaminase domain
containing protein OS = Cebus capucinus imitator OX = 1737458 GN = APOBEC1
PE = 4 SV = 1
MTSERGPSTGDPTLRRRIEPWEFYISYDPKELCKETCLLYEIKWGMSWKIWRS
SGKNTTNHVEVNFIEKFTSERRFHSSISCSITWFLSWSPCWECSQAIREFLSQH
PGVTLVIYVARLFQHMDQQNROGLRDLVNSGVTIQIMRASEYYYCWRNFVNYP
PGEEAHWPRHPPLWMMLYALELHCIILGLPPCLKISRRRQNRLTFFRLHLQNCH
YQMIPPHILLAAGLIQPSVTWR
SEQ ID NO: 21
>tr|H2Q5C6|H2Q5C6_PANTR Apolipoprotein B mRNA editing enzyme catalytic
subunit 1 OS = Pan troglodytes OX = 9598 GN = APOBEC1 PE = 4 SV = 1
MTSEKGPSTGDPTLRRRIEPWEFDVFYDPRELRKEACLLYEIKWGMSRKIWRS
SGKNTTNHVEVNFIKKFTSERHFHPSISCSITWFLSWSPCWECSQAIREFLSQH
PGVTLVIYVARLFWHMDQQNRQGLRDLVNSGVTIQIMRASEYYHCWRNFVNYP
PGDEAHWPQYPPLWMMLYAL
ELHCIILSLPPCLKISRRWQNHLTFFSLHLONCHYQTIPPHILLATGLIHPSVAWR
SEQ ID NO: 22
>tr|A0A1U7S7K7|A0A1U757K7_ALLSI C->U-editing enzyme APOBEC-1-like
OS = Alligator sinensis OX = 38654 GN = LOC102373005 PE = 4 SV = 1
MGEHWQYAGSGEYIPQDQFEENFDPSVLLAETHLLSELTWGGRPYKHWYENT
EHCHAEIHFLENFSSKNRSCTITWYLSWSPCAECSARIADFMQENTNVKLNIHV
ARLYLHDDEHTROGLRYLMKMKRVTIQVMTIPDYTYCWNTFLEDDGEDESDDY
GGYAGVHEDEDESDDDDYLPTHFAPWIMLYSLELSCILQGFAPCLKIIQGNHMS
PTFQLHVQDQEQKRLLEPANPWGAD
SEQ ID NO: 23
>tr|G31IS7|G3I1S7_CRIGR C->U-editing enzyme APOBEC-1 OS = Cricetulus
griseus OX = 10029 GN = I79_017346 PE = 4 SV = 1
MTEQEYCYCWRNFVNYPPSNEVYWPRYPNVWMRMYALELYCIVLGLPPCLKII
RRHQHPLTFFTLHLQSCHYQRIPPHILWATGLV
SEQ ID NO: 24
>tr|A0A094MFH1|A0A094MFH1_ANTCR C->U-editing enzyme APOBEC-1
(Fragment) OS = Antrostomus carolinensis OX = 279965 GN = N321_09417
PE = 4 SV = 1
RWKMQPNDFKRNYLPVQYPNMVYLLYEIRWSTGTIWRNWCSNNSTQHAEVNF
LENRFNSRPSVSCSITWVLSTTPCGKCSTKILEFLRLHPNVTLKIYAAKLFKHLDI
RNRQGLRNLAMNGVIIRIMNLADYSYCWKTFVAY
SEQ ID NO: 25
>tr|A0A2K6EVT9|A0A2K6EVT9_PROCO CMP/dCMP-type deaminase domain-
containing protein OS = Propithecus coquereli OX = 379532 GN = APOBEC1
PE = 4 SV = 1
MTSEKRRIEPWEFEAFFDPRELRKEACLLYEIKWGASHKIWRNTGKSTTRHVEV
NFIEKFTSERRSDSLISCSITWFLSWSPCWECSKAIREFLSQHPNVTLVIYVARLF
WHMNQQNRQGLRDLINSGVTVQIMGVSEYCHCWRNFVNYPPGKEASCPTYP
PLWMTLYALELHCIILSLPPCLKISRRCQNQLTFFRLTPQN0HYQTIPPHILLATG
LIQPSVPAIR
SEQ ID NO: 26
>tr|G8F4P7|G8F4P7_MACFA C->U-editing enzyme APOBE0-1 (Fragment)
OS = Macaca fascicularis OX = 9541 GN = EGM_20518 PE = 4 SV = 1
GPSTGDPTLRRRIEPWEFDIFYDPRELRKEACLLYEIKWGMSPKIWRSSGKNTT
NHVEVNFIEKLTSERRFHSSISCSITWFLSWSPCWECSQAIREFLSQHPGVTLVI
YVARLFWHTDQQNRQGLRDLVNSGVTIQIMRASEYYHCWRNFVNYPPGEEAH
WPRYPPLWMMLYALELHCIILSLPPCLKISRRWQNHLTFFRLHLQNCHYQMIPP
HILLATGLIQPSVTWR
SEQ ID NO: 27
>tr|A0A091V7F8|A0A091V7F8_NIPNI C->U-editing enzyme APOBEC-1
(Fragment) OS = Nipponia nippon OX = 128390 GN = Y956_13652 PE = 4
SV = 1
RWKIQPNDFRSNYLPCQHPRVVYLLYEIRWSRGTIWRNWCSNNSTQHAEVNF
LENCFKAMPSVPCSITWVLSTTPCGKCSRRILEFLRVHPNVTLEIYAAKLFKHLDI
RNRQGLRNLAKNGVVIRIMKLADYSYWWKRFVAY
SEQ ID NO: 28
>tr|A0A091SSF0|A0A091SSF0_PELCR C->U-editing enzyme APOBEC-1
(Fragment) OS = Pelecanus crispus OX = 36300 GN = N334_11718
PE = 4 SV = 1
RWKLQPEDFKRNYLPGQHPKVVYLLYEIRWSRGTIWRSWCSNNSKQHAEVNF
LENCFKARPSVSCSITWVLSTTPCGKCSRRILEFLRVHPNVTLEIYAAKLFKHLDI
RNQQGLRNLAWINGVIIRIMNLADYSYCWKRFVAH
SEQ ID NO: 29
>tr|A0A091CVE5|A0A091CVE5_FUKDA C->U-editing enzyme APOBEC-1
OS = Fukomys damarensis OX = 885580 GN = H920_16562 PE = 4 SV = 1
MSDPEFCHCWRNFVNYPPGQEARWPRFPPVWTMLYTLELCCVLLNLPPCLKI
SRRCHNQLAFFQLNLQNCHYRAIPPAVLFAVGLIHPFVAWA
SEQ ID NO: 30
>tr|L5LUG3|L5LUG3_MYODS C->U-editing enzyme APOBEC-1 OS = Myotis
davidii OX = 225400 GN = MDA_GLEAN10003736 PE = 4 SV = 1
MASDAGKMDRGPVSFIVLKSVETLCVRRIEPWEFEAIFDPRELRKEACLLYEIK
WGTGHKIWRHSGKNTTRHVEVNFIEKITSERQFCSSTSCSIIWFLSWSPCWEC
SKAITEFLRQRPGVTLVIYVARLYHHIVIDEQNRQGLRDLVKSGVTVQIMTTPEYD
YCWRNFVNYPPGKDTHCPIYPPLLIVIKLYALELHCIILSLPPCLMISRRCQKQLTW
YRLNLQNCHYQQIPHHILLATAWI
SEQ ID NO: 31
>tr|F1PUJ5|F1PUJ5_CANLF Apolipoprotein B mRNA editing enzyme catalytic
subunit 1 OS = Canis lupus familiaris OX = 9615 GN = APOBEC1 PE = 4 SV = 2
MASDKGPSAGDATLRRRIEPWEFEGFFDPRELRKETCLLYEIQWGTSHKTWRN
SGKNTTNHVEINFMEKFAAERQYCPSIRCSITWFLSWSPCWECSNAIRGFLSQ
HPSVTLVIYVARLFWHTDPQNRQGLRDLINSGVTIQIMTVPEYDHCWRNFVNYP
PGKEDHWPRYPVLWMKLYALELHCIILNLPPCLKISRRNQHQLTLFRLTLQDCH
YQTIPPPILLDMGLIQPLVTWR
SEQ ID NO: 32
>tr|A0A093GVH6|A0A093GVH6_DRYPU C->U-editing enzyme APOBEC-1
(Fragment) OS = Dryobates pubescens OX = 118200 GN = N307_04563 PE = 4
SV = 1
RWKIHPDEFKLNYVPVGRPRVVYLLYEIRWSRGSIWRNWCSNSSTQHAEVNFL
ENCFKAMPSVSCSITWFLSTTPCGNCSRRILEFLRAHPKVTLAIHAAKLFKHLDV
RNRHGLKALATDGVVLHIMSIADYRYCWTKFVAY
SEQ ID NO: 33
>tr|A0A2K5Z8Y4|A0A2K5Z8Y4_MANLE CMP/dCMP-type deaminase domain
containing protein OS = Mandrillus leucophaeus OX = 9568 GN = APOBEC1
PE = 4 SV = 1
MTSEKGPSTGDPTLRRRIEPWEFDIFYDPRELRKEACLLYEIKWGMSPKIWRSS
GKNTTNHVEVNFIEKLTSERRFHSSISCSITWFLSWSPCWECSQAIREFLSQHP
GVTLVIYVARLFWHTDQQNRQGLRDLVNSGVTIQIMRASEYYHCWRNFVNYPP
GEEAHWPRYPPLWMMLYALELHCIILSLPPCLKISRRQQNHLTFFRLHLQNCHY
QTIPPHILLATGLIQPSVTWR
SEQ ID NO: 34
>tr|A0A087VMP5|A0A087VMP5_BALRE C->U-editing enzyme APOBEC-1
(Fragment) OS = Balearica regulorum gibbericeps OX = 100784 GN = N312_10691
PE = 4 SV = 1
RWKIQPDDFKRNYLPGKHPRVVYLLYEIRWSRGTIWRSWCSNNATQHAEINFL
ETCFLARTSVSCSITWVLSTTPCGKCSRRILEFLNAYPNVTLEIYAAKLFRHLDN
RNRQGLRNLAMKGVRIHIMNLADYSYFWKIFVAY
SEQ ID NO: 35
>tr|A0A087QNJ5|A0A087QNJ5_APTFO C->U-editing enzyme APOBEC-1
(Fragment) OS = Aptenodytes forsteri OX = 9233 GN = AS27_08049
PE = 4 SV = 1
RWKIRPNDFKRNYLPGQHPKVVYLLYEIRWSRGTIWRNWCSNNSTQHAEVNFL
ENCFKAMPSVSCSITWVLSTTPCGKCSRRILEFLRVHPNVTLEIYAAKLFKHLDI
RNRQGLRNLAMNGVIIRIMNLADYSYGWKRFVAY
SEQ ID NO: 36
>tr|A0A2Y9IYV0|A0A2Y9IYV0_ENHLU C->U-editing enzyme APOBEC-1
OS = Enhydra lutris kenyoni OX = 391180 GN = LOC111142361 PE = 4
SV = 1
MASDKGPSAGDATLRRRIEPWEFEVFFDPRELRKEACLLYEIQWGTSHKMWR
NTGKNTANHVELNFIEKFTSERRYCPSTHCSITWFLSWSPCWECCKAIRGFLSQ
HPSVTLVIYVTRLFWHMDPQNRQGLRDLLKSGVTVQIMRAPEYDHCWKNFVNY
PPGKEDHAPRYPELWMKLYELELYCIILSLPPCLKISRRNQNQLTLFRLTLQNC
HYQIIPPHILLDTGLIQLPVIWR
SEQ ID NO: 37
>tr|B2NIW5|B2NIW5_MUSPF Apolipoprotein B mRNA editing protein
OS = Mustela putorius furo OX = 9669 GN = APOBEC1 PE = 2 SV = 1
MASDKGPSAGDATLRRRIEPWEFEVFFDPRELRKEACLLYEIQWGTSHKMWR
NTGKNTANHVELNFIEKFTSERRYCPSTHCSITWFLSWSPCWECSKAIRGFLSQ
CPSVTLVIYVTRLFWHMDPQNRQGLRDLLKSGVTVRIMRAPEYDHCWKNFVNY
PPGKEDHWPRYPELWMKLYELELYCIILSLPPCLKISRRNQKQLTLFRLTLQNCH
YQIIPPHILLDTGLIQLPVIWR
SEQ ID NO: 38
>tr|A0A2Y9E587|A0A2Y9E587_TRIMA C->U-editing enzyme APOBEC-1
OS = Trichechus manatus latirostris OX = 127582 GN = LOC101361717
PE = 4 SV = 1
MTSEEADQRHSTMTSEKGPSTGDGTLRRRITPWEFEIFFDPRELRKETCLLYEI
KWGTSHRIWRNSGQNTTKHAEVNFIEKFTSERNFCPSVSCSITWFLSWSPCWE
CSKAIREFLSQHPNVILVIYVARLFHHMDQQNREGLRDLVLSGVTVQIMSVSEY
GHCWRNFVNYPPGEEARWPRYPPLWMMLYALELHCIILGLPPCLKISRRRQSQ
LTLFSLTPQNCHYQMIPPHILLATGLIQPYVTWR
SEQ ID NO: 39
>tr|G1LKL4|G1LKL4_AILME CMPidCMP-type deaminase domain-containing
protein OS = Ailuropoda melanoleuca OX = 9646 GN = APOBEC1 PE = 4 SV = 1
ISWSTGPSGGDATSRRRIEPWEFEVFFDPRQLRKEACLLYEIQWGTSRKIWRN
SGKNTTNHVEINFIEKFTLERQYCPSIHCSVTWFLSWSPCWECSKAIRAFLSQH
PSVTLVEYVARLFWHMEPQNROGLRDLINSGVTIQIMSVPEYDHCWRNFVNYPP
GKDHWPGYPVLWMKLYALELHCIILSLPPCLKISRRNQNQLTLFRLTLQNCHYQ
TIPPHVLLATGLIQLPVTWR
SEQ ID NO: 40
>tr|A0A093PWR2|A0A093PWR2_9PASS C->U-editing enzyme APOBEC-1
(Fragment) OS = Manacus vitellinus OX = 328815 GN = N305_14278
PE = 4 SV = 1
RWKIQPKDFKRNYLPGQHPQVVYLLYEIRWRNGSIWRNWFSNNRNQHAEVNF
LENCFSDVPPAPCSITWFLSTSPCGKCSRRILEFLRTHRNVTLEIYAAKLFRHQD
IRNRQGLCNLVMNGVTIHIMNLADYSYCWKRFVAY
SEQ ID NO: 41
>sp|Q9EQP0|ABEC1_MESAU C->U-editing enzyme APOBEC-1
OS = Mesocricetus auratus OX = 10036 GN = APOBEC1 PE = 2 SV = 1
MSSETGPVVVDPTLRRRIEPHEFDAFFDQGELRKETCLLYEIRWGGRHNIWRH
TGQNTSRHVEINFIEKFTSERYFYPSTRCSIVWFLSWSPCGECSKAITEFLSGHP
NVTLFIYAARLYHHTDQRNRQGLRDLISRGVTIRIMTEQEYCYCWRNFVNYPPS
NEVYWPRYPNLWMRLYALELYCIHLGLPPCLKIKRRHQYPLTFFRLNLQSCHYQ
RIPPHILWATGFI
SEQ ID NO: 42
>tr|A0A2K6PRF3|A0A2K6PRF3_RHIRO CMP/dCMP-type deaminase domain-
containing protein OS = Rhinopithecus roxellana OX = 61622 GN = APOBEC1
PE = 4 SV = 1
MSWKIWRSSGKNTTNHVEVNFIEKFTSERRFHSSISCSITWFLSWSPCWDCSQ
AIRKFLSQHPGVTLVIYVARLFWHTDQQNROGLRDLVNSGVTIQMMTASEYYH
CWRNFVNYPPGEEAHWPRYPPLWMMLYALELHCIILSLPPCLKISRRWQNHLT
FFRLRLQNCHYQTIPPHILLATGLIQPSVTANR
SEQ ID NO: 43
>tr|A0A0D9RBS4|A0A0D9RBS4_CHLSB Apolipoprotein B mRNA editing
enzyme catalytic subunit 1 OS = Chlorocebus sabaeus OX = 60711 GN = APOBEC1
PE = 4 SV = 1
MSRKIWRSSGKNTTNHVEVNFIEKLTSERRFHSSVSCSVTWFLSWSPCWECS
QAIREFLSQHPGVTLVIYVARLFWHTDQQNRQGLRDLVNSGVTIQIMRASEYYH
CWRNFVNYPPGEEAHWPRYPPLWMMLYALELHCIILSLPPCLKISRRWQNHLT
FFRLHLQNCHYQTIPPHILLATGLIQPPVTWR
SEQ ID NO: 44
>tr|A0A286XNR2|A0A286XNR2_CAVPO CMP/dCMP-type deaminase domain-
containing protein OS = Cavia porcellus OX = 10141 GN = APOBEC1
PE = 4 SV = 1
MASGTGPSTGDATLRRRIEPWQFEAYFDPRQLRKEACMLSEVRWGASPRTW
RESSLNTTSHVEINFIEKFTSGRSLRPAVRCSMTWFLSWSPCWECARAIREFLH
QHPNVSLVIYVARLYWHVDEQNROGLRDLVTSGVRVQIMSDSEYRHCWRNFV
NFPPGQEAGWPRFPPMWTTLYALELSCILLSLPPCLKISRRRQYRLIVFQLILQT
CHYRAIPPQVLSAAELMHPLVAWC
SEQ ID NO: 45
>tr|A0A2Y9HAT6|A0A2Y9HAT6_NEOSC C->U-editing enzyme APOBEC-1
OS = Neomonachus schauinslandi OX = 29088 GN = APOBEC1 PE = 4 SV = 1
MASDKGPSAGDATLRRRIKPWEFEVFFDPRELRKETCLLYEIQWGTSHKIWRN
SGKNTANHVEINFIEKFTSERQYCPSIRCSITWFLSWSPCWECSKAIRGFLSQH
PSVTLVEYVARLFWHIVIDPQNRQGLRDLINSGVTIQIWIRVPEYDHCWRNFVNYLP
GKEDHWPRYPVLWMKLYALELHCIILSLPPCLRISRRQNQLTLFTLTLQNCHYQ
MIPPHILLATGLIQVPVTWK
SEQ ID NO: 46
>tr|A0A091XJL0|A0A091XJL0_OPIHO C->U-editing enzyme APOBEC-1
(Fragment) OS = Opisthocomus hoazin OX = 30419 GN = N306_09750
PE = 4 SV = 1
RWKVQPNDFKRNYLPGQHPKVVYILYEIRWSRGTIWRNWCTNNSTQHAEVNF
LENCFKAMPSVSCSITWVLSTTPCGKCSKRIQDFLRIYPNVTLEIHAAKLFKHLD
TRNREGLRNLAKDGVIIHIMNLADYSYWWKRFVAY
SEQ ID NO: 47
>tr|F6WR88|F6WR88_HORSE Apolipoprotein B mRNA editing enzyme catalytic
subunit 1 OS = Equus caballus OX = 9796 GN = APOBEC1 PE = 4 SV = 2
MSHNIWRYSGKNTTKHVEINFIEKFTSERHLRPSISCSIVWFLSWSPCWECSKAI
REFLS
QHPNVTLVIYVARLFQHMDRLNRQGLRDLINSGVTIQIMRTSEYDHCWRNFVNY
PPGKEAHWPRYPLLWMKLYALELHCIILSLPPCLMISRRCQNQLTFFRLTLQNC
HYQMIPPHILLATGLVQLPVTWR
SEQ ID NO: 48
>sp|P41238|ABEC1_HUMAN C->U-editing enzyme APOBEC-1 OS = Homo
sapiens OX = 9606 GN = APOBEC1 PE = 1 SV = 3
MTSEKGPSTGDPTLRRRIEPWEFDVFYDPRELRKEACLLYEIKWGMSRKIWRS
SGKNTTNHVEVNFIKKFTSERDFHPSMSCSITWFLSWSPCWECSQAIREFLSR
HPGVTLVIYVARLFWHMDQQNRQGLRDLVNSGVTIQIMRASEYYHCWRNFVN
YPPGDEAHWPQYPPLWMMLYALELHCIILSLPPCLKISRRWQNHLTFFRLHLQN
CHYQTIPPHILLATGLIHPSVAWR
SEQ ID NO: 49
>tr|A0A091RU17|A0A091RU17_NESNO C->U-editing enzyme APOBEC-1
(Fragment) OS = Nestor notabilis OX = 176057 GN = N333_10787 PE = 4
SV = 1
RWKIQPNDFKRNYLPYQHPKVVCLLYEIRWNRGTIWRSWCSNNSTQHAEVNFL
ENCFKAKPSVSCSITWVLSTTPCGECSRRILDFLSVYPNVTLKIYAAKLFKHLDN
RNRQGLWNLANNRVIIRIMNLEDYNYYWKRFVAY
SEQ ID NO: 50
>tr|A0A091IWL9|A0A091IWL9_EGRGA C->U-editing enzyme APOBEC-1
(Fragment) OS = Egretta garzetta OX = 188379 GN = Z169_08812 PE = 4
SV = 1
RWKIQPNDFKRNYLPGQHPKVVYLLYEIRWSRGTIWRNWCSNNSTQHAEVNF
LENCFKAMPSVSCSITWVLSTTPCGKCSRRILEFLRVHPSVTLEIYAAKLFKHLDI
RNRQGLRNLAMNGVIIHIMNLADYSYWWKIFVAY
SEQ ID NO: 51
>tr|A0A2K5DG70|A0A2K5DG70_AOTNA CMP/dCMP-type deaminase domain-
containing protein OS = Aotus nancymaae OX = 37293 GN = APOBEC1 PE = 4
SV = 1
MTPEEEVQRQSTMTSERGPSTGDPTLRRRIEPWEFCISYDPKELCKETCLLYEI
KWGTSWKIWRSSGKNTTNHVEVNFIEKFMSERHFHSSISCSITWFLSWSPCWE
CSQAIREFLSRHPGVTLVIYVARLFQHMDRQNRQGLRDLVNSGVTIQIMRASEY
YHCWRNFVNYPPGEEAHWPRYPPLWMMLYALELHCIILGLPPCLKISRRWQNR
LTFFRLHLQNCHYQMIPQHILFATGLIQPPVTWR
SEQ ID NO: 52
>sp|P51908|ABEC1_MOUSE C->U-editing enzyme APOBEC-1 OS = Mus
musculus OX = 10090 GN = Apobec1 PE = 1 SV = 1
MSSETGPVAVDPTLRRRIEPHEFEVFFDPRELRKETCLLYEINWGGRHSVWRH
TSQNTSNHVEVNFLEKFTTERYFRPNTRCSITINFLSWSPCGECSRAITEFLSRH
PYVTLFIYIARLYHHTDQRNRQGLRDLISSGVTIQIMTEQEYCYCWRNFVNYPPS
NEAYWPRYPHLWVKLYVLELYCIILGLPPCLKILRRKQPQLTFFTITLQTCHYQRI
PPHLLWATGLK
SEQ ID NO: 53
>tr|G5BPM8|G5BPM8_HETGA C->U-editing enzyme APOBEC-1 (Fragment)
OS = Heterocephalus glaber OX = 10181 GN = GW7_17308 PE = 4 SV = 1
RRRIEPWQFEASFDPRQLRRETCLLSEVRWGTSPRAWRGCSLNTARHAEVSF
MDRLTSEGRLRGPVRCSITWFLSWSPCGACAQAIGEFLRQHPNVSLVIYIARLF
WHVDEQNRQGLRDLVTRGVRMQVMSDPEFAHCWRNFVNYSPGQEARWPQV
PPVWTWLYSLELHCILLNLPPCLKISRRHHNQLTFFQLILQNCHYQAIPSPVLLAS
GLIHPFVTW
SEQ ID NO: 54
>tr|A0A091QEK6|A0A091QEK6_MERNU C->U-edit ng enzyme APOBEC-1
(Fragment) OS = Merops nubicus OX = 57421 GN = N331_01832 PE = 4
SV = 1
RWKIEPDEFKTNYSPDHRPRVVYLLYEIRWRRGTIWRNWCSNNIDQHAEVNFL
ENCFKAK
PSVSCSITWFLSTAPCAKCSRRILKFLTAHPKVTLEIYAAKLFRHLEIRNRQGLMD
LAVN
GVILRIMNLADYSYCWKQFVAY
SEQ ID NO: 55
>tr|A0A093LP85|A0A093LP85_FULGA C->U-editing enzyme APOBEC-1
(Fragment) OS = Fulmarus glacialis OX = 30455 GN = N327_13724 PE = 4
SV = 1
RWKIQPNDFKRNFLPSKYPKVVYLLYEIRWSSGTIWRSWCSNNSTQHAEVNFL
ENCFKAM
PSVSCSITWVLPITPCGKCSKKILEFLSVHPNVTLEIYAAKLFRHLDIRNQQGLRN
LAMN
GVIIRIMNLADYSYSWKRFVAY
SEQ ID NO: 56
>tr|G1QZV0|G1QZV0_NOMLE CMP/dCMP-type deaminase domain-containing
protein OS = Nomascus leucogenys OX = 61853 GN = APOBEC1 PE = 4 SV = 1
MTSEKGPSTGDPTLRRRIEPWEFDVFYDPRELRKEACLLYEIKWGMSQKIWRS
SGKNTTN
HVEVNFIKKFTSEGRFQSSISCSITWFLSWSPCWECSQAIREFLSQHPGVTLVIY
VARLF
WHMDQQNRQGLRDLVNSGVTIQIMRASEYYHCWRNFVNYPPGDEAHWPRYP
PLWMMLYAL
ELHCIILSLPPCLKISRRWQNHLTFFRLHLQNCHYQTIPPHILLATGLIHPSVTWR
SEQ ID NO: 57
>tr|A0A096MWB4|A0A096MWB4_PAPAN CMP/dCMP-type deaminase domain-
containing protein OS = Papio anubis OX = 9555 GN = APOBEC1 PE = 4 SV = 2
MTSEKGPSTGDPTLRRRIEPWEFDIFYDPRELRKEACLLYEIKWGMSPKIWRSS
GKNTTN
HVEVNFIEKLTSERRFHSSISCSITWFLSWSPCWECSQAIREFLSQHPGVTLVIY
VARLF
WHTDQQNRQGLRDLVNSGVTIQIMTASEYYHCWRNFVNYPPGEEAHWPRYP
PLWMMLYAL
ELHCIILSLPPCLKISRRWQNHLTFFRLHLONCHYQTIPPHILLATGLIQPSVTWR
SEQ ID NO: 58
>sp|Q9TUI7|ABEC1_MONDO C->U-editing enzyme APOBEC-1
OS = Monodelphis domestica OX = 13616 GN = APOBEC1 PE = 1 SV = 1
MNSKTGPSVGDATLRRRIKPWEFVAFFNPQELRKETCLLYEIKWGNQNIWRHS
NQNTSQH
AEINFMEKFTAERHFNSSVRCSITWFLSWSPCWECSKAIRKFLDHYPNVTLAIFI
SRLYW
HMDQQHRQGLKELVHSGVTIQIMSYSEYHYCWRNFVDYPQGEEDYWPKYPYL
WIMLYVLE
LHCIILGLPPCLKISGSHSNQLALFSLDLQDCHYQKIPYNVLVATGLVQPFVTWR
SEQ ID NO: 59
>tr|A0A1S3FTE2|A0A1S3FTE2_DIPOR C->U-editing enzyme APOBEC-1
OS = Dipodomys ordii OX = 10026-GN = Apobec1 PE = 4 SV = 1
MHHSARLPPNCIVSRYANAPWTVLPLPLPPTEAPATGDDTLRRRIEPWEFEAFF
NPQELR
REACLLYQITWSSHKVWRETAKNTVDSHVEVNFIQNLTAGRYCRPSTRCSILWF
LSWSPC
SSCSKAIRLFLSQHPGVSLVIYVARLFQHMDPQNRQGLRELIHSGVTIQVMRPQ
EYDYCWKNFVNYPPGQEEHWPRYPVQCMTLYNLELYCIIHNLPPCVRISKQRQ
SQLAFFSLGLENVHYQRIPPPLLLLTGLVFVFPWK
SEQ ID NO: 60
>tr|A0A2U3WPA5|A0A2U3WPA5_ODORO C->U-editing enzyme APOBEC-1
OS = Odobenus rosmarus divergens OX = 9708 GN = APOBEC1 PE = 4 SV = 1
MASDKGPSAGDATLRRRIEPWEFEVFFDPRELRKEACLLYEIQWGTSHKIWRN
SGKNTSNHVEINFIEKFTSERQYCPSIHCSITWFLSWSPCWECSEAIRGFLSQH
PSVTLVEYVARLFWHIVIDPQNRQGLRDLINSGVTIQIMRVPEYDHCWRNFVNYP
PGKEDHWPRYPVLWMKLYALELHCIILSLPPCLRISRRQNQLTLFRLTLQNCHY
QMIPPHILLATGLIQVPVTWK
SEQ ID NO: 61
>tr|A0A1V4JAP2|A0A1V4JAP2_PATFA C->U-editing enzyme APOBEC-1
OS = Patagioenas fasciata monilis OX = 372326 GN = APOBEC1 PE = 4 SV = 1
MRRKKPSGMYISKRALKDNFDPHKFPHDTYLLCKLQWGDTGRSWIHWIRKDR
YHAEVYFLEKIFKMRRSKNYVNCSITWYLSWSPCVRCCCEILNFLEKHSYVNIDI
YVARLYKIQNSEVREGLKKLVSSKKVTIAVMEIKDYTYCWKNFIQGDADDDSWT
VDFQSAITKNRLKLKDVFEFLKSHPNVTLEIYAAKLFKHLDIRNREGLRNLAKNG
VIIHIMNLADYSYWWKIFVTRQHGEDDYLPWSFALHIFLNCIEFQQILLVSRHLKE
SLRVKSNEKAQEKEVWRIPAMVLAEMIVGKMNRDLMLHEQRANRARNCKGLW
CYIVPL
SEQ ID NO: 62
>tr|A0A2K5JKV4|A0A2K5JKV4_COLAP CMP/dCMP-type deaminase domain-
containing protein OS = Colobus angolensis palliatus OX = 336983 GN = APOBEC1
PE = 4 SV = 1
PSTGDPTLRRRIEPWEFDIFYDPRELRKEACLLYEIKWGMSQKIWRSSGKNTTN
HVEVNFIEKLTSERRFHSSVSCSITWFLSWSPCWECSQAIREFLSQHPGVTLVI
YVARLPAIHTDQQNROGLRDLVNSGVTIQMMTASEYYHCWRNFVNYPPGEEA
HWPRYPPLWMMLYALELHCIILSLPPCLKISRRWQNHLTFFRLHLQNCHYQTIP
PHILLATGLIQPSVTWR
SEQ ID NO: 63
>tr|A0A1U7U8J6|A0A1U7U8J6_TARSY LOW QUALITY PROTEIN: C->U-editing
enzyme APOBEC-1 OS = Tarsius syrichta OX = 1868482 GN = APOBEC1 PE = 4
SV = 2
MLTALMEEVQDTMRFGRRAFFLSNSVGIWVLFDISISXSTGPSMGDPTLRRRIE
PWEFEVLFDPRELRKEACLLYEIKWGTSCKIWRNSGKNTSNHAEVNFLEKFTSE
RHFCSSTSYSITWFLSWSPCWECSRAIREFLSQHPRVTLVIYVARLFWHMEPQ
NRQGLRDLINSGVTIQIMRDSGKSNKQIIRIVCERTW
SEQ ID NO: 64
>tr|F1SLW4|F1SLW4_PIG CMP/dCMP-type deaminase domain-containing
protein OS = Sus scrofa OX = 9823 GN = APOBEC1 PE = 4 SV = 2
MASDRGPSAGDATSRRRIEPWEFEVFFDPRELRKETCLLYELQWGRSRDTWR
HTGKNTTNHVERNFLAKITSERHFHPSVHCSIVWFLSWSPCWECSEAIREFLDQ
HPSVTLVIYVARLFQHMDPQNRQGLRDLVNHGVTIQIMGAPEYDYCWRNFVNY
PPGKEAHWPRFPPVWMTLYALELHCIILGLPPCLKISRRCQNQLTFFRLTLQNC
HYQTIPPHILLATGLIQLPVIYR
SEQ ID NO: 65
>tr|A0A2K6BGI5|A0A2K6BGI5_MACNE CMP/dCMP-type deaminase domain-
containing protein OS = Macaca nemestrina OX = 9545 GN = APOBEC1 PE = 4
SV = 1
MTSEKGPSTGDPTLRRRIEPWEFDIFYDPRELRKEACLLYEIKWGMSPKIWRSS
GKNTTNHVEVNFIEKLTSERRFHSSISCSITWFLSWSPCWECSQAIREFLSQHP
GVTLVIYVARLFWHTDQQNRQGLRDLVNSGVTIQIMRASEYYHCWRNFVNYLP
GEEAHWPRYPPLWMMLYALELHCIILSLPPCLKISRRWQNHLTFFRLHLQNCHY
QMIPPHILLATGLIQPSVTWR
SEQ ID NO: 66
>sp|P47855|ABEC1_RABIT C->U-editing enzyme APOBEC-1 OS = Oryctolagus
cuniculus OX = 9986 GN = APOBEC1 PE = 1 SV = 1
MASEKGPSNKDYTLRRRIEPWEFEVFFDPQELRKEACLLYEIKWGASSKTWRS
SGKNTTNHVEVNFLEKLTSEGRLGPSTCCSITWFLSWSPCWECSMAIREFLSQ
HPGVTLIIFVARLFQHMDRRNRQGLKDLVTSGVTVRVMSVSEYCYCWENFVNY
PPGKAAQWPRYPPRWMLMYALELYCIILGLPPCLKISRRHQKQLTFFSLTPQYC
HYKMIPPYILLATGLLQPSVPWR
SEQ ID NO: 67
>sp|P38483|ABEC1_RAT C->U-editing enzyme APOBEC-1 OS = Rattus
norvegicus OX = 10116 GN = Apobec1 PE = 1 SV = 1
MSSETGPVAVDPTLRRRIEPHEFEVFFDPRELRKETCLLYEINWGGRHSIWRHT
SONTNKHVEVNFIEKFTTERYFCPNTRCSITWFLSWSPCGECSRAITEFLSRYP
HVTLFIYIARLYHHADPRNRQGLRDLISSGVTIQMATEQESGYCWRNFVNYSPSN
EAHWPRYPHLWVRLYVLELYCIILGLPPCLNILRRKQPQLTFFTIALQSCHYQRL
PPHILWATGLK
SEQ ID NO: 68
>tr|A0A091M4D7|A0A091M4D7_CARIC C->U-editing enzyme APOBEC-1
(Fragment) OS = Cariama cristata OX = 54380 GN = N322_12137 PE = 4
SV = 1
RWKIQPDDFKRNYLPGQHPEVVYLLYEIKWNSGTIWRNWCSNNPTQHAEVNFL
ENHFNVMSSVSCSITWGISTTPCGKCSRRILEFLTTHPNVTLEIYAAKLFKHLDIR
NROGLRNLAMNGVVICIMNLADYSYRNKTFVAY
SEQ ID NO: 69
>tr|A0A093F3R4|A0A093F3R4_GAVST C->U-editing enzyme APOBEC-1
(Fragment) OS = Gavia stellata OX = 37040 GN = N328_12441 PE = 4
SV = 1
RWKIQPNDFKRNYLPAQHPKVVYLLYEIRWSRGTIWRNWCSNNSTQHAEVNFL
ENCFKAMPSVSCSITWFLSTTPCGKCSRRILTFLREHPNVTLEIYAAKLFKHLDV
RNQQGLRNLDRNGVIIRIMNFADYSYCWKRFVAY
SEQ ID NO: 70
>tr|G7N5W0|G7N5W0_MACMU C->U-editing enzyme APOBEC-1 (Fragment)
OS = Macaca mulatta OX = 9544 GN = EGK_03318 PE = 4 SV = 1
GPSTGDPTLRRRIEPWEFDIFYDPRELRKEACLLYEIKWGMSPKIWRSSGKNTT
NHVEVNFIEKLTSERRFHSSISCSITWFLSWSPCWECSQAIREFLSQHPGVTLVI
YVARLFWHTDQQNRQGLRDLVNSGVTIQIMRASEYYHCWRNFVNYPPGEEAH
WPRYPPLWMMLYALELHCIELSLPPCLKISRRWQNHLTFFRLHLQNCHYQMIPP
HILLATGLIQPSVTWR
SEQ ID NO: 71
>tr|A0A091MEP8|A0A091MEP8_9PASS C->Uediting enzyme APOBEC-1
(Fragment) OS = Acanthisitta chloris OX = 57068 GN = N310_12928
PE = 4 SV = 1
RWKIQPNDFQRNYLPDQHPQAVYLLYEFRWRRGSIWRKWCSNNRAQHAEVN
FLENCFNGIPPVPCSITWFLSTTPCGNCSRRILEFLRLHPNVTLEIYAAKLFRHTD
IRNRKGLYNLAMNGVIIRIMNLADYSYCWRNFVAY
SEQ ID NO: 72
>tr|A0A2I0LXZ8|A0A2I0LXZ8_COLLI Apolipoprotein B mRNA editing enzyme,
catalytic polypeptide 1 OS = Columba livia OX = 8932 GN = APOBEC1 PE = 4
SV = 1
MAAVTNRDSACRENNQRWKIQPNDFRRNYLPDKQPRVVYLLYEIRWRRGTIW
RNWCSNNPNQHAEVNFLKNYFNAMPSVSCSITWVLSTTPCGKCSIKIMEFLKLH
PNVTLEIYAAKLFKHLDIRNREGLRNLAKNGVIIHIMNLADYSYWWKIFVTRQHG
EEDYLPWSFALHIFLNCIEFQQILLGLPPLLPNFKY
SEQ ID NO: 73
>tr|W5NVH9|W5NVH9_SHEEP CMP/dCMP-type deaminase domain-containing
protein OS = Ovis aries OX = 9940 GN = APOBEC1 PE = 4 SV = 1
MASDRGPPAGDPURRRIEPLEFEFSFDPRNFCKEAYLLYEIQWGNSRDVWRH
SGKNTTKHVERNFIEKIASERHFRPSISCSISWYLSWSPCWECSKAIREFLNQH
PNVTLVIYIARLFQHMDPQNRQGLKDLFHSGVTIQVMRDPEYDYCWRNFVNYP
QGKEAHWPRYPPLWMNLYALELYCIISGLPPCLQISRRHQNQLRVFRLIPQNCH
YQMIPPCILLATGMIQLPVTWRWIE
SEQ ID NO: 74
>tr|H0XVG8|H0XVG8_OTOGA CMP/dCMP-type deaminase domain-containing
protein OS = Otolemur garnettii OX = 30611 GN = APOBEC1 PE = 4 SV = 1
ISWSTGISTGDPTLRRRIEPWEFEVFFDPRELRKETCLLYEIKWGTSHKIWRSTA
RNTTSHAEMNFIEKFTSERCSDAPISCSITWFLSWSPCWECSKNREFVSRHPS
VTLVIYVARLYWHMDQQNRQGLRDLISSGVTVQIMRVSEYCHCWRNFVNYLPG
KEAHCPRCPPLWMTLYALELHCIILSLPPCLKISRGHQNQLTLFRLTLQNCHYQT
IPPHVLLATGLIQPYVTWR
SEQ ID NO: 75
>tr|A0A2B4RXQ3|A0A2B4RXQ3_STYPI C->U-editing enzyme APOBEC-1
OS = Stylophora pistillata OX = 50429 GN = APOBEC1 PE = 4 SV = 1
MASVTELRTPDDFLAELLWTGVTGRTWPNRTFLIVSIKAKDGKPIFGKRFKNRY
PEHAEIIMLRNSNFSDVVEKNHDIDITLTLNYSPCSSCACILKEFYVNNSNIKCFTI
QFSFIYYKEDMKNKTGLQNLEEAGVTLQAMNAESWREVGIDLESFTPEDKEKIN
KRDKDTANDLNEVLSSKQDQDASVDELSSQLNAKLRAKET
SEQ ID NO: 76
>tr|A0A2K5L2J6|A0A2K5L2J6_CERAT CMP/dCMP-type deaminase domain-
containing protein OS = Cercocebus atys OX = 9531 GN = APOBEC1 PE = 4
SV = 1
MTPEEEVQRQSTMTSEKGPSTGDPTLRRRIEPWEFDIFYDPRELRKEACLLYEI
KWGMSPKIWRSSGKNTTNHVEVNFIEKLTSERRFHSSISCSITWFLSWSPCWE
CSQAIREFLSQHPGVTLVIYVARLFWHTDQQNRQGLRDLVNSGVTIQIMRASEY
YHCWRNFVNYPPGEEAHWPRYPPLWMIVILYALELHCIILSLPPCLKISRRQQNH
LTFFRLHLQNCHYQTIPPHILLATGLIQPSVTWR
SEQ ID NO: 77
>tr|A0A2Y9T649|A0A2Y9T649_PHYMC C->U-editing enzyme APOBEC-1
isoform X1 OS = Physeter macrocephalus OX = 9755 GN = APOBEC1 PE = 4
SV = 1
MIICWSTGPSAGDATSRRRIEPWEFEVSFDPREFCKEARLLYEIKWGKSQDVW
RHSGKNTTKHVECNFIEKMTSERHFHPSISCCIIWFLSWSPCWECSKAIREFLN
QHPSVTLVIYIARLFQHTDPQNRQGLRDLIHSGVTLQIMGPPEYDYCWRNFVNY
PPGKEAHWPRYPPLWMKLYALELHCIILGLPPCLKISRRCQNQLTWFRLILQNC
HYQMIPPHILLGTGLIQLPVAWR
SEQ ID NO: 78
>tr|H2NGD0|H2NGD0_PONAB Apolipoprotein B mRNA editing enzyme catalytic
subunit 1 OS = Pongo abelii OX = 9601 GN = APOBEC1 PE = 4 SV = 1
MTPEEEVORQSTMTSEKGPSTGDPTLRRRIESWEFDVFYDPRELRKETCLLYEI
KWGMSRKIWRSSGKNTTNHVEVNFIKKFTSERRFHSSISCSITWFLSWSPCWE
CSQAIREFLSQHPGVTLVIYVARLFWHMDQRNRQGLRDLVNSGVTIQIMRASEY
YHCWRNFVNYPPGDEAHWPQYPPLWMMLYALELHCIILSLPPCLKISRRWQNH
LAFFRLHLQNCHYQTIPPHILLATGLIHPSVTWK
SEQ ID NO: 79
>tr|A0A093JI54|A0A093JI54_EURHL C->U-editing enzyme APOBEC-1
(Fragment) OS = Eurypyga helias OX = 54383 GN = N326_10046 PE = 4 SV = 1
RWKIQPNDFKRNYMPSQYPKVVYLLYEIRWSRGTVWRNWCSNSFTQHAEVNF
LENYFKPMPSVSCSITVVVLSTTPCGKCSRRILEFLRVHPNVTLEIYAAKLFKHLDI
RNRQGLRDLAMNGVTIRIMNLADYSFCWKRFVAY
SEQ ID NO: 80
>tr|G3W4I1|G3W4I1_SARHA Apolipoprotein B mRNA editing enzyme catalytic
subunit 1 OS = Sarcophilus harrisii OX = 9305 GN = APOBEC1 PE = 4 SV = 1
MGDATLRRRIKSWEFEAFFNPQELRKETCLLYEIKWGASHNIWRSSNQNTTQH
AEINFMEKFTSERNFKPSVKCSITWFLSWSPCWRCSKAIREFLNQYPNVTLVIFV
SRLYWHMEQQHRQELKELVCSGVTIQIMNYSEYRHCWRNFVDYLPEEEDHWP
KYPTLWIMLYVLELHCIILGLPPCLKISVRHSDQLVLFSLDLQDCHYQKIPYHVLV
ATGIIRPFVTWR
SEQ ID NO: 81
>tr|A0A2U3Y3M5|A0A2U3Y3M5_LEPWE c->U-editing enzyme APOBEC-1
OS = Leptonychotes weddellii OX = 9713 GN = APOBEC1 PE = 4 SV = 1
MASDKGPSAGDATLRRRIKPWEFEVFFDPRELRKETCLLYEIQWGTSHKIWRN
SGKNTANHVEINFIEKFTSERQYCPSIRCSITWFLSWSPCWECSKAIRGFLSQH
PSVTLVEYVARLFWHIVIDPQNRQGLRDLINSGVTIQIMRVPEYDHCWRNFVNYLP
GKEDHWPRYPVLWMKLYALELHCIILPIEMPGKIRDAPNNMEIFSLFVGRYIPKT
KFHVTCLLSDVRNDDSHLDKTAPKWIRFDSLQPVASDPSAEHWKMKLPGRDD
KTAVVVGTVTEDVACAQGAKLYLCALRVHGHAQRHFLKGRDEILALDQLALDSP
QGLWRQPDLRSHPLKG
SEQ ID NO: 82
>tr|A0A1S3AN78|A0A1S3AN78_ERIEU C->U-editing enzyme APOBEC-1-like
OS = Erinaceus europaeus OX = 9365 GN = LOC103126721 PE = 4 SV = 1
RRIEPWEFEDFFDPRQFRPETCLLYEVRWGSSRNAWRSTARNTTRHAEVNFL
ERFAAERHFDKPVSCSITINFLSWSPCWECSQAIGAFLSQHPQVTLAIHVTRLFH
HEDEQNRQGLRDLLARGVTLQVMGDSEYAHCVVRTFVNSPPGAEGHYPRYPS
DFTRLYALELHCIILGLPPCLEILRRYQNQFTLFRLVPQNCHYQMIPHLNFFVVRH
YFF
SEQ ID NO: 83
>tr|A0A091PSV3|A0A091PSV3_HALAL C->U-editing enzyme APOBEC-1
(Fragment) OS = Haliaeetus albicilla OX = 8969 GN = N329_07103 PE = 4 SV = 1
RWKLQPNDFKRNYLPGQHPKVVYLLYEIRWSRGTIWRNWCSNNSTQHAEVNF
LENCFKATPSVSCSITVVVLSTTPCGKCSRRILEFLRVHPNVTLEIYAAKLFKHLDI
RNRKGLRDLAMNGVIIRIMNLSDYSYCWKTFVAY
SEQ ID NO: 84
>tr|F7F6M6|F7F6M6_CALJA Apolipoprotein B mRNA editing enzyme catalytic
subunit 1 OS = Callithrix jacchus OX = 9483 GN = APOBEC1 PE = 4 SV = 2
RRIEPWEFYISYDPKELCKETCLLYEIKWGMSWKIWRSSGKNTTNHVEINFIEKF
TSERH
FHLSVSCSITWFLSWSPCWECSQAIREFLSQHPGVTLVIYVARLFQHMDQQNR
QGLRDLVNSGVTIQMMTVSEYYHCWRNFVNYPPGEEAHWPRHPPLWLMLYAL
ELHCIILGLPPCLKISRRRQNRLTFFRLHLQNCHYQMIPRHILLATGLIQPSVTWR
SEQ ID NO: 85
>tr|L8IDZ0|L8IDZ0_9CETA C->U-editing enzyme APOBEC-1 OS = Bos mutus
OX = 72004 GN = M91_02456 PE = 4 SV = 1
MIISWSTGPPAGDPTLRRRIEPWEFEFSFDPRKFCKEACLLYEIQWGNNRDVW
RHSGKNTTKHVERNFIEKIASERYFCPSIRCFIFWYLSWSPCVVECSKAIREFLNQ
HPNVTLVIYIARLFQHMDPQNRQGLKDLVQSGVTIQVMRAPEYEYCWRNFVNY
PRGKEAHWPRYPPLWIMNLYALELYCIILGLPPCLHISRRYQNQLIVFRLTLQNCH
YQMIPPYILLATGMVQIPMTVVR
SEQ ID NO: 86
>tr|A0A093CIQ8|A0A093CIQ8_9AVES C->U-editing enzyme APOBEC-1
(Fragment) OS = Pterocles gutturalis OX = 240206 GN = N339 03265 PE = 4
SV = 1
RWKIQPNYFKINNLPGQHPRVVOLLYAIRWSRSTLWKSWCSNNSTQHAEVNFL
ENCFKGNPSVFCFMTWPFFHTTPHGKCCRRTPEFLGVHPNVTLKIRAAKLFKH
LDRYNQQGLRNVAMNGVVIRIINL
SEQ ID NO: 87
>sp|Q9GZX7|AICDA HUMAN Single-stranded DNA cytosine deaminase
OS = Homo sapiens CTX = 9606 GN = AICDA PE = 1 SV = 1
MDSLLMNRRKFLYQFKNVRWAKGRRETYLCYVVKRRDSATSFSLDFGYLRNK
NGCHVELLFLRYISDWDLDPGRCYRVTWFTSWSPCYDCARHVADFLRGNPNL
SLRIFTARLYFCEDRKAEPEGLRRLHRAGVQIAIMTFKDYFYCWNTFVENHERT
FKAWEGLHENSVRLSRQLRRILLPLYEVDDLRDAFRTLGL
SEQ ID NO: 88
>sp|Q9Y235|ABEC2_HUMAN C->U-editing enzyme APOBEC-2 OS = Homo
sapiens OX = 9606 GN = APOBEC2 PE = 1 SV = 1
MAQKEEAAVATEAASQNGEDLENLDDPEKLKELIELPPFEIVTGERLPANFFKF
QFRNVEYSSGRNKTFLCYVVEAQGKGGQVQASRGYLEDEHAAAHAEEAFFNTI
LPAFDPALRYNVTWYVSSSPCAACADRIIKTLSKTKNLRLLILVGRLFMWEEPEI
QAALKKLKEAGCKLRIMKPQDFEYVWQNFVEQEEGESKAFQPWEDIQENFLYY
EEKLADILK
SEQ ID NO: 89
>sp|P31941|ABC3A_HUMAN DNA dC->dU-editing enzyme APOBEC-3A
OS = Homo sapiens OX = 9606 GN = APOBEC3A PE = 1 SV = 3
MEASPASGPRHLMDPHIFTSNFNNGIGRHKTYLCYEVERLDNGTSVKMDQHR
GFLHNQAKNLLCGFYGRHAELRFLDLVPSLQLDPAQIYRVTWFISWSPCFSWG
CAGEVRAFLQENTHVRLRIFAARIYDYDPLYKEALQMLRDAGAQVSIMTYDEFK
HCWDTFVDHQGCPFQPWDGLDEHSQALSGRLRAILQNQGN
SEQ ID NO: 90
>sp|Q9UH17|ABC3B_HUMAN DNA dC->dU-editing enzyme APOBEC-3B
OS = Homo sapiens OX = 9606 GN = APOBEC3B PE = 1 SV = 1
MNPQIRNPMERMYRDTFYDNFENEPILYGRSYTWLCYEVKIKRGRSNLLWDTG
VFRGQVYFKPQYHAEMCFLSWFCGNQLPAYKCFQITWFVSWTPCPDCVAKLA
EFLSEHPNVTLTISAARLYYYWERDYRRALCRLSQAGARVTIMDYEEFAYCWE
NFVYNEGQQFMPWYKFDENYAFLHRTLKEILRYLMDPDTFTFNFNNDPLVLRR
RQTYLCYEVERLDNGTWVLMDQHMGFLCNEAKNLLCGFYGRHAELRFLDLVP
SLQLDPAQIYRVTWFISWSPCFSWGCAGEVRAFLQENTHVRLRIFAARIYDYDP
LYKEALQMLRDAGAQVSIMTYDEFEYCWDTFVYRQGCPFQPWDGLEEHSQAL
SGRLRAILQNQGN
SEQ ID NO: 91
>sp|Q9NRW3|ABC3C_HUMAN DNA dC->dU-editing enzyme APOBEC-3C
OS = Homo sapiens OX = 9606 GN = APOBEC3C PE = 1 SV = 2
MNPQIRNPMKAMYPGTFYFQFKNLWEANDRNETWLCFTVEGIKRRSVVSWKT
GVFRNQVDSETHCHAERCFLSWFCDDILSPNTKYQVTWYTSWSPCPDCAGEV
AEFLARHSNVNLTIFTARLYYFQYPCYQEGLRSLSQEGVAVEINADYEDFKYCWE
NFVYNDNEPFKPWKGLKTNFRLLKRRLRESLQ
SEQ ID NO: 92
>sp|Q96AK3|ABC3D_HUMAN DNA dC->dU-editing enzyme APOBEC-3D
OS = Homo sapiens OX = 9606 GN = APOBEC3D PE = 1 SV = 1
MNPQIRNPMERMYRDTFYDNFENEPILYGRSYTWLCYEVKIKRGRSNLLWDTG
VFRGPVLPKRQSNHRQEVYFRFENHAENACFLSWFCGNRLPANRRFQITWFVS
WNPCLPCVVKVTKFLAEHPNVTLTISAARLYYYRDRDWRWVLLRLHKAGARVKI
MDYEDFAYCWENFVCNEGQPFMPWYKFDDNYASLHRTLKEILRNPMEANAYPH
IFYFHFKNLLKACGRNESWLCFTIVIEVTKHHSAVFRKRGVFRNQVDPETHCHAE
RCFLSWFCDDILSPNTNYEVTWYTSWSPCPECAGEVAEFLARHSNVNLTIFTA
RLCYFWDTDYQEGLCSLSQEGASVKIMGYKDFVSCWKNFVYSDDEPFKPWKG
LQTNFRLLKRRLREILQ
SEQ ID NO: 93
>sp|Q8IUX4|ABC3F_HUMAN DNA dC->dU-editing enzyme APOBEC-3F
OS = Homo sapiens OX = 9606 GN = APOBEC3F PE = 1 SV = 3
MKPHFRNTVERMYRDTFSYNFYNRPILSRRNTVWLCYEVKTKGPSRPRLDAKI
FRGQVYSQPEHHAEMCFLSWFCGNQLPAYKCFQITWFVSWTPCPDCVAKLAE
FLAEHPNVTLTISAARLYYYWERDYRRALCRLSQAGARVKIMDDEEFAYCWEN
FVYSEGQPFMPWYKFDDNYAFLHRTLKEILRNPMEAMYPHIFYFHFKNLRKAY
GRNESWLCFTMEVVKHHSPVSWKRGVFRNQVDPETHCHAERCFLSWFCDDIL
SPNTNYEVTWYTSWSPCPECAGEVAEFLARHSNVNLTIFTARLYYRAIDTDYQE
GLRSLSQEGASVEIMGYKDFKYCWENFVYNDDEPFKPWKGLKYNFLFLDSKLQ
EILE
SEQ ID NO: 94
>sp|Q9HC16|ABC3G_HUMAN DNA dC->dU-editing enzyme APOBEC-3G
OS = Homo sapiens OX = 9606 GN = APOBEC3G PE = 1 SV = 1
MKPHFRNTVERMYRDTFSYNFYNRPILSRRNTVWLCYEVKTKGPSRPPLDAKI
FRGQVYSELKYHPEVIRFFHWFSKWRKLHRDQEYEVTWYISWSPCTKCTRDIM
ATFLAEDPKVTLTIFVARLYYFWDPDYQEALRSLCQKRDGPRATMKIMNYDEFQ
HCWSKFVYSQRELFEPWNNLPKYYILLHIMLGEILRHSMDPPTFTFNFNNEPWV
RGRHETYLCYEVERMHNDTWVLLNQRRGFLCNQAPHKHGFLEGRHAELCFLD
VIPFWKLDLDQDYRVTCFTSWSPCFSCAQEMAKFISKNKHVSLCIFTARIYDDQ
GRCQEGLRTLAEAGAKISIIMTYSEFKHCWIDTFVDHQGCPFQPWDGLDEHSQD
LSGRLRAILQNQEN
SEQ ID NO: 95
>sp|Q6NTF7|ABC3H_HUMAN DNA dC->dU-editing enzyme APOBEC-3H
OS = Homo sapiens OX = 9606 GN = APOBEC3H PE = 1 SV = 4
MALLTAETFRLQFNNKRRLRRPYYPRKALLCYQLTPQNGSTPTRGYFENKKKC
HAEICFINEIKSMGLDETQCYQVTCYLTINSPCSSCAWELVDFIKAHDHLNLGIFA
SRLYYHWCKPQQKGLRLLCGSQVPVEVMGFPEFADCWENFVDHEKPLSFNPY
KMLEELDKNSRAIKRRLERIKIPGVRAQGRYMDILCDAEV
Petromyzon marinus cytosine deaminase (pmCDA1), Genbank ABO15149.1
SEQ ID NO: 96
MTDAEYVRIHEKLDIYTFKKQFFNNKKSVSHRCYVLFELKRRGERRACFWGYA
VNKPQSGTERGIHAEIFSIRKVEEYLRDNPGQFTINWYSSWSPCADCAEKILEW
YNQELRGNGHTLKIWACKLYYEKNARNQIGLVVNLRDNGVGLNVMVSEHYQCC
RKIFIQSSHNQLNENRWLEKTLKRAEKRRSELSIMIQVKILHTTKSPAV
Petromyzon marinus cytosine deaminase (pmCDA1) R187W, as used in
Target-AID, SEQ ID NO: 97
MTDAEYVRIHEKLDIYTFKKQFFNNKKSVSHRCYVLFELKRRGERRACFWGYA
VNKPQSGTERGIHAEIFSIRKVEEYLRDNPGQFTINWYSSWSPCADCAEKILEW
YNQELRGNGHTLKIWACKLYYEKNARNQIGLWNLRDNGVGLNVMVSEHYQCC
RKIFIQSSHNQLNENRWLEKTLKRAEKWRSELSIMIQVKILHTTKSPAV
E. coli TadA, SEQ ID NO: 98
MSEVEFSHEYWMRHALTLAKRAWDEREVPVGAVLVHNNRVIGEGWNRPIGRH
DPTAHAEIMALRQGGLVMQNYRLIDATLYVTLEPCVMCAGAMIHSRIGRVVFGA
RDAKTGAAGSLMDVLHHPGMNHRVEITEGILADECAALLSDFFRMRRQEIKAQK
KAQSSTD
S. aureus TadA, SEQ ID NO: 99
MTNDIYFMTLAIEEAKKAAQLGEVPIGAIITKDDEVIARAHNLRETLQQPTAHAEHI
AIERAAKVLGSWRLEGCTLYVTLEPCVMCAGTIVMSRIPRVVYGADDPKGGCS
GSLMNLLQQSNFNHRAIVDKGVLKEACSTLLTTFFKNLRANKKSTN
S. pyogenes TadA, SEQ ID NO: 100
MPYSLEEQTYFIMEALKEAEKSLQKAEIPIGCVIVKDGEIIGRGHNAREESNQAI
MHAEMMAINEANAHEGNWRLLDTTLFVTIEPCVMCSGAIGLARIPHVIYGASNQ
KFGGADSLYQILTDERLNHRVQVERGLLAADCANIMQTFFRQGRERKKIAKHLIK
EQSDPFD
S. typhi TadA, SEQ ID NO: 101
MSDVELDHEYWMRHALTLAKRAWDEREVPVGAVLVHNHRVIGEGWNRPIGRH
DPTAHAEIMALRQGGLVLQNYRLLDTTLYVTLEPCVMCAGAMVHSRIGRVVFG
ARDAKTGAAGSLIDVLHHPGMNHRVEIIEGVLRDECATLLSDFFRMRRQEIKALK
KADRAEGAGPAV
A. aeolicus TadA, SEQ ID NO: 102
MGKEYFLKVALREAKRAFEKGEVPVGAIIVKEGEIISKAHNSVEELKDPTAHAEM
LAIKEACRRLNTKYLEGCELYVTLEPCIMCSYALVLSRIEKVIFSALDKKHGGVVS
VFNILDEPTLNHRVKWEYYPLEEASELLSEFFKKLRNNII
S. pombe TAD2, SEQ ID NO: 103
MAGDSVKSAIIGIAGGPFSGKTQLCEQLLERLKSSAPSTFSKLIHLTSFLYPNSVD
RYALSSYDIEAFKKVLSLISQGAEKICLPDGSCIKLPVDQNRIILIEGYYLLLPELLP
YYTSKIFVYEDADTRLERCVLQRVKAEKGDLTKVLNDFVTLSKPAYDSSIHPTRE
NADIILPQKENIDTALLFVSQHLQDILAEMNKTSSSNTVKYDTQHETYMKLAHEIL
NLGPYFVIQPRSPGSCVFVYKGEVIGRGFNETNCSLSGIRHAELIAIEKILEHYPA
SVFKETTLYVTVEPCLMCAAALKQLHIKAVYFGCGNDRFGGCGSVFSINKDQSI
DPSYPVYPGLFYSEAVMLMREFYVQENVKAPVPQSKKQRVLKREVKSLDLSRF
K
S. cerevisiae TAD1, SEQ ID NO: 104
MVSCQGTRPCIVNLLTMPSEDKLGEEISTRVINEYSKLKSACRPIIRPSGIREWTI
LAGVAAINRIDGGANKIEILSIATGVKALPDSELQRSEGKILHDCHAEILALRGANT
VLLNRIQNYNPSSGDKFIQHNDEIPARFNLKENWELALYISRLPCGDASIVISFLND
NCKNDDFIKIEDSDEFQYVDRSVKTILRGRLNFNRRNVVRTKPGRYDSNITLSKS
CSDKLLMKQRSSVLNCLNYELFEKPVFLKYIVIPNLEDETKHHLEQSFHTRLPNL
DNEIKFLNCLKPFYDDKLDEEDVPGLMCSVKLFMDDFSTEEAILNGVRNGFYTK
SSKPLRKHCQSQVSRFAQWELFKKIRPEYEGISYLEFKSRQKKRSQUIAIKNILS
PDGWIPTRTDDVK
S. cerevisiae TAD2, SEQ ID NO: 105
MOHIKHMRTAVRLARYALDHDETPVACIFVHTPTGQVMAYGMNDTNKSLTGVA
HAEFMGIDQIKAMLGSRGVVDVFKDITLYVTVEPCIMCASALKQLDIGKVVFGCG
NERFGGNGTVLSVNHDTCTLVPKNNSAAGYESIPGILRKEAIMLLRYFYVRQNE
RAPKPRSKSDRVLDKNTFPPMEWSKYLNEEAFIETFGDDYRTCFANKVDLSSN
SVDWDLIDSHQDNIIQELEEQCKMFKFNVHKKSKV
A. thaliana TAD2, SEQ ID NO: 106
MEEDHCEDSHNYMGFALHQAKLALEALEVPVGCVFLEDGKVIASGRNRTNETR
NATRHAEMEAIDQLVGQWQKDGLSPSQVAEKFSKCVLYVTCEPCIMCASALSF
LGIKEVYYGCPNDKFGGCGSILSLHLGSEEAQRGKGYKCIRGGIMAEEAVSLFK
CFYEQGNPNAPKPHRPVVQRERT
X. laevis ADAT2, SEQ ID NO: 107
MEPLQITEEIQNWMHKAFQMAQDALNNGEVPVGCLMVYGNQVVGKGRNEVN
ETKNATQHAEMVAIDQVLDWCEMNSKKSTDVFENIVLYVTVEPCIMCAGALRLL
KIPLVVYGCRNERFGGCGSVLNVSGDDIPDTGTKFKCIGGYQAEKAIELLKTFYK
QENPNAPKSKVRKKE
X. tropicalis ADAT2, SEQ ID NO: 108
WITEEIQNWMHKAFQMAQDALNNGEVPVGCLAWYDNQVVGKGRNEVNETKNA
TRHAEMVAIDQVLDWCEKNSKKSRDVFENIVLYVTVEPCIMCAGALRLLKIPLVV
YGCRNERFGGCGSVLNVAGDNIPDTGTEFKYIGGYQAEKAVELLKTFYKQENP
NAPRSKVRKKE
D. rerio ADAT2, SEQ ID NO: 109
MQEVGVDPEKNDFLQPSDSEVQTWMAKAFDMAVEALENGEVPVGCLMVYNN
EIIGKGRNEVNETKNATRHAEMVALDQVLDWCRLREKDCKEVCEQTVLYVTVE
PCIMCAAALRLLRIPFVVYGCKNERFGGCGSVLDVSSDHLPHTGTSFKCIAGYR
AEEAVEMLKTFYKQENPNAPKPKVRKDSINPQDGAAVIQVMRGPPDEETETIAH
LS
B. Taurus ADAT2, SEQ ID NO: 110
MEAKAGPTAATDGAYSVSAEETEKWMEQAMQMAKDALDNTEVPVGCLMVYN
NEVVGKGRNEVNQTKNATRHAEMVAIDQALDWCRRRGRSPSEVFEHTVLYVT
VEPCIMCAAALRLMRIPLVVYGCQNERFGGCGSVLDIASADLPSTGKPFQCTPG
YRAEEAVEMLKTFYKQENPNAPKSKVRKKECHKS
M. musculus ADAT2, SEQ ID NO: 111
MEEKVESTTTPDGPCVVSVQETEKWMEEAMRMAKEALENIEVPVGCLMVYNN
EVVGKGRNEVNQTKNATRHAEMVAIDQVLDWCHQHGQSPSTVFEHTVLYVTV
EPCIMCAAALRLMKIPLVVYGCQNERFGGCGSVLNIASADLPNTGRPFQCIPGY
RAEEAVELLKTFYKQENPNAPKSKVRKKDCQKS
H. sapiens ADAT2 SEQ ID NO: 112
MEAKAAPKPAASGACSVSAEETEKVVMEEAMHMAKEALENTEVPVGCLMVYNN
EVVGKGRNEVNQTKNATRHAEMVAIDQVLDWCRQSGKSPSEVFEHTVLYVTV
EPCIMCAAALRLMKIPLVVYGCQNERFGGCGSVLNIASADLPNTGRPFQCIPGY
RAEEAVEMLKTFYKQENPNAPKSKVRKKECQKS
BE1 for Mammalian expression (rAPOBEC1-XTEN-dCas9.NLS)
SEQ ID NO: 113
MSSETGPVAVDPTLRRRIEPHEFEVFFDPRELRKETCLLYEINWGGRHSIWRHT
SQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSN
EAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPS
KKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGY
IDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETIT
PWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNF
MQLIHDDSLTFKEDEQKAQVSGQGDSLHEHIANLAGSPAIKKGELQTVKWDELV
KVMGRHKPENIVIEMARENQTTQKGQKNSRERNAKRIEEGIKELGSQLKEHPVE
NTQLQNEKLYLYYLQNGRDMWDQELDENIRLSDYDVDAIVPQSFLKDDSIDNKV
LTRSDKNRGKSDNVPSEEVVKKMKNYWRQLLNAKLITQRKFDNLTKAERGGLS
ELDKAGFEKROLVETRQITKHVAQILDSRMNTKYDENDKLIREVKVITLKSKINSD
FRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLEETNGETGEIVWD
KGRDFATVRKVLSMPQVNIVKKTEVQTGGFSKESILPKRNSDKLIARKKDWDPK
KYGGFDSPTVAYSVLVVAKVEKGKSKKLKSVKELLGETIMERSSFEKNPIDFLEA
KGYKEVKKDLIIKLPKYSLFELENGRKRMLASAGELQKGNELALPSKYVNFLYLA
SHYEKLKGSPEDNEQKQLFVEQHKHYLDEHEQISEFSKRVILADANLDKVLSAY
NKHRDKPIREQAENIIHLFTLTNLGAPAAFKYFDTTIDRKRYTSTKEVLDATLIHQ
SITGLYETRIDLSQLGGDSGGSPKKKRKV
BE2 (rAPOBEC1-XTEN-dCas9-UGI-NLS) SEQ ID NO: 114
MSSETGPVAVDPURRRIEPHEFEVFFDPRELRKETCLLYEINWGGRHSIWRHT
SQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSN
EAHWPRYPHLWVRLYVLELYCIILGLPPCLNILRRKQPQLTFFTIALQSCHYQRL
PPHILWATGLKSGSETPGTSESATPESDKKYSIGLAIGTNSVGWAVITDEYKVPS
KKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLR
KKLVDSTDKADLRLIYLALAHMIKFRGHFLIEGDLNPONSDVDKLFIQLVQTYNQL
FEENPINASGVDAKAILSARLSKSRRLENLIAQLPGEKKNGLFGNLIALSLGLTPN
FKSNFDLAEDAKLQLSKDTYDDDLDNILLAQIGDQYADLFLAAKNLSDAILLSDILR
VNTEITKAPLSASMIKRYDEHHQDLTLLKALVRQQLPEKYKEIFFDQSKNGYAGY
IDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETIT
PWNFEEVVDKGASAQSFIERMTNFDKNLPNEKVLPKHSLLYEYFTVYNELTKVK
YVTEGVIRKPAFLSGEOKKANDLLFKTNRKVTVKQLKEDYFKKIECFDSVEISGV
EDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELV
KVMGRHKPENIVIEMARENQTTQKGQKNSRERIMKRIEEGIKELGSQILKEHPVE
NTQLQNEKLYLYYLQNGRDMWDQELDINRLSDYDVDAIVPQSFLKDDSIDNKV
LTRSDKNRGKSDNVPSEEVVKKMKNYWRQLLNAKLITQRKFDNLTKAERGGLS
ELDKAGFIKRQLVETRQITKHVAQILDSRVINTKYDENDKLIREVKVITLKSKLVSD
FRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLA
SHYEKLKGSPEDNECKQLFVEQHKHYLDEIIEQISEFSKRVILADANLDKVLSAY
NKHRDKPIREQAENIIHLFTLTNLGAPAAFKYFDTTIDRKRYTSTKEVLDATLIHQ
SITGLYETRIDLSQLGGDSGGSTNLSDIIEKETGKQLVIQESILMLPEEVEEVIGNK
PESDILVHTAYDESTDENVIMLLTSDAPEYKPWALVEQDSNGENKIKMLSGGSPK
KKRKV
BE3 (rAPOBEC1-XTEN-Cas9n-UGI-NLS) SEQ ID NO: 115
MSSETGPVAVDPTLRRRIEPHEFEVFFDPRELRKETCLLYEINWGGRHSIWRHT
SQNTNKHVEVNFIEKFTTERYFCPNTRCSITWFLSWSPCGECSRAITEFLSRYP
HVTLFIYIARLYHHADPRNRQGLRDLISSGVTIQINATEQESGYCWRNFVNYSPSN
EAHWPRYPHLWVRLYVLELYCHLGLPPOLNELRRKQPQLTFFTIALQSCHYQRL
PPHILWATGLKSGSETPGTSESATPESDKKYSIGLAIGTNSVGWAVITDEYKVPS
KKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFICIVQTYNCI
FEENPINASGVDAKAILSARLSKSRRLENLIAQLPGEKKNGLFGNLIALSLGLTPN
FKSNFDLAEDAKLQLSKIDTYDDDLDNLLAQIGDQYADLFLAAKNLSDAILLSDILR
VNTEITKAPLSASMIKRYDEHHQDLTLLKALVRQQLPEKYKEIFFDQSKNGYAGY
IDGGASQEEFYKFIKPILEKMDGTEELLVKLNREDLLRKQRTFDNGSIPHQHLGE
LHAILRRQEDFYPFLKDNREKIEKILTFRIPYYVGPLARGNSRFAWMTRKSEETIT
PWNFEEVVDKGASAQSFIERMTNFDKNLPNEKVLPKHSLLYEYFTVYNELTKVK
YVTEGIVIRKPAFLSGEQKKANDLIFKTNRKVTVKQLKEDYFKKIECFDSVEISGV
EDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVE
NTQLONEKLYLYYLQNGRDMYVDQELDINRLSOYDVDHIVPQSFLKDDSIDNKV
LTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWD
KGRDFATVRKVLSMPOVNIVKKTEVQTGGFSKESILPKRNSDKLIARKKDWDPK
KYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLA
SHYEKLKGSPEDNEQKQLFVEQHKHYLDEHEQISEFSKRVILADANLDKVLSAY
NKHRDKPIREQAENIIHLFTLTNLGAPAAFKYFDTTIDRKRYTSTKEVLDATLIHQ
SITGLYETRIDLSQLGGDSGGSTNLSDIIEKETGKQLVIQESILIMLPEEVEEVIGNK
PESDILVHTAYDESTDENVMLITSDAPEYKPWALVIQDSNGENKIKMLSGGSPK
KKRKV
CDA1-BE3: SEQ ID NO: 116
MTDAEYVRIHEKLDIYTFKKOFFNNKKSVSHRCYVLFELKRRGERRACFWGYA
VNKPQSGTERGIHAEIFSIRKVEEYLRDNPGQFTINWYSSWSPCADCAEKILEW
YNQELRGNGHTLKIWACKLYYEKNARNQIGLWNLRDNGVGLNVMVSEHYQCC
RKIFIQSSHNQLNENRWLEKTLKRAEKRRSELSIMIQVKILHTTKSPAVSGSETP
GTSESATPESDKKYSIGLAIGTNSVGWAVITDEYKVPSKKFKVLGNTDRHSIKKN
LIGALLEDSGETAEATRLKRTARRRYTRRKNRICYWEIFSNEMAKVDDSFFHRL
EESFLVEEDKKHERHPIFGNIVDEVAYHEKYPTIYHLRKKLVDSTDKADLRLIYLA
LAHMEKFRGHFLIEGDLNPQNSDVDKLFIQLVQTYNQLFEENPINASGVDAKAILS
ARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGE
QKKANDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRR
RYTGWGRLSRKLINGIRDKQSGKTILDFLKSDGFANRNFMQLIHDDSLTFKEDIQ
KAQVSGQGDSLHEHIANLAGSPAIKKGILQTVKWDELVKVMGRHKPENIVIEMA
RENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYF
FYSNIMNFFKTEITLANGEIRKRPL
IETNGETGEIVWDKGRDFATVRKVLSMPQVNIVKKTEVQTGGFSKESILPKRNS
DKLIARKKDWDPKKYGGFIDSPTVAYSVLWAKVEKGKSKKLKSWELLGITIMER
SSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYTS
TKEVLDATLIHQSITGLYETRIDLSQLGGDSGGSTNLSDIIEKETGKOLVIQESILM
LPEEVEEVIGNKPESDILVHTAYDESTDENVMLLTSDAPEYKPWALVIQDSNGE
NKIKMLSGGSPKKKRKV
AID-BE3: SEQ ID NO: 117
MDSLLMNRRKFLYQFKNVRWAKGRRETYLCYVVKRRDSATSFSLDFGYLRNK
NGCHVELLFLRYISDWDLDPGRCYRVTWFTSWSPCYDCARHVADFLRGNPNL
SLRIFTARLYFCEDRKAEPEGLRRLHRAGVQIAIMTFKDYFYCWNTFVENHERT
FKAWEGLHENSVRLSRQLRRILLPLYEVDDLRDAFRTLGLSGSETPGTSESATP
ESDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVIDAKAI
LSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIK
PILEKMDGTEELLVKLNREDLLRKORTFDNGSIPHQIHLGELHAILRRQEDFYPFL
KDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQ
NGRDMYVDQELIDINRLSDYDVDHIVPQSFLKDDSIDNKVLTRSDKNRGKSDNV
PSEDNKKNAKNYWRQLLNAKLETQRKFDNLTKAERGGLSELDKAGFIKRQLVET
RQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATA
KYFFYSNIMNFFKTEETLANGEERKRPLEETNGETGEIVWDKGRDFATVRKVLSNA
PQVNIVKKTENVQTGGFSKESILPKRNSDKLEARKKDWDPKKYGGFDSPTVAYSV
LWAKVEKGKSKKLKSVIKELLGITIMERSSFEKNPIDFLEAKGYKEVKKDLIEKLPK
YSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPEREQAENIE
HLFTLTNLGAPAAFKYFDTTEDRKRYTSTKEVLDATLIHQSITGLYETREDLSQLG
GDSGGSTNLSDIIEKETGKQLVIQESILMLPEEVEEVIGNKPESDILVHTAYDEST
DENVMLLTSDAPEYKPWALVIQDSNGENKIKNALSGGSPKKKRIKV
BE3-Gam: SEQ ID NO: 118
MAKPAKRIKSAAAAYVPQNRDAVITDIKRIGDWREASRLETEMNDAIAEITEKFA
ARIAPIKTDIETLSKGVQGWCEANRDELTNGGKVKTANLVTGDVSWRVRPPSV
SIRGMDAVMETLERLGLQRFIRTKQEINKEAILLEPKAVAGVAGITVKSGIEDFSII
PFEQEAGISGSETPGTSESATPESSSETGPVAVDPTLRRRIEPHEFEVFFDPRE
LRKETCLLYEINWGGRHSIWRHTSQNTNKHVEVNFIEKFTTERYFCPNTRCSIT
WFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPOLNI
LRRKQPQLTFFTIALQSCHYQRLPPHILWATGLKSGSETPGTSESATPESDKKY
SIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKL
NREDLLRKQRTFDNGSPHQIHLGELHAERRQEDFYPFLKDNREKIEKILTFRIP
YYVGPLARGNSRFAWMTRKSEETITPWNFEEVVDKGASAQSFIERMTNFDKNL
PNEKVLPKHSLLYEYFTVYNELTKVKYVTEGMRKPAFLSGEQKKANDLIFKTNR
KVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLIN
GIRDKQSGKTILDFLKSDGFANRNFMOLIHDDSLTFKEDIQKAQVSGQGDSLHE
HIANLAGSPAIKKGILQTVKVVDELVKVMGRHKPENIVIEMARENQTTQKGQKNS
RERMKRIEEGIKELGSQLKEHPVENTQLQNEKLYLYYLQNGRDIMYVDQELDIN
RLSDYDVDHIVPQSFLKDDSIDNKLTRSDKNRGKSDNVPSEEVVKKIMKNYWR
QLLNAKLITQRKFDNLTIKAERGGLSELDKAGFIKRQLVETRQITKHVAQILDSRM
NTKYDENDKLIREVKVITLKSKLVSDFRKDFQFYKVREINNYHHAHDAYLNAVVG
TALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGG
FSKESILPKRNSDKLIARKKDWDPKKYGGFDSPTVAYSVIANAKVEKGKSKKLK
SVKELLGITIMERSSFEKNPIDFLEAKGYKEVKKDLIIKLPKYSLFELENGRKRMLA
SAGELQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDSGGSTNLSDIEKET
GKQLVIQESILMLPEEVEEVIGNKPESDILVHTAYDESTDENVMLLTSDAPEYKP
WALVIQDSNGENKIKMLSGGSPKKKRKV
SaBE3-Gam: SEQ ID NO: 119
MAKPAKRIKSAAAAYVPQNRDAVITDIKRIGDLQREASRLETEMNDAIAEITEKFA
ARIAPIKTDIETLSKGVQGWCEANRDELTNGGKVKTANLVTGDVSWRVRPPSV
SIRGMDAVMETLERLGLQRFIRTKQEINKEAILLEPKAVAGVAGITVKSGIEDFSII
PFEQEAGISGSETPGTSESATPESSSETGPVAVDPTLRRRIEPHEFEVFFDPRE
LRKETCLLYEINWGGRHSIWRHTSQNTNKHVEVNFIEKFTTERYFCPNTRCSIT
WFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNI
LRRKQPQLTFFTIALQSCHYQRLPPHILWATGLKSGSETPGTSESATPESGKRN
YILGLAIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAK
RRGVHNVNEVEEDTGNELSTKEQISRNSKALEEKYVAELQLERLKKDGEVRGSI
NRFKTSDYVKEAKQLLKVQKAYHQLDQSFIDTYIDLLETRRTYYEGPGEGSPFG
WKDIKEWYEMLMGHCTYFPEELRSVKYAYNADLYNALNDLNNLVITRDENEKL
EYYEKFQIIENVFKQKKKPTLKQIAKEILVNEEDIKGYRVTSTGKPEFTNLKVYHDI
KDITARKEIIENAELLDQIAKILTIYQSSEDIQEELTNLNSELTQEEIEQISNLKGYTG
THNLSLKAINLILDELWHTNDNQIAIFNRLKLVPKKVDLSQQKEIPTTLVDDFILSP
VVKRSFIQSIKVINAIIKKYGLPNDIIIELAREKNSKDAQKMINEMQKRNRQTNERI
EEIIRTTGKENAKYLIEKIKLHDMQEGKCLYSLEAIPLEDLLNNPFNYEVDHIIPRS
VSFDNSFNNKVLVKQEENSKKGNRTPFQYLSSSDSKISYETFKKHILNLAKGKG
RISKTKKEYLLEERDINRFSVQKDFINRNLVDTRYATRGLMNLLRSYFRVNNLDV
KVKSINGGFTSFLRRKWKFKKERNKGYKHHAEDALIIANADFIFKEWKKLDKAKK
VMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQT
YQKLKLIMEQYGDEKNPLYKYYEETGNYLTKYSKKDNGPVIKKIKYYGNKLNAHL
DITDDYPNSRNKVVKLSLKPYRFDVYLDNGVYKFVTVKNLDVIKKENYYEVNSK
CYEEAKKLKKISNQAEFIASFYNNDLIKINGELYRVIGVNNDLLNRIEVNMIDITYR
EYLENMNDKRPPRIIKTIASKTQSIKKYSTDILGNLYEVKSKKHPQIIKKGGSPKKK
RKVSSDYKDHDGDYKDHDIDYKDDDDKSGGSTNLSDIIEKETGKQLVIQESILML
PEEVEEVIGNKPESDILVHTAYDESTDENVMLLT
SDAPEYKPWALVIQDSNGENKIKMLSGGSPKKKRKV
BE4: SEQ ID NO: 120
MSSETGPVAVDPTLRRRIEPHEFEVFFDPRELRKETCLLYEINWGGRHSIWRHT
SQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSN
EAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAI
GTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNRED
LLRKQRTFDNGSIPHQIHLGELHAILRRQEDFYPFLKDNREKIEKILTFRIPYYVGP
L
ARGNSRFAWMTRKSEETITPWNFEEVVDKGASAQSFIERMTNFDKNLPNEKVL
PKHSLLYEYFTVYNELTKVKYVTEGMRKPAFLSGEQKKANDLLFKTNRKVTVK
QLKEDYFKKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLINGIRDK
QSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYD
VDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVUGGFSKESIL
PKRNSDKLIARKKDWDPKKYGGFDSPTVAYSVLVVAKVEKGKSKKLKSWELL
GITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTID
RKRYTSTKEVLDATLIHQSITGLYETRIDLSQLGGDSGGSGGSGGSTNLSDIIEK
ETGKQLVIQESILMLPEEVEEVIGNKPESDILVHTAYDESTDENVMLLTSDAPEY
KPWALVIQDSNGENKIKMLSGGSGGSGGSTNLSDIIEKETGKQLVIQESILMLPE
EVEEVIGNKPESDILVHTAYDESTDENVMLLTSDAPEYKPWALVIQDSNGENKIK
MLSGGSPKKKRK
BE4-Gam: SEQ ID NO: 121
MAKPAKRIKSAAAAYVPQNRDAVITDIKRIGDWREASRLETEMNDAIAEITEKFA
ARIAPIKTDIETLSKGVQGWCEANRDELTNGGKVKTANLVTGDVSWRVRPPSV
SIRGMDAVMETLERLGLQRFIRTKQEINKEAILLEPKAVAGVAGITVKSGIEDFSII
PFEQEAGISGSETPGTSESATPESSSETGPVAVDPTLRRRIEPHEFEVFFDPRE
LRKETCLLYEINWGGRHSIWRHTSQNTNKHVEVNFIEKFTTERYFCPNTRCSIT
WFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNI
LRRKQPQLTFFTIALQSCHYQRLPPHILWATGLKSGGSSGGSSGSETPGTSESA
TPESSGGSSGGSDKKYSIGLAIGTNSVGWAVITDEYKVPSKKFKVLGNTDRHSI
KKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFI
KPILEKMDGTEELLVKLNREDLLRKQRTFDNGSIPHQHLGELHAILRRQEDFYP
FLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFL
SGEQKKANDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVNAKQLK
RRRYTGWGRLSRKLINGIRDKQSGKTELDFLKSDGFANRNFMQLIH
DDSLTFKEDIQKACWSGQGDSLHEHIANLAGSPAIKKGILQTVKVVDELVKVIMG
RHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKD
FQFYKVREINNYHHAHDAYLNAVVGTALIKKYPKLESEFWGDYKVYDVRKMIA
KSEQEIGKATAKYFFYSNIMNFFKTEITLANGEIRKRPLIETNGETGEIVWDKGRD
FATVRKVLSNAPQVNIVKKTEVQTGGFSKESILPKRNSDKLIARKKDWDPKKYGG
FDSPTVAYSVLANAKVEKGKSKKLKSVKELLGITIMERSSFEKNPIDFLEAKGYK
EVKKDLIIKLPKYSLFELENGRKRNALASAGELQKGNELALPSKYVNFLYLASHYE
KLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGL
YETRIDLSQLGGDSGGSGGSGGSTNLSDHEKETGKQLVIQESILMLPEEVEEVI
GNKPESDILVHTAYDESTDENVMLLTSDAPEYKPWALVIQDSNGENKIKVILSGG
SGGSGGSTNLSDHEKETGKQLVIQESILMLPEEVEEVIGNKPESDILVHTAYDES
TDENVMLLTSDAPEYKPWALVIQDSNGENKIKMLSGGSPKKKRK
SaBE4: SEQ ID NO: 122
MSSETGPVAVDPURRRIEPHEFEVFFDPRELRKETCLLYEINWGGRHSIWRHT
SQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSN
EAHWPRYPHLWVRLYVLELYCHLGLPPOLNILRRKOPQLTFFTIALQSCHYQRL
PPHILWATGLKSGGSSGGSSGSETPGTSESATPESSGGSSGGSGKRNYILGLA
IGITSVGYGIIDYETRDVIDAGVRLFKEANVENNEGRRSKRGARRLKRRRRHRIQ
RVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKT
SDYVKEAKQLLKVQKAYHQLDQSFIDTYIDLLETRRTYYEGPGEGSPFGWKDIK
EWYEMLMGHCTYFPEELRSVKYAYNADLYNALNDLNNLVITRDENEKLEYYEK
FQIIENVFKQKKKPTLKQIAKEILVNEEDIKGYRVTSTGKPEFTNLKVYHDIKDITA
RKEHENAELLDQIAKILTIYQSSEDIQEELTNLNSELTQEEIEQISNLKGYTGTHNL
SLKAINLILDELWHTNDNQIAIFNRLKLVPKKVDLSQQKEIPTTLVDDFILSPWKR
SFIQSIKVINAIIKKYGLPNDIIIELAREKNSKDAQKMINEMQKRNRQTNERIEEHRT
TGKENAKYLIEKIKLHDMEGKOLYSLEAIPLEDLLNNPFNYEVDHIIPRSVSFDN
SFNNKVLVKQEENSKKGNRTPFQYLSSSDSKISYETFKKHILNLAKGKGRISKTK
KEYLLEERDINRFSVQKDFINRNLVDTRYATRGLMNLLRSYFRVNNLDVKVKSIN
GGFTSFLRRKWKFKKERNKGYKHHAEDALIIANADFIFKEWKKLDKAKKVMENQ
MFEEKOAESMPEIETEQEYKEIFITPHQIKHIKDFKDYKYSHRVDKKPNRELINDT
LYSTRKDDKGNTLIVNNLNGLYDKDNDKLKKLINKSPEKLLMYHHDPQTYQKLK
LIMEQYGDEKNPLYKYYEETGNYLTKYSKKDNGPVIKKIKYYGNKLNAHLDITDD
YPNSRNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDITYREYLEN
VINDKRPPRIIKTIASKTQSIKKYSTDILGNLYEVKSKKHPQIIKKGGSPKKKRKVS
SDYKDHDGDYKDHDIDYKDDDDKSGGSGGSGGSTNLSDIIEKETGKQLVIQESI
LIMLPEEVEEVIGNKPESDILVHTAYDESTDENVNALLTSDAPEYKPWALVIQDSN
GEN
KIMILSGGSGGSGGSTNLSDIEEKETGKQLVIQESILMLPEEVEEVIGNKPESDIL
VHTAYDESTDENVMLLTSDAPEYKPWALVIQDSNGENKIKMLSGGSPKKKRKV
SaBE4-Gam: SEQ ID NO: 123
MAKPAKRIKSAAAAYVPQNRDAVITDIKRIGDLQREASRLETEMNDAIAEITEKFA
ARIAPIKTDIETLSKGVQGWCEANRDELTNGGKVKTANLVTGDVSWRVRPPSV
SIRGMDAVMETLERLGLQRFIRTKQEINKEAILLEPKAVAGVAGITVKSGIEDFSII
PFEQEAGISGSETPGTSESATPESSSETGPVAVDPTLRRRIEPHEFEVFFDPRE
LRKETCLLYEINWGGRHSIWRHTSQNTNKHVEVNFIEKFTTERYFCPNTRCSIT
WFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNI
LRRKQPQLTFFTIALQSCHYQRLPPHILWATGLKSGGSSGGSSGSETPGTSESA
TPESSGGSSGGSGKRNYILGLAIGITSVGYGIIDYETRDVIDAGVRLFKEANVEN
NEGRRSKRGARRLKRRRRHRIQRVKKLLFDYNLLTDHSELSGINPYEARVKGLS
QKLSEEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTYIDLLE
TRRTYYEGPGEGSPFGWKDIKEWYEMLMGHCTYFPEELRSVKYAYNADLYNA
LNDLNNLVITRDENEKLEYYEKFQIIENVFKQKKKPTLKQIAKEILVNEEDIKGYRV
TSTGKPEFTNLKVYHDIKDITARKEIIENAELLDQIAKILTIYQSSEDIQEELTNLNS
ELTQEEIEQISNLKGYTGTHNLSLKAINLILDELWHTNDNQIAIFNRLKLVPKKVDL
SQQKEIPTTLVDDFILSPVVKRSFIQSIKVINAIIKKYGLPNDIIIELAREKNSKDAQK
MINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTRYATRGL
MNLLRSYFRVNNLDVKVKSINGGFTSFLRRKWKFKKERNKGYKHHAEDALIIAN
ADFIFKEWKKLDKAKKVMENQMFEEKQAESMPEIETEQEYKEIFITPHQIKHIKD
FKDYKYSHRVDKKPNRELINDTLYSTRKDDKGNTLIVNNLNGLYDKDNDKLKKLI
NKSPEKLLMYHHDPQTYQKLKLIMEQYGDEKNPLYKYYEETGNYLTKYSKKDN
GPVIKKIKYYGNKLNAHLDITDDYPNSRNKVVKLSLKPYRFDVYLDNGVYKFVTV
KNLDVIKKENYYEVNSKCYEEAKKLKKISNQAEFIASFYNNDLIKINGELYRVIGV
NNDLLNRIEVNMIDITYREYLENMNDKRPPRIIKTIASKTOSIKKYSTDILGNLYEV
KSKKHPQIIKKGGSPKKKRKVSSDYKDHDGDYKDHDIDYKDDDDKSGGSGGSG
GSTNLSDIIEKETGKQLVIQESILMLPEEVEEVIGNKPESDILVHTAYDESTDENV
MLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVI
QDSNGENKIKMLSGGSPKKKRKV
BE4max and AncSE4max, SEQ ID NO: 124
MKRTADGSEFESPKKKRKVSGGSSGGSSGSETPGTSESATPESSGGSSGGS
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKIEKI
LTFREPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKANDLL
FKTNRKVTVKQLKEDYFKKIECFDSVEISGVEDRFNASLGTYHDLLKIIKDKDFLD
NEENEDELEDIVLTLTLFEDREMIEERLKTYAHLFDDKVMKQLKRRRYTGWGRL
SRKLINGIRDKQSGKTILDFLKSDGFANRNFMLIHDDSLTFKEDIQKAQVSGQG
DSLHEHIANLAGSPAIKKGILQTVKWDELVKVMGRHKPENIVIEMARENQTTQK
GQKNSRERMKREEEGIKELGSQILKEHPVENTQLQNEKLYLYYLQNGRDMWD
QELDINRLSDYDVDHIVPQSFLKDDSIDNKVLTRSDKNRGKSDNVPSEENNKKM
KNYWRQLLNAKLITQRKFDNLTKAERGGLSELDKAGFIKRQLVETRQITKHVAQI
LDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENG
RKRMLASAGELQKGNELALPSKYVNFLYLASHYEKLKGSPEDNEQKQLFVEQH
KHYLDEHEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSGGSGG
SGGSTNLSDIIEKETGKQLVIQESILMLPEEVEEVIGNKPESDILVHTAYDESTDE
NVNALLTSDAPEYKPWALVIQDSNGENKIKMLSGGSGGSGGSTNLSDHEKETGK
QLVIQESILMLPEEVEEVIGNKPESDILVHTAYDESTDENVMLLTSDAPEYKPWA
LVIQDSNGENKIKMLSGGSKRTADGSEFEPKKKRKV
Rat APOBEC1, SEQ ID NO: 125
SSETGPVAVDPTLRRRIEPHEFEVFFDPRELRKETCLLYEINWGGRHSIWRHTS
QNTNKHVEVNFIEKFTTERYFCPNTRCSITWFLSWSPCGECSRAITEFLSRYPH
VTLFIYIARLYHHADPRNRQGLRDLISSGVTIQIMTEQESGYDNRNFVNYSPSNE
AHWPRYPHLWVRLYVLELYCHLGLPPOLNILRRKQPQLTFFTIALQSCHYQRLP
PHILWATGLK
Anc689 APOBEC, SEQ ID NO: 126
SSETGPVAVDPTLRRRIEPHEFEVFFDPRELRKETCLLYEIKWGTSHKIWRHSS
KNTTKHVEVNFIEKFTSERHFCPSTSCSITWFLSWSPCGECSKAITEFLSQHPN
VTLVIYVARLYHHVIDQQNRQGLRDLVNSGVTIQIMTAPEYDYCWRNFVNYPPG
KEAHWPRYPPLWMKLYALELHAGILGLPPCLNILRRKOPQLTFFTIALQSCHYQ
RLPPHILWATGLK
Anc687 APOBEC, SEQ ID NO: 127
SSETGPVAVDPTLRRRIEPHEFEVFFDPRELRKEACLLYEIKWGTSHKIWRNSG
KNTTKHVEVNFIEKFTSERHFCPSISCSITWFLSWSPCWECSKAIREFLSQHPN
VTLVIYVARLFQHMDCMRQGLRDLVNSGVTIQIMTASEYDHCWRNFVNYPPG
KEAHWPRYPPLWMKLYALELHAGILGLPPCLNILRRKQPQLTFFTIALQSCHYQ
RLPPHILWATGLK
Anc686 APOBEC, SEQ ID NO: 128
SSETGPVAVDPTLRRRIEPEFFNRNYDPRELRKETYLLYEIKWGKESKIWRHTS
NNRTQHAEVNFLENFFNELYFNPSTHCSITWFLSWSPCGECSKAIVEFLKEHPN
VNLEIYVARLYLCEDERNRQGLRDLVNSGVTIRIMNLPDYNYCWRTFVSHQGG
DEDYWPRHFAPWVRLYVLELYCIILGLPPCLNILRRKQPQLTFFTIALQSCHYQR
LPPHILWATGLK
Anc655 APOBEC, SEQ ID NO: 129
SSETGPVAVDPTLRRRIEPFYFQFNNDPRACRRKTYLCYELKQDGSTINVWKRT
LHNKGRHAEICFLEKISSLEKLDPAQHYRITWYMSWSPCSNCAQKIVDFLKEHP
HVNLRIYVARLYYHEEERYQEGLRNLRRSGVSIRVMDLPDFEHCWETFVDNGG
GPFQPWPGLEELNSKQLSRRLQAGILGLPPCLNILRRKQPQLTFFTIALQSCHY
QRLPPHILWATGLK
Anc733 APOBEC, SEQ ID NO: 130
SSETGPVAVDPTLRRRIEPFHFQFNNDPRAYRRKTYLOYELKQDGSTWVLDRT
LRNKGRHAEICFLDKINSWERLDPAQHYRVTWYMSWSPCSNCAQQVVDFLKE
HPHVNLRIFAARLYYHEQRRYQEGLRSLRGSGVPVAVMTLPDFEHOWETFVDH
GGRPFQPWDGLEELNSRSLSRRLQAGILGLPPCLNILRRKQPQLTFFTIALQSC
HYQRLPPHILWATGLK
APOBEC ancestor #686, SEQ ID NO: 131
EFFNRNYDPRELRKETYLLYEIKWGKESKIWRHWCTSNNRTQHAEVNFLENFF
NELYFNPSTHCSITWFLSWSPCGECSKAIVEFLKEHPNVNLEIYVARLYLCEDER
NRQGLRDLVNSGVTIRIMNLPDYNYCWRTFVSHQGGDEDYWPRHFAPWVRL
APOBEC ancestor #733, SEQ ID NO: 132
FHFQFNNDPRAYRRKTYLCYELKQDGSTWVLDRGCTLRNKGRHAEICFLDKIN
SWERLDPAQHYRVTWYMSWSPCSNCAQQVVDFLKEHPHVNLRIFAARLYYHE
QRRYQEGLRSLRGSGVPVAVMTLPDFEHOWETFVDHGGRPFQPWDGLEELN
SRSLSRRLQAG
APOBEC ancestor #656, FERNY, SEQ ID NO: 133
FERNYDPRELRKETYLLYEIKWGKSGKLWRHWCQNNRTQHAEVYFLENIFNAR
RFNPSTHCSITWYLSWSPCAECSQKIVDFLKEHPNVNLEIYVARLYYHEDERNR
QGLRDLVNSGVTIRIMDLPDYNYCWKTFVSDQGGDEDYWPGHFAPWIKQYSL
KL
APOBEC ancestor #655, SEQ ID NO: 134
FYFQFNNDPRACRRKTYLCYELKQDGSTWVWKRGCTLHNKGRHAEICFLEKIS
SLEKLDPAQHYRITWYMSWSPCSNCAQKIVDFLKEHPHVNLRIWARLYYHEEE
RYQEGLRNLRRSGVSIRVIMDLPDFEHCWETFVDNGGGPFQPWPGLEENSKQL
SRRLQAG
APOBEC ancestor #649, SEQ ID NO: 135
FYEEFNNTLKSCRHKTLLCFSLKQDENTTLWKWGYAHNNGRHAEILVLREIENY
EKLDPAAKYRITLYMSYSPCNDCADKIVDFLKKHPNVNLNIKVSRLYYHEDEKYQ
EGLRNLKQPGVSLKVMDRSDFEECFDLFVDPGGGEFQPWPGLEEKSKQYSAT
WAG
ABE6.3, SED ID: 136
MSEVEFSHEYWNARHALTLAKRAWDEREVPVGAVLVHNNRVIGEGWNRPIGRH
DPTAHAEIMALRQGGLVIMNYRLIDATYVTLEPCVNACAGAMIHSRIGRVVFGAR
DAKTGAAGSLNADVLHHPGMNHRVEITEGILADECAALLSDFFRNARRQEIKAOKK
AQSSTDSGGSSGGSSGSETPGTSESATPESSGGSSGGSSEVEFSHEYWMRH
ALTLAKRAWDEREVPVGAVINLNNRVIGEGWNRSEGLHDPTAHAEIMALRQGG
LVMQNYRLIDATLYVTFEPCVMCAGAMIHSRIGRVVFGVRNAKTGAAGSLNADVL
HYPGMNHRVEITEGILADECAALLCYFFRVIRRQVFNAQKKAQSSTDSGGSSGG
SSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYID
GGASQEEFYKFIKPILEKVIDGTEELLVKLNREDLLRKQRTFDNGSPHQIHLGEL
HAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKY
VTEGMRKPAFLSGEQKKANDLIFKTNRKVTVKQLKEDYFKKIECFDSVEISGVE
DRFNASLGTYHDLLKIIKDKDFLDNEENEDILEDIVLTLTLFEDREMIEERLKTYAH
LFDDKVMKQLKRRRYTGWGRLSRKLINGIRDKOSGKTILDFLKSDGFANRNFM
QLIHDDSLTFKEDIQKAQVSGQGDSLHEHANLAGSPAIKKGILQTVKVVDELVK
VMGRHKPENIVIEMARENQTTQKGOKNSRERMKRIEEGIKELGSCAKEHPVEN
TQLQNEKLYLYYLQNGRDMYVDQELDINRLSDYDVDHIVPQSFLKDDSIDNKVL
TRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKINSOF
RKDFQFYKVREINNYHHAHDAYLNAVVGTALIKKYPKLESEFVYGDYKVYDVRK
MIAKSEQEIGKATAKYFFYSNIVINFFKTEITLANGEIRKRPLIETNGETGERANDK
GRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAK
GYKEVKKDLIIKLPKYSLFELENGRKRMLASAGELQKGNELALPSKYVNFLYLAS
HYEKLKGSPEDNECKQLFVEQHKHYLDEIIEQISEFSKRVILADANLDKVLSAYN
KHRDKPIREQAENIIHLFTLTNLGAPAAFKYFDTTIDRKRYTSTKEVLDATLIHQSI
TGLYETRIDLSQLGGDSGGSPKKKRKV
ABE7.8, SED ID: 137
MSEVEFSHEYWMRHALTLAKRAWDEREVPVGAVLVHNNRVIGEGWNRPIGRH
DPTAHAEIMALRQGGLVMONYRLIDATLYVTLEPCVMCAGAMIHSRIGRVVFGA
RDAKTGAAGSLMDVLHHPGMNHRVEITEGILADECAALLSDFFRMRRQEIKAQK
KAQSSTDSGGSSGGSSGSETPGTSESATPESSGGSSGGSSEVEFSHEYWNIR
HALTLAKRALDEREVPVGAVLVLANRVIGEGWNRAIGLHDPTAHAEIMALRQGG
LVMQNYRLIDATLYVTFEPCVMCAGAMIHSRIGRVVFGVRNAKTGAAGSLMDVL
HYPGMNHRVEITEGILADECNALLCYFFRMRRQVFNAQKKAQSSTDSGGSSGG
SSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYED
GGASQEEFYITIKPILEKMDGTEELLVKLNREDLLRKQRTFDNGSPHQEHLGEL
HAELRRQEDFYPFLKDNREKIEKELTFRIPYYVGPLARGNSRFAWMTRKSEETITP
WNFEEVVDKGASAQSFIERMTNFDKNLPNEKVLPKHSLLYEYFTVYNELTKVKY
VTEGNARKPAFLSGEQKKANDLIFKTNRIKVTVKQLKEDYFKKIECFDSVEISGVE
DRFNASLGTYHDLLKIIKDKDFLDNEENEDILEDIVLTLTLFEDREMIEERLKTYAH
LFDDKVMKQLKRRRYTGWGRLSRKLINGIRDIKQSGKTILDFLKSDGFANRNFM
QLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVEN
TQLQNEKLYLYYLQNGRDMYVDQELDINIRLSDYDVDHIVPQSFLIKDDSIDNKVL
TRSDKNRGIKSDNVPSEEVVKKMKNYWRQLLNAKLITQRKFDNLTKAERGGLSE
LDKAGFIKRQLVETRQITKHVAQILDSRMNTIKYDENDKLIREVIKVITLKSKLVSDF
RKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDK
GRDFATVRKVLSMPQVNIVKIKTEVQTGGFSKESILPKRNSDKLIARKKDWDPKK
YGGFDSPTVAYSVLWAKVEKGKSKKLKSVIKELLGITIMERSSFEKNPIDFLEAK
GYKEVKKDLIIKLPKYSLFELENGRKRMLASAGELQKGNELALPSKYVNFLYLAS
HYEKLKGSPEDNEQKQLFVEQHKHYLDEHEQISEFSKRVILADANLDKVLSAYN
KHRDKPIREQAENIIHLFTLTNLGAPAAFKYFDTTIDRKRYTSTKEVLDATLIHQSI
TGLYETRIDLSQLGGDSGGSPKKKRKV
ABE7.9, SED ID: 138
MSEVEFSHEYWMRHALTLAKRAWDEREVPVGAVLVHNNRVIGEGWNRPIGRH
DPTAHAEIMALRQGGLVMQNYRLIDATLYVTLEPCVMCAGAMIHSRIGRVVFGA
RDAKTGAAGSLMDVLHHPGMNHRVEITEGILADECAALLSDFFRMRRQEIKAQK
KAQSSTDSGGSSGGSSGSETPGTSESATPESSGGSSGGSSEVEFSHEYWMR
HALTLAKRALDEREVPVGAVLVLANRVIGEGWNRAIGLHDPTAHAEIMALRQGG
LVMQNYRLIDATLYVTFEPCVMCAGAMIHSRIGRVVFGVRNAKTGAAGSLMDVL
HYPGMNHRVEITEGILADECNALLCYFFRMPRQVFNAQKKAQSSTDSGGSSGG
SSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKK
LVDSTDKADLRLIYLALAHMIKFRGHFLIEGDLNPQNSDVDKLFIQLVQTYNQLFE
ENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYID
GGASQEEFYKFIKPILEKVIDGTEELLVKLNREDLLRKQRTFDNGSPHQIFILGEL
HAILRRQEDFYPFLKDNREKIEKILTFRIPYYVGPLARGNSRFAWMTRKSEETITP
WNFEEVVDKGASAQSFIERNATNFDKNLPNEKVLPKHSLLYEYFTVYNELTKVKY
VTEGMRKPAFLSGEOKKANDLLFKTNRKVTVKQLKEDYFKKIECFDSVEISGVE
DRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFM
QLIHDDSLTFKEDEQKAQVSGQGDSLHEHIANLAGSPAIKKGELQTVKVVDELVK
VMGRHKPENIVIEMARENUTQKGQKNSRERNAKRIEEGEKELGSQLKEHPVEN
TQLQNEKLYLYYLQNGRDIMYVDQELDINIRLSDYDVDHIVPQSFLKDDSIDNKVL
TRSDKNRGKSDNVPSEEVVKKMKNYVVRQLLNAKLETQRKFDNLTKAERGGLSE
LDKAGFEKROLVETRQETKHVAQILDSRMNTKYDENDKLIREVKVITLKSKLVSDF
RKDFQFYKVREINNYHHAHDAYLNAVVGTALIKKYPKLESEFVYGDYKVYDVRK
MIAKSEQEIGKATAKYFFYSNIMNFFKTEETLANGEERKRPLIETNGETGEIVWDK
GRDFATVRKVLSMPQVNIVKIKTEVQTGGFSKESILPKRNSDKLIARKKDWDPKK
YGGFDSPTVAYSVLWAKVEKGKSIKKLKSVKELLGITIMERSSFEKNPIDFLEAK
GYKEVKKDLIIKLPKYSLFELENGRKRMLASAGELQKGNELALPSKYVNFLYLAS
HYEKLKGSPEDNEQKQLFVEQHKHYLDEHEQISEFSKRVILADANLDKVLSAYN
KHRDKPIREQAENIIHLFTLTNLGAPAAFKYFDTTIDRKRYTSTKEVLDATLIFIQSI
TGLYETRIDLSQLGGDSGGSPIKKIKRKV
ABE7.10, SEQ ID: 139
MSEVEFSHEYWMRHALTLAKRAWDEREVPVGAVLVHNNRVIGEGWNRPIGRH
DPTAHAEIMALRQGGLVMQNYRLIDATLYVTLEPCVMCAGAMIHSRIGRVVFGA
RDAKTGAAGSLNADVLHHPGMNHRVEITEGILADECAALLSDFFRNARRQEIKAQK
KAQSSTDSGGSSGGSSGSETPGTSESATPESSGGSSGGSSEVEFSHEYWNAR
HALTLAKRARDEREVPVGAVLVLANRVIGEGWNRAIGLHDPTAHAEIMALRQG
GLVMQNYRLIDATLYVTFEPCVMCAGAMIHSRIGRVVFGVRNAKTGAAGSLMD
VLHYPGVINHRVEITEGILADECAALLCYFFRIMPRQVFNAQKKAQSSTDSGGSS
GGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLT
PNFKSNFDLAEDAKLQLSKOTYDDDLDNLLAQIGDQYADLFLAAKNLSDAILLSDI
LRVNTEITKAPLSASMIKRYDEHHQDLTLLKALVRQQLPEKYKEIFFDQSKNGYA
GYIDGGASQEEFYKFIKPILEKVIDGTEELLVKLNREDLLRKQRTFDNGSIPHQIHL
GELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTK
VKYVTEGMRKPAFLSGEQKKANDLIFKTNRKVTVKQLKEDYFKKIECEDSVEIS
GVEDRFNASLGTYHDLLKIIKDKDFLDNEENEDILEDIVLTLTLFEDREMIEERLKT
YAHLFDDKVIMKQLKRRRYTGWGRLSRKLINGIRDKQSGKTILDFLKSDGFANRN
FMQLIHDDSLTFKEDICKAQVSGQGDSLHEHIANLAGSPAIKKGILQTVKVVDEL
VKVMGRHKPENIVIEMARENQTTOKGQKNSRERIMKRIEEGIKELGSQILKEHPV
ENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDV
RKIMIAKSEQEIGKATAKYFFYSNIMNFFKTEITLANGEIRKRPLIETNGETGEIVW
DKGRDFATVRKVLSIMPQVNIVKKTEVQTGGFSKESILPKRNSDKLIARKKDWDP
KKYGGFDSPTVAYSVLVVAKVEKGKSKKLKSVKELLGITEMERSSFEKNPIDFLE
AKGYKEVKKDLIIKLPKYSLFELENGRKRNALASAGELQKGNELALPSKYVNFLYL
ASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIH
QSITGLYETRIDLSQLGGDSGGSPKKKRKV
SPACE, SEQ ID NO: 140
MKRTADGSEFESPKKKRKVSEVEFSHEYWNARHALTLAKRARDEREVPVGAVL
VLNNRVIGEGWNRAIGLHDPTAHAEIMALRQGGLVMQNYRLIDATLYGTFEPCV
MCAGAMIHSRIGRVVFGVRNAKTGAAGSLMDVLHYPGMNHRVEITEGILADEC
AALLCYFFRMPRQVFNAQKKAQSSTDSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPQNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKANDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGR
DMYVDQELDINIRLSQYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKENVDPKKYGGFDSPTVAYSVLVVA
KVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSG
GSKRTADGSEFEPKKKRKVSGGSSGGSSGSETPGTSESATPESSGGSSGGST
DAEYVRIHEKLDIYTFKKQFFNNKKSVSHRCYVLFELKRRGERRACFWGYAVN
KPQSGTERGIHAEIFSIRKVEEYLRDNPGQFTINWYSSWSPCADCAEKILEWYN
QELRGNGHTLKIWACKLYYEKNARNQIGLWNLRDNGVGLNVMVSEHYQCCRKI
FIQSSHNQLNENRWLEKTLKRAEKWRSELSIMIQVKILHTTKSPAVSGGSGGSG
GSTNLSDIIEKETGKQLVIQESILMLPEEVEEVIGNKPESDILVHTAYDESTDENV
WILLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVI
QDSNGENKIKNALGSGATNFSLLKQAGDVEENPGPMVSKGEELFTGVVPILVEL
DGDVNGHKFSVSGEGEGDATYGKLTLKFICTTGKLPVPWPTLVTTLTYGVQCF
SRYPDHMKOHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLA
DHYQQNTPIGDGPVLLPDNHYLSTQSALSKDPNEKRDHMVLLEFVTAAGITLGNA
DELYKSGGSPKKKRK
SPACEΔUGI, SEQ ID NO: 141
MKRTADGSEFESPKKKRKVSEVEFSHEYWNARHALTLAKRARDEREVPVGAVL
VLNNRVIGEGWNRAIGLHDPTAHAEIMALRQGGLVMQNYRLIDATLYGTFEPCV
MCAGAMIHSRIGRVVFGVRNAKTGAAGSLIMDVLHYPGMNHRVEITEGILADEC
AALLCYFFRMPRQVFNAQKKAQSSTDSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKANDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGR
DMYVDQELDINIRLSDYDVDHIVPQSFLKDDSIDNKVLTRSDKNRCKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQL
FVEQHKHYLDEHEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSG
GSKRTADGSEFEPKKKRKVSGGSSGGSSGSETPGTSESATPESSGGSSGGST
DAEYVRIHEKLDIYTFKKQFFNNKKSVSHRCYVLFELKRRGERRACFWGYAVN
KPQSGTERGIHAEIFSIRKVEEYLRDNPGQFTINWYSSWSPCADCAEKILEWYN
QELRGNGHTLKIWACKLYYEKNARNQIGLWNLRDNGVGLNVMVSEHYQCCRKI
FIQSSHNQLNENRWLEKTLKRAEKWRSELSIMIQVKILHTTKSPAVGSGATNFSL
LKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAIMPEGY
VQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYL
STQSALSKDPNEKRDHMVLLEFVTAAGITLGNADELYKSGGSPKKKRKV
SPACE-NG, SEQ ID NO: 142
MKRTADGSEFESPKKKRKVSEVEFSHEYWNARHALTLAKRARDEREVPVGAVL
VLNNRVIGEGWNRAIGLHDPTAHAEIMALRQGGLVMQNYRLIDATLYGTFEPCV
MCAGAMIHSRIGRVVFGVRNAKTGAAGSLIMDVLHYPGMNHRVEITEGILADEC
AALLCYFFRMPRQVFNAQKKAQSSTDSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKP1LEK
MDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKANDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFNAQLIHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESIRPKRNSDKLIARKKDWDPKKYGGFVSPTVAYSVLVVA
KVEKGKSKKLKSVKELLGITIMERSSFEKNPIDFLEAKGYKEVKKDLIIKLPKYSLF
ELENGRKRMLASARFLQKGNELALPSKYVNFLYLASHYEKLKGSPEDNEQKQL
FVEQHKHYLDENEQISEFSKRVILADANLDKVLSAYNKHRDKPIREQAENIIHLFT
LTNLGAPRAFKYFDTTIDRKVYRSTKEVLDATLIHQSITGLYETRIDLSQLGGDSG
GSKRTADGSEFEPKKKRKVSGGSSGGSSGSETPGTSESATPESSGGSSGGST
DAEYVRIHEKLDIYTFKKOFFNNKKSVSHRCYVLFELKRRGERRACFWGYAVN
KPQSGTERGIHAEIFSIRKVEEYLRDNPGQFTINWYSSWSPCADCAEKILEWYN
QELRGNGHTLKIWACKLYYEKNARNQIGLWNLRDNGVGLNVMVSEHYQCCRKI
FIQSSHNQLNENRWLEKTLKRAEKWRSELSIMIQVKILHTTKSPAVSGGSGGSG
GSTNLSDIIEKETGKQLVIQESILMLPEEVEEVIGNKPESDILVHTAYDESTDENV
WILLTSDAPEYKPWALVIQDSNGENKIKMLSGGSGGSGGSTNLSDHEKETGKQL
VIQESILMLPEEVEEVIGNKPESDILVHTAYDESTDENVMLLTSDAPEYKPWALVI
QDSNGENKIKMLGSGATNFSLLKQAGDVEENPGPMVSKGEELFTGVVPILVEL
DGDVNGHKFSVSGEGEGDATYGKLTLKFICTTGKLPVPWPTLVTTLTYGVQCF
SRYPDHIMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLA
DHYQQNTPIGDGPVLLPDNHYLSTQSALSKDPNEKRDHMULLEFVTAAGITLGIM
DELYKSGGSPKKKRKV
SPACE-VRQR, SEQ ID NO: 143
MKRTADGSEFESPKKKRKVSEVEFSHEYWNARHALTLAKRARDEREVPVGAVL
VLNNRVIGEGWNRAIGLHDPTAHAEIMALRQGGLVMQNYRLIDATLYGTFEPCV
MCAGAMIHSRIGRVVFGVRNAKTGAAGSLIMDVLHYPGMNHRVEITEGILADEC
AALLCYFFRMPRQVFNAQKKAQSSTDSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKANDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFNAQLIHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQ1LKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKDWDPKKYGGFVSPTVAYSVLVVA
KVEKGKSKKLKSVKELLGITIMERSSFEKNPIDFLEAKGYKEVKKDLIIKLPKYSLF
ELENGRKRMLASARELQKGNELALPSKYVNFLYLASHYEKLKGSPEDNEQKQL
FVEQHKHYLDENEQISEFSKRVILADANLDKVLSAYNKHRDKPIREQAENIIHLFT
LTNLGAPAAFKYFDTTIDRKQYRSTKEVLDATLIHQSITGLYETRIDLSQLGGDSG
GSKRTADGSEFEPKKKRKVSGGSSGGSSGSETPGTSESATPESSGGSSGGST
DAEYVRIHEKLDIYTFKKQFFNNKKSVSHRCYVLFELKRRGERRACFWGYAVN
KPQSGTERGIHAEIFSIRKVEEYLRDNPGQFTINWYSSWSPCADCAEKILEWYN
QELRGNGHTLKIWACKLYYEKNARNQIGLWNLRDNGVGLNVMVSEHYQCCRKI
FIQSSHNQLNENRWLEKTLKRAEKWRSELSIMIQVKILHTTKSPAVSGGSGGSG
GSTNLSDIIEKETGKQLVIQESILMLPEEVEEVIGNKPESDILVHTAYDESTDENV
WILLTSDAPEYKPWALVIQDSNGENKIKMLSGGSGGSGGSTNLSDHEKETGKQL
VIQESILMLPEEVEEVIGNKPESDILVHTAYDESTDENVMLLTSDAPEYKPWALVI
QDSNGENK1KMLGSGATNFSLLKQAGDVEENPGPMVSKGEELFTGVVPILVEL
DGDVNGHKFSVSGEGEGDATYGKLTLKFICTTGKLPVPWPTLVTTLTYGVQCF
SRYPDHIMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLA
DHYQQNTPIGDGPVLLPDNHYLSTQSALSKDPNEKRDHMVLLEFVTAAGITLGM
DELYKSGGSPKKKRKV
SPACE-NAA, SEQ ID NO: 144
MKRTADGSEFESPKKKRKVSEVEFSHEYWNARHALTLAKRARDEREVPVGAVL
VLNNRVIGEGWNRAIGLHDPTAHAEIMALRQGGLVMQNYRLIDATLYGTFEPCV
MCAGAMIHSRIGRVVFGVRNAKTGAAGSLIMDVLHYPGMNHRVEITEGILADEC
AALLCYFFRMPRQVFNAQKKAQSSTDSGGSSGGSSGSETPGTSESATPESSG
GSSGGSDKKYSIGLAIGTNSVGWAVITDEYKVPSKKFKVLGNTDRHSEKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLAREDLLRKQRTFDNGSIPHQIHLGELHAILRRQEDFYPFLKDN
REKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKANDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFNAQUHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEIQTVGQNGGLFDDNPKSPLEVTPSKLVPLKKELNPKKYGGYQKPTTAY
PVLLITDTKQLIPISVMNKKQFEQNPVKFLRDRGYQQVGKNDFIKLPKYTLVDIG
DGIKRLWASSKEIHKGNQLVVSKKSOILLYHAHHLDSDLSNDYLQNHNQQFDVL
FNEMSFSKKOKLGKEHIQKIENVYSNKKNSASIEELAESFIKLLGFTQLGATSPFN
FLGVKLNQKQYKGKKDYILPCTEGTLIRQSITGLYETRVDLSKIGEDSGGSKRTA
DGSEFEPKKKRKVSGGSSGGSSGSETPGTSESATPESSGGSSGGSTDAEYVR
IHEKLDIYTFKKQFFNNKKSVSHRCYVLFELKRRGERRACFWGYAVNKPQSGT
ERGIHAEIFSIRKVEEYLRDNPGQFTINWYSSWSPCADCAEKILEVVYNQELRGN
GHTLKIWACKLYYEKNARNQIGLWNLRDNGVGLNVMVSEHYQCORKIFIQSSH
NQLNENRWLEKTLKRAEKWRSELSIMIQVKILHTTKSPAVSGGSGGSGGSTNL
SDIIEKETGKQLVIQESILIMLPEEVEEVIGNKPESDILVHTAYDESTDENVMLLTS
DAPEYKPWALVIQDSNGENKIKMLSGGSGGSGGSTNLSDHEKETGKQLVIQESI
LMLPEEVEEVIGNKPESDILVHTAYDESTDENVMLLTSDAPEYKPWALVIQDSN
GENKIKMLGSGATNFSLLKQAGDVEENPGPMVSKGEELFTGVVPILVELDGDV
NGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGITLGMDELY
KSGGSPKKKRKV
TadA*(ABE8e), SEQ ID NO: 145
SEVEFSHEYWNARHALTLAKRARDEREVPVGAVLVLNNRVEGEGWNRAIGLHDP
TAHAEIMALRQGGLVMQNYRLIDATLYVTFEPCVMCAGAMIHSRIGRVVFGVRN
SKRGAAGSLANVLNYPGWINHRVEITEGILADECAALLCDFYRMPRQVFNAQKK
AQSSIN
TadA*(ABE8e_V82G), SEQ ID NO: 146
SEVEFSHEYWNARHALTLAKRARDEREVPVGAVLVLNNRVEGEGWNRAIGLHDP
TAHAEIMALRQGGLVMQNYRLIDATLYGTFEPCVMCAGAMIHSREGRVVFGVRN
SKRGAAGSLMNVLNYPGMNHRVEITEGILADECAALLCDFYRMPRQVFNAQKK
AQSSIN
TadA*(ABE8e_K20A/R21A), SEQ ID NO: 147
SEVEFSHEYWMRHALTLAAAARDEREVPVGAVLVLNNRVIGEGWNRAIGLHDP
TAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKK
AQSSIN
TadA* (ABE8.8), SEQ ID NO: 148
SEVEFSHEYWMRHALTLAKRARDEREVPVGAVLVLNNRVIGEGWNRAIGLHDP
TAHAEIMALRQGGLVMQNYRLIDATLYVTFEPCVMCAGAMIHSRIGRVVFGVRN
AKTGAAGSLMDVLHHPGMNHRVEITEGILADECAALLORFFRMPRRVFNAQKK
AQSSTD
TadA* (ABE8.13), SEQ ID NO: 149
SEVEFSHEYWMRHALTLAKRARDEREVPVGAVLVLNNRVIGEGWNRAIGLHOP
TAHAEIMALRQGGLVMQNYRLYDATLYVTFEPCVMCAGAMIHSRIGRVVFGVR
NAKTGAAGSLADVLHHPGVINHRVEITEGILADECAALLORFFRMPRRVFNAQK
KAQSSTD
TadA* (ABE8.17), SEQ ID NO: 150
SEVEFSHEYWMRHALTLAKRARDEREVPVGAVLVLNNRVIGEGWNRAIGLHOP
TAHAEIMALRQGGLVMQNYRLIDATLYSTFEPCVVICAGAMIHSRIGRVVFGVRN
AKTGAAGSLMDVLHYPGMNHRVEITEGILADECAALLCYFFRMPRRVFNAQKK
AQSSTD
TadA* (ABE8.20), SEQ ID NO: 151
SEVEFSHEYWIMRHALTLAKRARDEREVPVGAVLVLNNRVIGEGWNRAIGLHDP
TAHAEIMALRQGGLVMQNYRLYDATLYSTFEPCVMCAGAMIHSRIGRVVFGVR
NAKTGAAGSLMDVLHHPGMNHRVEITEGILADECAALLCRFFRMPRRVFNAQK
KAQSSTD
rAPOBEC1(FERNY), SEQ ID NO: 152
FERNYDPRELRKETYLLYEIKWGKSGKLWRHWCQNNRTQHAEVYFLENIFNAR
RFNPSTHCSITWYLSWSPCAECSQKIVDFLKEHPNVNLEIYVARLYYHEDERNR
QGLRDLVNSGVTIRIMDLPDYNYCWKTFVSDQGGDEDYWPGHFAPWIKQYSL
KL
rAPOBEC1(evoFERNY), SEQ ID NO: 153
FERNYDPRELRKETYLLYEIKWGKSGKLWRHWCQNNRTQHAEVYFLENIFNAR
RFNPSTHCSITWYLSWSPCAECSQKEVDFLKEHPNVNLEEYVARLYYPENERNR
QGLRDLVNSGVTIRIMDLPDYNYCWKTFVSDQGGDEDYWPGHFAPWIKQYSL
KL
rAPOBEC1(YE1), SEQ ID NO: 154
SSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPENRQGLRDLISSGVTIQIMTEQESGYCWRNFVNYSPSNEA
HWPRYPHLWVRLYVLELYCHLGLPPOLNILRRKQPQLTFFTIALQSCHYQRLPP
HILWATGLK
rAPOBEC1(YE2), SEQ ID NO: 155
SSETGPVAVDPTLRRRIEPHEFEVFFDPRELRKETCLLYEINWGGRHSIWRHTS
QNTNKHVEVNFIEKFTTERYFCPNTRCSITWFLSYSPCGECSRAITEFLSRYPHV
TLFIYIARLYHHADPRNRQGLEDLISSGVTIQIMTEQESGYCWRNFVNYSPSNEA
HWPRYPHLWVRLYVLELYCHLGLPPOLNILRRKQPQLTFFTIALQSCHYQRLPP
HILWATGLK
rAPOBEC1(YE3), SEQ ID NO: 156
SSETGPVAVDPTLRRR1EPHEFEVFFDPRELRKETCLLYEINWGGRHSIWRHTS
ONITNKHVEVNFIEKFTTERYFCPNTRCSITWFLSYSPCGECSRAITEFLSRYPHV
TLFIYIARLYHHADPENROGLEDLISSGVTIQIMTEQESGYCWRNFVNYSPSNEA
HWPRYPHLWVRLYVLELYCHLGLPPOLNILRRKQPQLTFFTIALQSCHYQRLPP
HILWATGLK
SpRY Cas9 variant, SEQ ID NO: 157
DKKYSIGLAIGTNSVGWAVITDEYKVPSKKFKVLGNTDRHSIKKNLIGALLFDSG
ETAERTRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRG
HFLIEGDLNPQNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKANDLL
FKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRL
SRKLINGIRDKQSGKTILDFLKSDGFANRNFMLIHDDSLTFKEDIQKAQVSGQG
DSLHEHIANLAGSPAIKKGILQTVKWDELVKVMGRHKPENIVIEMARENQTTQK
GQKNSRERNAKRIEEGIKELGSQILKEHPVENTQLQNEKLYLYYLQNGRDMWD
QELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQI
LDSRNANTKYDENDKLIREVKVITLKSKLVSDFRKDFQFYKVREINNYHHAHDAYL
NAVVGTALIKKYPKLESEFVYGDYKVYDVRKIMIAKSEQEIGKATAKYFFYSNIMN
FFKTEITLANGEIRKRPLIETNGETGEIVWDKGRDFATVRKVLSMPQVNIVKKTE
VQTGGFSKESIRPKRNSDKLIARKKDWDPKKYGGFLWPTVAYSVIANAKVEKG
KSKKLKSVKELLGITIMERSSFEKNPIDFLEAKGYKEVKKDLIIKLPKYSLFELENG
RKRMLASAKQLQKGNELALPSKYVNFLYLASHYEKLKGSPEDNEQKQLFVEQH
KHYLDEIIEQISEFSKRVILADANLDKVLSAYNKHRDKPIREQAENIIHLFTLTRLG
APRAFKYFDTTIDPKQYRSTKEVLDATLIHQSITGLYETRIDLSQLGGD
SpG Cas9 variant, SEQ ID NO: 158
DKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRL
SRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQK
GQKNSRERMKRIEEGIKELGSQILKEHPVENTQLQNEKLYLYYLQNGRDMYVD
QELDINRLSQYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQI
LDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTE
VQTGGFSKESILPKRNSDKLIARKKDWDPKKYGGFLWPTVAYSVLVVAKVEKG
KSKKLKSVKELLGITIMERSSFEKNPIDFLEAKGYKEVKKDLIIKLPKYSLFELENG
RKRMLASAKQLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLG
APAAFKYFDTTIDRKQYRSTKEVLDATLIHQSITGLYETRIDLSQLGGD
pCMV-bpNLS-TadA*(ABE8e)-nCas9-bpNLS-pmCDA1(R187W)-UGI-UGI-P2A-
EGFP, SEQ ID NO: 159
MKRTADGSEFESPKKKRKVSEVEFSHEYWIMRHALTLAKRARDEREVPVGAVL
VIANRVIGEGWNRAIGLHDPTAHAEIMALRQGGLVMQNYRLIDATLYVTFEPCV
MCAGAIMIHSRIGRVVFGVRNSKRGAAGSLMNVLNYPGMNHRVEITEGILADEC
AALLCDFYRMPRQVFNAQKKAQSSINSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEETKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKELTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFNAQLIHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQ1LKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQL
FVEQHKHYLDENEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSG
GSKRTADGSEFEPKKKRKVSGGSSGGSSGSETPGTSESATPESSGGSSGGST
DAEYVRIHEKLDIYTFKKQFFNNKKSVSHRCYVLFELKRRGERRACFWGYAVN
KPOSGTERGIHAEIFSIRKVEEYLRDNPGQFTINWYSSWSPCADCAEKILEWYN
QELRGNGHTLKIWACKLYYEKNARNQIGLWNLRDNGVGLNVMVSEHYQCCRKI
FIQSSHNQLNENRWLEKTLKRAEKWRSELSIMIQVKILHTTKSPAVSGGSGGSG
GSTNLSDHEKETGKQLVIQESILMLPEEVEEVIGNKPESDILVHTAYDESTDENV
MLLTSDAPEYKPWALVIQDSNGENKIKMLSGGSGGSGGSTNLSDIIEKETGKQL
VIQESILMLPEEVEEVIGNKPESDILVHTAYDESTDENVMLLTSDAPEYKPWALVI
QDSNGENKIKMLGSGATNFSLLKQAGDVEENPGPMVSKGEELFTGVVPILVEL
DGDVNGHKFSVSGEGEGDATYGKLTLKFICTTGKLPVPWPTLVTTLTYGVQCF
SRYPDHIMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLA
DHYQQNTPIGDGPVLLPDNHYLSTQSALSKDPNEKRDHMVLLEFVTAAGITLGIM
DELYKSGGSPKKKRKV
pCMV-bpNLS-TadA*(ABE8e)-nCas9-bpNLS-pmCDA1(R187W)-P2A-EGFP,
SEQ ID NO: 160
MKRTADGSEFESPKKKRKVSEVEFSHEYWIMRHALTLAKRARDEREVPVGAVL
VLNNRVIGEGWNRAIGLHDPTAHAEIMALRQGGLVMQNYRLIDATLYVTFEPCV
MCAGAIMIHSRIGRVVFGVRNSKRGAAGSLMNVLNYPGMNHRVEITEGILADEC
AALLCDFYRMPRQVFNAQKKAQSSINSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRECYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLIKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKELTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRY
TGWGRLSRKLINGIRDKQSGKTILDFLKSDGFANRNFNAQUHDDSLTFKEDIQKA
QVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQL
FVEQHKHYLDENEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSG
GSKRTADGSEFEPKKKRKVSGGSSGGSSGSETPGTSESATPESSGGSSGGST
DAEYVRIHEKLDIYTFKKQFFNNKKSVSHRCYVLFELKRRGERRACFWGYAVN
KPOSGTERGIHAEIFSIRKVEEYLRDNPGQFTINWYSSWSPCADCAEKILEWYN
QELRGNGHTLKIWACKLYYEKNARNQIGLWNLRDNGVGLNVMVSEHYQCCRKI
FIQSSHNQLNENRWLEKTLKRAEKWRSELSIMIQVKILHTTKSPAVGSGATNFSL
LKQAGDVEENPGPMVSKGEELFTGVVPILVELDGDVNGHKFSVSGEGEGDATY
GKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNS
HNVYIMADKOKNGIKVNFKIRHNIEDGSVQLADHYQQNTPIGDGPVLLPDNHYL
STOSALSKDPNEKRDHMVLLEFVTAAGITLGMDELYKSGGSPKKKRKV
pCMV-bpNLS-TadA*(ABE8e)-nCas9-bpNLS-hA3A-UGI-UGI-P2A-EGFP, SEQ
ID NO: 161
MKRTADGSEFESPKKKRKVSEVEFSHEYWIMRHALTLAKRARDEREVPVGAVL
VLNNRVIGEGWNRAIGLHDPTAHAEIMALROGGLVMQNYRLIDATLYVTFEPCV
MCAGAIMIHSRIGRVVFGVRNSKRGAAGSLMNVLNYPGMNHRVEITEGILADEC
AALLCDFYRMPRQVFNAQKKAQSSINSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDE
HHQDLTLIKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKELTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVIMKQLKRRRY
TGWGRLSRKLINGERDKQSGKTILDFLKSDGFANRNFMQLIHDDSLTFKEDIQKA
QVSGQGDSLHEHIANLAGSPAIKKGILQTVKVVDELVKVNAGRHKPENEVIEMARE
NOTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQL
FVEQHKHYLDEHEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSG
GSKRTADGSEFEPKKKRKVSGGSSGGSSGSETPGTSESATPESSGGSSGGSE
ASPASGPRHLMDPHIFTSNFNNGIGRHKTYLOYEVERLDNGTSVKMDQHRGFL
HNQAKNLLOGFYGRHAELRFLDLVPSLQLDPAQIYRVTWFISWSPCFSWGCAG
EVRAFWENTHVRLRIFAARIYDYDPLYKEALQMLRDAGAQVSIMTYDEFKHDAI
DTFVDHQGCPFQPWDGLDEHSOALSGRLRAILQNQGNSGGSGGSGGSTNLS
DIIEKETGKQLVIQESILMLPEEVEEVIGNKPESDILVHTAYDESTDENVMLLTSD
APEYKPWALVIQDSNGENKIKMLSGGSGGSGGSTNLSDIIEKETGKQLVIQESIL
MLPEEVEEVIGNKPESDILVHTAYDESTDENVMLLTSDAPEYKPWALVIQDSNG
ENKIKMLGSGATNFSLLKQAGDVEENPGPMVSKGEELFTGVVPILVELDGDVN
GHKFSVSGEGEGDATYGKLTLKFICTTGKLPVPWPTLVTTLTYGVQCFSRYPD
HMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQ
NTPIGDGPVLLPDNHYLSTQSALSKDPNEKRDHMVLLEFVTAAGITLGMDELYK
SGGSPKKKRKV
pCMV-bpNLS-TadA*(ABE8e)-nCas9-bpNLS-hA3A-P2A-EGFP, SEQ ID NO:
162
MKRTADGSEFESPKKKRKVSEVEFSHEYWIMRHALTLAKRARDEREVPVGAVL
VLNNRVIGEGWNRAIGLHDPTAHAEIMALROGGLVMQNYRLIDATLYVTFEPCV
MCAGAIMIHSRIGRVVFGVRNSKRGAAGSLMNVLNYPGMNHRVEITEGILADEC
AALLCDFYRMPRQVFNAQKKAQSSINSGGSSGGSSGSETPGTSESATPESSG
GSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEES
FLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARL
SKSRRLENLIAOLPGEKKNGLFGNLIALSLGLTPNFKSNFDLAEDAKLQLSKDTY
DODLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQ
KKANDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVIMKQLKRRRY
TGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARE
NOTTQKGQKNSRERMKRIEEGIKELGSQILKEHPVENTQLQNEKLYLYYLQNGR
DMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLF
ELENGRKRIVILASAGELQKGNELALPSKYVNFLYLASHYEKLKGSPEDNEQKQL
FVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGDSG
GSKRTADGSEFEPKKKRKVSGGSSGGSSGSETPGTSESATPESSGGSSGGSE
ASPASGPRHLMDPHIFTSNFNNGIGRHKTYLCYEVERLDNGTSVKMDQHRGFL
HNQAKNLLCGFYGRHAELRFLDLVPSLQLDPAQIYRVTWFISWSPCFSWGCAG
EVRAFLQENTHVRLRIFAARIYDYDPLYKEALQMLRDAGAQVSIMTYDEFKHCW
DTFVDHQGCPFQPWDGLDEHSQALSGRLRAILQNQGNGSGATNFSLLKQAGD
VEENPGPMVSKGEELFTGVVPILVELDGDVNGHKFSVSGEGEGDATYGKLTLK
FICTTGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHNVYIMA
DKQKNGIKVNFKIRHNIEDGSVOLADHYQQNTPIGDGPVLLPDNHYLSTQSALS
KDPNEKRDHMVLLEFVTAAGITLGMDELYKSGGSPKKKRKV
pCMV-bpNLS-hA3A-48AA-TadA*(ABE8e)-nCas9-bpNILS-UGI-UGI-P2A-
EGFP, SEQ ID NO: 163
MKRTADGSEFESPKKKRKVEASPASGPRHLMDPHIFTSNFNNGIGRHKTYLCY
EVERLDNGTSVKIMDQHRGFLHNQAKNLLCGFYGRHAELRFLDLVPSLQLDPA
QIYRVTWFISWSPCFSWGCAGEVRAFLQENTHVRLRIFAARIYDYDPLYKEALQ
MLRDAGAQVSIVITYDEFKHCWDTFVDHQGCPFQPWDGLDEHSQALSGRLRAI
LQNQGNSGSETPGTSESATPESSGGSSGGSSGSETPGTSESATPESSGGSSG
GSSEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGV
RNSKRGAAGSLIMNVLNYPGIMNHRVEITEGILADECAALLCDFYRMPRQVFNAQ
KKAQSSINSGGSSGGSSGSETPGTSESATPESSGGSSGGSDKKYSIGLAIGTN
SVGWAVITDEYKVPSKKFKVLGNTDRHSIKKNLIGALLFDSGETAEATRLKRTAR
RRYTRRKNRICYLQEIFSNEIMAKVDDSFFHRLEESFLVEEDKKHERHPIFGNIVD
EVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRK
QRTFDNGSIPHQHLGELHAILRRQEDFYPFLKDNREKIEKILTFRIPYYVGPLAR
GNSRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDV
DHIVPQSFLKDDSIDNKVLTRSDKNRCKSDNVPSEEVVKKMKNYWRQLLNAKLI
TQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIR
KRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQK
GNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDR
KRYTSTKEVLDATLIHQSITGLYETRIDLSQLGGDSGGSKRTADGSEFEPKKKRK
VSGGSGGSGGSTNLSDIIEKETGKQLVIQESILMLPEEVEEVIGNKPESDILVHTA
YDESTDENVMLLTSDAPEYKPWALVIQDSNGENKIKMLSGGSGGSGGSTNLSD
IIEKETGKOLVIQESILMLPEEVEEVIGNKPESDILVHTAYDESTDENVMLLTSDAP
EYKPWALVIQDSNGENKIKMLGSGATNFSLLKQAGDVEENPGPMVSKGEELFT
GVVPILVELDGDVNGHKFSVSGEGEGDATYGKLTLKFICTTGKLPVPAIPTLVTT
LTYGVQCFSRYPDHMKQHDFFKSAMPEGYVQERT1FFKDDGNYKTRAEVKFE
GDTLVNRIELKGIDFKEDGNILGHKLEYNYNSHNVYIMADKQKNGIKVNFKIRHNI
EDGSVQLADHYQQNTPIGDGPVLLPDNHYLSTQSALSKDPNEKRDHMVLLEFV
TAAGITLGMDELYKSGGSPKKKRKV
pCMV-bpNLS-hA3A-48AA-TadA*(ABE8e)-nCas9-bpNILS-P2A-EGFP, SEQ ID
NO: 164
MKRTADGSEFESPKKKRKVEASPASGPRHLMDPHIFTSNFNNGIGRHKTYLCY
EVERLDNGTSVKMDQHRGFLHNQAKNLLCGFYGRHAELRFLDLVPSLQLDPA
QIYRVTWFISWSPCFSWGCAGEVRAFLQENTHVRLRIFAARIYDYDPLYKEALQ
MLRDAGAQVSIMTYDEFKHCWDTFVDHQGCPFQPWDGLDEHSQALSGRLRAI
LQNQGNSGSETPGTSESATPESSGGSSGGSSGSETPGTSESATPESSGGSSG
GSSEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGV
RNSKRGAAGSLMNVLNYPGMNHRVEITEGILADECAALLCDFYRMPRQVFNAQ
KKAQSSINSGGSSGGSSGSETPGTSESATPESSGGSSGGSDKKYSIGLAIGTN
SVGWAVITDEYKVPSKKFKULGNTDRHSIKKNLIGALLFDSGETAEATRLKRTAR
RRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLF
LAAKNLSDAILLSDILRVNTEETKAPLSASMIKRYDEHHQDLTLLKALVRQQLPEK
YKEIFFDQSKNGYAGYIDGGASQEEFYKFIKPELEKMDGTEELLVKLNREDLLRK
QRTFDNGSIPHQHLGELHAILRRQEDFYPFLKDNREKIEKILTFRIPYYVGPLAR
GNSRFAWMTRKSEETITPWNFEEVVDKGASAQSFIERMTNFDKNLPNEKVLPK
HSLLYEYFTVYNELTKVKYVTEGMRKPAFLSGEQKKANDLLFKTNRKVTVKQL
KEDYFKKEECFDSVEISGVEDRFNASLGTYHDLLKIIKDKDFLDNEENEDILEDEVL
TLTLFEDREMIEERLKTYAHLFDDKVMKQLKRRRYTGWGRLSRKLINGERDKQS
GKTILDFLKSDGFANRNFMLIHDDSLTFKEDEQKAQVSGQGDSLHEHIANLAG
SPAIKKGILQTVKVVDELVKVNAGRHKPENIVIEMARENQTTQKGQKNSRERMKR
IEEGIKELGSQILKEHPVENTQLQNEKLYLYYLQNGRDMYVDQELDINRLSDYDV
DHIVPQSFLKDDSIDNKVLTRSDKNRCKSDNVPSEEVVKKMKNYWRQLLNAKLI
TQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYP
KLESEFVYGDYKVYDVRKIVIIAKSEQEIGKATAKYFFYSNIMNFFKTEITLANGEIR
KRPLIETNGETGEIVWDKGRDFATVRKVLSMPQVNIVKKTEVVGGFSKESILP
KRNSDKLIARKKDWDPKKYGGFDSPTVAYSVLVVAKVEKGKSKKLKSWELLGI
TIMERSSFEKNPIDFLEAKGYKEVKKDLIIKLPKYSLFELENGRKRMLASAGELQK
GNELALPSKYVNFLYLASHYEKLKGSPEDNEQKQLFVEQHKHYLDEHEQISEFS
KRVILADANLDKVLSAYNKHRDKPIREQAENIIHLFTLTNLGAPAAFKYFDTTIDR
KRYTSTKEVLDATLIHQS1TGLYETRIDLSQLGGDSGGSKRTADGSEFEPKKKRK
VGSGATNFSLLKQAGDVEENPGPIVIVSKGEELFTGVVPILVELDGDVNGHKFSV
SGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNIL
GHKLEYNYNSHNVYEMADKQKNGIKVNFKIRHNIEDGSVQLADHYQQNTPIGDG
PVLLPDNHYLSTQSALSKDPNEKRDHMVLLEFVTAAG1TLGMDELYKSGGSPKK
KRKV
pCMV-bpNLS-TadA*(ABE8e)-48AA-hA3A-nCas9-bpNLS-UGI-UGI-P2A-
EGFP, SEQ ID NO: 165
MKRTADGSEFESPKKKRKVSEVEFSHEYWMRHALTLAKRARDEREVPVGAVL
VLNNRVIGEGWNRAIGLHDPTAHAEIMALRQGGLVMQNYRLIDATLYVTFEPCV
MCAGAMIHSRIGRVVFGVRNSKRGAAGSLMNVLNYPGMNHRVEITEGILADEC
AALLCDFYRMPRQVFNAQKKAQSSINSGSETPGTSESATPESSGGSSGGSSG
SETPGTSESATPESSGGSSGGSEASPASGPRHLMDPHIFTSNFNNGIGRHKTY
LCYEVERLDNGTSVKMDCHRGFLHNQAKNLLQGFYGRHAELRFLDLVPSLQLD
PAQIYRVTWFISWSPCFSWGCAGEVRAFLQENTHVRLRIFAARIYDYDPLYKEA
LQMLRDAGAQVSIVITYDEFKHCWDTFVDHQGCPFQPWDGLDEHSQALSGRL
RAELQNQGNSGGSSGGSSGSETPGTSESATPESSGGSSGGSDKKYSIGLAIGT
NSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNS
DVDKLFIQLVQTYNQLFEENPINASGVDAKNLSARLSKSRRLENLIAQLPGEKK
NGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLR
KQRTFDNGSIPHQIHLGELHAILRRQEDFYPFLKDNREKEEKILTFRIPYYVGPLA
RGNSRFAWMTRKSEETITPWNFEEVVDKGASAQSFIERMTNFDKNLPNEKVLP
KHSLLYEYFTVYNELTKVKYVTEGMRKPAFLSGEQKKANDLLSKTNRKVTVKQ
LKEDYFKKIECFDSVEISGVEDRFNASLGTYHDLLKIIKDKDFLDNEENEDELEDIV
LTLTLFEDREMEEERLKTYAHLFDDKVMKQLKRRRYTGWGRLSRKLINGIRDKQ
SGKTILDFLKSDGFANRNFMLIHDDSLTFKEDEQKAQVSGQGDSLHEHIANLA
GSPAIKKGILQTVKVVDELVKVNAGRHKPENIVIEMARENVTQKGQKNSRERMK
RIEEGIKELGSQLKEHPVENTQLONEKLYLYYLQNGRDMWDQELDINRLSDYD
VDHIVPQSFLKDDSEDNKVLTRSDKNRGKSDNVPSEEVVKKMKYWRQLLNAK
LITQRKFDNLTKAERGGLSELDKAGFEKRQLVETRQITKHVAQILDSRMNTKYDE
NDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEHEQISE
FSKRVILADANLDKVLSAYNKHRDKPIREQAENIIHLFTLTNLGAPAAFKYFDTTID
RKRYTSTKEVLDATLIHQSITGLYETRIDLSQLGGDSGGSKRTADGSEFEPKKK
RKVSGGSGGSGGSTNLSDIEKETGKQLVIQESILMLPEEVEEVIGNKPESDILVH
TAYDESTDENVMLLTSDAPEYKPWALVIQDSNGENKIKVILSGGSGGSGGSTNL
SDIIEKETGKQLVIQESILMLPEEVEEVIGNKPESDILVHTAYDESTDENVMLLTS
DAPEYKPWALVIQDSNGENKIKMLGSGATNFSLIKQAGDVEENPGPMVSKGEE
LFTGVVPILVELDGEWNGHKFSVSGEGEGDATYGKLTLKFICTTGKLPVPWPTL
VTTLTYGVQCFSRYPDHMKQHDFFKSAMPEGYVQERTIFFKDDGNYKTRAEVK
FEGDTLVNRIELKGIDFKEDGNILGHKLEYNYNSHNVYIMADKQKNGIKVNFKIR
HNIEDGSVQLADHYQQNTPIGDGPVLIPDNHYLSTQSALSKOPNEKRDHMVLL
EFVTAAGITIGMDELYKSGGSPKKKRKV
pCMV-bpNLS-TadA*(ABE8e)-48AA-hA3A-nCas9-bpNLS-P2A-EGFP, SEQ ID
NO: 166
MKRTADGSEFESPKKKRKVSEVEFSHEYWMRHALTLAKRARDEREVPVGAVL
VLNNRVIGEGWNRAIGLHDPTAHAEIMALRQGGLVMQNYRLIDATLYVTFEPCV
MCAGAMIHSRIGRVVFGVRNSKRGAAGSLMNVLNYPGMNHRVEITEGILADEC
AALLCDFYRMPRQVFNAQKKAQSSINSGSETPGTSESATPESSGGSSGGSSG
SETPGTSESATPESSGGSSGGSEASPASGPRHLMDPHIFTSNFNNGIGRHKTY
LCYEVERLDNGTSVKMDCHRGFLHNQAKNLICGFYGRHAELRFLDLVPSLQLD
PAQIYRVTWFISWSPCFSWGCAGEVRAFLQENTHVRLRIFAARIYDYDPLYKEA
LQMLRDAGAQVSIMTYDEFKHCWDTFVDHQGCPFQPWDGLDEHSQALSGRL
RAILQNQGNSGGSSGGSSGSETPGTSESATPESSGGSSGGSDKKYSIGLAIGT
NSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPQNS
DVDKLFIQLVQTYNQLFEENPINASGVDAKNLSARLSKSRRLENLIAQLPGEKK
NGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLP
KHSLLYEYFTVYNELTKVKYVTEGMRKPAFLSGEQKKANDLLSKTNRKVTVKQ
LKEDYFKKIECFDSVEISGVEDRFNASLGTYHDLLKIIKDKDFLDNEENEDILEDIV
LTLTLFEDREMEEERLKTYAHLFDDKVMKQLKRRRYTGWGRLSRKLINGIRDKQ
SGKTILDFLKSDGFANRNFMLIHDDSLTFKEDEQKAQVSGQGDSLHEHIANLA
GSPAIKKGILQTVKVVDELVKVNAGRHKPENIVIEMARENQTTQKGQKNSRERMK
RIEEGIKELGSQLKEHPVENTQLQNEKLYLYYLQNGRDMYVDQELDINRLSDYD
VDHIVPQSFLKDDSEDNKVLTRSDKNRGKSDNVPSEEVVKMIKNYWRQLLNAK
LITQRKFDNLTKAERGGLSELDKAGFEKRQLVETRQITKHVAQILDSRMNTKYDE
NDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEHEQISE
FSKRVILADANLDKVLSAYNKHRDKPIREQAENIIHLFTLTNLGAPAAFKYFDTTID
RKRYTSTKEVLDATLIHQSITGLYETRIDLSQLGGDSGGSKRTADGSEFEPKKK
RKVGSGATNFSLIKQAGDVEENPGPMVSKGEELFTGVVPILVELDGDVNGHKF
SVSGEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIG
DGPVLLPDNHYLSTQSALSKDPNEKRDHMVLLEFVTAAGITLGVIDELYKSGGS
PKKKRKV
pCMV-bpNLS-hA3A-32AA-TadA*(ABE8e)-nCas9-bpNLS-UGI-UGI-P2A-
EGFP, SEQ ID NO: 167
MKRTADGSEFESPKKKRKVEASPASGPRHLMDPHIFTSNFNNGIGRHKTYLCY
EVERLDNGTSVKMDQHRGFLHNQAKNLLOGFYGRHAELRFLDLVPSLQLDPA
QIYRVPATISWSPCFSWGCAGEVRAFLQENTHVRLRIFAARIYDYDPLYKEALQ
MLRDAGAQVSIMTYDEFKHOWDTFVDHQGCPFQPWDGLDEHSQALSGRLRAI
LQNQGNSGGSSGGSSGSETPGTSESATPESSGGSSGGSSEVEFSHEYWMRH
ALTLAKRARDEREVPVGAVLVLNNRVIGEGWNRAIGLHOPTAHAEIMALRQGGL
VMQNYRLIDATLYVTFEPCVMCAGAMIHSRIGRVVFGVIRNSKRGAAGSLMNVL
NYPGMNHRVEITEGILADECAALLCDFYRMPRQVFNAQKKAOSSINSGGSSGG
SSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEI
FSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNF
KSNFDLAEDAKLQLSKQTYDDDLDNLLAQIGDQYADLFLAAKNLSDAILLSDILRV
NTEITKAPLSASIMIKRYDEHHQDLTLIKALVRQQLPEKYKEIFFDQSKNGYAGYI
DGGASQEEFYKFIKPILEKIMDGTEELLVKLAREDLLRKORTFDNGSPHQIHLGE
LHAILRRQEDFYPFLKDNREKIEKILTFRIPYYVGPLARGNSRFAVVIMTRKSEETIT
PWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELV
KVMGRHKPENEVIEMARENQTTQKGQKNSRERMKRIEEGIKELGSQILKEHPVE
NTQLQNEKLYLYYLQNGRDNAYVDQELDENRLSDYDVDHIVPQSFLKDDSIDNKV
LTRSDKNRGKSDNVPSEEVVKKIMKNYWROLLNAKLITQRKFDNLTKAERGGLS
ELDKAGFIKRQLVETRQITKHVAQILDSRMNTKYDENDKLIREVKVITLKSKLVSD
FRKDFQFYKVREENNYHHAHDAYLNAVVGTALIKKYPKLESEFVYGDYKVYDVR
KMIAKSEQEIGKATAKYFFYSNIMNFFKTEITLANGEIRKRPLIETNGETGEIVWD
KGRDFATVRKVLSMPQVNEVKKTEVQTGGFSKESILPKRNSDKLIARKKDWDPK
KYGGFDSPTVAYSVLVVAKVEKGKSKKLKSVKELLGITIMERSSFEKNPIDFLEA
KGYKEVKKDLIIKLPKYSLFELENGRKRNALASAGELQKGNELALPSKYVNFLYLA
SHYEKLKGSPEDNEQKQLFVEQHKHYLDEHEQISEFSKRVILADANLDKVLSAY
NKHRDKPIREQAENIIHLFTLTNLGAPAAFKYFDTTIDRKRYTSTKEVLDATLIHQ
SITGLYETRIDLSQLGGDSGGSKRTADGSEFEPKKKRKVSGGSGGSGGSTNLS
DIIEKETGKQLVIQESILMLPEEVEEVIGNKPESDILVHTAYDESTDENVMLITSD
APEYKPWALVIQDSNGENKIKVILSGGSGGSGGSTNLSDIIEKETGKQLVIQESIL
MLPEEVEEVIGNKPESDILVHTAYDESTDENVIVILLTSDAPEYKPWALVIQDSNG
ENKIKMLGSGATNFSLLKQAGDVEENPGPMVSKGEELFTGVVPILVELDGDVN
GHKFSVSGEGEGDATYGKLTLKFICTTGKLPVPWPTLVTTLTYGVQCFSRYPD
HMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQ
NTPIGDGPVLLPDNHYLSTQSALSDPNEKRDHMVLLEFVTAAGITLGMDELYK
SGGSPKKKRKV
pCMV-bpNLS-hA3A-32AA-TadA*(ABE8e)-nCas9-bpNLS-P2A-EGFP, SEQ ID
NO: 168
MKRTADGSEFESPKKKRKVEASPASGPRHLMDPHIFTSNFNNGIGRHKTYLCY
EVERLDNGTSVKMDQHRGFLHNQAKNLLOGFYGRHAELRFLDLVPSLQLDPA
QIYRVPATISWSPCFSWGCAGEVRAFLOENTHVRLRIFAARIYDYDPLYKEALQ
MLRDAGAQVSIMTYDEFKHCWDTFVDHQGCPFQPWDGLDEHSQALSGRLRAI
LQNQGNSGGSSGGSSGSETPGTSESATPESSGGSSGGSSEVEFSHEYWMRH
ALTLAKRARDEREVPVGAVLVLNNRVIGEGWNRAIGLHDPTAHAEIMALRQGGL
VMQNYRLIDATLYVTFEPCVMCAGAMIHSRIGRVVFGVRNSKRGAAGSLMNVL
NYPGMNHRVEITEGILADECAALLCDFYRMPRQVFNAQKKAOSSINSGGSSGG
SSGSETPGTSESATPESSGGSSGGSDKKYSIGLAIGTNSVGWAVITDEYKVPSK
KFKVLGNTDRHSIKKNLIGALLFDSGETAEATRLKRTARRRYTRRKNRICYLQEI
FSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNOLF
EENPINASGVDAKAILSARLSKSRRLENLIAOLPGEKKNGLFGNLIALSLGLTPNF
KSNFDLAEDAKLQLSKQTYDDDLDNLLAQIGDQYADLFLAAKNLSDAILLSDILRV
NTEITKAPLSASIMIKRYDEHHQDLTLIKALVRQQLPEKYKEIFFDQSKNGYAGYI
DGGASQEEFYKFIKPILEKIMDGTEELLVKLAREDLLRKORTFDNGSPHQIHLGE
LHAILRRQEDFYPFLKDNREKIEKILTFRIPYYVGPLARGNSRFAVVIMTRKSEETIT
PWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYA
HLFDDKVNAKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELV
KVMGRHKPENEVIEMARENQTTQKGOKNSRERNAKRIEEGIKELGSQILKEHPVE
NTQLQNEKLYLYYLQNGRDMWDQELIDENRLSDYDVDHIVPQSFLKDDSIDNKV
LTRSDKNRGKSDNVPSEEVVKKMKNYWROLLNAKLITQRKFDNLTKAERGGLS
ELDKAGFIKRQLVETRQITKHVAQILDSRMNTKYDENDKVREVKVITLKSKLVSD
FRKDFQFYKVREENNYHHAHDAYLNAVVGTALIKKYPKLESEFWGDYKVYDVR
KMIAKSEQEIGKATAKYFFYSNAINFFKTEITLANGEIRKRPLIETNGETGERAND
KGRDFATVRKVLSMPQVNEVKKTEVQTGGFSKESILPKRNSDKLIARKKDWDPK
KYGGFDSPTVAYSVLVVAKVEKGKSKKLKSVKELLGITIMERSSFEKNPIDFLEA
KGYKEVKKDLIIKLPKYSLFELENGRKRNALASAGELQKGNELALPSKYVNFLYLA
SHYEKLKGSPEDNEQKQLFVEQHKHYLDEHEQISEFSKRVILADANLDKVLSAY
NKHRDKPIREQAENIIHLFTLTNLGAPAAFKYFDTTIDRKRYTSTKEVLDATLIHQ
SITGLYETRIDLSQLGGIDSGGSKRTADGSEFEPKKKRKVGSGATNFSLLKCAG
DVEENPGPMVSKGEELFTGVVPILVELDGDVNGHKFSVSGEGEGDATYGKLTL
KFICTTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSA
LSKDPNEKRDHMVLLEFVTAAGITLGNADELYKSGGSPKKKRKV
miniABEmax-V82G, SEQ ID NO: 223:
MKRTADGSEFESPKKKRKVSEVEFSHEYWMRHALTLAKRARDEREVPVGAVLV
LNNRVIGEGWNRAIGLHDPTAHAEIMALRQGGLVMQNYRLIDATLYGTFEPCV
MCAGAMIHSRIGRWFGVRNAKTGAAGSLMDVIHYPGMNHRVEITEGILADEC
AALLCYFFRMPRQVFNAQKKAQSSTDSGGSSGGSSGSETPGTSESATPESSGGSS
GGSDKKYSIGLAIGTNSVGWAVITDEYKYTSKKFKVLGNTDRHSIKKNLIGALLF
DSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIK
FRGHELIEGDLNPDNSDVDKLFIQLVQTYNQLFEENPINASGVDAKAFLSARLSKS
RRLENLIAQLPGEKKNGLFGNLIALSLGLTPNFKSNFDLAEDAKLQLSKDTYDDD
LDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVD
LLFKTNRKVIVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYFFYSN
IMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKR
TADGSEFEPKKKRKVGSGATNFSLLKQAGDVEENPGPMVSKGEELFTGWPBLV
ELDGDVNGHKFSVSGEGEGDATYGKLTLTLKFICTTGKLPVPWPTLVTTLTYGVQC
FSRYPDHMKQHDFFKSAMPEGYVQERTIFFKDDGNYKIRAEVKFEGDTLVNRIE
LKGIDFKEDGNILGHKLEYNYNSHNVYIMADKQKNGIKVNFKIRHNIEDGSVQL
ADHYQQNTPIGDGPVLLPDNHYLSTQSALSKDPNEKRDHMVLLEFVTAAGITLG
MDELYKSGGSPKKKRKV
miniABEmax-K20A/R21A, SEQ TO NO: 224:
MKRTADGSEFESPKKKRKVSEVEFSHEYWMRHALTLAAAARDEREVPVGAVLV
LNNRVIGEGWNRAIGLHDPTAHAEIMALRQGGLVMQNYRLIDATLYVTFEPCV
MCAGAMIHSRIGRVVFGVRNAKTGAAGSLMDVLHYPGMNHRVEITEGILADEC
AALLCYFFRMPRQVFNAQKKAQSSTDSGGSSGGSSGSETPGTSESATPESSGGSS
GGSDKKYSIGLAIGTNSVGWAVITDEYKVTSKKFKVLGNTDRHSIKKNLIGALLF
DSGETAEATRLKRTARRRYTRRKNRTCYLQEFFSNEMAKVDDSFFHRLEESFLVE
EDKKHERHPIFGNIVDEVAYHEKYPTIYHLRKKLVDSTDKADLRLIYLALAHMIK
FRGHFLIEGDLNPDNSDVDKLFIQLVQTYNQLFFENPINASGVDAKAILSARLSKS
RRLENLIAQLPGEKKNGLFGNLIALSLGLTPNFKSNFDLAEDAKLQLSKDTYDDD
LDNLLAQIGDQYADLFLAAKNLSDAILLSDTLRVNTEITKAPLSASMIKRYDEHHQ
DLTLLKALVRQQLPEKYKEIFFDQSKNGYAGYIDGGASQEEFYKFIKPILEKMDG
TEELLVKLNREDLLRKQRTFDNGSIPHQIHLGELHAHJRRQEDFYPFLKDNREKIE
KILTFRIPYYVGPLARGNSRFAWMTRKSEETITPWNFEEVVDKGASAQSFIERMT
KFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIOKAQVSGQ
GDSLHEHIANLAGSPAIKKGILQTVKVVDELAKVMGRHKPENIVIEMARENQTTQ
KGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDA
YLNAWGTALIKKYPKLESEFVYGDYKVYDVRKMIAKSEQEIGKATAKYFFYSN
LYLNFFKTE1TLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKR
TADGSEFEPKKKRKVGSGATNFSLLKQAGDVEENPGPMVSKGEELFTGVVPILV
ELDGDVNGHKFSVSGEGEGDATYGKLTLKFICTTGKLPVPWPTLVTTLTYGVQC
FSRYPDMMKQHDFFKSAMPEGYVQERTIFFKDDGNYKTRAEVKFEGDTLVNRIE
LKGIDFKEDGNILGHKLEYNYNSHNVYIMADKQKNGIKVNFKJRHN1EDGSVQL
ADHYQQNTPIGDGPVLLPDNHYLSTQSALSKDPNERDHMVLLEFVTAAGITLG
MDELYKSGGSPKKKRKV
miniABEmax-V106W, SEQ ID NO: 225:
MKRTADGSEFESPKKKRKVSEVEFSHEYWMRHALTLAKRARDEREVPVGAVLV
LNNRVIGEGWNRAIGLHDPTAHAEIMALRQGGLVMQNYRLIDATLYVTFEPCV
MCAGAMIHSRIGRWTGWRNAKTGAAGSLMDVLHYPGMNHRVEITEGILADEC
AALLCYFFRMPRQVFNAQKKAQSSTDSGGSSGGSSGSETPGTSESATPESSGGSS
GGSDKKYSIGLAIGTNSVGWAVITDEYKWSKKFKVLGNTDRHSIKKNLIGALLF
DSGETAEATRLKRTARRRYTRRKNRICYLQEIFSNEMAKVDDSFFHRLEESFLVE
EDKKHERHPIFGNIYDEVAYHEKYPTIYHLRKKLVDSTDKADLRLIYLALAHMIK
FRGHFLIEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKDTYDDD
LDNLLAQIGDQYADLFLAAKNLSDAILLSDILRVTMTEITKAPLSASMIKRYDEHHQ
DLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMT
NFDKNLPNEKVLPKHSLLYEYFWYNELTKVKYVTEGMRKPAFLSGEQKKAIVD
LLFKTNRKVTVKQLKEDYFKKIECFDSVEISGVEDRFNASLGTYHDLLKIIKDKDF
LDNEENEDDLEDIVLTLTLFEDREMIEERLKTYAHLFDDKVMKQLKRRRYTGWG
RLSRKINGIRDKQSGKTILDFLKSDGFANRNFMQLIHDDSLTFKEDIQKAQVSGQ
GDSLHEHTANLAGSPAIKKGILQWKVVDELVKVMGRITKPENIVIEMARENQTTQ
KGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEWKKM
KNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDA
YLNAVVGTALIKKYPKLESEFVYGDYKVYDVRKMIAKSEOEIGKATAKYFFYSN
IMNFFKTEITLANGEIRKRPLIETNGETGEJVWDKGRDFATVRKVLSMPQVNrVKK
TEVQTGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKYSLFEUEN
GRKRMLASAGELQKGNELALPSKYVNFLYLASHYEKLKGSPEDNEQKQLFVEQ
FrKHYLDEFIEQISEFSKRVILADANLDKVLSAYNKHRDKPIREQAENTIHLFTLTNL
GAPAAFKYFDTTIDRKRYTSTKEVLDATLIHQSITGLYETRIDLSQLGGDSGGSKR
TADGSEFEPKKKRKVGSGATNFSLLKQAGDVEENPGPMVSKGEELFTGVVPILV
ELDGDVNGHKFSVSGEGEGDATYGKLTLKFICTTGKLPVPWPTLVTTLTYGVQC
FSRYPDIIMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQL
ADIIYQQNTPIGDGPVLLPDNHYLSTQSALSKDPNEKRDIIMVLLEFVTAAG1TLG
MDELYKSGGSPKKKRKV
TadA* from ABE7.10 and ABEmax (SEQ ID: 226):
SEVEFSHEYWNARHALTLAKRARDEREVPVGAVLVLNNRVIGEGWNRAIGLHDP
TAHAEIMALRQGGLVMQNYRLIDATLYVTFEPCVMCAGAMIHSREGRVVFGVRN
AKTGAAGSLMDVLHYPGMNHRVEITEGILADECAALLCYFFRMPRQVFNAQKK
AQSSTD.
TadA(WT)-TadA* heterodimer from ABE7.10 and ABEmax (SEQ ID: 227):
MSEVEFSHEYWMRHALTLAKRAWDEREVPVGAVLVHNNRVIGEGWNRPIGRH
DPTAHAEIMALRQGGLVMQNYRLIDATLYVTLEPCVMCAGAMIHSRIGRVVFGA
RDAKTGAAGSLMDVLHHPGMNHRVEITEGILADECAALLSDFFRMRRQEIKAQK
KAQSSTDSGGSSGGSSGSETPGTSESATPESSGGSSGGSSEVEFSHEYWMR
HALTLAKRARDEREVPVGAVLVLNNRVIGEGWNRAIGLHDPTAHAEIMALRQGG
LVMQNYRLIDATLYVTFEPCVMCAGAMIHSRIGRVVFGVRNAKTGAAGSLMDVL
HYPGMNHRVEITEGILADECAALLCYFFRMPRQVFNAQKKAQSSTD
ABEmax, SEQ ID NO: 228
MKRTADGSEFESPKKKRKVSEVEFSHEYWMRHALTLAKRAWDEREVPVGAVL
VHNNRVIGEGWNRPIGRHDPTAHAEIMALRQGGLVMQNYRLIDATLYVTLEPCV
MCAGAMIHSRIGRVVFGARDAKTGAAGSLMDVLHHPGMNHRVEITEGILADEC
AALLSDFFRMRRQEIKAQKKAQSSTDSGGSSGGSSGSETPGTSESATPESSG
GSSGGSSEVEFSHEYWMRHALTLAKRARDEREVPVGAVLVLNNRVIGEGWNR
AIGLHDPTAHAEIMALROGGLVMQNYRLIDATLYVTFEPCVMCAGAMIHSRIGR
VVFGVRNAKTGAAGSLMDVLHYPGMNHRVEITEGILADECAALLCYFFRMPRQ
VFNAQKKAQSSTDSGGSSGGSSGSETPGTSESATPESSGGSSGGSDKKYSIG
LAIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQL
PGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLIKALVR
QQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNE
KVLPKHSLLYEYFTVYNELTKVKYVTEGMRKPAFLSGEQKKNVDLLFKTNRKVT
VKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRE
RMKRIEEGIKELGSQILKEHPVENTQLQNEKLYLYYLONGRDMYVDQELDINRL
SDYDVDHIVPQSFLKDDSIDNKVLTRSDKNRGKSDNVPSEEVVKKMKNYWRQL
LNAKLITQRKFDNLTKAERGGLSELDKAGFIKROLVETRQITKHVAQILDSRMNT
KYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKY
FDTTIDRKRYTSTKEVLDATLIHQSITGLYETRIDLSQLGGDSGGSKRTADGSEF
EPKKKRKV

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A bifunctional adenine and cytosine base editor (BACE) comprising:
(i) an adenosine deaminase domain that decreases RNA editing activity while preserving DNA editing activity;
(ii) a cytidine deaminase domain;
(iii) one or multiple uracil-n-clycosalyse inhibitors (UGIs); and
(iv) a programmable DNA binding domain; and
(v) optionally further comprising one or more nuclear localization sequences.

2. The BACE of claim 1, wherein the adenosine deaminase domain comprises a wild type (SEQ ID NO: 98) and/or engineered adenosine deaminase TadA monomer or dimer.

3. The BACE of claim 2, wherein the engineered adenosine deaminase TadA monomer or dimer comprises a homodimeric or heterodimeric TadA domain from ABEmax (SEQ ID NO:226), ABE7.10 (SEQ ID NO:227), or ABE8e (SEQ ID NO: 145); monomer or dimer TadA from ABE 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10 (SEQ ID NO: 139), ABEmax (SEQ ID NO: 228), ABE8.8 (SEQ ID NO: 148), ABE8.13 (SEQ ID NO: 149), ABE8.17 (SEQ ID NO: 150), ABE8.20 (SEQ ID NO: 151), ABE8e (SEQ ID NO: 145), or K20A/R21A, V82G or V106W variants thereof; E. coli TadA monomer, or homo- or heterodimers thereof fused to the N or C terminus, optionally comprising one or more mutations in either or both monomers, optionally TadA from miniABEmax-V82G (SEQ ID 223), miniABEmax-K20A/R21A (SEQ ID 224), miniABEmax-V106W (SEQ ID 225), or another variant listed in Tables C, N, and O).

4. The BACE of claim 1, wherein the adenosine deaminase domain comprises one or more mutations corresponding to E. coli TadA mutations in one or more TadA monomers shown in Table N, or an homologue or orthologue thereof, optionally a TadA protein in Table C.

5. The BACE of claim 1, wherein the cytidine deaminase domain is from Table A or B, preferably pmCDA1, rat APOBEC1, human APOBEC3A, or human AID, or a variation thereof with reduced RNA off-target editing

6. The BACE of claim 5, wherein the cytidine deaminase domain is a rat APOBEC1, or an ortho- or paralogue thereof as listed in Tables A or B, optionally comprising one or more mutations that decrease RNA editing activity while preserving DNA editing activity, preferably wherein the mutations are at amino acid positions that correspond to residues P29, R33, K34, W90Y, R126E, R132E, E181, and/or L182 of rat apolipoprotein B mRNA editing enzyme, catalytic polypeptide-like 1 (rAPOBEC1, SEQ ID NO:67).

7. The BACE of claim 6, wherein the cytidine deaminase domain comprises one or more mutations corresponding to P29F, P29T, R33A, K34A, R33A+K34A (double mutant), W90Y, R126E, R132E, W90Y+R126E (double mutant), R126E+R132E (double mutant), W90Y+R132E (double mutant), W90Y+R126E+R132E (triple mutant), E181Q and/or L182A of SEQ ID NO:67 (rAPOBEC1, Rattus norvegicus APOBEC1).

8. The BACE of claim 7, wherein the cytidine deaminase domain further comprises one or more mutations at residues corresponding to E24, V25; R118, Y120, H121, R126; W224-K229; P168-I186; L173+L180; R15, R16, R17, to K15-17 & A15-17; Deletion E181-L210; P190+P191; Deletion L210-K229 (C-terminal); and/or Deletion S2-L14 (N-terminal) of SEQ ID NO:67, Table O.

9. The BACE of claim 1, wherein the one or more nuclear localization sequences comprise a bipartite NLS comprising the sequence KRTADGSEFEPKKKRKV (SEQ ID NO:229); an SV40 large T antigen NLS (PKKKRRV (SEQ ID NO:221)); or a nucleoplasmin NLS (KRPAATKKAGQAKKKK (SEQ ID NO:222)).

10. The BACE of claim 1, comprising a linker between the adenosine deaminase domain and/or between the adenosine deaminase domain or single-chain dimers and the programmable DNA binding domain.

11. (canceled)

12. The BACE of claim 1, comprising:
(i) an N-terminal adenosine deaminase domain, preferably mutant TadA* monomer or dimer, and a C-terminal cytidine deaminase domain, preferably pmCDA1 or rAPOBEC1 or hA3A or AID, or
(ii) an N-terminal cytidine deaminase domain, preferably pmCDA1 or rAPOBEC1 or hA3A or AID, and a C-terminal adenosine deaminase domain, preferably mutant TadA* monomer or dimer.

13. The BACE of claim 1, comprising a heterodimeric combined N-terminal adenosine and cytidine deaminase fusion, preferably pmCDA1 or rAPOBEC1 or hA3A or AID fused to one or more TadA monomers or dimers with an intervening linker, or a heterodimeric combined C-terminal adenosine and cytidine deaminase fusion, preferably pmCDA1 or rAPOBEC1 or hA3A or AID fused to one or more TadA monomers or dimers with an intervening linker.

14. The BACE of claim 1, wherein the programmable DNA binding domain is selected from the group consisting of engineered C2H2 zinc-fingers, transcription activator effector-like effectors (TALEs), and Clustered Regularly Interspaced Short Palindromic Repeats (CRISPR) Cas RNA-guided nucleases (RGNs) and variants thereof.

15. The BACE of claim 1, wherein the CRISPR RGN is an ssDNA nickase or is catalytically inactive.

16. The BACE of claim 1, wherein the CRISPR RGN is a Cas9, CasX, or Cas12a that has ssDNA nickase activity or is catalytically inactive.

17. The BACE of claim 16, wherein the programmable DNA binding domain comprises Cas9-D10A, CasX with Asp672, Glu769 and Asp935 mutated to alanine separately or together to obtain dDpbCasX, dAsCpf1 (D908A), or dLbCpf1 (D832A).

18. The BACE of claim 1, comprising a sequence that is at least 80% identical to one of SEQ ID NOs:140-144.

19. Abase editing system comprising:
(i) The BACE of claim 1, wherein the programmable DNA binding domain is a CRISPR Cas RGN or a variant thereof; and
(ii) at least one guide RNA compatible with the base editor that directs the base editor to a target sequence.

20. An isolated nucleic acid encoding the BACE of claim 1.

21. A vector comprising the isolated nucleic acid of claim 20.

22. An isolated host cell, preferably a mammalian host cell, comprising the nucleic acid of claim 20.

23. The isolated host cell of claim 22, which expresses the BACE of claim 1.

24. A method of deaminating a selected adenine and/or cytosine in a target sequence in a nucleic acid, the method comprising contacting the nucleic acid with the BACE of claim 1.

25. The method of claim 24, wherein the programmable DNA binding domain is a CRISPR Cas RGN or a variant thereof, and the method further comprises contacting the nucleic acid with at least one guide RNA compatible with the base editor that directs the base editor to the target sequence.

26. A composition comprising a purified the base editing system f claim 19.

27. (canceled)

28. The composition of claim 26, comprising one or more ribonucleoprotein (RNP) complexes.

29. A method of inducing an amino acid change in a polypeptide, the method comprising contacting a nucleotide sequence that encodes the polypeptide to be modified with the BACE of any claim 1, optionally wherein the amino acid change comprises one of the amino acid changes listed in Table D, optionally wherein the amino acid change is one that can or cannot be targeted by CBE and/or ABE.

30. The method of claim 29, wherein the amino acid change corrects a disease-related mutation shown in Tables E, I-K, or introduces a multi-nucleotide variant (MNV) as shown in Tables F-H.

31. A method of generating two or more sets of nucleic acids, each set comprising a plurality of sequences, wherein each set comprises one or more nucleic acids having the same sequence, and wherein each set differs from each of the other sets by at least one nucleotide, the method comprising:
(i) providing a first nucleic acid comprising a first sequence;
(ii) contacting the first set of nucleic acids with the BACE of claim 1, wherein the programmable DNA binding domain is a CRISPR Cas RGN or a variant thereof; and a least one guide RNA compatible with the base editor that directs the base editor to alter a first nucleotide in the first sequence;
(iii) isolating a second nucleic acid comprising a sequence comprising at least one modification in the nucleotide sequence as compared to the first nucleic acid, and optionally amplifying the second nucleic acid, to provide a second set of nucleic acids; and
(iv) optionally repeating steps (i)-(iii) until a desired number of sets is obtained.

32. The method of claim 31, comprising repeating steps (i)-(iii) until a plurality of sets are obtained comprising a mutation at each position in a selection region of the sequence of the nucleic acid.
